(19) United States
(12) Patent Application Publication
(10) Pub. No.: US 2020/0376034 A1
(43) Pub. Date: Dec. 3, 2020

(54) ANTIBODY VARIABLE DOMAINS TARGETING CD33, AND USE THEREOF

(71) Applicant: Dragonfly Therapeutics, Inc., Waltham, MA (US)

(72) Inventors: Gregory P. Chang, Medford, MA (US); Ann F. Cheung, Lincoln, MA (US); Asya Grinberg, Lexington, MA (US); Dhruv Kam Sethi, Belmont, MA (US); William Haney, Wayland, MA (US); Bianka Prinz, Lebanon, NH (US)

(21) Appl. No.: 16/971,098

(22) PCT Filed: Feb. 20, 2019
(86) PCT No.: PCT/US19/18748

§ 371 Date: Aug. 19, 2020

Related U.S. Application Data
(60) Provisional application No. 62/632,756, filed on Feb. 20, 2018.

(51) Int. Cl. A61K 35/17; A61P 35/02; C07K 16/28

(52) U.S. Cl. CPC A61K 35/17; A61P 35/02; A61K 47/6817; C07K 16/2851; C07K 16/2803

(57) ABSTRACT

Disclosed are proteins with antibody heavy chain and light chain variable domains that can be paired to form an antigen binding site targeting CD33 (Siglec-3) on a cell, pharmaceutical compositions comprising such proteins, and therapeutic methods using such proteins and pharmaceutical compositions, including for the treatment of cancer.

### CROSS-REFERENCE TO RELATED APPLICATIONS

This application claims the benefit of and priority to U.S. Provisional Patent Application No. 62/632,756, filed Feb. 20, 2018, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Feb. 19, 2019, is named DFY_050WO_SL25.txt and is 571,095 bytes in size.

### FIELD OF THE INVENTION

The invention provides proteins with antibody heavy chain and light chain variable domains that can be paired to form an antigen binding site targeting CD33 (Siglec-3) on a cell, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers in adults include prostate cancer, breast cancer, and lung cancer. Hematological malignancies, though less frequent than solid cancers, have low survival rates. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. T cells are major effectors of the adaptive immune system that attack foreign cells as well as host cells that present mutant or mis-expressed peptides. Cells targeted by T cells may be virally-infected, such that they express foreign proteins, or malignant, where they might express mutant proteins. T cells recognize target cells via their T cell receptor (TCR) engaging intracellular peptides presented by major histocompatibility complex proteins on target cells. Individual T cells typically recognize target cells bearing specific MHC-peptide complexes, but novel agents have been developed that usurp and amplify this natural process for therapeutic benefit. Bi-specific T cell engagers link antigen binding site(s) of tumor-associated antigens to antigen binding site(s) of components of the TCR complex to redirect T cell activity towards desired target cells independent of native peptide-MHC recognition. For example, Blincyto is an FDA-approved T cell engager that targets CD19 on malignant B cells.

T cells can also be engineered to express chimeric antigen receptors (CAR) that endow it with target recognition capabilities of its CAR. CARs contain antigen binding site(s) to tumor associated antigens linked to T cell activation domains. These CAR-T cells can also be employed to target malignant cells, and some have been FDA-approved for use against B cell malignancies.

Antibodies that bind to certain tumor-associated antigens and to certain immune cells have been described in the literature. See, e.g., WO 2016/134371 and WO 2015/095412. Antibody-drug conjugates or immunocytokines using antigen binding sites targeting tumor associated antigens to deliver toxic agents or immune-modulatory cytokines to specific target cells.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-gamma and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

CD33 is a member of the sialic acid-binding immunoglobulin-like lectins. As a transmembrane receptor mainly expressed on cells of myeloid lineage, CD33 modulates inflammatory and immune responses through a dampening effect on tyrosine kinase-driven signaling pathways. For example, CD33 was shown to constitutively suppress the production of pro-inflammatory cytokines such as IL-1β, TNF-α, and IL-8 by human monocytes.

CD33 is associated with hematopoietic cancers. It is broadly expressed in blasts of nearly all acute myeloid leukemia (AML). Furthermore, hematopoietic cancer stem and/or progenitor cells are found to be CD33⁺, implying that CD33-directed therapy could potentially eradicate malignant stem and/or progenitor cells in such cases while sparing normal hematopoietic stem cells. In addition to its expression in AML, CD33 is found on other myeloid neoplasms (e.g., myelodysplastic syndromes and myeloproliferative neoplasms) and on subsets of B-cell and T-cell acute lymphoblastic leukemias (ALL)/lymphoblastic lymphomas. This expression pattern has led to the use of CD33-directed therapeutics in patients with malignancies including AML, myelodysplastic syndromes, chronic myelomonocytic leukemia, myeloid blast crisis of chronic myeloid leukemia, and ALLs.

### SUMMARY OF THE INVENTION

In one aspect, the invention provides an antigen binding site that binds an epitope on an extracellular domain of human CD33 and/or Cynomolgus/Rhesus (cyno) CD33. In one aspect, the present invention provides an antigen binding site that recognizes and binds one or more conformational epitopes on the extracellular domain of human CD33 and/or Cynomolgus/Rhesus (cyno) CD33. In one aspect, the present invention provides an antigen binding site that recognizes and binds one or more conformational epitopes on the extracellular domain of human CD33 but does not recognize and/or bind one or more conformational epitopes on the extracellular domain of cyno CD33. In one aspect, the present invention provides an antigen binding site that binds to the R69G allele of human CD33. In one aspect, the present invention provides an antigen binding site that binds to wild-type human CD33 (e.g., having the amino acid sequence identified by NCBI Reference Sequence: NP_001763.3) but not the R69G allele of human CD33. In one aspect, the present invention provides an antigen binding site that binds to an epitope on human CD33 that includes R69. In one aspect, the present invention provides an antigen binding site including a heavy chain variable domain that binds to the extracellular domain in human CD33 and/or cyno CD33, irrespective of the glycosylation profile of the targeted CD33.

In certain embodiments, the present invention provides an antigen binding site that binds to the extracellular domain of human CD33 and/or cyno CD33, such that the epitopes are unique compared to the epitopes targeted by one or more known anti-CD33 antibodies in the art. In certain embodiments, the present invention provides an antigen binding site that binds to the extracellular domain of human CD33 and/or cyno CD33, and shows human or cyno CD33 cross-reactivity and high affinity binding to the target CD33.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVQPGGSLRLSCAASGFTFSSYGMSWVRQAPGKGLEWVANIKQDGS EKYYVDSVKGRFTISRDNAKNSLYLQMNSLRAEDTAVYYCAREGGPYYDSSGYFVY YGMDVWGQGTTVTVSS [SEQ ID NO:1]. In some embodiments, the antibody heavy chain variable domain is at least 95% identical to SEQ ID NO:1. In some embodiments, the heavy chain variable domain includes amino acid sequences FTFSSYGMS [SEQ ID NO:21] as the first complementarity-determining region 1 (“CDR1”), NIKQDGSEKYYVDSVKG [SEQ ID NO:22] as the second CDR (“CDR2”), and AREGGPYYDSSGYFVYYGMDV [SEQ ID NO:23] as the third CDR (“CDR3”) of SEQ ID NO:1. In some embodiments, the heavy chain variable domain includes amino acid sequences SYGMS [SEQ ID NO:434] as the first complementarity-determining region 1 (“CDR1”), NIKQDGSEKYYVDSVKG [SEQ ID NO:22] as the second CDR (“CDR2”), and EGGPYYDSSGYFVYYGMDV [SEQ ID NO:435] as the third CDR (“CDR3”) of SEQ ID NO:1. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:1 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYDASSLESGV PSRFSGSGSGTEFTLTISSLQPDDFATYYCQQYESFPTFGGGTKVEIK [SEQ ID NO:2]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:2, which includes amino acid sequences RASQSISSWLA [SEQ ID NO:24] as CDR1, DASSLES [SEQ ID NO:25] as CDR2, and QQYESFPT [SEQ ID NO:26] as CDR3 of SEQ ID NO:2.

In certain embodiments, the antigen-binding site is comprised within an antibody, e.g., a monoclonal antibody, for example, an antibody that comprises a heavy chain variable domain comprising the amino acid sequence of SEQ ID NO:1 and a light chain variable domain comprising the amino acid sequence of SEQ ID NO:2; or an antibody that comprises heavy chain CDRs 1-3 having the sequences of SEQ ID NOs: 21, 22, and 23, respectively, or SEQ ID NOs: 434, 22, and 435, respectively; and light chain CDRs 1-3 having the sequences of SEQ ID NOs: 24, 25, and 26, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANIKQDG SEKYYVDSVKGRFTISRDNAKNSLYLQMNSLRAEDTAVYYCARPLNAGELDVWGQ GTMVTVSS [SEQ ID NO:3]. In some embodiments, the antibody heavy chain variable domain is at least 95% identical to SEQ ID NO:3. In some embodiments, the heavy chain variable domain incorporates amino acid sequences FTFSSYWMS [SEQ ID NO:27] as CDR1, NIKQDGSEKYYVDSVKG [SEQ ID NO:28] as CDR2, and ARPLNAGELDV [SEQ ID NO:29] as CDR3 of SEQ ID NO:3. In some embodiments, the heavy chain variable domain incorporates amino acid sequences SYWMS [SEQ ID NO:181] as CDR1, NIKQDGSEKYYVDSVKG [SEQ ID NO:28] as CDR2, and PLNAGELDV [SEQ ID NO:436] as CDR3 of SEQ ID NO:3. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:3 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 90%, 91%, 92%, 93%, 94%, 95%, 96%, 97%, 98%, 99%, or 100%) identical at least 90% (e.g., 90%, 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQLESYPLTFGGGTKVEIK [SEQ ID NO:4]. In certain embodiments, an antibody heavy chain variable domain at least 90% (e.g., 90%, 91%, 92%, 93%, 94%, 95%, 96%, 97%, 98%, 99%, or 100%) identical at least 90% (e.g., 90%, 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:4, which includes amino acid sequences RASQSISSWLA [SEQ ID NO:30] as CDR1, EASSLES [SEQ ID NO:31] as CDR2, and QQLESYPLT [SEQ ID NO:32] as CDR3 of SEQ ID NO:4.

In certain embodiments, the antigen-binding site is comprised within an antibody, e.g., a monoclonal antibody, for example, an antibody that comprises a heavy chain variable domain comprising the amino acid sequence of SEQ ID NO:3 and a light chain variable domain comprising the amino acid sequence of SEQ ID NO:4; or an antibody that comprises heavy chain CDRs 1-3 having the sequences of SEQ ID NOs: 27, 28, and 29, respectively, or SEQ ID NOs: 181, 28, and 436, respectively; and light chain CDRs 1-3 having the sequences of SEQ ID NOs: 30, 31, and 32, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLLESGGGLVQPGGSLRLSCAASGFTFSKYTMSWVRQAPGKGLEWVSAIVGSGE STYFADSVKGRFTISRDNSKNTLYLQMNSLRAEDTAVYYCAREGGPYYDSSGYFVY YGMDVWGQGTTVTVSS [SEQ ID NO:5]. In some embodiments, the antibody heavy chain variable domain is at least 95% identical to SEQ ID NO:5. In some embodiments, the heavy chain variable domain incorporates amino acid sequences FTFSKYTMS [SEQ ID NO:33] as CDR1, AIVGSGESTYFADSVKG [SEQ ID NO:34] as CDR2, and AREGGPYYDSSGYFVYYGMDV [SEQ ID NO:35] as CDR3 of SEQ ID NO:5. In some embodiments, the heavy chain variable domain incorporates amino acid sequences KYTMS [SEQ ID NO:183] as CDR1, AIVGSGESTYFADSVKG [SEQ ID NO:34] as CDR2, and EGGPYYDSSGYFVYYGMDV [SEQ ID NO:184] as CDR3 of SEQ ID NO:5. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:5 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYKASSLESGV PSRFSGSGSGTEFTLTISSLQPDDFATYYCQQYDDLPTFGGGTKVEIK [SEQ ID NO:6]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:6, which includes amino acid sequences RASQSISSWLA [SEQ ID NO:36] as CDR1, KASSLES [SEQ ID NO:37] or KASSLE [SEQ ID NO:185] as CDR2, and QQYDDLPT [SEQ ID NO:38] as CDR3 of SEQ ID NO:6.

In certain embodiments, the antigen-binding site is comprised within an antibody, e.g., a monoclonal antibody, for example, an antibody that comprises a heavy chain variable domain comprising the amino acid sequence of SEQ ID NO:5 and a light chain variable domain comprising the amino acid sequence of SEQ ID NO:6; or an antibody that comprises heavy chain CDRs 1-3 having the sequences of SEQ ID NOs: 33, 34, and 35, respectively, or SEQ ID NOs: 183, 34, and 184, respectively; and light chain CDRs 1-3 having the sequences of SEQ ID NOs: 36, 37, and 38, respectively, or CDRs 1-3 having the sequences of SEQ ID NOs: 36, 185, and 38, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of QVQLVQ SGAEVKKPGASVKVSCKASGYTF SDYYMHW VRQAPGQGLEWMGMINP S WGSTSYAQKFQGRVTMTRDTSTSTVYMELSSLRSEDTAVYYCAREAADGFVGERYF DLWGRGTLVTVSS [SEQ ID NO:7]. In some embodiments, the antibody heavy chain variable domain is at least 95% identical to SEQ ID NO:7. In some embodiments, the heavy chain variable domain incorporates amino acid sequences YTFSDYYMH [SEQ ID NO:39] as CDR1, MINPSWGSTSYAQKFQG [SEQ ID NO:40] as CDR2, and AREAADGFVGERYFDL [SEQ ID NO:41] as CDR3 of SEQ ID NO:7. In some embodiments, the heavy chain variable domain incorporates amino acid sequences DYYMH [SEQ ID NO:437] as CDR1, MINPSWGSTSYAQKFQG [SEQ ID NO:40] as CDR2, and EAADGFVGERYFDL [SEQ ID NO:438] as CDR3 of SEQ ID NO:7. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 90%, 91%, 92%, 93%, 94%, 95%, 96%, 97%, 98%, 99%, or 100%) identical to the amino acid sequence DIVMTQSPLSLPVTPGEPASISCRSSQSLLYSNGYNYLDWYLQKPGQSPQLLIYLGSN RASGVPDRF SGSGSGTDFTLKISRVEAEDVGVYYCMQDVALPITFGGGTKVEIK [SEQ ID NO:8]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:8, which includes amino acid sequences RSSQSLLYSNGYNYLD [SEQ ID NO:42] as CDR1, LGSNRAS [SEQ ID NO:43] as CDR2, and MQDVALPIT [SEQ ID NO:44] as CDR3 of SEQ ID NO:8.

In certain embodiments, the antigen-binding site is comprised within an antibody, e.g., a monoclonal antibody, for example, an antibody that comprises a heavy chain variable domain comprising the amino acid sequence of SEQ ID NO:7 and a light chain variable domain comprising the amino acid sequence of SEQ ID NO:8; or an antibody that comprises heavy chain CDRs 1-3 having the sequences of SEQ ID NOs: 39, 40, and 41, respectively, or SEQ ID NOs: 437, 40, and 438, respectively; and light chain CDRs 1-3 having the sequences of SEQ ID NOs: 42, 43, and 44, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVQPGGSLRLSCAASGFTFGSYWMSWVRQAPGKGLEWVATIKQDG SEKSYVDSVKGRF TISRDNAKNSLYLQMNSLRAEDTAVYYCARPLNAGELDVWGQ GTMVTVSS [SEQ ID NO:9]. In some embodiments, the antibody heavy chain variable domain is at least 95% identical to SEQ ID NO:9. In some embodiments, the heavy chain variable domain incorporates amino acid sequences FTFGSYWMS [SEQ ID NO:45] as CDR1, TIKQDGSEKSYVDSVKG [SEQ ID NO:46] as CDR2, and ARPLNAGELDV [SEQ ID NO:47] as CDR3 of SEQ ID NO:9. In some embodiments, the heavy chain variable domain incorporates amino acid sequences SYWMS [SEQ ID NO:181] as CDR1, TIKQDGSEKSYVDSVKG [SEQ ID NO:46] as CDR2, and RPLNAGELDV [SEQ ID NO:182] as CDR3 of SEQ ID NO:9. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:9 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:10]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:10, which includes amino acid sequences RASQSISSWLA [SEQ ID NO:48] as CDR1, EASSLES [SEQ ID NO:49] as CDR2, and QQSQSYPPIT [SEQ ID NO:50] as CDR3 of SEQ ID NO:10.

In certain embodiments, the antigen-binding site is comprised within an antibody, e.g., a monoclonal antibody, for example, an antibody that comprises a heavy chain variable domain comprising the amino acid sequence of SEQ ID NO:9 and a light chain variable domain comprising the amino acid sequence of SEQ ID NO:10; or an antibody that comprises heavy chain CDRs 1-3 having the sequences of SEQ ID NOs: 45, 46, and 47, respectively, or SEQ ID NOs: 181, 46, and 182, respectively; and light chain CDRs 1-3 having the sequences of SEQ ID NOs: 48, 49, and 50, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVQPGGSLRLSCAASGFTFPSYWMSWVRQAPGKGLEWVATIKRDGS EKGYVDSVKGRFTISRDNAKNSLYLQMNSLRAEDTAVYYCARPLNAGELDVWGQG TMVTVSS [SEQ ID NO:11]. In some embodiments, the antibody heavy chain variable domain is at least 95% identical to SEQ ID NO:11. In some embodiments, the heavy chain variable domain incorporates amino acid sequences FTFPSYWMS [SEQ ID NO:51] as CDR1, TIKRDGSEKGYVDSVKG [SEQ ID NO:52] as CDR2, and ARPLNAGELDV [SEQ ID NO:53] as CDR3 of SEQ ID NO:11. In some embodiments, the heavy chain variable domain incorporates amino acid sequences SYWMS [SEQ ID NO:181] as CDR1, TIKRDGSEKGYVDSVKG [SEQ ID NO:52] as CDR2, and PLNAGELDV [SEQ ID NO:439] as CDR3 of SEQ ID NO:11. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:11 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:12]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:12, which includes amino acid sequences RASQSISSWLA [SEQ ID NO:54] as CDR1, EASSLES [SEQ ID NO:55] as CDR2, and QQSQSYPPIT [SEQ ID NO:56] as CDR3 of SEQ ID NO:12.

In certain embodiments, the antigen-binding site is comprised within an antibody, e.g., a monoclonal antibody, for example, an antibody that comprises a heavy chain variable domain comprising the amino acid sequence of SEQ ID NO:11 and a light chain variable domain comprising the amino acid sequence of SEQ ID NO:12; or an antibody that comprises heavy chain CDRs 1-3 having the sequences of SEQ ID NOs: 51, 52, and 53, respectively, or SEQ ID NOs: 181, 52, and 439, respectively; and light chain CDRs 1-3 having the sequences of SEQ ID NOs: 54, 55, and 56, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of QVQLVQSGAEVKKPGASVKVSCKASGYTFGTYYMHWVRQAPGQGLEWMGIINPSR GSTVYAQKFQGRVTMTRDTSTSTVYMELS SLRSEDTAVYYCARGAGYDDEDMDV WGKGTTVTVSS [SEQ ID NO:13]. In some embodiments, the antibody heavy chain variable domain is at least 95% identical to SEQ ID NO:13. In some embodiments, the heavy chain variable domain incorporates amino acid sequences YTFGTYYMH [SEQ ID NO:57] as CDR1, IINPSRGSTVYAQKFQG [SEQ ID NO:58] as CDR2, and ARGAGYDDEDMDV [SEQ ID NO:59] as CDR3 of SEQ ID NO:13. In some embodiments, the heavy chain variable domain incorporates amino acid sequences TYYMH [SEQ ID NO:440] as CDR1, IINPSRGSTVYAQKFQG [SEQ ID NO:58] as CDR2, and GAGYDDEDMDV [SEQ ID NO:441] as CDR3 of SEQ ID NO:13. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:13 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSSVSASVGDRVTITCRASQGIDSWLAWYQQKPGKAPKLLIYAASSLQSG VPSRFSGSGSGTDFTLTISSLQPEDFATYYCQQAHSYPLTFGGGTKVEIK [SEQ ID NO:14]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:14, which includes amino acid sequences RASQGIDSWLA [SEQ ID NO:60] as CDR1, AASSLQS [SEQ ID NO:61] as CDR2, and QQAHSYPLT [SEQ ID NO:62] as CDR3 of SEQ ID NO:14.

In certain embodiments, the antigen-binding site is comprised within an antibody, e.g., a monoclonal antibody, for example, an antibody that comprises a heavy chain variable domain comprising the amino acid sequence of SEQ ID NO:13 and a light chain variable domain comprising the amino acid sequence of SEQ ID NO:14; or an antibody that comprises heavy chain CDRs 1-3 having the sequences of SEQ ID NOs: 57, 58, and 59, respectively, or SEQ ID NOs: 440, 58, and 441, respectively; and light chain CDRs 1-3 having the sequences of SEQ ID NOs: 60, 61, and 62, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVKPGGSLRLSCAASGFTFSSYAMSWVRQAPGKGLEWVSSISSSSEG IYYADSVKGRFTISRDNAKNSLYLQMNSLRAEDTAVYYCAREGGPYYDSSGYFVYY GMDVWGQGTTVTVSS [SEQ ID NO:15]. In some embodiments, the antibody heavy chain variable domain is at least 95% identical to SEQ ID NO:15. In some embodiments, the heavy chain variable domain incorporates amino acid sequences FTFSSYAMS [SEQ ID NO:63] as CDR1, SISSSSEGIYYADSVKG [SEQ ID NO:64] as CDR2, and AREGGPYYDSSGYFVYYGMDV [SEQ ID NO:65] as CDR3 of SEQ ID NO:15. In some embodiments, the heavy chain variable domain incorporates amino acid sequences SYAMS [SEQ ID NO:442] as CDR1, SISSSSEGIYYADSVKG [SEQ ID NO:64] as CDR2, and EGGPYYDSSGYFVYYGMDV [SEQ ID NO:443] as CDR3 of SEQ ID NO:15. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:15 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSTLSASVGDRVTITCRASNSISSWLAWYQQKPGKAPKLLIYEASSTKSGV PSRFSGSGSGTEFTLTISSLQPDDFATYYCQQYDDLPTFGGGTKVEIK [SEQ ID NO:16]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:16, which includes amino acid sequences RASNSISSWLA [SEQ ID NO:66] as CDR1, EASSTKS [SEQ ID NO:67] as CDR2, and QQYDDLPT [SEQ ID NO:68] as CDR3 of SEQ ID NO:16.

In certain embodiments, the antigen-binding site is comprised within an antibody, e.g., a monoclonal antibody, for example, an antibody that comprises a heavy chain variable domain comprising the amino acid sequence of SEQ ID NO:15 and a light chain variable domain comprising the amino acid sequence of SEQ ID NO:16; or an antibody that comprises heavy chain CDRs 1-3 having the sequences of SEQ ID NOs: 63, 64, and 65, respectively, or SEQ ID NOs: 442, 64, and 443, respectively; and light chain CDRs 1-3 having the sequences of SEQ ID NOs: 66, 67, and 68, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANINTDG SEVYYVDSVKGRF TISRDNAKNSLYLQMNSLRAEDTAVYYCARDVGPGIAYQGHFD YWGQGTLVTVSS [SEQ ID NO:17]. In some embodiments, the antibody heavy chain variable domain is at least 95% identical to SEQ ID NO:17. In some embodiments, the heavy chain variable domain incorporates amino acid sequences FTFSSYWMS [SEQ ID NO:69] as CDR1, NINTDGSEVYYVDSVKG [SEQ ID NO:70] as CDR2, and ARDVGPGIAYQGHFDY [SEQ ID NO:71] as CDR3 of SEQ ID NO:17. In some embodiments, the heavy chain variable domain incorporates amino acid sequences SYWMS [SEQ ID NO:181] as CDR1, NINTDGSEVYYVDSVKG [SEQ ID NO:70] as CDR2, and DVGPGIAYQGHFDY [SEQ ID NO:444] as CDR3 of SEQ ID NO:17. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:17 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., 90%, 91%, 92%, 93%, 94%, 95%, 96%, 97%, 98%, 99%, or 100%) identical to the amino acid sequence DIQMTQSPSSLSASVGDRVTITCRASQVIYSYLNWYQQKPGKAPKLLIYAASSLKSGV PSRFSGSGSGTDFTLTISSLQPEDFATYYCQQVYDTPLTFGGGTKVEIK [SEQ ID NO:18]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:18, which includes amino acid sequences RASQVIYSYLN [SEQ ID NO:72] as CDR1, AASSLKS [SEQ ID NO:73] as CDR2, and QQVYDTPLT [SEQ ID NO:74] as CDR3 of SEQ ID NO:18.

In certain embodiments, the antigen-binding site is comprised within an antibody, e.g., a monoclonal antibody, for example, an antibody that comprises a heavy chain variable domain comprising the amino acid sequence of SEQ ID NO:17 and a light chain variable domain comprising the amino acid sequence of SEQ ID NO:18; or an antibody that comprises heavy chain CDRs 1-3 having the sequences of SEQ ID NOs: 69, 70, and 71, respectively, or SEQ ID NOs: 181, 70, and 444, respectively; and light chain CDRs 1-3 having the sequences of SEQ ID NOs: 72, 73, and 74, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of QLQLQESGPGLVKPSETLSLTCTVSGGSISSTDYYWGWIRQPPGKGLEWIGSIGYSGT YYNPSLKSRVTISVDTSKNQFSLKLSSVTAADTAVYYCARETAHDVHGMDVWGQG TTVTVSS [SEQ ID NO:19]. In some embodiments, the antibody heavy chain variable domain is at least 95% identical to SEQ ID NO:19. In some embodiments, the heavy chain variable domain incorporates amino acid sequences GSISSTDYYWG [SEQ ID NO:75] as CDR1, SIGYSGTYYNPSLKS [SEQ ID NO:76] as CDR2, and ARETAHDVHGMDV [SEQ ID NO:77] as CDR3 of SEQ ID NO:19. In some embodiments, the heavy chain variable domain incorporates amino acid sequences STDYYWG [SEQ ID NO:445] as CDR1, SIGYSGTYYNPSLKS [SEQ ID NO:76] as CDR2, and ETAHDVHGMDV [SEQ ID NO:446] as CDR3 of SEQ ID NO:19. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:19 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., 90%, 91%, 92%, 93%, 94%, 95%, 96%, 97%, 98%, 99%, or 100%) identical to the amino acid sequence EIVLTQSPATLSLSPGERATLSCRASHSVYSYLAWYQQKPGQAPRLLIYDASNRATGI PARFSGSGSGTDFTLTISSLEPEDFAVYYCQQYDNLPTFGGGTKVEIK [SEQ ID NO:20]. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:20, which includes amino acid sequences RASHSVYSYLA [SEQ ID NO:78] as CDR1, DASNRAT [SEQ ID NO:79] as CDR2, and QQYDNLPT [SEQ ID NO:80] as CDR3 of SEQ ID NO:20.

In certain embodiments, the antigen-binding site is comprised within an antibody, e.g., a monoclonal antibody, for example, an antibody that comprises a heavy chain variable domain comprising the amino acid sequence of SEQ ID NO:19 and a light chain variable domain comprising the amino acid sequence of SEQ ID NO:20; or an antibody that comprises heavy chain CDRs 1-3 having the sequences of SEQ ID NOs: 75, 76, and 77, respectively, or SEQ ID NOs: 445, 76, and 446, respectively; and light chain CDRs 1-3 having the sequences of SEQ ID NOs: 78, 79, and 80, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:266. In some embodiments, the antibody heavy chain variable domain is at least 95% identical to SEQ ID NO:266. In some embodiments, the heavy chain variable domain incorporates amino acid sequences SYAMS [SEQ ID NO:304] as CDR1, AISASGGSTYYADSVKG [SEQ ID NO:305] as CDR2, and PRAYYDSSGFKVNYGMDV [SEQ ID NO:306] as CDR3 of SEQ ID NO:266. In some embodiments, the heavy chain variable domain incorporates amino acid sequences FTFSSYAMS [SEQ ID NO:528] as CDR1, AISASGGSTYYADSVKG [SEQ ID NO:305] as CDR2, and ARPRAYYDSSGFKVNYGMDV [SEQ ID NO:529] as CDR3 of SEQ ID NO:266. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:266 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:267. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:267, which includes amino acid sequences RASQSVSSSFLA [SEQ ID NO:307] as CDR1, GASSRAT [SEQ ID NO:308] as CDR2, and QQASSSPPT [SEQ ID NO:309] as CDR3 of SEQ ID NO:267.

In certain embodiments, the antigen-binding site is comprised within an antibody, e.g., a monoclonal antibody, for example, an antibody that comprises a heavy chain variable domain comprising the amino acid sequence of SEQ ID NO:266 and a light chain variable domain comprising the amino acid sequence of SEQ ID NO:267; or an antibody that comprises heavy chain CDRs 1-3 having the sequences of SEQ ID NOs: 304, 305, and 306, respectively, or SEQ ID NOs: 528, 305, and 529, respectively; and light chain CDRs 1-3 having the sequences of SEQ ID NOs: 307, 308, and 309, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:268. In some embodiments, the antibody heavy chain variable domain is at least 95% identical to SEQ ID NO:268. In some embodiments, the heavy chain variable domain incorporates amino acid sequences SYAMS [SEQ ID NO:310] as CDR1, GISGSGGSTYYADSVKG [SEQ ID NO:311] as CDR2, and EGHSSSYYDHAFDI [SEQ ID NO:312] as CDR3 of SEQ ID NO:268. In some embodiments, the heavy chain variable domain incorporates amino acid sequences FTFSSYAMS [SEQ ID NO:530] as CDR1, GISGSGGSTYYADSVKG [SEQ ID NO:311] as CDR2, and AREGHSSSYYDHAFDI [SEQ ID NO:531] as CDR3 of SEQ ID NO:268. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:268 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:269. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:269, which includes amino acid sequences RASQSVSSDYLA [SEQ ID NO:313] as CDR1, GASSRAT [SEQ ID NO:314] as CDR2, and QQHSSAPPT [SEQ ID NO:315] as CDR3 of SEQ ID NO:269.

In certain embodiments, the antigen-binding site is comprised within an antibody, e.g., a monoclonal antibody, for example, an antibody that comprises a heavy chain variable domain comprising the amino acid sequence of SEQ ID NO:268 and a light chain variable domain comprising the amino acid sequence of SEQ ID NO:269; or an antibody that comprises heavy chain CDRs 1-3 having the sequences of SEQ ID NOs: 310, 311, and 312, respectively, or SEQ ID NOs: 530, 311, and 531, respectively; and light chain CDRs 1-3 having the sequences of SEQ ID NOs: 313, 314, and 315, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SYYWS [SEQ ID NO:316] as CDR1, SIYYSGSTNYNPSLKS [SEQ ID NO:317] as CDR2, and VGGVYSTIETYGMDV [SEQ ID NO:318] as CDR3 of SEQ ID NO:270. In some embodiments, the heavy chain variable domain incorporates amino acid sequences GSISSYYWS [SEQ ID NO:532] as CDR1, SIYYSGSTNYNPSLKS [SEQ ID NO:317] as CDR2, and ARVGGVYSTIETYGMDV [SEQ ID NO:533] as CDR3 of SEQ ID NO:270. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:271, which includes amino acid sequences RASQSVSSNLA [SEQ ID NO:319] as CDR1, GASTRAT [SEQ ID NO:320] as CDR2, and QQYTVYPPT [SEQ ID NO:321] as CDR3 of SEQ ID NO:271.

In certain embodiments, the antigen-binding site is comprised within an antibody, e.g., a monoclonal antibody, for example, an antibody that comprises a heavy chain variable domain comprising the amino acid sequence of SEQ ID NO:270 and a light chain variable domain comprising the amino acid sequence of SEQ ID NO:271; or an antibody that comprises heavy chain CDRs 1-3 having the sequences of SEQ ID NOs: 316, 317, and 318, respectively, or SEQ ID NOs: 532, 317, and 533, respectively; and light chain CDRs 1-3 having the sequences of SEQ ID NOs: 319, 320, and 321, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences GYYWS [SEQ ID NO:322] as CDR1, EIDHSGSTNYNPSLKS [SEQ ID NO:323] as CDR2, and QGIHGLRYFDL [SEQ ID NO:324] as CDR3 of SEQ ID NO:272. In some embodiments, the heavy chain variable domain incorporates amino acid sequences GSFSGYYWS [SEQ ID NO:534] as CDR1, EIDHSGSTNYNPSLKS [SEQ ID NO:323] as CDR2, and ARQGIHGLRYFDL [SEQ ID NO:535] as CDR3 of SEQ ID NO:272. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:273, which includes amino acid sequences RASQSVSSYLA [SEQ ID NO:325] as CDR1, DASNRAT [SEQ ID NO:326] as CDR2, and QQDHNFPYT [SEQ ID NO:327] as CDR3 of SEQ ID NO:273.

In certain embodiments, the antigen-binding site is comprised within an antibody, e.g., a monoclonal antibody, for example, an antibody that comprises a heavy chain variable domain comprising the amino acid sequence of SEQ ID NO:272 and a light chain variable domain comprising the amino acid sequence of SEQ ID NO:273; or an antibody that comprises heavy chain CDRs 1-3 having the sequences of SEQ ID NOs: 322, 323, and 324, respectively, or SEQ ID NOs: 534, 323, and 535, respectively; and light chain CDRs 1-3 having the sequences of SEQ ID NOs: 325, 326, and 327, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:274. In some embodiments, the antibody heavy chain variable domain is at least 95% identical to SEQ ID NO:274. In some embodiments, the heavy chain variable domain incorporates amino acid sequences SYWMS [SEQ ID NO:328] as CDR1, NINQDGSEKYYVDSVKG [SEQ ID NO:329] as CDR2, and EANYYGNVGDDY [SEQ ID NO:330] as CDR3 of SEQ ID NO:274. In some embodiments, the heavy chain variable domain incorporates amino acid sequences FTFSSYWMS [SEQ ID NO:536] as CDR1, NINQDGSEKYYVDSVKG [SEQ ID NO:329] as CDR2, and AREANYYGNVGDDY [SEQ ID NO:537] as CDR3 of SEQ ID NO:274. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:274 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:275. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:275, which includes amino acid sequences RASQSISSYLN [SEQ ID NO:331] as CDR1, AASSLQS [SEQ ID NO:332] as CDR2, and QQQYVTPIT [SEQ ID NO:333] as CDR3 of SEQ ID NO:275.

In certain embodiments, the antigen-binding site is comprised within an antibody, e.g., a monoclonal antibody, for example, an antibody that comprises a heavy chain variable domain comprising the amino acid sequence of SEQ ID NO:274 and a light chain variable domain comprising the amino acid sequence of SEQ ID NO:275; or an antibody that comprises heavy chain CDRs 1-3 having the sequences of SEQ ID NOs: 328, 329, and 330, respectively, or SEQ ID NOs: 536, 329, and 537, respectively; and light chain CDRs 1-3 having the sequences of SEQ ID NOs: 331, 332, and 333, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:276. In some embodiments, the antibody heavy chain variable domain is at least 95% identical to SEQ ID NO:276. In some embodiments, the heavy chain variable domain incorporates amino acid sequences SYWMS [SEQ ID NO:334] as CDR1, NINQDGSEKYYVDSVKG [SEQ ID NO:335] as CDR2, and EGGDSWYHAFDI [SEQ ID NO:336] as CDR3 of SEQ ID NO:276. In some embodiments, the heavy chain variable domain incorporates amino acid sequences FTFSSYWMS [SEQ ID NO:538] as CDR1, NINQDGSEKYYVDSVKG [SEQ ID NO:335] as CDR2, and AREGGDSWYHAFDI [SEQ ID NO:539] as CDR3 of SEQ ID NO:276. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:276 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:277. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:277, which includes amino acid sequences RASQGISSWLA [SEQ ID NO:337] as CDR1, AASNLQS [SEQ ID NO:338] as CDR2, and QQKLSLPLT [SEQ ID NO:339] as CDR3 of SEQ ID NO:277.

In certain embodiments, the antigen-binding site is comprised within an antibody, e.g., a monoclonal antibody, for example, an antibody that comprises a heavy chain variable domain comprising the amino acid sequence of SEQ ID NO:276 and a light chain variable domain comprising the amino acid sequence of SEQ ID NO:277; or an antibody that comprises heavy chain CDRs 1-3 having the sequences of SEQ ID NOs: 334, 335, and 336, respectively, or SEQ ID NOs: 538, 335, and 539, respectively; and light chain CDRs 1-3 having the sequences of SEQ ID NOs: 337, 338, and 339, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SGGYYWS [SEQ ID NO:340] as CDR1, SIYYSGSTYYNPSLKS [SEQ ID NO:341] as CDR2, and DRLDYSYNYGMDV [SEQ ID NO:342] as CDR3 of SEQ ID NO:278. In some embodiments, the heavy chain variable domain incorporates amino acid sequences GSISSGGYYWS [SEQ ID NO:540] as CDR1, SIYYSGSTYYNPSLKS [SEQ ID NO:341] as CDR2, and ARDRLDYSYNYGMDV [SEQ ID NO:541] as CDR3 of SEQ ID NO:278. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:279, which includes amino acid sequences RASQSISSYLN [SEQ ID NO:343] as CDR1, GASSLQS [SEQ ID NO:344] as CDR2, and QQVYSAPFT [SEQ ID NO:345] as CDR3 of SEQ ID NO:279.

In certain embodiments, the antigen-binding site is comprised within an antibody, e.g., a monoclonal antibody, for example, an antibody that comprises a heavy chain variable domain comprising the amino acid sequence of SEQ ID NO:278 and a light chain variable domain comprising the amino acid sequence of SEQ ID NO:279; or an antibody that comprises heavy chain CDRs 1-3 having the sequences of SEQ ID NOs: 340, 341, and 342, respectively, or SEQ ID NOs: 540, 341, and 541, respectively; and light chain CDRs 1-3 having the sequences of SEQ ID NOs: 343, 344, and 345, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SGYYWG [SEQ ID NO:346] as CDR1, SIYHSGSTNYNPSLKS [SEQ ID NO:347] as CDR2, and LPPWFGFSYFDL [SEQ ID NO:348] as CDR3 of SEQ ID NO:280. In some embodiments, the heavy chain variable domain incorporates amino acid sequences YSISSGYYWG [SEQ ID NO:542] as CDR1, SIYHSGSTNYNPSLKS [SEQ ID NO:347] as CDR2, and ARLPPWFGFSYFDL [SEQ ID NO:543] as CDR3 of SEQ ID NO:280. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:281, which includes amino acid sequences RASQSVSSYLA [SEQ ID NO:349] as CDR1, DASNRAT [SEQ ID NO:350] as CDR2, and QQVDNYPPT [SEQ ID NO:351] as CDR3 of SEQ ID NO:281.

In certain embodiments, the antigen-binding site is comprised within an antibody, e.g., a monoclonal antibody, for example, an antibody that comprises a heavy chain variable domain comprising the amino acid sequence of SEQ ID NO:280 and a light chain variable domain comprising the amino acid sequence of SEQ ID NO:281; or an antibody that comprises heavy chain CDRs 1-3 having the sequences of SEQ ID NOs: 346, 347, and 348, respectively, or SEQ ID NOs: 542, 347, and 543, respectively; and light chain CDRs 1-3 having the sequences of SEQ ID NOs: 349, 350, and 351, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SYWMS [SEQ ID NO:352] as CDR1, NIKQDGSEKYYVDSVKG [SEQ ID NO:353] as CDR2, and DVGPGIAYQGHFDY [SEQ ID NO:354] as CDR3 of SEQ ID NO:282. In some embodiments, the heavy chain variable domain incorporates amino acid sequences FTFSSYWMS [SEQ ID NO:544] as CDR1, NIKQDGSEKYYVDSVKG [SEQ ID NO:353] as CDR2, and ARDVGPGIAYQGHFDY [SEQ ID NO:545] as CDR3 of SEQ ID NO:282. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:283, which includes amino acid sequences RASQSISSYLN [SEQ ID NO:355] as CDR1, AASSLQS [SEQ ID NO:356] as CDR2, and QQVYDTPLT [SEQ ID NO:357] as CDR3 of SEQ ID NO:283.

In certain embodiments, the antigen-binding site is comprised within an antibody, e.g., a monoclonal antibody, for example, an antibody that comprises a heavy chain variable domain comprising the amino acid sequence of SEQ ID NO:282 and a light chain variable domain comprising the amino acid sequence of SEQ ID NO:283; or an antibody that comprises heavy chain CDRs 1-3 having the sequences of SEQ ID NOs: 352, 353, and 354, respectively, or SEQ ID NOs: 544, 353, and 545, respectively; and light chain CDRs 1-3 having the sequences of SEQ ID NOs: 355, 356, and 357, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SSSYYWG [SEQ ID NO:358] as CDR1, SIYYSGSTYYNPSLKS [SEQ ID NO:359] as CDR2, and ETAHDVHGMDV [SEQ ID NO:360] as CDR3 of SEQ ID NO:284. In some embodiments, the heavy chain variable domain incorporates amino acid sequences GSISSSSYYWG [SEQ ID NO:546] as CDR1, SIYYSGSTYYNPSLKS [SEQ ID NO:359] as CDR2, and ARETAHDVHGMDV [SEQ ID NO:547] as CDR3 of SEQ ID NO:284. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:285, which includes amino acid sequences RASQSVSSYLA [SEQ ID NO:361] as CDR1, DASNRAT [SEQ ID NO:362] as CDR2, and QQYDNLPT [SEQ ID NO:363] as CDR3 of SEQ ID NO:285.

In certain embodiments, the antigen-binding site is comprised within an antibody, e.g., a monoclonal antibody, for example, an antibody that comprises a heavy chain variable domain comprising the amino acid sequence of SEQ ID NO:284 and a light chain variable domain comprising the amino acid sequence of SEQ ID NO:285; or an antibody that comprises heavy chain CDRs 1-3 having the sequences of SEQ ID NOs: 358, 359, and 360, respectively, or SEQ ID NOs: 546, 359, and 547, respectively; and light chain CDRs 1-3 having the sequences of SEQ ID NOs: 361, 362, and 363, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:286. In some embodiments, the antibody heavy chain variable domain is at least 95% identical to SEQ ID NO:286. In some embodiments, the heavy chain variable domain incorporates amino acid sequences SYAIS [SEQ ID NO:364] as CDR1, SIIPIFGTANYAQKFQG [SEQ ID NO:365] as CDR2, and EVGYGWYTKIAFDI [SEQ ID NO:366] as CDR3 of SEQ ID NO:286. In some embodiments, the heavy chain variable domain incorporates amino acid sequences GTFSSYAIS [SEQ ID NO:548] as CDR1, SIIPIFGTANYAQKFQG [SEQ ID NO:365] as CDR2, and AREVGYGWYTKIAFDI [SEQ ID NO:549] as CDR3 of SEQ ID NO:286. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:286 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:287. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:287, which includes amino acid sequences RASQSVSSYLA [SEQ ID NO:367] as CDR1, DASKRAT [SEQ ID NO:368] as CDR2, and QQSSNHPST [SEQ ID NO:369] as CDR3 of SEQ ID NO:287.

In certain embodiments, the antigen-binding site is comprised within an antibody, e.g., a monoclonal antibody, for example, an antibody that comprises a heavy chain variable domain comprising the amino acid sequence of SEQ ID NO:286 and a light chain variable domain comprising the amino acid sequence of SEQ ID NO:287; or an antibody that comprises heavy chain CDRs 1-3 having the sequences of SEQ ID NOs: 364, 365, and 366, respectively, or SEQ ID NOs: 548, 365, and 549, respectively; and light chain CDRs 1-3 having the sequences of SEQ ID NOs: 367, 368, and 369, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SYYMH [SEQ ID NO:370] as CDR1, IINPSGGSTTYAQKFQG [SEQ ID NO:371] as CDR2, and EAADGFVGERYFDL [SEQ ID NO:372] as CDR3 of SEQ ID NO:288. In some embodiments, the heavy chain variable domain incorporates amino acid sequences YTFTSYYMH [SEQ ID NO:550] as CDR1, IINPSGGSTTYAQKFQG [SEQ ID NO:371] as CDR2, and AREAADGFVGERYFDL [SEQ ID NO:551] as CDR3 of SEQ ID NO:288. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:289, which includes amino acid sequences RSSQSLLHSNGYNYLD [SEQ ID NO:373] as CDR1, LGSNRAS [SEQ ID NO:374] as CDR2, and MQALGVPLT [SEQ ID NO:375] as CDR3 of SEQ ID NO:289.

In certain embodiments, the antigen-binding site is comprised within an antibody, e.g., a monoclonal antibody, for example, an antibody that comprises a heavy chain variable domain comprising the amino acid sequence of SEQ ID NO:288 and a light chain variable domain comprising the amino acid sequence of SEQ ID NO:289; or an antibody that comprises heavy chain CDRs 1-3 having the sequences of SEQ ID NOs: 370, 371, and 372, respectively, or SEQ ID NOs: 550, 371, and 551, respectively; and light chain CDRs 1-3 having the sequences of SEQ ID NOs: 373, 374, and 375, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:290. In some embodiments, the antibody heavy chain variable domain is at least 95% identical to SEQ ID NO:290. In some embodiments, the heavy chain variable domain incorporates amino acid sequences GYYMH [SEQ ID NO:376] as CDR1, MINPYGGSTRYAQKFQG [SEQ ID NO:377] as CDR2, and EAADGFVGERYFDL [SEQ ID NO:378] as CDR3 of SEQ ID NO:290. In some embodiments, the heavy chain variable domain incorporates amino acid sequences YTFSGYYMH [SEQ ID NO:552] as CDR1, MINPYGGSTRYAQKFQG [SEQ ID NO:377] as CDR2, and AREAADGFVGERYFDL [SEQ ID NO:553] as CDR3 of SEQ ID NO:290. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:290 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:291. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:291, which includes amino acid sequences RSSQSLLYSNGYNYLD [SEQ ID NO:379] as CDR1, LGSNRAS [SEQ ID NO:380] as CDR2, and MQDVALPIT [SEQ ID NO:381] as CDR3 of SEQ ID NO:291.

In certain embodiments, the antigen-binding site is comprised within an antibody, e.g., a monoclonal antibody, for example, an antibody that comprises a heavy chain variable domain comprising the amino acid sequence of SEQ ID NO:290 and a light chain variable domain comprising the amino acid sequence of SEQ ID NO:291; or an antibody that comprises heavy chain CDRs 1-3 having the sequences of SEQ ID NOs: 376, 377, and 378, respectively, or SEQ ID NOs: 552, 377, and 553, respectively; and light chain CDRs 1-3 having the sequences of SEQ ID NOs: 379, 380, and 381, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:292. In some embodiments, the antibody heavy chain variable domain is at least 95% identical to SEQ ID NO:292. In some embodiments, the heavy chain variable domain incorporates amino acid sequences IYYMH [SEQ ID NO:382] as CDR1, IINPSSGSTVYAQKFQG [SEQ ID NO:383] as CDR2, and GAGYDDEDMDV [SEQ ID NO:384] as CDR3 of SEQ ID NO:292. In some embodiments, the heavy chain variable domain incorporates amino acid sequences YTFEIYYMH [SEQ ID NO:554] as CDR1, IINPSSGSTVYAQKFQG [SEQ ID NO:383] as CDR2, and ARGAGYDDEDMDV [SEQ ID NO:555] as CDR3 of SEQ ID NO:292. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:292 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:293. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:293, which includes amino acid sequences RASQGIDSWLA [SEQ ID NO:385] as CDR1, AASSLQS [SEQ ID NO:386] as CDR2, and QQAHSYPLT [SEQ ID NO:387] as CDR3 of SEQ ID NO:293.

In certain embodiments, the antigen-binding site is comprised within an antibody, e.g., a monoclonal antibody, for example, an antibody that comprises a heavy chain variable domain comprising the amino acid sequence of SEQ ID NO:292 and a light chain variable domain comprising the amino acid sequence of SEQ ID NO:293; or an antibody that comprises heavy chain CDRs 1-3 having the sequences of SEQ ID NOs: 382, 383, and 384, respectively, or SEQ ID NOs: 554, 383, and 555, respectively; and light chain CDRs 1-3 having the sequences of SEQ ID NOs: 385, 386, and 387, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:294. In some embodiments, the antibody heavy chain variable domain is at least 95% identical to SEQ ID NO:294. In some embodiments, the heavy chain variable domain incorporates amino acid sequences GYWMS [SEQ ID NO:388] as CDR1, NINQDGSEEYYVDSVKG [SEQ ID NO:389] as CDR2, and EANYYGNVGDDY [SEQ ID NO:390] as CDR3 of SEQ ID NO:294. In some embodiments, the heavy chain variable domain incorporates amino acid sequences FTFGGYWMS [SEQ ID NO:556] as CDR1, NINQDGSEEYYVDSVKG [SEQ ID NO:389] as CDR2, and AREANYYGNVGDDY [SEQ ID NO:557] as CDR3 of SEQ ID NO:294. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:294 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:295. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:295, which includes amino acid sequences RASQSIYNYLN [SEQ ID NO:391] as CDR1, AASNLHS [SEQ ID NO:392] as CDR2, and QQAFHVPIT [SEQ ID NO:393] as CDR3 of SEQ ID NO:295.

In certain embodiments, the antigen-binding site is comprised within an antibody, e.g., a monoclonal antibody, for example, an antibody that comprises a heavy chain variable domain comprising the amino acid sequence of SEQ ID NO:294 and a light chain variable domain comprising the amino acid sequence of SEQ ID NO:295; or an antibody that comprises heavy chain CDRs 1-3 having the sequences of SEQ ID NOs: 388, 389, and 390, respectively, or SEQ ID NOs: 556, 389, and 557, respectively; and light chain CDRs 1-3 having the sequences of SEQ ID NOs: 391, 392, and 393, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:296. In some embodiments, the antibody heavy chain variable domain is at least 95% identical to SEQ ID NO:296. In some embodiments, the heavy chain variable domain incorporates amino acid sequences GYWMS [SEQ ID NO:394] as CDR1, NINQDGSEVYYVDSVKG [SEQ ID NO:395] as CDR2, and EANYYGNVGDDY [SEQ ID NO:396] as CDR3 of SEQ ID NO:296. In some embodiments, the heavy chain variable domain incorporates amino acid sequences FTFPGYWMS [SEQ ID NO:558] as CDR1, NINQDGSEVYYVDSVKG [SEQ ID NO:395] as CDR2, and AREANYYGNVGDDY [SEQ ID NO:559] as CDR3 of SEQ ID NO:296. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:296 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:297. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:297, which includes amino acid sequences RASQSIYNYLN [SEQ ID NO:397] as CDR1, AASSTQS [SEQ ID NO:398] as CDR2, and QQAFHVPIT [SEQ ID NO:399] as CDR3 of SEQ ID NO:297.

In certain embodiments, the antigen-binding site is comprised within an antibody, e.g., a monoclonal antibody, for example, an antibody that comprises a heavy chain variable domain comprising the amino acid sequence of SEQ ID NO:296 and a light chain variable domain comprising the amino acid sequence of SEQ ID NO:297; or an antibody that comprises heavy chain CDRs 1-3 having the sequences of SEQ ID NOs: 394, 395, and 396, respectively, or SEQ ID NOs: 558, 395, and 559, respectively; and light chain CDRs 1-3 having the sequences of SEQ ID NOs: 397, 398, and 399, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:298. In some embodiments, the antibody heavy chain variable domain is at least 95% identical to SEQ ID NO:298. In some embodiments, the heavy chain variable domain incorporates amino acid sequences SYWMS [SEQ ID NO:400] as CDR1, NINQDGSEVYYVDSVKG [SEQ ID NO:401] as CDR2, and DVGPGIAYQGHFDY [SEQ ID NO:402] as CDR3 of SEQ ID NO:298. In some embodiments, the heavy chain variable domain incorporates amino acid sequences FTFSSYWMS [SEQ ID NO:560] as CDR1, NINQDGSEVYYVDSVKG [SEQ ID NO:401] as CDR2, and ARDVGPGIAYQGHFDY [SEQ ID NO:561] as CDR3 of SEQ ID NO:298. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:298 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:299. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:299, which includes amino acid sequences RASQSIYYYLN [SEQ ID NO:403] as CDR1, AASSRQS [SEQ ID NO:404] as CDR2, and QQVYDTPLT [SEQ ID NO:405] as CDR3 of SEQ ID NO:299.

In certain embodiments, the antigen-binding site is comprised within an antibody, e.g., a monoclonal antibody, for example, an antibody that comprises a heavy chain variable domain comprising the amino acid sequence of SEQ ID NO:298 and a light chain variable domain comprising the amino acid sequence of SEQ ID NO:299; or an antibody that comprises heavy chain CDRs 1-3 having the sequences of SEQ ID NOs: 400, 401, and 402, respectively, or SEQ ID NOs: 560, 401, and 561, respectively; and light chain CDRs 1-3 having the sequences of SEQ ID NOs: 403, 404, and 405, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:300. In some embodiments, the antibody heavy chain variable domain is at least 95% identical to SEQ ID NO:300. In some embodiments, the heavy chain variable domain incorporates amino acid sequences NYYMH [SEQ ID NO:406] as CDR1, WINPFSGGTRYAQKFQG [SEQ ID NO:407] as CDR2, and DVGSSAYYYMDV [SEQ ID NO:408] as CDR3 of SEQ ID NO:300. In some embodiments, the heavy chain variable domain incorporates amino acid sequences YTFSNYYMH [SEQ ID NO:562] as CDR1, WINPFSGGTRYAQKFQG [SEQ ID NO:407] as CDR2, and ARDVGSSAYYYMDV [SEQ ID NO:563] as CDR3 of SEQ ID NO:300. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:300 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:301. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:301, which includes amino acid sequences EASKGISSWLA [SEQ ID NO:409] as CDR1, AASDLQS [SEQ ID NO:410] as CDR2, and QQAFLFPPT [SEQ ID NO:411] as CDR3 of SEQ ID NO:301.

In certain embodiments, the antigen-binding site is comprised within an antibody, e.g., a monoclonal antibody, for example, an antibody that comprises a heavy chain variable domain comprising the amino acid sequence of SEQ ID NO:300 and a light chain variable domain comprising the amino acid sequence of SEQ ID NO:301; or an antibody that comprises heavy chain CDRs 1-3 having the sequences of SEQ ID NOs: 406, 407, and 408, respectively, or SEQ ID NOs: 562, 407, and 563, respectively; and light chain CDRs 1-3 having the sequences of SEQ ID NOs: 409, 410, and 411, respectively.

In certain embodiments, the present invention provides an antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:302. In some embodiments, the antibody heavy chain variable domain is at least 95% identical to SEQ ID NO:302. In some embodiments, the heavy chain variable domain incorporates amino acid sequences SYWIG [SEQ ID NO:412] as CDR1, SIYPGDSDTRYSPSFQG [SEQ ID NO:413] as CDR2, and ELAYGDYKGGVDY [SEQ ID NO:414] as CDR3 of SEQ ID NO:302. In some embodiments, the heavy chain variable domain incorporates amino acid sequences YSFTSYWIG [SEQ ID NO:564] as CDR1, SIYPGDSDTRYSPSFQG [SEQ ID NO:413] as CDR2, and ARELAYGDYKGGVDY [SEQ ID NO:565] as CDR3 of SEQ ID NO:302. In certain embodiments, the antibody heavy chain variable domain that includes an amino acid sequence at least 90% (e.g., 90%, 91%, 92%, 93%, 94%, 95%, 96%, 97%, 98%, 99%, or 100%) identical to the amino acid sequence of SEQ ID NO:302 is combined with a light chain variable domain to form an antigen-binding site capable of binding to CD33. For example, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:303. In certain embodiments, an antibody heavy chain variable domain at least 90% (e.g., 90%, 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., 90%, 91%, 92%, 93%, 94%, 95%, 96%, 97%, 98%, 99%, or 100%) identical to the amino acid sequence of SEQ ID NO:303, which includes amino acid sequences RASQSVSSSFLA [SEQ ID NO:415] as CDR1, GASSRAT [SEQ ID NO:416] as CDR2, and QQLDSPPPT [SEQ ID NO:417] as CDR3 of SEQ ID NO:303.

In certain embodiments, the antigen-binding site is comprised within an antibody, e.g., a monoclonal antibody, for example, an antibody that comprises a heavy chain variable domain comprising the amino acid sequence of SEQ ID NO:302 and a light chain variable domain comprising the amino acid sequence of SEQ ID NO:303; or an antibody that comprises heavy chain CDRs 1-3 having the sequences of SEQ ID NOs: 412, 413, and 414, respectively, or SEQ ID NOs: 564, 413, and 565, respectively; and light chain CDRs 1-3 having the sequences of SEQ ID NOs: 415, 416, and 417, respectively.

An antibody heavy chain variable domain of SEQ ID NOs: 1, 3, 5, 7, 9, 11, 13, 15, 17, 19, 266, 268, 270, 272, 274, 276, 278, 280, 282, 284, 286, 288, 290, 292, 294, 296, 298, 300, and/or 302 can optionally be coupled to an amino acid sequence at least 90% (e.g., 90%, 91%, 92%, 93%, 94%, 95%, 96%, 97%, 98%, 99%, or 100%) identical to an antibody constant region, such as an IgG constant region including hinge, CH2 and CH3 domains with or without CH1 domain. In some embodiments, the amino acid sequence of the constant region is at least 90% (e.g., 90%, 91%, 92%, 93%, 94%, 95%, 96%, 97%, 98%, 99%, or 100%) identical to an antibody constant region, such as a human antibody constant region, a human IgG1 constant region, a human IgG2 constant region, a human IgG3 constant region, or a human IgG4 constant region. In some other embodiments, the amino acid sequence of the constant region is at least 90% (e.g., 90%, 91%, 92%, 93%, 94%, 95%, 96%, 97%, 98%, 99%, or 100%) identical to an antibody constant region from another mammal, such as rabbit, dog, cat, mouse, or horse. One or more mutations can be incorporated into the constant region as compared to a human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the immunoglobulin heavy chain variable region comprises the amino acid sequence of SEQ ID NO:1 (Ab1-V_{H}), and the immunoglobulin light chain variable region comprises the amino acid sequence of SEQ ID NO:2 (Ab1-V_{L}).

In certain embodiments, the immunoglobulin heavy chain variable region comprises the amino acid sequence of SEQ ID NO:3 (Ab2-V_{H}), and the immunoglobulin light chain variable region comprises the amino acid sequence of SEQ ID NO:4 (Ab2-V_{L}).

In certain embodiments, the immunoglobulin heavy chain variable region comprises the amino acid sequence of SEQ ID NO:5 (Ab3-V_{H}), and the immunoglobulin light chain variable region comprises the amino acid sequence of SEQ ID NO:6 (Ab3-V_{L}).

In certain embodiments, the immunoglobulin heavy chain variable region comprises the amino acid sequence of SEQ ID NO:7 (Ab4-V_{H}), and the immunoglobulin light chain variable region comprises the amino acid sequence of SEQ ID NO:8 (Ab4-V_{L}).

In certain embodiments, the immunoglobulin heavy chain variable region comprises the amino acid sequence of SEQ ID NO:9 (Ab5-V_{H}), and the immunoglobulin light chain variable region comprises the amino acid sequence of SEQ ID NO:10 (Ab5-V_{L}).

In certain embodiments, the immunoglobulin heavy chain variable region comprises the amino acid sequence of SEQ ID NO:11 (Ab6-V_{H}), and the immunoglobulin light chain variable region comprises the amino acid sequence of SEQ ID NO:12 (Ab6-V_{L}).

In certain embodiments, the immunoglobulin heavy chain variable region comprises the amino acid sequence of SEQ ID NO:13 (Ab7-V_{H}), and the immunoglobulin light chain variable region comprises the amino acid sequence of SEQ ID NO:14 (Ab7-V_{L}).

In certain embodiments, the immunoglobulin heavy chain variable region comprises the amino acid sequence of SEQ ID NO:15 (Ab8-V_{H}), and the immunoglobulin light chain variable region comprises the amino acid sequence of SEQ ID NO:16 (Ab8-V_{L}).

In certain embodiments, the immunoglobulin heavy chain variable region comprises the amino acid sequence of SEQ ID NO:17 (Ab9-V_{H}), and the immunoglobulin light chain variable region comprises the amino acid sequence of SEQ ID NO:18 (Ab9-V_{L}).

In certain embodiments, the immunoglobulin heavy chain variable region comprises the amino acid sequence of SEQ ID NO:19 (Ab10-V_{H}), and the immunoglobulin light chain variable region comprises the amino acid sequence of SEQ ID NO:20 (Ab10-V_{L}).

In certain embodiments, the immunoglobulin heavy chain variable region comprises the amino acid sequence of SEQ ID NO:266 (Ab11-V_{H}), and the immunoglobulin light chain variable region comprises the amino acid sequence of SEQ ID NO:267 (Ab11-V_{L}).

In certain embodiments, the immunoglobulin heavy chain variable region comprises the amino acid sequence of SEQ ID NO:268 (Ab12-V_{H}), and the immunoglobulin light chain variable region comprises the amino acid sequence of SEQ ID NO:269 (Ab12-V_{L}).

In certain embodiments, the immunoglobulin heavy chain variable region comprises the amino acid sequence of SEQ ID NO:270 (Ab13-V_{H}), and the immunoglobulin light chain variable region comprises the amino acid sequence of SEQ ID NO:271 (Ab13-V_{L}).

In certain embodiments, the immunoglobulin heavy chain variable region comprises the amino acid sequence of SEQ ID NO:272 (Ab14-V_{H}), and the immunoglobulin light chain variable region comprises the amino acid sequence of SEQ ID NO:273 (Ab14-V_{L}).

In certain embodiments, the immunoglobulin heavy chain variable region comprises the amino acid sequence of SEQ ID NO:274 (Ab15-V_{H}), and the immunoglobulin light chain variable region comprises the amino acid sequence of SEQ ID NO:275 (Ab15-V_{L}).

In certain embodiments, the immunoglobulin heavy chain variable region comprises the amino acid sequence of SEQ ID NO:276 (Ab16-V_{H}), and the immunoglobulin light chain variable region comprises the amino acid sequence of SEQ ID NO:277 (Ab16-V_{L}).

In certain embodiments, the immunoglobulin heavy chain variable region comprises the amino acid sequence of SEQ ID NO:278 (Ab17-V_{H}), and the immunoglobulin light chain variable region comprises the amino acid sequence of SEQ ID NO:279 (Ab17-V_{L}).

In certain embodiments, the immunoglobulin heavy chain variable region comprises the amino acid sequence of SEQ ID NO:280 (Ab18-V_{H}), and the immunoglobulin light chain variable region comprises the amino acid sequence of SEQ ID NO:281 (Ab18-V_{L}).

In certain embodiments, the immunoglobulin heavy chain variable region comprises the amino acid sequence of SEQ ID NO:282 (Ab19-V_{H}), and the immunoglobulin light chain variable region comprises the amino acid sequence of SEQ ID NO:283 (Ab19-V_{L}).

In certain embodiments, the immunoglobulin heavy chain variable region comprises the amino acid sequence of SEQ ID NO:284 (Ab20-V_{H}), and the immunoglobulin light chain variable region comprises the amino acid sequence of SEQ ID NO:285 (Ab20-V_{L}).

In certain embodiments, the immunoglobulin heavy chain variable region comprises the amino acid sequence of SEQ ID NO:286 (Ab21-V_{H}), and the immunoglobulin light chain variable region comprises the amino acid sequence of SEQ ID NO:287 (Ab21-V_{L}).

In certain embodiments, the immunoglobulin heavy chain variable region comprises the amino acid sequence of SEQ ID NO:288 (Ab22-V_{H}), and the immunoglobulin light chain variable region comprises the amino acid sequence of SEQ ID NO:289 (Ab22-V_{L}).

In certain embodiments, the immunoglobulin heavy chain variable region comprises the amino acid sequence of SEQ ID NO:290 (Ab23-V_{H}), and the immunoglobulin light chain variable region comprises the amino acid sequence of SEQ ID NO:291 (Ab23-V_{L}).

In certain embodiments, the immunoglobulin heavy chain variable region comprises the amino acid sequence of SEQ ID NO:292 (Ab24-V_{H}), and the immunoglobulin light chain variable region comprises the amino acid sequence of SEQ ID NO:293 (Ab24-V_{L}).

In certain embodiments, the immunoglobulin heavy chain variable region comprises the amino acid sequence of SEQ ID NO:294 (Ab25-V_{H}), and the immunoglobulin light chain variable region comprises the amino acid sequence of SEQ ID NO:295 (Ab25-V_{L}).

In certain embodiments, the immunoglobulin heavy chain variable region comprises the amino acid sequence of SEQ ID NO:296 (Ab26-V_{H}), and the immunoglobulin light chain variable region comprises the amino acid sequence of SEQ ID NO:297 (Ab26-V_{L}).

In certain embodiments, the immunoglobulin heavy chain variable region comprises the amino acid sequence of SEQ ID NO:298 (Ab27-V_{H}), and the immunoglobulin light chain variable region comprises the amino acid sequence of SEQ ID NO:299 (Ab27-V_{L}).

In certain embodiments, the immunoglobulin heavy chain variable region comprises the amino acid sequence of SEQ ID NO:300 (Ab28-V_{H}), and the immunoglobulin light chain variable region comprises the amino acid sequence of SEQ ID NO:301 (Ab28-V_{L}).

In certain embodiments, the immunoglobulin heavy chain variable region comprises the amino acid sequence of SEQ ID NO:302 (Ab29-V_{H}), and the immunoglobulin light chain variable region comprises the amino acid sequence of SEQ ID NO:303 (Ab29-V_{L}).

In certain embodiments, the present invention provides a protein that includes a human CD33 antigen-binding site including a heavy chain variable domain, which includes an amino acid sequence at least 90% (e.g., 90%, 91%, 92%, 93%, 94%, 95%, 96%, 97%, 98%, 99%, or 100%) identical to the amino acid sequence of SEQ ID NO: 1, 3, 5, 7, 9, 11, 13, 15, 17, 19, 266, 268, 270, 272, 274, 276, 278, 280, 282, 284, 286, 288, 290, 292, 294, 296, 298, 300, or 302, and further comprises a second antigen binding site same or different from the antigen-binding site that binds to human CD33.

In certain embodiments, any of the foregoing isolated antibodies has a K_{D} of 1 nM or lower, 5 nM or lower, or 12 nM or lower for extracellular domain of human CD33, as measured by surface plasmon resonance (SPR) (e.g., using the Biacore method described in Example 1 infra) or by bio-layer interferometry (BLI) (e.g., using the Octet method described in Example 1 infra), and/or binds CD33 from a body fluid, tissue, and/or cell of a subject. In certain embodiments, any of the foregoing isolated antibodies has a K_{d} (i.e., off-rate, also called K_{off}) equal to or lower than 1×10⁻⁵, 1×10⁻⁴, 1×10⁻³, 5×10⁻³, 0.01, 0.02, or 0.05 l/s, as measured by SPR (e.g., using the Biacore method described in Example 1 infra) or by BLI (e.g., using the Octet method described in Example 1 infra).

In specific embodiments of any of the antibodies or binding fragments thereof, provided herein, the antibody is a monoclonal antibody, a chimeric antibody, a diabody, a Fab fragment, a Fab′ fragment, or F(ab′)2 fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (kih) IgG with common light chain, crossmab, ortho-Fab IgG, DVD-Ig, 2 in 1-IgG, IgG-scFv, sdFv2-Fc, bi-nanobody, tandAb, dual-affinity retargeting antibody (DART), DART-Fc, scFv-HSA-scFv (where HSA=human serum albumin), or dock-and-lock (DNL)-Fab3.

In another aspect, the invention provides one or more isolated nucleic acids comprising sequences encoding an immunoglobulin heavy chain and/or immunoglobulin light chain variable region of any one of the foregoing antibodies. The invention provides one or more expression vectors that express the immunoglobulin heavy chain and/or immunoglobulin light chain variable region of any one of the foregoing antibodies. Similarly the invention provides host cells comprising one or more of the foregoing expression vectors and/or isolated nucleic acids.

Formulations including any of the proteins that include a CD33-binding domain described herein and methods of enhancing tumor cell death using these proteins and/or formulations are also provided.

In another aspect, the invention provides a method of treating a cancer, for example, a CD33-associated cancer, in a subject. The method comprises administering to the subject an effective amount of a protein containing any CD33-binding domain described herein, for example, an anti-CD33 antibody, to treat the cancer in the subject.

In another aspect, the invention provides a method of inhibiting cancer growth, for example, the growth of a CD33-associated cancer, in a subject. The method comprises exposing the subject to an effective amount of a protein containing any CD33-binding domain described herein, for example, an anti-CD33 antibody, to inhibit cancer growth in the subject.

Another aspect of the invention provides a method of treating cancer in a patient. The method comprises administering to a patient in need thereof a therapeutically effective amount of a protein containing any CD33-binding domain described herein. Exemplary cancers for treatment using the protein include, for example, wherein the cancer is selected from the group consisting of AML, myelodysplastic syndromes, chronic myelomonocytic leukemia, myeloid blast crisis of chronic myeloid leukemia, and ALLs.

In certain embodiments, the present invention provides a protein disclosed herein comprising a sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, and SEQ ID NO:488.

In certain embodiments, the present invention provides a protein disclosed herein comprising an scFv linked to an antibody Fc domain, wherein the scFv linked to the antibody Fc domain is represented by a sequence selected from SEQ ID NO:187, SEQ ID NO:197, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, and SEQ ID NO:243.

In certain embodiments, the present invention provides a protein disclosed herein comprising a sequence selected from SEQ ID NO:189, SEQ ID NO:196, SEQ ID NO:244, and SEQ ID NO:245.

In certain embodiments, the present invention provides a protein disclosed herein comprising a sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides a protein disclosed herein comprising a sequence at least 95% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides a protein disclosed herein comprising a sequence at least 99% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484.

In certain embodiments, the present invention provides a protein disclosed herein comprising a sequence at least 90% (e.g., 90%, 91%, 92%, 93%, 94%, 95%, 96%, 97%, 98%, 99%, or 100%) identical to an amino acid sequence selected from SEQ ID NO:187, SEQ ID NO:197, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, and SEQ ID NO:243. In certain embodiments, the present invention provides a protein disclosed herein comprising a sequence at least 95% identical to an amino acid sequence selected from SEQ ID NO:187, SEQ ID NO:197, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, and SEQ ID NO:243. In certain embodiments, the present invention provides a protein disclosed herein comprising a sequence at least 99% identical to an amino acid sequence selected from SEQ ID NO:187, SEQ ID NO:197, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, and SEQ ID NO:243.

Another aspect of the present invention provides a formulation comprising a protein as disclosed herein, and a pharmaceutically acceptable carrier.

Another aspect of the present invention provides a nucleic acid encoding a chimeric antigen receptor (CAR), wherein the nucleic acid comprises a nucleic acid sequence that encodes a CD33-binding scFv comprising a sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484; a nucleic acid sequence encoding a transmembrane domain; and a nucleic acid sequence encoding an intracellular signaling domain.

Another aspect of the present invention provides a CAR comprising a CD33-binding scFv comprising a sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484; a transmembrane domain; and an intracellular signaling domain.

In certain embodiments of the nucleic acid or the CAR, the CD33-binding scFv comprises a sequence at least 95% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the CD33-binding scFv comprises a sequence at least 99% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the CD33-binding scFv comprises an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484.

In certain embodiments, the transmembrane domain is selected from the transmembrane regions of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD152, and CD154. In certain embodiments, the CD33-binding scFv is connected to the transmembrane domain by a hinge region.

In certain embodiments, the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In certain embodiments, the intracellular signaling domain further comprises a costimulatory signaling domain comprising a functional signaling domain of a costimulatory receptor. In certain embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

Another aspect of the present invention provides a vector comprising the nucleic acid. In certain embodiments, the vector is a viral vector (e.g., AAV vector, lentiviral vector, or adenoviral vector).

Another aspect of the present invention provides an immune effector cell expressing the CAR as disclosed herein. In certain embodiments, the CAR is expressed on the plasma membrane of the cell. Another aspect of the present invention provides an immune effector cell comprising the nucleic acid encoding the CAR as disclosed herein. Also provided is an immune effector cell comprising the vector that comprises the nucleic acid. In certain embodiments, the immune effector cell is a T cell (e.g., CD8⁺ T cell, CD4⁺ T cell, or NKT cell). In certain embodiments, the effector cell is an NK cell.

Another aspect of the present invention provides a CD33/CD3-directed bispecific T-cell engager comprising a protein comprising a sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides a CD33/CD3-directed bispecific T-cell engager comprising a protein comprising a sequence at least 95% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides a CD33/CD3-directed bispecific T-cell engager comprising a protein comprising a sequence at least 99% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides a CD33/CD3-directed bispecific T-cell engager comprising a protein comprising an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484.

Another aspect of the present invention provides an antibody-drug conjugate comprising a protein comprising a sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides an antibody-drug conjugate comprising a protein comprising a sequence at least 95% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides an antibody-drug conjugate comprising a protein comprising a sequence at least 99% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the present invention provides an antibody-drug conjugate comprising a protein comprising an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the antibody-drug conjugate further comprises a drug moiety selected from auristatin, N-acetyl-γ calicheamicin, maytansinoid, pyrrolobenzodiazepine, and SN-38.

Another aspect of the present invention provides an immunocytokine comprising a sequence at least 90% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484 connected to a cytokine. In certain embodiments, the present invention provides an immunocytokine comprising a sequence at least 95% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484 connected to a cytokine. In certain embodiments, the present invention provides an immunocytokine comprising a sequence at least 99% identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484 connected to a cytokine. In certain embodiments, the present invention provides an immunocytokine comprising an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484 connected to a cytokine. In certain embodiments, the cytokine is selected from IL-2, IL-4, IL-10, IL-12, IL-15, TNF, and IFNα.

Another aspect of the present invention provides a method of treating a CD33-expressing cancer, the method comprising administering a therapeutically effective amount of a protein or formulation thereof disclosed herein to a subject in need thereof. In certain embodiments, the method comprises administering a therapeutically effective amount of an antibody or formulation thereof to a subject in need thereof. In certain embodiments, the method comprises administering a therapeutically effective amount of a monoclonal antibody or formulation thereof to a subject in need thereof. In certain embodiments, the method comprises administering a therapeutically effective amount of an engager or formulation thereof to a subject in need thereof.

In certain embodiments, the cancer is selected from the group consisting of acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma. In certain embodiments, the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN). In certain embodiments, the AML is characterized by expression of CLL-1 on the AML leukemia stem cells (LSCs). In certain embodiments, the LSCs further express a membrane marker selected from CD34, CD38, CD123, TIM3, CD25, CD32, and CD96.

In certain embodiments, the AML is a minimal residual disease (MRD). In certain embodiments, the MRD is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)). In certain embodiments, the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U). In certain embodiments, the MDS is a primary MDS or a secondary MDS.

In certain embodiments, the ALL is selected from B-cell acute lymphoblastic leukemia (B-ALL) and T-cell acute lymphoblastic leukemia (T-ALL). In certain embodiments, the MPN is selected from polycythaemia vera, essential thrombocythemia (ET), and myelofibrosis. In certain embodiments, the non-Hodgkin lymphoma is selected from B-cell lymphoma and T-cell lymphoma. In certain embodiments, the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.

These and other aspects and advantages of the invention are illustrated by the following figures, detailed description and claims.

### DESCRIPTION OF THE DRAWINGS

The invention can be more completely understood with reference to the following drawings.

FIG. 1 shows a structural representation of the extracellular domain of human CD33 extracellular domain (ECD). CD33 ECD contains two prominent domains: distal V domain and membrane proximal C domain. Ligand binding interface is located on the V domain. Function of the C domain is unknown. ECD of CD33 is heavily glycosylated, with 2 N-linked glycosylation sites located in the V domain and 3 N-linked glycosylation sites located in the C domain. ECD of human CD33 contains several SNPs, with most prominent mutation R69G that is found in 42% patients. SNP R69G is in the V domain.

FIG. 2 shows an alignment of the primary sequences of full length human and cyno CD33 (SEQ ID NO:598 and SEQ ID NO:599, respectively). V domain is underlined in blue, C domain is underlined in green. Difference in sequences are framed in red.

FIGS. 3A-3K show SPR profiles of Fab fragments from CD33 monoclonal antibodies binding to human CD33 ECD measured by Biacore at 37° C. Each Fab fragment includes a CD33-binding clone described herein. FIG. 3A is a Biacore profile of ADI-10159; FIG. 3B is a Biacore profile of ADI-10177; FIG. 3C is a Biacore profile of ADI-11776; FIG. 3D is a Biacore profile of ADI-11801; FIG. 3E is a Biacore profile of ADI-11807; FIG. 3F is a Biacore profile of ADI-11809; FIG. 3G is a Biacore profile of ADI-11815; FIG. 3H is a Biacore profile of ADI-11819; FIG. 3I is a Biacore profile of ADI-11830; FIG. 3J is a Biacore profile of ADI-11835; and FIG. 3K is a Biacore profile of Fab fragment from Lintuzumab.

FIGS. 4A-4H show SPR profiles of Fab fragments from CD33 monoclonal antibodies binding to cyno CD33 ECD measured by Biacore at 37° C. Each Fab fragment includes a CD33-binding clone described herein. FIG. 4A is a Biacore profile of ADI-10159; FIG. 4B is a Biacore profile of ADI-10177; FIG. 4C is a Biacore profile of ADI-11776; FIG. 4D is a Biacore profile of ADI-11807; FIG. 4E is a Biacore profile of ADI-11809; FIG. 4F is a Biacore profile of ADI-11819; FIG. 4G is a Biacore profile of ADI-11830; and FIG. 4H is a Biacore profile of ADI-11835.

FIGS. 5A-5T show SPR profiles of FABs from CD33 monoclonal antibodies binding to V domain and C domain of human CD33 measured at 37° C. Each Fab fragment includes a CD33-binding clone described herein. FIGS. 5A-5J represent binding to the V-domain; panels K-T represent binding to the C domain. FIGS. 5A and 5K are Biacore profiles of ADI-10159; FIGS. 5B and 5L are Biacore profiles of ADI-10177; FIGS. 5C and 5M are Biacore profiles of ADI-11776; FIGS. 5D and 5N are Biacore profiles of ADI-11801; FIGS. 5E and 5O are Biacore profiles of ADI-11807; FIGS. 5F and 5P are Biacore profiles of ADI-11809; FIGS. 5G and 5Q are Biacore profiles of ADI-11815; FIGS. 5H and 5R are Biacore profiles of ADI-11819; FIGS. 5I and 5S are Biacore profiles of ADI-11830; and FIGS. 5J and 5T are Biacore profiles of ADI-11835.

FIGS. 6A-6D show SPR profiles of an Fab that comprises ADI-11815 binding to different domains of human CD33 and human CD33 having an R69G point mutation. FIG. 6A: Fab binding to human CD33 ECD; FIG. 6B: Fab binding to V domain; FIG. 6C: Fab binding to C domain: FIG. 6D: Fab binding to human CD33 having R69G.

FIGS. 7A-7D show SPR profiles of a Fab that comprises ADI-11801 binding to different domains of human CD33 and human CD33 having an R69G point mutation. FIG. 7A: human CD33 ECD; FIG. 7B: V domain; FIG. 7C: C domain: FIG. 7D: human CD33 having R69G.

FIG. 8 are bar graphs showing binding of monoclonal antibodies comprising CD33-binding clones to CD33 expressed on Molm-13 human AML cells. CD33 antibody Lintuzumab was also tested, and mean fluorescence intensity (MFI) was plotted. Five of the six antibodies show higher binding signal to CD33 compared to Lintuzumab.

FIG. 9 are bar graphs showing internalization of CD33 antibodies on Molm-13 cells after 24 hours. All the CD33 antibodies showed similar internalization after 24 hours. Lintuzumab showed slightly higher internalization compare to other anti-CD33 antibodies.

FIGS. 10A-10B show binding of CD33-targeting TriNKETs to human NKG2D expressed on EL4-hNKG2D and KHYG-1 cells. FIG. 10A shows binding of CD33-targeting TriNKETs to human NKG2D recombinantly expressed on EL4 cells. FIG. 10B shows binding of CD33-targeting TriNKETs to human NKG2D expressed on KHYG-1 cells. For each TriNKET, signal fold-over-background (FOB) was similar on both EL4-hNKG2D cells and KHYG-1 cells, and the rank of binding was also maintain on both cell lines.

FIG. 11 shows binding of CD33-targeting TriNKETs to CD33 expressed on human AML Molm-13 cells. Four different CD33-binding clones were used with five NKG2D-binding clones to make a total of 20 different TriNKETs. NKG2D-binding domains TriNKET do not affect the binding of CD33-binding clones to CD33.

FIG. 12 is a graph showing that rested human NK cells are activated by CD33-targeting TriNKETs in co-culture with CD33-expressing THP-1 AML cells.

FIG. 13 is a bar graph showing that CD33 TriNKETs induce rested NK cell mediated killing of Molm-13 AML cells.

FIG. 14 is a bar graph showing that CD33 TriNKETs induce activated NK cell mediated killing of THP-1 cells.

FIG. 15A are line graphs showing that TriNKETs mediate KHYG-1 killing of Molm-13 AML cells. FIG. 15B are line graphs showing that TriNKETs mediate rested human NK cell killing of Molm-13 human AML cells.

FIG. 16 are line graphs showing that TriNKETs mediate KHYG-1 killing of EOL-1 AML cells.

FIG. 17A are line graphs showing that TriNKETs mediate KHYG-1 killing of THP-1 cells. FIG. 17B are line graphs showing that TriNKETs mediate rested human NK cell killing of THP-1 human AML cells.

FIG. 18 is a representation of a multispecific binding protein that contains an NKG2D-binding domain (right arm), a CD33-binding domain (left arm), and an Fc domain or a portion thereof that binds to CD16.

FIG. 19 is a representation of a multispecific binding protein that includes a NKG2D-binding domain or a CD33-binding domain, either one of which can be in a scFv format, and an Fc domain or a portion thereof that binds to CD16.

FIG. 20 is a representation of a TriNKET in the Triomab form, which is a trifunctional, bispecific antibody that maintains an IgG-like shape. This chimera consists of two half antibodies, each with one light and one heavy chain, that originate from two parental antibodies.

FIG. 21 is a representation of a TriNKET in the KiH Common Light Chain (LC) form, which involves the knobs-into-holes (KIHs) technology. KiH is a heterodimer containing 2 Fabs binding to target 1 and 2, and an Fc stabilized by heterodimerization mutations. TriNKET in the KiH format may be an heterodimeric construct with 2 Fabs binding to target 1 and target 2, containing two different heavy chains and a common light chain that pairs with both heavy chains.

FIG. 22 is a representation of a TriNKET in the dual-variable domain immunoglobulin (DVD-Ig™) form, which combines the target binding domains of two monoclonal antibodies via flexible naturally occurring linkers, and yields a tetravalent IgG-like molecule. DVD-Ig™ is a homodimeric construct where variable domain targeting antigen 2 is fused to the N terminus of variable domain of Fab targeting antigen 1 Construct contains normal Fc.

FIG. 23 is a representation of a TriNKET in the Orthogonal Fab interface (Ortho-Fab) form, which is an heterodimeric construct that contains 2 Fabs binding to target1 and target 2 fused to Fc. LC-HC pairing is ensured by orthogonal interface. Heterodimerization is ensured by mutations in the Fc.

FIG. 24 is a representation of a TrinKET in the 2-in-1 Ig format.

FIG. 25 is a representation of a TriNKET in the ES form, which is an heterodimeric construct containing two different Fabs binding to target 1 and target 2 fused to the Fc. Heterodimerization is ensured by electrostatic steering mutations in the Fc.

FIG. 26 is a representation of a TriNKET in the Fab Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fabs binding to target 1 and 2, and an Fc stabilized by heterodimerization mutations.

FIG. 27 is a representation of a TriNKET in the SEED Body form, which is an heterodimer containing two Fabs binding to target 1 and 2, and an Fc stabilized by heterodimerization mutations.

FIG. 28 is a representation of a TriNKET in the LuZ-Y form, in which leucine zipper is used to induce heterodimerization of two different HCs. LuZ-Y form is a heterodimer containing two different scFabs binding to target 1 and 2, fused to Fc.

FIG. 29 is a representation of a TriNKET in the Cov-X-Body form.

FIGS. 30A-30B represent TriNKETs in the κλ-Body forms, which are an heterodimeric constructs with two different Fabs fused to Fc stabilized by heterodimerization mutations: Fab1 targeting antigen 1 contains kappa LC, while second Fab targeting antigen 2 contains lambda LC. FIG. 30A is an exemplary representation of one form of a κλ-Body; FIG. 30B is an exemplary representation of another κλ-Body.

FIG. 31 is an Oasc-Fab heterodimeric construct that includes Fab binding to target 1 and scFab binding to target 2 fused to Fc. Heterodimerization is ensured by mutations in the Fc.

FIG. 32 is a DuetMab, which is an heterodimeric construct containing two different Fabs binding to antigens 1 and 2, and Fc stabilized by heterodimerization mutations. Fab 1 and 2 contain differential S-S bridges that ensure correct light chain (LC) and heavy chain (HC) pairing.

FIG. 33 is a CrossmAb, which is an heterodimeric construct with two different Fabs binding to targets 1 and 2 fused to Fc stabilized by heterodimerization. CL and CH1 domains and VH and VL domains are switched, e.g., CH1 is fused in-line with VL, while CL is fused in-line with VH.

FIG. 34 is a Fit-Ig, which is a homodimeric constructs where Fab binding to antigen 2 is fused to the N terminus of HC of Fab that binds to antigen 1. The construct contains wild-type Fc.

FIG. 35 is a graph showing binding of A49-F3′-TriNKET-I07 and I07-F405L mAb to cell surface human NKG2D expressed on EL4 cells.

FIGS. 36A-36B are graphs showing binding of A49-F3′-TriNKET-I07 and 107-F405L mAb to CD33⁺ human AML cell lines Mv4-11 (FIG. 36A) and Molm-13 (FIG. 36B).

FIGS. 37A-37B are graphs showing internalization of A49-F3′-TriNKET-I07 and I07-F405L mAb after incubation with EOL-1 cells (FIG. 37A) and Molm-13 cells (FIG. 37B).

FIGS. 38A-38D are graphs showing specific lysis of Molm-13 (FIG. 38A), EOL-1 (FIG. 38B), and THP-1 (FIGS. 38C and 38D) human AML cells by rested human NK cells in the presence of A49-F3′-TriNKET-I07 and anti-CD33 monoclonal antibodies.

FIG. 39 is a series of flow cytograms showing the expression level of CD3, CD8, NKG2D, and CD16 on isolated primary CD8⁺ T cells.

FIGS. 40A-40B are graphs showing specific lysis of Molm-13 cells by isolated primary CD8⁺ T cells in the presence of A49-F3′-TriNKET-I07, A49-F3′-TriNKET-H76, a non-target TriNKET, or I07-F405L mAb (denoted as 107 in the figures). The primary CD8⁺ T cells in FIG. 40A were isolated from PBMCs of donor 1, and the primary CD8⁺ T cells in FIG. 40B were isolated from PBMCs of donor 2. The dotted lines indicate specific lysis of Molm-13 cells by CD8⁺ T cells in the absence of TriNKET or antibody.

FIGS. 41A-41E are histograms showing the binding of A49-F3′-TriNKET-I07 to NK cells (FIG. 41A), CD8⁺ T cells (FIG. 41B), CD4⁺ T cells (FIG. 41C), B cells (FIG. 41D), and monocytes (FIG. 41E) in human whole blood. The dotted lines without fill represent binding of A49-F3′-TriNKET-I07 to the cells; the solid lines with fill represent binding of human IgG1 isotype control to the cells.

FIGS. 42A-42B are graphs showing CD33 expression on monocytes. FIG. 42A shows CD33 expression on monocytes from four healthy donors (dark gray) and Molm-13 (light grey). The bottom five rows are signals from the cell samples stained with an anti-CD33 antibody; the top five rows are signals from the same samples stained with an isotype antibody. FIG. 42B shows CD33 expression on monocytes from the same donor before (light grey) and after (dark grey) negative selection for monocytes.

FIGS. 43A-43B are graphs showing long-term cytotoxicity of NK cells against Molm-13 AML cells and human primary monocytes in the presence of A49-F3′-TriNKET-I07. Proliferation of the target cells are plotted against the time of co-culture with NK cells in the presence of A49-F3′-TriNKET-I07 or PMA+ionomycin. FIG. 43A represents the results from an experiment using NK cells from one donor, and FIG. 43B represents the results from another experiment using NK cells from a different donor.

FIG. 44 illustrates a trispecific antibody (TriNKET) that contains a CD33-binding scFv, a NKG2D-targeting Fab, and a heterodimerized antibody constant domain that binds CD16. The antibody format is referred to herein as F3′-TriNKET.

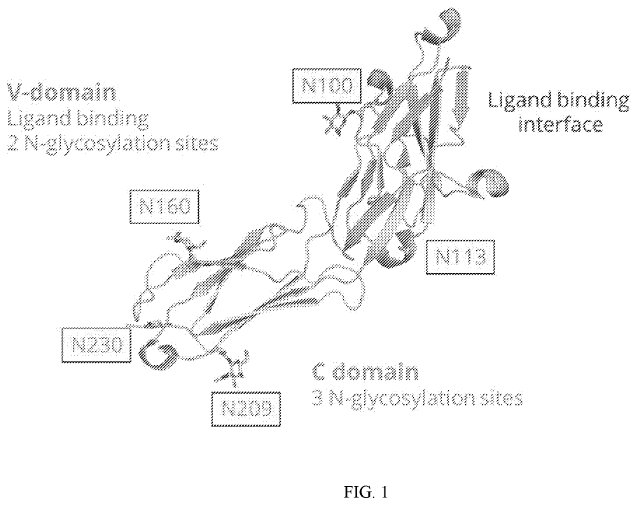

### DETAILED DESCRIPTION

In one aspect, the invention provides an antigen binding site that binds an epitope on an extracellular domain of human CD33 and/or Cynomolgus/Rhesus (cyno) CD33. In one aspect, the present invention provides an antigen binding site that recognizes and binds one or more conformational epitopes on the extracellular domain of human CD33 and/or Cynomolgus/Rhesus (cyno) CD33. In one aspect, the present invention provides an antigen binding site that recognizes and binds one or more conformational epitopes on the extracellular domain of human CD33 but does not recognize and/or bind one or more conformational epitopes on the extracellular domain of cyno CD33. In one aspect, the present invention provides an antigen binding site that binds to the R69G allele of human CD33. In one aspect, the present invention provides an antigen binding site that binds to wild-type human CD33 but not the R69G allele of human CD33. In one aspect, the present invention provides an antigen binding site that binds to an epitope on human CD33 that includes R69. In one aspect, the present invention provides an antigen binding site that binds to the S128N allele of human CD33. In one aspect, the present invention provides an antigen binding site that binds to wild-type human CD33 but not the S128N allele of human CD33. In one aspect, the present invention provides an antigen binding site that binds to an epitope on human CD33 that includes S128. In one aspect, the present invention provides an antigen binding site including a heavy chain variable domain, which binds to the extracellular domain in human CD33 and/or cyno CD33, irrespective of the glycosylation profile of the targeted CD33.

In certain embodiments the present invention provides an antigen binding site, which binds to the extracellular domain in human CD33 and/or cyno CD33, such that the epitopes are unique compared to the epitopes targeted by one or more known anti-CD33 antibodies in the art. In certain embodiments, the present invention provides an antigen binding site, which binds to the extracellular domain in human CD33 and/or cyno CD33, and shows human or Cynomolgus/Rhesus (cyno) CD33 cross reactivity and high affinity binding to the target CD33.

The invention provides antigen-binding proteins that bind CD33 on a cancer cell and pharmaceutical compositions comprising such proteins, and therapeutic methods using such proteins and pharmaceutical compositions, including for the treatment of cancer. Various aspects of the invention are set forth in the sections below; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the invention are discussed in more detail below.

### I. Antigen-Binding Site

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:1, and an antibody light chain variable domain that includes an amino acid sequence at least 90% (e.g., at least 91%, 92%, 93%, 94%, 95%, 96%, 97%, 98%, 99%, or 100%) identical to SEQ ID NO:2. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:1, includes a CDR1 sequence represented by the amino acid sequence of SEQ ID NO:21, a CDR2 sequence represented by the amino acid sequence of SEQ ID NO:22, and a CDR3 sequence represented by the amino acid sequence of SEQ ID NO:23. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:1, includes a CDR1 sequence represented by the amino acid sequence of SEQ ID NO:434, a CDR2 sequence represented by the amino acid sequence of SEQ ID NO:22, and a CDR3 sequence represented by the amino acid sequence of SEQ ID NO:435. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:2, includes a CDR1 sequence represented by the amino acid sequence of SEQ ID NO:24, a CDR2 sequence represented by the amino acid sequence of SEQ ID NO:25, and a CDR3 sequence represented by the amino acid sequence of SEQ ID NO:26.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:3, and an antibody light chain variable domain that includes an amino acid sequence at least 90% (e.g., at least 91%, 92%, 93%, 94%, 95%, 96%, 97%, 98%, 99%, or 100%) identical to SEQ ID NO:4. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:3, includes a CDR1 sequence represented by the amino acid sequence of SEQ ID NO:27, a CDR2 sequence represented by the amino acid sequence of SEQ ID NO:28, and a CDR3 sequence represented by the amino acid sequence of SEQ ID NO:29. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:3, includes a CDR1 sequence represented by the amino acid sequence of SEQ ID NO:181, a CDR2 sequence represented by the amino acid sequence of SEQ ID NO:28, and a CDR3 sequence represented by the amino acid sequence of SEQ ID NO:436. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:4, includes a CDR1 sequence represented by the amino acid sequence of SEQ ID NO:30, a CDR2 sequence represented by the amino acid sequence of SEQ ID NO:31, and a CDR3 sequence represented by the amino acid sequence of SEQ ID NO:32.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., at least 91%, 92%, 93%, 94%, 95%, 96%, 97%, 98%, 99%, or 100%) identical to SEQ ID NO:6. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:5, includes a CDR1 sequence represented by the amino acid sequence of SEQ ID NO:33, a CDR2 sequence represented by the amino acid sequence of SEQ ID NO:34, and a CDR3 sequence represented by the amino acid sequence of SEQ ID NO:35. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:5, includes a CDR1 sequence represented by the amino acid sequence of SEQ ID NO:183, a CDR2 sequence represented by the amino acid sequence of SEQ ID NO:34, and a CDR3 sequence represented by the amino acid sequence of SEQ ID NO:184. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:6, includes a CDR1 sequence represented by the amino acid sequence of SEQ ID NO:36, a CDR2 sequence represented by the amino acid sequence of SEQ ID NO:37, and a CDR3 sequence represented by the amino acid sequence of SEQ ID NO:38. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:6, includes a CDR1 sequence represented by the amino acid sequence of SEQ ID NO:36, a CDR2 sequence represented by the amino acid sequence of SEQ ID NO:185, and a CDR3 sequence represented by the amino acid sequence of SEQ ID NO:38. In certain embodiments, the antigen-binding site comprises an amino acid sequence at least 90% (e.g., at least 90%, 91%, 92%, 93%, 94%, 95%, 96%, 97%, 98%, 99%, or 100%) identical to SEQ ID NO:188. In certain embodiments, the antigen-binding site comprises an amino acid sequence at least 95% identical to SEQ ID NO:188. In certain embodiments, the antigen-binding site comprises an amino acid sequence at least 99% identical to SEQ ID NO:188. In certain embodiments, the antigen-binding site comprises the amino acid sequence of SEQ ID NO:188.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:7, and an antibody light chain variable domain that includes an amino acid sequence at least 90% (e.g., at least 91%, 92%, 93%, 94%, 95%, 96%, 97%, 98%, 99%, or 100%) identical to SEQ ID NO:8. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:7, includes a CDR1 sequence represented by the amino acid sequence of SEQ ID NO:39, a CDR2 sequence represented by the amino acid sequence of SEQ ID NO:40, and a CDR3 sequence represented by the amino acid sequence of SEQ ID NO:41. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:7, includes a CDR1 sequence represented by the amino acid sequence of SEQ ID NO:437, a CDR2 sequence represented by the amino acid sequence of SEQ ID NO:40, and a CDR3 sequence represented by the amino acid sequence of SEQ ID NO:438. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:42, a CDR2 sequence represented by the amino acid sequence of SEQ ID NO:43, and a CDR3 sequence represented by the amino acid sequence of SEQ ID NO:44.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:9, and an antibody light chain variable domain that includes an amino acid sequence at least 90% (e.g., at least 91%, 92%, 93%, 94%, 95%, 96%, 97%, 98%, 99%, or 100%) identical to SEQ ID NO:10. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:9, includes a CDR1 sequence represented by the amino acid sequence of SEQ ID NO:45, a CDR2 sequence represented by the amino acid sequence of SEQ ID NO:46, and a CDR3 sequence represented by the amino acid sequence of SEQ ID NO:47. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:9, includes a CDR1 sequence represented by the amino acid sequence of SEQ ID NO:181, a CDR2 sequence represented by the amino acid sequence of SEQ ID NO:46, and a CDR3 sequence represented by the amino acid sequence of SEQ ID NO:182. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:10, includes a CDR1 sequence represented by the amino acid sequence of SEQ ID NO:48, a CDR2 sequence represented by the amino acid sequence of SEQ ID NO:49, and a CDR3 sequence represented by the amino acid sequence of SEQ ID NO:50. In certain embodiments, the antigen-binding site comprises an amino acid sequence at least 90% (e.g., at least 91%, 92%, 93%, 94%, 95%, 96%, 97%, 98%, 99%, or 100%) identical to SEQ ID NO:198. In certain embodiments, the antigen-binding site comprises an amino acid sequence at least 95% identical to SEQ ID NO:198. In certain embodiments, the antigen-binding site comprises an amino acid sequence at least 99% identical to SEQ ID NO:198. In certain embodiments, the antigen-binding site comprises the amino acid sequence of SEQ ID NO:198.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:11, and an antibody light chain variable domain that includes an amino acid sequence at least 90% (e.g., at least 91%, 92%, 93%, 94%, 95%, 96%, 97%, 98%, 99%, or 100%) identical to SEQ ID NO:12. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:11, includes a CDR1 sequence represented by the amino acid sequence of SEQ ID NO:51, a CDR2 sequence represented by the amino acid sequence of SEQ ID NO:52, and a CDR3 sequence represented by the amino acid sequence of SEQ ID NO:53. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:11, includes a CDR1 sequence represented by the amino acid sequence of SEQ ID NO:181, a CDR2 sequence represented by the amino acid sequence of SEQ ID NO:52, and a CDR3 sequence represented by the amino acid sequence of SEQ ID NO:439. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:12, includes a CDR1 sequence represented by the amino acid sequence of SEQ ID NO:54, a CDR2 sequence represented by the amino acid sequence of SEQ ID NO:55, and a CDR3 sequence represented by the amino acid sequence of SEQ ID NO:56.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:13, and an antibody light chain variable domain that includes an amino acid sequence at least 90% (e.g., at least 91%, 92%, 93%, 94%, 95%, 96%, 97%, 98%, 99%, or 100%) identical to SEQ ID NO:14. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:13, includes a CDR1 sequence represented by the amino acid sequence of SEQ ID NO:57, a CDR2 sequence represented by the amino acid sequence of SEQ ID NO:58, and a CDR3 sequence represented by the amino acid sequence of SEQ ID NO:59. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:13, includes a CDR1 sequence represented by the amino acid sequence of SEQ ID NO:440, a CDR2 sequence represented by the amino acid sequence of SEQ ID NO:58, and a CDR3 sequence represented by the amino acid sequence of SEQ ID NO:441. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:14, includes a CDR1 sequence represented by the amino acid sequence of SEQ ID NO:60, a CDR2 sequence represented by the amino acid sequence of SEQ ID NO:61, and a CDR3 sequence represented by the amino acid sequence of SEQ ID NO:62.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:15, and an antibody light chain variable domain that includes an amino acid sequence at least 90% (e.g., at least 91%, 92%, 93%, 94%, 95%, 96%, 97%, 98%, 99%, or 100%) identical to SEQ ID NO:16. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:15, includes a CDR1 sequence represented by the amino acid sequence of SEQ ID NO:63, a CDR2 sequence represented by the amino acid sequence of SEQ ID NO:64, and a CDR3 sequence represented by the amino acid sequence of SEQ ID NO:65. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:15, includes a CDR1 sequence represented by the amino acid sequence of SEQ ID NO:442, a CDR2 sequence represented by the amino acid sequence of SEQ ID NO:64, and a CDR3 sequence represented by the amino acid sequence of SEQ ID NO:443. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:16, includes a CDR1 sequence represented by the amino acid sequence of SEQ ID NO:66, a CDR2 sequence represented by the amino acid sequence of SEQ ID NO:67, and a CDR3 sequence represented by the amino acid sequence of SEQ ID NO:68.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:17, and an antibody light chain variable domain that includes an amino acid sequence at least 90% (e.g., at least 91%, 92%, 93%, 94%, 95%, 96%, 97%, 98%, 99%, or 100%) identical to SEQ ID NO:18. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:17, includes a CDR1 sequence represented by the amino acid sequence of SEQ ID NO:69, a CDR2 sequence represented by the amino acid sequence of SEQ ID NO:70, and a CDR3 sequence represented by the amino acid sequence of SEQ ID NO:71. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:17, includes a CDR1 sequence represented by the amino acid sequence of SEQ ID NO:181, a CDR2 sequence represented by the amino acid sequence of SEQ ID NO:70, and a CDR3 sequence represented by the amino acid sequence of SEQ ID NO:444. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:18, includes a CDR1 sequence represented by the amino acid sequence of SEQ ID NO:72, a CDR2 sequence represented by the amino acid sequence of SEQ ID NO:73, and a CDR3 sequence represented by the amino acid sequence of SEQ ID NO:74.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:19, and an antibody light chain variable domain that includes an amino acid sequence at least 90% (e.g., at least 91%, 92%, 93%, 94%, 95%, 96%, 97%, 98%, 99%, or 100%) identical to SEQ ID NO:20. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:19, includes a CDR1 sequence represented by the amino acid sequence of SEQ ID NO:75, a CDR2 sequence represented by the amino acid sequence of SEQ ID NO:76, and a CDR3 sequence represented by the amino acid sequence of SEQ ID NO:77. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:19, includes a CDR1 sequence represented by the amino acid sequence of SEQ ID NO:445, a CDR2 sequence represented by the amino acid sequence of SEQ ID NO:76, and a CDR3 sequence represented by the amino acid sequence of SEQ ID NO:446. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:20, includes a CDR1 sequence represented by the amino acid sequence of SEQ ID NO:78, a CDR2 sequence represented by the amino acid sequence of SEQ ID NO:79, and a CDR3 sequence represented by the amino acid sequence of SEQ ID NO:80.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:266, and an antibody light chain variable domain that includes an amino acid sequence at least 90% (e.g., at least 91%, 92%, 93%, 94%, 95%, 96%, 97%, 98%, 99%, or 100%) identical to SEQ ID NO:267. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:266, includes a CDR1 sequence represented by the amino acid sequence of SEQ ID NO:304, a CDR2 sequence represented by the amino acid sequence of SEQ ID NO:305, and a CDR3 sequence represented by the amino acid sequence of SEQ ID NO:306. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:266, includes a CDR1 sequence represented by the amino acid sequence of SEQ ID NO:528, a CDR2 sequence represented by the amino acid sequence of SEQ ID NO:305, and a CDR3 sequence represented by the amino acid sequence of SEQ ID NO:529. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:267, includes a CDR1 sequence represented by the amino acid sequence of SEQ ID NO:307, a CDR2 sequence represented by the amino acid sequence of SEQ ID NO:308, and a CDR3 sequence represented by the amino acid sequence of SEQ ID NO:309.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:268, and an antibody light chain variable domain that includes an amino acid sequence at least 90% (e.g., at least 91%, 92%, 93%, 94%, 95%, 96%, 97%, 98%, 99%, or 100%) identical to SEQ ID NO:269. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:268, includes a CDR1 sequence represented by the amino acid sequence of SEQ ID NO:310, a CDR2 sequence represented by the amino acid sequence of SEQ ID NO:311, and a CDR3 sequence represented by the amino acid sequence of SEQ ID NO:312. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:268, includes a CDR1 sequence represented by the amino acid sequence of SEQ ID NO:530, a CDR2 sequence represented by the amino acid sequence of SEQ ID NO:311, and a CDR3 sequence represented by the amino acid sequence of SEQ ID NO:531. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:269, includes a CDR1 sequence represented by the amino acid sequence of SEQ ID NO:313, a CDR2 sequence represented by the amino acid sequence of SEQ ID NO:314, and a CDR3 sequence represented by the amino acid sequence of SEQ ID NO:315.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:270, and an antibody light chain variable domain that includes an amino acid sequence at least 90% (e.g., at least 91%, 92%, 93%, 94%, 95%, 96%, 97%, 98%, 99%, or 100%) identical to SEQ ID NO:271. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:270, includes a CDR1 sequence represented by the amino acid sequence of SEQ ID NO:316, a CDR2 sequence represented by the amino acid sequence of SEQ ID NO:317, and a CDR3 sequence represented by the amino acid sequence of SEQ ID NO:318. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:270, includes a CDR1 sequence represented by the amino acid sequence of SEQ ID NO:532, a CDR2 sequence represented by the amino acid sequence of SEQ ID NO:317, and a CDR3 sequence represented by the amino acid sequence of SEQ ID NO:533. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:271, includes a CDR1 sequence represented by the amino acid sequence of SEQ ID NO:319, a CDR2 sequence represented by the amino acid sequence of SEQ ID NO:320, and a CDR3 sequence represented by the amino acid sequence of SEQ ID NO:321.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:272, and an antibody light chain variable domain that includes an amino acid sequence at least 90% (e.g., at least 91%, 92%, 93%, 94%, 95%, 96%, 97%, 98%, 99%, or 100%) identical to SEQ ID NO:273. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:272, includes a CDR1 sequence represented by the amino acid sequence of SEQ ID NO:322, a CDR2 sequence represented by the amino acid sequence of SEQ ID NO:323, and a CDR3 sequence represented by the amino acid sequence of SEQ ID NO:324. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:272, includes a CDR1 sequence represented by the amino acid sequence of SEQ ID NO:534, a CDR2 sequence represented by the amino acid sequence of SEQ ID NO:323, and a CDR3 sequence represented by the amino acid sequence of SEQ ID NO:535. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:273, includes a CDR1 sequence represented by the amino acid sequence of SEQ ID NO:325, a CDR2 sequence represented by the amino acid sequence of SEQ ID NO:326, and a CDR3 sequence represented by the amino acid sequence of SEQ ID NO:327.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:274, and an antibody light chain variable domain that includes an amino acid sequence at least 90% (e.g., at least 91%, 92%, 93%, 94%, 95%, 96%, 97%, 98%, 99%, or 100%) identical to SEQ ID NO:275. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:274, includes a CDR1 sequence represented by the amino acid sequence of SEQ ID NO:328, a CDR2 sequence represented by the amino acid sequence of SEQ ID NO:329, and a CDR3 sequence represented by the amino acid sequence of SEQ ID NO:330. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:274, includes a CDR1 sequence represented by the amino acid sequence of SEQ ID NO:536, a CDR2 sequence represented by the amino acid sequence of SEQ ID NO:329, and a CDR3 sequence represented by the amino acid sequence of SEQ ID NO:537. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:275, includes a CDR1 sequence represented by the amino acid sequence of SEQ ID NO:331, a CDR2 sequence represented by the amino acid sequence of SEQ ID NO:332, and a CDR3 sequence represented by the amino acid sequence of SEQ ID NO:333.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:276, and an antibody light chain variable domain that includes an amino acid sequence at least 90% (e.g., at least 91%, 92%, 93%, 94%, 95%, 96%, 97%, 98%, 99%, or 100%) identical to SEQ ID NO:277. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:276, includes a CDR1 sequence represented by the amino acid sequence of SEQ ID NO:334, a CDR2 sequence represented by the amino acid sequence of SEQ ID NO:335, and a CDR3 sequence represented by the amino acid sequence of SEQ ID NO:336. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:276, includes a CDR1 sequence represented by the amino acid sequence of SEQ ID NO:538, a CDR2 sequence represented by the amino acid sequence of SEQ ID NO:335, and a CDR3 sequence represented by the amino acid sequence of SEQ ID NO:539. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:277, includes a CDR1 sequence represented by the amino acid sequence of SEQ ID NO:337, a CDR2 sequence represented by the amino acid sequence of SEQ ID NO:338, and a CDR3 sequence represented by the amino acid sequence of SEQ ID NO:339.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:278, and an antibody light chain variable domain that includes an amino acid sequence at least 90% (e.g., at least 91%, 92%, 93%, 94%, 95%, 96%, 97%, 98%, 99%, or 100%) identical to SEQ ID NO:279. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:278, includes a CDR1 sequence represented by the amino acid sequence of SEQ ID NO:340, a CDR2 sequence represented by the amino acid sequence of SEQ ID NO:341, and a CDR3 sequence represented by the amino acid sequence of SEQ ID NO:342. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:278, includes a CDR1 sequence represented by the amino acid sequence of SEQ ID NO:540, a CDR2 sequence represented by the amino acid sequence of SEQ ID NO:341, and a CDR3 sequence represented by the amino acid sequence of SEQ ID NO:541. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:279, includes a CDR1 sequence represented by the amino acid sequence of SEQ ID NO:343, a CDR2 sequence represented by the amino acid sequence of SEQ ID NO:344, and a CDR3 sequence represented by the amino acid sequence of SEQ ID NO:345.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:280, and an antibody light chain variable domain that includes an amino acid sequence at least 90% (e.g., at least 91%, 92%, 93%, 94%, 95%, 96%, 97%, 98%, 99%, or 100%) identical to SEQ ID NO:281. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:280, includes a CDR1 sequence represented by the amino acid sequence of SEQ ID NO:346, a CDR2 sequence represented by the amino acid sequence of SEQ ID NO:347, and a CDR3 sequence represented by the amino acid sequence of SEQ ID NO:348. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:280, includes a CDR1 sequence represented by the amino acid sequence of SEQ ID NO:542, a CDR2 sequence represented by the amino acid sequence of SEQ ID NO:347, and a CDR3 sequence represented by the amino acid sequence of SEQ ID NO:543. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:281, includes a CDR1 sequence represented by the amino acid sequence of SEQ ID NO:349, a CDR2 sequence represented by the amino acid sequence of SEQ ID NO:350, and a CDR3 sequence represented by the amino acid sequence of SEQ ID NO:351.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:282, and an antibody light chain variable domain that includes an amino acid sequence at least 90% (e.g., at least 91%, 92%, 93%, 94%, 95%, 96%, 97%, 98%, 99%, or 100%) identical to SEQ ID NO:283. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:282, includes a CDR1 sequence represented by the amino acid sequence of SEQ ID NO:352, a CDR2 sequence represented by the amino acid sequence of SEQ ID NO:353, and a CDR3 sequence represented by the amino acid sequence of SEQ ID NO:354. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:282, includes a CDR1 sequence represented by the amino acid sequence of SEQ ID NO:544, a CDR2 sequence represented by the amino acid sequence of SEQ ID NO:353, and a CDR3 sequence represented by the amino acid sequence of SEQ ID NO:545. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:283, includes a CDR1 sequence represented by the amino acid sequence of SEQ ID NO:355, a CDR2 sequence represented by the amino acid sequence of SEQ ID NO:356, and a CDR3 sequence represented by the amino acid sequence of SEQ ID NO:357.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:284, and an antibody light chain variable domain that includes an amino acid sequence at least 90% (e.g., at least 91%, 92%, 93%, 94%, 95%, 96%, 97%, 98%, 99%, or 100%) identical to SEQ ID NO:285. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:284, includes a CDR1 sequence represented by the amino acid sequence of SEQ ID NO:358, a CDR2 sequence represented by the amino acid sequence of SEQ ID NO:359, and a CDR3 sequence represented by the amino acid sequence of SEQ ID NO:360. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:284, includes a CDR1 sequence represented by the amino acid sequence of SEQ ID NO:546, a CDR2 sequence represented by the amino acid sequence of SEQ ID NO:359, and a CDR3 sequence represented by the amino acid sequence of SEQ ID NO:547. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:285, includes a CDR1 sequence represented by the amino acid sequence of SEQ ID NO:361, a CDR2 sequence represented by the amino acid sequence of SEQ ID NO:362, and a CDR3 sequence represented by the amino acid sequence of SEQ ID NO:363.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:286, and an antibody light chain variable domain that includes an amino acid sequence at least 90% (e.g., at least 91%, 92%, 93%, 94%, 95%, 96%, 97%, 98%, 99%, or 100%) identical to SEQ ID NO:287. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:286, includes a CDR1 sequence represented by the amino acid sequence of SEQ ID NO:364, a CDR2 sequence represented by the amino acid sequence of SEQ ID NO:365, and a CDR3 sequence represented by the amino acid sequence of SEQ ID NO:366. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:286, includes a CDR1 sequence represented by the amino acid sequence of SEQ ID NO:548, a CDR2 sequence represented by the amino acid sequence of SEQ ID NO:365, and a CDR3 sequence represented by the amino acid sequence of SEQ ID NO:549. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:287, includes a CDR1 sequence represented by the amino acid sequence of SEQ ID NO:367, a CDR2 sequence represented by the amino acid sequence of SEQ ID NO:368, and a CDR3 sequence represented by the amino acid sequence of SEQ ID NO:369.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:288, and an antibody light chain variable domain that includes an amino acid sequence at least 90% (e.g., at least 91%, 92%, 93%, 94%, 95%, 96%, 97%, 98%, 99%, or 100%) identical to SEQ ID NO:289. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:288, includes a CDR1 sequence represented by the amino acid sequence of SEQ ID NO:370, a CDR2 sequence represented by the amino acid sequence of SEQ ID NO:371, and a CDR3 sequence represented by the amino acid sequence of SEQ ID NO:372. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:288, includes a CDR1 sequence represented by the amino acid sequence of SEQ ID NO:550, a CDR2 sequence represented by the amino acid sequence of SEQ ID NO:371, and a CDR3 sequence represented by the amino acid sequence of SEQ ID NO:551. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:289, includes a CDR1 sequence represented by the amino acid sequence of SEQ ID NO:373, a CDR2 sequence represented by the amino acid sequence of SEQ ID NO:374, and a CDR3 sequence represented by the amino acid sequence of SEQ ID NO:375.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:290, and an antibody light chain variable domain that includes an amino acid sequence at least 90% (e.g., at least 91%, 92%, 93%, 94%, 95%, 96%, 97%, 98%, 99%, or 100%) identical to SEQ ID NO:291. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:290, includes a CDR1 sequence represented by the amino acid sequence of SEQ ID NO:376, a CDR2 sequence represented by the amino acid sequence of SEQ ID NO:377, and a CDR3 sequence represented by the amino acid sequence of SEQ ID NO:378. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:290, includes a CDR1 sequence represented by the amino acid sequence of SEQ ID NO:552, a CDR2 sequence represented by the amino acid sequence of SEQ ID NO:377, and a CDR3 sequence represented by the amino acid sequence of SEQ ID NO:553. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:291, includes a CDR1 sequence represented by the amino acid sequence of SEQ ID NO:379, a CDR2 sequence represented by the amino acid sequence of SEQ ID NO:380, and a CDR3 sequence represented by the amino acid sequence of SEQ ID NO:381.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:292, and an antibody light chain variable domain that includes an amino acid sequence at least 90% (e.g., at least 91%, 92%, 93%, 94%, 95%, 96%, 97%, 98%, 99%, or 100%) identical to SEQ ID NO:293. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:292, includes a CDR1 sequence represented by the amino acid sequence of SEQ ID NO:382, a CDR2 sequence represented by the amino acid sequence of SEQ ID NO:383, and a CDR3 sequence represented by the amino acid sequence of SEQ ID NO:384. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:292, includes a CDR1 sequence represented by the amino acid sequence of SEQ ID NO:554, a CDR2 sequence represented by the amino acid sequence of SEQ ID NO:383, and a CDR3 sequence represented by the amino acid sequence of SEQ ID NO:555. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:293, includes a CDR1 sequence represented by the amino acid sequence of SEQ ID NO:385, a CDR2 sequence represented by the amino acid sequence of SEQ ID NO:386, and a CDR3 sequence represented by the amino acid sequence of SEQ ID NO:387.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:294, and an antibody light chain variable domain that includes an amino acid sequence at least 90% (e.g., at least 91%, 92%, 93%, 94%, 95%, 96%, 97%, 98%, 99%, or 100%) identical to SEQ ID NO:295. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:294, includes a CDR1 sequence represented by the amino acid sequence of SEQ ID NO:388, a CDR2 sequence represented by the amino acid sequence of SEQ ID NO:389, and a CDR3 sequence represented by the amino acid sequence of SEQ ID NO:390. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:294, includes a CDR1 sequence represented by the amino acid sequence of SEQ ID NO:556, a CDR2 sequence represented by the amino acid sequence of SEQ ID NO:389, and a CDR3 sequence represented by the amino acid sequence of SEQ ID NO:557. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:295, includes a CDR1 sequence represented by the amino acid sequence of SEQ ID NO:391, a CDR2 sequence represented by the amino acid sequence of SEQ ID NO:392, and a CDR3 sequence represented by the amino acid sequence of SEQ ID NO:393.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:296, and an antibody light chain variable domain that includes an amino acid sequence at least 90% (e.g., at least 91%, 92%, 93%, 94%, 95%, 96%, 97%, 98%, 99%, or 100%) identical to SEQ ID NO:297. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:296, includes a CDR1 sequence represented by the amino acid sequence of SEQ ID NO:394, a CDR2 sequence represented by the amino acid sequence of SEQ ID NO:395, and a CDR3 sequence represented by the amino acid sequence of SEQ ID NO:396. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:296, includes a CDR1 sequence represented by the amino acid sequence of SEQ ID NO:558, a CDR2 sequence represented by the amino acid sequence of SEQ ID NO:395, and a CDR3 sequence represented by the amino acid sequence of SEQ ID NO:559. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:297, includes a CDR1 sequence represented by the amino acid sequence of SEQ ID NO:397, a CDR2 sequence represented by the amino acid sequence of SEQ ID NO:398, and a CDR3 sequence represented by the amino acid sequence of SEQ ID NO:399.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:298, and an antibody light chain variable domain that includes an amino acid sequence at least 90% (e.g., at least 91%, 92%, 93%, 94%, 95%, 96%, 97%, 98%, 99%, or 100%) identical to SEQ ID NO:299. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:298, includes a CDR1 sequence represented by the amino acid sequence of SEQ ID NO:400, a CDR2 sequence represented by the amino acid sequence of SEQ ID NO:401, and a CDR3 sequence represented by the amino acid sequence of SEQ ID NO:402. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:298, includes a CDR1 sequence represented by the amino acid sequence of SEQ ID NO:560, a CDR2 sequence represented by the amino acid sequence of SEQ ID NO:401, and a CDR3 sequence represented by the amino acid sequence of SEQ ID NO:561. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:299, includes a CDR1 sequence represented by the amino acid sequence of SEQ ID NO:403, a CDR2 sequence represented by the amino acid sequence of SEQ ID NO:404, and a CDR3 sequence represented by the amino acid sequence of SEQ ID NO:405.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:300, and an antibody light chain variable domain that includes an amino acid sequence at least 90% (e.g., at least 91%, 92%, 93%, 94%, 95%, 96%, 97%, 98%, 99%, or 100%) identical to SEQ ID NO:301. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:300, includes a CDR1 sequence represented by the amino acid sequence of SEQ ID NO:406, a CDR2 sequence represented by the amino acid sequence of SEQ ID NO:407, and a CDR3 sequence represented by the amino acid sequence of SEQ ID NO:408. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:300, includes a CDR1 sequence represented by the amino acid sequence of SEQ ID NO:562, a CDR2 sequence represented by the amino acid sequence of SEQ ID NO:407, and a CDR3 sequence represented by the amino acid sequence of SEQ ID NO:563. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:301, includes a CDR1 sequence represented by the amino acid sequence of SEQ ID NO:409, a CDR2 sequence represented by the amino acid sequence of SEQ ID NO:410, and a CDR3 sequence represented by the amino acid sequence of SEQ ID NO:411.

In certain embodiments, the present invention provides an antigen-binding site that includes an antibody heavy chain variable domain that includes an amino acid sequence at least 90% (e.g., at least 91%, 92%, 93%, 94%, 95%, 96%, 97%, 98%, 99%, or 100%) identical to the amino acid sequence of SEQ ID NO:302, and an antibody light chain variable domain that includes an amino acid sequence at least 90% (e.g., at least 91%, 92%, 93%, 94%, 95%, 96%, 97%, 98%, 99%, or 100%) identical to SEQ ID NO:303. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:302, includes a CDR1 sequence represented by the amino acid sequence of SEQ ID NO:412, a CDR2 sequence represented by the amino acid sequence of SEQ ID NO:413, and a CDR3 sequence represented by the amino acid sequence of SEQ ID NO:414. In certain embodiments, an antigen-binding site that includes an antibody heavy chain variable domain with an amino acid sequence at least 90% (e.g., at least 91%, 92%, 93%, 94%, 95%, 96%, 97%, 98%, 99%, or 100%) identical to the amino acid sequence of SEQ ID NO:302, includes a CDR1 sequence represented by the amino acid sequence of SEQ ID NO:564, a CDR2 sequence represented by the amino acid sequence of SEQ ID NO:413, and a CDR3 sequence represented by the amino acid sequence of SEQ ID NO:565. In certain embodiments, an antigen-binding site that includes an antibody light chain variable domain with an amino acid sequence at least 90% (e.g., at least 91%, 92%, 93%, 94%, 95%, 96%, 97%, 98%, 99%, or 100%) identical to the amino acid sequence of SEQ ID NO:303, includes a CDR1 sequence represented by the amino acid sequence of SEQ ID NO:415, a CDR2 sequence represented by the amino acid sequence of SEQ ID NO:416, and a CDR3 sequence represented by the amino acid sequence of SEQ ID NO:417.

In each of the foregoing embodiments, it is contemplated herein that immunoglobulin heavy chain variable region sequences and/or light chain variable region sequences that together bind CD33 may contain amino acid alterations (e.g., at least 1, 2, 3, 4, 5, or 10 amino acid substitutions, deletions, or additions) in the framework regions of the heavy and/or light chain variable regions without affecting their ability to bind to CD33 significantly.

Table 1 lists peptide sequences of heavy chain variable domains and light chain variable domains that, in combination (either as a Fab fragment or a single-chain variable fragment (scFv)), can bind to CD33. Unless indicated otherwise, the CDR sequences provided in Table 1 are determined under Kabat. The CD33-binding domains can vary in their binding affinity to CD33. Table 1 also lists scFv forms of the CD33-binding heavy and light chain variable domains. The exemplary nucleic acid sequences listed in Table 1 are predicted possible nucleic acid sequences that the listed corresponding peptide sequences originated from, and were generated using EMBL-EBI's Protein Sequence Back-translation program.

TABLE 1

| | VH | VL |
|---|---|---|
| ADI-10159 | EVQLVESGGGLVQPGGSLRLSC | DIQMTQSPSTLSASVGDRVTITCR |
| [Ab1] | AASGFTFSSYGMSWVRQAPGK | ASQSISSWLAWYQQKPGKAPKL |
| (G59) | GLEWVANIKQDGSEKYYVDSV | LIYDASSLESGVPSRFSGSGSGTE |
| | KGRFTISRDNAKNSLYLQMNSL | FTLTISSLQPDDFATYYCQQYESF |
| | RAEDTAVYYCAREGGPYYDSS | PTFGGGTKVEIK [SEQ ID NO: 2] |
| | GYFVYYGMDVWGQGTTVTVSS | CDR1: RASQSISSWLA [SEQ ID |
| | [SEQ ID NO: 1] | NO: 24] |
| | CDR1: FTFSSYGMS [SEQ ID | CDR2: DASSLES [SEQ ID NO: 25] |
| | NO: 21](non-Kabat) or SYGMS | CDR3: QQYESFPT [SEQ ID NO: 26] |
| | [SEQ ID NO: 434] | |
| | CDR2: NIKQDGSEKYYVDSVKG | |
| | [SEQ ID NO: 22] | |
| | CDR3: | |
| | AREGGPYYDSSGYFVYYGMDV | |
| | [SEQ ID NO: 23](non-Kabat) or | |
| | EGGPYYDSSGYFVYYGMDV | |
| | [SEQ ID NO: 435] | |
| scFv of | DIQMTQSPSTLSASVGDRVTITCRASQSISSWLAWYQQKPGKAPKLLI | |
| Ab1 | YDASSLESGVPSRFSGSGSGTEFTLTISSLQPDDFATYYCQQYESFPTF | |
| | G [symbol] GTKVEIKGGGGSGGGGSGGGGSGGGGSEVQLVESGGGLVQPG | |
| | GSLRLSCAASGFTFSSYGMSWVRQAPGK [symbol] LEWVANIKQDGSEKYYV | |
| | DSVKGRFTISRDNAKNSLYLQMNSLRAEDTAVYYCAREGGPYYDSS | |
| | GYFVYYGMDVWGQGTTVTVSS [SEQ ID NO: 206] | |
| | EVQLVESGGGLVQPGGSLRLSCAASGFTFSSYGMSWVRQAPGK [symbol] LE | |
| | WVANIKQDGSEKYYVDSVKGRFTISRDNAKNSLYLQMNSLRAEDTA | |
| | VYYCAREGGPYYDSSGYFVYYGMDVWGQGTTVTVSSGGGGSGGG | |
| | GSGGGGSGGGGSDIQMTQSPSTLSASVGDRVTITCRASQSISSWLA | |
| | WYQQKPGKAPKLLIYDASSLESGVPSRFSGSGSGTEFTLTISSLQPDD | |
| | FATYYCQQYESFPTFG [symbol] GTKVEIK [SEQ ID NO: 207] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCCAGAGCATCA | |
| sequence | GCAGCTGGCTGGCCTGGTACCAGCAGAAGCCCGGCAAGGCCCCC | |
| of Ab1 | AAGCTGCTGATCTACGACGCCAGCAGCCTGGAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGTACGAGAGCTTCCCCACCTTCGGCTGCGGCACCAAGGTGG | |
| | AGATCAAGGGCGGCGGCGGCAGCGGCGGCGGCGGCAGCGGCGGC | |
| | GGCGGCAGCGGCGGCGGCGGCAGCGAGGTGCAGCTGGTGGAGAG | |
| | CGGCGGCGGCCTGGTGCAGCCCGGCGGCAGCCTGAGGCTGAGCT | |
| | GCGCCGCCAGCGGCTTCACCTTCAGCAGCTACGGCATGAGCTGGG | |
| | TGAGGCAGGCCCCCGGCAAGTGCCTGGAGTGGGTGGCCAACATC | |
| | AAGCAGGACGGCAGCGAGAAGTACTACGTGGACAGCGTGAAGGG | |
| | CAGGTTCACCATCAGCAGGGACAACGCCAAGAACAGCCTGTACC | |
| | TGCAGATGAACAGCCTGAGGGCCGAGGACACCGCCGTGTACTAC | |
| | TGCGCCAGGGAGGGCGGCCCCTACTACGACAGCAGCGGCTACTTC | |
| | GTGTACTACGGCATGGACGTGTGGGGCCAGGGCACCACCGTGAC | |
| | CGTGAGCAGC [SEQ ID NO: 246] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACGGCATGAGCTGGGTGAGGCAGGCCCCCGGCAAGTGCC | |
| | TGGAGTGGGTGGCCAACATCAAGCAGGACGGCAGCGAGAAGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGGCGGCCCCTACT | |
| | ACGACAGCAGCGGCTACTTCGTGTACTACGGCATGGACGTGTGGG | |
| | GCCAGGGCACCACCGTGACCGTGAGCAGCGGCGGCGGCGGCAGC | |
| | GGCGGCGGCGGCAGCGGCGGCGGCGGCAGCGGCGGCGGCGGCA | |
| | GCGACATCCAGATGACCCAGAGCCCCAGCACCCTGAGCGCCAGC | |
| | GTGGGCGACAGGGTGACCATCACCTGCAGGGCCAGCCAGAGCAT | |
| | CAGCAGCTGGCTGGCCTGGTACCAGCAGAAGCCCGGCAAGGCCC | |
| | CCAAGCTGCTGATCTACGACGCCAGCAGCCTGGAGAGCGGCGTG | |
| | CCCAGCAGGTTCAGCGGCAGCGGCAGCGGCACCGAGTTCACCCT | |
| | GACCATCAGCAGCCTGCAGCCCGACGACrrCGCCACCTACTACrG | |
| | CCAGCAGTACGAGAGCTTCCCCACCTTCGGCTGCGGCACCAAGGT | |
| | GGAGATCAAG [SEQ ID NO: 247] | |
| ADI-10177 | EVQLVESGGGLVQPGGSLRLSC | DIQMTQSPSTLSASVGDRVTITCR |
| [Ab2] | AASGFTFSSYWMSWVRQAPGK | ASQSISSWLAWYQQKPGKAPKL |
| | GLEWVANIKQDGSEKYYVDSV | LIYEASSLESGVPSRFSGSGSGTE |
| | KGRFTISRDNAKNSLYLQMNSL | FTLTISSLQPDDFATYYCQQLESY |
| | RAEDTAVYYCARPLNAGELDV | PLTFGGGTKVEIK [SEQ ID NO: 4] |
| | WGQGTMVTVSS [SEQ ID NO: 3] | CDR1: RASQSISSWLA [SEQ ID |
| | CDR1: FTFSSYWMS [SEQ ID | NO: 30] |
| | NO: 27](non-Kabat) or SYWMS | CDR2: EASSLES [SEQ ID NO: 31] |
| | [SEQ ID NO: 181] | CDR3: QQLESYPLT [SEQ ID |
| | CDR2: NIKQDGSEKYYVDSVKG | NO: 32] |
| | [SEQ ID NO: 28] | |
| | CDR3: ARPLNAGELDV [SEQ ID | |
| | NO: 29](non-Kabat) or | |
| | PLNAGELDV [SEQ ID NO: 436] | |
| scFv of | DIQMTQSPSTLSASVGDRVTITCRASQSISSWLAWYQQKPGKAPKLLI | |
| Ab2 | YEASSLESGVPSRFSGSGSGTEFTLTISSLQPDDFATYYCQQLESYPLT | |
| | FG [symbol] GTKVEIKGGGGSGGGGSGGGGSGGGGSEVQLVESGGGLVQP | |
| | GGSLRLSCAASGFTFSSYWMSWVRQAPGK [symbol] LEWVANIKQDGSEKY | |
| | YVDSVKGRFTISRDNAKNSLYLQMNSLRAEDTAVYYCARPLNAGEL | |
| | DVWGQGTMVTVSS [SEQ ID NO: 208] | |
| | EVQLVESGGGLVQPGGSLRLSCAASGFTFSSYWMSWVRQAPGK [symbol] LE | |
| | WVANIKQDGSEKYYVDSVKGRFTISRDNAKNSLYLQMNSLRAEDTA | |
| | VYYCARPLNAGELDVWGQGTMVTVSSGGGGSGGGGSGGGGSGG | |
| | GGSDIQMTQSPSTLSASVGDRVTITCRASQSISSWLAWYQQKPGKAP | |
| | KLLIYEASSLESGVPSRFSGSGSGTEFTLTISSLQPDDFATYYCQQLES | |
| | YPLTFG [symbol] GTKVEIK [SEQ ID NO: 209] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCCAGAGCATCA | |
| sequence | GCAGCTGGCTGGCCTGGTACCAGCAGAAGCCCGGCAAGGCCCCC | |
| of Ab2 | AAGCTGCTGATCTACGAGGCCAGCAGCCTGGAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGCTGGAGAGCTACCCCCTGACCTTCGGCTGCGGCACCAAGG | |
| | TGGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCCGGCAAGTGCCTGGAGTGGGTGGCCAAC | |
| | ATCAAGCAGGACGGCAGCGAGAAGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGCCCCTGAACGCCGGCGAGCTGGACGTGTGGGG | |
| | CCAGGGCACCATGGTGACCGTGAGCAGC [SEQ ID NO: 248] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAAGCAGGACGGCAGCGAGAAGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGCCCCTGAACGCCGGCG | |
| | AGCTGGACGTGTGGGGCCAGGGCACCATGGTGACCGTGAGCAGC | |
| | GGCGGCGGCGGCAGCGGCGGCGGCGGCAGCGGCGGCGGCGGCA | |
| | GCGGCGGCGGCGGCAGCGACATCCAGATGACCCAGAGCCCCAGC | |
| | ACCCTGAGCGCCAGCGTGGGCGACAGGGTGACCATCACCTGCAG | |
| | GGCCAGCCAGAGCATCAGCAGCTGGCTGGCCTGGTACCAGCAGA | |
| | AGCCCGGCAAGGCCCCCAAGCTGCTGATCTACGAGGCCAGCAGC | |
| | CTGGAGAGCGGCGTGCCCAGCAGGTTCAGCGGCAGCGGCAGCGG | |
| | CACCGAGTTCACCCTGACCATCAGCAGCCTGCAGCCCGACGACTT | |
| | CGCCACCTACTACTGCCAGCAGCTGGAGAGCTACCCCCTGACCTT | |
| | CGGCTGCGGCACCAAGGTGGAGATCAAG [SEQ ID NO: 249] | |
| ADI-11776 | EVQLLESGGGLVQPGGSLRLSC | DIQMTQSPSTLSASVGDRVTITCR |
| [Ab3] | AASGFTFSKYTMSWVRQAPGK | ASQSISSWLAWYQQKPGKAPKL |
| (H76) | GLEWVSAIVGSGESTYFADSVK | LIYKASSLESGVPSRFSGSGSGTE |
| | GRFTISRDNSKNTLYLQMNSLR | FTLTISSLQPDDFATYYCQQYDD |
| | AEDTAVYYCAREGGPYYDSSG | LPTFGGGTKVEIK [SEQ ID NO: 6] |
| | YFVYYGMDVWGQGTTVTVSS | CDR1: RASQSISSWLA [SEQ ID |
| | [SEQ ID NO: 5] | NO: 36] |
| | CDR1: FTFSKYTMS [SEQ ID | CDR2: KASSLES [SEQ ID NO: 37] |
| | NO: 33](non-Kabat) or KYTMS | or KASSLE [SEQ ID NO: 185](non- |
| | [SEQ ID NO: 183] | Kabat) |
| | CDR2: AIVGSGESTYFADSVKG | CDR3: QQYDDLPT [SEQ ID |
| | [SEQ ID NO: 34] | NO: 38] |
| | CDR3: | |
| | AREGGPYYDSSGYFVYYGMDV | |
| | [SEQ ID NO: 35](non-Kabat) or | |
| | EGGPYYDSSGYFVYYGMDV | |
| | [SEQ ID NO: 184] | |
| scFv of | DIQMTQSPSTLSASVGDRVTITCRASQSISSWLAWYQQKPGKAPKLLI | |
| Ab3 | YKASSLESGVPSRFSGSGSGTEFTLTISSLQPDDFATYYCQQYDDLPT | |
| | FG [symbol] GTKVEIKGGGGSGGGGSGGGGSGGGGSEVQLLESGGGLVQP | |
| | GGSLRLSCAASGFTFSKYTMSWVRQAPGK [symbol] LEWVSAIVGSGESTYF | |
| | ADSVKGRFTISRDNSKNTLYLQMNSLRAEDTAVYYCAREGGPYYDS | |
| | SGYFVYYGMDVWGQGTTVTVSS | |
| | [SEQ ID NO: 198] | |
| | EVQLLESGGGLVQPGGSLRLSCAASGFTFSKYTMSWVRQAPGK [symbol] LE | |
| | WVSAIVGSGESTYFADSVKGRFTISRDNSKNTLYLQMNSLRAEDTAV | |
| | YYCAREGGPYYDSSGYFVYYGMDVWGQGTTVTVSSGGGGSGGGG | |
| | SGGGGSGGGGSDIQMTQSPSTLSASVGDRVTITCRASQSISSWLAW | |
| | YQQKPGKAPKLLIYKASSLESGVPSRFSGSGSGTEFTLTISSLQPDDFA | |
| | TYYCQQYDDLPTFG [symbol] GTKVEIK [SEQ ID NO: 210] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCCAGAGCATCA | |
| sequence | GCAGCTGGCTGGCCTGGTACCAGCAGAAGCCCGGCAAGGCCCCC | |
| of Ab3 | AAGCTGCTGATCTACAAGGCCAGCAGCCTGGAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGTACGACGACCTGCCCACCTTCGGCTGCGGCACCAAGGTGG | |
| | AGATCAAGGGCGGCGGCGGCAGCGGCGGCGGCGGCAGCGGCGGC | |
| | GGCGGCAGCGGCGGCGGCGGCAGCGAGGTGCAGCTGCTGGAGAG | |
| | CGGCGGCGGCCTGGTGCAGCCCGGCGGCAGCCTGAGGCTGAGCT | |
| | GCGCCGCCAGCGGCTTCACCTTCAGCAAGTACACCATGAGCTGGG | |
| | TGAGGCAGGCCCCCGGCAAGTGCCTGGAGTGGGTGAGCGCCATC | |
| | GTGGGCAGCGGCGAGAGCACCTACTTCGCCGACAGCGTGAAGGG | |
| | CAGGTTCACCATCAGCAGGGACAACAGCAAGAACACCCTGTACC | |
| | TGCAGATGAACAGCCTGAGGGCCGAGGACACCGCCGTGTACTAC | |
| | TGCGCCAGGGAGGGCGGCCCCTACTACGACAGCAGCGGCTACTTC | |
| | GTGTACTACGGCATGGACGTGTGGGGCCAGGGCACCACCGTGAC | |
| | CGTGAGCAGC [SEQ ID NO: 250] | |
| | GAGGTGCAGCTGCTGGAGAGCGGCGGCGGCCTGGTGCAGCCCGG | |
| | CGGCAGCCTGAGGCTGAGCTGCGCCGCCAGCGGCTTCACCTTCAG | |
| | CAAGTACACCATGAGCTGGGTGAGGCAGGCCCCCGGCAAGTGCC | |
| | TGGAGTGGGTGAGCGCCATCGTGGGCAGCGGCGAGAGCACCTAC | |
| | TTCGCCGACAGCGTGAAGGGCAGGTTCACCATCAGCAGGGACAA | |
| | CAGCAAGAACACCCTGTACCTGCAGATGAACAGCCTGAGGGCCG | |
| | AGGACACCGCCGTGTACTACTGCGCCAGGGAGGGCGGCCCCTACT | |
| | ACGACAGCAGCGGCTACTTCGTGTACTACGGCATGGACGTGTGGG | |
| | GCCAGGGCACCACCGTGACCGTGAGCAGCGGCGGCGGCGGCAGC | |
| | GGCGGCGGCGGCAGCGGCGGCGGCGGCAGCGGCGGCGGCGGCA | |
| | GCGACATCCAGATGACCCAGAGCCCCAGCACCCTGAGCGCCAGC | |
| | GTGGGCGACAGGGTGACCATCACCTGCAGGGCCAGCCAGAGCAT | |
| | CAGCAGCTGGCTGGCCTGGTACCAGCAGAAGCCCGGCAAGGCCC | |
| | CCAAGCTGCTGATCTACAAGGCCAGCAGCCTGGAGAGCGGCGTG | |
| | CCCAGCAGGTTCAGCGGCAGCGGCAGCGGCACCGAGTTCACCCT | |
| | GACCATCAGCAGCCTGCAGCCCGACGACTTCGCCACCTACTACTG | |
| | CCAGCAGTACGACGACCTGCCCACCTTCGGCTGCGGCACCAAGGT | |
| | GGAGATCAAG [SEQ ID NO: 251] | |
| ADI-11801 | QVQLVQSGAEVKKPGASVKVS | DIVMTQSPLSLPVTPGEPASISCR |
| | CKASGYTFSDYYMHWVRQAPG | SSQSLLYSNGYNYLDWYLQKPG |
| [Ab4] | QGLEWMGMINPSWGSTSYAQK | QSPQLLIYLGSNRASGVPDRFSGS |
| | FQGRVTMTRDTSTSTVYMELSS | GSGTDFTLKISRVEAEDVGVYYC |
| | LRSEDTAVYYCAREAADGFVGE | MQDVALPITFGGGTKVEIK [SEQ |
| | RYFDLWGRGTLVTVSS [SEQ ID | ID NO: 8] |
| | NO: 7] | CDR1: RSSQSLLYSNGYNYLD |
| | CDR1: YTFSDYYMH [SEQ ID | [SEQ ID NO: 42] |
| | NO: 39](non-Kabat) or DYYMH | CDR2: LGSNRAS [SEQ ID NO: 43] |
| | [SEQ ID NO: 437] | CDR3: MQDVALPIT [SEQ ID |
| | CDR2: MINPSWGSTSYAQKFQG | NO: 44] |
| | [SEQ ID NO: 40] | |
| | CDR3: AREAADGFVGERYFDL | |
| | [SEQ ID NO: 41](non-Kabat) or | |
| | EAADGFVGERYFDL [SEQ ID | |
| | NO: 438] | |
| scFv of | DIVMTQSPLSLPVTPGEPASISCRSSQSLLYSNGYNYLDWYLQKPGQS | |
| Ab4 | PQLLIYLGSNRASGVPDRFSGSGSGTDFTLKISRVEAEDVGVYYCMQ | |
| | DVALPITFG [symbol] GTKVEIKGGGGSGGGGSGGGGSGGGGSQVQLVQSG | |
| | AEVKKPGASVKVSCKASGYTFSDYYMHWVRQAPGQ [symbol] LEWMGMIN | |
| | PSWGSTSYAQKFQGRVTMTRDTSTSTVYMELSSLRSEDTAVYYCAR | |
| | EAADGFVGERYFDLWGRGTLVTVSS [SEQ ID NO: 211] | |
| | QVQLVQSGAEVKKPGASVKVSCKASGYTFSDYYMHWVRQAPGQ [symbol] L | |
| | EWMGMINPSWGSTSYAQKFQGRVTMTRDTSTSTVYMELSSLRSEDT | |
| | AVYYCAREAADGFVGERYFDLWGRGTLVTVSSGGGGSGGGGSGG | |
| | GGSGGGGSDIVMTQSPLSLPVTPGEPASISCRSSQSLLYSNGYNYLD | |
| | WYLQKPGQSPQLLIYLGSNRASGVPDRFSGSGSGTDFTLKISRVEAED | |
| | VGVYYCMQDVALPITFG [symbol] GTKVEIK [SEQ ID NO: 212] | |
| Exemplary | GACATCGTGATGACCCAGAGCCCCCTGAGCCTGCCCGTGACCCCC | |
| nucleotide | GGCGAGCCCGCCAGCATCAGCTGCAGGAGCAGCCAGAGCCTGCT | |
| sequence | GTACAGCAACGGCTACAACTACCTGGACTGGTACCTGCAGAAGCC | |
| of Ab4 | CGGCCAGAGCCCCCAGCTGCTGATCTACCTGGGCAGCAACAGGG | |
| scFv | CCAGCGGCGTGCCCGACAGGTTCAGCGGCAGCGGCAGCGGCACC | |
| | GACTTCACCCTGAAGATCAGCAGGGTGGAGGCCGAGGACGTGGG | |
| | CGTGTACTACTGCATGCAGGACGTGGCCCTGCCCATCACCTTCGG | |
| | CTGCGGCACCAAGGTGGAGATCAAGGGCGGCGGCGGCAGCGGCG | |
| | GCGGCGGCAGCGGCGGCGGCGGCAGCGGCGGCGGCGGCAGCCAG | |
| | GTGCAGCTGGTGCAGAGCGGCGCCGAGGTGAAGAAGCCCGGCGC | |
| | CAGCGTGAAGGTGAGCTGCAAGGCCAGCGGCTACACCTTCAGCG | |
| | ACTACTACATGCACTGGGTGAGGCAGGCCCCCGGCCAGTGCCTGG | |
| | AGTGGATGGGCATGATCAACCCCAGCTGGGGCAGCACCAGCTAC | |
| | GCCCAGAAGTTCCAGGGCAGGGTGACCATGACCAGGGACACCAG | |
| | CACCAGCACCGTGTACATGGAGCTGAGCAGCCTGAGGAGCGAGG | |
| | ACACCGCCGTGTACTAGTGCGCCAGGGAGGCCGCCGACGGCTTCG | |
| | TGGGCGAGAGGTACTTCGACCTGTGGGGCAGGGGCACCCTGGTG | |
| | ACCGTGAGCAGC [SEQ ID NO: 252] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCA | |
| | GCGACTACTACATGCACTGGGTGAGGCAGGCCCCCGGCCAGTGCC | |
| | TGGAGTGGATGGGCATGATCAACCCCAGCTGGGGCAGCACCAGC | |
| | TACGCCCAGAAGTTCCAGGGCAGGGTGACCATGACCAGGGACAC | |
| | CAGCACCAGCACCGTGTACATGGAGCTGAGCAGCCTGAGGAGCG | |
| | AGGACACCGCCGTGTACTACTGCGCCAGGGAGGCCGCCGACGGC | |
| | TTCGTGGGCGAGAGGTACTTCGACCTGTGGGGCAGGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCGTGATGA | |
| | CCCAGAGCCCCCTGAGCCTGCCCGTGACCCCCGGCGAGCCCGCCA | |
| | GCATCAGCTGCAGGAGCAGCCAGAGCCTGCTGTACAGCAACGGC | |
| | TACAACTACCTGGACTGGTACCTGCAGAAGCCCGGCCAGAGCCCC | |
| | CAGCTGCTGATCTACCTGGGCAGCAACAGGGCCAGCGGCGTGCCC | |
| | GACAGGTTCAGCGGCAGCGGCAGCGGCACCGACTTCACCCTGAA | |
| | GATCAGCAGGGTGGAGGCCGAGGACGTGGGCGTGTACTACTGCA | |
| | TGCAGGACGTGGCCCTGCCCATCACCTTCGGCTGCGGCACCAAGG | |
| | TGGAGATCAAG [SEQ ID NO: 253] | |
| ADI-11807 | EVQLVESGGGLVQPGGSLRLSC | DIQMTQSPSTLSASVGDRVTITCR |
| [Ab5] | AASGFTFGSYWMSWVRQAPGK | ASQSISSWLAWYQQKPGKAPKL |
| (I07) | GLEWVATIKQDGSEKSYVDSVK | LIYEASSLESGVPSRFSGSGSGTE |
| | GRFTISRDNAKNSLYLQMNSLR | FTLTISSLQPDDFATYYCQQSQSY |
| | AEDTAVYYCARPLNAGELDVW | PPITFGGGTKVEIK [SEQ ID |
| | GQGTMVTVSS [SEQ ID NO: 9] | NO: 10] |
| | CDR1: FTFGSYWMS [SEQ ID | CDR1: RASQSISSWLA [SEQ ID |
| | NO: 45](non-Kabat) or SYWMS | NO: 48] |
| | [SEQ ID NO: 181] | CDR2: EASSLES [SEQ ID NO: 49] |
| | CDR2: TIKQDGSEKSYVDSVKG | CDR3: QQSQSYPPIT [SEQ ID |
| | [SEQ ID NO: 46] | NO: 50] |
| | CDR3: ARPLNAGELDV [SEQ ID | |
| | NO: 47](non-Kabat) or | |
| | RPLNAGELDV [SEQ ID NO: 182] | |
| scFv of | I07 scFv | |
| Ab5 | DIQMTQSPSTLSASVGDRVTITCRASQSISSWLAWYQQKPGKAPKLLI | |
| | YEASSLESGVPSRFSGSGSGTEFTLTISSLQPDDFATYYCQQSQSYPPI | |
| | TFG [symbol] GTKVEIKGGGGSGGGGSGGGGSGGGGSEVQLVESGGGLVQ | |
| | PGGSLRLSCAASGFTFGSYWMSWVRQAPGK [symbol] LEWVATIKQDGSEKS | |
| | YVDSVKGRFTISRDNAKNSLYLQMNSLRAEDTAVYYCARPLNAGEL | |
| | DVWGQGTMVTVSS [SEQ ID NO: 188] | |
| | EVQLVESGGGLVQPGGSLRLSCAASGFTFGSYWMSWVRQAPGK [symbol] LE | |
| | WVATIKQDGSEKSYVDSVKGRFTISRDNAKNSLYLQMNSLRAEDTA | |
| | VYYCARPLNAGELDVWGQGTMVTVSSGGGGSGGGGSGGGGSGG | |
| | GGSDIQMTQSPSTLSASVGDRVTITCRASQSISSWLAWYQQKPGKAP | |
| | KLLIYEASSLESGVPSRFSGSGSGTEFTLTISSLQPDDFATYYCQQSQS | |
| | YPPITFG [symbol] GTKVEIK [SEQ ID NO: 213] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCCAGAGCATCA | |
| sequence | GCAGCTGGCTGGCCTGGTACCAGCAGAAGCCCGGCAAGGCCCCC | |
| of Ab5 | AAGCTGCTGATCTACGAGGCCAGCAGCCTGGAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGAGCCAGAGCTACCCCCCCATCACCTTCGGCTGCGGCACCA | |
| | AGGTGGAGATCAAGGGCGGCGGCGGCAGCGGCGGCGGCGGCAGC | |
| | GGCGGCGGCGGCAGCGGCGGCGGCGGCAGCGAGGTGCAGCTGGT | |
| | GGAGAGCGGCGGCGGCCTGGTGCAGCCCGGCGGCAGCCTGAGGC | |
| | TGAGCTGCGCCGCCAGCGGCTTCACCTTCGGCAGCTACTGGATGA | |
| | GCTGGGTGAGGCAGGCCCCCGGCAAGTGCCTGGAGTGGGTGGCC | |
| | ACCATCAAGCAGGACGGCAGCGAGAAGAGCTACGTGGACAGCGT | |
| | GAAGGGCAGGTTCACCATCAGCAGGGACAACGCCAAGAACAGCC | |
| | TGTACCTGCAGATGAACAGCCTGAGGGCCGAGGACACCGCCGTG | |
| | TACTACTGCGCCAGGCCCCTGAACGCCGGCGAGCTGGACGTGTGG | |
| | GGCCAGGGCACCATGOTGACCGTGAGCAGC [SEQ ID NO: 254] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCGG | |
| | CAGCTACTGGATGAGCTGGGTGAGGCAGGCCCCCGGCAAGTGCC | |
| | TGGAGTGGGTGGCCACCATCAAGCAGGACGGCAGCGAGAAGAGC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGCCCCTGAACGCCGGCG | |
| | AGCTGGACGTGTGGGGCCAGGGCACCATGGTGACCGTGAGCAGC | |
| | GGCGGCGGCGGCAGCGGCGGCGGCGGCAGCGGCGGCGGCGGCA | |
| | GCGGCGGCGGCGGCAGCGACATCCAGATGACCCAGAGCCCCAGC | |
| | ACCCTGAGCGCCAGCGTGGGCGACAGGGTGACCATCACCTGCAG | |
| | GGCCAGCCAGAGCATCAGCAGCTGGCTGGCCTGGTACCAGCAGA | |
| | AGCCCGGCAAGGCCCCCAAGCTGCTGATCTACGAGGCCAGCAGC | |
| | CTGGAGAGCGGCGTGCCCAGCAGGTTCAGCGGCAGCGGCAGCGG | |
| | CACCGAGTTCACCCTGACCATCAGCAGCCTGCAGCCCGACGACTT | |
| | CGCCACCTACTACTGCCAGCAGAGCCAGAGCTACCCCCCCATCAC | |
| | CTTCGGCTGCGGCACCAAGGTGGAGATCAAG [SEQ ID NO: 255] | |
| ADI-11809 | EVQLVESGGGLVQPGGSLRLSC | DIQMTQSPSTLSASVGDRVTITCR |
| [Ab6] | AASGFTFPSYWMSWVRQAPGK | ASQSISSWLAWYQQKPGKAPKL |
| | GLEWVATIKRDGSEKGYVDSV | LIYEASSLESGVPSRFSGSGSGTE |
| | KGRFTISRDNAKNSLYLQMNSL | FTLTISSLQPDDFATYYCQQSQSY |
| | RAEDTAVYYCARPLNAGELDV | PPITFGGGTKVEIK [SEQ ID |
| | WGQGTMVTVSS [SEQ ID | NO: 12] |
| | NO: 11] | CDR1: RASQSISSWLA [SEQ ID |
| | CDR1: FTFPSYWMS [SEQ ID | NO: 54] |
| | NO: 51](non-Kabat) or SYWMS | CDR2: EASSLES [SEQ ID NO: 55] |
| | [SEQ ID NO: 181] | CDR3: QQSQSYPPIT [SEQ ID |
| | CDR2: TIKRDGSEKGYVDSVKG | NO: 56] |
| | [SEQ ID NO: 52] | |
| | CDR3: ARPLNAGELDV [SEQ ID | |
| | NO: 53](non-Kabat) or | |
| | PLNAGELDV [SEQ ID NO: 439] | |
| scFv of | DIQMTQSPSTLSASVGDRVTITCRASQSISSWLAWYQQKPGKAPKLLI | |
| Ab6 | YEASSLESGVPSRFSGSGSGTEFTLTISSLQPDDFATYYCQQSQSYPPI | |
| | TFG [symbol] GTKVEIKGGGGSGGGGSGGGGSGGGGSEVQLVESGGGLVQ | |
| | PGGSLRLSCAASGFTFPSYWMSWVRQAPGK [symbol] LEWVATIKRDGSEKG | |
| | YVDSVKGRFTISRDNAKNSLYLQMNSLRAEDTAVYYCARPLNAGEL | |
| | DVWGQGTMVTVSS [SEQ ID NO: 214] | |
| | EVQLVESGGGLVQPGGSLRLSCAASGFTFPSYWMSWVRQAPGK [symbol] LE | |
| | WVATIKRDGSEKGYVDSVKGRFTISRDNAKNSLYLQMNSLRAEDTA | |
| | VYYCARPLNAGELDVWGQGTMVTVSSGGGGSGGGGSGGGGSGG | |
| | GGSDIQMTQSPSTLSASVGDRVTITCRASQSISSWLAWYQQKPGKAP | |
| | KLLIYEASSLESGVPSRFSGSGSGTEFTLTISSLQPDDFATYYCQQSQS | |
| | YPPITFG [symbol] GTKVEIK [SEQ ID NO: 215] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCCAGAGCATCA | |
| sequence | GCAGCTGGCTGGCCTGGTACCAGCAGAAGCCCGGCAAGGCCCCC | |
| of Ab6 | AAGCTGCTGATCTACGAGGCCAGCAGCCTGGAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGAGCCAGAGCTACCCCCCCATCACCTTCGGCTGCGGCACCA | |
| | AGGTGGAGATCAAGGGCGGCGGCGGCAGCGGCGGCGGCGGCAGC | |
| | GGCGGCGGCGGCAGCGGCGGCGGCGGCAGCGAGGTGCAGCTGGT | |
| | GGAGAGCGGCGGCGGCCTGGTGCAGCCCGGCGGCAGCCTGAGGC | |
| | TGAGCTGCGCCGCCAGCGGCTTCACCTTCCCCAGCTACTGGATGA | |
| | GCTGGGTGAGGCAGGCCCCCGGCAAGTGCCTGGAGTGGGTGGCC | |
| | ACCATCAAGAGGGACGGCAGCGAGAAGGGCTACGTGGACAGCGT | |
| | GAAGGGCAGGTTCACCATCAGCAGGGACAACGCCAAGAACAGCC | |
| | TGTACCTGCAGATGAACAGCCTGAGGGCCGAGGACACCGCCGTG | |
| | TACTACTGCGCCAGGCCCCTGAACGCCGGCGAGCTGGACGTGTGG | |
| | GGCCAGGGCACCATGGTGACCGTGAGCAGC [SEQ ID NO: 256] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCCC | |
| | CAGCTACTGGATGAGCTGGGTGAGGCAGGCCCCCGGCAAGTGCC | |
| | TGGAGTGGGTGGCCACCATCAAGAGGGACGGCAGCGAGAAGGGC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGCCCCTGAACGCCGGCG | |
| | AGCTGGACGTGTGGGGCCAGGGCACCATGGTGACCGTGAGCAGC | |
| | GGCGGCGGCGGCAGCGGCGGCGGCGGCAGCGGCGGCGGCGGCA | |
| | GCGGCGGCGGCGGCAGCGACATCCAGATGACCCAGAGCCCCAGC | |
| | ACCCTGAGCGCCAGCGTGGGCGACAGGGTGACCATCACCTGCAG | |
| | GGCCAGCCAGAGCATCAGCAGCTGGCTGGCCTGGTACCAGCAGA | |
| | AGCCCGGCAAGGCCCCCAAGCTGCTGATCTACGAGGCCAGCAGC | |
| | CTGGAGAGCGGCGTGCCCAGCAGGTTCAGCGGCAGCGGCAGCGG | |
| | CACCGAGTTCACCCTGACCATCAGCAGCCTGCAGCCCGACGACTT | |
| | CGCCACCTACTACTGCCAGCAGAGCCAGAGCTACCCCCCCATCAC | |
| | CTTCGGCTGCGGCACCAAGGTGGAGATCAAG [SEQ ID NO: 257] | |
| ADI-11815 | QVQLVQSGAEVKKPGASVKVS | DIQMTQSPSSVSASVGDRVTITC |
| [Ab7] | CKASGYTFGTYYMHWVRQAPG | RASQGIDSWLAWYQQKPGKAPK |
| | QGLEWMGIINPSRGSTVYAQKF | LLIYAASSLQSGVPSRFSGSGSGT |
| | QGRVTMTRDTSTSTVYMELSSL | DFTLTISSLQPEDFATYYCQQAHS |
| | RSEDTAVYYCARGAGYDDEDM | YPLTFGGGTKVEIK [SEQ ID |
| | DVWGKGTTVTVSS [SEQ ID | NO: 14] |
| | NO: 13] | CDR1: RASQGIDSWLA [SEQ ID |
| | CDR1: YTFGTYYMH [SEQ ID | NO: 60] |
| | NO: 57](non-Kabat) or TYYMH | CDR2: AASSLQS [SEQ ID NO: 61] |
| | [SEQ ID NO: 440] | CDR3: QQAHSYPLT [SEQ ID |
| | CDR2: IINPSRGSTVYAQKFQG | NO: 62] |
| | [SEQ ID NO: 58] | |
| | CDR3: ARGAGYDDEDMDV | |
| | [SEQ ID NO: 59](non-Kabat) or | |
| | GAGYDDEDMDV [SEQ ID | |
| | NO: 441] | |
| scFv of | DIQMTQSPSSVSASVGDRVTITCRASQGIDSWLAWYQQKPGKAPKLL | |
| Ab7 | IYAASSLQSGVPSRFSGSGSGTDFTLTISSLQPEDFATYYCQQAHSYPL | |
| | TFG [symbol] GTKVEIKGGGGSGGGGSGGGGSGGGGSQVQLVQSGAEVKK | |
| | PGASVKVSCKASGYTFGTYYMHWVRQAPGQ [symbol] LEWMGIINPSRGSTV | |
| | YAQKFQGRVTMTRDTSTSTVYMELSSLRSEDTAVYYCARGAGYDD | |
| | EDMDVWGKGTTVTVSS [SEQ ID NO: 216] | |
| | QVQLVQSGAEVKKPGASVKVSCKASGYTFGTYYMHWVRQAPGQ [symbol] | |
| | LEWMGIINPSRGSTVYAQKFQGRVTMTRDTSTSTVYMELSSLRSEDT | |
| | AVYYCARGAGYDDEDMDVWGKGTTVTVSSGGGGSGGGGSGGGG | |
| | SGGGGSDIQMTQSPSSVSASVGDRVTITCRASQGIDSWLAWYQQKP | |
| | GKAPKLLIYAASSLQSGVPSRFSGSGSGTDFTLTISSLQPEDFATYYCQ | |
| | QAHSYPLTFG [symbol] GTKVEIK [SEQ ID NO: 217] | |
| Exemplary | GACATCCAGATGACCCAGAGCCCCAGCAGCGTGAGCGCCAGCGT | |
| nucleotide | GGGCGACAGGGTGACCATCACCTGCAGGGCCAGCCAGGGCATCG | |
| sequence | ACAGCTGGCTGGCCTGGTACCAGCAGAAGCCCGGCAAGGCCCCC | |
| of Ab7 | AAGCTGCTGATCTACGCCGCCAGCAGCCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGCCCACAGCTACCCCCTGACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCCAGGTGCAGCTGGTGCA | |
| | GAGCGGCGCCGAGGTGAAGAAGCCCGGCGCCAGCGTGAAGGTGA | |
| | GCTGCAAGGCCAGCGGCTACACCTTCGGCACCTACTACATGCACT | |
| | GGGTGAGGCAGGCCCCCGGCCAGTGCCTGGAGTGGATGGGCATC | |
| | ATCAACCCCAGCAGGGGCAGCACCGTGTACGCCCAGAAGTTCCA | |
| | GGGCAGGGTGACCATGACCAGGGACACCAGCACCAGCACCGTGT | |
| | ACATGGAGCTGAGCAGCCTGAGGAGCGAGGACACCGCCGTGTAC | |
| | TACTGCGCCAGGGGCGCCGGCTACGACGACGAGGACATGGACGT | |
| | GTGGGGCAAGGGCACCACCGTGACCGTGAGCAGC [SEQ ID | |
| | NO: 258] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCG | |
| | GCACCTACTACATGCACTGGGTGAGGCAGGCCCCCGGCCAGTGCC | |
| | TGGAGTGGATGGGCATCATCAACCCCAGCAGGGGCAGCACCGTG | |
| | TACGCCCAGAAGTTCCAGGGCAGGGTGACCATGACCAGGGACAC | |
| | CAGCACCAGCACCGTGTACATGGAGCTGAGCAGCCTGAGGAGCG | |
| | AGGACACCGCCGTGTACTACTGCGCCAGGGGCGCCGGCTACGAC | |
| | GACGAGGACATGGACGTGTGGGGCAAGGGCACCACCGTGACCGT | |
| | GAGCAGCGGCGGCGGCGGCAGCGGCGGCGGCGGCAGCGGCGGC | |
| | GGCGGCAGCGGCGGCGGCGGCAGCGACATCCAGATGACCCAGAG | |
| | CCCCAGCAGCGTGAGCGCCAGCGTGGGCGACAGGGTGACCATCA | |
| | CCTGCAGGGCCAGCCAGGGCATCGACAGCTGGCTGGCCTGGTACC | |
| | AGCAGAAGCCCGGCAAGGCCCCCAAGCTGCTGATCTACGCCGCC | |
| | AGCAGCCTGCAGAGCGGCGTGCCCAGCAGGTTCAGCGGCAGCGG | |
| | CAGCGGCACCGACTTCACCCTGACCATCAGCAGCCTGCAGCCCGA | |
| | GGACTTCGCCACCTACTACTGCCAGCAGGCCCACAGCTACCCCCT | |
| | GACCTTCGGCTGCGGCACCAAGGTGGAGATCAAG [SEQ ID NO: | |
| | 259] | |
| ADI-11819 | EVQLVESGGGLVKPGGSLRLSC | DIQMTQSPSTLSASVGDRVTITCR |
| [Ab8] | AASGFTFSSYAMSWVRQAPGK | ASNSISSWLAWYQQKPGKAPKL |
| | GLEWVSSISSSSEGIYYADSVKG | LIYEASSTKSGVPSRFSGSGSGTE |
| | RFTISRDNAKNSLYLQMNSLRA | FTLTISSLQPDDFATYYCQQYDD |
| | EDTAVYYCAREGGPYYDSSGYF | LPTFGGGTKVEIK [SEQ ID |
| | VYYGMDVWGQGTTVTVSS | NO: 16] |
| | [SEQ ID NO: 15] | CDR1: RASNSISSWLA [SEQ ID |
| | CDR1: FTFSSYAMS [SEQ ID | NO: 66] |
| | NO: 63](non-Kabat) or SYAMS | CDR2: EASSTKS [SEQ ID NO: 67] |
| | [SEQ ID NO: 442] | CDR3: QQYDDLPT [SEQ ID |
| | CDR2: SISSSSEGIYYADSVKG | NO: 68] |
| | [SEQ ID NO: 64] | |
| | CDR3: | |
| | AREGGPYYDSSGYFVYYGMDV | |
| | [SEQ ID NO: 65](non-Kabat) or | |
| | EGGPYYDSSGYFVYYGMDV | |
| | [SEQ ID NO: 443] | |
| scFv of | DIQMTQSPSTLSASVGDRVTITCRASNSISSWLAWYQQKPGKAPKLLI | |
| Ab8 | YEASSTKSGVPSRFSGSGSGTEFTLTISSLQPDDFATYYCQQYDDLPT | |
| | FG [symbol] GTKVEIKGGGGSGGGGSGGGGSGGGGSEVQLVESGGGLVKP | |
| | GGSLRLSCAASGFTFSSYAMSWVRQAPGK [symbol] LEWVSSISSSSEGIYYA | |
| | DSVKGRFTISRDNAKNSLYLQMNSLRAEDTAVYYCAREGGPYYDSS | |
| | GYFVYYGMDVWGQGTTVTVSS [SEQ ID NO: 218] | |
| | EVQLVESGGGLVKPGGSLRLSCAASGFTFSSYAMSWVRQAPGK [symbol] LE | |
| | WVSSISSSSEGIYYADSVKGRFTISRDNAKNSLYLQMNSLRAEDTAV | |
| | YYCAREGGPYYDSSGYFVYYGMDVWGQGTTVTVSSGGGGSGGGG | |
| | SGGGGSGGGGSDIQMTQSPSTLSASVGDRVTITCRASNSISSWLAW | |
| | YQQKPGKAPKLLIYEASSTKSGVPSRFSGSGSGTEFTLTISSLQPDDFA | |
| | TYYCQQYDDLPTFG [symbol] GTKVEIK [SEQ ID NO: 219] | |
| Exemplary | GACATCCAGATGACCCAGAGCCCCAGCACCCTGAGCGCCAGCGT | |
| nucleotide | GGGCGACAGGGTGACCATCACCTGCAGGGCCAGCAACAGCATCA | |
| sequence | GCAGCTGGCTGGCCTGGTACCAGCAGAAGCCCGGCAAGGCCCCC | |
| of Ab8 | AAGCTGCTGATCTACGAGGCCAGCAGCACCAAGAGCGGCGTGCC | |
| scFv | CAGCAGGTTCAGCGGCAGCGGCAGCGGCACCGAGTTCACCCTGA | |
| | CCATCAGCAGCCTGCAGCCCGACGACTTCGCCACCTACTACTGCC | |
| | AGCAGTACGACGACCTGCCCACCTTCGGCTGCGGCACCAAGGTGG | |
| | AGATCAAGGGCGGCGGCGGCAGCGGCGGCGGCGGCAGCGGCGGC | |
| | GGCGGCAGCGGCGGCGGCGGCAGCGAGGTGCAGCTGGTGGAGAG | |
| | CGGCGGCGGCCTGGTGAAGCCCGGCGGCAGCCTGAGGCTGAGCT | |
| | GCGCCGCCAGCGGCTTCACCTTCAGCAGCTACGCCATGAGCTGGG | |
| | TGAGGCAGGCCCCCGGCAAGTGCCTGGAGTGGGTGAGCAGCATC | |
| | AGCAGCAGCAGCGAGGGCATCTACTACGCCGACAGCGTGAAGGG | |
| | CAGGTTCACCATCAGCAGGGACAACGCCAAGAACAGCCTGTACC | |
| | TGCAGATGAACAGCCTGAGGGCCGAGGACACCGCCGTGTACTAC | |
| | TGCGCCAGGGAGGGCGGCCCCTACTACGACAGCAGCGGCTACTTC | |
| | GTGTACTACGGCATGGACGTGTGGGGCCAGGGCACCACCGTGAC | |
| | CGTGAGCAGC [SEQ ID NO: 260] | |
| | GAGGTGCAGCTGGTGGAGAGCGGCGGCGGCCTGGTGAAGCCCGG | |
| | CGGCAGCCTGAGGCTGAGCTGCGCCGCCAGCGGCTTCACCTTCAG | |
| | CAGCTACGCCATGAGCTGGGTGAGGCAGGCCCCCGGCAAGTGCC | |
| | TGGAGTGGGTGAGCAGCATCAGCAGCAGCAGCGAGGGCATCTAC | |
| | TACGCCGACAGCGTGAAGGGCAGGITCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGGCGGCCCCTACT | |
| | ACGACAGCAGCGGCTACTTCGTGTACTACGGCATGGACGTGTGGG | |
| | GCCAGGGCACCACCGTGACCGTGAGCAGCGGCGGCGGCGGCAGC | |
| | GGCGGCGGCGGCAGCGGCGGCGGCGGCAGCGGCGGCGGCGGCA | |
| | GCGACATCCAGATGACCCAGAGCCCCAGCACCCTGAGCGCCAGC | |
| | GTGGGCGACAGGGTGACCATCACCTGCAGGGCCAGCAACAGCAT | |
| | CAGCAGCTGGCTGGCCTGGTACCAGCAGAAGCCCGGCAAGGCCC | |
| | CCAAGCTGCTGATCTACGAGGCCAGCAGCACCAAGAGCGGCGTG | |
| | CCCAGCAGGTTCAGCGGCAGCGGCAGCGGCACCGAGTTCACCCT | |
| | GACCATCAGCAGCCTGCAGCCCGACGACTTCGCCACCTACTACTG | |
| | CCAGCAGTACGACGACCTGCCCACCTTCGGCTGCGGCACCAAGGT | |
| | GGAGATCAAG [SEQ ID NO: 261] | |
| ADI-11830 | EVQLVESGGGLVQPGGSLRLSC | DIQMTQSPSSLSASVGDRVTITCR |
| [Ab9] | AASGFTFSSYWMSWVRQAPGK | ASQVIYSYLNWYQQKPGKAPKL |
| | GLEWVANINTDGSEVYYVDSV | LIYAASSLKSGVPSRFSGSGSGTD |
| | KGRFTISRDNAKNSLYLQMNSL | FTLTISSLQPEDFATYYCQQVYD |
| | RAEDTAVYYCARDVGPGIAYQ | TPLTFGGGTKVEIK [SEQ ID |
| | GHFDYWGQGTLVTVSS [SEQ ID | NO: 18] |
| | NO: 17] | CDR1: RASQVIYSYLN [SEQ ID |
| | CDR1: FTFSSYWMS [SEQ ID | NO: 72] |
| | NO: 69](non-Kabat) or SYWMS | CDR2: AASSLKS [SEQ ID NO: 73] |
| | [SEQ ID NO: 181] | CDR3: QQVYDTPLT [SEQ ID |
| | CDR2: NINTDGSEVYYVDSVKG | NO: 74] |
| | [SEQ ID NO: 70] | |
| | CDR3: ARDVGPGIAYQGHFDY | |
| | [SEQ ID NO: 71](non-Kabat) or | |
| | DVGPGIAYQGHFDY [SEQ ID | |
| | NO: 444] | |
| scFv of | DIQMTQSPSSLSASVGDRVTITCRASQVIYSYLNWYQQKPGKAPKLLI | |
| Ab9 | YAASSLKSGVPSRFSGSGSGTDFTLTISSLQPEDFATYYCQQVYDTPL | |
| | TFG [symbol] GTKVEIKGGGGSGGGGSGGGGSGGGGSEVQLVESGGGLVQ | |
| | PGGSLRLSCAASGFTFSSYWMSWVRQAPGK [symbol] LEWVANINTDGSEVY | |
| | YVDSVKGRFTISRDNAKNSLYLQMNSLRAEDTAVYYCARDVGPGIA | |
| | YQGHFDYWGQGTLVTVSS [SEQ ID NO: 220] | |
| | EVQLVESGGGLVQPGGSLRLSCAASGFTFSSYWMSWVRQAPGK [symbol] LE | |
| | WVANINTDGSEVYYVDSVKGRFTISRDNAKNSLYLQMNSLRAEDTA | |
| | VYYCARDVGPGIAYQGHFDYWGQGTLVTVSSGGGGSGGGGSGGG | |
| | GSGGGGSDIQMTQSPSSLSASVGDRVTITCRASQVIYSYLNWYQQKP | |
| | GKAPKLLIYAASSLKSGVPSRFSGSGSGTDFTLTISSLQPEDFATYYCQ | |
| | QVYDTPLTFG [symbol] GTKVEIK [SEQ ID NO: 221] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGGTGATCT | |
| sequence | ACAGCTACCTGAACTGGTACCAGCAGAAGCCCGGCAAGGCCCCC | |
| of Ab9 | AAGCTGCTGATCTACGCCGCCAGCAGCCTGAAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGTGTACGACACCCCCCTGACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCCGGCAAGTGCCTGGAGTGGGTGGCCAAC | |
| | ATCAACACCGACGGCAGCGAGGTGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGACGTGGGCCCCGGCATCGCCTACCAGGGCCAC | |
| | TTCGACTACTGGGGCCAGGGCACCCTGGTGACCGTGAGCAGC | |
| | [SEQ ID NO: 262] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAACACCGACGGCAGCGAGGTGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGACGTGGGCCCCGGC | |
| | ATCGCCTACCAGGGCCACTTCGACTACTGGGGCCAGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCCAGATGA | |
| | CCCAGAGCCCCAGCAGCCTGAGCGCCAGCGTGGGCGACAGGGTG | |
| | ACCATCACCTGCAGGGCCAGCCAGGTGATCTACAGCTACCTGAAC | |
| | TGGTACCAGCAGAAGCCCGGCAAGGCCCCCAAGCTGCTGATCTAC | |
| | GCCGCCAGCAGCCTGAAGAGCGGCGTGCCCAGCAGGTTCAGCGG | |
| | CAGCGGCAGCGGCACCGACTTCACCCTGACCATCAGCAGCCTGCA | |
| | GCCCGAGGACTTCGCCACCTACTACTGCCAGCAGGTGTACGACAC | |
| | CCCCCTGACCTTCGGCTGCGGCACCAAGGTGGAGATCAAG [SEQ | |
| | ID NO: 263] | |
| ADI-11835 | QLQLQESGPGLVKPSETLSLTCT | EIVLTQSPATLSLSPGERATLSCR |
| [Ab10] | VSGGSISSTDYYWGWIRQPPGK | ASHSVYSYLAWYQQKPGQAPRL |
| (I35) | GLEWIGSIGYSGTYYNPSLKSRV | LIYDASNRATGIPARFSGSGSGTD |
| | TISVDTSKNQFSLKLSSVTAADT | FTLTISSLEPEDFAVYYCQQYDN |
| | AVYYCARETAHDVHGMDVWG | LPTFGGGTKVEIK [SEQ ID |
| | QGTTVTVSS [SEQ ID NO: 19] | NO: 20] |
| | CDR1: GSISSTDYYWG [SEQ ID | CDR1: RASHSVYSYLA [SEQ ID |
| | NO: 75](non-Kabat) or STDYYWG | NO: 78] |
| | [SEQ ID NO: 445] | CDR2: DASNRAT [SEQ ID NO: 79] |
| | CDR2: SIGYSGTYYNPSLKS | CDR3: QQYDNLPT [SEQ ID |
| | [SEQ ID NO: 76] | NO: 80] |
| | CDR3: ARETAHDVHGMDV [SEQ | |
| | ID NO: 77](non-Kabat) or | |
| | ETAHDVHGMDV [SEQ ID | |
| | NO: 446] | |
| scFv of | EIVLTQSPATLSLSPGERATLSCRASHSVYSYLAWYQQKPGQAPRLLI | |
| Ab10 | YDASNRATGIPARFSGSGSGTDFTLTISSLEPEDFAVYYCQQYDNLPT | |
| | FG [symbol] GTKVEIKGGGGSGGGGSGGGGSGGGGSQLQLQESGPGLVKPS | |
| | ETLSLTCTVSGGSISSTDYYWGWIRQPPGK [symbol] LEWIGSIGYSGTYYNPS | |
| | LKSRVTISVDTSKNQFSLKLSSVTAADTAVYYCARETAHDVHGMDV | |
| | WGQGTTVTVSS [SEQ ID NO: 222] | |
| | QLQLQESGPGLVKPSETLSLTCTVSGGSISSTDYYWGWIRQPPGK [symbol] LE | |
| | WIGSIGYSGTYYNPSLKSRVTISVDTSKNQFSLKLSSVTAADTAVYYC | |
| | ARETAHDVHGMDVWGQGTTVTVSSGGGGSGGGGSGGGGSGGGG | |
| | SEIVLTQSPATLSLSPGERATLSCRASHSVYSYLAWYQQKPGQAPRLL | |
| | IYDASNRATGIPARFSGSGSGTDFTLTISSLEPEDFAVYYCQQYDNLPT | |
| | FG [symbol] GTKVEIK [SEQ ID NO: 223] | |
| Exemplary | GAGATCGTGCTGACCCAGAGCCCCGCCACCCTGAGCCTGAGCCCC | |
| nucleotide | GGCGAGAGGGCCACCCTGAGCTGCAGGGCCAGCCACAGCGTGTA | |
| sequence | CAGCTACCTGGCCTGGTACCAGCAGAAGCCCGGCCAGGCCCCCA | |
| of Ab10 | GGCTGCTGATCTACGACGCCAGCAACAGGGCCACCGGCATCCCCG | |
| scFv | CCAGGTTCAGCGGCAGCGGCAGCGGCACCGACTTCACCCTGACCA | |
| | TCAGCAGCCTGGAGCCCGAGGACTTCGCCGTGTACTACTGCCAGO | |
| | AGTACGACAACCTGCCCACCTTCGGCTGCGGCACCAAGGTGGAG | |
| | ATCAAGGGCGGCGGCGGCAGCGGCGGCGGCGGCAGCGGCGGCGG | |
| | CGGCAGCGGCGGCGGCGGCAGCCAGCTGCAGCTGCAGGAGAGCG | |
| | GCCCCGGCCTGGTGAAGCCCAGCGAGACCCTGAGCCTGACCTGCA | |
| | CCGTGAGCGGCGGCAGCATCAGCAGCACCGACTACTACTGGGGC | |
| | TGGATCAGGCAGCCCCCCGGCAAGTGCCTGGAGTGGATCGGCAG | |
| | CATCGGCTACAGCGGCACCTACTACAACCCCAGCCTGAAGAGCA | |
| | GGGTGACCATCAGCGTGGACACCAGCAAGAACCAGTTCAGCCTG | |
| | AAGCTGAGCAGCGTGACCGCCGCCGACACCGCCGTGTACTACTGC | |
| | GCCAGGGAGACCGCCCACGACGTGCACGGCATGGACGTGTGGGG | |
| | CCAGGGCACCACCGTGACCGTGAGCAGC [SEQ ID NO: 264] | |
| | CAGCTGCAGCTGCAGGAGAGCGGCCCCGGCCTGGTGAAGCCCAG | |
| | CGAGACCCTGAGCCTGACCTGCACCGTGAGCGGCGGCAGCATCA | |
| | GCAGCACCGACTACTACTGGGGCTGGATCAGGCAGCCCCCCGGC | |
| | AAGTGCCTGGAGTGGATCGGCAGCATCGGCTACAGCGGCACCTA | |
| | CTACAACCCCAGCCTGAAGAGCAGGGTGACCATCAGCGTGGACA | |
| | CCAGCAAGAACCAGTTCAGCCTGAAGCTGAGCAGCGTGACCGCC | |
| | GCCGACACCGCCGTGTACTACTGCGCCAGGGAGACCGCCCACGA | |
| | CGTGCACGGCATGGACGTGTGGGGCCAGGGCACCACCGTGACCG | |
| | TGAGCAGCGGCGGCGGCGGCAGCGGCGGCGGCGGCAGGGGCGGC | |
| | GGCGGCAGCGGCGGCGGCGGCAGCGAGATCGTGCTGACCCAGAG | |
| | CCCCGCCACCCTGAGCCTGAGCCCCGGCGAGAGGGCCACCCTGA | |
| | GCTGCAGGGCCAGCCACAGCGTGTACAGCTACCTGGCCTGGTACC | |
| | AGCAGAAGCCCGGCCAGGCCCCCAGGCTGCTGATCTACGACGCC | |
| | AGCAACAGGGCCACCGGCATCCCCGCCAGGTTCAGCGGCAGCGG | |
| | CAGCGGCACCGACTTCACCCTGACCATCAGCAGCCTGGAGCCCGA | |
| | GGACTTCGCCGTGTACTACTGCCAGCAGTACGACAACCTGCCCAC | |
| | CTTCGGCTGCGGCACCAAGGIGGAGATCAAG [SEQ ID NO: 265] | |
| ADI-10154 | EVQLLESGGGLVQPGGSLRLSC | EIVLTQSPGTLSLSPGERATLSCR |
| [Ab11] | AASGFTFSSYAMSWVRQAPGK | ASQSVSSSFLAWYQQKPGQAPR |
| | GLEWVSAISASGGSTYYADSVK | LLIYGASSRATGIPDRFSGSGSGT |
| | GRFTISRDNSKNTLYLQMNSLR | DFTLTISRLEPEDFAVYYCQQASS |
| | AEDTAVYYCARPRAYYDSSGFK | SPPTFGGGTKVEIK [SEQ ID |
| | VNYGMDVWGQGTTVTVSS | NO: 267] |
| | [SEQ ID NO: 266] | CDR1: RASQSVSSSFLA [SEQ ID |
| | CDR1: SYAMS [SEQ ID NO: 304] | NO: 307] |
| | or FTFSSYAMS [SEQ ID NO: 528] | CDR2: GASSRAT [SEQ ID NO: 308] |
| | (non-Kabat) | CDR3: QQASSSPPT [SEQ ID |
| | CDR2: AISASGGSTYYADSVKG | NO: 309] |
| | [SEQ ID NO: 305] | |
| | CDR3: | |
| | PRAYYDSSGF KVNYGMD V | |
| | [SEQ ID NO: 306] or | |
| | ARPRAYYDSSGF KVNYGMD V | |
| | [SEQ ID NO: 529](non-Kabat) | |
| scFv of | EIVLTQSPGTLSLSPGERATLSCRASQSVSSSFLAWYQQKPGQAPRLLI | |
| Ab11 | YGASSRATGIPDRFSGSGSGTDFTLTISRLEPEDFAVYYCQQASSSPPT | |
| | FG [symbol] GTKVEIKGGGGSGGGGSGGGGSGGGGSEVQLLESGGGLVQP | |
| | GGSLRLSCAASGFTFSSYAMSWVRQAPGK [symbol] LEWVSAISASGGSTYY | |
| | ADSVKGRFTISRDNSKNTLYLQMNSLRAEDTAVYYCARPRAYYDSS | |
| | GFKVNYGMDVWGQGTTVTVSS [SEQ ID NO: 447] | |
| | EVQLLESGGGLVQPGGSLRLSCAASGFTFSSYAMSWVRQAPGK [symbol] LE | |
| | WVSAISASGGSTYYADSVKGRFTISRDNSKNTLYLQMNSLRAEDTA | |
| | VYYCARPRAYYDSSGFKVNYGMDVWGQGTTVTVSSGGGGSGGGG | |
| | SGGGGSGGGGSEIVLTQSPGTLSLSPGERATLSCRASQSVSSSFLAW | |
| | YQQKPGQAPRLLIYGASSRATGIPDRFSGSGSGTDFTLTISRLEPEDFA | |
| | VYYCQQASSSPPTFG [symbol] GTKVEIK [SEQ ID NO: 448] | |
| Exemplary | GAGATCGTGCTGACCCAGAGCCCCGGCACCCTGAGCCTGAGCCCC | |
| nucleotide | GGCGAGAGGGCCACCCTGAGCTGCAGCGGCCAGCCAGAGCGTGAG | |
| sequence | CAGCAGCTTCCTGGCCTGGTACCAGCAGAAGCCCGGCCAGGCCCC | |
| of Ab11 | CAGGCTGCTGATCTACGGCGCCAGCAGCAGGGCCACCGGCATCCC | |
| scFv | CGACAGGTTCAGCGGCAGCGGCAGCGGCACCGACTTCACCCTGA | |
| | CCATCAGCAGGCTGGAGCCCGAGGACTTCGCCGTGTACTACTGCC | |
| | AGCAGGCCAGCAGCAGCCCCCCCACCTTCGGCTGCGGCACCAAG | |
| | GTGGAGATCAAGGGCGGCGGCGGCAGCGGCGGCGGCGGCAGCGG | |
| | CGGCGGCGGCAGCGGCGGCGGCGGCAGCGAGGTGCAGCTGCTGG | |
| | AGAGCGGCGGCGGCCTGGTGCAGCCCGGCGGCAGCCTGAGGCTG | |
| | AGCTGCGCCGCCAGCGGCTTCACCTTCAGCAGCTACGCCATGAGC | |
| | TGGGTGAGGCAGGCCCCCGGCAAGTGCCTGGAGTGGGTGAGCGC | |
| | CATCAGCGCCAGCGGCGGCAGCACCTACTACGCCGACAGCGTGA | |
| | AGGGCAGGTTCACCATCAGCAGGGACAACAGCAAGAACACCCTG | |
| | TACCTGCAGATGAACAGCCTGAGGGCCGAGGACACCGCCGTGTA | |
| | CTACTGCGCCAGGCCCAGGGCCTACTACGACAGCAGCGGCTTCAA | |
| | GGTGAACTACGGCATGGACGTGTGGGGCCAGGGCACCACCGTGA | |
| | CCGTGAGCAGC [SEQ ID NO: 489] | |
| | GAGGTGCAGCTGCTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACGCCATGAGCTGGGTGAGGCAGGCCCCCGGCAAGTGCC | |
| | TGGAGTGGGTGAGCGCCATCAGCGCCAGCGGCGGCAGCACCTAC | |
| | TACGCCGACAGCGTGAAGGGCAGGTTCACCATCAGCAGGGACAA | |
| | CAGCAAGAACACCCTGTACCTGCAGATGAACAGCCTGAGGGCCG | |
| | AGGACACCGCCGTGTACTACTGCGCCAGGCCCAGGGCCTACTACG | |
| | ACAGCAGCGGCTTCAAGGTGAACTACGGCATGGACGTGTGGGGC | |
| | CAGGGCACCACCGTGACCGTGAGCAGCGGCGGCGGCGGCAGCGG | |
| | CGGCGGCGGCAGCGGCGGCGGCGGCAGCGGCGGCGGCGGCAGCG | |
| | AGATCGTGCTGACCCAGAGCCCCGGCACCCTGAGCCTGAGCCCCG | |
| | GCGAGAGGGCCACCCTGAGCTGCAGGGCCAGCCAGAGCGTGAGC | |
| | AGCAGCTTCCTGGCCTGGTACCAGCAGAAGCCCGGCCAGGCCCCC | |
| | AGGCTGCTGATCTACGGCGCCAGCAGCAGGGCCACCGGCATCCCC | |
| | GACAGGTTCAGCGGCAGCGGCAGCGGCACCGACTTCACCCTGAC | |
| | CATCAGCAGGCTGGAGCCCGAGGACTTCGCCGTGTACTACTGCCA | |
| | GCAGGCCAGCAGCAGCCCCCCCACCTTCGGCTGCGGCACCAAGGT | |
| | GGAGATCAAG [SEQ ID NQ:490] | |
| ADI-10155 | EVQLLESGGGLVQPGGSLRLSC | EIVLTQSPGTLSLSPGERATLSCR |
| [Ab12] | AASGFTFSSYAMSWVRQAPGK | ASQSVSSDYLAWYQQKPGQAPR |
| | GLEWVSGISGSGGSTYYADSVK | LLIYGASSRATGIPDRFSGSGSGT |
| | GRFTISRDNSKNTLYLQMNSLR | DFTLTISRLEPEDFAVYYCQQHSS |
| | AEDTAVYYCAREGHSSSYYDH | APPTFGGGTKVEIK [SEQ ID |
| | AFDIWGQGTMVTVSS [SEQ ID | NO: 269] |
| | NO: 268] | CDR1: RASQSVSSDYLA [SEQ ID |
| | CDR1: SYAMS [SEQ ID NO: 310] | NO: 313] |
| | or FTFSSYAMS [SEQ ID NO: 530] | CDR2: GASSRAT [SEQ ID NO: 314] |
| | (non-Kabat) | CDR3: QQHSSAPPT [SEQ ID |
| | CDR2: GISGSGGSTYYADSVKG | NO: 315] |
| | [SEQ ID NO: 311] | |
| | CDR3: EGHSS SYYDHAFDI [SEQ | |
| | ID NO: 312] or | |
| | AREGHSS SYYDHAFDI [SEQ ID | |
| | NO: 531](non-Kabat) | |
| scFv of | EIVLTQSPGTLSLSPGERATLSCRASQSVSSDYLAWYQQKPGQAPRLL | |
| Ab12 | IYGASSRATGIPDRFSGSGSGTDFTLTISRLEPEDFAVYYCQQHSSAPP | |
| | TFG [symbol] GTKVEIKGGGGSGGGGSGGGGSGGGGSEVQLLESGGGLVQP | |
| | GGSLRLSCAASGFTFSSYAMSWVRQAPGK [symbol] LEWVSGISGSGGSTYY | |
| | ADSVKGRFTISRDNSKNTLYLQMNSLRAEDTAVYYCAREGHSSSYY | |
| | DHAFDIWGQGTMVTVSS [SEQ ID NO: 449] | |
| | EVQLLESGGGLVQPGGSLRLSCAASGFTFSSYAMSWVRQAPGK [symbol] LE | |
| | WVSGISGSGGSTYYADSVKGRFTISRDNSKNTLYLQMNSLRAEDTA | |
| | VYYCAREGHSSSYYDHAFDIWGQGTMVTVSSGGGGSGGGGSGGG | |
| | GSGGGGSEIVLTQSPGTLSLSPGERATLSCRASQSVSSDYLAWYQQK | |
| | PGQAPRLLIYGASSRATGIPDRFSGSGSGTDFTLTISRLEPEDFAVYYC | |
| | QQHSSAPPTFG [symbol] GTKVEIK [SEQ ID NO: 450] | |
| Exemplary | GAGATCGTGCTGACCCAGAGCCCCGGCACCCTGAGCCTGAGCCCC | |
| nucleotide | GGCGAGAGGGCCACCCTGAGCTGCAGGGCCAGCCAGAGCGTGAG | |
| sequence | CAGCGACTACCTGGCCTGGTACCAGCAGAAGCCCGGCCAGGCCC | |
| of Ab12 | CCAGGCTGCTGATCTACGGCGCCAGCAGCAGGGCCACCGGCATCC | |
| scFv | CCGACAGGTTCAGCGGCAGCGGCAGCGGCACCGACTTCACCCTG | |
| | ACCATCAGCAGGCTGGAGCCCGAGGACTTCGCCGTGTACTACTGC | |
| | CAGCAGCACAGCAGCGCCCCCCCCACCTTCGGCTGCGGCACCAAG | |
| | GTGGAGATCAAGGGCGGCGGCGGCAGCGGCGGCGGCGGCAGCGG | |
| | CGGCGGCGGCAGCGGCGGCGGCGGCAGCGAGGTGCAGCTGCTGG | |
| | AGAGCGGCGGCGGCCTGGTGCAGCCCGGCGGCAGCCTGAGGCTG | |
| | AGCTGCGCCGCCAGCGGCTTCACCTTCAGCAGCTACGCCATGAGC | |
| | TGGGTGAGGCAGGCCCCCGGCAAGTGCCTGGAGTGGGTGAGCGG | |
| | CATCAGCGGCAGCGGCGGCAGCACCTACTACGCCGACAGCGTGA | |
| | AGGGCAGGTTCACCATCAGCAGGGACAACAGCAAGAACACCCTG | |
| | TACCTGCAGATGAACAGCCTGAGGGCCGAGGACACCGCCGTGTA | |
| | CTACTGCGCCAGGGAGGGCCACAGCAGCAGCTACTACGACCACG | |
| | CCTTCGACATCTGGGGCCAGGGCACCATGGTGACCGTGAGCAGC | |
| | [SEQ ID NO: 491] | |
| | GAGGTGCAGCTGCTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACGCCATGAGCTGGGTGAGGCAGGCCCCCGGCAAGTGCC | |
| | TGGAGTGGGTGAGCGGCATCAGCGGCAGCGGCGGCAGCACCTAC | |
| | TACGCCGACAGCGTGAAGGGCAGGTTCACCATCAGCAGGGACAA | |
| | CAGCAAGAACACCCTGTACCTGCAGATGAACAGCCTGAGGGCCG | |
| | AGGACACCGCCGTGTACTACTGCGCCAGGGAGGGCCACAGCAGC | |
| | AGCTACTACGACCACGCCTTCGACATCTGGGGCCAGGGCACCATG | |
| | GTGACCGTGAGCAGCGGCGGCGGCGGCAGCGGCGGCGGCGGCAG | |
| | CGGCGGCGGCGGCAGCGGCGGCGGCGGCAGCGAGATCGTGCTGA | |
| | CCCAGAGCCCCGGCACCCTGAGCCTGAGCCCCGGCGAGAGGGCC | |
| | ACCCTGAGCTGCAGGGCCAGCCAGAGCGTGAGCAGCGACTACCT | |
| | GGCCTGGTACCAGCAGAAGCCCGGCCAGGCCCCCAGGCTGCTGA | |
| | TCTACGGCGCCAGCAGCAGGGCCACCGGCATCCCCGACAGGTTCA | |
| | GCGGCAGCGGCAGCGGCACCGACTTCACCCTGACCATCAGCAGG | |
| | CTGGAGCCCGAGGACTTCGCCGTGTACTACTGCCAGCAGCACAGC | |
| | AGCGCCCCCCCCACCTTCGGCTGCGGCACCAAGGTGGAGATCAAG | |
| | [SEQ ID NO: 492] | |
| ADI-10157 | QVQLQESGPGLVKPSETLSLTCT | EIVMTQSPATLSVSPGERATLSCR |
| [Ab13] | VSGGSISSYYWSWIRQPPGKGLE | ASQSVSSNLAWYQQKPGQAPRL |
| | WIGSIYYSGSTNYNPSLKSRVTIS | LIYGASTRATGIPARFSGSGSGTE |
| | VDTSKNQFSLKLSSVTAADTAV | FTLTISSLQSEDFAVYYCQQYTV |
| | YYCARVGGVYSTIETYGMDVW | YPPTFGGGTKVEIK [SEQ ID |
| | GQGTTVTVSS [SEQ ID NO: 270] | NO: 271] |
| | CDR1: SYYWS [SEQ ID NO: 316] | CDR1: RASQSVSSNLA [SEQ ID |
| | or GSISSYYWS [SEQ ID NO: 532] | NO: 319] |
| | (non-Kabat) | CDR2: GASTRAT [SEQ ID |
| | CDR2: SIYYSGSTNYNPSLKS | NO: 320] |
| | [SEQ ID NO: 317] | CDR3: QQYTVYPPT [SEQ ID |
| | CDR3: VGGVYSTIETYGMDV | NO: 321] |
| | [SEQ ID NO: 318] or | |
| | ARVGGVYSTIETYGMDV [SEQ | |
| | ID NO: 533](non-Kabat) | |
| scFv of | EIVMTQSPATLSVSPGERATLSCRASQSVSSNLAWYQQKPGQAPRLLI | |
| Ab13 | YGASTRATGIPARFSGSGSGTEFTLTISSLQSEDFAVYYCQQYTVYPP | |
| | TFG [symbol] GTKVEIKGGGGSGGGGSGGGGSGGGGSQVQLQESGPGLVKP | |
| | SETLSLTCTVSGGSISSYYWSWIRQPPGK [symbol] LEWIGSIYYSGSTNYNPSL | |
| | KSRVTISVDTSKNQFSLKLSSVTAADTAVYYCARVGGVYSTIETYGM | |
| | DVWGQGTTVTVSS [SEQ ID NO: 451] | |
| | QVQLQESGPGLVKPSETLSLTCTVSGGSISSYYWSWIRQPPGK [symbol] LEWI | |
| | GSIYYSGSTNYNPSLKSRVTISVDTSKNQFSLKLSSVTAADTAVYYCA | |
| | RVGGVYSTIETYGMDVWGQGTTVTVSSGGGGSGGGGSGGGGSGG | |
| | GGSEIVMTQSPATLSVSPGERATLSCRASQSVSSNLAWYQQKPGQAP | |
| | RLLIYGASTRATGIPARFSGSGSGTEFTLTISSLQSEDFAVYYCQQYTV | |
| | YPPTFG [symbol] GTKVEIK [SEQ ID NO: 452] | |
| Exemplary | GAGATCGTGATGACCCAGAGCCCCGCCACCCTGAGCGTGAGCCCC | |
| nucleotide | GGCGAGAGGGCCACCCTGAGCTGCAGGGCCAGCCAGAGCGTGAG | |
| sequence | CAGCAACCTGGCCTGGTACCAGCAGAAGCCCGGCCAGGCCCCCA | |
| of Ab13 | GGCTGCTGATCTACGGCGCCAGCACCAGGGCCACCGGCATCCCCG | |
| scFv | CCAGGTTCAGCGGCAGCGGCAGCGGCACCGAGTTCACCCTGACC | |
| | ATCAGCAGCCTGCAGAGCGAGGACTTCGCCGTGTACTACTGCCAG | |
| | CAGTACACCGTGTACCCCCCCACCTTCGGCTGCGGCACCAAGGTG | |
| | GAGATCAAGGGCGGCGGCGGCAGCGGCGGCGGCGGCAGCGGCG | |
| | GCGGCGGCAGCGGCGGCGGCGGCAGCCAGGTGCAGCTGCAGGAG | |
| | AGCGGCCCCGGCCTGGTGAAGCCCAGCGAGACCCTGAGCCTGAC | |
| | CTGCACCGTGAGCGGCGGCAGCATCAGCAGCTACTACTGGAGCTG | |
| | GATCAGGCAGCCCCCCGGCAACTGCCTGGAGTGGATCGGCAGCA | |
| | TCTACTACAGCGGCAGCACCAACTACAACCCCAGCCTGAAGAGC | |
| | AGGGTGACCATCAGCGTGGACACCAGCAAGAACCAGTTCAGCCT | |
| | GAAGCTGAGCAGCGTGACCGCCGCCGACACCGCCGTGTACTACTG | |
| | CGCCAGGGTGGGCGGCGTGTACAGCACCATCGAGACCTACGGCA | |
| | TGGACGTGTGGGGCCAGGGCACCACCGTGACCGTGAGCAGC [SEQ | |
| | ID NO: 493] | |
| | CAGGTGCAGCTGCAGGAGAGCGGCCCCGGCCTGGTGAAGCCCAG | |
| | CGAGACCCTGAGCCTGACCTGCACCGTGAGCGGCGGCAGCATCA | |
| | GCAGCTACTACTGGAGCTGGATCAGGCAGCCCCCCGGCAAGTGCC | |
| | TGGAGTGGATCGGCAGCATCTACTACAGCGGCAGCACCAACTAC | |
| | AACCCCAGCCTGAAGAGCAGGGTGACCATCAGCGTGGACACCAG | |
| | CAAGAACCAGTTCAGCCTGAAGCTGAGCAGCGTGACCGCCGCCG | |
| | ACACCGCCGTGTACTACTGCGCCAGGGTGGGCGGCGTGTACAGCA | |
| | CCATCGAGACCTACGGCATGGACGTGTGGGGCCAGGGCACCACC | |
| | GTGACCGTGAGCAGCGGCGGCGGCGGCAGCGGCGGCGGCGGCAG | |
| | CGGCGGCGGCGGCAGCGGCGGCGGCGGCAGCGAGATCGTGATGA | |
| | CCCAGAGCCCCGCCACCCTGAGCGTGAGCCCCGGCGAGAGGGCC | |
| | ACCCTGAGCTGCAGGGCCAGCCAGAGCGTGAGCAGCAACCTGGC | |
| | CTGGTACCAGCAGAAGCCCGGCCAGGCCCCCAGGCTGCTGATCTA | |
| | CGGCGCCAGCACCAGGGCCACCGGCATCCCCGCCAGGTTCAGOG | |
| | GCAGCGGGAGCGGCACCGAGTTCACCCTGACCATCAGCAGCCTGC | |
| | AGAGCGAGGACTTCGCCGTGTACTACTGCCAGCAGTACACCGTGT | |
| | ACCCCCCCACCTTCGGCTGCGGCACCAAGGTGGAGATCAAG [SEQ | |
| | ID NO: 494] | |
| ADI-10158 | QVQLQQWGAGLLKPSETLSLTC | EIVMTQSPATLSLSPGERATLSCR |
| [Ab14] | AVYGGSFSGYYWSWIRQPPGK | ASQSVSSYLAWYQQKPGQAPRL |
| | GLEWIGEIDHSGSTNYNPSLKSR | LIYDASNRATGIPARFSGSGSGTD |
| | VTISVDTSKNQFSLKLSSVTAAD | FTLTISSLEPEDFAVYYCQQDHNF |
| | TAVYYCARQGIHGLRYFDLWG | PYTFGGGTKVEIK [SEQ ID |
| | RGTLVTVSS [SEQ ID NO: 272] | NO: 273] |
| | CDR1: GYYWS [SEQ ID NO: 322] | CDR1: RASQSVSSYLA [SEQ ID |
| | or GSFSGYYWS [SEQ ID NO: 534] | NO: 325] |
| | (non-Kabat) | CDR2: DASNRAT [SEQ ID |
| | CDR2: EIDHSGSTNYNPSLKS | NO: 326] |
| | [SEQ ID NO: 323] | CDR3: QQDHNFPYT [SEQ ID |
| | CDR3: QGIHGLRYFDL [SEQ ID | NO: 327] |
| | NO: 324] or ARQGIHGLRYFDL | |
| | [SEQ ID NO: 535](non-Kabat) | |
| scFv of | EIVMTQSPATLSLSPGERATLSCRASQSVSSYLAWYQQKPGQAPRLLI | |
| Ab14 | YDASNRATGIPARFSGSGSGTDFTLTISSLEPEDFAVYYCQQDHNFPY | |
| | TFG [symbol] GTKVEIKGGGGSGGGGSGGGGSGGGGSQVQLQQWGAGLLK | |
| | PSETLSLTCAVYGGSFSGYYWSWIRQPPGK [symbol] LEWIGEIDHSGSTNYN | |
| | PSLKSRVTISVDTSKNQFSLKLSSVTAADTAVYYCARQGIHGLRYFD | |
| | LWGRGTLVTVSS [SEQ ID NO: 453] | |
| | QVQLQQWGAGLLKPSETLSLTCAVYGGSFSGYYWSWIRQPPGK [symbol] LE | |
| | WIGEIDHSGSTNYNPSLKSRVTISVDTSKNQFSLKLSSVTAADTAVYY | |
| | CARQGIHGLRYFDLWGRGTLVTVSSGGGGSGGGGSGGGGSGGGG | |
| | SEIVMTQSPATLSLSPGERATLSCRASQSVSSYLAWYQQKPGQAPRL | |
| | LIYDASNRATGIPARFSGSGSGTDFTLTISSLEPEDFAVYYCQQDHNFP | |
| | YTFG [symbol] GTKVEIK [SEQ ID NO: 454] | |
| Exemplary | GAGATCGTGATGACCCAGAGCCCCGCCACCCTGAGCCTGAGCCCC | |
| nucleotide | GGCGAGAGGGCCACCCTGAGCTGCAGGGCCAGCCAGAGCGTGAG | |
| sequence | CAGCTACCTGGCCTGGTACCAGCAGAAGCCCGGCCAGGCCCCCA | |
| of Ab14 | GGCTGCTGATCTACGACGCCAGCAACAGGGCCACCGGCATCCCCG | |
| scFv | CCAGGTTCAGCGGCAGCGGCAGCGGCACCGACTTCACCCTGACCA | |
| | TCAGCAGCCTGGAGCCCGAGGACTTCGCCGTGTACTACTGCCAGC | |
| | AGGACCACAACTTCCCCTACACCTTCGGCTGCGGCACCAAGGTGG | |
| | AGATCAAGGGCGGCGGCGGCAGCGGCGGCGGCGGCAGCGGCGGC | |
| | GGCGGCAGCGGCGGCGGCGGCAGCCAGGTGCAGCTGCAGCAGTG | |
| | GGGCGCCGGCCTGCTGAAGCCCAGCGAGACCCTGAGCCTGACCT | |
| | GCGCCGTGTACGGCGGCAGCTTCAGCGGCTACTACTGGAGCTGGA | |
| | TCAGGCAGCCCCCCGGCAAGTGCCTGGAGTGGATCGGCGAGATC | |
| | GACCACAGCGGCAGCACCAACTACAACCCCAGCCTGAAGAGCAG | |
| | GGTGACCATCAGCGTGGACACCAGCAAGAACCAGTTCAGCCTGA | |
| | AGCTGAGCAGCGTGACCGCCGCCGACACCGCCGTGTACTACTGCG | |
| | CCAGGCAGGGCATCCACGGCCTGAGGTACTTCGACCTGTGGGGCA | |
| | GGGGCACCCTGGTGACCGTGAGCAGC [SEQ ID NO: 495] | |
| | CAGGTGCAGCTGCAGCAGTGGGGCGCCGGCCTGCTGAAGCCCAG | |
| | CGAGACCCTGAGCCTGACCTGCGCCGTGTACGGCGGCAGCTTCAG | |
| | CGGCTACTACTGGAGCTGGATCAGGCAGCCCCCCGGCAAGTGCCT | |
| | GGAGTGGATCGGCGAGATCGACCACAGCGGCAGCACCAACTACA | |
| | ACCCCAGCCTGAAGAGCAGGGTGACCATCAGCGTGGACACCAGC | |
| | AAGAACCAGTTCAGCCTGAAGCTGAGCAGCGTGACCGCCGCCGA | |
| | CACCGCCGTGTACTACTGCGCCAGGCAGGGCATCCACGGCCTGAG | |
| | GTACTTCGACCTGTGGGGCAGGGGCACCCTGGTGACCGTGAGCAG | |
| | CGGCGGCGGCGGCAGCGGCGGCGGCGGCAGCGGCGGCGGCGGCA | |
| | GCGGCGGCGGCGGCAGCGAGATCGTGATGACCCAGAGCCCCGCC | |
| | ACCCTGAGCCTGAGCCCCGGCGAGAGGGCCACCCTGAGCTGCAG | |
| | GGCCAGCCAGAGCGTGAGCAGCTACCTGGCCTGGTACCAGCAGA | |
| | AGCCCGGCCAGGCCCCCAGGCTGCTGATCTACGACGCCAGCAAC | |
| | AGGGCCACCGGCATCCCCGCCAGGTTCAGCGGCAGCGGCAGCGG | |
| | CACCGACTTCACCCTGACCATCAGCAGCCTGGAGCCCGAGGACTT | |
| | CGCCGTGTACTACTGCCAGCAGGACCACAACTTCCCCTACACCTT | |
| | CGGCTGCGGCACCAAGGTGGAGATCAAG [SEQ ID NO: 496] | |
| ADI-10160 | EVQLVESGGGLVQPGGSLRLSC | DIQMTQSPSSLSASVGDRVTITCR |
| [Ab15] | AASGFTFSSYWMSWVRQAPGK | ASQSISSYLNWYQQKPGKAPKLL |
| | GLEWVANINQDGSEKYYVDSV | IYAASSLQSGVPSRFSGSGSGTDF |
| | KGRFTISRDNAKNSLYLQMNSL | TLTISSLQPEDFATYYCQQQYVT |
| | RAEDTAVYYCAREANYYGNVG | PITFGGGTKVEIK [SEQ ID |
| | DDYWGQGTLVTVSS [SEQ ID | NO: 275] |
| | NO: 274] | CDR1: RASQSISSYLN [SEQ ID |
| | CDR1: SYWMS [SEQ ID NO: 328] | NO: 331] |
| | or FTFSSYWMS [SEQ ID NO: 536] | CDR2: AASSLQS [SEQ ID NO: 332] |
| | (non-Kabat) | CDR3: QQQYVTPIT [SEQ ID |
| | CDR2: NINQDGSEKYYVDSVKG | NO: 333] |
| | [SEQ ID NO: 329] | |
| | CDR3: EANYYGNVGDDY [SEQ | |
| | ID NO: 330] or | |
| | AREANYYGNVGDDY [SEQ ID | |
| | NO: 537](non-Kabat) | |
| scFv of | DIQMTQSPSSLSASVGDRVTITCRASQSISSYLNWYQQKPGKAPKLLI | |
| Ab15 | YAASSLQSGVPSRFSGSGSGTDFTLTISSLQPEDFATYYCQQQYVTPIT | |
| | FG [symbol] GTKVEIKGGGGSGGGGSGGGGSGGGGSEVQLVESGGGLVQP | |
| | GGSLRLSCAASGFTFSSYWMSWVRQAPGK [symbol] LEWVANINQDGSEKY | |
| | YVDSVKGRFTISRDNAKNSLYLQMNSLRAEDTAVYYCAREANYYG | |
| | NVGDDYWGQGTLVTVSS [SEQ ID NO: 455] | |
| | EVQLVESGGGLVQPGGSLRLSCAASGFTFSSYWMSWVRQAPGK [symbol] LE | |
| | WVANINQDGSEKYYVDSVKGRFTISRDNAKNSLYLQMNSLRAEDTA | |
| | VYYCAREANYYGNVGDDYWGQGTLVTVSSGGGGSGGGGSGGGG | |
| | SGGGGSDIQMTQSPSSLSASVGDRVTITCRASQSISSYLNWYQQKPG | |
| | KAPKLLIYAASSLQSGVPSRFSGSGSGTDFTLTISSLQPEDFATYYCQQ | |
| | QYVTPITFG [symbol] GTKVEIK [SEQ ID NO: 456] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCA | |
| sequence | GCAGCTACCTGAACTGGTACCAGCAGAAGCCCGGCAAGGCCCCC | |
| of Ab15 | AAGCTGCTGATCTACGCCGCCAGCAGCCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGCAGTACGTGACCCCCATCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCCGGCAAGTGCCTGGAGTGGGTGGCCAAC | |
| | ATCAACCAGGACGGCAGCGAGAAGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGAGGCCAACTACTACGGCAACGTGGGCGACGA | |
| | CTACTGGGGCCAGGGCACCCTGGTGACCGTGAGCAGC [SEQ ID | |
| | NO: 497] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAACCAGGACGGCAGCGAGAAGTAC | |
| | TACGTGGACAGCGTGAAGGGCAGGITCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGCCAACTACTAC | |
| | GGCAACGTGGGCGACGACTACTGGGGCCAGGGCACCCTGGTGAC | |
| | CGTGAGCAGCGGCGGCGGCGGCAGCGGCGGCGGCGGCAGCGGCG | |
| | GCGGCGGCAGCGGCGGCGGCGGCAGCGACATCCAGATGACCCAG | |
| | AGCCCCAGCAGCCTGAGCGCCAGCGTGGGCGACAGGGTGACCAT | |
| | CACCTGCAGGGCCAGCCAGAGCATCAGCAGCTACCTGAACTGGT | |
| | ACCAGCAGAAGCCCGGCAAGGCCCCCAAGCTGCTGATCTACGCC | |
| | GCCAGCAGCCTGCAGAGCGGCGTGCCCAGCAGGTTCAGCGGCAG | |
| | CGGCAGCGGCACCGACTTCACCCTGACCATCAGCAGCCTGCAGCC | |
| | CGAGGACTTCGCCACCTACTACTGCCAGCAGCAGTACGTGACCCC | |
| | CATCACCTTCGGCTGCGGCACCAAGGTGGAGATCAAG [SEQ ID | |
| | NO: 498] | |
| ADI-10161 | EVQLVESGGGLVQPGGSLRLSC | DIQMTQSPSSVSASVGDRVTITC |
| [Ab16] | AASGFTFSSYWMSWVRQAPGK | RASQGISSWLAWYQQKPGKAPK |
| | GLEWVANINQDGSEKYYVDSV | LLIYAASNLQSGVPSRFSGSGSGT |
| | KGRFTISRDNAKNSLYLQMNSL | DFTLTISSLQPEDFATYYCQQKLS |
| | RAEDTAVYYCAREGGDSWYHA | LPLTFGGGTKVEIK [SEQ ID |
| | FDIWGQGTMVTVSS [SEQ ID | NO: 277] |
| | NO: 276] | CDR1: RASQGISSWLA [SEQ ID |
| | CDR1: SYWMS [SEQ ID NO: 334] | NO: 337] |
| | or FTFSSYWMS [SEQ ID NO: 538] | CDR2: AASNLQS [SEQ ID |
| | (non-Kabat) | NO: 338] |
| | CDR2: NINQDGSEKYYVDSVKG | CDR3: QQKLSLPLT [SEQ ID |
| | [SEQ ID NO: 335] | NO: 339] |
| | CDR3: EGGDSWYHAFDI [SEQ | |
| | ID NO: 336] or | |
| | AREGGDSWYHAFDI [SEQ ID | |
| | NO: 539](non-Kabat) | |
| scFv of | DIQMTQSPSSVSASVGDRVTITCRASQGISSWLAWYQQKPGKAPKLL | |
| Ab16 | IYAASNLQSGVPSRFSGSGSGTDFTLTISSLQPEDFATYYCQQKLSLPL | |
| | TFG [symbol] GTKVEIKGGGGSGGGGSGGGGSGGGGSEVQLVESGGGLVQ | |
| | PGGSLRLSCAASGFTFSSYWMSWVRQAPGK [symbol] LEWVANINQDGSEKY | |
| | YVDSVKGRFTISRDNAKNSLYLQMNSLRAEDTAVYYCAREGGDSW | |
| | YHAFDIWGQGTMVTVSS [SEQ ID NO: 457] | |
| | EVQLVESGGGLVQPGGSLRLSCAASGFTFSSYWMSWVRQAPGK [symbol] LE | |
| | WVANINQDGSEKYYVDSVKGRFTISRDNAKNSLYLQMNSLRAEDTA | |
| | VYYCAREGGDSWYHAFDIWGQGTMVTVSSGGGGSGGGGSGGGG | |
| | SGGGGSDIQMTQSPSSVSASVGDRVTITCRASQGISSWLAWYQQKP | |
| | GKAPKLLIYAASNLQSGVPSRFSGSGSGTDFTLTISSLQPEDFATYYC | |
| | QQKLSLPLTFG [symbol] GTKVEIK [SEQ ID NO: 458] | |
| Exemplary | GACATCCAGATGACCCAGAGCCCCAGCAGCGTGAGCGCCAGCGT | |
| nucleotide | GGGCGACAGGGTGACCATCACCTGCAGGGCCAGCCAGGGCATCA | |
| sequence | GCAGCTGGCTGGCCTGGTACCAGCAGAAGCCCGGCAAGGCCCCC | |
| of Ab16 | AAGCTGCTGATCTACGCCGCCAGCAACCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGAAGCTGAGCCTGCCCCTGACCTTCGGCTGCGGCACCAAGOT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCCGGCAAGTGCCTGGAGTGGGTGGCCAAC | |
| | ATCAACCAGGACGGCAGCGAGAAGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGAGGGCGGCGACAGCTGGTACCACGCCTTCGA | |
| | CATCTGGGGCCAGGCCACCATGGTGACCGTGAGCAGC [SEQ ID | |
| | NO: 499] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAACCAGGACGGCAGCGAGAAGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGGCGGCGACAGC | |
| | TGGTACCACGCCTTCGACATCTGGGGCCAGGGCACCATGGTGACC | |
| | GTGAGCAGCGGCGGCGGCGGCAGCGGCGGCGGCGGCAGCGGCGG | |
| | CGGCGGCAGCGGCGGCGGCGGCAGCGACATCCAGATGACCCAGA | |
| | GCCCCAGCAGCGTGAGCGCCAGCGTGGGCGACAGGGTGACCATC | |
| | ACCTGCAGGGCCAGCCAGGGCATCAGCAGCTGGCTGGCCTTGGTA | |
| | CCAGCAGAAGCCCGGCAAGGCCCCCAAGCTGCTGATCTACGCCG | |
| | CCAGCAACCTGCAGAGCGGCGTGCCCAGCAGGTTCAGCGGCAGC | |
| | GGCAGCGGCACCGACTTCACCCTGACCATCAGCAGCCTGCAGCCC | |
| | GAGGACTTCGCCACCTACTACTGCCAGCAGAAGCTGAGCCTGCCC | |
| | CTGACCTTCGGCTGCGGCACCAAGGTGGAGATCAAG [SEQ ID | |
| | NO: 500] | |
| ADI-10163 | QVQLQESGPGLVKPSQTLSLTCT | DIQMTQSPSSLSASVGDRVTITCR |
| [Ab17] | VSGGSISSGGYYWSWIRQHPGK | ASQSISSYLNWYQQKPGKAPKLL |
| | GLEWIGSIYYSGSTYYNPSLKSR | IYGASSLQSGVPSRFSGSGSGTDF |
| | VTISVDTSKNQFSLKLSSVTAAD | TLTISSLQPEDFATYYCQQVYSAP |
| | TAVYYCARDRLDYSYNYGMDV | FTFGGGTKVEIK [SEQ ID NO: 279] |
| | WGQGTTVTVSS [SEQ ID | CDR1: RASQSISSYLN [SEQ ID |
| | NO: 278] | NO: 343] |
| | CDR1: SGGYYWS [SEQ ID | CDR2: GASSLQS [SEQ ID NO: 344] |
| | NO: 340] or GSISSGGYYWS [SEQ | CDR3: QQVYSAPFT [SEQ ID |
| | ID NO: 540](non-Kabat) | NO: 345] |
| | CDR2: SIYYSGSTYYNPSLKS | |
| | [SEQ ID NO: 341] | |
| | CDR3: DRLDYSYNYGMDV [SEQ | |
| | ID NO: 342] or | |
| | ARDRLDYSYNYGMDV [SEQ ID | |
| | NO: 541](non-Kabat) | |
| scFv of | DIQMTQSPSSLSASVGDRVTITCRASQSISSYLNWYQQKPGKAPKLLI | |
| Ab17 | YGASSLQSGVPSRFSGSGSGTDFTLTISSLQPEDFATYYCQQVYSAPF | |
| | TFG [symbol] GTKVEIKGGGGSGGGGSGGGGSGGGGSQVQLQESGPGLVKP | |
| | SQTLSLTCTVSGGSISSGGYYWSWIRQHPGK [symbol] LEWIGSIYYSGSTYYN | |
| | PSLKSRVTISVDTSKNQFSLKLSSVTAADTAVYYCARDRLDYSYNYG | |
| | MDVWGQGTTVTVSS [SEQ ID NO: 459] | |
| | QVQLQESGPGLVKPSQTLSLTCTVSGGSISSGGYYWSWIRQHPGK [symbol] L | |
| | EWIGSIYYSGSTYYNPSLKSRVTISVDTSKNQFSLKLSSVTAADTAVY | |
| | YCARDRLDYSYNYGMDVWGQGTTVTVSSGGGGSGGGGSGGGGS | |
| | GGGGSDIQMTQSPSSLSASVGDRVTITCRASQSISSYLNWYQQKPGK | |
| | APKLLIYGASSLQSGVPSRFSGSGSGTDFTLTISSLQPEDFATYYCQQV | |
| | YSAPFTFG [symbol] GTKVEIK [SEQ ID NO: 460] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCA | |
| sequence | GCAGCTACCTGAACTGGTACCAGCAGAAGCCCGGCAAGGCCCCC | |
| of Ab17 | AAGCTGCTGATCTACGGCGCCAGCAGCCTGGAGAGCGGCGTGCCC | |
| scFv | AAGCGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGTGTACAGCGCCCCCTTCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCCAGGTGCAGCTGCAGGA | |
| | GAGCGGCCCCGGCCTGGTGAAGCCCAGCCAGACCCTGAGCCTGA | |
| | CCTGCACCGTGAGCGGCGGCAGCATCAGCAGCGGCGGCTACTACT | |
| | GGAGCTGGATCAGGCAGCACCCCGGCAAGTGCCTGGAGTGGATC | |
| | GGCAGCATCTACTACAGCGGCAGCACCTACTACAACCCCAGCCTG | |
| | AAGAGCAGGGTGACCATCAGCGTGGACACCAGCAAGAACCAGTT | |
| | CAGCCTGAAGCTGAGCAGCGTGACCGCCGCCGACACCGCCGTGT | |
| | ACTACTGCGCCAGGGACAGGCTGGACTACAGCTACAACTACGGC | |
| | ATGGACGTGTGGGGCCAGGGCACCACCGTGACCGTGAGCAGC | |
| | [SEQ ID NO: 501] | |
| | CAGGTGCAGCTGCAGGAGAGCGGCCCCGGCCTGGTGAAGCCCAG | |
| | CCAGACCCTGAGCCTGACCTGCACCGTGAGCGGCGGCAGCATCA | |
| | GCAGCGGCGGCTACTACTGGAGCTGGATCAGGCAGCACCCCGGC | |
| | AAGTGCCTGGAGTGGATCGGCAGCATCTACTACAGCGGCAGCAC | |
| | CTACTACAACCCCAGCCTGAAGAGCAGGGTGACCATCAGCGTGG | |
| | ACACCAGCAAGAACCAGTTCAGCCTGAAGCTGAGCAGCGTGACC | |
| | GCCGCCGACACCGCCGTGTACTACTGCGCCAGGGACAGGCTGGA | |
| | CTACAGCTACAACTACGGCATGGACGTGTGGGGCCAGGGCACCA | |
| | CCGTGACCGTGAGCAGCGGCGGCGGCGGCAGCGGCGGCGGCGGC | |
| | AGCGGCGGCGGCGGCAGCGGCGGCGGCGGCAGCGACATCCAGAT | |
| | GACCCAGAGCCCCAGCAGCCTGAGCGCCAGCGTGGGCGACAGGG | |
| | TGACCATCACCTGCAGGGCCAGCCAGAGCATCAGCAGCTACCTGA | |
| | ACTGGTACCAGCAGAAGCCCGGCAAGGCCCCCAAGCTGCTGATCT | |
| | ACGGCGCCAGCAGCCTGCAGAGCGGCGTGCCCAGCAGGTTCAGC | |
| | GGCAGCGGCAGCGGCACCGACTTCACCCTGACCATCAGCAGCCTG | |
| | CAGCCCGAGGACTTCGCCACCTACTACTGCCAGCAGGTGTACAGC | |
| | GCCCCCTTCACCTTCGGCTGCGGCACCAAGGTGGAGATCAAG | |
| | [SEQ ID NO: 502] | |
| ADI-10164 | QVQLQESGPGLVKPSETLSLTCA | EIVLTQSPATLSLSPGERATLSCR |
| [Ab18] | VSGYSISSGYYWGWIRQPPGKG | ASQSVSSYLAWYQQKPGQAPRL |
| | LEWIGSIYHSGSTNYNPSLKSRV | LIYDASNRATGIPARFSGSGSGTD |
| | TISVDTSKNQFSLKLSSVTAADT | FTLTISSLEPEDFAVYYCQQVDN |
| | AVYYCARLPPWFGFSYFDLWG | YPPTFGGGTKVEIK [SEQ ID |
| | RGTLVTVSS [SEQ ID NO: 280] | NO: 281] |
| | CDR1: SGYYWG [SEQ ID | CDR1: RASQSVSSYLA [SEQ ID |
| | NO: 346] or YSISSGYYWG [SEQ | NO: 349] |
| | ID NO: 542](non-Kabat) | CDR2: DASNRAT [SEQ ID |
| | CDR2: SIYHSGSTNYNPSLKS | NO: 350] |
| | [SEQ ID NO: 347] | CDR3: QQVDNYPPT [SEQ ID |
| | CDR3: LPPWFGFSYFDL [SEQ ID | NO: 351] |
| | NO: 348] or ARLPPWFGFSYFDL | |
| | [SEQ ID NO: 543](non-Kabat) | |
| scFv of | EIVLTQSPATLSLSPGERATLSCRASQSVSSYLAWYQQKPGQAPRLLI | |
| Ab18 | YDASNRATGIPARFSGSGSGTDFTLTISSLEPEDFAVYYCQQVDNYPP | |
| | TFG [symbol] GTKVEIKGGGGSGGGGSGGGGSGGGGSQVQLQESGPGLVKP | |
| | SETLSLTCAVSGYSISSGYYWGWIRQPPGK [symbol] LEWIGSIYHSGSTNYNP | |
| | SLKSRVTISVDTSKNQFSLKLSSVTAADTAVYYCARLPPWFGFSYFD | |
| | LWGRGTLVTVSS [SEQ ID NO: 461] | |
| | QVQLQESGPGLVKPSETLSLTCAVSGYSISSGYYWGWIRQPPGK [symbol] LE | |
| | WIGSIYHSGSTNYNPSLKSRVTISVDTSKNQFSLKLSSVTAADTAVYY | |
| | CARLPPWFGFSYFDLWGRGTLVTVSSGGGGSGGGGSGGGGSGGG | |
| | GSEIVLTQSPATLSLSPGERATLSCRASQSVSSYLAWYQQKPGQAPRL | |
| | LIYDASNRATGIPARFSGSGSGTDFTLTISSLEPEDFAVYYCQQVDNY | |
| | PPTFG [symbol] GTKVEIK [SEQ ID NO: 462] | |
| Exemplary | GAGATCGTGCTGACCCAGAGCCCCGCCACCCTGAGCCTGAGCCCC | |
| nucleotide | GGCGAGAGGGCCACCCTGAGCTGCAGGGCCAGCCAGAGCGTGAG | |
| sequence | CAGCTACCTGGCCTGGTACCAGCAGAAGCCCGGCCAGGCCCCCA | |
| of Ab18 | GGCTGCTGATCTACGACGCCAGCAACAGGGCCACCGGCATCCCCG | |
| scFv | CCAGGTTCAGCGGCAGCGGCAGCGGCACCGACTTCACCCTGACCA | |
| | TCAGCAGCCTGGAGCCCGAGGACTTCGCCGTGTACTACTGCCAGC | |
| | AGGTGGACAACTACCCCCCCACCTTCGGCTGCGGCACCAAGGTGG | |
| | AGATCAAGGGCGGCGGCGGCAGCGGCGGCGGCGGCAGCGGCGGC | |
| | GGCGGCAGCGGCGGCGGCGGCAGCCAGGTGCAGCTGCAGGAGAG | |
| | CGGCCCCGGCCTGGTGAAGCCCAGCGAGACCCTGAGCCTGACCTG | |
| | CGCCGTGAGCGGCTACAGCATCAGCAGCGGCTACTACTGGGGCTG | |
| | GATCAGGCAGCCCCCCGGCAAGTGCCTGGAGTGGATCGGCAGCA | |
| | TCTACCACAGCGGCAGCACCAACTACAACCCCAGCCTGAAGAGC | |
| | AGGGTGACCATCAGCGTGGACACCAGCAAGAACCAGTTCAGCCT | |
| | GAAGCTGAGCAGCGTGACCGCCGCCGACACCGCCGTGTACTACTG | |
| | CGCCAGGCTGCCCCCCTGGTTCGGCTTCAGCTACTTCGACCTGTG | |
| | GGGCAGGGGCACCCTGGTGACCGTGAGCAGC [SEQ ID NO: 503] | |
| | CAGGTGCAGCTGCAGGAGAGCGGCCCCGGCCTGGTGAAGCCCAG | |
| | CGAGACCCTGAGCCTGACCTGCGCCGTGAGCGGCTACAGCATCAG | |
| | CAGCGGCTACTACTGGGGCTGGATCAGGCAGCCCCCCGGCAAGT | |
| | GCCTGGAGTGGATCGGCAGCATCTACCACAGCGGCAGCACCAAC | |
| | TACAACCCCAGCCTGAAGAGCAGGGTGACCATCAGCGTGGACAC | |
| | CAGCAAGAACCAGTTCAGCCTGAAGCTGAGCAGCGTGACCGCCG | |
| | CCGACACCGCCGTGTACTACTGCGCCAGGCTGCCCCCCTGGTTCG | |
| | GCTTCAGCTACTTCGACCTGTGGGGCAGGGGCACCCTGGTGACCG | |
| | TGAGCAGCGGCGGCGGCGGCAGCGGCGGCGGCGGCAGCGGCGGC | |
| | GGCGGCAGCGGCGGCGGCGGCAGCGAGATCGTGCTGACCCAGAG | |
| | CCCCGCCACCCTGAGCCTGAGCCCCGGCGAGAGGGCCACCCTGA | |
| | GCTGCAGGGCCAGCCAGAGCGTGAGCAGCTACCTGGCCTGGTAC | |
| | CAGCAGAAGCCCGGCCAGGCCCCCAGGCTGCTGATCTACGACGC | |
| | CAGCAACAGGGCCACCGGCATCCCCGCCAGGTTCAGCGGCAGCG | |
| | GCAGCGGCACCGACTTCACCCTGACCATCAGCAGCCTGGAGCCCG | |
| | AGGACTTCGCCGTGTACTACTGCCAGCAGGTGGACAACTACCCCC | |
| | CCACCTTCGGCTGCGGCACCAAGGTGGAGATCAAG [SEQ ID | |
| | NO: 504] | |
| ADI-10165 | EVQLVESGGGLVQPGGSLRLSC | DIQMTQSPSSLSASVGDRVTITCR |
| [Ab19] | AASGFTFSSYWMSWVRQAPGK | ASQSISSYLNWYQQKPGKAPKLL |
| | GLEWVANIKQDGSEKYYVDSV | IYAASSLQSGVPSRFSGSGSGTDF |
| | KGRFTISRDNAKNSLYLQMNSL | TLTISSLQPEDFATYYCQQVYDT |
| | RAEDTAVYYCARDVGPGIAYQ | PLTFGGGTKVEIK [SEQ ID |
| | GHFDYWGQGTLVTVSS [SEQ ID | NO: 283] |
| | NO: 282] | CDR1: RASQSISSYLN [SEQ ID |
| | CDR1: SYWMS [SEQ ID NO: 352] | NO: 355] |
| | or FTFSSYWMS [SEQ ID NO: 544] | CDR2: AASSLQS [SEQ ID NO: 356] |
| | (non-Kabat) | CDR3: QQVYDTPLT [SEQ ID |
| | CDR2: NIKQDGSEKYYVDSVKG | NO: 357] |
| | [SEQ ID NO: 353] | |
| | CDR3: DVGPGIAYQGHFDY | |
| | [SEQ ID NO: 354] or | |
| | ARDVGPGIAYQGHFDY [SEQ ID | |
| | NO: 545](non-Kabat) | |
| scFv of | DIQMTQSPSSLSASVGDRVTITCRASQSISSYLNWYQQKPGKAPKLLI | |
| Ab19 | YAASSLQSGVPSRFSGSGSGTDFTLTISSLQPEDFATYYCQQVYDTPL | |
| | TFG [symbol] GTKVEIKGGGGSGGGGSGGGGSGGGGSEVQLVESGGGLVQ | |
| | PGGSLRLSCAASGFTFSSYWMSWVRQAPGK [symbol] LEWVANIKQDGSEKY | |
| | YVDSVKGRFTISRDNAKNSLYLQMNSLRAEDTAVYYCARDVGPGIA | |
| | YQGHFDYWGQGTLVTVSS [SEQ ID NO: 463] | |
| | EVQLVESGGGLVQPGGSLRLSCAASGFTFSSYWMSWVRQAPGK [symbol] LE | |
| | WVANIKQDGSEKYYVDSVKGRFTISRDNAKNSLYLQMNSLRAEDTA | |
| | VYYCARDVGPGIAYQGHFDYWGQGTLVTVSSGGGGSGGGGSGGG | |
| | GSGGGGSDIQMTQSPSSLSASVGDRVTITCRASQSISSYLNWYQQKP | |
| | GKAPKLLIYAASSLQSGVPSRFSGSGSGTDFTLTISSLQPEDFATYYCQ | |
| | QVYDTPLTFG [symbol] GTKVEIK [SEQ ID NO: 464] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCA | |
| sequence | GCAGCTACCTGAACTGGTACCAGCAGAAGCCCGGCAAGGCCCCC | |
| of Ab19 | AAGCTGCTGATCTACGCCGCCAGCAGCCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGTGTACGACACCCCCCTGACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCGGGCAAGTGCCTGGAGTGGGTGGCCAAC | |
| | ATCAAGCAGGACGGCAGCGAGAAGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGACGTGGGCCCCGGCATCGCCTACCAGGGCCAC | |
| | TTCGACTACTGGGGCCAGGGCACCCTGGTGACCGTGAGCAGC | |
| | [SEQ ID NO: 505] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAAGCAGGACGGCAGCGAGAAGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGACGTGGGCCCCGGC | |
| | ATCGCCTACCAGGGCCACTTCGACTACTGGGGCCAGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCCAGATGA | |
| | CCCAGAGCCCCAGCAGCCTGAGCGCCAGCGTGGGCGACAGGGTG | |
| | ACCATCACCTGCAGGGCCAGCCAGAGCATCAGCAGCTACCTGAA | |
| | CTGGTACCAGCAGAAGCCCGGCAAGGCCCCCAAGCTGCTGATCTA | |
| | CGCCGCCAGCAGCCTGCAGAGCGGCGTGCCCAGCAGGTTCAGCG | |
| | GCAGCGGCAGCGGCACCGACTTCACCCTGACCATCAGCAGCCTGC | |
| | AGCCCGAGGACTTCGCCACCTACTACTGCCAGCAGGTGTACGACA | |
| | CCCCCCTGACCTTCGGCTGCGGCACCAAGGTGGAGATCAAG [SEQ | |
| | ID NO: 506] | |
| ADI-10167 | QLQLQESGPGLVKPSETLSLTCT | EIVLTQSPATLSLSPGERATLSCR |
| [Ab20] | VSGGSISSSSYYWGWIRQPPGK | ASQSVSSYLAWYQQKPGQAPRL |
| | GLEWIGSIYYSGSTYYNPSLKSR | LIYDASNRATGIPARFSGSGSGTD |
| | VTISVDTSKNQFSLKLSSVTAAD | FTLTISSLEPEDFAVYYCQQYDN |
| | TAVYYCARETAHDVHGMDVW | LPTFGGGTKVEIK [SEQ ID |
| | GQGTTVTVSS [SEQ ID NO: 284] | NO: 285] |
| | CDR1: SSSYYWG [SEQ ID | CDR1: RASQSVSSYLA [SEQ ID |
| | NO: 358] or GSISSSSYYWG [SEQ | NO: 361] |
| | ID NO: 546](non-Kabat) | CDR2: DASNRAT [SEQ ID |
| | CDR2: SIYYSGSTYYNPSLKS | NO: 362] |
| | [SEQ ID NO: 359] | CDR3: QQYDNLPT [SEQ ID |
| | CDR3: ETAHDVHGMDV [SEQ ID | NO: 363] |
| | NO: 360] or ARETAHDVHGMDV | |
| | [SEQ ID NO: 547](non-Kabat) | |
| scFv of | EIVLTQSPATLSLSPGERATLSCRASQSVSSYLAWYQQKPGQAPRLLI | |
| Ab20 | YDASNRATGIPARFSGSGSGTDFTLTISSLEPEDFAVYYCQQYDNLPT | |
| | FG [symbol] GTKVEIKGGGGSGGGGSGGGGSGGGGSQLQLQESGPGLVKPS | |
| | ETLSLTCTVSGGSISSSSYYWGWIRQPPGK [symbol] LEWIGSIYYSGSTYYNP | |
| | SLKSRVTISVDTSKNQFSLKLSSVTAADTAVYYCARETAHDVHGMD | |
| | VWGQGTTVTVSS [SEQ ID NO: 465] | |
| | QLQLQESGPGLVKPSETLSLTCTVSGGSISSSSYYWGWIRQPPGK [symbol] LE | |
| | WIGSIYYSGSTYYNPSLKSRVTISVDTSKNQFSLKLSSVTAADTAVYY | |
| | CARETAHDVHGMDVWGQGTTVTVSSGGGGSGGGGSGGGGSGGG | |
| | GSEIVLTQSPATLSLSPGERATLSCRASQSVSSYLAWYQQKPGQAPRL | |
| | LIYDASNRATGIPARFSGSGSGTDFTLTISSLEPEDFAVYYCQQYDNLP | |
| | TFG [symbol] GTKVEIK [SEQ ID NO: 466] | |
| Exemplary | GAGATCGTGCTGACCCAGAGCCCCGCCACCCTGAGCCTGAGCCCC | |
| nucleotide | GGCGAGAGGGCCACCCTGAGCTGCAGGGCCAGCCAGAGCGTGAG | |
| sequence | CAGCTACCTGGCCTGGTACCAGCAGAAGCCCGGCCAGGCCCCCA | |
| of Ab20 | GGCTGCTGATCTACGACGOCAGCAACAGGGCCACCGGCATCCCCG | |
| scFv | CCAGGTTCAGCGGCAGCGGCAGCGGCACCGACTTCACCCTGACCA | |
| | TCAGCAGCCTGGAGCCCGAGGACTTCGCCGTGTACTACTGCCAGC | |
| | AGTACGACAACCTGCCCACCTTCGGCTGCGGCACCAAGGTGGAG | |
| | ATCAAGGGCGGCGGCGGCAGCGGCGGCGGCGGCAGCGGCGGCGG | |
| | CGGCAGCGGCGGCGGCGGCAGCCAGCTTGCAGCTGCAGGAGAGCG | |
| | GCCCCGGCCTGGTGAAGCCCAGCGAGACCCTGAGCCTGACCTGCA | |
| | CCGTGAGCGGCGGCAGCATCAGCAGCAGCAGCTACTACTGGGGC | |
| | TGGATCAGGCAGCCCCCCGGCAAGTGCCTGGAGTGGATCGGCAG | |
| | CATCTACTACAGCGGCAGCACCTACTACAACCCCAGCCTGAAGAG | |
| | CAGGGTGACCATCAGCGTGGACACCAGCAAGAACCAGTTCAGCC | |
| | TGAAGCTGAGCAGCGTGACCGCCGCCGACACCGCCGTGTACTACT | |
| | GCGCCAGGGAGACCGCCCACGACGTGCACGGCATGGACGTGTGG | |
| | GGCCAGGGCACCACCGTGACCGTGAGCAGC [SEQ ID NO: 507] | |
| | CAGCTGCAGCTGCAGGAGAGCGGCCCCGGCCTGGTGAAGCCCAG | |
| | CGAGACCCTGAGCCTGACCTGCACCGTGAGCGGCGGCAGCATCA | |
| | GCAGCAGCAGCTACTACTGGGGCTGGATCAGGCAGCCCCCCGGC | |
| | AAGTGCCTGGAGTGGATCGGCAGCATCTACTACAGCGGCAGCAC | |
| | CTACTACAACCCCAGCCTGAAGAGCAGGGTGACCATCAGCGTGG | |
| | ACACCAGCAAGAACCAGTTCAGCCTGAAGCTGAGCAGCGTGACC | |
| | GCCGCCGACACCGCCGTGTACTACTGCGCCAGGGAGACCGCCCAC | |
| | GACGTGCACGGCATGGACGTGTGGGGCCAGGGCACCACCGTGAC | |
| | CGTGAGCAGCGGCGGCGGCGGCAGCGGCGGCGGCGGCAGCGGCG | |
| | GCGGCGGCAGCGGCGGCGGCGGCAGCGAGATCGTGCTGACCCAG | |
| | AGCCCCGCCACCCTGAGCCTGAGCCCCGGCGAGAGGGCCACCCT | |
| | GAGCTGCAGGGCCAGCCAGAGCGTGAGCAGCTACCTGGCCTGGT | |
| | ACCAGCAGAAGCCCGGCCAGGCCCCCAGGCTGCTGATCTACGAC | |
| | GCCAGCAACAGGGCCACCCKSCATCCCCGCCAGGTTCAGCGGCAG | |
| | CGGCAGCGGCACCGACTTCACCCTGACCATCAGCAGCCTGGAGCC | |
| | CGAGGACTTCGCCGTGTACTACTGCCAGCAGTACGACAACCTGCC | |
| | CACCTTCGGCTGCGGCACCAAGGTGGAGATCAAG [SEQ ID | |
| | NO: 508] | |
| ADI-10168 | QVQLVQSGAEVKKPGSSVKVSC | EIVLTQSPATLSLSPGERATLSCR |
| [Ab21] | KASGGTFSSYAISWVRQAPGQG | ASQSVSSYLAWYQQKPGQAPRL |
| | LEWMGSIIPIFGTANYAQKFQGR | LIYDASKRATGIPARFSGSGSGTD |
| | VTITADESTSTAYMELSSLRSED | FTLTISSLEPEDFAVYYCQQSSNH |
| | TAVYYCAREVGYGWYTKIAFDI | PSTFGGGTKVEIK [SEQ ID |
| | WGQGTMVTVSS [SEQ ID | NO: 287] |
| | NO: 286] | CDR1: RASQSVSSYLA [SEQ ID |
| | CDR1: SYAIS [SEQ ID NO: 364] or | NO: 367] |
| | GTFSSYAIS [SEQ ID NO: 548] | CDR2: DASKRAT [SEQ ID |
| | (non-Kabat) | NO: 368] |
| | CDR2: SIIPIFGTANYAQKFQG | CDR3: QQSSNHPST [SEQ ID |
| | [SEQ ID NO: 365] | NO: 369] |
| | CDR3: EVGYGWYTKIAFDI [SEQ | |
| | ID NO: 366] or | |
| | AREVGYGWYTKIAFDI [SEQ ID | |
| | NO: 549](non-Kabat) | |
| scFv of | EIVLTQSPATLSLSPGERATLSCRASQSVSSYLAWYQQKPGQAPRLLI | |
| Ab21 | YDASKRATGIPARFSGSGSGTDFTLTISSLEPEDFAVYYCQQSSNHPST | |
| | FG [symbol] GTKVEIKGGGGSGGGGSGGGGSGGGGSQVQLVQSGAEVKKP | |
| | GSSVKVSCKASGGTFSSYAISWVRQAPGQ [symbol] LEWMGSIIPIFGTANYA | |
| | QKFQGRVTITADESTSTAYMELSSLRSEDTAVYYCAREVGYGWYTK | |
| | IAFDIWGQGTMVTVSS [SEQ ID NO: 467] | |
| | QVQLVQSGAEVKKPGSSVKVSCKASGGTFSSYAISWVRQAPGQ [symbol] LE | |
| | WMGSIIPIFGTANYAQKFQGRVTITADESTSTAYMELSSLRSEDTAVY | |
| | YCAREVGYGWYTKIAFDIWGQGTMVTVSSGGGGSGGGGSGGGGS | |
| | GGGGSEIVLTQSPATLSLSPGERATLSCRASQSVSSYLAWYQQKPGQ | |
| | APRLLIYDASKRATGIPARFSGSGSGTDFTLTISSLEPEDFAVYYCQQS | |
| | SNHPSTFG [symbol] GTKVEIK [SEQ ID NO: 468] | |
| Exemplary | GAGATCGTGCTGACCCAGAGCCCCGCCACCCTGAGCCTGAGCCCC | |
| nucleotide | GGCGAGAGGGCCACCCTGAGCTGCAGGGCCAGCCAGAGCGTGAG | |
| sequence | CAGCTACCTGGCCTGGTACCAGCAGAAGCCCGGCCAGGCCCCCA | |
| of Ab21 | GGCTGCTGATCTACGACGCCAGCAAGAGGGCCACCGGCATCCCC | |
| scFv | GCCAGGTTCAGCGGCAGCGGCAGCGGCACCGACTTCACCCTGACC | |
| | ATCAGCAGCCTGGAGCCCGAGGACTTCGCCGTGTACTACTGCCAG | |
| | CAGAGCAGCAACCACCCCAGCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCCAGGTGCAGCTGGTGCA | |
| | GAGCGGCGCCGAGGTGAAGAAGCCCGGCAGCAGCGTGAAGGTGA | |
| | GCTGCAAGGCCAGCGGCGGCACCTTCAGCAGCTACGCCATCAGCT | |
| | GGGTGAGGCAGGCCCCCGGCCAGTGCCTGGAGTGGATGGGCAGC | |
| | ATCATCCCCATCTTCGGCACCGCCAACTACGCCCAGAAGTTCCAG | |
| | GGCAGGGTGACCATCACCGCCGACGAGAGCACCAGCACCGCCTA | |
| | CATGGAGCTGAGCAGCCTGAGGAGCGAGGACACCGCCGTGTACT | |
| | ACTGCGCCAGGGAGGTGGGCTACGGCTGGTACACCAAGATCGCC | |
| | TTCGACATCTGGGGCCAGGGCACCATGGTGACCGTGAGCAGC | |
| | [SEQ ID NO: 509] | |
| | CAGGTGCAGCTGGTGCAGAGCGGCGCCGAGGTGAAGAAGCCCGG | |
| | CAGCAGCGTGAAGGTGAGCTGCAAGGCCAGCGGCGGCACCTTCA | |
| | GCAGCTACGCCATCAGCTGGGTGAGGCAGGCCCCCGGCCAGTGC | |
| | CTGGAGTGGATGGGCAGCATCATCCCCATCTTCGGCACCGCCAAC | |
| | TACGCCCAGAAGTTCCAGGGCAGGGTGACCATCACCGCCGACGA | |
| | GAGCACCAGCACCGCCTACATGGAGCTGAGCAGCCTGAGGAGCG | |
| | AGGACACCGCCGTGTACTACTGCGCCAGGGAGGTGGGCTACGGC | |
| | TGGTACACCAAGATCGCCTTCGACATCTGGGGCCAGGGCACCATG | |
| | GTGACCGTGAGCAGCGGCGGCGGCGGCAGCGGCGGCGGCGGCAG | |
| | CGGCGGCGGCGGCAGCGGCGGCGGCGGCAGCGAGATCGTGCTGA | |
| | CCCAGAGCCCCGCCACCCTGAGCCTGAGCCCCGGCGAGAGGGCC | |
| | ACCCTGAGCTGCAGGGCCAGCCAGAGCGTGAGCAGCTACCTGGC | |
| | CTGGTACCAGCAGAAGCCCGGCCAGGCCCCCAGGCTGCTGATCTA | |
| | CGACGCCAGCAAGAGGGCCACCGGCATCCCCGCCAGGTTCAGCG | |
| | GCAGCGGCAGCGGCACCGACTTCACCCTGACCATCAGCAGCCTGG | |
| | AGCCCGAGGACTTCGCCGTGTACTACTGCCAGCAGAGCAGCAACC | |
| | ACCCCAGCACCTTCGGCTGCGGCACCAAGGTGGAGATCAAG [SEQ | |
| | ID NO: 510] | |
| ADI-10173 | QVQLVQSGAEVKKPGASVKVS | DIVMTQSPLSLPVTPGEPASISCR |
| [Ab22] | CKASGYTFTSYYMHWVRQAPG | SSQSLLHSNGYNYLDWYLQKPG |
| | QGLEWMGIINPSGGSTTYAQKF | QSPQLLIYLGSNRASGVPDRFSGS |
| | QGRVTMTRDTSTSTVYMELSSL | GSGTDFTLKISRVEAEDVGVYYC |
| | RSEDTAVYYCAREAADGFVGE | MQALGVPLTFGGGTKVEIK [SEQ |
| | RYFDLWGRGTLVTVSS [SEQ ID | ID NO: 289] |
| | NO: 288] | CDR1: RSSQSLLHSNGYNYLD |
| | CDR1: SYYMH [SEQ ID NO: 370] | [SEQ ID NO: 373] |
| | or YTFTSYYMH [SEQ ID NO: 550] | CDR2: LGSNRAS [SEQ ID NO: 374] |
| | (non-Kabat) | CDR3: MQALGVPLT [SEQ ID |
| | CDR2: IINPSGGSTTYAQKFQG | NO: 375] |
| | [SEQ ID NO: 371] | |
| | CDR3: EAADGFVGERYFDL | |
| | [SEQ ID NO: 372] or | |
| | AREAADGFVGERYFDL [SEQ ID | |
| | NO: 551](non-Kabat) | |
| scFv of | DIVMTQSPLSLPVTPGEPASISCRSSQSLLHSNGYNYLDWYLQKPGQS | |
| Ab22 | PQLLIYLGSNRASGVPDRFSGSGSGTDFTLKISRVEAEDVGVYYCMQ | |
| | ALGVPLTFG [symbol] GTKVEIKGGGGSGGGGSGGGGSGGGGSQVQLVQS | |
| | GAEVKKPGASVKVSCKASGYTFTSYYMHWVRQAPGQ [symbol] LEWMGIIN | |
| | PSGGSTTYAQKFQGRVTMTRDTSTSTVYMELSSLRSEDTAVYYCAR | |
| | EAADGFVGERYFDLWGRGTLVTVSS [SEQ ID NO: 469] | |
| | QVQLVQSGAEVKKPGASVKVSCKASGYTFTSYYMHWVRQAPGQ [symbol] L | |
| | EWMGIINPSGGSTTYAQKFQGRVTMTRDTSTSTVYMELSSLRSEDTA | |
| | VYYCAREAADGFVGERYFDLWGRGTLVTVSSGGGGSGGGGSGGG | |
| | GSGGGGSDIVMTQSPLSLPVTPGEPASISCRSSQSLLHSNGYNYLDW | |
| | YLQKPGQSPQLLIYLGSNRASGVPDRFSGSGSGTDFTLKISRVEAEDV | |
| | GVYYCMQALGVPLTFG [symbol] GTKVEIK [SEQ ID NO: 470] | |
| Exemplary | GACATCGTGATGACCCAGAGCCCCCTGAGCCTGCCCGTGACCCCC | |
| nucleotide | GGCGAGCCCGCCAGCATCAGCTGCAGGAGCAGCCAGAGCCTGCT | |
| sequence | GCACAGCAACGGCTACAACTACCTGGACTGGTACCTGCAGAAGC | |
| of Ab22 | CCGGCCAGAGCCCCCAGCTGCTGATCTACCTGGGCAGCAACAGG | |
| scFv | GCCAGCGGCGTGCCCGACAGGTTCAGCGGCAGCGGCAGCGGCAC | |
| | CGACTTCACCCTGAAGATCAGCAGGGTGGAGGCCGAGGACGTGG | |
| | GCGTGTACTACTGCATGCAGGCCCTGGGCGTGCCCCTGACCTTCG | |
| | GCTGCGGCACCAAGGTGGAGATCAAGGGCGGCGGCGGCAGCGGC | |
| | GGCGGCGGCAGCGGCGGCGGCGGCAGCGGCGGCGGCGGCAGCCA | |
| | GGTGCAGCTGGTGCAGAGCGGCGCCGAGGTGAAGAAGCCCGGCG | |
| | CCAGCGTGAAGGTGAGCTGCAAGGCCAGCGGCTACACCTTCACC | |
| | AGCTACTACATGCACTGGGTGAGGCAGGCCCCCGGCCAGTGCCTG | |
| | GAGTGGATGGGCATCATCAACCCCAGCGGCGGCAGCACCACCTA | |
| | CGCCCAGAAGTTCCAGGGCAGGGTGACCATGACCAGGGACACCA | |
| | GCACCAGCACCGTGTACATGGAGCTGAGCAGCCTGAGGAGCGAG | |
| | GACACCGCCGTGTACTACTGCGCCAGGGAGGCCGCCGACGGCTTC | |
| | GTGGGCGAGAGGTACTTCGACCTGTGGGGCAGGGGCACCCTGGT | |
| | GACCGTGAGCAGC [SEQ ID NO: 511] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCA | |
| | CCAGCTACTACATGCACTGGGTGAGGCAGGCCCCCGGCCAGTGCC | |
| | TGGAGTGGATGGGCATCATCAACCCCAGCGGCGGCAGCACCACC | |
| | TACGCCCAGAAGTTCCAGGGCAGGGTGACCATGACCAGGGACAC | |
| | CAGCACCAGCACCGTGTACATGGAGCTGAGCAGCCTGAGGAGCG | |
| | AGGACACCGCCGTGTACTACTGCGCCAGGGAGGCCGCCGACGGC | |
| | TTCGTGGGCGAGAGGTACTTCGACCTGTGGGGCAGGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCGTGATGA | |
| | CCCAGAGCCCCCTGAGCCTGCCCGTGACCCCCGGCGAGCCCGCCA | |
| | GCATCAGCTGCAGGAGCAGCCAGAGCCTGCTGCACAGCAACGGC | |
| | TACAACTACCTGGACTGGTACCTGCAGAAGCCCGGCCAGAGCCCC | |
| | CAGCTGCTGATCTACCTGGGCAGCAACAGGGCCAGCGGCGTGCCC | |
| | GACAGGTTCAGCGGCAGCGGCAGCGGCACCGACTTCACCCTGAA | |
| | GATCAGCAGGGTGGAGGCCGAGGACGTGGGCGTGTACTACTGCA | |
| | TGCAGGCCCTGGGCGTGCCCCTGACCTTCGGCTGCGGCACCAAGG | |
| | TGGAGATCAAG [SEQ ID NO: 512] | |
| ADI-11802 | QVQLVQSGAEVKKPGASVKVS | DIVMTQSPLSLPVTPGEPASISCR |
| [Ab23] | CKASGYTFSGYYMHWVRQAPG | SSQSLLYSNGYNYLDWYLQKPG |
| | QGLEWMGMINPYGGSTRYAQK | QSPQLLIYLGSNRASGVPDRFSGS |
| | FQGRVTMTRDTSTSTVYMELSS | GSGTDFTLKISRVEAEDVGVYYC |
| | LRSEDTAVYYCAREAADGFVGE | MQDVALPITFGGGTKVEIK [SEQ |
| | RYFDLWGRGTLVTVSS [SEQ ID | ID NO: 291] |
| | NO: 290] | CDR1: RSSQSLLYSNGYNYLD |
| | CDR1: GYYMH [SEQ ID NO: 376] | [SEQ ID NO: 379] |
| | or YTFSGYYMH [SEQ ID | CDR2: LGSNRAS [SEQ ID NO: 380] |
| | NO: 552](non-Kabat) | CDR3: MQDVALPIT [SEQ ID |
| | CDR2: MINPYGGSTRYAQKFQG | NO: 381] |
| | [SEQ ID NO: 377] | |
| | CDR3: EAADGFVGERYFDL | |
| | [SEQ ID NO: 378] or | |
| | AREAADGFVGERYFDL [SEQ ID | |
| | NO: 553](non-Kabat) | |
| scFv of | DIVMTQSPLSLPVTPGEPASISCRSSQSLLYSNGYNYLDWYLQKPGQS | |
| Ab23 | PQLLIYLGSNRASGVPDRFSGSGSGTDFTLKISRVEAEDVGVYYCMQ | |
| | DVALPITFG [symbol] GTKVEIKGGGGSGGGGSGGGGSGGGGSQVQLVQSG | |
| | AEVKKPGASVKVSCKASGYTFSGYYMHWVRQAPGQ [symbol] LEWMGMIN | |
| | PYGGSTRYAQKFQGRVTMTRDTSTSTVYMELSSLRSEDTAVYYCAR | |
| | EAADGFVGERYFDLWGRGTLVTVSS [SEQ ID NO: 471] | |
| | QVQLVQSGAEVKKPGASVKVSCKASGYTFSGYYMHWVRQAPGQ [symbol] L | |
| | EWMGMINPYGGSTRYAQKFQGRVTMTRDTSTSTVYMELSSLRSEDT | |
| | AVYYCAREAADGFVGERYFDLWGRGTLVTVSSGGGGSGGGGSGG | |
| | GGSGGGGSDIVMTQSPLSLPVTPGEPASISCRSSQSLLYSNGYNYLD | |
| | WYLQKPGQSPQLLIYLGSNRASGVPDRFSGSGSGTDFTLKISRVEAED | |
| | VGVYYCMQDVALPITFG [symbol] GTKVEIK [SEQ ID NO: 472] | |
| Exemplary | GACATCGTGATGACCCAGAGCCCCCTGAGCCTGCCCGTGACCCCC | |
| nucleotide | GGCGAGCCCGCCAGCATCAGCTGCAGGAGCAGCCAGAGCCTGCT | |
| sequence | GTACAGCAACGGCTACAACTACCTGGACTGGTACCTGCAGAAGCC | |
| of Ab23 | CGGCCAGAGCCCCCAGCTGCTGATCTACCTGGGCAGCAACAGGG | |
| scFv | CCAGCGGCGTGCCCGACAGGTTCAGCGGCAGCGGCAGCGGCACC | |
| | GACTTCACCCTGAAGATCAGCAGGGTGGAGGCCGAGGACGTGGG | |
| | CGTGTACTACTGCATGCAGGACGTGGCCCTGCCCATCACCTTCGG | |
| | CTGCGGCACCAAGGTGGAGATCAAGGGCGGCGGCGGCAGCGGCG | |
| | GCGGCGGCAGCGGCGGCGGCGGCAGCGGCGGCGGCGGCAGCCAG | |
| | GTGCAGCTGGTGCAGAGCGGCGCCGAGGTGAAGAAGCCCGGCGC | |
| | CAGCGTGAAGGTGAGCTGCAAGGCCAGCGGCTACACCTTCAGCG | |
| | GCTACTACATGCACTGGGTGAGGCAGGCCCCCGGCCAGTGCCTGG | |
| | AGTGGATGGGCATGATCAACCCCTACGGCGGCAGCACCAGGTAC | |
| | GCCCAGAAGTTCCAGGGCAGGGTGACCATGACCAGGGACACCAG | |
| | CACCAGCACCGTGTACATGGAGCTGAGCAGCCTGAGGAGCGAGG | |
| | ACACCGCCGTGTACTACTGCGCCAGGGAGGCCGCCGACGGCTTCG | |
| | TGGGCGAGAGGTACTTCGACCTGTGGGGCAGGGGCACCCTGGTG | |
| | ACCGTGAGCAGC [SEQ ID NO: 513] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCA | |
| | GCGGCTACTACATGCACTGGGTGAGGCAGGCCCCCGGCCAGTGCC | |
| | TGGAGTGGATGGGCATGATCAACCCCTACGGCGGCAGCACCAGG | |
| | TACGCCCAGAAGTTCCAGGGCAGGGTGACCATGACCAGGGACAC | |
| | CAGCACCAGCACCGTGTACATGGAGCTGAGCAGCCTGAGGAGCG | |
| | AGGACACCGCCGTGTACTACTGCGCCAGGGAGGCCGCCGACGGC | |
| | TTCGTGGGCGAGAGGTACTTCGACCTGTGGGGCAGGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCGTGATGA | |
| | CCCAGAGCCCCCTGAGCCTGCCCGTGACCCCCGGCGAGCCCGCCA | |
| | GCATCAGCTGCAGGAGCAGCCAGAGCCTGCTGTACAGCAACGGC | |
| | TACAACTACCTGGACTGGTACCTGCAGAAGCCCGGCCAGAGCCCC | |
| | CAGCTGCTGATCTACCTGGGCAGCAACAGGGCCAGCGGCGTGCCC | |
| | GACAGGTTCAGCGGCAGCGGCAGCGGCACCGACTTCACCCTGAA | |
| | GATCAGCAGGGTGGAGGCCGAGGACGTGGGCGTGTACTACTGCA | |
| | TGCAGGACGTGGCCCTGCCCATCACCTTCGGCTGCGGCACCAAGG | |
| | TGGAGATCAAG [SEQ ID NO: 514] | |
| ADI-11812 | QVQLVQSGAEVKKPGASVKVS | DIQMTQSPSSVSASVGDRVTITC |
| [Ab24] | CKASGYTFEIYYMHWVRQAPG | RASQGIDSWLAWYQQKPGKAPK |
| | QGLEWMGIINPSSGSTVYAQKF | LLIYAASSLQSGVPSRFSGSGSGT |
| | QGRVTMTRDTSTSTVYMELSSL | DFTLTISSLQPEDFATYYCQQAHS |
| | RSEDTAVYYCARGAGYDDEDM | YPLTFGGGTKVEIK [SEQ ID |
| | DVWGKGTTVTVSS [SEQ ID | NO: 293] |
| | NO: 292] | CDR1: RASQGIDSWLA [SEQ ID |
| | CDR1: IYYMH [SEQ ID NO: 382] | NO: 385] |
| | or YTFEIYYMH [SEQ ID NO: 554] | CDR2: AASSLQS [SEQ ID NO: 386] |
| | (non-Kabat) | CDR3: QQAHSYPLT [SEQ ID |
| | CDR2: IINPSSGSTVYAQKFQG | NO: 387] |
| | [SEQ ID NO: 383] | |
| | CDR3: GAGYDDEDMDV [SEQ | |
| | ID NO: 384] or | |
| | ARGAGYDDEDMDV [SEQ ID | |
| | NO: 555](non-Kabat) | |
| scFv of | DIQMTQSPSSVSASVGDRVTITCRASQGIDSWLAWYQQKPGKAPKLL | |
| Ab24 | IYAASSLQSGVPSRFSGSGSGTDFTLTISSLQPEDFATYYCQQAHSYPL | |
| | TFG [symbol] GTKVEIKGGGGSGGGGSGGGGSGGGGSQVQLVQSGAEVKK | |
| | PGASVKVSCKASGYTFEIYYMHWVRQAPGQ [symbol] LEWMGIINPSSGSTV | |
| | YAQKFQGRVTMTRDTSTSTVYMELSSLRSEDTAVYYCARGAGYDD | |
| | EDMDVWGKGTTVTVSS [SEQ ID NO: 473] | |
| | QVQLVQSGAEVKKPGASVKVSCKASGYTFEIYYMHWVRQAPGQ [symbol] L | |
| | EWMGIINPSSGSTVYAQKFQGRVTMTRDTSTSTVYMELSSLRSEDTA | |
| | VYYCARGAGYDDEDMDVWGKGTTVTVSSGGGGSGGGGSGGGGS | |
| | GGGGSDIQMTQSPSSVSASVGDRVTITCRASQGIDSWLAWYQQKPG | |
| | KAPKLLIYAASSLQSGVPSRFSGSGSGTDFTLTISSLQPEDFATYYCQQ | |
| | AHSYPLTFG [symbol] GTKVEIK [SEQ ID NO: 474] | |
| Exemplary | GACATCCAGATGACCCAGAGCCCCAGCAGCGTGAGCGCCAGCGT | |
| nucleotide | GGGCGACAGGGTGACCATCACCTGCAGGGCCAGCCAGGGCATCG | |
| sequence | ACAGCTGGCTGGCCTGGTACCAGCAGAAGCCCGGCAAGGCCCCC | |
| of Ab24 | AAGCTGCTGATCTACGCCGCCAGCAGCCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGCCCACAGCTACCCCCTGACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCCAGGTGCAGCTGGTGCA | |
| | GAGCGGCGCCGAGGTGAAGAAGCCCGGCGCCAGCGTGAAGGTGA | |
| | GCTGCAAGGCCAGCGGCTACACCTTCGAGATCTACTACATGCACT | |
| | GGGTGAGGCAGGCCCCCGGCCAGTGCCTGGAGTGGATGGGCATC | |
| | ATCAACCCCAGCAGCGGCAGCACCGTGTACGCCCAGAAGTTCCA | |
| | GGGCAGGGTGACCATGACCAGGGACACCAGCACCAGCACCGTGT | |
| | ACATGGAGCTGAGCAGCCTGAGGAGCGAGGACACCGCCGTGTAC | |
| | TACTGCGCCAGGGGCGCCGGCTACGACGACGAGGACATGGACGT | |
| | GTGGGGCAAGGGCACCACCGTGACCGTGAGCAGC [SEQ ID | |
| | NO: 515] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCG | |
| | AGATCTACTACATGCACTGGGTGAGGCAGGCCCCCGGCCAGTGCC | |
| | TGGAGTGGATGGGCATCATCAACCCCAGCAGCGGCAGCACCGTG | |
| | TACGCCCAGAAGTTCCAGGGCAGGGTGACCATGACCAGGGACAC | |
| | CAGCACCAGCACCGTGTACATGGAGCTGAGCAGCCTGAGGAGCG | |
| | AGGACACCGCCGTGTACTACTGCGCCAGGGGCGCCGGCTACGAC | |
| | GACGAGGACATGGACGTGTGGGGCAAGGGCACCACCGTGACCGT | |
| | GAGCAGCGGCGGCGGCGGCAGCGGCGGCGGCGGCAGCGGCGGC | |
| | GGCGGCAGCGGCGGCGGCGGCAGCGACATCCAGATGACCCAGAG | |
| | CCCCAGCAGCGTGAGCGCCAGCGTGGGCGACAGGGTGACCATCA | |
| | CCTGCAGGGCCAGCCAGGGCATCGACAGCTGGCTGGCCTGGTACC | |
| | AGCAGAAGCCCGGCAAGGCCCCCAAGCTGCTGATCTACGCCGCC | |
| | AGCAGCCTGCAGAGCGGCGTGCCCAGCAGGTTCAGCGGCAGCGG | |
| | CAGCGGCACCGACTTCACCCTGACCATCAGCAGCCTGCAGCCCGA | |
| | GGACTTCGCCACCTACTACTGCCAGCAGGCCCACAGCTACCCCCT | |
| | GACCTTCGGCTGCGGCACCAAGGTGGAGATCAAG [SEQ ID | |
| | NO: 516] | |
| ADI-11825 | EVQLVESGGGLVQPGGSLRLSC | DIQMTQSPSSLSASVGDRVTITCR |
| [Ab25] | AASGFTFGGYWMSWVRQAPGK | ASQSIYNYLNWYQQKPGKAPKL |
| | GLEWVANINQDGSEEYYVDSV | LIYAASNLHSGVPSRFSGSGSGT |
| | KGRFTISRDNAKNSLYLQMNSL | DFTLTISSLQPEDFATYYCQQAFH |
| | RAEDTAVYYCAREANYYGNVG | VPITFGGGTKVEIK [SEQ ID |
| | DDYWGQGTLVTVSS [SEQ ID | NO: 295] |
| | NO: 294] | CDR1: RASQSIYNYLN [SEQ ID |
| | CDR1: GYWMS [SEQ ID NO: 388] | NO: 391] |
| | or FTFGGYWMS [SEQ ID | CDR2: AASNLHS [SEQ ID |
| | NO: 556](non-Kabat) | NO: 392] |
| | CDR2: NINQDGSEEYYVDSVKG | CDR3: QQAFHVPIT [SEQ ID |
| | [SEQ ID NO: 389] | NO: 393] |
| | CDR3: EANYYGNVGDDY [SEQ | |
| | ID NO: 390] or | |
| | AREANYYGNVGDDY [SEQ ID | |
| | NO: 557](non-Kabat) | |
| scFv of | DIQMTQSPSSLSASVGDRVTITCRASQSIYNYLNWYQQKPGKAPKLLI | |
| Ab25 | YAASNLHSGVPSRFSGSGSGTDFTLTISSLQPEDFATYYCQQAFHVPI | |
| | TFG [symbol] GTKVEIKGGGGSGGGGSGGGGSGGGGSEVQLVESGGGLVQ | |
| | PGGSLRLSCAASGFTFGGYWMSWVRQAPGK [symbol] LEWVANINQDGSEE | |
| | YYVDSVKGRFTISRDNAKNSLYLQMNSLRAEDTAVYYCAREANYY | |
| | GNVGDDYWGQGTLVTVSS [SEQ ID NO: 475] | |
| | EVQLVESGGGLVQPGGSLRLSCAASGFTFGGYWMSWVRQAPGK [symbol] L | |
| | EWVANINQDGSEEYYVDSVKGRFTISRDNAKNSLYLQMNSLRAEDT | |
| | AVYYCAREANYYGNVGDDYWGQGTLVTVSSGGGGSGGGGSGGG | |
| | GSGGGGSDIQMTQSPSSLSASVGDRVTITCRASQSIYNYLNWYQQKP | |
| | GKAPKLLIYAASNLHSGVPSRFSGSGSGTDFTLTISSLQPEDFATYYC | |
| | QQAFHVPITFG [symbol] GTKVEIK [SEQ ID NO: 476] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCT | |
| sequence | ACAACTACCTGAACTGGTACCAGCAGAAGCCCGGCAAGGCCCCC | |
| of Ab25 | AAGCTGCTGATCTACGCCGCCAGCAACCTGCACAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGCCTTCCACGTGCCCATCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCGGCGGCTACTGGATGAGCT | |
| | GGGTGAGGCAGGCCCCCGGCAAGTGCCTGGAGTGGGTGGCCAAC | |
| | ATCAACCAGGACGGCAGCGAGGAGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGAGGCCAACTACTACGGCAACGTGGGCGACGA | |
| | CTACTGGGGCCAGGGCACCCTGGTGACCGTGAGCAGC [SEQ ID | |
| | NO: 517] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCGG | |
| | CGGCTACTGGATGAGCTGGGTGAGGCAGGCCCCCGGCAAGTGCC | |
| | TGGAGTGGGTGGCCAACATCAACCAGGACGGCAGCGAGGAGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGCCAACTACTAC | |
| | GGCAACGTGGGCGACGACTACTGGGGCCAGGGCACCCTGGTGAC | |
| | CGTGAGCAGCGGCGGCGGCGGCAGCGGCGGCGGCGGCAGCGGCG | |
| | GCGGCGGCAGCGGCGGCGGCGGCAGCGACATCCAGATGACCCAG | |
| | AGCCCCAGCAGCCTGAGCGCCAGCGTGGGCGACAGGGTGACCAT | |
| | CACCTGCAGGGCCAGCCAGAGCATCTACAACTACCTGAACTGGTA | |
| | CCAGCAGAAGCCCGGCAAGGCCCCCAAGCTGCTGATCTACGCCG | |
| | CCAGCAACCTGCACAGCGGCGTGCCCAGCAGGTTCAGCGGCAGC | |
| | GGCAGCGGCACCGACTTCACCCTGACCATCAGCAGCCTGCAGCCC | |
| | GAGGACTTCGCCACCTACTACTGCCAGCAGGCCTTCCACGTGCCC | |
| | ATCACCTTCGGCTGCGGCACCAAGGTGGAGATCAAG [SEQ ID | |
| | NO: 518] | |
| ADI-11826 | EVQLVESGGGLVQPGGSLRLSC | DIQMTQSPSSLSASVGDRVTITCR |
| [Ab26] | AASGFTFPGYWMSWVRQAPGK | ASQSIYNYLNWYQQKPGKAPKL |
| | GLEWVANINQDGSEVYYVDSV | LIYAASSTQSGVPSRFSGSGSGTD |
| | KGRFTISRDNAKNSLYLQMNSL | FTLTISSLQPEDFATYYCQQAFHV |
| | RAEDTAVYYCAREANYYGNVG | PITFGGGTKVEIK [SEQ ID |
| | DDYWGQGTLVTVSS [SEQ ID | NO: 297] |
| | NO: 296] | CDR1: RASQSIYNYLN [SEQ ID |
| | CDR1: GYWMS [SEQ ID NO: 394] | NO: 397] |
| | or FTFPGYWMS [SEQ ID NO: 558] | CDR2: AASSTQS [SEQ ID NO: 398] |
| | (non-Kabat) | CDR3: QQAFHVPIT [SEQ ID |
| | CDR2: NINQDGSEVYYVDSVKG | NO: 399] |
| | [SEQ ID NO: 395] | |
| | CDR3: EANYYGNVGDDY [SEQ | |
| | ID NO: 396] or | |
| | AREANYYGNVGDDY [SEQ ID | |
| | NO: 559](non-Kabat) | |
| scFv of | DIQMTQSPSSLSASVGDRVTITCRASQSIYNYLNWYQQKPGKAPKLLI | |
| Ab26 | YAASSTQSGVPSRFSGSGSGTDFTLTISSLQPEDFATYYCQQAFHVPIT | |
| | FG [symbol] GTKVEIKGGGGSGGGGSGGGGSGGGGSEVQLVESGGGLVQP | |
| | GGSLRLSCAASGFTFPGYWMSWVRQAPGK [symbol] LEWVANINQDGSEVY | |
| | YVDSVKGRFTISRDNAKNSLYLQMNSLRAEDTAVYYCAREANYYG | |
| | NVGDDYWGQGTLVTVSS [SEQ ID NO: 477] | |
| | EVQLVESGGGLVQPGGSLRLSCAASGFTFPGYWMSWVRQAPGK [symbol] LE | |
| | WVANINQDGSEVYYVDSVKGRFTISRDNAKNSLYLQMNSLRAEDTA | |
| | VYYCAREANYYGNVGDDYWGQGTLVTVSSGGGGSGGGGSGGGG | |
| | SGGGGSDIQMTQSPSSLSASVGDRVTITCRASQSIYNYLNWYQQKPG | |
| | KAPKLLIYAASSTQSGVPSRFSGSGSGTDFTLTISSLQPEDFATYYCQQ | |
| | AFHVPITFG [symbol] GTKVEIK [SEQ ID NO: 478] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCT | |
| sequence | ACAACTACCTGAACTGGTACCAGCAGAAGCCCGGCAAGGCCCCC | |
| of Ab26 | AAGCTGCTGATCTACGCCGCCAGCAGCACCCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGCCTTCCACGTGCCCATCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCCCCGGCTACTGGATGAGCT | |
| | GGGTGAGGCAGGCCCCCGGCAAGTGCCTGGAGTGGGTGGCCAAC | |
| | ATCAACCAGGACGGCAGCGAGGTGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGAGGCCAACTACTACGGCAACGTGGGCGACGA | |
| | CTACTGGGGCCAGGGCACCCTGGTGACCGTGAGCAGC [SEQ ID | |
| | NO: 519] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCCC | |
| | CGGCTACTGGATGAGCTGGGTGAGGCAGGCCCCCGGCAAGTGCC | |
| | TGGAGTGGGTGGCCAACATCAACCAGGACGGCAGCGAGGTGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGAGGCCAACTACTAC | |
| | GGCAACGTGGGCGACGACTACTGGGGCCAGGGCACCCTGGTGAC | |
| | CGTGAGCAGCGGCGGCGGCGGCAGCGGCGGCGGCGGCAGCGGCG | |
| | GCGGCGGCAGCGGCGGCGGCGGCAGCGACATCCAGATGACCCAG | |
| | AGCCCCAGCAGCCTGAGCGCCAGCGTGGGCGACAGGGTGACCAT | |
| | CACCTGCAGGGCCAGCCAGAGCATCTACAACTACCTGAACTGGTA | |
| | CCAGCAGAAGCCCGGCAAGGCCCCCAAGCTGCTGATCTACGCCG | |
| | CCAGCAGCACCCAGAGCGGCGTGCCCAGCAGGTTCAGCGGCAGC | |
| | GGCAGCGGCACCGACTTCACCCTGACCATCAGCAGCCTGCAGCCC | |
| | GAGGACTTCGCCACCTACTACTGCCAGCAGGCCTTCCACGTGCCC | |
| | ATCACCTTCGGCTGCGGCACCAAGGTGGAGATCAAG [SEQ ID | |
| | NO: 520] | |
| ADI-11828 | EVQLVESGGGLVQPGGSLRLSC | DIQMTQSPSSLSASVGDRVTITCR |
| [Ab27] | AASGFTFSSYWMSWVRQAPGK | ASQSIYYYLNWYQQKPGKAPKL |
| | GLEWVANINQDGSEVYYVDSV | LIYAASSRQSGVPSRFSGSGSGTD |
| | KGRFTISRDNAKNSLYLQMNSL | FTLTISSLQPEDFATYYCQQVYD |
| | RAEDTAVYYCARDVGPGIAYQ | TPLTFGGGTKVEIK [SEQ ID |
| | GHFDYWGQGTLVTVSS [SEQ ID | NO: 299] |
| | NO: 298] | CDR1: RASQSIYYYLN [SEQ ID |
| | CDR1: SYWMS [SEQ ID NO: 400] | NO: 403] |
| | or FTFSSYWMS [SEQ ID NO: 560] | CDR2: AASSRQS [SEQ ID NO: 404] |
| | (non-Kabat) | CDR3: QQVYDTPLT [SEQ ID |
| | CDR2: NINQDGSEVYYVDSVKG | NO: 405] |
| | [SEQ ID NO: 401] | |
| | CDR3: DVGPGIAYQGHFDY | |
| | [SEQ ID NO: 402] or | |
| | ARDVGPGIAYQGHFDY [SEQ ID | |
| | NO: 561](non-Kabat) | |
| scFv of | DIQMTQSPSSLSASVGDRVTITCRASQSIYYYLNWYQQKPGKAPKLLI | |
| Ab27 | YAASSRQSGVPSRFSGSGSGTDFTLTISSLQPEDFATYYCQQVYDTPL | |
| | TFG [symbol] GTKVEIKGGGGSGGGGSGGGGSGGGGSEVQLVESGGGLVQ | |
| | PGGSLRLSCAASGFTFSSYWMSWVRQAPGK [symbol] LEWVANINQDGSEVY | |
| | YVDSVKGRFTISRDNAKNSLYLQMNSLRAEDTAVYYCARDVGPGIA | |
| | YQGHFDYWGQGTLVTVSS [SEQ ID NO: 479] | |
| | EVQLVESGGGLVQPGGSLRLSCAASGFTFSSYWMSWVRQAPGK [symbol] LE | |
| | WVANINQDGSEVYYVDSVKGRFTISRDNAKNSLYLQMNSLRAEDTA | |
| | VYYCARDVGPGIAYQGHFDYWGQGTLVTVSSGGGGSGGGGSGGG | |
| | GSGGGGSDIQMTQSPSSLSASVGDRVTITCRASQSIYYYLNWYQQKP | |
| | GKAPKLLIYAASSRQSGVPSRFSGSGSGTDFTLTISSLQPEDFATYYCQ | |
| | QVYDTPLTFG [symbol] GTKVEIK [SEQ ID NO: 480] | |
| Exemplary | GACATCCAGATGACCCAGAGCCCCAGCAGCCTGAGCGCCAGCGT | |
| nucleotide | GGGCGACAGGGTGACCATCACCTGCAGGGCCAGCCAGAGCATCT | |
| sequence | ACTACTACCTGAACTGGTACCAGCAGAAGCCCGGCAAGGCCCCC | |
| of Ab27 | AAGCTGCTGATCTACGCCGCCAGCAGCAGGCAGAGCGGCGTGCC | |
| scFv | CAGCAGGTTCAGCGGCAGCGGCAGCGGCACCGACTTCACCCTGA | |
| | CCATCAGCAGCCTGCAGCCCGAGGACTTCGCCACCTACTACTGCC | |
| | AGCAGGTGTACGACACCCCCCTGACCTTCGGCTGCGGCACCAAGG | |
| | TGGAGATCAAGGGCGGCGGCGGCAGCGGCGGCGGCGGCAGCGGC | |
| | GGCGGCGGCAGCGGCGGCGGCGGCAGCGAGGTGCAGCTGGTGGA | |
| | GAGCGGCGGCGGCCTGGTGCAGCCCGGCGGCAGCCTGAGGCTGA | |
| | GCTGCGCCGCCAGCGGCTTCACCTTCAGCAGCTACTGGATGAGCT | |
| | GGGTGAGGCAGGCCCCCGGCAAGTGCCTGGAGTGGGTGGCCAAC | |
| | ATCAACCAGGACGGCAGCGAGGTGTACTACGTGGACAGCGTGAA | |
| | GGGCAGGTTCACCATCAGCAGGGACAACGCCAAGAACAGCCTGT | |
| | ACCTGCAGATGAACAGCCTGAGGGCCGAGGACACCGCCGTGTAC | |
| | TACTGCGCCAGGGACGTGGGCCCCGGCATCGCCTACCAGGGCCAC | |
| | TTCGACTACTGGGGCCAGGGCACCCTGGTGACCGTGAGCAGC | |
| | [SEQ ID NO: 521] | |
| | GAGGTGCAGCTGGTGGAGAGCGGCGGCGGCCTGGTGCAGCCCGG | |
| | CGGCAGCCTGAGGCTGAGCTGCGCCGCCAGCGGCTTCACCTTCAG | |
| | CAGCTACTGGATGAGCTGGGTGAGGCAGGCCCCCGGCAAGTGCC | |
| | TGGAGTGGGTGGCCAACATCAACCAGGACGGCAGCGAGGTGTAC | |
| | TACGTGGACAGCGTGAAGGGCAGGTTCACCATCAGCAGGGACAA | |
| | CGCCAAGAACAGCCTGTACCTGCAGATGAACAGCCTGAGGGCCG | |
| | AGGACACCGCCGTGTACTACTGCGCCAGGGACGTGGGCCCCGGC | |
| | ATCGCCTACCAGGGCCACTTCGACTACTGGGGCCAGGGCACCCTG | |
| | GTGACCGTGAGCAGCGGCGGCGGCGGCAGCGGCGGCGGCGGCAG | |
| | CGGCGGCGGCGGCAGCGGCGGCGGCGGCAGCGACATCCAGATGA | |
| | CCCAGAGCCCCAGCAGCCTGAGCGCCAGCGTGGGCGACAGGGTG | |
| | ACCATCACCTGCAGGGCCAGCCAGAGCATCTACTACTACCTGAAC | |
| | TGGTACCAGCAGAAGCCCGGCAAGGCCCCCAAGCTGCTGATCTAC | |
| | GCCGCCAGCAGCAGGCAGAGCGGCGTGCCCAGCAGGTTCAGCGG | |
| | CAGCGGCAGCGGCACCGACTTCACCCTGACCATCAGCAGCCTGCA | |
| | GCCCGAGGACTTCGCCACCTACTACTGCCAGCAGGTGTACGACAC | |
| | CCCCCTGACCTTCGGCTGCGGCACCAAGGTGGAGATCAAG [SEQ | |
| | ID NO: 522] | |
| ADI-11839 | QVQLVQSGAEVKKPGASVKVS | DIQMTQSPSSVSASVGDRVTITCE |
| [Ab28] | CKASGYTFSNYYMHWVRQAPG | ASKGISSWLAWYQQKPGKAPKL |
| | QGLEWMGWINPFSGGTRYAQK | LIYAASDLQSGVPSRFSGSGSGT |
| | FQGRVTMTRDTSTSTVYMELSS | DFTLTISSLQPEDFATYYCQQAFL |
| | LRSEDTAVYYCARDVGSSAYY | FPPTFGGGTKVEIK [SEQ ID |
| | YMDVWGKGTTVTVSS [SEQ ID | NO: 301] |
| | NO: 300] | CDR1: EASKGISSWLA [SEQ ID |
| | CDR1: NYYMH [SEQ ID NO: 406] | NO: 409] |
| | or YTFSNYYMH [SEQ ID | CDR2: AASDLQS [SEQ ID |
| | NO: 562](non-Kabat) | NO: 410] |
| | CDR2: WINPFSGGTRYAQKFQG | CDR3: QQAFLFPPT [SEQ ID |
| | [SEQ ID NO: 407] | NO: 411] |
| | CDR3: DVGSSAYYYMDV [SEQ | |
| | ID NO: 408] or | |
| | ARDVGSSAYYYMDV [SEQ ID | |
| | NO: 563](non-Kabat) | |
| scFv of | DIQMTQSPSSVSASVGDRVTITCEASKGISSWLAWYQQKPGKAPKLL | |
| Ab28 | IYAASDLQSGVPSRFSGSGSGTDFTLTISSLQPEDFATYYCQQAFLFPP | |
| | TFG [symbol] GTKVEIKGGGGSGGGGSGGGGSGGGGSQVQLVQSGAEVKK | |
| | PGASVKVSCKASGYTFSNYYMHWVRQAPGQ [symbol] LEWMGWINPFSGGT | |
| | RYAQKFQGRVTMTRDTSTSTVYMELSSLRSEDTAVYYCARDVGSSA | |
| | YYYMDVWGKGTTVTVSS [SEQ ID NO: 481] | |
| | QVQLVQSGAEVKKPGASVKVSCKASGYTFSNYYMHWVRQAPGQ [symbol] L | |
| | EWMGWINPFSGGTRYAQKFQGRVTMTRDTSTSTVYMELSSLRSEDT | |
| | AVYYCARDVGSSAYYYMDVWGKGTTVTVSSGGGGSGGGGSGGG | |
| | GSGGGGSDIQMTQSPSSVSASVGDRVTITCEASKGISSWLAWYQQK | |
| | PGKAPKLLIYAASDLQSGVPSRFSGSGSGTDFTLTISSLQPEDFATYYC | |
| | QQAFLFPPTFG [symbol] GTKVEIK [SEQ ID NO: 482] | |
| Exemplary | GACATCCAGATGACCCAGAGCCCCAGCAGCGTGAGCGCCAGCGT | |
| nucleotide | GGGCGACAGGGTGACCATCACCTGCGAGGCCAGCAAGGGCATCA | |
| sequence | GCAGCTGGCTGGCCTGGTACCAGCAGAAGCCCGGCAAGGCCCCC | |
| of Ab28 | AAGCTGCTGATCTACGCCGCCAGCGACCTGCAGAGCGGCGTGCCC | |
| scFv | AGCAGGTTCAGCGGCAGCGGCAGCGGCACCGACTTCACCCTGAC | |
| | CATCAGCAGCCTGCAGCCCGAGGACTTCGCCACCTACTACTGCCA | |
| | GCAGGCCTTCCTGTTCCCCCCCACCTTCGGCTGCGGCACCAAGGT | |
| | GGAGATCAAGGGCGGCGGCGGCAGCGGCGGCGGCGGCAGCGGC | |
| | GGCGGCGGCAGCGGCGGCGGCGGCAGCCAGGTGCAGCTGGTGCA | |
| | GAGCGGCGCCGAGGTGAAGAAGCCCGGCGCCAGCGTGAAGGTGA | |
| | GCTGCAAGGCCAGCGGCTACACCTTCAGCAACTACTACATGCACT | |
| | GGGTGAGGCAGGCCCCCGGCCAGTGCCTGGAGTGGATGGGCTGG | |
| | ATCAACCCCTTCAGCGGCGGCACCAGGTACGCCCAGAAGTTCCAG | |
| | GGCAGGGTGACCATGACCAGGGACACCAGCACCAGCACCGTGTA | |
| | CATGGAGCTGAGCAGCCTCAGGAGCGAGGACACCGCCGTGTACT | |
| | ACTGCGCCAGGGACGTGGGCAGCAGCGCCTACTACTACATGGAC | |
| | GTGTGGGGCAAGGGCACCACCGTGACCGTGAGCAGC [SEQ ID | |
| | NO: 523] | |
| | CAGGTGCAGCTGGTGCAGAGCGGCGCCGAGGTGAAGAAGCCCGG | |
| | CGCCAGCGTGAAGGTGAGCTGCAAGGCCAGCGGCTACACCTTCA | |
| | GCAACTACTACATGCACTGGGTGAGGCAGGCCCCCGGCCAGTGCC | |
| | TGGAGTGGATGGGCTGGATCAACCCCTTCAGCGGCGGCACCAGGT | |
| | ACGCCCAGAAGTTCCAGGGCAGGGTGACCATGACCAGGGACACC | |
| | AGCACCAGCACCGTGTACATGGAGCTGAGCAGCCTGAGGAGCGA | |
| | GGACACCGCCGTGTACTACTGCGCCAGGGACGTGGGCAGCAGCG | |
| | CCTACTACTACATGGACGTGTGGGGCAAGGGCACCACCGTGACCG | |
| | TGAGCAGCGGCGGCGGCGGCAGCGGCGGCGGCGGCAGCGGCGGC | |
| | GGCGGCAGCGGCGGCGGCGGCAGCGACATCCAGATGACCCAGAG | |
| | CCCCAGCAGCGTGAGCGCCAGCGTGGGCGACAGGGTGACCATCA | |
| | CCTGCGAGGCCAGCAAGGGCATCAGCAGCTGGCTGGCCTGGTAC | |
| | CAGCAGAAGCCCGGCAAGGCCCCCAAGCTGCTGATCTACGCCGC | |
| | CAGCGACCTGCAGAGCGGCGTGCCCAGCAGGTTCAGCGGCAGCG | |
| | GCAGCGGCACCGACTTCACCCTGACCATCAGCAGCCTGCAGCCCG | |
| | AGGACTTCGCCACCTACTACTGCCAGCAGGCCTTCCTGTTCCCCCC | |
| | CACCTTCGGCTGCGGCACCAAGGTGGAGATCAAG [SEQ ID | |
| | NO: 524] | |
| ADI-10152 | EVQLVQSGAEVKKPGESLKISC | EIVLTQSPGTLSLSPGERATLSCR |
| [Ab29] | KGSGYSFTSYWIGWVRQMPGK | ASQSVSSSFLAWYQQKPGQAPR |
| | GLEWMGSIYPGDSDTRYSPSFQ | LLIYGASSRATGIPDRFSGSGSGT |
| | GQVTISADKSISTAYLQWSSLKA | DFTLTISRLEPEDFAVYYCQQLDS |
| | SDTAMYYCARELAYGDYKGGV | PPPTFGGGTKVEIK [SEQ ID |
| | DYWGQGTLVTVSS [SEQ ID | NO: 303] |
| | NO: 302] | CDR1: RASQSVSSSFLA [SEQ ID |
| | CDR1: SYWIG [SEQ ID NO: 412] | NO: 415] |
| | or YSFTSYWIG [SEQ ID NO: 564] | CDR2: GASSRAT [SEQ ID NO: 416] |
| | (non-Kabat) | CDR3: QQLDSPPPT [SEQ ID |
| | CDR2: SIYPGDSDTRYSPSFQG | NO: 417] |
| | [SEQ ID NO: 413] | |
| | CDR3: ELAYGDYKGGVDY [SEQ | |
| | ID NO: 414] or | |
| | ARELAYGDYKGGVDY [SEQ ID | |
| | NO: 565](non-Kabat) | |
| scFv of | EIVLTQSPGTLSLSPGERATLSCRASQSVSSSFLAWYQQKPGQAPRLLI | |
| Ab29 | YGASSRATGIPDRFSGSGSGTDFTLTISRLEPEDFAVYYCQQLDSPPPT | |
| | FG [symbol] GTKVEIKGGGGSGGGGSGGGGSGGGGSEVQLVQSGAEVKKP | |
| | GESLKISCKGSGYSFTSYWIGWVRQMPGK [symbol] LEWMGSIYPGDSDTRY | |
| | SPSFQGQVTISADKSISTAYLQWSSLKASDTAMYYCARELAYGDYK | |
| | GGVDYWGQGTLVTVSS [SEQ ID NO: 483] | |
| | EVQLVQSGAEVKKPGESLKISCKGSGYSFTSYWIGWVRQMPGK [symbol] LE | |
| | WMGSIYPGDSDTRYSPSFQGQVTISADKSISTAYLQWSSLKASDTAM | |
| | YYCARELAYGDYKGGVDYWGQGTLVTVSSGGGGSGGGGSGGGG | |
| | SGGGGSEIVLTQSPGTLSLSPGERATLSCRASQSVSSSFLAWYQQKPG | |
| | QAPRLLIYGASSRATGIPDRFSGSGSGTDFTLTISRLEPEDFAVYYCQQ | |
| | LDSPPPTFG [symbol] GTKVEIK [SEQ ID NO: 484] | |
| Exemplary | GAGATCGTGCTGACCCAGAGCCCCGGCACCCTGAGCCTGAGCCCC | |
| nucleotide | GGCGAGAGGGCCACCCTGAGCTGCAGGGCCAGCCAGAGCGTGAG | |
| sequence | CAGCAGCTTCCTGGCCTGGTACCAGCAGAAGCCCGGCCAGGCCCC | |
| of Ab29 | CAGGCTGCTGATCTACGGCGCCAGCAGCAGGGCCACCGGCATCCC | |
| scFv | CGACAGGTTCAGCGGCAGCGGCAGCGGCACCGACTTCACCCTGA | |
| | CCATCAGCAGGCTGGAGCCCGAGGACTTCGCCGTGTACTACTGCC | |
| | AGCAGCTGGACAGCCCCCCCCCCACCTTCGGCTGCGGCACCAAGG | |
| | TGGAGATCAAGGGCGGCGGCGGCAGCGGCGGCGGCGGCAGCGGC | |
| | GGCGGCGGCAGCGGCGGCGGCGGCAGCGAGGTGCAGCTGGTGCA | |
| | GAGCGGCGCCGAGGTGAAGAAGCCCGGCGAGAGCCTGAAGATCA | |
| | GCTGCAAGGGCAGCGGCTACAGCTTCACCAGCTACTGGATCGGCT | |
| | GGGTGAGGCAGATGCCCGGCAAGTGCCTGGAGTGGATGGGCAGC | |
| | ATCTACCCCGGCGACAGCGACACCAGGTACAGCCCCAGCTTCCAG | |
| | GGCCAGGTGACCATCAGCGCCGACAAGAGCATCAGCACCGCCTA | |
| | CCTGCAGTGGAGCAGCCTGAAGGCCAGCGACACCGCCATGTACT | |
| | ACTGCGCCAGGGAGCTGGCCTACGGCGACTACAAGGGCGGCGTG | |
| | GACTACTGGGGCCAGGGCACCCTGGTGACCGTGAGCAGC [SEQ ID | |
| | NO: 525] | |
| | GAGGTGCAGCTGGTGCAGAGCGGCGCCGAGGTGAAGAAGCCCGG | |
| | CGAGAGCCTGAAGATCAGCTGCAAGGGCAGCGGCTACAGCTTCA | |
| | CCAGCTACTGGATCGGCTGGGTGAGGCAGATGCCCGGCAACTGC | |
| | CTGGAGTGGATGGGCAGCATCTACCCCGGCGACAGCGACACCAG | |
| | GTACAGCCCCAGCTTCCAGGGCCAGGTGACCATCAGCGCCGACA | |
| | AGAGCATCAGCACCGCCTACCTGCAGTGGAGCAGCCTGAAGGCC | |
| | AGCGACACCGCCATGTACTACTGCGCCAGGGAGCTGGCCTACGGC | |
| | GACTACAAGGGCGGCGTGGACTACTGGGGCCAGGGCACCCTGGT | |
| | GACCGTGAGCAGCGGCGGCGGCGGCAGCGGCGGCGGCGGCAGCG | |
| | GCGGCGGCGGCAGCGGCGGCGGCGGCAGCGAGATCGTGCTGACC | |
| | CAGAGCCCCGGCACCCTGAGCCTGAGCCCCGGCGAGAGGGCCAC | |
| | CCTGAGCTGCAGGGCCAGCCAGAGCGTGAGCAGCAGCTTCCTGG | |
| | CCTGGTACCAGCAGAAGCCCGGCCAGGCCCCCAGGCTGCTGATCT | |
| | ACGGCGCCAGCAGCAGGGCCACCGGCATCCCCGACAGGTTCAGC | |
| | GGCAGCGGCAGCGGCACCGACTTCACCCTGACCATCAGCAGGCT | |
| | GGAGCCCGAGGACTTCGCCGTGTACTACTGCCAGCAGCTGGACAG | |
| | CCCCCCCCCCACCTTCGGCTGCGGCACCAAGGTGGAGATCAAG | |
| | [SEQ ID NO: 526] | |
| Clone 280- | QVQLVQSGAEVKKPGSSVKVSC | DIQLTQSPSSLSASVGDRVTITCR |
| 31-01 | KASGGTFSDYAISWVRQAPGQG | ASQGISSVLAWYQQKPGKAPKL |
| (mut) of | LEWMGRIIPILGVADYAQKFQG | LIYDASSLESGVPSRFSGSGSGTD |
| WO201204 | RVTITADKSTRTAYMELSSLRSE | FTLTISSLQPEDFATYYCQQFDSSI |
| 5752 | DTAVYYCARNWADAFDIWGQG | TFGQGTKLEIK [SEQ ID NO: 419] |
| | TMVTVSS [SEQ ID NO: 418] | CDR1: RASQGISSVLA [SEQ ID |
| | CDR1: DYAIS [SEQ ID NO: 422] | NO: 425] |
| | CDR2: RIIPILGVADYAQKFQG | CDR2: DASSLES [SEQ ID NO: 426] |
| | [SEQ ID NO: 423] | CDR3: QQFDSSIT [SEQ ID |
| | CDR3: NWADAFDI [SEQ ID | NO: 427] |
| | NO: 424] | |
| scFv of | DIQLTQSPSSLSASVGDRVTITCRASQGISSVLAWYQQKPGKAPKLLI | |
| clone 280- | YDASSLESGVPSRFSGSGSGTDFTLTISSLQPEDFATYYCQQFDSSITF | |
| 31-01 | G [symbol] GTKLEIKGGGGSGGGGSGGGGSGGGGSQVQLVQSGAEVKKPG | |
| (mut) of | SSVKVSCKASGGTFSDYAISWVRQAPGQ [symbol] LEWMGRIIPILGVADYAQ | |
| WO201204 | KFQGRVTITADKSTRTAYMELSSLRSEDTAVYYCARNWADAFDIWG | |
| 5752 | QGTMVTVSS [SEQ ID NO: 485] | |
| | QVQLVQSGAEVKKPGSSVKVSCKASGGTFSDYAISWVRQAPGQ [symbol] LE | |
| | WMGRIIPILGVADYAQKFQGRVTITADKSTRTAYMELSSLRSEDTAV | |
| | YYCARNWADAFDIWGQGTMVTVSSGGGGSGGGGSGGGGSGGGG | |
| | SDIQLTQSPSSLSASVGDRVTITCRASQGISSVLAWYQQKPGKAPKLL | |
| | IYDASSLESGVPSRFSGSGSGTDFTLTISSLQPEDFATYYCQQFDSSITF | |
| | G [symbol] GTKLEIK [SEQ ID NO: 486] | |
| lintuzumab | QVQLVQSGAEVKKPGSSVKVSC | DIQMTQSPSSLSASVGDRVTITCR |
| | KASGYTFTDYNMHWVRQAPGQ | ASESVDNYGISFMNWFQQKPGK |
| | GLEWIGYIYPYNGGTGYNQKFK | APKLLIYAASNQGSGVPSRFSGS |
| | SKATITADESTNTAYMELSSLRS | GSGTDFTLTISSLQPDDFATYYCQ |
| | EDTAVYYCARGRPAMDYWGQ | QSKEVPWTFGQGTKVEIK [SEQ |
| | GTLVTVSS [SEQ ID NO: 420] | ID NO: 421] |
| | CDR1: DYNMH [SEQ ID NO: 428] | CDR1: RASESVDNYGISFMN |
| | CDR2: YIYPYNGGTGYNQKFKS | [SEQ ID NO: 431] |
| | [SEQ ID NO: 429] | CDR2: AASNQGS [SEQ ID |
| | CDR3: GRPAMDY [SEQ ID | NO: 432] |
| | NO: 430] | CDR3: QQSKEVPWT [SEQ ID |
| | | NO: 433] |

| | |
|---|---|
| scFv of | DIQMTQSPSSLSASVGDRVTITCRASESVDNYGISFMNWFQQKPGKA |
| lintuzumab | PKLLIYAASNQGSGVPSRFSGSGSGTDFTLTISSLQPDDFATYYCQQS |
| | KEVPWTFG [symbol] GTKVEIKGGGGSGGGGSGGGGSGGGGSQVQLVQSG |
| | AEVKKPGSSVKVSCKASGYTFTDYNMHWVRQAPGQ [symbol] LEWIGYIYP |
| | YNGGTGYNQKFKSKATITADESTNTAYMELSSLRSEDTAVYYCARG |
| | RPAMDYWGQGTLVTVSS [SEQ ID NO: 487] |
| | QVQLVQSGAEVKKPGSSVKVSCKASGYTFTDYNMHWVRQAPGQ [symbol] L |
| | EWIGYIYPYNGGTGYNQKFKSKATITADESTNTAYMELSSLRSEDTA |
| | VYYCARGRPAMDYWGQGTLVTVSSGGGGSGGGGSGGGGSGGGG |
| | SDIQMTQSPSSLSASVGDRVTITCRASESVDNYGISFMNAVFQQKPGK |
| | APKLLIYAASNQGSGVPSRFSGSGSGTDFTLTISSLQPDDFATYYCQQ |
| | SKEVPWTFG [symbol] GTKVEIK [SEQ ID NO: 488] |

An Antigen Binding Site that Binds an Epitope on an Extracellular Domain of Human CD33 and/or Cynomolgus/Rhesus (Cyno) CD33

In one aspect, the invention provides an antigen binding site including a heavy chain variable domain that binds an epitope on an extracellular domain of human CD33 and/or Cynomolgus/Rhesus (cyno) CD33.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVQPGGSLRLSCAASGFTFSSYGMSWVRQAPGKGLEWVANIKQDGS EKYYVDSVKGRFTISRDNAKNSLYLQMNSLRAEDTAVYYCAREGGPYYDSSGYFVY YGMDVWGQGTTVTVSS [SEQ ID NO:1]. In some embodiments, the antibody heavy chain variable domain is at least 95% identical to SEQ ID NO:1. In some embodiments, the heavy chain variable domain includes amino acid sequences FTFSSYGMS [SEQ ID NO:21] as the first complementarity-determining region 1 (“CDR1”), NIKQDGSEKYYVDSVKG [SEQ ID NO:22] as the second CDR (“CDR2”), and AREGGPYYDSSGYFVYYGMDV [SEQ ID NO:23] as the third CDR (“CDR3”) of SEQ ID NO:1. In some embodiments, the heavy chain variable domain includes amino acid sequences SYGMS [SEQ ID NO:434] as the first complementarity-determining region 1 (“CDR1”), NIKQDGSEKYYVDSVKG [SEQ ID NO:22] as the second CDR (“CDR2”), and EGGPYYDSSGYFVYYGMDV [SEQ ID NO:435] as the third CDR (“CDR3”) of SEQ ID NO:1. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:1 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., at least 91%, 92%, 93%, 94%, 95%, 96%, 97%, 98%, 99%, or 100%) identical to the amino acid sequence DIQMTQ SPSTLSASVGDRVTITCRASQSISSWLAWYQQKPGKAPKLLIYDAS SLESGV PSRFSGSGSGTEFTLTISSLQPDDFATYYCQQYESFPTFGGGTKVEIK [SEQ ID NO:2]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:2, which includes amino acid sequences RASQSISSWLA [SEQ ID NO:24] as CDR1, DASSLES [SEQ ID NO:25] as CDR2, and QQYESFPT [SEQ ID NO:26] as CDR3 of SEQ ID NO:2.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANIKQDG SEKYYVDSVKGRFTISRDNAKNSLYLQMNSLRAEDTAVYYCARPLNAGELDVWGQ GTMVTVSS [SEQ ID NO:3]. In some embodiments, the antibody heavy chain variable domain is at least 95% identical to SEQ ID NO:3. In some embodiments, the heavy chain variable domain incorporates amino acid sequences FTFSSYWMS [SEQ ID NO:27] as CDR1, NIKQDGSEKYYVDSVKG [SEQ ID NO:28] as CDR2, and ARPLNAGELDV [SEQ ID NO:29] as CDR3 of SEQ ID NO:3. In some embodiments, the heavy chain variable domain incorporates amino acid sequences SYWMS [SEQ ID NO:181] as CDR1, NIKQDGSEKYYVDSVKG [SEQ ID NO:28] as CDR2, and PLNAGELDV [SEQ ID NO:436] as CDR3 of SEQ ID NO:3. In certain embodiments, the antibody heavy chain variable domain, which includes an amino acid sequence at least 90% (e.g., at least 91%, 92%, 93%, 94%, 95%, 96%, 97%, 98%, 99%, or 100%) identical to the amino acid sequence of SEQ ID NO:3, is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQLESYPLTFGGGTKVEIK [SEQ ID NO:4]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:4, which includes amino acid sequences RASQSISSWLA [SEQ ID NO:30] as CDR1, EASSLES [SEQ ID NO:31] as CDR2, and QQLESYPLT [SEQ ID NO:32] as CDR3 of SEQ ID NO:4.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLLESGGGLVQPGGSLRLSCAASGFTFSKYTMSWVRQAPGKGLEWVSAIVGSGE STYFADSVKGRFTISRDNSKNTLYLQMNSLRAEDTAVYYCAREGGPYYDSSGYFVY YGMDVWGQGTTVTVSS [SEQ ID NO:5]. In some embodiments, the antibody heavy chain variable domain is at least 95% identical to SEQ ID NO:5. In some embodiments, the heavy chain variable domain incorporates amino acid sequences FTFSKYTMS [SEQ ID NO:33] as CDR1, AIVGSGESTYFADSVKG [SEQ ID NO:34] as CDR2, and AREGGPYYDSSGYFVYYGMDV [SEQ ID NO:35] as CDR3 of SEQ ID NO:5. In some embodiments, the heavy chain variable domain incorporates amino acid sequences KYTMS [SEQ ID NO:183] as CDR1, AIVGSGESTYFADSVKG [SEQ ID NO:34] as CDR2, and EGGPYYDSSGYFVYYGMDV [SEQ ID NO:184] as CDR3 of SEQ ID NO:5. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:5 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., at least 91%, 92%, 93%, 94%, 95%, 96%, 97%, 98%, 99%, or 100%) identical to the amino acid sequence DIQMTQ SPSTLSASVGDRVTITCRASQSISSWLAWYQQKPGKAPKLLIYKAS SLESGV PSRFSGSGSGTEFTLTISSLQPDDFATYYCQQYDDLPTFGGGTKVEIK [SEQ ID NO:6]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:6, which includes amino acid sequences RASQSISSWLA [SEQ ID NO:36] as CDR1, KASSLES [SEQ ID NO:37] or KASSLE [SEQ ID NO:185] as CDR2, and QQYDDLPT [SEQ ID NO:38] as CDR3 of SEQ ID NO:6.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of QVQLVQSGAEVKKPGASVKVSCKASGYTFSDYYMHWVRQAPGQGLEWMGMINPS WGSTSYAQKFQGRVTMTRDTSTSTVYMELSSLRSEDTAVYYCAREAADGFVGERYF DLWGRGTLVTVSS [SEQ ID NO:7]. In some embodiments, the antibody heavy chain variable domain is at least 95% identical to SEQ ID NO:7. In some embodiments, the heavy chain variable domain incorporates amino acid sequences YTFSDYYMH [SEQ ID NO:39] as CDR1, MINPSWGSTSYAQKFQG [SEQ ID NO:40] as CDR2, and AREAADGFVGERYFDL [SEQ ID NO:41] as CDR3 of SEQ ID NO:7. In some embodiments, the heavy chain variable domain incorporates amino acid sequences DYYMH [SEQ ID NO:437] as CDR1, AIVGSGESTYFADSVKG [SEQ ID NO:34] as CDR2, and EAADGFVGERYFDL [SEQ ID NO:438] as CDR3 of SEQ ID NO:7. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., at least 91%, 92%, 93%, 94%, 95%, 96%, 97%, 98%, 99%, or 100%) identical to the amino acid sequence DIVMTQSPLSLPVTPGEPASISCRSSQSLLYSNGYNYLDWYLQKPGQSPQLLIYLGSN RASGVPDRF SGSGSGTDFTLKISRVEAEDVGVYYCMQDVALPITFGGGTKVEIK [SEQ ID NO:8]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:8, which includes amino acid sequences RSSQSLLYSNGYNYLD [SEQ ID NO:42] as CDR1, LGSNRAS [SEQ ID NO:43] as CDR2, and MQDVALPIT [SEQ ID NO:44] as CDR3 of SEQ ID NO:8.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVQPGGSLRLSCAASGFTFGSYWMSWVRQAPGKGLEWVATIKQDG SEKSYVDSVKGRFTISRDNAKNSLYLQMNSLRAEDTAVYYCARPLNAGELDVWGQ GTMVTVSS [SEQ ID NO:9]. In some embodiments, the antibody heavy chain variable domain is at least 95% identical to SEQ ID NO:9. In some embodiments, the heavy chain variable domain incorporates amino acid sequences FTFGSYWMS [SEQ ID NO:45] as CDR1, TIKQDGSEKSYVDSVKG [SEQ ID NO:46] as CDR2, and ARPLNAGELDV [SEQ ID NO:47] as CDR3 of SEQ ID NO:9. In some embodiments, the heavy chain variable domain incorporates amino acid sequences SYWMS [SEQ ID NO:181] as CDR1, TIKQDGSEKSYVDSVKG [SEQ ID NO:46] as CDR2, and RPLNAGELDV [SEQ ID NO:182] as CDR3 of SEQ ID NO:9. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:9 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:10]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:10, which includes amino acid sequences RASQSISSWLA [SEQ ID NO:48] as CDR1, EASSLES [SEQ ID NO:49] as CDR2, and QQSQSYPPIT [SEQ ID NO:50] as CDR3 of SEQ ID NO:10.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVQPGGSLRLSCAASGFTFPSYWMSWVRQAPGKGLEWVATIKRDGS EKGYVD SVKGRFTISRDNAKNSLYLQMNSLRAEDTAVYYCARPLNAGELDVWGQG TMVTVSS [SEQ ID NO:11]. In some embodiments, the antibody heavy chain variable domain is at least 95% identical to SEQ ID NO:11. In some embodiments, the heavy chain variable domain incorporates amino acid sequences FTFPSYWMS [SEQ ID NO:51] as CDR1, TIKRDGSEKGYVDSVKG [SEQ ID NO:52] as CDR2, and ARPLNAGELDV [SEQ ID NO:53] as CDR3 of SEQ ID NO:11. In some embodiments, the heavy chain variable domain incorporates amino acid sequences SYWMS [SEQ ID NO:181] as CDR1, TIKRDGSEKGYVDSVKG [SEQ ID NO:52] as CDR2, and PLNAGELDV [SEQ ID NO:439] as CDR3 of SEQ ID NO:11. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:11 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:12]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:12, which includes amino acid sequences RASQSISSWLA [SEQ ID NO:54] as CDR1, EASSLES [SEQ ID NO:55] as CDR2, and QQSQSYPPIT [SEQ ID NO:56] as CDR3 of SEQ ID NO:12.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of QVQLVQSGAEVKKPGASVKVSCKASGYTFGTYYMHWVRQAPGQGLEWMGIINPSR GSTVYAQKFQGRVTMTRDTSTSTVYMELSSLRSEDTAVYYCARGAGYDDEDMDV WGKGTTVTVSS [SEQ ID NO:13]. In some embodiments, the antibody heavy chain variable domain is at least 95% identical to SEQ ID NO:13. In some embodiments, the heavy chain variable domain incorporates amino acid sequences YTFGTYYMH [SEQ ID NO:57] as CDR1, IINPSRGSTVYAQKFQG [SEQ ID NO:58] as CDR2, and ARGAGYDDEDMDV [SEQ ID NO:59] as CDR3 of SEQ ID NO:13. In some embodiments, the heavy chain variable domain incorporates amino acid sequences TYYMH [SEQ ID NO:440] as CDR1, TIKRDGSEKGYVDSVKG [SEQ ID NO:52] as CDR2, and GAGYDDEDMDV [SEQ ID NO:441] as CDR3 of SEQ ID NO:13. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:13 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSSVSASVGDRVTITCRASQGIDSWLAWYQQKPGKAPKLLIYAASSLQSG VPSRFSGSGSGTDFTLTISSLQPEDFATYYCQQAHSYPLTFGGGTKVEIK [SEQ ID NO:14]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:14, which includes amino acid sequences RASQGIDSWLA [SEQ ID NO:60] as CDR1, AASSLQS [SEQ ID NO:61] as CDR2, and QQAHSYPLT [SEQ ID NO:62] as CDR3 of SEQ ID NO:14.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVKPGGSLRLSCAASGFTFSSYAMSWVRQAPGKGLEWVSSISSSSEG IYYADSVKGRFTISRDNAKNSLYLQMNSLRAEDTAVYYCAREGGPYYDSSGYFVYY GMDVWGQGTTVTVSS [SEQ ID NO:15]. In some embodiments, the antibody heavy chain variable domain is at least 95% identical to SEQ ID NO:15. In some embodiments, the heavy chain variable domain incorporates amino acid sequences FTFSSYAMS [SEQ ID NO:63] as CDR1, SISSSSEGIYYADSVKG [SEQ ID NO:64] as CDR2, and AREGGPYYDSSGYFVYYGMDV [SEQ ID NO:65] as CDR3 of SEQ ID NO:15. In some embodiments, the heavy chain variable domain incorporates amino acid sequences SYAMS [SEQ ID NO:442] as CDR1, SISSSSEGIYYADSVKG [SEQ ID NO:64] as CDR2, and EGGPYYDSSGYFVYYGMDV [SEQ ID NO:443] as CDR3 of SEQ ID NO:15. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:15 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSTLSASVGDRVTITCRASNSISSWLAWYQQKPGKAPKLLIYEASSTKSGV PSRFSGSGSGTEFTLTISSLQPDDFATYYCQQYDDLPTFGGGTKVEIK [SEQ ID NO:16]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:16, which includes amino acid sequences RASNSISSWLA [SEQ ID NO:66] as CDR1, EASSTKS [SEQ ID NO:67] as CDR2, and QQYDDLPT [SEQ ID NO:68] as CDR3 of SEQ ID NO:16.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANINTDG SEVYYVDSVKGRFTISRDNAKNSLYLQMNSLRAEDTAVYYCARDVGPGIAYQGHFD YWGQGTLVTVSS [SEQ ID NO:17]. In some embodiments, the antibody heavy chain variable domain is at least 95% identical to SEQ ID NO:17. In some embodiments, the heavy chain variable domain incorporates amino acid sequences FTFSSYWMS [SEQ ID NO:69] as CDR1, NINTDGSEVYYVDSVKG [SEQ ID NO:70] as CDR2, and ARDVGPGIAYQGHFDY [SEQ ID NO:71] as CDR3 of SEQ ID NO:17. In some embodiments, the heavy chain variable domain incorporates amino acid sequences SYWMS [SEQ ID NO:181] as CDR1, NINTDGSEVYYVDSVKG [SEQ ID NO:70] as CDR2, and DVGPGIAYQGHFDY [SEQ ID NO:444] as CDR3 of SEQ ID NO:17. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:17 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., at least 91%, 92%, 93%, 94%, 95%, 96%, 97%, 98%, 99%, or 100%) identical to the amino acid sequence DIQMTQSPSSLSASVGDRVTITCRASQVIYSYLNWYQQKPGKAPKLLIYAASSLKSGV PSRFSGSGSGTDFTLTISSLQPEDFATYYCQQVYDTPLTFGGGTKVEIK [SEQ ID NO:18]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:18, which includes amino acid sequences RASQVIYSYLN [SEQ ID NO:72] as CDR1, AASSLKS [SEQ ID NO:73] as CDR2, and QQVYDTPLT [SEQ ID NO:74] as CDR3 of SEQ ID NO:18.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of QLQLQESGPGLVKPSETLSLTCTVSGGSISSTDYYWGWIRQPPGKGLEWIGSIGYSGT YYNPSLKSRVTISVDTSKNQFSLKLSSVTAADTAVYYCARETAHDVHGMDVWGQG TTVTVSS [SEQ ID NO:19]. In some embodiments, the antibody heavy chain variable domain is at least 95% identical to SEQ ID NO:19. In some embodiments, the heavy chain variable domain incorporates amino acid sequences GSISSTDYYWG [SEQ ID NO:75] as CDR1, SIGYSGTYYNPSLKS [SEQ ID NO:76] as CDR2, and ARETAHDVHGMDV [SEQ ID NO:77] as CDR3 of SEQ ID NO:19. In some embodiments, the heavy chain variable domain incorporates amino acid sequences STDYYWG [SEQ ID NO:445] as CDR1, SIGYSGTYYNPSLKS [SEQ ID NO:76] as CDR2, and ETAHDVHGMDV [SEQ ID NO:446] as CDR3 of SEQ ID NO:19. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:19 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., at least 91%, 92%, 93%, 94%, 95%, 96%, 97%, 98%, 99%, or 100%) identical to the amino acid sequence EIVLTQSPATLSLSPGERATLSCRASHSVYSYLAWYQQKPGQAPRLLIYDASNRATGI PARFSGSGSGTDFTLTISSLEPEDFAVYYCQQYDNLPTFGGGTKVEIK [SEQ ID NO:20]. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:20, which includes amino acid sequences RASHSVYSYLA [SEQ ID NO:78] as CDR1, DASNRAT [SEQ ID NO:79] as CDR2, and QQYDNLPT [SEQ ID NO:80] as CDR3 of SEQ ID NO:20.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:266. In some embodiments, the antibody heavy chain variable domain is at least 95% identical to SEQ ID NO:266. In some embodiments, the heavy chain variable domain incorporates amino acid sequences SEQ ID NO:304 as CDR1, SEQ ID NO:305 as CDR2, and SEQ ID NO:306 as CDR3 of SEQ ID NO:266. In some embodiments, the heavy chain variable domain incorporates amino acid sequences SEQ ID NO:528 as CDR1, SEQ ID NO:305 as CDR2, and SEQ ID NO:529 as CDR3 of SEQ ID NO:266. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:266 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267, which includes amino acid sequences SEQ ID NO:307 as CDR1, SEQ ID NO:308 as CDR2, and SEQ ID NO:309 as CDR3 of SEQ ID NO:267.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:268. In some embodiments, the antibody heavy chain variable domain is at least 95% identical to SEQ ID NO:268. In some embodiments, the heavy chain variable domain incorporates amino acid sequences SEQ ID NO:310 as CDR1, SEQ ID NO:311 as CDR2, and SEQ ID NO:312 as CDR3 of SEQ ID NO:268. In some embodiments, the heavy chain variable domain incorporates amino acid sequences SEQ ID NO:530 as CDR1, SEQ ID NO:311 as CDR2, and SEQ ID NO:531 as CDR3 of SEQ ID NO:268. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:268 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269, which includes amino acid sequences SEQ ID NO:313 as CDR1, SEQ ID NO:314 as CDR2, and SEQ ID NO:315 as CDR3 of SEQ ID NO:269.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SEQ ID NO:316 as CDR1, SEQ ID NO:317 as CDR2, and SEQ ID NO:318 as CDR3 of SEQ ID NO:270. In some embodiments, the heavy chain variable domain incorporates amino acid sequences SEQ ID NO:532 as CDR1, SEQ ID NO:317 as CDR2, and SEQ ID NO:533 as CDR3 of SEQ ID NO:270. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271, which includes amino acid sequences SEQ ID NO:319 as CDR1, SEQ ID NO:320 as CDR2, and SEQ ID NO:321 as CDR3 of SEQ ID NO:271.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences SEQ ID NO:322 as CDR1, SEQ ID NO:323 as CDR2, and SEQ ID NO:324 as CDR3 of SEQ ID NO:272. In some embodiments, the heavy chain variable domain incorporates amino acid sequences SEQ ID NO:534 as CDR1, SEQ ID NO:323 as CDR2, and SEQ ID NO:535 as CDR3 of SEQ ID NO:272. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273, which includes amino acid sequences SEQ ID NO:325 as CDR1, SEQ ID NO:326 as CDR2, and SEQ ID NO:327 as CDR3 of SEQ ID NO:273.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:274. In some embodiments, the antibody heavy chain variable domain is at least 95% identical to SEQ ID NO:274. In some embodiments, the heavy chain variable domain incorporates amino acid sequences SEQ ID NO:328 as CDR1, SEQ ID NO:329 as CDR2, and SEQ ID NO:330 as CDR3 of SEQ ID NO:274. In some embodiments, the heavy chain variable domain incorporates amino acid sequences SEQ ID NO:536 as CDR1, SEQ ID NO:329 as CDR2, and SEQ ID NO:537 as CDR3 of SEQ ID NO:274. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:274 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275, which includes amino acid sequences SEQ ID NO:331 as CDR1, SEQ ID NO:332 as CDR2, and SEQ ID NO:333 as CDR3 of SEQ ID NO:275.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:276. In some embodiments, the antibody heavy chain variable domain is at least 95% identical to SEQ ID NO:276. In some embodiments, the heavy chain variable domain incorporates amino acid sequences SEQ ID NO:334 as CDR1, SEQ ID NO:335 as CDR2, and SEQ ID NO:336 as CDR3 of SEQ ID NO:276. In some embodiments, the heavy chain variable domain incorporates amino acid sequences SEQ ID NO:538 as CDR1, SEQ ID NO:335 as CDR2, and SEQ ID NO:539 as CDR3 of SEQ ID NO:276. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:276 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277, which includes amino acid sequences SEQ ID NO:337 as CDR1, SEQ ID NO:338 as CDR2, and SEQ ID NO:339 as CDR3 of SEQ ID NO:277.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SEQ ID NO:340 as CDR1, SEQ ID NO:341 as CDR2, and SEQ ID NO:342 as CDR3 of SEQ ID NO:278. In some embodiments, the heavy chain variable domain incorporates amino acid sequences SEQ ID NO:540 as CDR1, SEQ ID NO:341 as CDR2, and SEQ ID NO:541 as CDR3 of SEQ ID NO:278. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279, which includes amino acid sequences SEQ ID NO:343 as CDR1, SEQ ID NO:344 as CDR2, and SEQ ID NO:345 as CDR3 of SEQ ID NO:279.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SEQ ID NO:346 as CDR1, SEQ ID NO:347 as CDR2, and SEQ ID NO:348 as CDR3 of SEQ ID NO:280. In some embodiments, the heavy chain variable domain incorporates amino acid sequences SEQ ID NO:542 as CDR1, SEQ ID NO:347 as CDR2, and SEQ ID NO:543 as CDR3 of SEQ ID NO:280. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281, which includes amino acid sequences SEQ ID NO:349 as CDR1, SEQ ID NO:350 as CDR2, and SEQ ID NO:351 as CDR3 of SEQ ID NO:281.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SEQ ID NO:352 as CDR1, SEQ ID NO:353 as CDR2, and SEQ ID NO:354 as CDR3 of SEQ ID NO:282. In some embodiments, the heavy chain variable domain incorporates amino acid sequences SEQ ID NO:544 as CDR1, SEQ ID NO:353 as CDR2, and SEQ ID NO:545 as CDR3 of SEQ ID NO:282. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283, which includes amino acid sequences SEQ ID NO:355 as CDR1, SEQ ID NO:356 as CDR2, and SEQ ID NO:357 as CDR3 of SEQ ID NO:283.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SEQ ID NO:358 as CDR1, SEQ ID NO:359 as CDR2, and SEQ ID NO:360 as CDR3 of SEQ ID NO:284. In some embodiments, the heavy chain variable domain incorporates amino acid sequences SEQ ID NO:546 as CDR1, SEQ ID NO:359 as CDR2, and SEQ ID NO:360 as CDR3 of SEQ ID NO:547. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285, which includes amino acid sequences SEQ ID NO:361 as CDR1, SEQ ID NO:362 as CDR2, and SEQ ID NO:363 as CDR3 of SEQ ID NO:285.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:286. In some embodiments, the antibody heavy chain variable domain is at least 95% identical to SEQ ID NO:286. In some embodiments, the heavy chain variable domain incorporates amino acid sequences SEQ ID NO:364 as CDR1, SEQ ID NO:365 as CDR2, and SEQ ID NO:366 as CDR3 of SEQ ID NO:286. In some embodiments, the heavy chain variable domain incorporates amino acid sequences SEQ ID NO:548 as CDR1, SEQ ID NO:365 as CDR2, and SEQ ID NO:549 as CDR3 of SEQ ID NO:286. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:286 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287, which includes amino acid sequences SEQ ID NO:367 as CDR1, SEQ ID NO:368 as CDR2, and SEQ ID NO:369 as CDR3 of SEQ ID NO:287.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:290. In some embodiments, the antibody heavy chain variable domain is at least 95% identical to SEQ ID NO:290. In some embodiments, the heavy chain variable domain incorporates amino acid sequences SEQ ID NO:376 as CDR1, SEQ ID NO:377 as CDR2, and SEQ ID NO:378 as CDR3 of SEQ ID NO:290. In some embodiments, the heavy chain variable domain incorporates amino acid sequences SEQ ID NO:552 as CDR1, SEQ ID NO:377 as CDR2, and SEQ ID NO:553 as CDR3 of SEQ ID NO:290. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:290 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291, which includes amino acid sequences SEQ ID NO:379 as CDR1, SEQ ID NO:380 as CDR2, and SEQ ID NO:381 as CDR3 of SEQ ID NO:291.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:292. In some embodiments, the antibody heavy chain variable domain is at least 95% identical to SEQ ID NO:292. In some embodiments, the heavy chain variable domain incorporates amino acid sequences SEQ ID NO:382 as CDR1, SEQ ID NO:383 as CDR2, and SEQ ID NO:384 as CDR3 of SEQ ID NO:292. In some embodiments, the heavy chain variable domain incorporates amino acid sequences SEQ ID NO:554 as CDR1, SEQ ID NO:383 as CDR2, and SEQ ID NO:555 as CDR3 of SEQ ID NO:292. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:292 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293, which includes amino acid sequences SEQ ID NO:385 as CDR1, SEQ ID NO:386 as CDR2, and SEQ ID NO:387 as CDR3 of SEQ ID NO:293.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:294. In some embodiments, the antibody heavy chain variable domain is at least 95% identical to SEQ ID NO:294. In some embodiments, the heavy chain variable domain incorporates amino acid sequences SEQ ID NO:388 as CDR1, SEQ ID NO:389 as CDR2, and SEQ ID NO:390 as CDR3 of SEQ ID NO:294. In some embodiments, the heavy chain variable domain incorporates amino acid sequences SEQ ID NO:556 as CDR1, SEQ ID NO:389 as CDR2, and SEQ ID NO:557 as CDR3 of SEQ ID NO:294. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:294 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295, which includes amino acid sequences SEQ ID NO:391 as CDR1, SEQ ID NO:392 as CDR2, and SEQ ID NO:393 as CDR3 of SEQ ID NO:295.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:296. In some embodiments, the antibody heavy chain variable domain is at least 95% identical to SEQ ID NO:296. In some embodiments, the heavy chain variable domain incorporates amino acid sequences SEQ ID NO:394 as CDR1, SEQ ID NO:395 as CDR2, and SEQ ID NO:396 as CDR3 of SEQ ID NO:296. In some embodiments, the heavy chain variable domain incorporates amino acid sequences SEQ ID NO:558 as CDR1, SEQ ID NO:395 as CDR2, and SEQ ID NO:559 as CDR3 of SEQ ID NO:296. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:296 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297, which includes amino acid sequences SEQ ID NO:397 as CDR1, SEQ ID NO:398 as CDR2, and SEQ ID NO:399 as CDR3 of SEQ ID NO:297.

In certain embodiments, the present invention provides an antigen binding site that binds an epitope on an extracellular domain of human CD33 and Cynomolgus/Rhesus (cyno) CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:298. In some embodiments, the antibody heavy chain variable domain is at least 95% identical to SEQ ID NO:298. In some embodiments, the heavy chain variable domain incorporates amino acid sequences SEQ ID NO:400 as CDR1, SEQ ID NO:401 as CDR2, and SEQ ID NO:402 as CDR3 of SEQ ID NO:298. In some embodiments, the heavy chain variable domain incorporates amino acid sequences SEQ ID NO:560 as CDR1, SEQ ID NO:401 as CDR2, and SEQ ID NO:561 as CDR3 of SEQ ID NO:298. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:298 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299, which includes amino acid sequences SEQ ID NO:403 as CDR1, SEQ ID NO:404 as CDR2, and SEQ ID NO:405 as CDR3 of SEQ ID NO:299.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:300. In some embodiments, the antibody heavy chain variable domain is at least 95% identical to SEQ ID NO:300. In some embodiments, the heavy chain variable domain incorporates amino acid sequences SEQ ID NO:406 as CDR1, SEQ ID NO:407 as CDR2, and SEQ ID NO:408 as CDR3 of SEQ ID NO:300. In some embodiments, the heavy chain variable domain incorporates amino acid sequences SEQ ID NO:562 as CDR1, SEQ ID NO:407 as CDR2, and SEQ ID NO:563 as CDR3 of SEQ ID NO:300. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:300 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301, which includes amino acid sequences SEQ ID NO:409 as CDR1, SEQ ID NO:410 as CDR2, and SEQ ID NO:411 as CDR3 of SEQ ID NO:301.

In certain embodiments, the present invention provides an antigen binding site that binds an epitope on an extracellular domain of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:302. In some embodiments, the antibody heavy chain variable domain is at least 95% identical to SEQ ID NO:302. In some embodiments, the heavy chain variable domain incorporates amino acid sequences SEQ ID NO:412 as CDR1, SEQ ID NO:413 as CDR2, and SEQ ID NO:414 as CDR3 of SEQ ID NO:302. In some embodiments, the heavy chain variable domain incorporates amino acid sequences SEQ ID NO:564 as CDR1, SEQ ID NO:413 as CDR2, and SEQ ID NO:565 as CDR3 of SEQ ID NO:302. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:302 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303, which includes amino acid sequences SEQ ID NO:415 as CDR1, SEQ ID NO:416 as CDR2, and SEQ ID NO:417 as CDR3 of SEQ ID NO:303.

An Antigen Binding Site that Recognizes and Binds a Conformational Epitope on an Extracellular Domain of the Human CD33 and/or the Cynomolgus/Rhesus (Cyno) CD33

In one aspect, the present invention provides an antigen binding site including a heavy chain variable domain that recognizes and binds one or more conformational epitopes on the extracellular domain of the human CD33 and/or the Cynomolgus/Rhesus (cyno) CD33.

In certain embodiments, the present invention provides an antigen binding site that recognizes and binds a conformational epitope partially located in the V domain of human CD33 extracellular domain; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANIKQDG SEKYYVDSVKGRF TISRDNAKNSLYLQMNSLRAEDTAVYYCARPLNAGELDVWGQ GTMVTVSS [SEQ ID NO:3]. In some embodiments, the antibody heavy chain variable domain is at least 95% identical to SEQ ID NO:3. In some embodiments, the heavy chain variable domain incorporates amino acid sequences FTFSSYWMS [SEQ ID NO:27] as CDR1, NIKQDGSEKYYVDSVKG [SEQ ID NO:28] as CDR2, and ARPLNAGELDV [SEQ ID NO:29] as CDR3 of SEQ ID NO:3. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:3 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 91%, 92%, 93%, 94%, 95%, 96%, 97%, 98%, 99%, or 100%) identical to the amino acid sequence DIQMTQ SP STLSASVGDRVTITCRASQ SIS SWLAWYQQKPGKAPKLLIYEAS SLESGV PSRFSGSGSGTEFTLTISSLQPDDFATYYCQQLESYPLTFGGGTKVEIK [SEQ ID NO:4]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 91%, 92%, 93%, 94%, 95%, 96%, 97%, 98%, 99%, or 100%) identical to the amino acid sequence of SEQ ID NO:4, which includes amino acid sequences RASQSISSWLA [SEQ ID NO:30] as CDR1, EASSLES [SEQ ID NO:31] as CDR2, and QQLESYPLT [SEQ ID NO:32] as CDR3 of SEQ ID NO:4.

In certain embodiments, the present invention provides an antigen binding site that recognizes and binds a conformational epitope partially located in the V domain of the human CD33 extracellular domain; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFPSYWMSWVRQAPGKGLEWVATIKRDGS EKGYVDSVKGRFTISRDNAKNSLYLQMNSLRAEDTAVYYCARPLNAGELDVWGQG TMVTVSS [SEQ ID NO:11]. In some embodiments, the antibody heavy chain variable domain is at least 95% identical to SEQ ID NO:11. In some embodiments, the heavy chain variable domain incorporates amino acid sequences FTFPSYWMS [SEQ ID NO:51] as CDR1, TIKRDGSEKGYVDSVKG [SEQ ID NO:52] as CDR2, and ARPLNAGELDV [SEQ ID NO:53] as CDR3 of SEQ ID NO:11. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:11 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:12]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 91%, 92%, 93%, 94%, 95%, 96%, 97%, 98%, 99%, or 100%) identical to the amino acid sequence of SEQ ID NO:12, which includes amino acid sequences RASQSISSWLA [SEQ ID NO:54] as CDR1, EASSLES [SEQ ID NO:55] as CDR2, and QQSQSYPPIT [SEQ ID NO:56] as CDR3 of SEQ ID NO:12.

In certain embodiments, the present invention provides an antigen binding site that recognizes and binds a conformational epitope partially located in the V domain of the human CD33 extracellular domain; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVKPGGSLRLSCAASGFTFSSYAMSWVRQAPGKGLEWVSSISSSSEG IYYAD SVKGRFTISRDNAKNSLYLQMNSLRAEDTAVYYCAREGGPYYDS SGYFVYY GMDVWGQGTTVTVSS [SEQ ID NO:15]. In some embodiments, the antibody heavy chain variable domain is at least 95% identical to SEQ ID NO:15. In some embodiments, the heavy chain variable domain incorporates amino acid sequences FTFSSYAMS [SEQ ID NO:63] as CDR1, SISSSSEGIYYADSVKG [SEQ ID NO:64] as CDR2, and AREGGPYYDSSGYFVYYGMDV [SEQ ID NO:65] as CDR3 of SEQ ID NO:15. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:15 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASNSISSWLAWYQQKPGKAPKLLIYEASSTKSGV PSRFSGSGSGTEFTLTISSLQPDDFATYYCQQYDDLPTFGGGTKVEIK [SEQ ID NO:16]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 91%, 92%, 93%, 94%, 95%, 96%, 97%, 98%, 99%, or 100%) identical to the amino acid sequence of SEQ ID NO:16, which includes amino acid sequences RASNSISSWLA [SEQ ID NO:66] as CDR1, EASSTKS [SEQ ID NO:67] as CDR2, and QQYDDLPT [SEQ ID NO:68] as CDR3 of SEQ ID NO:16.

In certain embodiments, the present invention provides an antigen binding site that recognizes and binds a conformational epitope partially located in the V domain of the human CD33 extracellular domain; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SEQ ID NO:316 as CDR1, SEQ ID NO:317 as CDR2, and SEQ ID NO:318 as CDR3 of SEQ ID NO:270. In some embodiments, the heavy chain variable domain incorporates amino acid sequences SEQ ID NO:532 as CDR1, SEQ ID NO:317 as CDR2, and SEQ ID NO:533 as CDR3 of SEQ ID NO:270. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271, which includes amino acid sequences SEQ ID NO:319 as CDR1, SEQ ID NO:320 as CDR2, and SEQ ID NO:321 as CDR3 of SEQ ID NO:271.

In certain embodiments, the present invention provides an antigen binding site that recognizes and binds a conformational epitope partially located in the V domain of the human CD33 extracellular domain; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences SEQ ID NO:322 as CDR1, SEQ ID NO:323 as CDR2, and SEQ ID NO:324 as CDR3 of SEQ ID NO:272. In some embodiments, the heavy chain variable domain incorporates amino acid sequences SEQ ID NO:534 as CDR1, SEQ ID NO:323 as CDR2, and SEQ ID NO:535 as CDR3 of SEQ ID NO:272. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273, which includes amino acid sequences SEQ ID NO:325 as CDR1, SEQ ID NO:326 as CDR2, and SEQ ID NO:327 as CDR3 of SEQ ID NO:273.

In certain embodiments, the present invention provides an antigen binding site that recognizes and binds a conformational epitope partially located in the V domain of the human CD33 extracellular domain; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SEQ ID NO:346 as CDR1, SEQ ID NO:347 as CDR2, and SEQ ID NO:348 as CDR3 of SEQ ID NO:280. In some embodiments, the heavy chain variable domain incorporates amino acid sequences SEQ ID NO:542 as CDR1, SEQ ID NO:347 as CDR2, and SEQ ID NO:543 as CDR3 of SEQ ID NO:280. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281, which includes amino acid sequences SEQ ID NO:349 as CDR1, SEQ ID NO:350 as CDR2, and SEQ ID NO:351 as CDR3 of SEQ ID NO:281.

An Antigen Binding Site that Recognizes and Binds a Conformational Epitope on an Extracellular Domain of the Human CD33 but not a Conformational Epitope on an Extracellular Domain of the Cynomolgus/Rhesus (Cyno) CD33

In one aspect, the present invention provides an antigen binding site that recognizes and binds one or more conformational epitopes on the extracellular domain of the human CD33 but does not recognize and/or bind one or more conformational epitopes on the extracellular domain of the cyno CD33.

In certain embodiments, the present invention provides an antigen binding site that recognizes and binds one or more conformational epitopes on the extracellular domain of the human CD33 but does not recognize and/or bind one or more conformational epitopes on the extracellular domain of the cyno CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTF SDYYMHW VRQAPGQGLEWMGMINP S WGSTSYAQKFQGRVTMTRDTSTSTVYMELS SLRSEDTAVYYCAREAADGFVGERYF DLWGRGTLVTVSS [SEQ ID NO:7]. In some embodiments, the antibody heavy chain variable domain is at least 95% identical to SEQ ID NO:7. In some embodiments, the heavy chain variable domain incorporates amino acid sequences YTFSDYYMH [SEQ ID NO:39] as CDR1, MINPSWGSTSYAQKFQG [SEQ ID NO:40] as CDR2, and AREAADGFVGERYFDL [SEQ ID NO:41] as CDR3 of SEQ ID NO:7. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence DIVMTQSPLSLPVTPGEPASISCRSSQSLLYSNGYNYLDWYLQKPGQSPQLLIYLGSN RASGVPDRF SGSGSGTDFTLKISRVEAEDVGVYYCMQDVALPITFGGGTKVEIK [SEQ ID NO:8]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence of SEQ ID NO:8, which includes amino acid sequences RSSQSLLYSNGYNYLD [SEQ ID NO:42] as CDR1, LGSNRAS [SEQ ID NO:43] as CDR2, and MQDVALPIT [SEQ ID NO:44] as CDR3 of SEQ ID NO:8.

In certain embodiments, the present invention provides an antigen binding site that recognizes and binds one or more conformational epitopes on the extracellular domain of the human CD33 but does not recognize and/or bind one or more conformational epitopes on the extracellular domain of the cyno CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTFGTYYMHWVRQAPGQGLEWMGIINPSR GSTVYAQKFQGRVTMTRDTSTSTVYMELSSLRSEDTAVYYCARGAGYDDEDMDV WGKGTTVTVSS [SEQ ID NO:13]. In some embodiments, the antibody heavy chain variable domain is at least 95% identical to SEQ ID NO:13. In some embodiments, the heavy chain variable domain incorporates amino acid sequences YTFGTYYMH [SEQ ID NO:57] as CDR1, IINPSRGSTVYAQKFQG [SEQ ID NO:58] as CDR2, and ARGAGYDDEDMDV [SEQ ID NO:59] as CDR3 of SEQ ID NO:13. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:13 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 91%, 92%, 93%, 94%, 95%, 96%, 97%, 98%, 99%, or 100%) identical to the amino acid sequence DIQMTQSPSSVSASVGDRVTITCRASQGIDSWLAWYQQKPGKAPKLLIYAASSLQSG VPSRFSGSGSGTDFTLTISSLQPEDFATYYCQQAHSYPLTFGGGTKVEIK [SEQ ID NO:14]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 91%, 92%, 93%, 94%, 95%, 96%, 97%, 98%, 99%, or 100%) identical to the amino acid sequence of SEQ ID NO:14, which includes amino acid sequences RASQGIDSWLA [SEQ ID NO:60] as CDR1, AASSLQS [SEQ ID NO:61] as CDR2, and QQAHSYPLT [SEQ ID NO:62] as CDR3 of SEQ ID NO:14.

In certain embodiments, an antibody binding site that includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTFGTYYMHWVRQAPGQGLEWMGIINPSR GSTVYAQKFQGRVTMTRDTSTSTVYMELS SLRSEDTAVYYCARGAGYDDEDMDV WGKGTTVTVSS [SEQ ID NO:13] binds to the full-length extracellular domain of human CD33, but does not bind human CD33 V domain or C domain individually, and does not cross-block binding to human CD33 with lintuzumab. In certain embodiments, an antibody binding site that includes a heavy chain variable domain including an amino acid sequence at least 90% (e.g., 91%, 92%, 93%, 94%, 95%, 96%, 97%, 98%, 99%, or 100%) identical to the amino acid sequence of SEQ ID NO:13, and is paired with an antibody light chain variable domain at least 90% (e.g., 91%, 92%, 93%, 94%, 95%, 96%, 97%, 98%, 99%, or 100%) identical to the amino acid sequence DIQMTQSPSSVSASVGDRVTITCRASQGIDSWLAWYQQKPGKAPKLLIYAASSLQSG VPSRFSGSGSGTDFTLTISSLQPEDFATYYCQQAHSYPLTFGGGTKVEIK [SEQ ID NO:14], binds to the full-length extracellular domain of human CD33, but does not bind human CD33 V domain or C domain individually, and does not cross-block binding to human CD33 with lintuzumab. In certain embodiments, an antibody binding site that includes a heavy chain variable domain including an amino acid sequence at least 90% (e.g., 91%, 92%, 93%, 94%, 95%, 96%, 97%, 98%, 99%, or 100%) identical to the amino acid sequence of SEQ ID NO:13, and is paired with an antibody light chain variable domain at least 90% (e.g., 91%, 92%, 93%, 94%, 95%, 96%, 97%, 98%, 99%, or 100%) identical to the amino acid sequence of SEQ ID NO:14, which includes amino acid sequences RASQGIDSWLA [SEQ ID NO:60] as CDR1, AASSLQS [SEQ ID NO:61] as CDR2, and QQAHSYPLT [SEQ ID NO:62] as CDR3 of SEQ ID NO:14, binds to the full-length extracellular domain of human CD33, but does not bind human CD33 V domain or C domain individually, and does not cross-block binding to human CD33 with lintuzumab.

An Antigen Binding Site that Binds to the R69G Allele of Human CD33

In one aspect, the present invention provides an antigen binding site that binds to the R69G allele of human CD33. In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFSSYGMSWVRQAPGKGLEWVANIKQDGS EKYYVDSVKGRFTISRDNAKNSLYLQMNSLRAEDTAVYYCAREGGPYYDSSGYFVY YGMDVWGQGTTVTVSS [SEQ ID NO:1]. In some embodiments, the antibody heavy chain variable domain is at least 95% identical to SEQ ID NO:1. In some embodiments, the heavy chain variable domain includes amino acid sequences FTFSSYGMS [SEQ ID NO:21] as the first complementarity-determining region 1 (“CDR1”), NIKQDGSEKYYVDSVKG [SEQ ID NO:22] as the second CDR (“CDR2”), and AREGGPYYDSSGYFVYYGMDV [SEQ ID NO:23] as the third CDR (“CDR3”) of SEQ ID NO:1. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:1 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., 91%, 92%, 93%, 94%, 95%, 96%, 97%, 98%, 99%, or 100%) identical to the amino acid sequence DIQMTQ SPSTLSASVGDRVTITCRASQSISSWLAWYQQKPGKAPKLLIYDAS SLESGV PSRFSGSGSGTEFTLTISSLQPDDFATYYCQQYESFPTFGGGTKVEIK [SEQ ID NO:2]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., 91%, 92%, 93%, 94%, 95%, 96%, 97%, 98%, 99%, or 100%) identical to the amino acid sequence of SEQ ID NO:2, which includes amino acid sequences RASQSISSWLA [SEQ ID NO:24] as CDR1, DASSLES [SEQ ID NO:25] as CDR2, and QQYESFPT [SEQ ID NO:26] as CDR3 of SEQ ID NO:2.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFS SYWMSWVRQAPGKGLEWVANIKQDG SEKYYVDSVKGRF TISRDNAKNSLYLQMNSLRAEDTAVYYCARPLNAGELDVWGQ GTMVTVSS [SEQ ID NO:3]. In some embodiments, the antibody heavy chain variable domain is at least 95% identical to SEQ ID NO:3. In some embodiments, the heavy chain variable domain incorporates amino acid sequences FTFSSYWMS [SEQ ID NO:27] as CDR1, NIKQDGSEKYYVDSVKG [SEQ ID NO:28] as CDR2, and ARPLNAGELDV [SEQ ID NO:29] as CDR3 of SEQ ID NO:3. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:3 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQLESYPLTFGGGTKVEIK [SEQ ID NO:4]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 91%, 92%, 93%, 94%, 95%, 96%, 97%, 98%, 99%, or 100%) identical to the amino acid sequence of SEQ ID NO:4, which includes amino acid sequences RASQSISSWLA [SEQ ID NO:30] as CDR1, EASSLES [SEQ ID NO:31] as CDR2, and QQLESYPLT [SEQ ID NO:32] as CDR3 of SEQ ID NO:4.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLLESGGGLVQPGGSLRLSCAASGFTFSKYTMSWVRQAPGKGLEWVSAIVGSGE STYFADSVKGRFTISRDNSKNTLYLQMNSLRAEDTAVYYCAREGGPYYDSSGYFVY YGMDVWGQGTTVTVSS [SEQ ID NO:5]. In some embodiments, the antibody heavy chain variable domain is at least 95% identical to SEQ ID NO:5. In some embodiments, the heavy chain variable domain incorporates amino acid sequences FTFSKYTMS [SEQ ID NO:33] as CDR1, AIVGSGESTYFADSVKG [SEQ ID NO:34] as CDR2, and AREGGPYYDSSGYFVYYGMDV [SEQ ID NO:35] as CDR3 of SEQ ID NO:5. In some embodiments, the heavy chain variable domain incorporates amino acid sequences KYTMS [SEQ ID NO:183] as CDR1, AIVGSGESTYFADSVKG [SEQ ID NO:34] as CDR2, and EGGPYYDSSGYFVYYGMDV [SEQ ID NO:184] as CDR3 of SEQ ID NO:5. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:5 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., 91%, 92%, 93%, 94%, 95%, 96%, 97%, 98%, 99%, or 100%) identical to the amino acid sequence DIQMTQ SPSTLSASVGDRVTITCRASQSISSWLAWYQQKPGKAPKLLIYKAS SLESGV PSRFSGSGSGTEFTLTISSLQPDDFATYYCQQYDDLPTFGGGTKVEIK [SEQ ID NO:6]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., 91%, 92%, 93%, 94%, 95%, 96%, 97%, 98%, 99%, or 100%) identical to the amino acid sequence of SEQ ID NO:6, which includes amino acid sequences RASQSISSWLA [SEQ ID NO:36] as CDR1, KASSLES [SEQ ID NO:37] or KASSLE [SEQ ID NO:185] as CDR2, and QQYDDLPT [SEQ ID NO:38] as CDR3 of SEQ ID NO:6.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFGSYWMSWVRQAPGKGLEWVATIKQDG SEKSYVDSVKGRFTISRDNAKNSLYLQMNSLRAEDTAVYYCARPLNAGELDVWGQ GTMVTVSS [SEQ ID NO:9]. In some embodiments, the antibody heavy chain variable domain is at least 95% identical to SEQ ID NO:9. In some embodiments, the heavy chain variable domain incorporates amino acid sequences FTFGSYWMS [SEQ ID NO:45] as CDR1, TIKQDGSEKSYVDSVKG [SEQ ID NO:46] as CDR2, and ARPLNAGELDV [SEQ ID NO:47] as CDR3 of SEQ ID NO:9. In some embodiments, the heavy chain variable domain incorporates amino acid sequences SYWMS [SEQ ID NO:181] as CDR1, TIKQDGSEKSYVDSVKG [SEQ ID NO:46] as CDR2, and RPLNAGELDV [SEQ ID NO:182] as CDR3 of SEQ ID NO:9. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:9 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:10]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:9 can be paired with an antibody light chain variable domain at least 90% (e.g., 91%, 92%, 93%, 94%, 95%, 96%, 97%, 98%, 99%, or 100%) identical to the amino acid sequence of SEQ ID NO:10, which includes amino acid sequences RASQSISSWLA [SEQ ID NO:48] as CDR1, EASSLES [SEQ ID NO:49] as CDR2, and QQSQSYPPIT [SEQ ID NO:50] as CDR3 of SEQ ID NO:10.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFPSYWMSWVRQAPGKGLEWVATIKRDGS EKGYVD SVKGRFTISRDNAKNSLYLQMNSLRAEDTAVYYCARPLNAGELDVWGQG TMVTVSS [SEQ ID NO:11]. In some embodiments, the antibody heavy chain variable domain is at least 95% identical to SEQ ID NO:11. In some embodiments, the heavy chain variable domain incorporates amino acid sequences FTFPSYWMS [SEQ ID NO:51] as CDR1, TIKRDGSEKGYVDSVKG [SEQ ID NO:52] as CDR2, and ARPLNAGELDV [SEQ ID NO:53] as CDR3 of SEQ ID NO:11. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:11 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASQSISSWLAWYQQKPGKAPKLLIYEASSLESGV PSRFSGSGSGTEFTLTISSLQPDDFATYYCQQSQSYPPITFGGGTKVEIK [SEQ ID NO:12]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:11 can be paired with an antibody light chain variable domain at least 90% (e.g., 91%, 92%, 93%, 94%, 95%, 96%, 97%, 98%, 99%, or 100%) identical to the amino acid sequence of SEQ ID NO:12, which includes amino acid sequences RASQSISSWLA [SEQ ID NO:54] as CDR1, EASSLES [SEQ ID NO:55] as CDR2, and QQSQSYPPIT [SEQ ID NO:56] as CDR3 of SEQ ID NO:12.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTFGTYYMHWVRQAPGQGLEWMGIINPSR GSTVYAQKFQGRVTMTRDTSTSTVYMELS SLRSEDTAVYYCARGAGYDDEDMDV WGKGTTVTVSS [SEQ ID NO:13]. In some embodiments, the antibody heavy chain variable domain is at least 95% identical to SEQ ID NO:13. In some embodiments, the heavy chain variable domain incorporates amino acid sequences YTFGTYYMH [SEQ ID NO:57] as CDR1, IINPSRGSTVYAQKFQG [SEQ ID NO:58] as CDR2, and ARGAGYDDEDMDV [SEQ ID NO:59] as CDR3 of SEQ ID NO:13. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:13 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 91%, 92%, 93%, 94%, 95%, 96%, 97%, 98%, 99%, or 100%) identical to the amino acid sequence DIQMTQ SP S SVSASVGDRVTITCRASQGIDSWLAWYQQKPGKAPKLLIYAAS SLQ SG VPSRFSGSGSGTDFTLTISSLQPEDFATYYCQQAHSYPLTFGGGTKVEIK [SEQ ID NO:14]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:13 can be paired with an antibody light chain variable domain at least 90% (e.g., 91%, 92%, 93%, 94%, 95%, 96%, 97%, 98%, 99%, or 100%) identical to the amino acid sequence of SEQ ID NO:14, which includes amino acid sequences RASQGIDSWLA [SEQ ID NO:60] as CDR1, AASSLQS [SEQ ID NO:61] as CDR2, and QQAHSYPLT [SEQ ID NO:62] as CDR3 of SEQ ID NO:14.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVKPGGSLRLSCAASGFTFSSYAMSWVRQAPGKGLEWVSSISSSSEG IYYADSVKGRFTISRDNAKNSLYLQMNSLRAEDTAVYYCAREGGPYYDSSGYFVYY GMDVWGQGTTVTVSS [SEQ ID NO:15]. In some embodiments, the antibody heavy chain variable domain is at least 95% identical to SEQ ID NO:15. In some embodiments, the heavy chain variable domain incorporates amino acid sequences FTFSSYAMS [SEQ ID NO:63] as CDR1, SISSSSEGIYYADSVKG [SEQ ID NO:64] as CDR2, and AREGGPYYDSSGYFVYYGMDV [SEQ ID NO:65] as CDR3 of SEQ ID NO:15. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:15 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 91%, 92%, 93%, 94%, 95%, 96%, 97%, 98%, 99%, or 100%) identical to the amino acid sequence DIQMTQSPSTLSASVGDRVTITCRASNSISSWLAWYQQKPGKAPKLLIYEASSTKSGV PSRFSGSGSGTEFTLTISSLQPDDFATYYCQQYDDLPTFGGGTKVEIK [SEQ ID NO:16]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:15 can be paired with an antibody light chain variable domain at least 90% (e.g., 91%, 92%, 93%, 94%, 95%, 96%, 97%, 98%, 99%, or 100%) identical to the amino acid sequence of SEQ ID NO:16, which includes amino acid sequences RASNSISSWLA [SEQ ID NO:66] as CDR1, EASSTKS [SEQ ID NO:67] as CDR2, and QQYDDLPT [SEQ ID NO:68] as CDR3 of SEQ ID NO:16.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of EVQLVESGGGLVQPGGSLRLSCAASGFTFSSYWMSWVRQAPGKGLEWVANINTDG SEVYYVDSVKGRFTISRDNAKNSLYLQMNSLRAEDTAVYYCARDVGPGIAYQGHFD YWGQGTLVTVSS [SEQ ID NO:17]. In some embodiments, the antibody heavy chain variable domain is at least 95% identical to SEQ ID NO:17. In some embodiments, the heavy chain variable domain incorporates amino acid sequences FTFSSYWMS [SEQ ID NO:69] as CDR1, NINTDGSEVYYVDSVKG [SEQ ID NO:70] as CDR2, and ARDVGPGIAYQGHFDY [SEQ ID NO:71] as CDR3 of SEQ ID NO:17. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:17 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., 91%, 92%, 93%, 94%, 95%, 96%, 97%, 98%, 99%, or 100%) identical to the amino acid sequence DIQMTQSPSSLSASVGDRVTITCRASQVIYSYLNWYQQKPGKAPKLLIYAASSLKSGV PSRFSGSGSGTDFTLTISSLQPEDFATYYCQQVYDTPLTFGGGTKVEIK [SEQ ID NO:18]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:17 can be paired with an antibody light chain variable domain at least 90% (e.g., 91%, 92%, 93%, 94%, 95%, 96%, 97%, 98%, 99%, or 100%) identical to the amino acid sequence of SEQ ID NO:18, which includes amino acid sequences RASQVIYSYLN [SEQ ID NO:72] as CDR1, AASSLKS [SEQ ID NO:73] as CDR2, and QQVYDTPLT [SEQ ID NO:74] as CDR3 of SEQ ID NO:18.

In certain embodiments, the present invention provides an antigen binding site that binds to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QLQLQESGPGLVKPSETLSLTCTVSGGSISSTDYYWGWIRQPPGKGLEWIGSIGYSGT YYNPSLKSRVTISVDTSKNQFSLKLSSVTAADTAVYYCARETAHDVHGMDVWGQG TTVTVSS [SEQ ID NO:19]. In some embodiments, the antibody heavy chain variable domain is at least 95% identical to SEQ ID NO:19. In some embodiments, the heavy chain variable domain incorporates amino acid sequences GSISSTDYYWG [SEQ ID NO:75] as CDR1, SIGYSGTYYNPSLKS [SEQ ID NO:76] as CDR2, and ARETAHDVHGMDV [SEQ ID NO:77] as CDR3 of SEQ ID NO:19. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:19 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., 91%, 92%, 93%, 94%, 95%, 96%, 97%, 98%, 99%, or 100%) identical to the amino acid sequence EIVLTQSPATLSLSPGERATLSCRASHSVYSYLAWYQQKPGQAPRLLIYDASNRATGI PARFSGSGSGTDFTLTISSLEPEDFAVYYCQQYDNLPTFGGGTKVEIK [SEQ ID NO:20]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:19 can be paired with an antibody light chain variable domain at least 90% (e.g., 91%, 92%, 93%, 94%, 95%, 96%, 97%, 98%, 99%, or 100%) identical to the amino acid sequence of SEQ ID NO:20, which includes amino acid sequences RASHSVYSYLA [SEQ ID NO:78] as CDR1, DASNRAT [SEQ ID NO:79] as CDR2, and QQYDNLPT [SEQ ID NO:80] as CDR3 of SEQ ID NO:20.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:266. In some embodiments, the antibody heavy chain variable domain is at least 95% identical to SEQ ID NO:266. In some embodiments, the heavy chain variable domain incorporates amino acid sequences SEQ ID NO:304 as CDR1, SEQ ID NO:305 as CDR2, and SEQ ID NO:306 as CDR3 of SEQ ID NO:266. In some embodiments, the heavy chain variable domain incorporates amino acid sequences SEQ ID NO:528 as CDR1, SEQ ID NO:305 as CDR2, and SEQ ID NO:529 as CDR3 of SEQ ID NO:266. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:266 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267, which includes amino acid sequences SEQ ID NO:307 as CDR1, SEQ ID NO:308 as CDR2, and SEQ ID NO:309 as CDR3 of SEQ ID NO:267.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:268. In some embodiments, the antibody heavy chain variable domain is at least 95% identical to SEQ ID NO:268. In some embodiments, the heavy chain variable domain incorporates amino acid sequences SEQ ID NO:310 as CDR1, SEQ ID NO:311 as CDR2, and SEQ ID NO:312 as CDR3 of SEQ ID NO:268. In some embodiments, the heavy chain variable domain incorporates amino acid sequences SEQ ID NO:530 as CDR1, SEQ ID NO:311 as CDR2, and SEQ ID NO:531 as CDR3 of SEQ ID NO:268. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:268 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269, which includes amino acid sequences SEQ ID NO:313 as CDR1, SEQ ID NO:314 as CDR2, and SEQ ID NO:315 as CDR3 of SEQ ID NO:269.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SEQ ID NO:316 as CDR1, SEQ ID NO:317 as CDR2, and SEQ ID NO:318 as CDR3 of SEQ ID NO:270. In some embodiments, the heavy chain variable domain incorporates amino acid sequences SEQ ID NO:532 as CDR1, SEQ ID NO:317 as CDR2, and SEQ ID NO:533 as CDR3 of SEQ ID NO:270. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271, which includes amino acid sequences SEQ ID NO:319 as CDR1, SEQ ID NO:320 as CDR2, and SEQ ID NO:321 as CDR3 of SEQ ID NO:271.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences SEQ ID NO:322 as CDR1, SEQ ID NO:323 as CDR2, and SEQ ID NO:324 as CDR3 of SEQ ID NO:272. In some embodiments, the heavy chain variable domain incorporates amino acid sequences SEQ ID NO:534 as CDR1, SEQ ID NO:323 as CDR2, and SEQ ID NO:535 as CDR3 of SEQ ID NO:272. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273, which includes amino acid sequences SEQ ID NO:325 as CDR1, SEQ ID NO:326 as CDR2, and SEQ ID NO:327 as CDR3 of SEQ ID NO:273.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:274. In some embodiments, the antibody heavy chain variable domain is at least 95% identical to SEQ ID NO:274. In some embodiments, the heavy chain variable domain incorporates amino acid sequences SEQ ID NO:328 as CDR1, SEQ ID NO:329 as CDR2, and SEQ ID NO:330 as CDR3 of SEQ ID NO:274. In some embodiments, the heavy chain variable domain incorporates amino acid sequences SEQ ID NO:536 as CDR1, SEQ ID NO:329 as CDR2, and SEQ ID NO:537 as CDR3 of SEQ ID NO:274. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:274 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275, which includes amino acid sequences SEQ ID NO:331 as CDR1, SEQ ID NO:332 as CDR2, and SEQ ID NO:333 as CDR3 of SEQ ID NO:275.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:276. In some embodiments, the antibody heavy chain variable domain is at least 95% identical to SEQ ID NO:276. In some embodiments, the heavy chain variable domain incorporates amino acid sequences SEQ ID NO:334 as CDR1, SEQ ID NO:335 as CDR2, and SEQ ID NO:336 as CDR3 of SEQ ID NO:276. In some embodiments, the heavy chain variable domain incorporates amino acid sequences SEQ ID NO:538 as CDR1, SEQ ID NO:335 as CDR2, and SEQ ID NO:539 as CDR3 of SEQ ID NO:276. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:276 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277, which includes amino acid sequences SEQ ID NO:337 as CDR1, SEQ ID NO:338 as CDR2, and SEQ ID NO:339 as CDR3 of SEQ ID NO:277.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SEQ ID NO:340 as CDR1, SEQ ID NO:341 as CDR2, and SEQ ID NO:342 as CDR3 of SEQ ID NO:278. In some embodiments, the heavy chain variable domain incorporates amino acid sequences SEQ ID NO:540 as CDR1, SEQ ID NO:341 as CDR2, and SEQ ID NO:541 as CDR3 of SEQ ID NO:278. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279, which includes amino acid sequences SEQ ID NO:343 as CDR1, SEQ ID NO:344 as CDR2, and SEQ ID NO:345 as CDR3 of SEQ ID NO:279.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SEQ ID NO:346 as CDR1, SEQ ID NO:347 as CDR2, and SEQ ID NO:348 as CDR3 of SEQ ID NO:280. In some embodiments, the heavy chain variable domain incorporates amino acid sequences SEQ ID NO:542 as CDR1, SEQ ID NO:347 as CDR2, and SEQ ID NO:543 as CDR3 of SEQ ID NO:280. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281, which includes amino acid sequences SEQ ID NO:349 as CDR1, SEQ ID NO:350 as CDR2, and SEQ ID NO:351 as CDR3 of SEQ ID NO:281.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SEQ ID NO:352 as CDR1, SEQ ID NO:353 as CDR2, and SEQ ID NO:354 as CDR3 of SEQ ID NO:282. In some embodiments, the heavy chain variable domain incorporates amino acid sequences SEQ ID NO:544 as CDR1, SEQ ID NO:353 as CDR2, and SEQ ID NO:545 as CDR3 of SEQ ID NO:282. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283, which includes amino acid sequences SEQ ID NO:355 as CDR1, SEQ ID NO:356 as CDR2, and SEQ ID NO:357 as CDR3 of SEQ ID NO:283.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SEQ ID NO:358 as CDR1, SEQ ID NO:359 as CDR2, and SEQ ID NO:360 as CDR3 of SEQ ID NO:284. In some embodiments, the heavy chain variable domain incorporates amino acid sequences SEQ ID NO:546 as CDR1, SEQ ID NO:359 as CDR2, and SEQ ID NO:360 as CDR3 of SEQ ID NO:547. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285, which includes amino acid sequences SEQ ID NO:361 as CDR1, SEQ ID NO:362 as CDR2, and SEQ ID NO:363 as CDR3 of SEQ ID NO:285.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:286. In some embodiments, the antibody heavy chain variable domain is at least 95% identical to SEQ ID NO:286. In some embodiments, the heavy chain variable domain incorporates amino acid sequences SEQ ID NO:364 as CDR1, SEQ ID NO:365 as CDR2, and SEQ ID NO:366 as CDR3 of SEQ ID NO:286. In some embodiments, the heavy chain variable domain incorporates amino acid sequences SEQ ID NO:548 as CDR1, SEQ ID NO:365 as CDR2, and SEQ ID NO:549 as CDR3 of SEQ ID NO:286. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:286 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287, which includes amino acid sequences SEQ ID NO:367 as CDR1, SEQ ID NO:368 as CDR2, and SEQ ID NO:369 as CDR3 of SEQ ID NO:287.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:292. In some embodiments, the antibody heavy chain variable domain is at least 95% identical to SEQ ID NO:292. In some embodiments, the heavy chain variable domain incorporates amino acid sequences SEQ ID NO:382 as CDR1, SEQ ID NO:383 as CDR2, and SEQ ID NO:384 as CDR3 of SEQ ID NO:292. In some embodiments, the heavy chain variable domain incorporates amino acid sequences SEQ ID NO:554 as CDR1, SEQ ID NO:383 as CDR2, and SEQ ID NO:555 as CDR3 of SEQ ID NO:292. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:292 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293, which includes amino acid sequences SEQ ID NO:385 as CDR1, SEQ ID NO:386 as CDR2, and SEQ ID NO:387 as CDR3 of SEQ ID NO:293.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:294. In some embodiments, the antibody heavy chain variable domain is at least 95% identical to SEQ ID NO:294. In some embodiments, the heavy chain variable domain incorporates amino acid sequences SEQ ID NO:388 as CDR1, SEQ ID NO:389 as CDR2, and SEQ ID NO:390 as CDR3 of SEQ ID NO:294. In some embodiments, the heavy chain variable domain incorporates amino acid sequences SEQ ID NO:556 as CDR1, SEQ ID NO:389 as CDR2, and SEQ ID NO:557 as CDR3 of SEQ ID NO:294. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:294 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295, which includes amino acid sequences SEQ ID NO:391 as CDR1, SEQ ID NO:392 as CDR2, and SEQ ID NO:393 as CDR3 of SEQ ID NO:295.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:296. In some embodiments, the antibody heavy chain variable domain is at least 95% identical to SEQ ID NO:296. In some embodiments, the heavy chain variable domain incorporates amino acid sequences SEQ ID NO:394 as CDR1, SEQ ID NO:395 as CDR2, and SEQ ID NO:396 as CDR3 of SEQ ID NO:296. In some embodiments, the heavy chain variable domain incorporates amino acid sequences SEQ ID NO:558 as CDR1, SEQ ID NO:395 as CDR2, and SEQ ID NO:559 as CDR3 of SEQ ID NO:296. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:296 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297, which includes amino acid sequences SEQ ID NO:397 as CDR1, SEQ ID NO:398 as CDR2, and SEQ ID NO:399 as CDR3 of SEQ ID NO:297.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:298. In some embodiments, the antibody heavy chain variable domain is at least 95% identical to SEQ ID NO:298. In some embodiments, the heavy chain variable domain incorporates amino acid sequences SEQ ID NO:400 as CDR1, SEQ ID NO:401 as CDR2, and SEQ ID NO:402 as CDR3 of SEQ ID NO:298. In some embodiments, the heavy chain variable domain incorporates amino acid sequences SEQ ID NO:560 as CDR1, SEQ ID NO:401 as CDR2, and SEQ ID NO:561 as CDR3 of SEQ ID NO:298. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:298 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299, which includes amino acid sequences SEQ ID NO:403 as CDR1, SEQ ID NO:404 as CDR2, and SEQ ID NO:405 as CDR3 of SEQ ID NO:299.

In certain embodiments, the present invention provides an antigen binding site that binds to the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:302. In some embodiments, the antibody heavy chain variable domain is at least 95% identical to SEQ ID NO:302. In some embodiments, the heavy chain variable domain incorporates amino acid sequences SEQ ID NO:412 as CDR1, SEQ ID NO:413 as CDR2, and SEQ ID NO:414 as CDR3 of SEQ ID NO:302. In some embodiments, the heavy chain variable domain incorporates amino acid sequences SEQ ID NO:564 as CDR1, SEQ ID NO:413 as CDR2, and SEQ ID NO:565 as CDR3 of SEQ ID NO:302. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:302 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303, which includes amino acid sequences SEQ ID NO:415 as CDR1, SEQ ID NO:416 as CDR2, and SEQ ID NO:417 as CDR3 of SEQ ID NO:303.

An Antigen Binding Site that does not Bind to the R69G Allele of Human CD33

In one aspect, the present invention provides an antigen binding site including a heavy chain variable domain that binds wild-type human CD33, but not the R69G allele of human CD33. In certain embodiments, the present invention provides an antigen binding site that does not bind to the R69G allele of human CD33; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTFSDYYMHWVRQAPGQGLEWMGMINPS WGSTSYAQKFQGRVTMTRDTSTSTVYMELSSLRSEDTAVYYCAREAADGFVGERYF DLWGRGTLVTVSS [SEQ ID NO:7]. In some embodiments, the antibody heavy chain variable domain is at least 95% identical to SEQ ID NO:7. In some embodiments, the heavy chain variable domain incorporates amino acid sequences YTFSDYYMH [SEQ ID NO:39] as CDR1, MINPSWGSTSYAQKFQG [SEQ ID NO:40] as CDR2, and AREAADGFVGERYFDL [SEQ ID NO:41] as CDR3 of SEQ ID NO:7. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence DIVMTQSPLSLPVTPGEPASISCRSSQSLLYSNGYNYLDWYLQKPGQSPQLLIYLGSN RASGVPDRFSGSGSGTDFTLKISRVEAEDVGVYYCMQDVALPITFGGGTKVEIK [SEQ ID NO:8]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence of SEQ ID NO:8, which includes amino acid sequences RSSQSLLYSNGYNYLD [SEQ ID NO:42] as CDR1, LGSNRAS [SEQ ID NO:43] as CDR2, and MQDVALPIT [SEQ ID NO:44] as CDR3 of SEQ ID NO:8.

In certain embodiments, the present invention provides an antigen binding site that binds to wild-type human CD33 but not the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

In certain embodiments, the present invention provides an antigen binding site that binds to wild-type human CD33 but not the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:290. In some embodiments, the antibody heavy chain variable domain is at least 95% identical to SEQ ID NO:290. In some embodiments, the heavy chain variable domain incorporates amino acid sequences SEQ ID NO:376 as CDR1, SEQ ID NO:377 as CDR2, and SEQ ID NO:378 as CDR3 of SEQ ID NO:290. In some embodiments, the heavy chain variable domain incorporates amino acid sequences SEQ ID NO:552 as CDR1, SEQ ID NO:377 as CDR2, and SEQ ID NO:553 as CDR3 of SEQ ID NO:290. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:290 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291, which includes amino acid sequences SEQ ID NO:379 as CDR1, SEQ ID NO:380 as CDR2, and SEQ ID NO:381 as CDR3 of SEQ ID NO:291.

In certain embodiments, the present invention provides an antigen binding site that binds to wild-type human CD33 but not the R69G allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:300. In some embodiments, the antibody heavy chain variable domain is at least 95% identical to SEQ ID NO:300. In some embodiments, the heavy chain variable domain incorporates amino acid sequences SEQ ID NO:406 as CDR1, SEQ ID NO:407 as CDR2, and SEQ ID NO:408 as CDR3 of SEQ ID NO:300. In some embodiments, the heavy chain variable domain incorporates amino acid sequences SEQ ID NO:562 as CDR1, SEQ ID NO:407 as CDR2, and SEQ ID NO:563 as CDR3 of SEQ ID NO:300. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:300 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301, which includes amino acid sequences SEQ ID NO:409 as CDR1, SEQ ID NO:410 as CDR2, and SEQ ID NO:411 as CDR3 of SEQ ID NO:301.

An Antigen Binding Site that Binds to a Unique Epitope Including R69 on Human CD33

In one aspect, the present invention provides an antigen binding site including a heavy chain variable domain that binds to a unique epitope on human CD33 that includes R69. In certain embodiments, the present invention provides an antigen binding site that binds to a unique epitope on human CD33 that includes R69; the antigen binding site includes a heavy chain variable domain including an amino acid sequence at least 90% (e.g., 91%, 92%, 93%, 94%, 95%, 96%, 97%, 98%, 99%, or 100%) identical to the amino acid sequence of QVQLVQSGAEVKKPGASVKVSCKASGYTFSDYYMHWVRQAPGQGLEWMGMINPS WGSTSYAQKFQGRVTMTRDTSTSTVYMELSSLRSEDTAVYYCAREAADGFVGERYF DLWGRGTLVTVSS [SEQ ID NO:7]. In some embodiments, the antibody heavy chain variable domain is at least 95% identical to SEQ ID NO:7. In some embodiments, the heavy chain variable domain incorporates amino acid sequences YTFSDYYMH [SEQ ID NO:39] as CDR1, MINPSWGSTSYAQKFQG [SEQ ID NO:40] as CDR2, and AREAADGFVGERYFDL [SEQ ID NO:41] as CDR3 of SEQ ID NO:7. In certain embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to CD33. For example, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence DIVMTQSPLSLPVTPGEPASISCRSSQSLLYSNGYNYLDWYLQKPGQSPQLLIYLGSN RASGVPDRFSGSGSGTDFTLKISRVEAEDVGVYYCMQDVALPITFGGGTKVEIK [SEQ ID NO:8]. In certain embodiments, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence of SEQ ID NO:8, which includes amino acid sequences RSSQSLLYSNGYNYLD [SEQ ID NO:42] as CDR1, LGSNRAS [SEQ ID NO:43] as CDR2, and MQDVALPIT [SEQ ID NO:44] as CDR3 of SEQ ID NO:8.

In certain embodiments, the present invention provides an antigen binding site that binds to a unique epitope on human CD33 that includes R69; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

In certain embodiments, the present invention provides an antigen binding site that binds to a unique epitope on human CD33 that includes R69; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:290. In some embodiments, the antibody heavy chain variable domain is at least 95% identical to SEQ ID NO:290. In some embodiments, the heavy chain variable domain incorporates amino acid sequences SEQ ID NO:376 as CDR1, SEQ ID NO:377 as CDR2, and SEQ ID NO:378 as CDR3 of SEQ ID NO:290. In some embodiments, the heavy chain variable domain incorporates amino acid sequences SEQ ID NO:552 as CDR1, SEQ ID NO:377 as CDR2, and SEQ ID NO:553 as CDR3 of SEQ ID NO:290. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:290 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291, which includes amino acid sequences SEQ ID NO:379 as CDR1, SEQ ID NO:380 as CDR2, and SEQ ID NO:381 as CDR3 of SEQ ID NO:291.

In certain embodiments, the present invention provides an antigen binding site that binds to a unique epitope on human CD33 that includes R69; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:300. In some embodiments, the antibody heavy chain variable domain is at least 95% identical to SEQ ID NO:300. In some embodiments, the heavy chain variable domain incorporates amino acid sequences SEQ ID NO:406 as CDR1, SEQ ID NO:407 as CDR2, and SEQ ID NO:408 as CDR3 of SEQ ID NO:300. In some embodiments, the heavy chain variable domain incorporates amino acid sequences SEQ ID NO:562 as CDR1, SEQ ID NO:407 as CDR2, and SEQ ID NO:563 as CDR3 of SEQ ID NO:300. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:300 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301, which includes amino acid sequences SEQ ID NO:409 as CDR1, SEQ ID NO:410 as CDR2, and SEQ ID NO:411 as CDR3 of SEQ ID NO:301.

An Antigen Binding Site that Binds to the S128N Allele of Human CD33

In one aspect, the present invention provides an antigen binding site that binds to the S128N allele of human CD33.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:268. In some embodiments, the antibody heavy chain variable domain is at least 95% identical to SEQ ID NO:268. In some embodiments, the heavy chain variable domain incorporates amino acid sequences SEQ ID NO:310 as CDR1, SEQ ID NO:311 as CDR2, and SEQ ID NO:312 as CDR3 of SEQ ID NO:268. In some embodiments, the heavy chain variable domain incorporates amino acid sequences SEQ ID NO:530 as CDR1, SEQ ID NO:311 as CDR2, and SEQ ID NO:531 as CDR3 of SEQ ID NO:268. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:268 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:268 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:269, which includes amino acid sequences SEQ ID NO:313 as CDR1, SEQ ID NO:314 as CDR2, and SEQ ID NO:315 as CDR3 of SEQ ID NO:269.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:274. In some embodiments, the antibody heavy chain variable domain is at least 95% identical to SEQ ID NO:274. In some embodiments, the heavy chain variable domain incorporates amino acid sequences SEQ ID NO:328 as CDR1, SEQ ID NO:329 as CDR2, and SEQ ID NO:330 as CDR3 of SEQ ID NO:274. In some embodiments, the heavy chain variable domain incorporates amino acid sequences SEQ ID NO:536 as CDR1, SEQ ID NO:329 as CDR2, and SEQ ID NO:537 as CDR3 of SEQ ID NO:274. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:274 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:274 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:275, which includes amino acid sequences SEQ ID NO:331 as CDR1, SEQ ID NO:332 as CDR2, and SEQ ID NO:333 as CDR3 of SEQ ID NO:275.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:276. In some embodiments, the antibody heavy chain variable domain is at least 95% identical to SEQ ID NO:276. In some embodiments, the heavy chain variable domain incorporates amino acid sequences SEQ ID NO:334 as CDR1, SEQ ID NO:335 as CDR2, and SEQ ID NO:336 as CDR3 of SEQ ID NO:276. In some embodiments, the heavy chain variable domain incorporates amino acid sequences SEQ ID NO:538 as CDR1, SEQ ID NO:335 as CDR2, and SEQ ID NO:539 as CDR3 of SEQ ID NO:276. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:276 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:276 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:277, which includes amino acid sequences SEQ ID NO:337 as CDR1, SEQ ID NO:338 as CDR2, and SEQ ID NO:339 as CDR3 of SEQ ID NO:277.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:292. In some embodiments, the antibody heavy chain variable domain is at least 95% identical to SEQ ID NO:292. In some embodiments, the heavy chain variable domain incorporates amino acid sequences SEQ ID NO:382 as CDR1, SEQ ID NO:383 as CDR2, and SEQ ID NO:384 as CDR3 of SEQ ID NO:292. In some embodiments, the heavy chain variable domain incorporates amino acid sequences SEQ ID NO:554 as CDR1, SEQ ID NO:383 as CDR2, and SEQ ID NO:555 as CDR3 of SEQ ID NO:292. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:292 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:292 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:293, which includes amino acid sequences SEQ ID NO:385 as CDR1, SEQ ID NO:386 as CDR2, and SEQ ID NO:387 as CDR3 of SEQ ID NO:293.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:294. In some embodiments, the antibody heavy chain variable domain is at least 95% identical to SEQ ID NO:294. In some embodiments, the heavy chain variable domain incorporates amino acid sequences SEQ ID NO:388 as CDR1, SEQ ID NO:389 as CDR2, and SEQ ID NO:390 as CDR3 of SEQ ID NO:294. In some embodiments, the heavy chain variable domain incorporates amino acid sequences SEQ ID NO:556 as CDR1, SEQ ID NO:389 as CDR2, and SEQ ID NO:557 as CDR3 of SEQ ID NO:294. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:294 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:294 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:295, which includes amino acid sequences SEQ ID NO:391 as CDR1, SEQ ID NO:392 as CDR2, and SEQ ID NO:393 as CDR3 of SEQ ID NO:295.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:296. In some embodiments, the antibody heavy chain variable domain is at least 95% identical to SEQ ID NO:296. In some embodiments, the heavy chain variable domain incorporates amino acid sequences SEQ ID NO:394 as CDR1, SEQ ID NO:395 as CDR2, and SEQ ID NO:396 as CDR3 of SEQ ID NO:296. In some embodiments, the heavy chain variable domain incorporates amino acid sequences SEQ ID NO:558 as CDR1, SEQ ID NO:395 as CDR2, and SEQ ID NO:559 as CDR3 of SEQ ID NO:296. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:296 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:296 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:297, which includes amino acid sequences SEQ ID NO:397 as CDR1, SEQ ID NO:398 as CDR2, and SEQ ID NO:399 as CDR3 of SEQ ID NO:297.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:298. In some embodiments, the antibody heavy chain variable domain is at least 95% identical to SEQ ID NO:298. In some embodiments, the heavy chain variable domain incorporates amino acid sequences SEQ ID NO:400 as CDR1, SEQ ID NO:401 as CDR2, and SEQ ID NO:402 as CDR3 of SEQ ID NO:298. In some embodiments, the heavy chain variable domain incorporates amino acid sequences SEQ ID NO:560 as CDR1, SEQ ID NO:401 as CDR2, and SEQ ID NO:561 as CDR3 of SEQ ID NO:298. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:298 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:298 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:299, which includes amino acid sequences SEQ ID NO:403 as CDR1, SEQ ID NO:404 as CDR2, and SEQ ID NO:405 as CDR3 of SEQ ID NO:299.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:290. In some embodiments, the antibody heavy chain variable domain is at least 95% identical to SEQ ID NO:290. In some embodiments, the heavy chain variable domain incorporates amino acid sequences SEQ ID NO:376 as CDR1, SEQ ID NO:377 as CDR2, and SEQ ID NO:378 as CDR3 of SEQ ID NO:290. In some embodiments, the heavy chain variable domain incorporates amino acid sequences SEQ ID NO:552 as CDR1, SEQ ID NO:377 as CDR2, and SEQ ID NO:553 as CDR3 of SEQ ID NO:290. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:290 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:290 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:291, which includes amino acid sequences SEQ ID NO:379 as CDR1, SEQ ID NO:380 as CDR2, and SEQ ID NO:381 as CDR3 of SEQ ID NO:291.

In certain embodiments, the present invention provides an antigen binding site that binds to the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:300. In some embodiments, the antibody heavy chain variable domain is at least 95% identical to SEQ ID NO:300. In some embodiments, the heavy chain variable domain incorporates amino acid sequences SEQ ID NO:406 as CDR1, SEQ ID NO:407 as CDR2, and SEQ ID NO:408 as CDR3 of SEQ ID NO:300. In some embodiments, the heavy chain variable domain incorporates amino acid sequences SEQ ID NO:562 as CDR1, SEQ ID NO:407 as CDR2, and SEQ ID NO:563 as CDR3 of SEQ ID NO:300. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:300 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:300 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:301, which includes amino acid sequences SEQ ID NO:409 as CDR1, SEQ ID NO:410 as CDR2, and SEQ ID NO:411 as CDR3 of SEQ ID NO:301.

An Antigen Binding Site that does not Bind to the S128N Allele of Human CD33

In one aspect, the present invention provides an antigen binding site including a heavy chain variable domain that binds to wild-type human CD33 but not the S128N allele of human CD33.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:302. In some embodiments, the antibody heavy chain variable domain is at least 95% identical to SEQ ID NO:302. In some embodiments, the heavy chain variable domain incorporates amino acid sequences SEQ ID NO:412 as CDR1, SEQ ID NO:413 as CDR2, and SEQ ID NO:414 as CDR3 of SEQ ID NO:302. In some embodiments, the heavy chain variable domain incorporates amino acid sequences SEQ ID NO:564 as CDR1, SEQ ID NO:413 as CDR2, and SEQ ID NO:565 as CDR3 of SEQ ID NO:302. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:302 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303, which includes amino acid sequences SEQ ID NO:415 as CDR1, SEQ ID NO:416 as CDR2, and SEQ ID NO:417 as CDR3 of SEQ ID NO:303.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:266. In some embodiments, the antibody heavy chain variable domain is at least 95% identical to SEQ ID NO:266. In some embodiments, the heavy chain variable domain incorporates amino acid sequences SEQ ID NO:304 as CDR1, SEQ ID NO:305 as CDR2, and SEQ ID NO:306 as CDR3 of SEQ ID NO:266. In some embodiments, the heavy chain variable domain incorporates amino acid sequences SEQ ID NO:528 as CDR1, SEQ ID NO:305 as CDR2, and SEQ ID NO:529 as CDR3 of SEQ ID NO:266. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:266 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267, which includes amino acid sequences SEQ ID NO:307 as CDR1, SEQ ID NO:308 as CDR2, and SEQ ID NO:309 as CDR3 of SEQ ID NO:267.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SEQ ID NO:316 as CDR1, SEQ ID NO:317 as CDR2, and SEQ ID NO:318 as CDR3 of SEQ ID NO:270. In some embodiments, the heavy chain variable domain incorporates amino acid sequences SEQ ID NO:532 as CDR1, SEQ ID NO:317 as CDR2, and SEQ ID NO:533 as CDR3 of SEQ ID NO:270. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271, which includes amino acid sequences SEQ ID NO:319 as CDR1, SEQ ID NO:320 as CDR2, and SEQ ID NO:321 as CDR3 of SEQ ID NO:271.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences SEQ ID NO:322 as CDR1, SEQ ID NO:323 as CDR2, and SEQ ID NO:324 as CDR3 of SEQ ID NO:272. In some embodiments, the heavy chain variable domain incorporates amino acid sequences SEQ ID NO:534 as CDR1, SEQ ID NO:323 as CDR2, and SEQ ID NO:535 as CDR3 of SEQ ID NO:272. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273, which includes amino acid sequences SEQ ID NO:325 as CDR1, SEQ ID NO:326 as CDR2, and SEQ ID NO:327 as CDR3 of SEQ ID NO:273.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SEQ ID NO:340 as CDR1, SEQ ID NO:341 as CDR2, and SEQ ID NO:342 as CDR3 of SEQ ID NO:278. In some embodiments, the heavy chain variable domain incorporates amino acid sequences SEQ ID NO:540 as CDR1, SEQ ID NO:341 as CDR2, and SEQ ID NO:541 as CDR3 of SEQ ID NO:278. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279, which includes amino acid sequences SEQ ID NO:343 as CDR1, SEQ ID NO:344 as CDR2, and SEQ ID NO:345 as CDR3 of SEQ ID NO:279.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SEQ ID NO:346 as CDR1, SEQ ID NO:347 as CDR2, and SEQ ID NO:348 as CDR3 of SEQ ID NO:280. In some embodiments, the heavy chain variable domain incorporates amino acid sequences SEQ ID NO:542 as CDR1, SEQ ID NO:347 as CDR2, and SEQ ID NO:543 as CDR3 of SEQ ID NO:280. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281, which includes amino acid sequences SEQ ID NO:349 as CDR1, SEQ ID NO:350 as CDR2, and SEQ ID NO:351 as CDR3 of SEQ ID NO:281.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SEQ ID NO:352 as CDR1, SEQ ID NO:353 as CDR2, and SEQ ID NO:354 as CDR3 of SEQ ID NO:282. In some embodiments, the heavy chain variable domain incorporates amino acid sequences SEQ ID NO:544 as CDR1, SEQ ID NO:353 as CDR2, and SEQ ID NO:545 as CDR3 of SEQ ID NO:282. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283, which includes amino acid sequences SEQ ID NO:355 as CDR1, SEQ ID NO:356 as CDR2, and SEQ ID NO:357 as CDR3 of SEQ ID NO:283.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SEQ ID NO:358 as CDR1, SEQ ID NO:359 as CDR2, and SEQ ID NO:360 as CDR3 of SEQ ID NO:284. In some embodiments, the heavy chain variable domain incorporates amino acid sequences SEQ ID NO:546 as CDR1, SEQ ID NO:359 as CDR2, and SEQ ID NO:360 as CDR3 of SEQ ID NO:547. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285, which includes amino acid sequences SEQ ID NO:361 as CDR1, SEQ ID NO:362 as CDR2, and SEQ ID NO:363 as CDR3 of SEQ ID NO:285.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:286. In some embodiments, the antibody heavy chain variable domain is at least 95% identical to SEQ ID NO:286. In some embodiments, the heavy chain variable domain incorporates amino acid sequences SEQ ID NO:364 as CDR1, SEQ ID NO:365 as CDR2, and SEQ ID NO:366 as CDR3 of SEQ ID NO:286. In some embodiments, the heavy chain variable domain incorporates amino acid sequences SEQ ID NO:548 as CDR1, SEQ ID NO:365 as CDR2, and SEQ ID NO:549 as CDR3 of SEQ ID NO:286. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:286 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287, which includes amino acid sequences SEQ ID NO:367 as CDR1, SEQ ID NO:368 as CDR2, and SEQ ID NO:369 as CDR3 of SEQ ID NO:287.

In certain embodiments, the present invention provides an antigen binding site that binds to wild-type human CD33 but not the S128N allele of human CD33; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

An Antigen Binding Site that Binds to a Unique Epitope Including S128 on Human CD33

In one aspect, the present invention provides an antigen binding site including a heavy chain variable domain that binds to a unique epitope on human CD33 that includes S128.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:302. In some embodiments, the antibody heavy chain variable domain is at least 95% identical to SEQ ID NO:302. In some embodiments, the heavy chain variable domain incorporates amino acid sequences SEQ ID NO:412 as CDR1, SEQ ID NO:413 as CDR2, and SEQ ID NO:414 as CDR3 of SEQ ID NO:302. In some embodiments, the heavy chain variable domain incorporates amino acid sequences SEQ ID NO:564 as CDR1, SEQ ID NO:413 as CDR2, and SEQ ID NO:565 as CDR3 of SEQ ID NO:302. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:302 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:302 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:303, which includes amino acid sequences SEQ ID NO:415 as CDR1, SEQ ID NO:416 as CDR2, and SEQ ID NO:417 as CDR3 of SEQ ID NO:303.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:266. In some embodiments, the antibody heavy chain variable domain is at least 95% identical to SEQ ID NO:266. In some embodiments, the heavy chain variable domain incorporates amino acid sequences SEQ ID NO:304 as CDR1, SEQ ID NO:305 as CDR2, and SEQ ID NO:306 as CDR3 of SEQ ID NO:266. In some embodiments, the heavy chain variable domain incorporates amino acid sequences SEQ ID NO:528 as CDR1, SEQ ID NO:305 as CDR2, and SEQ ID NO:529 as CDR3 of SEQ ID NO:266. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:266 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:266 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:267, which includes amino acid sequences SEQ ID NO:307 as CDR1, SEQ ID NO:308 as CDR2, and SEQ ID NO:309 as CDR3 of SEQ ID NO:267.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:270. In some embodiments, the antibody heavy chain variable domain is at least 95% identical to SEQ ID NO:270. In some embodiments, the heavy chain variable domain incorporates amino acid sequences SEQ ID NO:316 as CDR1, SEQ ID NO:317 as CDR2, and SEQ ID NO:318 as CDR3 of SEQ ID NO:270. In some embodiments, the heavy chain variable domain incorporates amino acid sequences SEQ ID NO:532 as CDR1, SEQ ID NO:317 as CDR2, and SEQ ID NO:533 as CDR3 of SEQ ID NO:270. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:270 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:270 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:271, which includes amino acid sequences SEQ ID NO:319 as CDR1, SEQ ID NO:320 as CDR2, and SEQ ID NO:321 as CDR3 of SEQ ID NO:271.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:272. In some embodiments, the antibody heavy chain variable domain is at least 95% identical to SEQ ID NO:272. In some embodiments, the heavy chain variable domain incorporates amino acid sequences SEQ ID NO:322 as CDR1, SEQ ID NO:323 as CDR2, and SEQ ID NO:324 as CDR3 of SEQ ID NO:272. In some embodiments, the heavy chain variable domain incorporates amino acid sequences SEQ ID NO:534 as CDR1, SEQ ID NO:323 as CDR2, and SEQ ID NO:535 as CDR3 of SEQ ID NO:272. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:272 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:272 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:273, which includes amino acid sequences SEQ ID NO:325 as CDR1, SEQ ID NO:326 as CDR2, and SEQ ID NO:327 as CDR3 of SEQ ID NO:273.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SEQ ID NO:340 as CDR1, SEQ ID NO:341 as CDR2, and SEQ ID NO:342 as CDR3 of SEQ ID NO:278. In some embodiments, the heavy chain variable domain incorporates amino acid sequences SEQ ID NO:540 as CDR1, SEQ ID NO:341 as CDR2, and SEQ ID NO:541 as CDR3 of SEQ ID NO:278. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279, which includes amino acid sequences SEQ ID NO:343 as CDR1, SEQ ID NO:344 as CDR2, and SEQ ID NO:345 as CDR3 of SEQ ID NO:279.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:280. In some embodiments, the antibody heavy chain variable domain is at least 95% identical to SEQ ID NO:280. In some embodiments, the heavy chain variable domain incorporates amino acid sequences SEQ ID NO:346 as CDR1, SEQ ID NO:347 as CDR2, and SEQ ID NO:348 as CDR3 of SEQ ID NO:280. In some embodiments, the heavy chain variable domain incorporates amino acid sequences SEQ ID NO:542 as CDR1, SEQ ID NO:347 as CDR2, and SEQ ID NO:543 as CDR3 of SEQ ID NO:280. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:280 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:280 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:281, which includes amino acid sequences SEQ ID NO:349 as CDR1, SEQ ID NO:350 as CDR2, and SEQ ID NO:351 as CDR3 of SEQ ID NO:281.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SEQ ID NO:352 as CDR1, SEQ ID NO:353 as CDR2, and SEQ ID NO:354 as CDR3 of SEQ ID NO:282. In some embodiments, the heavy chain variable domain incorporates amino acid sequences SEQ ID NO:544 as CDR1, SEQ ID NO:353 as CDR2, and SEQ ID NO:545 as CDR3 of SEQ ID NO:282. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283, which includes amino acid sequences SEQ ID NO:355 as CDR1, SEQ ID NO:356 as CDR2, and SEQ ID NO:357 as CDR3 of SEQ ID NO:283.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SEQ ID NO:358 as CDR1, SEQ ID NO:359 as CDR2, and SEQ ID NO:360 as CDR3 of SEQ ID NO:284. In some embodiments, the heavy chain variable domain incorporates amino acid sequences SEQ ID NO:546 as CDR1, SEQ ID NO:359 as CDR2, and SEQ ID NO:360 as CDR3 of SEQ ID NO:547. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285, which includes amino acid sequences SEQ ID NO:361 as CDR1, SEQ ID NO:362 as CDR2, and SEQ ID NO:363 as CDR3 of SEQ ID NO:285.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:286. In some embodiments, the antibody heavy chain variable domain is at least 95% identical to SEQ ID NO:286. In some embodiments, the heavy chain variable domain incorporates amino acid sequences SEQ ID NO:364 as CDR1, SEQ ID NO:365 as CDR2, and SEQ ID NO:366 as CDR3 of SEQ ID NO:286. In some embodiments, the heavy chain variable domain incorporates amino acid sequences SEQ ID NO:548 as CDR1, SEQ ID NO:365 as CDR2, and SEQ ID NO:549 as CDR3 of SEQ ID NO:286. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:286 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:286 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:287, which includes amino acid sequences SEQ ID NO:367 as CDR1, SEQ ID NO:368 as CDR2, and SEQ ID NO:369 as CDR3 of SEQ ID NO:287.

In certain embodiments, the present invention provides an antigen binding site that binds to a unique epitope on human CD33 that includes S128; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

### An Antigen Binding Site Binds to the V Domain of Human CD33 in a Glycosylation Sensitive Manner

In one aspect, the present invention provides an antigen binding site including a heavy chain variable domain that binds to the V domain of human CD33 in a glycosylation sensitive manner, e.g., binds to the V domain of CD33 only when the V domain is deglycosylated.

In certain embodiments, the present invention provides an antigen binding site that binds an epitope in the V domain of human CD33 only when the V domain is deglycosylated; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:278. In some embodiments, the antibody heavy chain variable domain is at least 95% identical to SEQ ID NO:278. In some embodiments, the heavy chain variable domain incorporates amino acid sequences SEQ ID NO:340 as CDR1, SEQ ID NO:341 as CDR2, and SEQ ID NO:342 as CDR3 of SEQ ID NO:278. In some embodiments, the heavy chain variable domain incorporates amino acid sequences SEQ ID NO:540 as CDR1, SEQ ID NO:341 as CDR2, and SEQ ID NO:541 as CDR3 of SEQ ID NO:278. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:278 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:278 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:279, which includes amino acid sequences SEQ ID NO:343 as CDR1, SEQ ID NO:344 as CDR2, and SEQ ID NO:345 as CDR3 of SEQ ID NO:279.

In certain embodiments, the present invention provides an antigen binding site that binds an epitope in the V domain of human CD33 only when the V domain is deglycosylated; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:282. In some embodiments, the antibody heavy chain variable domain is at least 95% identical to SEQ ID NO:282. In some embodiments, the heavy chain variable domain incorporates amino acid sequences SEQ ID NO:352 as CDR1, SEQ ID NO:353 as CDR2, and SEQ ID NO:354 as CDR3 of SEQ ID NO:282. In some embodiments, the heavy chain variable domain incorporates amino acid sequences SEQ ID NO:544 as CDR1, SEQ ID NO:353 as CDR2, and SEQ ID NO:545 as CDR3 of SEQ ID NO:282. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:282 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:282 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:283, which includes amino acid sequences SEQ ID NO:355 as CDR1, SEQ ID NO:356 as CDR2, and SEQ ID NO:357 as CDR3 of SEQ ID NO:283.

In certain embodiments, the present invention provides an antigen binding site that binds an epitope in the V domain of human CD33 only when the V domain is deglycosylated; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:284. In some embodiments, the antibody heavy chain variable domain is at least 95% identical to SEQ ID NO:284. In some embodiments, the heavy chain variable domain incorporates amino acid sequences SEQ ID NO:358 as CDR1, SEQ ID NO:359 as CDR2, and SEQ ID NO:360 as CDR3 of SEQ ID NO:284. In some embodiments, the heavy chain variable domain incorporates amino acid sequences SEQ ID NO:546 as CDR1, SEQ ID NO:359 as CDR2, and SEQ ID NO:360 as CDR3 of SEQ ID NO:547. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:284 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:284 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:285, which includes amino acid sequences SEQ ID NO:361 as CDR1, SEQ ID NO:362 as CDR2, and SEQ ID NO:363 as CDR3 of SEQ ID NO:285.

In certain embodiments, the present invention provides an antigen binding site that binds an epitope in the V domain of human CD33 only when the V domain is deglycosylated; the antigen binding site includes a heavy chain variable domain including an amino acid sequence at least 90% (e.g., at least 91%, 92%, 93%, 94%, 95%, 96%, 97%, 98%, 99%, or 100%) identical to the amino acid sequence of SEQ ID NO:288. In some embodiments, the antibody heavy chain variable domain is at least 95% identical to SEQ ID NO:288. In some embodiments, the heavy chain variable domain incorporates amino acid sequences SEQ ID NO:370 as CDR1, SEQ ID NO:371 as CDR2, and SEQ ID NO:372 as CDR3 of SEQ ID NO:288. In some embodiments, the heavy chain variable domain incorporates amino acid sequences SEQ ID NO:550 as CDR1, SEQ ID NO:371 as CDR2, and SEQ ID NO:551 as CDR3 of SEQ ID NO:288. In certain embodiments, the antibody heavy chain variable domain which includes an amino acid sequence at least 90% (e.g., at least 91%, 92%, 93%, 94%, 95%, 96%, 97%, 98%, 99%, or 100%) identical to the amino acid sequence of SEQ ID NO:288 is combined with a light chain variable domain to form an antigen-binding site capable of binding to CD33. For example, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289. In certain embodiments, an antibody heavy chain variable domain at least 90% (e.g., at least 91%, 92%, 93%, 94%, 95%, 96%, 97%, 98%, 99%, or 100%) identical to the amino acid sequence of SEQ ID NO:288 can be paired with an antibody light chain variable domain at least 90% (e.g., at least 91%, 92%, 93%, 94%, 95%, 96%, 97%, 98%, 99%, or 100%) identical to the amino acid sequence of SEQ ID NO:289, which includes amino acid sequences SEQ ID NO:373 as CDR1, SEQ ID NO:374 as CDR2, and SEQ ID NO:375 as CDR3 of SEQ ID NO:289.

An Antigen Binding Site that Binds to the Extracellular Domain in Human CD33 and/or Cyno CD33, Irrespective of the Glycosylation Profile of the Targeted CD33

In one aspect, the present invention provides an antigen binding site including a heavy chain variable domain which includes an amino acid sequence at least 90% (e.g., 90%, 91%, 92%, 93%, 94%, 95%, 96%, 97%, 98%, 99%, or 100%) identical to the amino acid sequence of SEQ ID NO: 1, 3, 5, 7, 9, 11, 13, 15, 17, 19, 266, 268, 270, 272, 274, 276, 278, 280, 282, 284, 286, 288, 290, 292, 294, 296, 298, 300, or 302, which binds to the extracellular domain in human CD33 irrespective of the glycosylation profile of the targeted CD33.

In certain embodiments, the present invention provides an antigen binding site including a heavy chain variable domain that binds to the extracellular domain in human CD33 and/or cyno CD33, such that the epitopes are unique compared to the epitopes targeted by one or more known anti-CD33 antibodies in the art. In certain embodiments, the present invention provides an antigen binding site including a heavy chain variable domain that binds to the extracellular domain in human CD33 and/or cyno CD33, which shows human or Cynomolgus/Rhesus (cyno) CD33 cross reactivity and high affinity binding to the target CD33.

A Second Antigen Binding Site Same or Different from the Antigen-Binding Site that Binds Human CD33

In certain embodiments, the present invention provides a protein that includes a human CD33 antigen-binding site including a heavy chain variable domain, which includes an amino acid sequence at least 90% (e.g., 90%, 91%, 92%, 93%, 94%, 95%, 96%, 97%, 98%, 99%, or 100%) identical to the amino acid sequence of SEQ ID NO: 1, 3, 5, 7, 9, 11, 13, 15, 17, 19, 266, 268, 270, 272, 274, 276, 278, 280, 282, 284, 286, 288, 290, 292, 294, 296, 298, 300, or 302, and further comprises a second antigen binding site same or different from the antigen-binding site that binds to human CD33.

Proteins with Antigen-Binding Sites

An antibody heavy chain variable domain of SEQ ID NOs: 1, 3, 5, 7, 9, 11, 13, 15, 17, 19, 266, 268, 270, 272, 274, 276, 278, 280, 282, 284, 286, 288, 290, 292, 294, 296, 298, 300, and/or 302 can optionally be coupled to an amino acid sequence at least 90% (e.g., 90%, 91%, 92%, 93%, 94%, 95%, 96%, 97%, 98%, 99%, or 100%) identical to an antibody constant region, such as an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, an IgG2 constant region, IgG3 constant region, or IgG4 constant region. In some other embodiments, the amino acid sequence of the constant region is at least 90% (e.g., 90%, 91%, 92%, 93%, 94%, 95%, 96%, 97%, 98%, 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

### II. Multi-Specific Binding Proteins

In certain embodiments, the present invention provides an antigen-binding site in a protein (e.g., a multi-specific binding protein) that binds to CD33 on a cancer cell, and the NKG2D receptor and CD16 receptor on natural killer cells to activate the natural killer cell. As used herein, the term “antibody” encompasses proteins (e.g., multi-specific binding proteins) that comprise one or more antigen-binding sites (e.g., an antigen-binding site that binds CD33), and is not limited to single-specific antibodies. In certain embodiments, the protein (e.g., multi-specific binding protein) or antibody is a trispecific antibody, also called Trispecific NK cell Engagement Therapy (TriNKET). The protein (e.g., a multi-specific binding protein) is useful in the pharmaceutical compositions and therapeutic methods described herein. Binding of the protein including an antigen-binding site that binds to CD33, and to NKG2D receptor and CD16 receptor on natural killer cell enhances the activity of the natural killer cell toward destruction of a cancer cell. Binding of the protein including an antigen-binding site that binds to CD33 (e.g., a multi-specific binding protein) on a cancer cell brings the cancer cell into proximity to the natural killer cell, which facilitates direct and indirect destruction of the cancer cell by the natural killer cell. Further description of exemplary multi-specific binding proteins is provided below.

In certain embodiments of the present disclosure, the first component of the multi-specific binding proteins binds to CD33-expressing cells, which can include but are not limited to AML, myelodysplastic syndromes, chronic myelomonocytic leukemia, myeloid blast crisis of chronic myeloid leukemia, and ALLs.

In certain embodiments of the present disclosure, the second component of the multi-specific binding proteins binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NKG2D receptors.

In certain embodiments of the present disclosure, the third component for the multi-specific binding proteins binds to cells expressing CD16, an Fc receptor on the surface of leukocytes including natural killer cells, macrophages, neutrophils, eosinophils, mast cells, and follicular dendritic cells.

Another aspect of the present invention provides a protein comprising an antigen-binding site that binds NKG2D, the antigen-binding site comprising a heavy chain variable domain comprising:

CDR1 comprising the amino acid sequence of SYSMN [SEQ ID NO:192];

CDR2 comprising the amino acid sequence of SISSSSSYIYYADSVKG [SEQ ID NO:112]; and

CDR3 comprising the amino acid sequence of GAPXGAAAGWFDP [SEQ ID NO:527], wherein X is A, V, L, I, P, F, W, G, S, T, C, N, Q, or Y; and a light chain variable domain comprising:

CDR1 comprising the amino acid sequence of RASQGISSWLA [SEQ ID NO:114],

CDR2 comprising the amino acid sequence of AASSLQS [SEQ ID NO:115], and

CDR3 comprising the amino acid sequence of QQGVSFPRT [SEQ ID NO:116].

In certain embodiments, X is A, V, L, I, P, F, or W. In certain embodiments, X is V, L, or I. In certain embodiments, the amino acid sequence of CDR3 in the heavy chain variable domain comprises the sequence of SEQ ID NO:195.

In certain embodiments, the antigen-binding site comprises a heavy chain variable domain comprising an amino acid sequence at least 90% (e.g., 91%, 92%, 93%, 94%, 95%, 96%, 97%, 98%, or 99%) identical to the amino acid sequence of SEQ ID NO:191; and a light chain variable domain comprising an amino acid sequence at least 90% (e.g., 91%, 92%, 93%, 94%, 95%, 96%, 97%, 98%, or 99%) identical to the amino acid sequence of SEQ ID NO:81. In certain embodiments, the antigen-binding site comprises a heavy chain variable domain comprising the amino acid sequence of SEQ ID NO:191; and a light chain variable domain comprising the amino acid sequence of SEQ ID NO:81.

In certain embodiments, the antigen-binding site that binds NKG2D is in the form of an Fab fragment. In certain embodiments, the antigen-binding site that binds NKG2D is in the form of an scFv.

In certain embodiments, the present invention provides a protein comprising (a) a first antigen-binding site comprising an Fab fragment that binds NKG2D as disclosed herein; (b) a second antigen-binding site comprising a single-chain variable fragment (scFv) that binds a tumor associated antigen (e.g., CD33); and (c) an antibody Fc domain or a portion thereof sufficient to bind CD16, or a third antigen-binding site that binds CD16.

The multi-specific binding proteins described herein can take various formats. For example, one format is a heterodimeric, multi-specific antibody which includes a first immunoglobulin heavy chain, a first immunoglobulin light chain, a second immunoglobulin heavy chain and a second immunoglobulin light chain. The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds CD33. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. The second immunoglobulin light chain, together with the second immunoglobulin heavy chain, forms an antigen-binding site that binds NKG2D. The first Fc domain and second Fc domain together are able to bind to CD16.

Another exemplary format involves a heterodimeric, multi-specific antibody which includes a first immunoglobulin heavy chain, a second immunoglobulin heavy chain and an immunoglobulin light chain. The first immunoglobulin heavy chain includes a first Fc (hinge-CH2-CH3) domain fused via either a linker or an antibody hinge to a single-chain variable fragment (scFv) composed of a heavy variable domain and light chain variable domain which pair and bind CD33 or NKG2D. The second immunoglobulin heavy chain includes a second Fc (hinge-CH2-CH3) domain, a second heavy chain variable domain and optionally a CH1 heavy chain domain. The immunoglobulin light chain includes a light chain variable domain and a constant light chain domain. The second immunoglobulin heavy chain pairs with the immunoglobulin light chain and binds to NKG2D or CD33. The first Fc domain and the second Fc domain together are able to bind to CD16.

One or more additional binding motifs may be fused to the C-terminus of the constant region CH3 domain, optionally via a linker sequence. In certain embodiments, the antigen-binding site could be a single-chain or disulfide-stabilized variable region (scFv) or could form a tetravalent or trivalent molecule.

In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcgammaRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab, without any changes being made to the other Fab.

In some embodiments, the multi-specific binding protein is in the 2-in-1 Ig format. In some embodiments, the multi-specific binding protein is in the ES form, which is a heterodimeric construct containing two different Fabs binding to targets 1 and target 2 fused to the Fc. Heterodimerization is ensured by electrostatic steering mutations in the Fc.

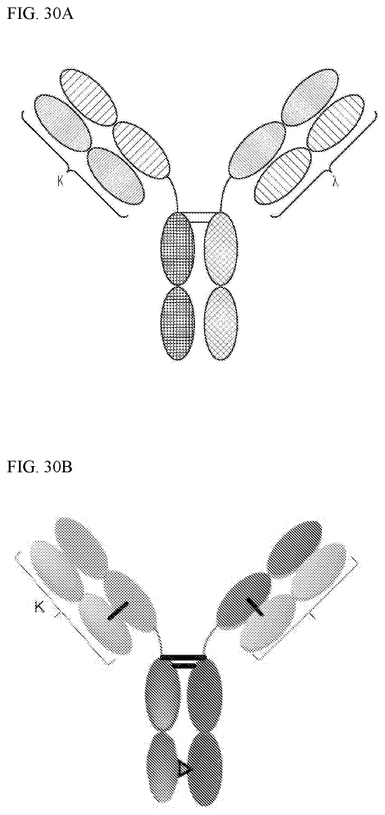

In some embodiments, the multi-specific binding protein is in the κλ-Body form, which is an heterodimeric constructs with two different Fabs fused to Fc stabilized by heterodimerization mutations: Fab1 targeting antigen 1 contains kappa LC, while second Fab targeting antigen 2 contains lambda LC. FIG. 30A is an exemplary representation of one form of a κλ-Body; FIG. 30B is an exemplary representation of another κλKk-Body.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies). In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)). In some embodiments, the multi-specific binding protein is in the LuZ-Y form, in which a leucine zipper is used to induce heterodimerization of two different HCs. (Wranik, B J. et al., J. Biol. Chem. (2012), 287:43331-9).

In some embodiments, the multi-specific binding protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab binding to target 1, and scFab binding to target 2 fused to Fc. Heterodimerization is ensured by mutations in the Fc.

In some embodiments, the multi-specific binding protein is in a DuetMab form, which is an heterodimeric construct containing two different Fabs binding to antigens 1 and 2, and Fc stabilized by heterodimerization mutations. Fab 1 and 2 contain differential S-S bridges that ensure correct LC and HC pairing.

In some embodiments, the multi-specific binding protein is in a CrossmAb form, which is an heterodimeric construct with two different Fabs binding to targets 1 and 2, fused to Fc stabilized by heterodimerization. CL and CH1 domains and VH and VL domains are switched, e.g., CH1 is fused in-line with VL, while CL is fused in-line with VH.

In some embodiments, the multi-specific binding protein is in a Fit-Ig form, which is a homodimeric constructs where Fab binding to antigen 2 is fused to the N terminus of HC of Fab that binds to antigen 1. The construct contains wild-type Fc.

Additional formats of the multi-specific binding proteins can be devised by combining various formats of CD33 binding-fragments described herein.

In certain embodiments of the present disclosure, the third component for the multi-specific binding proteins is an antibody constant region. In certain embodiments, each of the two immunoglobulin heavy chains of the antibody constant region includes a constant region with an amino acid sequence at least 90% (e.g., 90%, 91%, 92%, 93%, 94%, 95%, 96%, 97%, 98%, 99%, or 100%) identical to human IgG1 constant region. In certain embodiments, the amino acid sequence of one polypeptide chain of the antibody constant region differs from the amino acid sequence of an IgG1 constant region at one or more positions selected from the group consisting of Q347, Y349, L351, S354, E356, E357, K360, Q362, S364, T366, L368, K370, K392, T394, D399, S400, D401, F405, Y407, 1(409, T411 and K439; and the amino acid sequence of the other polypeptide chain of the antibody constant region differs from the amino acid sequence of an IgG1 constant region at one or more positions selected from the group consisting of Q347, Y349, L351, S354, E356, E357, S364, T366, L368, K370, N390, K392, T394, D399, D401, F405, Y407, K409, T411 and K439.

In certain embodiments of the present disclosure, the NKG2D-antigen binding site comprises:

(1) a heavy chain variable domain comprising an amino acid sequence at least 90% (e.g., 90%, 91%, 92%, 93%, 94%, 95%, 96%, 97%, 98%, 99%, or 100%) identical to SEQ ID NO:81 and a light chain variable domain comprising an amino acid sequence at least 90% (e.g., 90%, 91%, 92%, 93%, 94%, 95%, 96%, 97%, 98%, 99%, or 100%) identical to a SEQ ID NO:82 [ADI-29379];

(2) a heavy chain variable domain comprising an amino acid sequence at least 90% (e.g., 90%, 91%, 92%, 93%, 94%, 95%, 96%, 97%, 98%, 99%, or 100%) identical to SEQ ID NO:83 and a light chain variable domain comprising an amino acid sequence at least 90% (e.g., 90%, 91%, 92%, 93%, 94%, 95%, 96%, 97%, 98%, 99%, or 100%) identical to a SEQ ID NO:84 [ADI-29463];

(3) a heavy chain variable domain comprising an amino acid sequence at least 90% (e.g., 90%, 91%, 92%, 93%, 94%, 95%, 96%, 97%, 98%, 99%, or 100%) identical to SEQ ID NO:85 and a light chain variable domain comprising an amino acid sequence at least 90% (e.g., 90%, 91%, 92%, 93%, 94%, 95%, 96%, 97%, 98%, 99%, or 100%) identical to a SEQ ID NO:86 [ADI-27744];

(4) a heavy chain variable domain comprising an amino acid sequence at least 90% (e.g., 90%, 91%, 92%, 93%, 94%, 95%, 96%, 97%, 98%, 99%, or 100%) identical to SEQ ID NO:87 and a light chain variable domain comprising an amino acid sequence at least 90% (e.g., 90%, 91%, 92%, 93%, 94%, 95%, 96%, 97%, 98%, 99%, or 100%) identical to a SEQ ID NO:88 [ADI-27749];

(5) a heavy chain variable domain comprising an amino acid sequence at least 90% (e.g., 90%, 91%, 92%, 93%, 94%, 95%, 96%, 97%, 98%, 99%, or 100%) identical to SEQ ID NO:191 and a light chain variable domain comprising an amino acid sequence at least 90% (e.g., 90%, 91%, 92%, 93%, 94%, 95%, 96%, 97%, 98%, 99%, or 100%) identical to a SEQ ID NO:88 [A49MI]; or

(6) a heavy chain variable domain comprising an amino acid sequence at least 90% (e.g., 90%, 91%, 92%, 93%, 94%, 95%, 96%, 97%, 98%, 99%, or 100%) identical to SEQ ID NO:89 and a light chain variable domain comprising an amino acid sequence at least 90% (e.g., 90%, 91%, 92%, 93%, 94%, 95%, 96%, 97%, 98%, 99%, or 100%) identical to a SEQ ID NO:90 [ADI-29378].

In certain embodiments of the present disclosure, the NKG2D-antigen binding site comprises:

(1) a heavy chain variable domain comprising an amino acid sequence at least 90% (e.g., 90%, 91%, 92%, 93%, 94%, 95%, 96%, 97%, 98%, 99%, or 100%) identical to SEQ ID NO:124 and a light chain variable domain comprising an amino acid sequence at least 90% (e.g., 90%, 91%, 92%, 93%, 94%, 95%, 96%, 97%, 98%, 99%, or 100%) identical to a SEQ ID NO:125 [ADI-27705];

(2) a heavy chain variable domain comprising an amino acid sequence at least 90% (e.g., 90%, 91%, 92%, 93%, 94%, 95%, 96%, 97%, 98%, 99%, or 100%) identical to SEQ ID NO:129 and a light chain variable domain comprising an amino acid sequence at least 90% (e.g., 90%, 91%, 92%, 93%, 94%, 95%, 96%, 97%, 98%, 99%, or 100%) identical to a SEQ ID NO:130 [ADI-27724];

(3) a heavy chain variable domain comprising an amino acid sequence at least 90% (e.g., 90%, 91%, 92%, 93%, 94%, 95%, 96%, 97%, 98%, 99%, or 100%) identical to SEQ ID NO:131 and a light chain variable domain comprising an amino acid sequence at least 90% (e.g., 90%, 91%, 92%, 93%, 94%, 95%, 96%, 97%, 98%, 99%, or 100%) identical to a SEQ ID NO:132 [ADI-27740];

(4) a heavy chain variable domain comprising an amino acid sequence at least 90% (e.g., 90%, 91%, 92%, 93%, 94%, 95%, 96%, 97%, 98%, 99%, or 100%) identical to SEQ ID NO:133 and a light chain variable domain comprising an amino acid sequence at least 90% (e.g., 90%, 91%, 92%, 93%, 94%, 95%, 96%, 97%, 98%, 99%, or 100%) identical to a SEQ ID NO:134 [ADI-27741];

(5) a heavy chain variable domain comprising an amino acid sequence at least 90% (e.g., 90%, 91%, 92%, 93%, 94%, 95%, 96%, 97%, 98%, 99%, or 100%) identical to SEQ ID NO:135 and a light chain variable domain comprising an amino acid sequence at least 90% (e.g., 90%, 91%, 92%, 93%, 94%, 95%, 96%, 97%, 98%, 99%, or 100%) identical to a SEQ ID NO:136 [ADI-27743];

(6) a heavy chain variable domain comprising an amino acid sequence at least 90% (e.g., 90%, 91%, 92%, 93%, 94%, 95%, 96%, 97%, 98%, 99%, or 100%) identical to SEQ ID NO:137 and a light chain variable domain comprising an amino acid sequence at least 90% (e.g., 90%, 91%, 92%, 93%, 94%, 95%, 96%, 97%, 98%, 99%, or 100%) identical to a SEQ ID NO:138 [ADI-28153];

(7) a heavy chain variable domain comprising an amino acid sequence at least 90% (e.g., 90%, 91%, 92%, 93%, 94%, 95%, 96%, 97%, 98%, 99%, or 100%) identical to SEQ ID NO:139 and a light chain variable domain comprising an amino acid sequence at least 90% (e.g., 90%, 91%, 92%, 93%, 94%, 95%, 96%, 97%, 98%, 99%, or 100%) identical to a SEQ ID NO:140 [ADI-28226 (C26)];

(8) a heavy chain variable domain comprising an amino acid sequence at least 90% (e.g., 90%, 91%, 92%, 93%, 94%, 95%, 96%, 97%, 98%, 99%, or 100%) identical to SEQ ID NO:141 and a light chain variable domain comprising an amino acid sequence at least 90% (e.g., 90%, 91%, 92%, 93%, 94%, 95%, 96%, 97%, 98%, 99%, or 100%) identical to a SEQ ID NO:142;

(9) a heavy chain variable domain comprising an amino acid sequence at least 90% (e.g., 90%, 91%, 92%, 93%, 94%, 95%, 96%, 97%, 98%, 99%, or 100%) identical to SEQ ID NO:143 and a light chain variable domain comprising an amino acid sequence at least 90% (e.g., 90%, 91%, 92%, 93%, 94%, 95%, 96%, 97%, 98%, 99%, or 100%) identical to a SEQ ID NO:144;

(10) a heavy chain variable domain comprising an amino acid sequence at least 90% (e.g., 90%, 91%, 92%, 93%, 94%, 95%, 96%, 97%, 98%, 99%, or 100%) identical to SEQ ID NO:145 and a light chain variable domain comprising an amino acid sequence at least 90% (e.g., 90%, 91%, 92%, 93%, 94%, 95%, 96%, 97%, 98%, 99%, or 100%) identical to a SEQ ID NO:146;

(11) a heavy chain variable domain comprising an amino acid sequence at least 90% (e.g., 90%, 91%, 92%, 93%, 94%, 95%, 96%, 97%, 98%, 99%, or 100%) identical to SEQ ID NO:147 and a light chain variable domain comprising an amino acid sequence at least 90% (e.g., 90%, 91%, 92%, 93%, 94%, 95%, 96%, 97%, 98%, 99%, or 100%) identical to a SEQ ID NO:148;

(12) a heavy chain variable domain comprising an amino acid sequence at least 90% (e.g., 90%, 91%, 92%, 93%, 94%, 95%, 96%, 97%, 98%, 99%, or 100%) identical to SEQ ID NO:149 and a light chain variable domain comprising an amino acid sequence at least 90% (e.g., 90%, 91%, 92%, 93%, 94%, 95%, 96%, 97%, 98%, 99%, or 100%) identical to a SEQ ID NO:150;

(13) a heavy chain variable domain comprising an amino acid sequence at least 90% (e.g., 90%, 91%, 92%, 93%, 94%, 95%, 96%, 97%, 98%, 99%, or 100%) identical to SEQ ID NO:151 and a light chain variable domain comprising an amino acid sequence at least 90% (e.g., 90%, 91%, 92%, 93%, 94%, 95%, 96%, 97%, 98%, 99%, or 100%) identical to a SEQ ID NO:152;

(14) a heavy chain variable domain comprising an amino acid sequence at least 90% (e.g., 90%, 91%, 92%, 93%, 94%, 95%, 96%, 97%, 98%, 99%, or 100%) identical to SEQ ID NO:153 and a light chain variable domain comprising an amino acid sequence at least 90% (e.g., 90%, 91%, 92%, 93%, 94%, 95%, 96%, 97%, 98%, 99%, or 100%) identical to a SEQ ID NO:154;

(15) a heavy chain variable domain comprising an amino acid sequence at least 90% (e.g., 90%, 91%, 92%, 93%, 94%, 95%, 96%, 97%, 98%, 99%, or 100%) identical to SEQ ID NO:155 and a light chain variable domain comprising an amino acid sequence at least 90% (e.g., 90%, 91%, 92%, 93%, 94%, 95%, 96%, 97%, 98%, 99%, or 100%) identical to a SEQ ID NO:156;

(16) a heavy chain variable domain comprising an amino acid sequence at least 90% (e.g., 90%, 91%, 92%, 93%, 94%, 95%, 96%, 97%, 98%, 99%, or 100%) identical to SEQ ID NO:157 and a light chain variable domain comprising an amino acid sequence at least 90% (e.g., 90%, 91%, 92%, 93%, 94%, 95%, 96%, 97%, 98%, 99%, or 100%) identical to a SEQ ID NO:158;

(17) a heavy chain variable domain comprising an amino acid sequence at least 90% (e.g., 90%, 91%, 92%, 93%, 94%, 95%, 96%, 97%, 98%, 99%, or 100%) identical to SEQ ID NO:159 and a light chain variable domain comprising an amino acid sequence at least 90% (e.g., 90%, 91%, 92%, 93%, 94%, 95%, 96%, 97%, 98%, 99%, or 100%) identical to a SEQ ID NO:160;

(18) a heavy chain variable domain comprising an amino acid sequence at least 90% (e.g., 90%, 91%, 92%, 93%, 94%, 95%, 96%, 97%, 98%, 99%, or 100%) identical to SEQ ID NO:161 and a light chain variable domain comprising an amino acid sequence at least 90% (e.g., 90%, 91%, 92%, 93%, 94%, 95%, 96%, 97%, 98%, 99%, or 100%) identical to a SEQ ID NO:162;

(19) a heavy chain variable domain comprising an amino acid sequence at least 90% (e.g., 90%, 91%, 92%, 93%, 94%, 95%, 96%, 97%, 98%, 99%, or 100%) identical to SEQ ID NO:163 and a light chain variable domain comprising an amino acid sequence at least 90% (e.g., 90%, 91%, 92%, 93%, 94%, 95%, 96%, 97%, 98%, 99%, or 100%) identical to a SEQ ID NO:164;

(20) a heavy chain variable domain comprising an amino acid sequence at least 90% (e.g., 90%, 91%, 92%, 93%, 94%, 95%, 96%, 97%, 98%, 99%, or 100%) identical to SEQ ID NO:165 and a light chain variable domain comprising an amino acid sequence at least 90% (e.g., 90%, 91%, 92%, 93%, 94%, 95%, 96%, 97%, 98%, 99%, or 100%) identical to a SEQ ID NO:166;

(21) a heavy chain variable domain comprising an amino acid sequence at least 90% (e.g., 90%, 91%, 92%, 93%, 94%, 95%, 96%, 97%, 98%, 99%, or 100%) identical to SEQ ID NO:167 and a light chain variable domain comprising an amino acid sequence at least 90% (e.g., 90%, 91%, 92%, 93%, 94%, 95%, 96%, 97%, 98%, 99%, or 100%) identical to a SEQ ID NO:168;

(22) a heavy chain variable domain comprising an amino acid sequence at least 90% (e.g., 90%, 91%, 92%, 93%, 94%, 95%, 96%, 97%, 98%, 99%, or 100%) identical to SEQ ID NO:175 and a light chain variable domain comprising an amino acid sequence at least 90% (e.g., 90%, 91%, 92%, 93%, 94%, 95%, 96%, 97%, 98%, 99%, or 100%) identical to a SEQ ID NO:176;

(23) a heavy chain variable domain comprising an amino acid sequence at least 90% (e.g., 90%, 91%, 92%, 93%, 94%, 95%, 96%, 97%, 98%, 99%, or 100%) identical to SEQ ID NO:583 and a light chain variable domain comprising an amino acid sequence at least 90% (e.g., 90%, 91%, 92%, 93%, 94%, 95%, 96%, 97%, 98%, 99%, or 100%) identical to a SEQ ID NO:584; or

(24) a heavy chain variable domain comprising an amino acid sequence at least 90% (e.g., 90%, 91%, 92%, 93%, 94%, 95%, 96%, 97%, 98%, 99%, or 100%) identical to SEQ ID NO:585 and a light chain variable domain comprising an amino acid sequence at least 90% (e.g., 90%, 91%, 92%, 93%, 94%, 95%, 96%, 97%, 98%, 99%, or 100%) identical to a SEQ ID NO:580.

Table 2 lists peptide sequences of heavy chain variable domains and light chain variable domains that, in combination, can bind to NKG2D. Unless indicated otherwise, the CDR sequences provided in Table 2 are determined under Kabat. The NKG2D-binding domains can vary in their binding affinity to NKG2D, nevertheless, they all activate human NKG2D and NK cells.

TABLE 2

| | Heavy chain variable region amino acid | Light chain variable region |
|---|---|---|
| Clones | sequence | amino acid sequence |
| ADI- | QVQLVQSGAEVKKPGASVKVSCKAS | EIVMTQSPATLSVSPGERATLS |
| 29379 | GYTFTSYYMHWVRQAPGQGLEWMGI | CRASQSVSSNLAWYQQKPGQA |
| (E79) | INPSGGSTSYAQKFQGRVTMTRDTSTS | PRLLIYGASTRATGIPARFSGSG |
| | TVYMELSSLRSEDTAVYYCARGAPNY | SGTEFTLTISSLQSEDFAVYYCQ |
| | GDTTHDYYYMDVWGKGTTVTVSS | QYDDWPFTFGGGTKVEIK |
| | [SEQ ID NO: 81] | [SEQ ID NO: 82] |
| | CDR1: YTFTSYYMH [SEQ ID NO: 93] | CDR1: RASQSVSSNLA |
| | (non-Kabat) or SYYMH [SEQ ID NO: 566] | [SEQ ID NO: 96] |
| | CDR2: IINPSGGSTSYAQKFQG | CDR2: GASTRAT |
| | [SEQ ID NO: 94] | [SEQ ID NO: 97] |
| | CDR3: ARGAPNYGDTTHDYYYMDV | CDR3: QQYDDWPFT |
| | [SEQ ID NO: 95](non-Kabat) or | [SEQ ID NO: 98] |
| | GAPNYGDTTHDYYYMDV [SEQ ID | |
| | NO: 567] | |
| ADI- | QVQLVQSGAEVKKPGASVKVSCKAS | EIVLTQSPGTLSLSPGERATLSC |
| 29463 | GYTFTGYYMHWVRQAPGQGLEWMG | RASQSVSSNLAWYQQKPGQAP |
| (F63) | WINPNSGGTNYAQKFQGRVTMTRDT | RLLIYGASTRATGIPARFSGSGS |
| | SISTAYMELSRLRSDDTAVYYCARDT | GTEFTLTISSLQSEDFAVYYCQ |
| | GEYYDTDDHGMDVWGQGTTVTVSS | QDDYWPPTFGGGTKVEIK |
| | [SEQ ID NO: 83] | [SEQ ID NO: 84] |
| | CDR1: YTFTGYYMH [SEQ ID NO: 99] | CDR1: RASQSVSSNLA |
| | (non-Kabat) or GYYMH [SEQ ID | [SEQ ID NO: 102] |
| | NO: 568] | CDR2: GASTRAT |
| | CDR2: WINPNSGGTNYAQKFQG | [SEQ ID NO: 103] |
| | [SEQ ID NO: 100] | CDR3: QQDDYWPPT |
| | CDR3: ARDTGEYYDTDDHGMDV | [SEQ ID NO: 104] |
| | [SEQ ID NO: 101](non-Kabat) or | |
| | DTGEYYDTDDHGMDV [SEQ ID | |
| | NO: 569] | |
| ADI- | EVQLLESGGGLVQPGGSLRLSCAASG | DIQMTQSPSSVSASVGDRVTIT |
| 27744 | FTFSSYAMSWVRQAPGKGLEWVSAIS | CRASQGIDSWLAWYQQKPGK |
| (A44) | GSGGSTYYADSVKGRFTISRDNSKNT | APKLLIYAASSLQSGVPSRFSGS |
| | LYLQMNSLRAEDTAVYYCAKDGGYY | GSGTDFTLTISSLQPEDFATYYC |
| | DSGAGDYWGQGTLVTVSS | QQGVSYPRTFGGGTKVEIK |
| | [SEQ ID NO: 85] | [SEQ ID NO: 86] |
| | CDR1: FTFSSYAMS [SEQ ID NO: 105] | CDR1: RASQGIDSWLA |
| | (non-Kabat) or SYAMS [SEQ ID NO: 570] | [SEQ ID NO: 108] |
| | CDR2: AISGSGGSTYYADSVKG | CDR2: AASSLQS |
| | [SEQ ID NO: 106] | [SEQ ID NO: 109] |
| | CDR3: AKDGGYYDSGAGDY | CDR3: QQGVSYPRT |
| | [SEQ ID NO: 107](non-Kabat) or | [SEQ ID NO: 110] |
| | DGGYYDSGAGDY | |
| | [SEQ ID NO: 571] | |
| ADI- | EVQLVESGGGLVKPGGSLRLSCAASG | DIQMTQSPSSVSASVGDRVTIT |
| 27749 | FTFSSYSMNWVRQAPGKGLEWVSSIS | CRASQGISSWLAWYQQKPGKA |
| (A49) | SSSSYIYYADSVKGRFTISRDNAKNSL | PKLLIYAASSLQSGVPSRFSGSG |
| | YLQMNSLRAEDTAVYYCARGAPMGA | SGTDFTLTISSLQPEDFATYYCQ |
| | AAGWFDPWGQGTLVTVSS | QGVSFPRTFGGGTKVEIK |
| | [SEQ ID NO: 87] | [SEQ ID NO: 88] |
| | CDR1: FTFSSYSMN [SEQ ID NO: 111]or | CDR1: RASQGISSWLA |
| | SYSMN [SEQ ID NO: 192] | [SEQ ID NO: 114] |
| | CDR2: SISSSSSYIYYADSVKG | CDR2: AASSLQS |
| | [SEQ ID NO: 112] | [SEQ ID NO: 115] |
| | CDR3: ARGAPMGAAAGWFDP | CDR3: QQGVSFPRT |
| | [SEQ ID NO: 113]or | [SEQ ID NO: 116] |
| | GAPMGAAAGWFDP [SEQ ID NO: 193] | |
| A49MI | EVQLVESGGGLVKPGGSLRLSCAASG | DIQMTQSPSSVSASVGDRVTIT |
| | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPIGA | SGTDFTLTISSLQPEDFATYYCQ |
| | AAGWFDPWGQGTLVTVSS [SEQ ID | QGVSFPRTFGGGTKVEIK |
| | NO: 191] | [SEQ ID NO: 88] |
| | CDR1: FTFSSYSMN [SEQ ID NO: 111]or | CDR1: RASQGISSWLA |
| | SYSMN [SEQ ID NO: 192] | [SEQ ID NO: 114] |
| | CDR2: SISSSSSYIYYADSVKG | CDR2: AASSLQS |
| | [SEQ ID NO: 112] | [SEQ ID NO: 115] |
| | CDR3: ARGAPIGAAAGWFDP [SEQ ID | CDR3: QQGVSFPRT |
| | NO: 194] or GAPIGAAAGWFDP | [SEQ ID NO: 116] |
| | [SEQ ID NO: 195] | |
| A49MQ | EVQLVESGGGLVKPGGSLRLSCAASG | DIQMTQSPSSVSASVGDRVTIT |
| | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPQGA | SGTDFTLTISSLQPEDFATYYCQ |
| | AAGWFDPWGQGTLVTVSS | QGVSFPRTFGGGTKVEIK |
| | (SEQ ID NO: 586) | (SEQ ID NO: 88) |
| | CDR1 (non-Kabat)(SEQ ID NO: 111) - | CDR1 (SEQ ID NO: 114) - |
| | FTFSSYSMN or CDR1 (SEQ ID NO: 192) | RASQGISSWLA |
| | - SYSMN | CDR2 (SEQ ID NO: 115) - |
| | CDR2 (SEQ ID NO: 112) - | AASSLQS |
| | SISSSSSYIYYADSVKG | CDR3 (SEQ ID NO: 116) - |
| | CDR3 (non-Kabat)(SEQ ID NO: 587) - | QQGVSFPRT |
| | ARGAPQGAAAGWFDP or CDR3 (SEQ | |
| | ID NO: 588) - GAPQGAAAGWFDP | |
| A49ML | EVQLVESGGGLVKPGGSLRLSCAASG | DIQMTQSPSSVSASVGDRVTIT |
| | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPLGA | SGTDFTLTISSLQPEDFATYYCQ |
| | AAGWFDPWGQGTLVTVSS | QGVSFPRTFGGGTKVEIK |
| | (SEQ ID NO: 589) | (SEQ ID NO: 88) |
| | CDR1 (non-Kabat)(SEQ ID NO: 111) - | CDR1 (SEQ ID NO: 114) - |
| | FTFSSYSMN or CDR1 (SEQ ID NO: 192) | RASQGISSWLA |
| | - SYSMN | CDR2 (SEQ ID NO: 115) - |
| | CDR2 (SEQ ID NO: 112) - | AASSLQS |
| | SISSSSSYIYYADSVKG | CDR3 (SEQ ID NO: 116) - |
| | CDR3 (non-Kabat)(SEQ ID NO: 590) - | QQGVSFPRT |
| | ARGAPLGAAAGWFDP or CDR3 (SEQ | |
| | ID NO: 591) - GAPLGAAAGWFDP | |
| A49MF | EVQLVESGGGLVKPGGSLRLSCAASG | DIQMTQSPSSVSASVGDRVTIT |
| | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPFGA | SGTDFTLTISSLQPEDFATYYCQ |
| | AAGWFDPWGQGTLVTVSS | QGVSFPRTFGGGTKVEIK |
| | (SEQ ID NO: 592) | [SEQ ID NO: 88] |
| | CDR1 (non-Kabat)(SEQ ID NO: 111) - | CDR1: RASQGISSWLA |
| | FTFSSYSMN or CDR1 (SEQ ID NO: 192) | [SEQ ID NO: 114] |
| | - SYSMN | CDR2: AASSLQS |
| | CDR2 (SEQ ID NO: 112) - | [SEQ ID NO: 115] |
| | SISSSSSYIYYADSVKG | CDR3: QQGVSFPRT |
| | CDR3 (non-Kabat)(SEQ ID NO: 593) - | [SEQ ID NO: 116] |
| | ARGAPFGAAAGWFDP or CDR3 (SEQ | |
| | ID NO: 594) - GAPFGAAAGWFDP | |
| A49MV | EVQLVESGGGLVKPGGSLRLSCAASG | DIQMTQSPSSVSASVGDRVTIT |
| | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPVGA | SGTDFTLTISSLQPEDFATYYCQ |
| | AAGWFDPWGQGTLVTVSS | QGVSFPRTFGGGTKVEIK |
| | (SEQ ID NO: 595) | [SEQ ID NO: 88] |
| | CDR1 (non-Kabat)(SEQ ID NO: 111) - | CDR1: RASQGISSWLA |
| | FTFSSYSMN or CDR1 (SEQ ID NO: 192) | [SEQ ID NO: 114] |
| | - SYSMN | CDR2: AASSLQS |
| | CDR2 (SEQ ID NO: 112) - | [SEQ ID NO: 115] |
| | SISSSSSYIYYADSVKG | CDR3: QQGVSFPRT |
| | CDR3 (non-Kabat)(SEQ ID NO: 596) - | [SEQ ID NO: 116] |
| | ARGAPVGAAAGWFDP or CDR3 (SEQ | |
| | ID NO: 597) - GAPVGAAAGWFDP | |
| A49- | EVQLVESGGGLVKPGGSLRLSCAASG | DIQMTQSPSSVSASVGDRVTIT |
| consensus | FTFSSYSMNWVRQAPGKGLEWVSSIS | CRASQGISSWLAWYQQKPGKA |
| | SSSSYIYYADSVKGRFTISRDNAKNSL | PKLLIYAASSLQSGVPSRFSGSG |
| | YLQMNSLRAEDTAVYYCARGAPXGA | SGTDFTLTISSLQPEDFATYYCQ |
| | AAGWFDPWGQGTLVTVSS, wherein X | QGVSFPRTFGGGTKVEIK |
| | is M, L, I, V, Q, or F | [SEQ ID NO: 88] |
| | (SEQ ID NO: 91) | CDR1: RASQGISSWLA |
| | CDR1 (non-Kabat)(SEQ ID NO: 111) - | [SEQ ID NO: 114] |
| | FTFSSYSMN or CDR1 (SEQ ID NO: 192) | CDR2: AASSLQS |
| | - SYSMN | [SEQ ID NO: 115] |
| | CDR2 (SEQ ID NO: 112) - | CDR3: QQGVSFPRT |
| | SISSSSSYIYYADSVKG | [SEQ ID NO: 116] |
| | CDR3 (non-Kabat)(SEQ ID NO: 92) - | |
| | ARGAPXGAAAGWFDP or CDR3 (SEQ | |
| | ID NO: 123) - GAPXGAAAGWFDP, | |
| | wherein X is M, L, I, V, Q, or F | |
| ADI- | QVQLVQSGAEVKKPGASVKVSCKAS | EIVLTQSPATLSLSPGERATLSC |
| 29378 | GYTFTSYYMHWVRQAPGQGLEWMGI | RASQSVSSYLAWYQQKPGQAP |
| (E78) | INPSGGSTSYAQKFQGRVTMTRDTSTS | RLLIYDASNRATGIPARFSGSGS |
| | TVYMELSSLRSEDTAVYYCAREGAGF | GTDFTLTISSLEPEDFAVYYCQ |
| | AYGMDYYYMDVWGKGTTVTVSS | QSDNWPFTFGGGTKVEIK |
| | [SEQ ID NO: 89] | [SEQ ID NO: 90] |
| | CDR1: YTFTSYYMH [SEQ ID NO: 117] | CDR1: RASQSVSSYLA |
| | (non-Kabat) or SYYMH [SEQ ID NO: 572] | [SEQ ID NO: 120] |
| | CDR2: IINPSGGSTSYAQKFQG | CDR2: DASNRAT |
| | [SEQ ID NO: 118] | [SEQ ID NO: 121] |
| | CDR3: AREGAGFAYGMDYYYMDV | CDR3: QQSDNWPFT |
| | [SEQ ID NO: 119](non-Kabat) or | [SEQ ID NO: 122] |
| | EGAGFAYGMDYYYMDV [SEQ ID | |
| | NO: 573] | |
| ADI- | QVQLQQWGAGLLKPSETLSLTCAVY | DIQMTQSPSTLSASVGDRVTIT |
| 27705 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYNSYPITFGGGTKVEIK |
| | [SEQ ID NO: 124] | [SEQ ID NO: 125] |
| | CDR1: GSFSGYYWS [SEQ ID NO: 126] | |
| | (non-Kabat) or GYYWS [SEQ ID NO: 574] | |
| | CDR2: EIDHSGSTNYNPSLKS | |
| | [SEQ ID NO: 127] | |
| | CDR3: ARARGPWSFDP [SEQ ID | |
| | NO: 128](non-Kabat) or ARGPWSFDP | |
| | [SEQ ID NO: 575] | |
| ADI- | QVQLQQWGAGLLKPSETLSLTCAVY | EIVLTQSPGTLSLSPGERATLSC |
| 27724 | GGSFSGYYWSWIRQPPGKGLEWIGEI | RASQSVSSSYLAWYQQKPGQA |
| | DHSGSTNYNPSLKSRVTISVDTSKNQF | PRLLIYGASSRATGIPDRFSGSG |
| | SLKLSSVTAADTAVYYCARARGPWSF | SGTDFTLTISRLEPEDFAVYYC |
| | DPWGQGTLVTVSS | QQYGSSPITFGGGTKVEIK |
| | [SEQ ID NO: 129] | [SEQ ID NO: 130] |
| ADI- | QVQLQQWGAGLLKPSETLSLTCAVY | DIQMTQSPSTLSASVGDRVTIT |
| 27740 | GGSFSGYYWSWIRQPPGKGLEWIGEI | CRASQSIGSWLAWYQQKPGKA |
| (A40) | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYHSFYTFGGGTKVEIK |
| | [SEQ ID NO: 131] | [SEQ ID NO: 132] |
| ADI- | QVQLQQWGAGLLKPSETLSLTCAVY | DIQMTQSPSTLSASVGDRVTIT |
| 27741 | GGSFSGYYWSWIRQPPGKGLEWIGEI | CRASQSIGSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QSNSYYTFGGGTKVEIK |
| | [SEQ ID NO: 133] | [SEQ ID NO: 134] |
| ADI- | QVQLQQWGAGLLKPSETLSLTCAVY | DIQMTQSPSTLSASVGDRVTIT |
| 27743 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYNSYPTFGGGTKVEIK |
| | [SEQ ID NO: 135] | [SEQ ID NO: 136] |
| ADI- | QVQLQQWGAGLLKPSETLSLTCAVY | ELQMTQSPSSLSASVGDRVTIT |
| 28153 | GGSFSGYYWSWIRQPPGKGLEWIGEI | CRTSQSISSYLNWYQQKPGQPP |
| | DHSGSTNYNPSLKSRVTISVDTSKNQF | KLLIYWASTRESGVPDRFSGSG |
| | SLKLSSVTAADTAVYYCARARGPWG | SGTDFTLTISSLQPEDSATYYCQ |
| | FDPWGQGTLVTVS | SQSYDIPYTFGQGTKLEIK |
| | [SEQ ID NO: 137] | [SEQ ID NO: 138] |
| ADI- | QVQLQQWGAGLLKPSETLSLTCAVY | DIQMTQSPSTLSASVGDRVTIT |
| 28226 | GGSFSGYYWSWIRQPPGKGLEWIGEI | CRASQSISSWLAWYQQKPGKA |
| (C26) | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYGSFPITFGGGTKVEIK |
| | [SEQ ID NO: 139] | [SEQ ID NO: 140] |
| ADI- | QVQLQQWGAGLLKPSETLSLTCAVY | DIQMTQSPSTLSASVGDRVTIT |
| 28154 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTDFTLTISSLQPDDFATYYC |
| | DPWGQGTLVTVSS | QQSKEVPWTFGQGTKVEIK |
| | [SEQ ID NO: 141] | [SEQ ID NO: 142] |
| ADI- | QVQLQQWGAGLLKPSETLSLTCAVY | DIQMTQSPSTLSASVGDRVTIT |
| 29399 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYNSFPTFGGGTKVEIK |
| | [SEQ ID NO: 143] | [SEQ ID NO: 144] |
| ADI- | QVQLQQWGAGLLKPSETLSLTCAVY | DIQMTQSPSTLSASVGDRVTIT |
| 29401 | GGSFSGYYWSWIRQPPGKGLEWIGEI | CRASQSIGSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYDIYPTFGGGTKVEIK |
| | [SEQ ID NO: 145] | [SEQ ID NO: 146] |
| ADI- | QVQLQQWGAGLLKPSETLSLTCAVY | DIQMTQSPSTLSASVGDRVTIT |
| 29403 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYDSYPTFGGGTKVEIK |
| | [SEQ ID NO: 147] | [SEQ ID NO: 148] |
| ADI- | QVQLQQWGAGLLKPSETLSLTCAVY | DIQMTQSPSTLSASVGDRVTIT |
| 29405 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYGSFPTFGGGTKVEIK |
| | [SEQ ID NO: 149] | [SEQ ID NO: 150] |
| ADI- | QVQLQQWGAGLLKPSETLSLTCAVY | DIQMTQSPSTLSASVGDRVTIT |
| 29407 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW | SFSGTEFTLTISSLQPDDFATYYCQ |
| | DPWGQGTLVTVSS | QYQSFPTFGGGTKVEIK |
| | [SEQ ID NO: 151] | [SEQ ID NO: 152] |
| ADI- | QVQLQQWGAGLLKPSETLSLTCAVY | DIQMTQSPSTLSASVGDRVTIT |
| 29419 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYSSFSTFGGGTKVEIK |
| | [SEQ ID NO: 153] | [SEQ ID NO: 154] |
| ADI- | QVQLQQWGAGLLKPSETLSLTCAVY | DIQMTQSPSTLSASVGDRVTIT |
| 29421 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYESYSTFGGGTKVEIK |
| | [SEQ ID NO: 155] | [SEQ ID NO: 156] |
| ADI- | QVQLQQWGAGLLKPSETLSLTCAVY | DIQMTQSPSTLSASVGDRVTIT |
| 29424 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYDSFITFGGGTKVEIK |
| | [SEQ ID NO: 157] | [SEQ ID NO: 158] |
| ADI- | QVQLQQWGAGLLKPSETLSLTCAVY | DIQMTQSPSTLSASVGDRVTIT |
| 29425 | GGSFSGYYWSWIRQPPGKGLEWIGEI | CRASQSISSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYQSYPTFGGGTKVEIK |
| | [SEQ ID NO: 159] | [SEQ ID NO: 160] |
| ADI- | QVQLQQWGAGLLKPSETLSLTCAVY | DIQMTQSPSTLSASVGDRVTIT |
| 29426 | GGSFSGYYWSWIRQPPGKGLEWIGEI | CRASQSIGSWLAWYQQKPGKA |
| | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCQ |
| | DPWGQGTLVTVSS | QYHSFPTFGGGTKVEIK |
| | [SEQ ID NO: 161] | [SEQ ID NO: 162] |
| ADI- | QVQLQQWGAGLLKPSETLSLTCAVY | DIQMTQSPSTLSASVGDRVTIT |
| 29429 | GGSFSGYYWSWIRQPPGKGLEWIGEI | CRASQSIGSWLAWYQQKPGKA |
| | DHSGSTNYNPSLKSRVTISVDTSKNQF | PKLLIYKASSLESGVPSRFSGSG |
| | SLKLSSVTAADTAVYYCARARGPW SF | SGTEFTLTISSLQPDDFATYYCQ |
| | DPWGQGTLVTVSS | QYELYSYTFGGGTKVEIK |
| | [SEQ ID NO: 163] | [SEQ ID NO: 164] |
| ADI- | QVQLQQWGAGLLKPSETLSLTCAVY | DIQMTQSPSTLSASVGDRVTIT |
| 29447 | GGSFSGYYWSWIRQPPGKGLEWIGEI | CRASQSISSWLAWYQQKPGKA |
| (F47) | DHSGSTNYNPSLKSRVTISVDTSKNQF | PKLLIYKASSLESGVPSRFSGSG |
| | SLKLSSVTAADTAVYYCARARGPW SF | SGTEFTLTISSLQPDDFATYYCQ |
| | DPWGQGTLVTVSS | QYDTFITFGGGTKVEIK |
| | [SEQ ID NO: 165] | [SEQ ID NO: 166] |
| ADI- | QVQLVQSGAEVKKPGSSVKVSCKAS | DIVMTQSPDSLAVSLGERATIN |
| 27727 | GGTFSSYAISWVRQAPGQGLEWMGGI | CKSSQSVLYSSNNKNYLAWYQ |
| | IPIFGTANYAQKFQGRVTITADESTST | QKPGQPPKLLIYWASTRESGVP |
| | AYMELSSLRSEDTAVYYCARGDSSIR | DRFSGSGSGTDFTLTISSLQAED |
| | HAYYYYGMDVWGQGT TVTVSS | VAVYYCQQYYSTPITFGGGTK |
| | [SEQ ID NO: 167] | VEIK |
| | CDR1: GTFSSYAIS [SEQ ID NO: 169] | [SEQ ID NO: 168] |
| | (non-Kabat) or SYAIS [SEQ ID NO: 576] | CDR1: KSSQSVLYSSNNKNYLA |
| | CDR2: GIIPIFGTANYAQKFQG [SEQ ID | [SEQ ID NO: 172] |
| | NO: 170] | CDR2: WASTRES [SEQ ID |
| | CDR3: ARGDSSIRHAYYYYGMDV | NO: 173] |
| | [SEQ ID NO: 171](non-Kabat) or | CDR3: QQYYSTPIT [SEQ ID |
| | GDSSIRHAYYYYGMDV [SEQ ID | NO: 174] |
| | NO: 577] | |
| ADI- | QLQLQESGPGLVKPSETLSLTCTVSGG | EIVLTQSPATLSLSPGERATLSC |
| 29443 | SISSSSYYWGWIRQPPGKGLEWIGSIY | RASQSVSRYLAWYQQKPGQAP |
| (F43) | YSGSTYYNPSLKSRVTISVDTSKNQFS | RLLIYDASNRATGIPARFSGSGS |
| | LKLSSVTAADTAVYYCARGSDRFHPY | GTDFTLTISSLEPEDFAVYYCQ |
| | FDYWGQGTLVTVSS | QFDTWPPTFGGGTKVEIK |
| | [SEQ ID NO: 175] | [SEQ ID NO: 176] |
| | CDR1: GSISSSSYYWG | CDR1: RASQSVSRYLA |
| | [SEQ ID NO: 177](non-Kabat) or | [SEQ ID NO: 180] |
| | SSSYYWG [SEQ ID NO: 578] | CDR2: DASNRAT |
| | CDR2: SIYYSGSTYYNPSLKS | [SEQ ID NO: 581] |
| | [SEQ ID NO: 178] | CDR3: QQFDTWPPT |
| | CDR3: ARGSDRFHPYFDY | [SEQ ID NO: 582] |
| | [SEQ ID NO: 179](non-Kabat) or | |
| | GSDRFHPYFDY [SEQ ID NO: 579] | |
| ADI- | QVQLQQWGAGLLKPSETLSLTCAVY | DIQMTQSPSTLSASVGDRVTIT |
| 29404 | GGSFSGYYWSWIRQPPGKGLEWIGEI | CRASQSISSWLAWYQQKPGKA |
| (F04) | DHSGSTNYNPSLKSRVTISVDTSKNQF | PKLLIYKASSLESGVPSRFSGSG |
| | SLKLSSVTAADTAVYYCARARGPWSF | SGTEFTLTISSLQPDDFATYYCE |
| | DPWGQGTLVTVSS | QYDSYPTFGGGTKVEIK |
| | [SEQ ID NO: 583] | [SEQ ID NO: 584] |
| ADI- | QVQLVQSGAEVKKPGSSVKVSCKAS | DIVMTQSPDSLAVSLGERATIN |
| 28200 | GGTFSSYAISWVRQAPGQGLEWMGGI | CESSQSLLNSGNQKNYLTWYQ |
| | IPIFGTANYAQKFQGRVTITADESTST | QKPGQPPKPLIYWASTRESGVP |
| | AYMELSSLRSEDTAVYYCARRGRKAS | DRFSGSGSGTDFTLTISSLQAED |
| | GSFYYYYGMDVWGQGTTVTVSS | VAVYYCQNDYSYPYTFGQGTK |
| | [SEQ ID NO: 585] | LEIK |
| | | [SEQ ID NO: 580] |

The antibody molecule may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In some embodiments, the antibody constant domain comprises a CH2 domain and a CH3 domain of an IgG antibody, for example, a human IgG1 antibody. In some embodiments, mutations are introduced in the antibody constant domain to enable heterdimerization with another antibody constant domain. For example, if the antibody constant domain is derived from the constant domain of a human IgG1, the antibody constant domain can comprise an amino acid sequence at least 90% (e.g., 90%, 91%, 92%, 93%, 94%, 95%, 96%, 97%, 98%, 99%, or 100%) identical to amino acids 234-332 of a human IgG1 antibody, and differs at one or more positions selected from the group consisting of Q347, Y349, L351, 5354, E356, E357, K360, Q362, 5364, T366, L368, K370, N390, K392, T394, D399, 5400, D401, F405, Y407, K409, T411, and K439. All the amino acid positions in an Fc domain or hinge region disclosed herein are numbered according to EU numbering.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% (e.g., 90%, 91%, 92%, 93%, 94%, 95%, 96%, 97%, 98%, 99%, or 100%) identical to an antibody constant region, such as an IgG constant region including hinge, CH2 and CH3 domains with or without CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, an IgG2 constant region, IgG3 constant region, or IgG4 constant region. In some other embodiments, the amino acid sequence of the constant region is at least 90% (e.g., 90%, 91%, 92%, 93%, 94%, 95%, 96%, 97%, 98%, 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 3.

TABLE 3

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4

| | First Polypeptide | Second Polypeptide |
|---|---|---|
| Set 1 | K409W | D399V/F405T |
| Set 2 | Y3495 | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 5.

TABLE 5

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6

| First Polypeptide | Second Polypeptide |
|---|---|
| L351Y, D399R, D399K, | T366V, T366I, T366L, T366M, N390D, |
| S400K, S400R, | N390E, K392L, K392M, K392V, K392F |
| Y407A, Y407I, Y407V | K392D, K392E, K409F, K409W, T411D |
| | and T411E |

Alternatively, at least one amino acid substitutions could be selected from the following set of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7

| First Polypeptide | Second Polypeptide |
|---|---|
| K392, K370, K409, or K439 | D399, E356, or E357 |

Alternatively, at least one amino acid substitutions could be selected from the following set of in Table 8, where the position(s) indicated in the First Polypeptide column is replaced by any known positively-charged amino acid, and the position(s) indicated in the Second Polypeptide Column is replaced by any known negatively-charged amino acid.

TABLE 8

| First Polypeptide | Second Polypeptide |
|---|---|
| D399, E356, or E357 | K409, K439, K370, or K392 |

Alternatively, amino acid substitutions could be selected from the following set of in Table 9.

TABLE 9

| First Polypeptide | Second Polypeptide |
|---|---|
| T350V, L351Y, F405A, and Y407V | T350V, T366L, K392L, and T394W |

Alternatively, or in addition, the structural stability of heterodimeric heavy chains within the multi-specific binding proteins can be increased by introducing S354C on either of the first or second polypeptide chain, and Y349C on the opposing polypeptide chain, which forms an artificial disulfide bridge within the interface of the two polypeptides.

In certain embodiments of the present disclosure, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In certain embodiments of the present disclosure, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In certain embodiments of the present disclosure, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, 5364, L368, K370, T394, D401, F405 and T411.

In certain embodiments of the present disclosure, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In certain embodiments of the present disclosure, the amino acid sequence of one polypeptide chain of the antibody constant region differs from the amino acid sequence of an IgG1 constant region at one or more positions selected from the group consisting of L351, D399, 5400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In certain embodiments of the present disclosure, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In certain embodiments of the present disclosure, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In certain embodiments of the present disclosure, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In certain embodiments of the present disclosure, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In certain embodiments of the present disclosure, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In certain embodiments of the present disclosure, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In certain embodiments of the present disclosure, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In certain embodiments of the present disclosure, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In certain embodiments of the present disclosure, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In certain embodiments of the present disclosure, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In certain embodiments of the present disclosure, the amino acid sequence of one polypeptide chain of the antibody constant region differs from the amino acid sequence of an IgG1 constant region by 0347R, D399V and F405T substitutions and wherein the amino acid sequence of the other polypeptide chain of the antibody constant region differs from the amino acid sequence of an IgG1 constant region by K360E and K409W substitutions.

In certain embodiments of the present disclosure, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In certain embodiments of the present disclosure, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In certain embodiments of the present disclosure, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In certain embodiments of the present disclosure, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

Listed below are examples of CD33-binding F3′-TriNKETs comprising: a CD33-binding single-chain variable fragment (scFv) linked to an Fc domain via a hinge comprising Ala-Ser; and an NKG2D-binding Fab fragment (“A49” or “A49MI”) comprising a heavy chain portion comprising an heavy chain variable domain (SEQ ID NO:87 or SEQ ID NO:191) and a CH1 domain, and a light chain portion comprising a light chain variable domain (SEQ ID NO:88) and a light chain constant domain, wherein the heavy chain variable domain is connected to the CH1 domain, and the CH1 domain is connected to an Fc domain. The CDR sequences are underlined.

In each of the examples, the Fc domain linked to the CD33-binding scFv comprises Q347R, D399V, and F405T substitutions for forming a heterodimer with the Fc domain linked to the Fab comprising K360E and K409W substitutions. These substitutions in the Fc domains are bold-underlined in the sequences described below. Alternatively, in an exemplary embodiment, the Fc domain linked to the NKG2D-binding Fab fragment includes the mutations of Q347R, D399V, and F405T, and the Fc domain linked to the CD33-binding scFv comprises matching mutations K360E and K409W for forming a heterodimer.

Additionally, the Fc domain linked to the CD33-binding scFv comprises S354C substitution, and the Fc domain linked to the Fab comprises Y349C substitution, thereby stabilizing the interaction between the two Fc domains via a S-S bridge. These substitutions in the Fc domains are bold-italics-underlined in the sequences below.

Alternatively, in an exemplary embodiment, the Fc domain linked to the NKG2D-binding Fab fragment includes a S354C substitution in the CH3 domain, which forms a disulfide bond with a Y349C substitution on the Fc linked to the CD33-binding scFv.

A CD33-binding scFv of the present disclosure can include a heavy chain variable domain connected to a light chain variable domain with a (G45)₄ linker. The scFv is linked to an Fc domain via a hinge comprising Ala-Ser (bolded-underlined). SEQ ID NOs:188, 198, and 206-223 are exemplary sequences of such CD33-binding scFv polypeptides. The V_{L} and V_{H} comprised within the scFv (e.g., SEQ ID NOs:188, 198 or 206-223) contain 100V_{L}-44V_{H} S-S bridge (resulting from G100C and G44C substitutions, respectively) (cysteine residues are in bold-italics-underlined in the sequences below). (G4S)₄ is the bold-underlined sequence GGGGSGGGGSGGGGSGGGGS [SEQ ID NO:186] in SEQ ID NOs:188, 198, and 206-243.

Exemplary sequences of CD33-binding scFvs linked to an Fc domain via a hinge comprising Ala-Ser are provided below.

Ab1 scFv (LC-HC)-Fc (Q347R, D399V, F405T, and S354C
substitutions)
[SEQ ID NO: 224]
DIQMTQSPSTLSASVGDRVTITCRASQSISSWLAWYQQKPGKAPKLLIYDASSLESGV
PSRFSGSGSGTEFTLTISSLQPDDFATYYCQQYESFPTFG [symbol] GTKVEIKGGGGSGGGGS
GGGGSGGGGSEVQLVESGGGLVQPGGSLRLSCAASGFTFSSYGMSWVRQAPGK [symbol] L
EWVANIKQDGSEKYYVDSVKGRFTISRDNAKNSLYLQMNSLRAEDTAVYYCAREG
GPYYDSSGYFVYYGMDVWGQGTTVTVSSASDKTHTCPPCPAPELLGGPSVFLFPPKP
KDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPRVYTLPP [symbol] RDELT
KNQVSLTCLVKGFYPSDIAVEWESNGQPENNYKTTPPVLVSDGSFTLYSKLTVDKSR
WQQGNVFSCSVMHEALHNHYTQKSLSLSPG
Ab1 scFv (HC-LC)-Fc (Q347R, D399V, F405T, and S354C
substitutions)
[SEQ ID NO: 225]
EVQLVESGGGLVQPGGSLRLSCAASGFTFSSYGMSWVRQAPGK [symbol] LEWVANIKQDGS
EKYYVDSVKGRFTISRDNAKNSLYLQMNSLRAEDTAVYYCAREGGPYYDSSGYFVY
YGMDVWGQGTTVTVSSGGGGSGGGGSGGGGSGGGGSDIQMTQSPSTLSASVGDR
VTITCRASQSISSWLAWYQQKPGKAPKLLIYDASSLESGVPSRFSGSGSGTEFTLTISS
LQPDDFATYYCQQYESFPTFG [symbol] GTKVEIKASDKTHTCPPCPAPELLGGPSVFLFPPKP
KDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPRVYTLPP [symbol] RDELT
KNQVSLTCLVKGFYPSDIAVEWESNGQPENNYKTTPPVLVSDGSFTLYSKLTVDKSR
WQQGNVFSCSVMHEALHNHYTQKSLSLSPG
Ab2 scFv (LC-HC)-Fc (Q347R, D399V, F405T, and S354C
substitutions)
[SEQ ID NO: 226]
DIQMTQSPSTLSASVGDRVTITCRASQSISSWLAWYQQKPGKAPKLLIYEASSLESGV
PSRFSGSGSGTEFTLTISSLQPDDFATYYCQQLESYPLTFG [symbol] GTKVEIKGGGGSGGGG
SGGGGSGGGGSEVQLVESGGGLVQPGGSLRLSCAASGFTFSSYWMSWVRQAPGK [symbol]
LEWVANIKQDGSEKYYVDSVKGRFTISRDNAKNSLYLQMNSLRAEDTAVYYCARPL
NAGELDVWGQGTMVTVSSASDKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQGNVFSCS
VMHEALHNHYTQKSLSLSPG
Ab2 scFv (HC-LC)-Fc (Q347R, D399V, F405T, and S354C
substitutions)
[SEQ ID NO: 227]
EVQLVESGGGLVQPGGSLRLSCAASGFTFSSYWMSWVRQAPGK [symbol] LEWVANIKQDG
SEKYYVDSVKGRFTISRDNAKNSLYLQMNSLRAEDTAVYYCARPLNAGELDVWGQ
GTMVTVSSGGGGSGGGGSGGGGSGGGGSDIQMTQSPSTLSASVGDRVTITCRASQ
SISSWLAWYQQKPGKAPKLLIYEASSLESGVPSRFSGSGSGTEFTLTISSLQPDDFATY
YCQQLESYPLTFG [symbol] GTKVEIKASDKTHTCPPCPAPELLGGPSVFLFPPKPKDTLMISRT
PEVTCVVVDVSHEDPEVKFNWYVDGVEVHNAKTKPREEQYNSTYRVVSVLTVLHQ
DWLNGKEYKCKVSNKALPAPIEKTISKAKGQPREPRVYTLPP [symbol] RDELTKNQVSLTCL
VKGFYPSDIAVEWESNGQPENNYKTTPPVLVSDGSFTLYSKLTVDKSRWQQGNVFS
CSVMHEALHNHYTQKSLSLSPG
H76 scFv (LC-HC)-Fc (Q347R, D399V, F405T, and S354C
substitutions)
[SEQ ID NO: 197]
DIQMTQSPSTLSASVGDRVTITCRASQSISSWLAWYQQKPGKAPKLLIYKASSLESGV
PSRFSGSGSGTEFTLTISSLQPDDFATYYCQQYDDLPTFG [symbol] GTKVEIKGGGGSGGGG
SGGGGSGGGGSEVQLLESGGGLVQPGGSLRLSCAASGFTFSKYTMSWVRQAPGK [symbol]
LEWVSAIVGSGESTYFADSVKGRFTISRDNSKNTLYLQMNSLRAEDTAVYYCAREG
GPYYDSSGYFVYYGMDVWGQGTTVTVSSASDKTHTCPPCPAPELLGGPSVFLFPPKP
KDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPRVYTLPP [symbol] RDELT
KNQVSLTCLVKGFYPSDIAVEWESNGQPENNYKTTPPVLVSDGSFTLYSKLTVDKSR
WQQGNVFSCSVMHEALHNHYTQKSLSLSPG
H76 scFv (HC-LC)-Fc (Q347R, D399V, F405T, and S354C
substitutions)
[SEQ ID NO: 228]
EVQLLESGGGLVQPGGSLRLSCAASGFTFSKYTMSWVRQAPGK [symbol] LEWVSAIVGSGE
STYFADSVKGRFTISRDNSKNTLYLQMNSLRAEDTAVYYCAREGGPYYDSSGYFVY
YGMDVWGQGTTVTVSSGGGGSGGGGSGGGGSGGGGSDIQMTQSPSTLSASVGDR
VTITCRASQSISSWLAWYQQKPGKAPKLLIYKASSLESGVPSRFSGSGSGTEFTLTISS
LQPDDFATYYCQQYDDLPTFG [symbol] GTKVEIKASDKTHTCPPCPAPELLGGPSVFLFPPKP
KDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPRVYTLPP [symbol] RDELT
KNQVSLTCLVKGFYPSDIAVEWESNGQPENNYKTTPPVLVSDGSFTLYSKLTVDK SR
WQQGNVFSCSVMHEALHNHYTQKSLSLSPG
H76 scFc (LC-HC)-Fc (K360E, K409W, and Y349C substitutions)
[SEQ ID NO: 243]
DIQMTQSPSTLSASVGDRVTITCRASQSISSWLAWYQQKPGKAPKLLIYKASSLESGV
PSRFSGSGSGTEFTLTISSLQPDDFATYYCQQYDDLPTFG [symbol] GTKVEIKGGGGSGGGG
SGGGGSGGGGSEVQLLESGGGLVQPGGSLRLSCAASGFTFSKYTMSWVRQAPGK [symbol]
LEWVSAIVGSGESTYFADSVKGRFTISRDNSKNTLYLQMNSLRAEDTAVYYCAREG
GPYYDSSGYFVYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQV [symbol] TLPPSRDELTENQVSLTCLVKGFY
PSDIAVEWESNGQPENNYKTTPPVLDSDGSFFLYSWLTVDKSRWQQGNVFSCSVMH
EALHNHYTQKSLSLSPG
Ab4 scFv (LC-HC)-Fc (Q347R, D399V, F405T, and S354C
substitutions)
[SEQ ID NO: 229]
DIVMTQSPLSLPVTPGEPASISCRSSQSLLYSNGYNYLDWYLQKPGQSPQLLIYLGSN
RASGVPDRFSGSGSGTDFTLKISRVEAEDVGVYYCMQDVALPITFG [symbol] GTKVEIKGGG
GSGGGGSGGGGSGGGGSQVQLVQSGAEVKKPGASVKVSCKASGYTFSDYYMHW
VRQAPGQ [symbol] LEWMGMINPSWGSTSYAQKFQGRVTMTRDTSTSTVYMELSSLRSEDT
AVYYCAREAADGFVGERYFDLWGRGTLVTVSSASDKTHTCPPCPAPELLGGPSVFLF
PPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPRVYTLPP [symbol] RD
ELTKNQVSLTCLVKGFYPSDIAVEWESNGQPENNYKTTPPVLVSDGSFTLYSKLTVD
KSRWQQGNVFSCSVMHEALHNHYTQKSLSLSPG
Ab4 scFv (HC-LC)-Fc (Q347R, D399V, F405T, and S354C
substitutions)
[SEQ ID NO: 230]
QVQLVQSGAEVKKPGASVKVSCKASGYTFSDYYMHWVRQAPGQ [symbol] LEWMGMINPS
WGSTSYAQKFQGRVTMTRDTSTSTVYMELSSLRSEDTAVYYCAREAADGFVGERYF
DLWGRGTLVTVSSGGGGSGGGGSGGGGSGGGGSDIVMTQSPLSLPVTPGEPASISC
RSSQSLLYSNGYNYLDWYLQKPGQSPQLLIYLGSNRASGVPDRFSGSGSGTDFTLKIS
RVEAEDVGVYYCMQDVALPITFG [symbol] GTKVEIKASDKTHTCPPCPAPELLGGPSVFLFPP
KPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPRVYTLPP [symbol] RDEL
TKNQVSLTCLVKGFYPSDIAVEWESNGQPENNYKTTPPVLVSDGSFTLYSKLTVDKS
RWQQGNVFSCSVMHEALHNHYTQKSLSLSPG
107 scFv (LC-HC)-Fc (Q347R, D399V, F405T, and S354C
substitutions)
[SEQ ID NO: 187]
DIQMTQSPSTLSASVGDRVTITCRASQSISSWLAWYQQKPGKAPKLLIYEASSLESGV
PSRFSGSGSGTEFTLTISSLQPDDFATYYCQQSQSYPPITFG [symbol] GTKVEIKGGGGSGGG
GSGGGGSGGGGSEVQLVESGGGLVQPGGSLRLSCAASGFTFGSYWMSWVRQAPG
K [symbol] LEWVATIKQDGSEKSYVDSVKGRFTISRDNAKNSLYLQMNSLRAEDTAVYYCAR
PLNAGELDVWGQGTMVTVSSASDKTHTCPPCPAPELLGGPSVFLFPPKPKDTLMISR
TPEVTCVVVDVSHEDPEVKFNWYVDGVEVHNAKTKPREEQYNSTYRVVSVLTVLH
QDWLNGKEYKCKVSNKALPAPIEKTISKAKGQPREPRVYTLPP [symbol] RDELTKNQVSLTC
LVKGFYPSDIAVEWESNGQPENNYKTTPPVLVSDGSFTLYSKLTVDKSRWQQGNVF
SCSVMHEALHNHYTQKSLSLSPG
107 scFv (HC-LC)-Fc (Q347R, D399V, F405T, and S354C
substitutions)
[SEQ ID NO: 231]
EVQLVESGGGLVQPGGSLRLSCAASGFTFGSYWMSWVRQAPGK [symbol] LEWVATIKQDG
SEKSYVDSVKGRFTISRDNAKNSLYLQMNSLRAEDTAVYYCARPLNAGELDVWGQ
GTMVTVSSGGGGSGGGGSGGGGSGGGGSDIQMTQSPSTLSASVGDRVTITCRASQ
SISSWLAWYQQKPGKAPKLLIYEASSLESGVPSRFSGSGSGTEFTLTISSLQPDDFATY
YCQQSQSYPPITFG [symbol] GTKVEIKASDKTHTCPPCPAPELLGGPSVFLFPPKPKDTLMISR
TPEVTCVVVDVSHEDPEVKFNWYVDGVEVHNAKTKPREEQYNSTYRVVSVLTVLH
QDWLNGKEYKCKVSNKALPAPIEKTISKAKGQPREPRVYTLPP [symbol] RDELTKNQVSLTC
LVKGFYPSDIAVEWESNGQPENNYKTTPPVLVSDGSFTLYSKLTVDKSRWQQGNVF
SCSVMHEALHNHYTQKSLSLSPG
107 scFc (LC-HC)-Fc (K360E, K409W, and Y349C substitutions)
[SEQ ID NO: 242]
DIQMTQSPSTLSASVGDRVTITCRASQSISSWLAWYQQKPGKAPKLLIYEASSLESGV
PSRFSGSGSGTEFTLTISSLQPDDFATYYCQQSQSYPPITFG [symbol] GTKVEIKGGGGSGGG
GSGGGGSGGGGSEVQLVESGGGLVQPGGSLRLSCAASGFTFGSYWMSWVRQAPG
K [symbol] LEWVATIKQDGSEKSYVDSVKGRFTISRDNAKNSLYLQMNSLRAEDTAVYYCAR
PLNAGELDVWGQGTMVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKALPAPIEKTISKAKGQPREPQV [symbol] TLPPSRDELTENQVSLTCLVKGFYPSDIAVEW
ESNGQPENNYKTTPPVLDSDGSFFLYSWLTVDKSRWQQGNVFSCSVMHEALHNHY
TQKSLSLSPG
Ab6 scFv (LC-HC)-Fc (Q347R, D399V, F405T, and S354C
substitutions)
[SEQ ID NO: 232]
DIQMTQSPSTLSASVGDRVTITCRASQSISSWLAWYQQKPGKAPKLLIYEASSLESGV
PSRFSGSGSGTEFTLTISSLQPDDFATYYCQQSQSYPPITFG [symbol] GTKVEIKGGGGSGGG
GSGGGGSGGGGSEVQLVESGGGLVQPGGSLRLSCAASGFTFPSYWMSWVRQAPGK [symbol]
LEWVATIKRDGSEKGYVDSVKGRFTISRDNAKNSLYLQMNSLRAEDTAVYYCARP
LNAGELDVWGQGTMVTVSSASDKTHTCPPCPAPELLGGPSVFLFPPKPKDTLMISRT
PEVTCVVVDVSHEDPEVKFNWYVDGVEVHNAKTKPREEQYNSTYRVVSVLTVLHQ
DWLNGKEYKCKVSNKALPAPIEKTISKAKGQPREPRVYTLPP [symbol] RDELTKNQVSLTCL
VKGFYPSDIAVEWESNGQPENNYKTTPPVLVSDGSFTLYSKLTVDKSRWQQGNVFS
CSVMHEALHNHYTQKSLSLSPG
Ab6 scFv (HC-LC)-Fc (Q347R, D399V, F405T, and S354C
substitutions)
[SEQ ID NO: 233]
EVQLVESGGGLVQPGGSLRLSCAASGFTFPSYWMSWVRQAPGK [symbol] LEWVATIKRDGS
EKGYVDSVKGRFTISRDNAKNSLYLQMNSLRAEDTAVYYCARPLNAGELDVWGQG
TMVTVSSGGGGSGGGGSGGGGSGGGGSDIQMTQSPSTLSASVGDRVTITCRASQSI
SSWLAWYQQKPGKAPKLLIYEASSLESGVPSRFSGSGSGTEFTLTISSLQPDDFATYY
CQQSQSYPPITFG [symbol] GTKVEIKASDKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQGNVFSCS
VMHEALHNHYTQKSLSLSPG
Ab7 scFv (LC-HC)-Fc (Q347R, D399V, F405T, and S354C
substitutions)
[SEQ ID NO: 234]
DIQMTQSPSSVSASVGDRVTITCRASQGIDSWLAWYQQKPGKAPKLLIYAASSLQSG
VPSRFSGSGSGTDFTLTISSLQPEDFATYYCQQAHSYPLTFG [symbol] GTKVEIKGGGGSGG
GGSGGGGSGGGGSQVQLVQSGAEVKKPGASVKVSCKASGYTFGTYYMHWVRQA
PGQ [symbol] LEWMGIINPSRGSTVYAQKFQGRVTMTRDTSTSTVYMELSSLRSEDTAVYYC
ARGAGYDDEDMDVWGKGTTVTVSSASDKTHTCPPCPAPELLGGPSVFLFPPKPKDT
LMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPRVYTLPP [symbol] RDELTKNQV
SLTCLVKGFYPSDIAVEWESNGQPENNYKTTPPVLVSDGSFTLYSKLTVDKSRWQQG
NVFSCSVMHEALHNHYTQKSLSLSPG
Ab7 scFv (HC-LC)-Fc (Q347R, D399V, F405T, and S354C
substitutions)
[SEQ ID NO: 235]
QVQLVQSGAEVKKPGASVKVSCKASGYTFGTYYMHWVRQAPGQ [symbol] LEWMGIINPSR
GSTVYAQKFQGRVTMTRDTSTSTVYMELSSLRSEDTAVYYCARGAGYDDEDMDV
WGKGTTVTVSSGGGGSGGGGSGGGGSGGGGSDIQMTQSPSSVSASVGDRVTITCR
ASQGIDSWLAWYQQKPGKAPKLLIYAASSLQSGVPSRFSGSGSGTDFTLTISSLQPED
FATYYCQQAHSYPLTFG [symbol] GTKVEIKASDKTHTCPPCPAPELLGGPSVFLFPPKPKDTL
MISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPRVYTLPP [symbol] RDELTKNQVS
LTCLVKGFYPSDIAVEWESNGQPENNYKTTPPVLVSDGSFTLYSKLTVDKSRWQQG
NVFSCSVMHEALHNHYTQKSLSLSPG
Ab8 scFv (LC-HC)-Fc (Q347R, D399V, F405T, and S354C
substitutions)
[SEQ ID NO: 236]
DIQMTQSPSTLSASVGDRVTITCRASNSISSWLAWYQQKPGKAPKLLIYEASSTKSGV
PSRFSGSGSGTEFTLTISSLQPDDFATYYCQQYDDLPTFG [symbol] GTKVEIKGGGGSGGGG
SGGGGSGGGGSEVQLVESGGGLVKPGGSLRLSCAASGFTFSSYAMSWVRQAPGK [symbol]
LEWVSSISSSSEGIYYADSVKGRFTISRDNAKNSLYLQMNSLRAEDTAVYYCAREGG
PYYDSSGYFVYYGMDVWGQGTTVTVSSASDKTHTCPPCPAPELLGGPSVFLFPPKPK
DTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPRVYTLPP [symbol] RDELTKN
QVSLTCLVKGFYPSDIAVEWESNGQPENNYKTTPPVLVSDGSFTLYSKLTVDKSRWQ
QGNVFSCSVMHEALHNHYTQKSLSLSPG
Ab8 scFv (HC-LC)-Fc (Q347R, D399V, F405T, and S354C
substitutions)
[SEQ ID NO: 237]
EVQLVESGGGLVKPGGSLRLSCAASGFTFSSYAMSWVRQAPGK [symbol] LEWVSSISSSSEGI
YYADSVKGRFTISRDNAKNSLYLQMNSLRAEDTAVYYCAREGGPYYDSSGYFVYY
GMDVWGQGTTVTVSSGGGGSGGGGSGGGGSGGGGSDIQMTQSPSTLSASVGDRV
TITCRASNSISSWLAWYQQKPGKAPKLLIYEASSTKSGVPSRFSGSGSGTEFTLTISSL
QPDDFATYYCQQYDDLPTFG [symbol] GTKVEIKASDKTHTCPPCPAPELLGGPSVFLFPPKPK
DTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPRVYTLPP [symbol] RDELTKN
QVSLTCLVKGFYPSDIAVEWESNGQPENNYKTTPPVLVSDGSFTLYSKLTVDKSRWQ
QGNVFSCSVMHEALHNHYTQKSLSLSPG
Ab9 scFv (LC-HC)-Fc (Q347R, D399V, F405T, and S354C
substitutions)
[SEQ ID NO: 238]
DIQMTQSPSSLSASVGDRVTITCRASQVIYSYLNWYQQKPGKAPKLLIYAASSLKSGV
PSRFSGSGSGTDFTLTISSLQPEDFATYYCQQVYDTPLTFG [symbol] GTKVEIKGGGGSGGG
GSGGGGSGGGGSEVQLVESGGGLVQPGGSLRLSCAASGFTFSSYWMSWVRQAPGK [symbol]
LEWVANINTDGSEVYYVDSVKGRFTISRDNAKNSLYLQMNSLRAEDTAVYYCARD
VGPGIAYQGHFDYWGQGTLVTVSSASDKTHTCPPCPAPELLGGPSVFLFPPKPKDTL
MISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPRVYTLPP [symbol] RDELTKNQVS
LTCLVKGFYPSDIAVEWESNGQPENNYKTTPPVLVSDGSFTLYSKLTVDKSRWQQG
NVFSCSVMHEALHNHYTQKSLSLSPG
Ab9 scFv (HC-LC)-Fc (Q347R, D399V, F405T, and S354C
substitutions)
[SEQ ID NO: 239]
EVQLVESGGGLVQPGGSLRLSCAASGFTFSSYWMSWVRQAPGK [symbol] LEWVANINTDGS
EVYYVDSVKGRFTISRDNAKNSLYLQMNSLRAEDTAVYYCARDVGPGIAYQGHFD
YWGQGTLVTVSSGGGGSGGGGSGGGGSGGGGSDIQMTQSPSSLSASVGDRVTITC
RASQVIYSYLNWYQQKPGKAPKLLIYAASSLKSGVPSRFSGSGSGTDFTLTISSLQPE
DFATYYCQQVYDTPLTFG [symbol] GTKVEIKASDKTHTCPPCPAPELLGGPSVFLFPPKPKDT
LMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPRVYTLPP [symbol] RDELTKNQV
SLTCLVKGFYPSDIAVEWESNGQPENNYKTTPPVLVSDGSFTLYSKLTVDKSRWQQG
NVFSCSVMHEALHNHYTQKSLSLSPG
Ab10 scFv (LC-HC)-Fc (Q347R, D399V, F405T, and S354C
substitutions)
[SEQ ID NO: 240]
EIVLTQSPATLSLSPGERATLSCRASHSVYSYLAWYQQKPGQAPRLLIYDASNRATGI
PARFSGSGSGTDFTLTISSLEPEDFAVYYCQQYDNLPTFG [symbol] GTKVEIKGGGGSGGGG
SGGGGSGGGGSQLQLQESGPGLVKPSETLSLTCTVSGGSISSTDYYWGWIRQPPGK [symbol]
LEWIGSIGYSGTYYNPSLKSRVTISVDTSKNQFSLKLSSVTAADTAVYYCARETAHDV
HGMDVWGQGTTVTVSSASDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDW
LNGKEYKCKVSNKALPAPIEKTISKAKGQPREPRVYTLPP [symbol] RDELTKNQVSLTCLVK
GFYPSDIAVEWESNGQPENNYKTTPPVLVSDGSFTLYSKLTVDKSRWQQGNVFSCSV
MHEALHNHYTQKSLSLSPG
Ab10 scFv (HC-LC)-Fc (Q347R, D399V, F405T, and S354C
substitutions)
[SEQ ID NO: 241]
QLQLQESGPGLVKPSETLSLTCTVSGGSISSTDYYWGWIRQPPGK [symbol] LEWIGSIGYSGT
YYNPSLKSRVTISVDTSKNQFSLKLSSVTAADTAVYYCARETAHDVHGMDVWGQG
TTVTVSSGGGGSGGGGSGGGGSGGGGSEIVLTQSPATLSLSPGERATLSCRASHSV
YSYLAWYQQKPGQAPRLLIYDASNRATGIPARFSGSGSGTDFTLTISSLEPEDFAVYY
CQQYDNLPTFG [symbol] GTKVEIKASDKTHTCPPCPAPELLGGPSVFLFPPKPKDTLMISRTPE
VTCVVVDVSHEDPEVKFNWYVDGVEVHNAKTKPREEQYNSTYRVVSVLTVLHQD
WLNGKEYKCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQGNVFSCS
VMHEALHNHYTQKSLSLSPG

A TriNKET of the present disclosure is A49-F3′-TriNKET-I07, comprising a first polypeptide chain, named “107 scFv-Fc,” which comprises a CD33-binding scFv linked to an Fc domain via an Ala-Ser linker; a second polypeptide chain, named “A49 VH-CH1-Fc,” which comprises an NKG2D-targeting heavy chain; and a third polypeptide chain, named “A49 VL-CL,” which comprises an NKG2D-targeting light chain. The amino acid sequence of 107 scFv-Fc comprises:

I07 scFv (LC-HC)-Fc (Q347R, D399V, F405T, and S354C
substitutions)
[SEQ ID NO: 187]
DIQMTQSPSTLSASVGDRVTITCRASQSISSWLAWYQQKPGKAPKLLIYE
ASSLESGVPSRFSGSGSGTEFTLTISSLQPDDFATYYCQQSQSYPPITFG [symbol]
GTKVEIKGGGGSGGGGSGGGGSGGGGSEVQLVESGGGLVQPGGSLRLSC
AASGFTFGSYWMSWVRQAPGK [symbol] LEWVATIKQDGSEKSYVDSVKGRFTISR
DNAKNSLYLQMNSLRAEDTAVYYCARPLNAGELDVWGQGTMVTVSSASDK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPRVYTLPP [symbol] RDELTKNQVSLTCLVKGF
YPSDIAVEWESNGQPENNYKTTPPVLVSDGSFTLYSKLTVDKSRWQQGNV
FSCSVMHEALHNHYTQKSLSLSPG

The amino acid sequence of the scFv portion of 107 scFv-Fc comprises:

I07 scFv
[SEQ ID NO: 188]
DIQMTQSPSTLSASVGDRVTITCRASQSISSWLAWYQQKPGKAPKLLIYE
ASSLESGVPSRFSGSGSGTEFTLTISSLQPDDFATYYCQQSQSYPPITFG [symbol]
GTKVEIKGGGGSGGGGSGGGGSGGGGSEVQLVESGGGLVQPGGSLRLSC
AASGFTFGSYWMSWVRQAPGK [symbol] LEWVATIKQDGSEKSYVDSVKGRFTISR
DNAKNSLYLQMNSLRAEDTAVYYCARPLNAGELDVWGQGTMVTVSS

A49 VH-CH1-Fc comprises A49 VH [SEQ ID NO:87] linked to a CH1 domain and an Fc domain (including hinge, CH2, and CH3 domains). The amino acid sequence of A49 VH-CH1-Fc comprises:

A49 VH-CH1-Fc (K360E, K409W, and Y349C
substitutions)
[SEQ ID NO: 189]
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQV [symbol] TLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G
A49 VH-CH1-Fc (Q347R, D399V, F405T, and S354C
substitutions)
[SEQ ID NO: 244]
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
RVYTLPP [symbol] RDELTKNQVSLTCLVKGFYPSDIAVEWESNGQPENNYKTTPP
VLVSDGSFTLYSKLTVDKSRWQQGNVFSCSVMHEALHNHYTQKSLSL

A49 VL-CL comprises A49 VL [SEQ ID NO:88] linked to a light chain constant domain (CL). The amino acid sequence of A49 VL-CL comprises:

A49 VL-CL
[SEQ ID NO: 190]
DIQMTQSPSSVSASVGDRVTITCRASQGISSWLAWYQQKPGKAPKLLIYAA
SSLQSGVPSRFSGSGSGTDFTLTISSLQPEDFATYYCQQGVSFPRTFGGFG
TKVEIKRTVAAPSVIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

Another TriNKET of the present disclosure is A49MI-F3′-TriNKET-I07, comprising a first polypeptide chain, named “107 scFv-Fc,” as described above [SEQ ID NO:187]; a second polypeptide chain, named “A49MI VH-CH1-Fc,” that comprises an NKG2D-targeting heavy chain with an Fc domain; and a third polypeptide chain, named “A49 VL-CL,” as described above [SEQ ID NO:190]. A49MI-F3′-TriNKET-I07 is identical to A49-F3′-TriNKET-I07 except for a substitution of I for M in CDR3 of the NKG2D-targeting VH. This substitution is bold-italic (and underlined because it is part of a CDR) in the sequence below. A49MI VH-CH1-Fc comprises A49MI VH [SEQ ID NO:191] linked to a CH1 domain and an Fc domain (including hinge, CH2, and CH3 domains). The amino acid sequence of A49MI VH-CH1-Fc comprises:

A49MI VH-CH1-Fc (K360E, K409W, and Y349C
substitutions)
[SEQ ID NO: 196]
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P [symbol] GAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQV [symbol] TLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G
A49MI VH-CH1-Fc (Q347R, D399V, F405T, and S354C
substitutions)
[SEQ ID NO: 245]
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P [symbol] GAAAGWFDPWGQGTLVTVSSASDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
RVYTLPP [symbol] RDELTKNQVSLTCLVKGFYPSDIAVEWESNGQPENNYKTTPP
VLVSDGSFTLYSKLTVDKSRWQQGNVFSCSVMHEALHNHYTQKSLSL

Another TriNKET of the present disclosure is A49-F3′-TriNKET-I07(si), comprising a first polypeptide chain, named “107 scFv-Fc(si),” which comprises a CD33-binding scFv linked to a silent Fc domain via an Ala-Ser linker; a second polypeptide chain, named “A49 VH-CH1-Fc(si),” which comprises an NKG2D-targeting heavy chain with a silent Fc domain; and a third polypeptide chain, named “A49 VL-CL,” as described above [SEQ ID NO: 190]. A49-F3′-TriNKET-I07(si) is identical to A49-F3′-TriNKET-I07 except for L234A, L235A, and P329A substitutions in both Fc domains. These substitutions are bold-italic in the two sequences below. The amino acid sequence of 107 scFv-Fc(si) comprises:

107 scFv-Fc(si)
[SEQ ID NO: 204]
DIQMTQSPSTLSASVGDRVTITCRASQSISSWLAWYQQKPGKAPKLLIYE
ASSLESGVPSRFSGSGSGTEFTLTISSLQPDDFATYYCQQSQSYPPITFG [symbol]
GTKVEIKGGGGSGGGGSGGGGSGGGGSEVQLVESGGGLVQPGGSLRLSC
AASGFTFGSYWMSWVRQAPGK [symbol] LEWVATIKQDGSEKSYVDSVKGRFTISR
RDNAKNSLYLQMNSLRAEDTAVYYCAPLNAGELDVWGQGTMVTVSSASDK
THTCPPCPAPE [symbol] GGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALGAPIEKTISKAKGQPREPRVYTLPP [symbol] RDELTKNQVSLTCLVKGF
YPSDIAVEWESNGQPENNYKTTPPVLVSDGSFTLYSKLTVDKSRWQQGNV
FSCSVMHEALHNHYTQKSLSLSPG

The amino acid sequence of the scFv portion of 107 scFv-Fc(si) is identical to that of the scFv portion of 107 scFv-Fc as described above [SEQ ID NO:188].

The amino acid sequence of A49 VH-CH1-Fc(si) comprises:

A49 VH-CH1-Fc(si)
[SEQ ID NO: 205]
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPE [symbol] GGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKAL [symbol] APIEKTISKAKGQPRE
PQV [symbol] TLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G

Another TriNKET of the present disclosure is A49-F3′-TriNKET-H76, comprising a first polypeptide chain, named “H76 scFv-Fc,” that comprises a CD33-binding scFv linked to an Fc domain via an Ala-Ser linker; a second polypeptide chain, named “A49 VH-CH1-Fc,” as described above [SEQ ID NO:189]; and a third polypeptide chain, named “A49 VL-CL,” as described above [SEQ ID NO:190]. The amino acid sequence of H76 scFv-Fc comprises:

H76 scFv-Fc
[SEQ ID NO: 197]
DIQMTQSPSTLSASVGDRVTITCRASQSISSWLAWYQQKPGKAPKLLIYK
ASSLESGVPSRFSGSGSGTEFTLTISSLQPDDFATYYCQQYDDLPTFG [symbol] G
TKVEIKGGGGSGGGGSGGGGSGGGGSEVQLLESGGGLVQPGGSLRLSCAA
SGFTFSKYTMSWVRQAPGK [symbol] LEWVSAIVGSGESTYFADSVKGRFTISRDN
SKNTLYLQMNSLRAEDTAVYYCAREGGPYYDSSGYFVYYGMDVWGQGTTV
TVSSASDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPRVYTLPP [symbol] RDELTKNQVS
LTCLVKGFYPSDIAVEWESNGQPENNYKTTPPVLVSDGSFTLYSKLTVDK
SRWQQGNVFSCSVMHEALHNHYTQKSLSLSPG

The amino acid sequence of the scFv portion of H76 scFv-Fc comprises:

H76 scFv
[SEQ ID NO: 198]
DIQMTQSPSTLSASVGDRVTITCRASQSISSWLAWYQQKPGKAPKLLIYK
ASSLESGVPSRFSGSGSGTEFTLTISSLQPDDFATYYCQQYDDLPTFG [symbol] G
TKVEIKGGGGSGGGGSGGGGSGGGGSEVQLLESGGGLVQPGGSLRLSCAA
SGFTFSKYTMSWVRQAPGK [symbol] LEWVSAIVGSGESTYFADSVKGRFTISRDN
SKNTLYLQMNSLRAEDTAVYYCAREGGPYYDSSGYFVYYGMDVWGQGTTV
TVSS

In other embodiments of the present disclosure, the CD33-binding scFv of the multi-specific binding protein (e.g., TriNKET) comprises the heavy chain variable domain CDR1, CDR2, and CDR3, and light chain variable domain CDR1, CDR2, and CDR3, of any one of the antibodies provided in Table 1. In certain embodiments, the amino acid sequence of the heavy chain variable domain is identical to the VH sequence of an antibody in Table 1 except for a substitution of Cys at position 44, and the amino acid sequence of the light chain variable domain is identical to the VL sequence of the same antibody except for a substitution of Cys at position 100. In certain embodiments, the heavy chain variable domain is connected to the light chain variable domain with a (G4S)₄ linker. The heavy chain variable domain can be N-terminal to the light chain variable domain or C-terminal to the light chain variable domain. The scFv is linked to an Fc domain via a hinge comprising Ala-Ser.

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the multi-specific binding proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

In certain embodiments, the antibody binds CD33 with a K_{D} of 20 nM, 15 nM, 10 nM, 9 nM, 8 nM, 7 nM, 6 nM, 5 nM, 4 nM, 3 nM, 2 nM, 1 nM or lower, as measured using standard binding assays, for example, surface plasmon resonance or bio-layer interferometry. In certain embodiments the antibody binds EBI3 from a body fluid, tissue and/or cell of a subject.

Competition assays for determining whether an antibody binds to the same epitope as, or competes for binding with a disclosed antibody, e.g., the Ab1 antibody, the Ab2 antibody, the Ab3 antibody, the Ab4 antibody, or the Ab5 antibody, are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human CD33 protein or fragment thereof) bound to a solid surface or expressed on a cell surface, a test CD33-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

In certain embodiments, the protein (e.g., multi-specific binding protein) of the present disclosure is not substantially internalized by a CD33-expressing cell. A low level of internalization may improve the pharmacokinetics of the protein, thereby reducing the dose required to engage CD33-expressing target cells with effector cells (e.g., NK cells). Internalization can be measured by any method known in the art, e.g., the methods described in Examples 5 and 10 of the present disclosure. For example, in certain embodiments, internalization of the protein (e.g., multi-specific binding protein) by EOL-1 cells is lower than 25%, 30%, 35%, 40%, 45%, or 50% after a two-hour incubation, as assessed by the methods disclosed herein. In certain embodiments, internalization of the protein (e.g., multi-specific binding protein) by EOL-1 cells is lower than 25%, 30%, 35%, 40%, 45%, or 50% after a 24-hour incubation, as assessed by the methods disclosed herein. In certain embodiments, internalization of the protein (e.g., multi-specific binding protein) by Molm-13 cells is lower than 25%, 30%, 35%, 40%, 45%, or 50% after a two-hour incubation, as assessed by the methods disclosed herein.

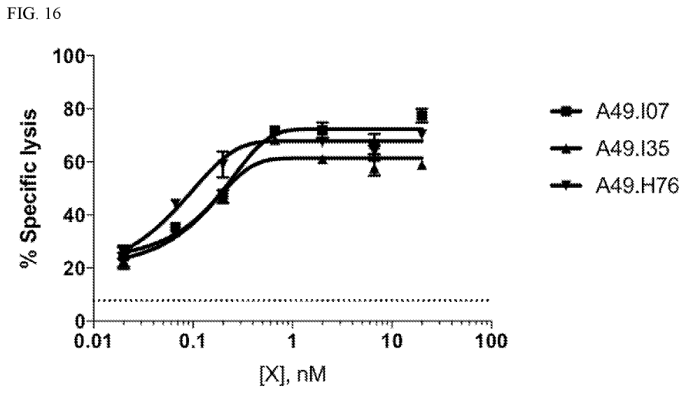

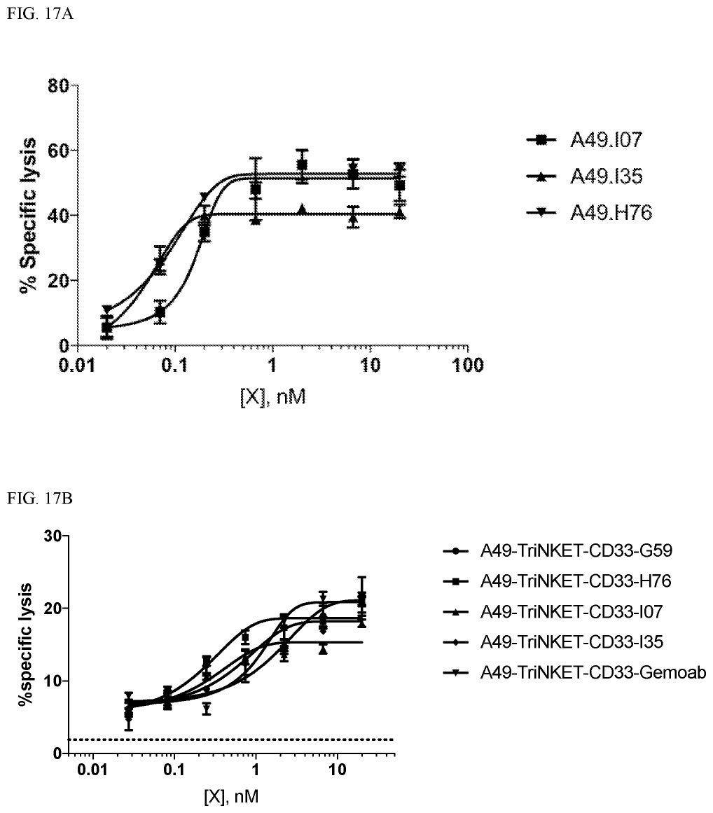

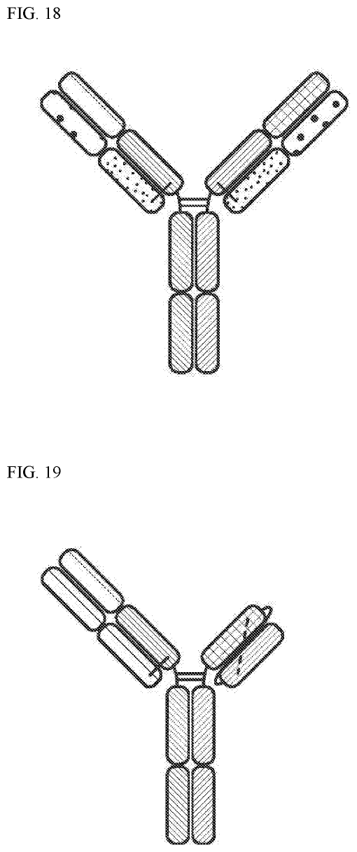

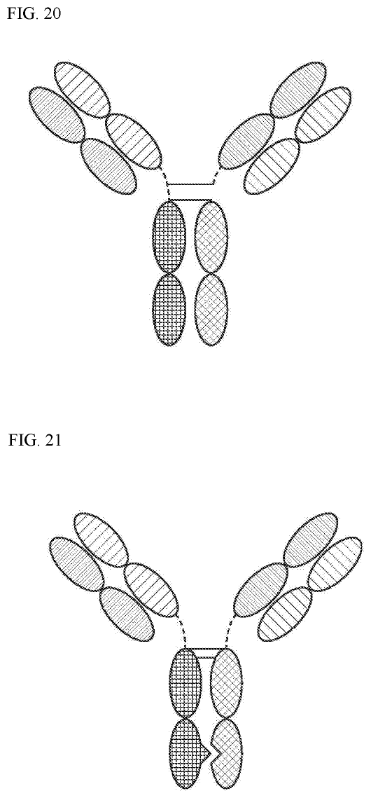

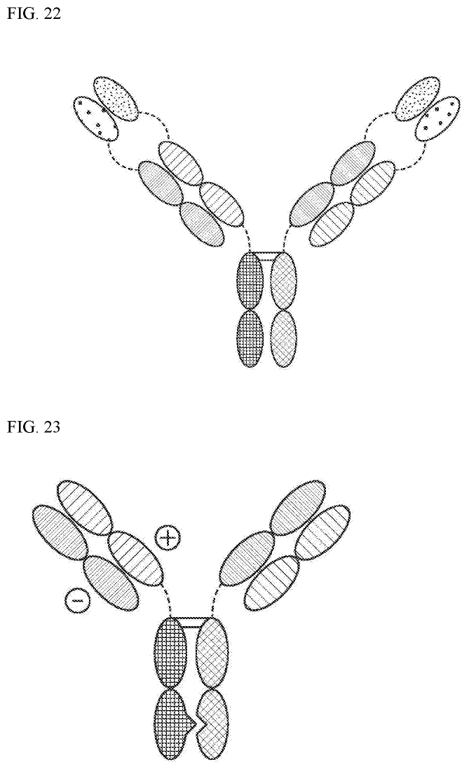

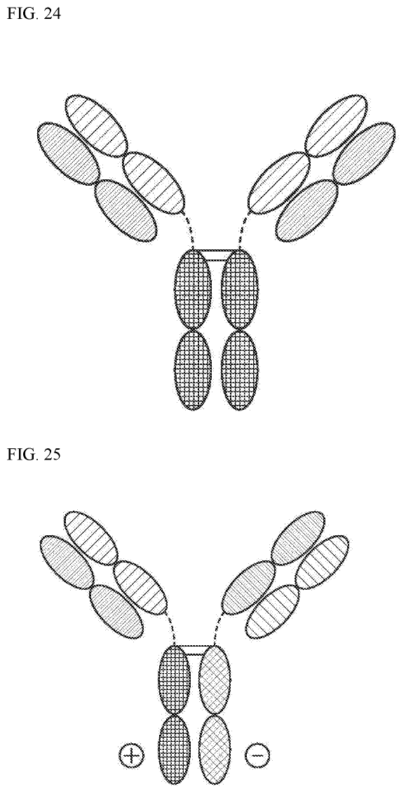

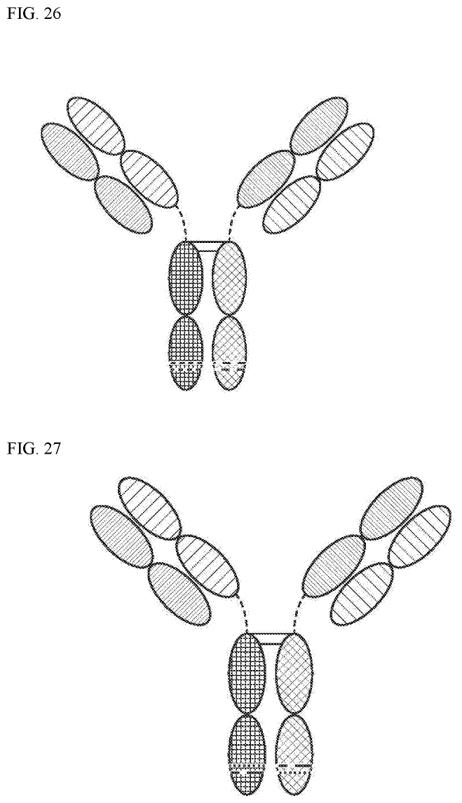

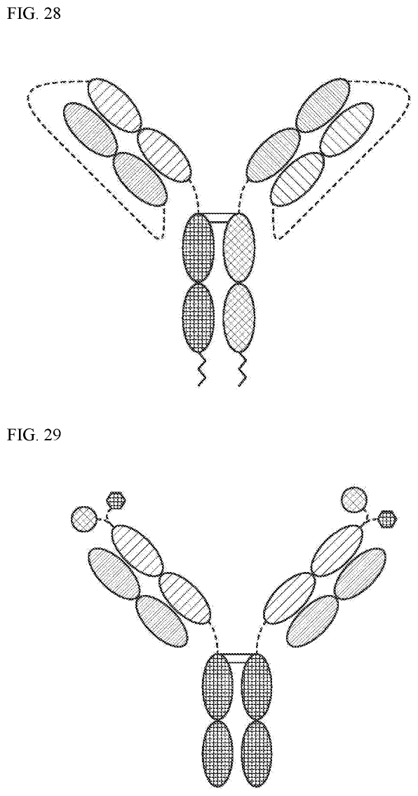

KHYG-1 cells express surface NKG2D, but do not express CD16. In certain embodiments, TriNKET mediated killings of Molm-13 and THP-1 cells are dependent upon NKG2D-mediated activation of the KHYG-1 effector cells. In certain embodiments, TriNKETs of the present disclosure mediate KHYG-1 effector cell killing of Molm-13 (FIG. 15A), EOL-1 (FIG. 16), and THP-1 (FIG. 17A) human AML target cell lines.

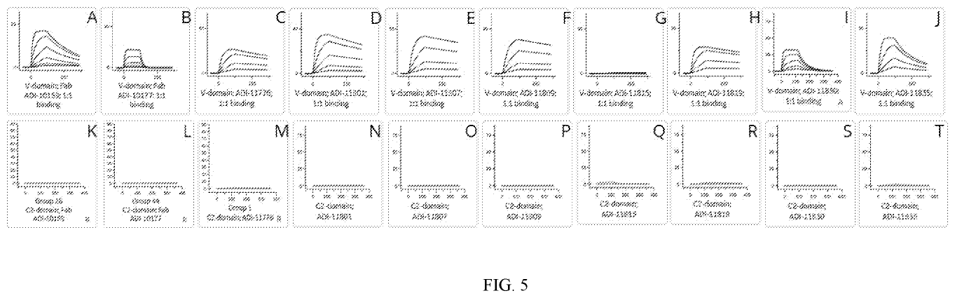

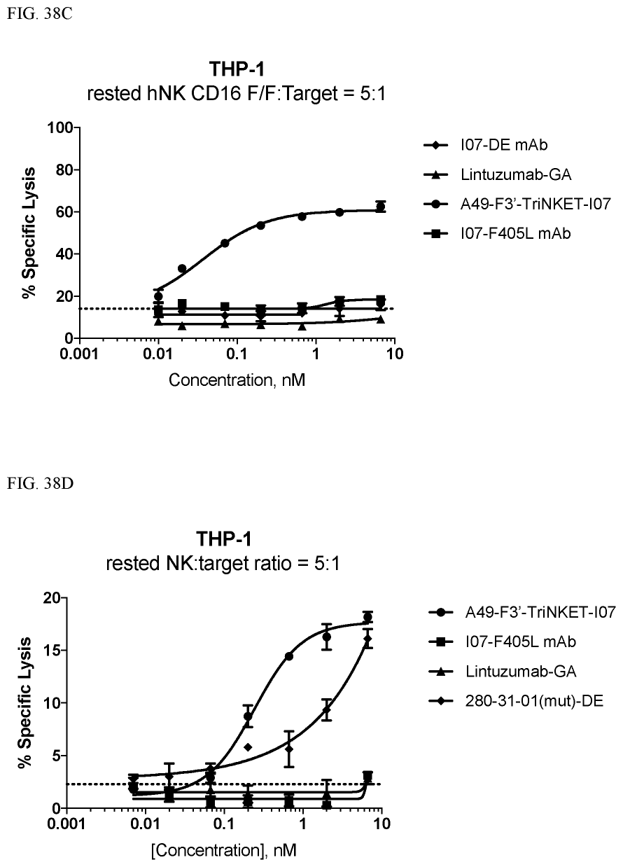

In certain embodiments, TriNKETs of the present disclosure mediate cytotoxicity of rested human NK cells against Molm-13 or THP-1 human AML cells. FIGS. 15B and 38A show that TriNKETs of the present disclosure mediate rested human NK cell killing of Molm-13 human AML cells. The rested human NK effector cell (E) to target cancer cell (T) ratio (E:T) was 10:1 in FIG. 15B and 5:1 in FIG. 38A. The E:T ratio may reflect differences in the maximal % lysis.

In certain embodiments, TriNKETs of the present disclosure mediate rested human NK cell killing of EOL-1 human AML cells. FIG. 38B shows that TriNKETs mediate rested human NK cell killing of EOL-1 cells at an E:T of 5:1. In certain embodiments, TriNKETs of the present disclosure mediate rested human NK cell killing of THP-1 target cells, which express the high-affinity FcγRI. FIGS. 17B, 38C, and 38D show that TriNKETs mediate rested human NK cell killing of THP-1 human AML cells using an E:T of 5:1.

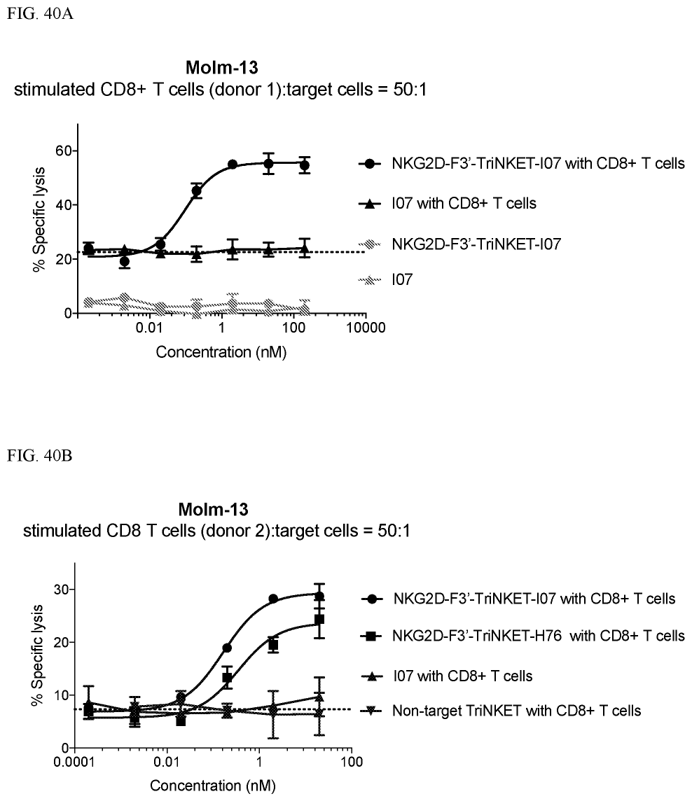

In certain embodiments, TriNKETs of the present disclosure mediate human CD8⁺ T cell killing of Molm-13 target cells. FIGS. 40A-40B show that TriNKETs mediate human CD8⁺ T cell killing of Molm-13 cells at an E:T of 50:1.

A Protein Comprising an Antigen-Binding Site that Competes with the CD33-Binding Sites Described Herein

In one aspect, the present invention provides a protein that includes an antigen-binding site that competes with the CD33-binding sites described herein to bind to CD33. In certain embodiments, the present invention provides a protein that includes an antigen-binding site that competes for binding to human and cynomolgus CD33 with an antibody that includes an antibody heavy chain having the amino acid sequence of SEQ ID NO:1 and an antibody light chain having the amino acid sequence of SEQ ID NO:2.

In certain embodiments, the present invention provides a protein that includes an antigen-binding site that competes for binding to human and cynomolgus CD33 with an antibody that includes an antibody heavy chain having the amino acid sequence of SEQ ID NO:3 and an antibody light chain having the amino acid sequence of SEQ ID NO:4.

In certain embodiments, the present invention provides a protein that includes an antigen-binding site that competes for binding to human and cynomolgus CD33 with an antibody that includes an antibody heavy chain having the amino acid sequence of SEQ ID NO:5 and an antibody light chain having the amino acid sequence of SEQ ID NO:6.

In certain embodiments, the present invention provides a protein that includes an antigen-binding site that competes for binding to human and cynomolgus CD33 with an antibody that includes an antibody heavy chain having the amino acid sequence of SEQ ID NO:7 and an antibody light chain having the amino acid sequence of SEQ ID NO:8.

In certain embodiments, the present invention provides a protein that includes an antigen-binding site that competes for binding to human and cynomolgus CD33 with an antibody that includes an antibody heavy chain having the amino acid sequence of SEQ ID NO:9 and an antibody light chain having the amino acid sequence of SEQ ID NO:10.

In certain embodiments, the present invention provides a protein that includes an antigen-binding site that competes for binding to human and cynomolgus CD33 with an antibody that includes an antibody heavy chain having the amino acid sequence of SEQ ID NO:11 and an antibody light chain having the amino acid sequence of SEQ ID NO:12.

In certain embodiments, the present invention provides a protein that includes an antigen-binding site that competes for binding to human and cynomolgus CD33 with an antibody that includes an antibody heavy chain having the amino acid sequence of SEQ ID NO:13 and an antibody light chain having the amino acid sequence of SEQ ID NO:14.

In certain embodiments, the present invention provides a protein that includes an antigen-binding site that competes for binding to human and cynomolgus CD33 with an antibody that includes an antibody heavy chain having the amino acid sequence of SEQ ID NO:15 and an antibody light chain having the amino acid sequence of SEQ ID NO:16.

In certain embodiments, the present invention provides a protein that includes an antigen-binding site that competes for binding to human and cynomolgus CD33 with an antibody that includes an antibody heavy chain having the amino acid sequence of SEQ ID NO:17 and an antibody light chain having the amino acid sequence of SEQ ID NO:18.

In certain embodiments, the present invention provides a protein that includes an antigen-binding site that competes for binding to human and cynomolgus CD33 with an antibody that includes an antibody heavy chain having the amino acid sequence of SEQ ID NO:19 and an antibody light chain having the amino acid sequence of SEQ ID NO:20.

In some embodiments, an antigen-binding site of the protein that competes with the CD33-binding sites includes a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:1 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:2. In some embodiments, an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:3 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:4.

In some embodiments, an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:5 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:6.

In some embodiments, an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:7 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:8.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:9 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:10.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:11 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:12.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:13 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:14.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:15 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:16.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:17 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:18.

In some embodiments an antigen-binding site of the protein that competes with the CD33-binding sites includes a heavy chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:19 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:20.

In certain embodiments, the present invention provides a protein that includes an antigen-binding site that competes for binding to human and cynomolgus CD33 with an antibody that includes an antigen-binding site that binds a tumor-associated antigen.

In certain embodiments, the present invention provides a protein that includes an antigen-binding site that competes for binding to human and cynomolgus CD33 with an antibody that includes an antibody constant region or a portion thereof capable of binding CD16.

### CAR T Cells, CD33/CD3-Directed Bispecific T-Cell Engagers, Immunocytokines, Antibody-Drug Conjugates, and Immunotoxins

Another aspect of the present invention provides a molecule or complex comprising an antigen-binding site that binds CD33 as disclosed herein. Exemplary molecules or complexes include but are not limited to chimeric antigen receptors (CARs), T-cell engagers (e.g., CD33/CD3-directed bispecific T-cell engagers), immunocytokines, antibody-drug conjugates, and immunotoxins.

Any antigen-binding site that binds CD33 as disclosed herein can be used, including but not limited to the antigen-binding site that binds CD33 as disclosed in Section I. Antigen-Binding Site. In certain embodiments, the amino acid sequence(s) of the antigen-binding site that binds CD33 are provided in Table 1. In certain embodiments, the antigen-binding site that binds CD33 is an scFv. In certain embodiments, the scFv comprises an amino acid sequence at least 90% (e.g., 90%, 91%, 92%, 93%, 94%, 95%, 96%, 97%, 98%, 99%, or 100%) identical to an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484. In certain embodiments, the scFv comprises an amino acid sequence selected from SEQ ID NO:188, SEQ ID NO:198, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, and SEQ ID NO:484.

In certain embodiments, the antigen-binding site that binds CD33 comprises a heavy chain variable domain comprising CDR1, CDR2, and CDR3 sequences represented by the amino acid sequences of SEQ ID NOs:181, 46, and 182, respectively; and a light chain variable domain comprising CDR1, CDR2, and CDR3 sequences represented by the amino acid sequences of SEQ ID NOs:48, 49, and 50, respectively. In certain embodiments, the antigen-binding site comprises a heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:9; and a light chain variable domain with an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the antigen-binding site comprises an scFv comprising an amino acid sequence at least 90% (e.g., 90%, 91%, 92%, 93%, 94%, 95%, 96%, 97%, 98%, 99%, or 100%) identical to SEQ ID NO:188.

In other embodiments of any one of the foregoing aspects in this subsection, the antigen-binding site that binds CD33 comprises a heavy chain variable domain comprising CDR1, CDR2, and CDR3 sequences represented by the amino acid sequences of SEQ ID NOs:183, 34, and 184, respectively; and a light chain variable domain comprising CDR1, CDR2, and CDR3 sequences represented by the amino acid sequences of SEQ ID NOs:36, 185, and 38, respectively. In certain embodiments, the antigen-binding site comprises a heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:5; and a light chain variable domain with an amino acid sequence at least 90% (e.g., 91%, 92%, 93%, 94%, 95%, 96%, 97%, 98%, 99%, or 100%) identical to the amino acid sequence of SEQ ID NO:6. In certain embodiments, the antigen-binding site comprises an scFv comprising an amino acid sequence at least 90% (e.g., 90%, 91%, 92%, 93%, 94%, 95%, 96%, 97%, 98%, 99%, or 100%) identical to SEQ ID NO:198.

### Chimeric Antigen Receptors (CARs)

In certain embodiments, the present invention provides a CD33-targeting CAR comprising an antigen-binding site that binds CD33 as disclosed herein (see, e.g., Table 1). The CD33-targeting CAR can comprise an Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds CD33 as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In one embodiment, the CAR comprises a chimeric fusion protein comprising a CD33-binding domain (e.g., CD33-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising a primary signaling domain. In one embodiment, the CAR comprises a chimeric fusion protein comprising a CD33-binding domain (e.g., CD33-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain and an intracellular signaling domain comprising a costimulatory signaling domain and a primary signaling domain. In one aspect, the CAR comprises a chimeric fusion protein comprising a CD33-binding domain (e.g., CD33-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising two costimulatory signaling domains and a primary signaling domain. In one embodiment, the CAR comprises a chimeric fusion protein comprising a CD33-binding domain comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 434, 22, and 435, respectively, and a light chain variable domain comprising CDR1, CDR2, and CDR3 having the amino acid sequences of SEQ ID NOs: 24, 25, and 26, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:1, and the light chain variable domain comprises an amino acid sequence at least 90% (e.g., 90%, 91%, 92%, 93%, 94%, 95%, 96%, 97%, 98%, 99%, or 100%) identical to SEQ ID NO:2. In certain embodiments, the extracellular antigen binding domain comprises an scFv having the amino acid sequence of SEQ ID NO:206 or SEQ ID NO:207.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 181, 28, and 436, respectively, and a light chain variable domain comprising CDR1, CDR2, and CDR3 having the amino acid sequences of SEQ ID NOs: 30, 31, and 32, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:3, and the light chain variable domain comprises an amino acid sequence at least 90% (e.g., 90%, 91%, 92%, 93%, 94%, 95%, 96%, 97%, 98%, 99%, or 100%) identical to SEQ ID NO:4. In certain embodiments, the extracellular antigen binding domain comprises an scFv having the amino acid sequence of SEQ ID NO:208 or SEQ ID NO:209.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 183, 34, and 184, respectively, and a light chain variable domain comprising CDR1, CDR2, and CDR3 having the amino acid sequences of SEQ ID NOs: 36, 37, and 38, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:5, and the light chain variable domain comprises an amino acid sequence at least 90% (e.g., 90%, 91%, 92%, 93%, 94%, 95%, 96%, 97%, 98%, 99%, or 100%) identical to SEQ ID NO:6. In certain embodiments, the extracellular antigen binding domain comprises an scFv having the amino acid sequence of SEQ ID NO:198 or SEQ ID NO:210.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 437, 40, and 438, respectively, and a light chain variable domain comprising CDR1, CDR2, and CDR3 having the amino acid sequences of SEQ ID NOs: 42, 43, and 44, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:7, and the light chain variable domain comprises an amino acid sequence at least 90% (e.g., 90%, 91%, 92%, 93%, 94%, 95%, 96%, 97%, 98%, 99%, or 100%) identical to SEQ ID NO:8. In certain embodiments, the extracellular antigen binding domain comprises an scFv having the amino acid sequence of SEQ ID NO:211 or SEQ ID NO:212.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 181, 46, and 182, respectively, and a light chain variable domain comprising CDR1, CDR2, and CDR3 having the amino acid sequences of SEQ ID NOs: 48, 49, and 50, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:9, and the light chain variable domain comprises an amino acid sequence at least 90% (e.g., 90%, 91%, 92%, 93%, 94%, 95%, 96%, 97%, 98%, 99%, or 100%) identical to SEQ ID NO:10. In certain embodiments, the extracellular antigen binding domain comprises an scFv having the amino acid sequence of SEQ ID NO:188 or SEQ ID NO:213.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 181, 52, and 439, respectively, and a light chain variable domain comprising CDR1, CDR2, and CDR3 having the amino acid sequences of SEQ ID NOs: 54, 55, and 56, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:11, and the light chain variable domain comprises an amino acid sequence at least 90% (e.g., 90%, 91%, 92%, 93%, 94%, 95%, 96%, 97%, 98%, 99%, or 100%) identical to SEQ ID NO:12. In certain embodiments, the extracellular antigen binding domain comprises an scFv having the amino acid sequence of SEQ ID NO:214 or SEQ ID NO:215.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 440, 58, and 441, respectively, and a light chain variable domain comprising CDR1, CDR2, and CDR3 having the amino acid sequences of SEQ ID NOs: 60, 61, and 62, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:13, and the light chain variable domain comprises an amino acid sequence at least 90% (e.g., 90%, 91%, 92%, 93%, 94%, 95%, 96%, 97%, 98%, 99%, or 100%) identical to SEQ ID NO:14. In certain embodiments, the extracellular antigen binding domain comprises an scFv having the amino acid sequence of SEQ ID NO:216 or SEQ ID NO:217.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 442, 64, and 443, respectively, and a light chain variable domain comprising CDR1, CDR2, and CDR3 having the amino acid sequences of SEQ ID NOs: 66, 67, and 68, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:15, and the light chain variable domain comprises an amino acid sequence at least 90% (e.g., 90%, 91%, 92%, 93%, 94%, 95%, 96%, 97%, 98%, 99%, or 100%) identical to SEQ ID NO:16. In certain embodiments, the extracellular antigen binding domain comprises an scFv having the amino acid sequence of SEQ ID NO:218 or SEQ ID NO:219.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 181, 70, and 444, respectively, and a light chain variable domain comprising CDR1, CDR2, and CDR3 having the amino acid sequences of SEQ ID NOs: 72, 73, and 74, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:17, and the light chain variable domain comprises an amino acid sequence at least 90% (e.g., 90%, 91%, 92%, 93%, 94%, 95%, 96%, 97%, 98%, 99%, or 100%) identical to SEQ ID NO:18. In certain embodiments, the extracellular antigen binding domain comprises an scFv having the amino acid sequence of SEQ ID NO:220 or SEQ ID NO:221.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 445, 76, and 446, respectively, and a light chain variable domain comprising CDR1, CDR2, and CDR3 having the amino acid sequences of SEQ ID NOs: 78, 79, and 80, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:19, and the light chain variable domain comprises an amino acid sequence at least 90% (e.g., 90%, 91%, 92%, 93%, 94%, 95%, 96%, 97%, 98%, 99%, or 100%) identical to SEQ ID NO:20. In certain embodiments, the extracellular antigen binding domain comprises an scFv having the amino acid sequence of SEQ ID NO:222 or SEQ ID NO:223.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 528, 305, and 529, respectively, and a light chain variable domain comprising CDR1, CDR2, and CDR3 having the amino acid sequences of SEQ ID NOs: 307, 308, and 309, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:266, and the light chain variable domain comprises an amino acid sequence at least 90% (e.g., 90%, 91%, 92%, 93%, 94%, 95%, 96%, 97%, 98%, 99%, or 100%) identical to SEQ ID NO:267. In certain embodiments, the extracellular antigen binding domain comprises an scFv having the amino acid sequence of SEQ ID NO:447 or SEQ ID NO:448.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 530, 311, and 531, respectively, and a light chain variable domain comprising CDR1, CDR2, and CDR3 having the amino acid sequences of SEQ ID NOs: 313, 314, and 315, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:268, and the light chain variable domain comprises an amino acid sequence at least 90% (e.g., 90%, 91%, 92%, 93%, 94%, 95%, 96%, 97%, 98%, 99%, or 100%) identical to SEQ ID NO:269. In certain embodiments, the extracellular antigen binding domain comprises an scFv having the amino acid sequence of SEQ ID NO:449 or SEQ ID NO:450.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 532, 317, and 533, respectively, and a light chain variable domain comprising CDR1, CDR2, and CDR3 having the amino acid sequences of SEQ ID NOs: 319, 320, and 321, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:270, and the light chain variable domain comprises an amino acid sequence at least 90% (e.g., 90%, 91%, 92%, 93%, 94%, 95%, 96%, 97%, 98%, 99%, or 100%) identical to SEQ ID NO:271. In certain embodiments, the extracellular antigen binding domain comprises an scFv having the amino acid sequence of SEQ ID NO:451 or SEQ ID NO:452.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 534, 323, and 535, respectively, and a light chain variable domain comprising CDR1, CDR2, and CDR3 having the amino acid sequences of SEQ ID NOs: 325, 326, and 327, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:272, and the light chain variable domain comprises an amino acid sequence at least 90% (e.g., 90%, 91%, 92%, 93%, 94%, 95%, 96%, 97%, 98%, 99%, or 100%) identical to SEQ ID NO:273. In certain embodiments, the extracellular antigen binding domain comprises an scFv having the amino acid sequence of SEQ ID NO:453 or SEQ ID NO:454.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 536, 329, and 537, respectively, and a light chain variable domain comprising CDR1, CDR2, and CDR3 having the amino acid sequences of SEQ ID NOs: 331, 332, and 333, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:274, and the light chain variable domain comprises an amino acid sequence at least 90% (e.g., 90%, 91%, 92%, 93%, 94%, 95%, 96%, 97%, 98%, 99%, or 100%) identical to SEQ ID NO:275. In certain embodiments, the extracellular antigen binding domain comprises an scFv having the amino acid sequence of SEQ ID NO:455 or SEQ ID NO:456.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 538, 335, and 539, respectively, and a light chain variable domain comprising CDR1, CDR2, and CDR3 having the amino acid sequences of SEQ ID NOs: 337, 338, and 339, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:276, and the light chain variable domain comprises an amino acid sequence at least 90% (e.g., 90%, 91%, 92%, 93%, 94%, 95%, 96%, 97%, 98%, 99%, or 100%) identical to SEQ ID NO:277. In certain embodiments, the extracellular antigen binding domain comprises an scFv having the amino acid sequence of SEQ ID NO:457 or SEQ ID NO:458.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 540, 341, and 541, respectively, and a light chain variable domain comprising CDR1, CDR2, and CDR3 having the amino acid sequences of SEQ ID NOs: 343, 344, and 345, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:278, and the light chain variable domain comprises an amino acid sequence at least 90% (e.g., 90%, 91%, 92%, 93%, 94%, 95%, 96%, 97%, 98%, 99%, or 100%) identical to SEQ ID NO:279. In certain embodiments, the extracellular antigen binding domain comprises an scFv having the amino acid sequence of SEQ ID NO:459 or SEQ ID NO:460.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 542, 347, and 543, respectively, and a light chain variable domain comprising CDR1, CDR2, and CDR3 having the amino acid sequences of SEQ ID NOs: 349, 350, and 351, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:280, and the light chain variable domain comprises an amino acid sequence at least 90% (e.g., 90%, 91%, 92%, 93%, 94%, 95%, 96%, 97%, 98%, 99%, or 100%) identical to SEQ ID NO:281. In certain embodiments, the extracellular antigen binding domain comprises an scFv having the amino acid sequence of SEQ ID NO:461 or SEQ ID NO:462.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 544, 353, and 545, respectively, and a light chain variable domain comprising CDR1, CDR2, and CDR3 having the amino acid sequences of SEQ ID NOs: 355, 356, and 357, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:282, and the light chain variable domain comprises an amino acid sequence at least 90% (e.g., 90%, 91%, 92%, 93%, 94%, 95%, 96%, 97%, 98%, 99%, or 100%) identical to SEQ ID NO:283. In certain embodiments, the extracellular antigen binding domain comprises an scFv having the amino acid sequence of SEQ ID NO:463 or SEQ ID NO:464.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 546, 359, and 547, respectively, and a light chain variable domain comprising CDR1, CDR2, and CDR3 having the amino acid sequences of SEQ ID NOs: 361, 362, and 363, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:284, and the light chain variable domain comprises an amino acid sequence at least 90% (e.g., 90%, 91%, 92%, 93%, 94%, 95%, 96%, 97%, 98%, 99%, or 100%) identical to SEQ ID NO:285. In certain embodiments, the extracellular antigen binding domain comprises an scFv having the amino acid sequence of SEQ ID NO:465 or SEQ ID NO:466.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 548, 365, and 549, respectively, and a light chain variable domain comprising CDR1, CDR2, and CDR3 having the amino acid sequences of SEQ ID NOs: 367, 368, and 369, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:286, and the light chain variable domain comprises an amino acid sequence at least 90% (e.g., 90%, 91%, 92%, 93%, 94%, 95%, 96%, 97%, 98%, 99%, or 100%) identical to SEQ ID NO:287. In certain embodiments, the extracellular antigen binding domain comprises an scFv having the amino acid sequence of SEQ ID NO:467 or SEQ ID NO:468.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 550, 371, and 551, respectively, and a light chain variable domain comprising CDR1, CDR2, and CDR3 having the amino acid sequences of SEQ ID NOs: 373, 374, and 375, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:288, and the light chain variable domain comprises an amino acid sequence at least 90% (e.g., 90%, 91%, 92%, 93%, 94%, 95%, 96%, 97%, 98%, 99%, or 100%) identical to SEQ ID NO:289. In certain embodiments, the extracellular antigen binding domain comprises an scFv having the amino acid sequence of SEQ ID NO:469 or SEQ ID NO:470.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 552, 377, and 553, respectively, and a light chain variable domain comprising CDR1, CDR2, and CDR3 having the amino acid sequences of SEQ ID NOs: 379, 380, and 381, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:290, and the light chain variable domain comprises an amino acid sequence at least 90% (e.g., 90%, 91%, 92%, 93%, 94%, 95%, 96%, 97%, 98%, 99%, or 100%) identical to SEQ ID NO:291. In certain embodiments, the extracellular antigen binding domain comprises an scFv having the amino acid sequence of SEQ ID NO:471 or SEQ ID NO:472.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 554, 383, and 555, respectively, and a light chain variable domain comprising CDR1, CDR2, and CDR3 having the amino acid sequences of SEQ ID NOs: 385, 386, and 387, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:292, and the light chain variable domain comprises an amino acid sequence at least 90% (e.g., 90%, 91%, 92%, 93%, 94%, 95%, 96%, 97%, 98%, 99%, or 100%) identical to SEQ ID NO:293. In certain embodiments, the extracellular antigen binding domain comprises an scFv having the amino acid sequence of SEQ ID NO:473 or SEQ ID NO:474.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 556, 389, and 557, respectively, and a light chain variable domain comprising CDR1, CDR2, and CDR3 having the amino acid sequences of SEQ ID NOs: 391, 392, and 393, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:294, and the light chain variable domain comprises an amino acid sequence at least 90% (e.g., 90%, 91%, 92%, 93%, 94%, 95%, 96%, 97%, 98%, 99%, or 100%) identical to SEQ ID NO:295. In certain embodiments, the extracellular antigen binding domain comprises an scFv having the amino acid sequence of SEQ ID NO:475 or SEQ ID NO:476.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 558, 395, and 559, respectively, and a light chain variable domain comprising CDR1, CDR2, and CDR3 having the amino acid sequences of SEQ ID NOs: 397, 398, and 399, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:296, and the light chain variable domain comprises an amino acid sequence at least 90% (e.g., 90%, 91%, 92%, 93%, 94%, 95%, 96%, 97%, 98%, 99%, or 100%) identical to SEQ ID NO:297. In certain embodiments, the extracellular antigen binding domain comprises an scFv having the amino acid sequence of SEQ ID NO:477 or SEQ ID NO:478.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 560, 401, and 561, respectively, and a light chain variable domain comprising CDR1, CDR2, and CDR3 having the amino acid sequences of SEQ ID NOs: 403, 404, and 405, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:298, and the light chain variable domain comprises an amino acid sequence at least 90% (e.g., 90%, 91%, 92%, 93%, 94%, 95%, 96%, 97%, 98%, 99%, or 100%) identical to SEQ ID NO:299. In certain embodiments, the extracellular antigen binding domain comprises an scFv having the amino acid sequence of SEQ ID NO:479 or SEQ ID NO:480.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 562, 407, and 563, respectively, and a light chain variable domain comprising CDR1, CDR2, and CDR3 having the amino acid sequences of SEQ ID NOs: 409, 410, and 411, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:300, and the light chain variable domain comprises an amino acid sequence at least 90% (e.g., 90%, 91%, 92%, 93%, 94%, 95%, 96%, 97%, 98%, 99%, or 100%) identical to SEQ ID NO:301. In certain embodiments, the extracellular antigen binding domain comprises an scFv having the amino acid sequence of SEQ ID NO:481 or SEQ ID NO:482.

In certain embodiments, the extracellular antigen binding domain comprises an antigen-binding site (e.g., an scFv) comprising a heavy chain variable domain comprising CDR1, CDR2, and CDR3 having the amino acid sequences of SEQ ID NOs: 564, 413, and 565, respectively, and a light chain variable domain comprising CDR1, CDR2, and CDR3 having the amino acid sequences of SEQ ID NOs: 415, 416, and 417, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:302, and the light chain variable domain comprises an amino acid sequence at least 90% (e.g., 90%, 91%, 92%, 93%, 94%, 95%, 96%, 97%, 98%, 99%, or 100%) identical to SEQ ID NO:303. In certain embodiments, the extracellular antigen binding domain comprises an scFv having the amino acid sequence of SEQ ID NO:483 or SEQ ID NO:484.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR a chain, TCR 0 chain, TCR chain, CD28, CD3c, CD45, CD4, CDS, CD8, CD9, CD16, CD22, CD33, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Ry, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular CD33-binding domain (e.g., CD33-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker, a KIR2DS2 hinge, and a CD8a hinge.

The intracellular signaling domain of the CAR of the invention is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MEW molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the invention include those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, the primary signaling domain in any one or more CARs of the invention comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CDS, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR of the invention may be linked to each other in a random or specified order. Optionally, a short oligo- or polypeptide linker, for example, between 2 and 10 amino acids in length may form the linkage.

Another aspect of the present invention provides a nucleic acid encoding a CD33-targeting CAR disclosed herein. The nucleic acid is useful for expressing the CAR in an effector cell (e.g., T cell) by introducing the nucleic acid to the cell.

Exemplary nucleic acid sequences encoding the extracellular portions of the CARs are disclosed herein, e.g., as SEQ ID NOs:246-265. Modifications may be made in the sequence to create an equivalent or improved variant of the invention, for example, by changing one or more of the codons according to the codon degeneracy table. A DNA codon degeneracy table is provided in Table 10.

TABLE 10
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG CGU |
| Serine | S | Ser | AGC AGU UCA UCC UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present invention provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular CD33-binding domain (e.g., CD33-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

Another aspect of the present invention provides an immune effector cell expressing the CD33-targeting CAR. Also provided is an immune effector cell comprising the nucleic acid encoding the CD33-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present invention provides a pluripotent or multipotent cell (e.g., a hematopoietic stem cell) expressing the CD33-targeting CAR or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present invention is a cell obtained from in vitro expansion.

Further embodiments of the CD33-targeting CAR (e.g., regulatable CAR), nucleic acid encoding the CAR, and effector cells expressing the CAR or comprising the nucleic acid are provided in U.S. Pat. Nos. 7,446,190 and 9,181,527, U.S. Patent Application Publication Nos. 2016/0340406 and 2017/0049819, and International Patent Application Publication No. WO2018/140725.

### CD33/CD3-Directed Bispecific T-Cell Engagers

In certain embodiments, the present invention provides a CD33/CD3-directed bispecific T-cell engager comprising an antigen-binding site that binds CD33 disclosed herein. In certain embodiments, the CD33/CD3-directed bispecific T-cell engager comprises an amino acid sequence at least 90% (e.g., 90%, 91%, 92%, 93%, 94%, 95%, 96%, 97%, 98%, 99%, or 100%) identical to SEQ ID NO:187. In certain embodiments, the CD33/CD3-directed bispecific T-cell engager comprises an amino acid sequence at least 90% (e.g., 90%, 91%, 92%, 93%, 94%, 95%, 96%, 97%, 98%, 99%, or 100%) identical to SEQ ID NO:197.

In certain embodiments, the CD33/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present invention provides a nucleic acid encoding at least one polypeptide of the CD33/CD3-directed bispecific T-cell engager, wherein the polypeptide comprises an antigen binding site that binds CD33. In certain embodiments, the nucleic acid further comprises a nucleotide sequence encoding a signal peptide that, when expressed, is at the N-terminus of one or more of the polypeptides of the CD33/CD3-directed bispecific T-cell engager. Also provided is a vector (e.g., a viral vector) comprising the nucleic acid, a producer cell comprising the nucleic acid or vector, and a producer cell expressing the CD33/CD3-directed bispecific T-cell engager.

### Immunocytokines

In certain embodiments, the present invention provides an immunocytokine comprising an antigen-binding site that binds CD33 disclosed herein and a cytokine. Any cytokine (e.g., pro-inflammatory cytokines) known in the art can be used, including but not limited to IL-2, IL-4, IL-10, IL-12, IL-15, TNF, IFNα, IFNα, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of polypeptide. The immunocytokine can further comprise an Fc domain connected to the antigen-binding site that binds CD33. In certain embodiments, the immunocytokine comprises an amino acid sequence at least 90% (e.g., 90%, 91%, 92%, 93%, 94%, 95%, 96%, 97%, 98%, 99%, or 100%) identical to SEQ ID NO:187. In certain embodiments, the immunocytokine comprises an amino acid sequence at least 90% (e.g., 90%, 91%, 92%, 93%, 94%, 95%, 96%, 97%, 98%, 99%, or 100%) identical to SEQ ID NO:197. In certain embodiments, the cytokine is connected to the Fc domain directly or via a linker.

Another aspect of the present invention provides a nucleic acid encoding at least one polypeptide of the immunocytokine, wherein the polypeptide comprises an antigen binding site that binds CD33. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present invention provides an antibody-drug conjugate comprising an antigen-binding site that binds CD33 disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds CD33. In certain embodiments, the antibody-drug conjugate comprises an amino acid sequence at least 90% (e.g., 90%, 91%, 92%, 93%, 94%, 95%, 96%, 97%, 98%, 99%, or 100%) identical to SEQ ID NO:187. In certain embodiments, the antibody-drug conjugate comprises an amino acid sequence at least 90% (e.g., 90%, 91%, 92%, 93%, 94%, 95%, 96%, 97%, 98%, 99%, or 100%) identical to SEQ ID NO:197. In certain embodiments, the cytotoxic drug moiety is connected to the Fc domain directly or via a linker.

### Immunotoxins

In certain embodiments, the present invention provides an immunotoxin comprising an antigen-binding site that binds CD33 disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds CD33. In certain embodiments, the immunotoxin comprises an amino acid sequence at least 90% (e.g., 90%, 91%, 92%, 93%, 94%, 95%, 96%, 97%, 98%, 99%, or 100%) identical to SEQ ID NO:187. In certain embodiments, the immunotoxin comprises an amino acid sequence at least 90% (e.g., 90%, 91%, 92%, 93%, 94%, 95%, 96%, 97%, 98%, 99%, or 100%) identical to SEQ ID NO:197. In certain embodiments, the cytotoxic peptide moiety is connected to the Fc domain directly or via a linker.

Another aspect of the present invention provides a nucleic acid encoding at least one polypeptide of the immunotoxin, wherein the polypeptide comprises an antigen binding site that binds CD33. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

III. Therapeutic Compositions and their Use

The invention provides methods for treating cancer using a multi-specific binding protein described herein and/or a pharmaceutical composition described herein. The methods may be used to treat a variety of cancers which express CD33 by administering to a patient in need thereof a therapeutically effective amount of a multi-specific binding protein described herein.

The therapeutic method can be characterized according to the cancer to be treated. For example, in certain embodiments, the cancers are AML, myelodysplastic syndromes, chronic myelomonocytic leukemia, myeloid blast crisis of chronic myeloid leukemia, and ALLs.

For example, in certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to CD33: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

In embodiments of the present invention, the cancer to be treated is selected from acute myeloid leukemia (AML), myelodysplastic syndrome (MDS), acute lymphoblastic leukemia (ALL), myeloproliferative neoplasms (MPNs), lymphoma, non-Hodgkin lymphomas, and classical Hodgkin lymphoma.

In some embodiments of the present invention, the cancer to be treated is AML. In some embodiments of the present invention, the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN). In some embodiments of the present invention, the AML is characterized by expression of CLL-1 on the AML leukemia stem cells (LSCs). In some embodiments of the present invention, the LSCs in an AML subject further express a membrane marker selected from CD34, CD38, CD123, TIM3, CD25, CD32, and CD96. In some embodiments of the present invention, the AML is characterized as a minimal residual disease (MRD). In some embodiments of the present invention, the MRD of AML is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)).

In certain embodiments of the present invention, the cancer is MDS selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U).

It is contemplated that the multi-specific binding proteins and/or pharmaceutical compositions of the present disclosure can be used to treat a variety of cancers, not limited to cancers in which the cancer cells express CD33. For example, in certain embodiments, the multi-specific binding proteins and/or pharmaceutical compositions disclosed herein can be used to treat cancers that are associated with CD33-expressing immune cells. CD33 is expressed on many myeloid lineages, and tumor-infiltrating myeloid cells (e.g., tumor-associated macrophages) may contribute to cancer progression and metastasis. Therefore, the methods disclosed herein may be used to treat a variety of cancers in which CD33 is expressed, whether on cancer cells or on immune cells.

In certain embodiments, the multi-specific binding proteins and/or pharmaceutical compositions of the present disclosure can be used to treat cancers that express an Fc receptor with a higher binding affinity to Fc (e.g., IgG1 Fc) than CD16. In certain embodiments, the Fc receptor is FcγRI. In certain embodiments, the Fc receptor is expressed on cancer cells and/or other cells in the tumor microenvironment.

In certain embodiments, the patient has effector cells (e.g., NK cells) that express a CD16 variant with V158F substitution. In certain embodiments, the patient has a single nucleotide polymorphism (SNP) in the CD16 gene that causes V158F substitution. In certain embodiments, the patient has such an SNP in only one allele. In certain embodiments, the patient has such an SNP or SNPs in both alleles.

### IV. Combination Therapy

Another aspect of the invention provides for combination therapy. Multi-specific binding proteins described herein be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDCl₇ Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein, which contains a CD33-binding site described herein, and a pharmaceutically acceptable carrier.

In certain embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:3, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:4. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:7, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:8. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:11, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:12. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:13, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:14. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:15, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:16. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:17, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:18. In certain embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:19, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:20.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g. 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/ml of disodium phosphate dihydrate, 0.7 to 1.1 mg/mL of sodium dihydrogen phosphate dihydrate, and 6.0 to 6.4 mg/mL of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation include an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg-about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues.

Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following examples are merely illustrative and are not intended to limit the scope or content of the invention in any way.

### Example 1. Kinetics and Affinity of Binding to Different Variants of CD33

Kinetics and affinity of a series of Fab fragments of anti-CD33 antibodies with different CD33 variants (human CD33 ECD, cyno CD33 ECD, V-domain of human CD33, C-domain or human CD33 and selected CD33 SNPs) were assessed by surface plasmon resonance using Biacore 8K instrument (GE Healthcare). Anti-Fab antibody was immobilized on a CMS chip using standard amine coupling chemistry. CD33 FABs were captured on the anti-Fab chip at a density of ˜100 RU. Solutions containing different concentrations of soluble monomeric CD33 or its domains were injected over the captured FABs and control surfaces at 30 μl/min at 37° C. Surfaces were regenerated between cycles by quick injection of 10 mM glycine, pH 1.8. To obtain kinetic rate constants double-referenced data were fit to a 1:1 interaction model using Biacore 8K Evaluation software (GE Healthcare). The equilibrium binding constant K_{D} was determined by the ratio of binding rate constants k_{d}/kₐ.

### Octet Platform-Based Kinetic and Affinity Analyis

ForteBio affinity measurements were performed on an Octet HTX generally as described in Estep et al., High throughput solution-based measurement of antibody-antigen affinity and epitope binning. Mabs 5(2), 270-278 (2013). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model. The results are shown in Table 11.

TABLE 11
Kinetic parameters of human CD33 binding to IgG
antibodies measured by BLI.

| IgG ADI Name | K_{D} (M) | kₒₙ (1/M/s) | k_{off} (1/s) |
|---|---|---|---|
| ADI-10154 | 3.70E−09 | 2.46E+05 | 8.97E−04 |
| ADI-10155 | 5.30E−09 | 2.29E+05 | 1.21E−03 |
| ADI-10157 | 4.20E−09 | 7.99E+04 | 3.36E−04 |
| ADI-10158 | 2.60E−09 | 5.86E+05 | 1.55E−03 |
| ADI-10159 | 5.80E−10 | 3.48E+05 | 2.00E−04 |
| ADI-10160 | 5.10E−09 | 6.38E+05 | 3.25E−03 |
| ADI-10161 | 3.70E−09 | 5.87E+05 | 2.17E−03 |
| ADI-10163 | 6.80E−09 | 6.65E+05 | 4.53E−03 |
| ADI-10164 | 4.30E−09 | 3.44E+05 | 1.48E−03 |
| ADI-10165 | 4.00E−09 | 7.79E+05 | 3.11E−03 |
| ADI-10167 | 3.90E−09 | 1.00E+06 | 3.93E−03 |
| ADI-10168 | 1.00E−08 | 4.76E+05 | 4.78E−03 |
| ADI-10173 | 9.70E−09 | 3.24E+05 | 3.14E−03 |
| ADI-10177 | 3.60E−09 | 6.01E+05 | 2.14E−03 |
| ADI-11776 | 2.94E−10 | 6.79E+05 | 2.00E−04 |
| ADI-11801 | 4.57E−10 | 4.38E+05 | 2.00E−04 |
| ADI-11802 | 4.53E−10 | 4.41E+05 | 2.00E−04 |
| ADI-11807 | 3.07E−10 | 6.51E+05 | 2.00E−04 |
| ADI-11809 | 3.13E−10 | 6.38E+05 | 2.00E−04 |
| ADI-11812 | 1.91E−09 | 1.66E+05 | 3.15E−04 |
| ADI-11815 | 1.44E−09 | 1.59E+05 | 2.29E−04 |
| ADI-11819 | 2.43E−10 | 8.24E+05 | 2.00E−04 |
| ADI-11825 | 5.40E−10 | 6.96E+05 | 3.76E−04 |
| ADI-11826 | 3.93E−10 | 6.19E+05 | 2.43E−04 |
| ADI-11828 | 8.10E−10 | 6.58E+05 | 5.33E−04 |
| ADI-11830 | 1.20E−09 | 7.70E+05 | 9.23E−04 |
| ADI-11835 | 2.81E−10 | 8.44E+05 | 2.37E−04 |
| ADI-11839 | 2.45E−09 | 7.04E+05 | 1.72E−03 |
| Lintuzumab | 2.21E−09 | 4.31E+05 | 9.52E−04 |

### Example 2. CD33 Antibodies Bind to Human CD33 with High Affinity and Cross-React with Cyno CD33

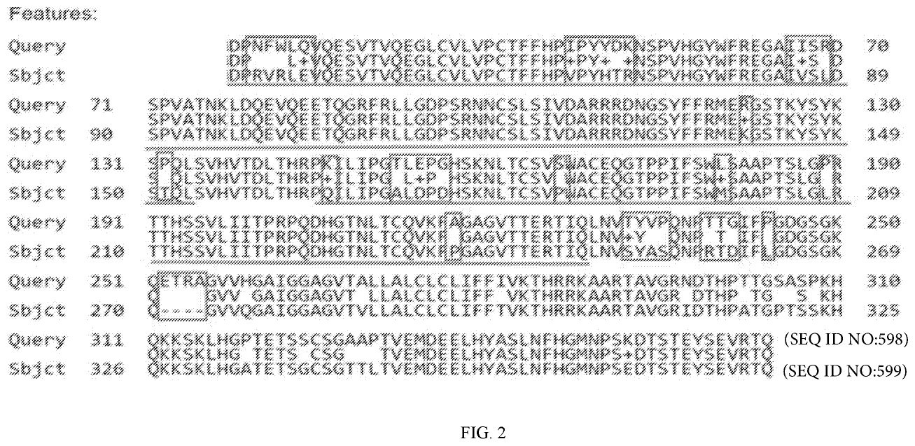

Despite rather high homology between human and cyno CD33 (87% in the ECD), most of commercially available anti-CD33 antibodies, e.g., lintuzumab, mylotarg, etc. lack cross-reactivity with cyno CD33. FIG. 2 shows alignment of full length human and cyno CD33 highlighting the differences in the primary sequence in the ECD domain.

Affinity of 29 Fab fragments binding to human and cyno CD33 ECD were assayed by Biacore analysis. Eight out of 29 antibodies show cross-reactivity with cyno CD33. Kinetic parameters of binding are given in Table 12. Data are compared to lintuzumab. Several antibodies show affinities >100 fold higher than lintuzumab.

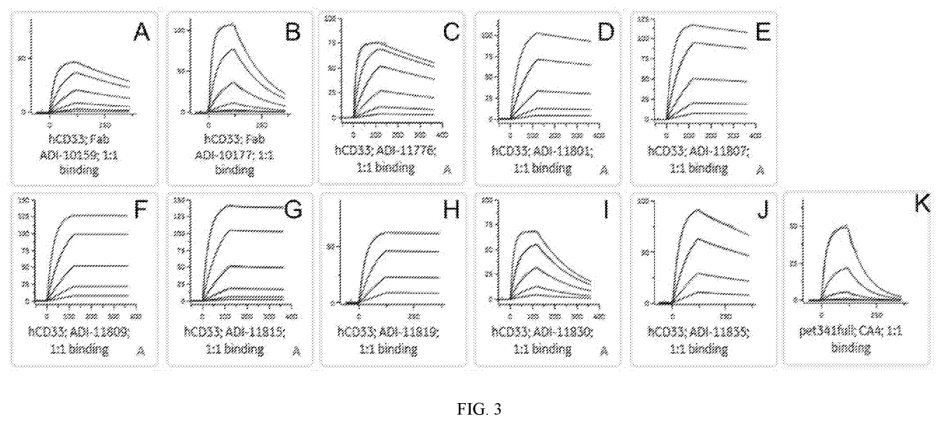

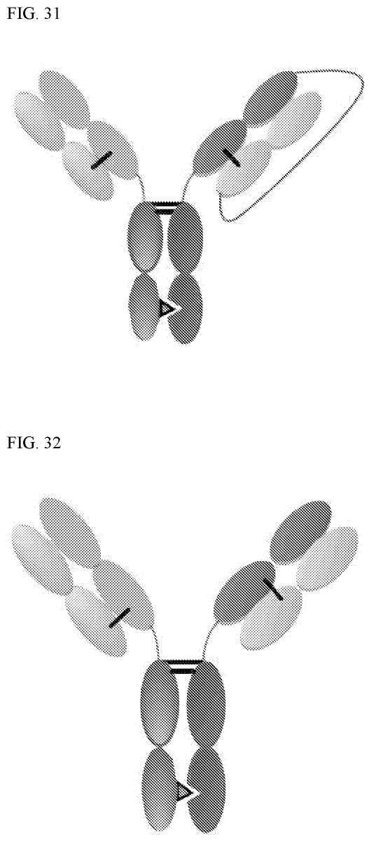

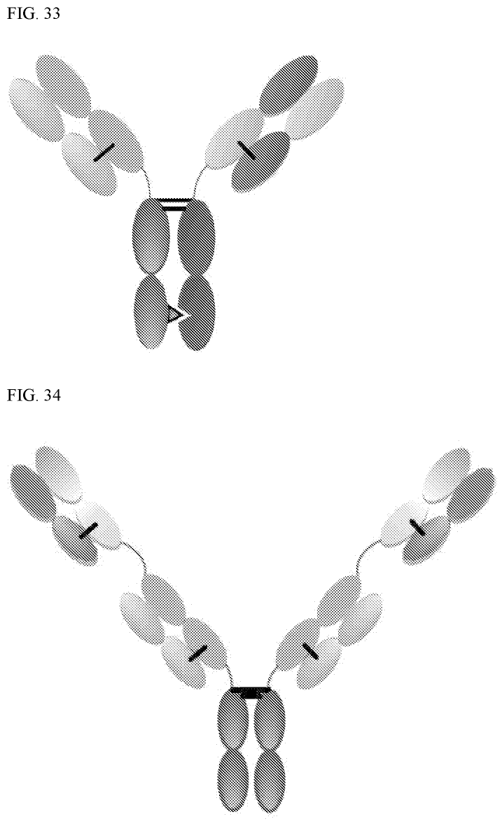

Binding of the Fab fragments from CD33 monoclonal antibodies to the human CD33 extracellular domain (ECD) was measured by Biacore at 37° C. The Biacore profile of ADI-10159 is shown in FIG. 3A; the Biacore profile of ADI-10177 is shown in FIG. 3B; the Biacore profile of ADI-11776 is shown in FIG. 3C; the Biacore profile of ADI-11801 in FIG. 3D; the Biacore profile of ADI-11807 is shown in FIG. 3E; the Biacore profile of ADI-11809 is shown in FIG. 3F; the Biacore profile of ADI-11815 is shown in FIG. 3G; the Biacore profile of ADI-11819 FIG. 3H; the Biacore profile of ADI-11830 is shown in FIG. 31; the Biacore profile of ADI-11835 is shown in FIG. 3J; and the Biacore profile of the Fab fragment from Lintuzumab is shown in FIG. FIG. 3K.

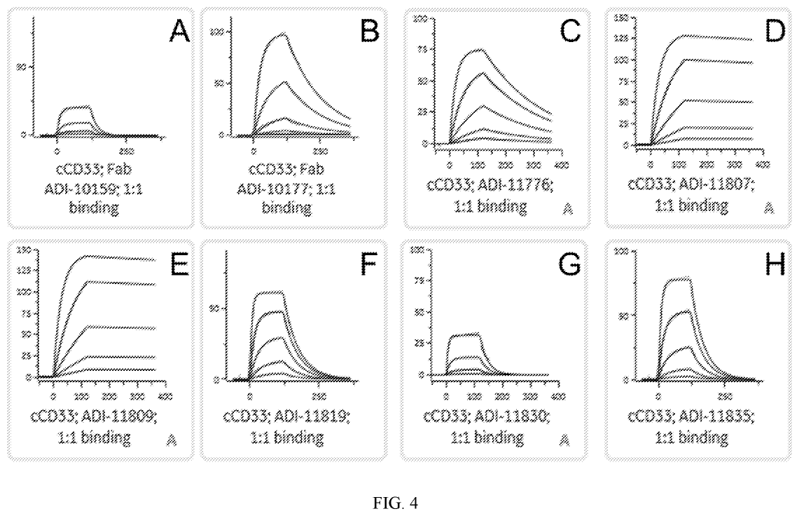

Binding of the Fab fragments from CD33 monoclonal antibodies to the cyno CD33 ECD was measured by Biacore at 37° C. The Biacore profile of ADI-10159 is shown in FIG. 4A; the Biacore profile of ADI-10177 in FIG. 4B; the Biacore profile of ADI-11776 is shown in FIG. 4C; the Biacore profile of ADI11807 is shown in FIG. 4D; the Biacore profile of ADI-11809 is shown in FIG. 4E; the Biacore profile of ADI-11819 is shown in FIG. 4F; the Biacore profile of ADI-11830 is shown in FIG. 4G; and the Biacore profile of ADI-11835 is shown in FIG. 4H.

Binding of the Fab fragments from CD33 monoclonal antibodies to V domain and C domain of human CD33 was measured by Biacore at 37° C. FIGS. 5A-5J represent binding to the V-domain; panels K-T represent binding to the C domain. Both FIGS. 5A and 5K are Biacore profiles of ADI-10159; both FIGS. 5B and 5L are Biacore profiles of ADI-10177; both FIGS. 5C and 5M are Biacore profiles of ADI-11776; both FIGS. 5D and 5N are Biacore profiles of ADI-11801; both FIGS. 5E and 5O are Biacore profiles of ADI-11807; both FIGS. 5F and 5P are Biacore profiles of ADI-11809; both FIGS. 5G and 5Q are Biacore profiles of ADI-11815; both FIGS. 5H and 5R are Biacore profiles of ADI-11819; both FIGS. 5I and 5S are Biacore profiles of ADI-11830; and FIGS. 5J and 5T are Biacore profiles of ADI-11835.

TABLE 12
Kinetic parameters of human CD33 ECD and cyno CD33 ECD
binding to Fabs measured by SPR at 37° C. No binding is
defined as absence of signal at highest concentration of 100 nM.

| | Human CD33 | | | Cyno CD33 | | |
|---|---|---|---|---|---|---|
| | kₐ | k_{d} | K_{D} | kₐ | k_{d} | K_{D} |
| Antibody | (1/Ms) | (1/s) | (nM) | (1/Ms) | (1/s) | (nM) |
| ADI-10159 | 4.29e5 | 1.95e−3 | 4.53 | 5.28e5 | 4.36e−2 | 74.9 |
| ADI-10177 | 5.58e5 | 6.36e−3 | 11.4 | 3.53e5 | 7.54e−3 | 21.4 |
| ADI-11776 | 1.6e6 | 1.24e−3 | 0.78 | 2.62e6 | 4.72e−3 | 1.81 |
| ADI-11807 | 1.14e6 | 3.32e−4 | 0.29 | 9.98e5 | 1.51e−4 | 0.15 |
| ADI-11809 | 1.02e6 | 1.23e−6 | 0.0012 | 1.06e6 | 1.26e−4 | 0.118 |
| ADI-11819 | 2.84e6 | 5.65e−5 | 0.02 | 3.82e6 | 1.90e−2 | 4.96 |
| ADI-11830 | 1.99e6 | 5.47e−3 | 2.75 | 1.34e6 | 3.64e−2 | 27.8 |
| ADI-11835 | 2.6e6 | 1.43e−3 | 0.53 | 2.76e6 | 1.89e−2 | 6.86 |
| ADI-11815 | 3.39e5 | 7.91e−5 | 0.23 | No binding | | |
| ADI-11801 | 7.56e5 | 4.17e−4 | 0.55 | No binding | | |
| ADI-10152 | 2.46e5 | 9.14e−3 | 37.2 | No binding | | |
| ADI-10154 | 1.92e5 | 8.33e−3 | 43.4 | No binding | | |
| ADI-10155 | 1.70e5 | 6.85e−3 | 40.2 | No binding | | |
| ADI-10157 | 2.82e6 | 7.60e−3 | 2.69 | No binding | | |
| ADI-10158 | 4.75e5 | 1.49e−2 | 3.14 | No binding | | |
| ADI-10160 | 6.37e5 | 2.69e−2 | 42.2 | No binding | | |
| ADI-10161 | 5.74e5 | 1.18e−2 | 20.6 | No binding | | |
| ADI-10163 | 5.15e5 | 2.75e−2 | 53.5 | No binding | | |
| ADI-10164 | 3.19e5 | 1.01e−2 | 31.5 | No binding | | |
| ADI-10165 | 5.91e5 | 1.88e−2 | 31.8 | No binding | | |
| ADI-10167 | 1.25e6 | 3.07e−2 | 24.4 | No binding | | |
| ADI-10168 | 3.75e5 | 1.75e−2 | 46.6 | No binding | | |
| ADI-10173 | 2.12e5 | 1.25e−2 | 58.8 | No binding | | |
| ADI-11802 | 4.9e5 | 1.54e−3 | 3.14 | No binding | | |
| ADI-11812 | 3.96e5 | 4.86e−4 | 1.23 | No binding | | |
| ADI-11825 | 2.13e6 | 3.8e−3 | 1.78 | No binding | | |
| ADI-11826 | 1.76e6 | 4.63e−3 | 2.63 | No binding | | |
| ADI-11828 | 1.32e6 | 3.51e−3 | 2.67 | 1.03e6 | 5.48e−2 | 53.5 |

| | | | | |
|---|---|---|---|---|
| ADI-11839 | 8.74e5 | 1.62e−2 | 18.6 | No binding |
| Lintuzumab | 7.35e5 | 1.22e−2 | 16.7 | No binding |

### Mapping of Binding Interface to Individual Domains of CD33

The binding interface between the Fab fragment of each CD33 antibody to CD33 was mapped. FIGS. 5A-5T show binding of Fab fragments of different CD33 antibodies to individual domains of human CD33 (V domain and C domain). No binding to C domain was observed for any antibody tested. ADI-11815 did not bind to either V or C domain, suggesting that it requires a unique conformational epitope.

Table 13 shows a comparison between kinetics of binding to full ECD of human CD33 and a V domain. ADI-10159, ADI-11176, ADI-11807, ADI-11830, ADI-11835, ADI-11801, ADI-10155, ADI-11802, ADI-11825, ADI-11826, ADI-11828, and ADI-11839 show similar kinetics suggesting that the epitope for these antibodies are located entirely in the V domain. Reduced binding to V domain is observed for ADI-10177, ADI-11809 ADI-11819, ADI-10157, ADI-10158, and ADI-10164, suggesting that these antibodies bind to a conformational epitope, partially located in the V domain.

Kinetics of binding to the C domain of human CD33 was also measured with ADI-10152, ADI-10154, ADI-10155, ADI-10157, ADI-10158, ADI-10160, ADI-10161, ADI-10163, ADI-10164, ADI-10165, ADI-10167, ADI-10168, ADI-10173, ADI-11802, ADI-11812, ADI-11825, ADI-11826, ADI-11828, and ADI-11839. None of these antibodies bound the C domain of human CD33.

TABLE 13
Biacore analysis of FABs binding to recombinant full-
length ECD and V domain of human CD33 performed
at 37° C. Asterisk indicates antibodies that bind to
a conformational epitope partially located in V domain.

| | Human CD33 V domain | | | Human CD33 | | |
|---|---|---|---|---|---|---|
| | kₐ | k_{d} | K_{D} | kₐ | k_{d} | K_{D} |
| Antibody | (1/Ms) | (1/s) | (nM) | (1/Ms) | (1/s) | (nM) |
| ADI-10159 | 9.05e5 | 5.0e−3 | 5.56 | 4.29e5 | 1.95e−3 | 4.53 |
| ADI-10177* | 1.56e6 | 8.57e−2 | 54.8 | 5.58e5 | 6.36e−3 | 11.4 |
| ADI-11776 | 4.52e6 | 1.43e−2 | 0.33 | 1.6e6 | 1.24e−3 | 0.78 |
| ADI-11807 | 3.07e6 | 8.47e−4 | 0.28 | 1.14e6 | 3.32e−4 | 0.29 |
| ADI-11809* | 2.6e6 | 7.69e−4 | 0.3 | 1.02e6 | 1.23e−6 | 0.0012 |
| ADI-11819 | 6.75e6 | 9.16e−4 | 0.14 | 2.84e6 | 5.65e−5 | 0.02 |
| ADI-11830 | 6.67e6 | 2.99e−2 | 4.5 | 1.99e6 | 5.47e−3 | 2.75 |
| ADI-11835 | 7.6e6 | 8.68e−3 | 1.14 | 2.6e6 | 1.43e−3 | 0.53 |
| ADI-11815 | No binding | | | 3.39e5 | 7.91e−5 | 0.23 |
| ADI-11801 | 1.8e6 | 1.25e−3 | 0.69 | 7.56e5 | 4.17e−4 | 0.55 |
| ADI-10152 | 4.88e5 | 4.09e−2 | 100 | 2.46e5 | 9.14e−3 | 37.2 |
| ADI-10154 | 1.52e6 | 3.35e−1 | 220 | 1.92e5 | 8.33e−3 | 43.4 |
| ADI-10155 | 5.48e5 | 4.67e−2 | 85.2 | 1.70e5 | 6.85e−3 | 40.2 |
| ADI-10157* | 1.12e5 | 1.51e−2 | 134 | 2.82e6 | 7.60e−3 | 2.69 |
| ADI-10158* | 1.04e6 | 1.33e−1 | 127 | 4.75e5 | 1.49e−2 | 3.14 |
| ADI-10160 | 1.59e6 | 3.27e−1 | 206 | 6.37e5 | 2.69e−2 | 42.2 |
| ADI-10161 | 1.29e6 | 1.24e−1 | 95.8 | 5.74e5 | 1.18e−2 | 20.6 |
| ADI-10163 | No binding | | | 5.15e5 | 2.75e−2 | 53.5 |
| ADI-10164 | 1.29e6 | 3.7e−1 | 286 | 3.19e5 | 1.01e−2 | 31.5 |
| ADI-10165 | No binding | | | 5.91e5 | 1.88e−2 | 31.8 |
| ADI-10167 | No binding | | | 1.25e6 | 3.07e−2 | 24.4 |
| ADI-10168 | No binding | | | 3.75e5 | 1.75e−2 | 46.6 |
| ADI-10173 | No binding | | | 2.12e5 | 1.25e−2 | 58.8 |
| ADI-11802 | 1.34e6 | 4.35e−3 | 3.2 | 4.9e5 | 1.54e−3 | 3.14 |
| ADI-11812 | No binding | | | 3.96e5 | 4.86e−4 | 1.23 |
| ADI-11825 | 4.93e6 | 1.95e−2 | 3.95 | 2.13e6 | 3.8e−3 | 1.78 |
| ADI-11826 | 5.12e6 | 2.25e−3 | 4.38 | 1.76e6 | 4.63e−3 | 2.63 |
| ADI-11828 | 3.37e6 | 2.13e−2 | 6.3 | 1.32e6 | 3.51e−3 | 2.67 |
| ADI-11839 | 9.19e6 | 4.1e−1 | 44.6 | 8.74e5 | 1.62e−2 | 18.6 |

### Antibodies Recognize CD33 Independent of its Glycosylation Status.

The ability of anti-CD33 antibodies to recognize glycosylated CD33 was assayed. Table 14 shows that antibodies recognize V domain independent of its glycosylation status. Human CD33 is heavily glycosylated with 2 glycosylation sites located in the V domain. Differences in the glycosylation level of CD33 in different cells have been reported in the literature. Glycosylation can potentially disturb antibody binding to the target. In some samples, the V domain was deglycosylated by PNGase before testing. De-glycosylation status was confirmed by a shift on SDS-PAGE and MS. All antibodies tested in Table 14 bound to deglycosylated V CD33 similarly to the fully glycosylated version except for ADI-10163, ADI-10165, ADI-10167, and ADI-10173.

TABLE 14
Biacore analysis of FABs binding to fully glycosylated
vs deglycosylated V domain performed at 37° C.

| | Deglycosylated V domain | | | V domain | | |
|---|---|---|---|---|---|---|
| Antibody | kₐ (1/Ms) | k_{d} (1/s) | K_{D} (nM) | kₐ (1/Ms) | k_{d} (1/s) | K_{D} (nM) |
| ADI-10159 | 9.78e5 | 1.2e−2 | 12.3 | 9.05e5 | 5.0e−3 | 5.56 |
| ADI-10177 | 1.88e6 | 7.01e−2 | 38.2 | 1.56e6 | 8.57e−2 | 54.8 |
| ADI-11776 | 4.85e6 | 1.36e−3 | 0.28 | 4.52e6 | 1.43e−2 | 0.33 |
| ADI-11807 | 3.9e6 | 7.45e−4 | 0.19 | 3.07e6 | 8.47e−4 | 0.28 |
| ADI-11809 | 3.4e6 | 7.11e−4 | 0.21 | 2.6e6 | 7.69e−4 | 0.3 |
| ADI-11819 | 9.15e6 | 1.03e−3 | 0.11 | 6.75e6 | 9.16e−4 | 0.14 |
| ADI-11830 | 6.8e6 | 2.69e−2 | 3.95 | 6.67e6 | 2.99e−2 | 4.5 |
| ADI-11835 | 7.94e6 | 7.79e−3 | 0.98 | 7.6e6 | 8.68e−3 | 1.14 |
| ADI-11801 | 2.1e6 | 1.15e−3 | 0.55 | 1.8e6 | 1.25e−3 | 0.69 |
| ADI-10152 | 1.05e5 | 7.7e−2 | 76.8 | 4.88e5 | 4.09e−2 | 100 |
| ADI-10154 | 3.13e5 | 6.55e−2 | 209 | 1.52e6 | 3.35e−1 | 220 |
| ADI-10155 | 1.25e6 | 1.32e−1 | 105 | 5.48e5 | 4.67e−2 | 85.2 |
| ADI-10157 | 4.11e5 | 2.61e−2 | 63.8 | 1.12e5 | 1.51e−2 | 134 |
| ADI-10158 | 2.63e6 | 2.31e−1 | 88.1 | 1.04e6 | 1.33e−1 | 127 |
| ADI-10160 | 1.7e6 | 2.25e−1 | 133 | 1.59e6 | 3.27e−1 | 206 |
| ADI-10161 | 2.29e6 | 1.96e−1 | 85.6 | 1.29e6 | 1.24e−1 | 95.8 |
| ADI-10163 | 1.68e6 | 2.56e−1 | 152 | No binding | | |
| ADI-10164 | 8.11e5 | 1.16e−1 | 143 | 1.29e6 | 3.7e−1 | 286 |
| ADI-10165 | 1.3e6 | 2.03e−1 | 156 | No binding | | |
| ADI-10167 | 1.66e6 | 1.28e−1 | 77.8 | No binding | | |
| ADI-10168 | No binding | | | No binding | | |
| ADI-10173 | 6.74e5 | 1.31e−1 | 200 | No binding | | |
| ADI-11802 | 1.57e6 | 4.17e−3 | 2.65 | 1.34e6 | 4.35e−3 | 3.2 |
| ADI-11812 | No binding | | | No binding | | |
| ADI-11825 | 5.78e6 | 1.8e−2 | 3.11 | 4.93e6 | 1.95e−2 | 3.95 |
| ADI-11826 | 5.37e6 | 1.98e−2 | 3.69 | 5.12e6 | 2.25e−3 | 4.38 |
| ADI-11828 | 3.588e6 | 1.89e−2 | 4.87 | 3.37e6 | 2.13e−2 | 6.3 |
| ADI-11839 | 1.65e6 | 1.16e−1 | 70.3 | 9.19e6 | 4.1e−1 | 44.6 |

### CD33 Antibodies Bind to the R69G SNP of CD33.

The ability of anti-CD33 antibodies to recognize the R69G mutation in CD33 was assayed. Although several SNPs have been described for CD33, R69G is particularly prominent, occurring in 39-42% of the population. Table 15 shows antibodies binding to human CD33 containing the R69G mutation.

TABLE 15
Biacore analysis of FABs binding to CD33 R69G.

| | Human CD33 R69G | | | Human CD33 | | |
|---|---|---|---|---|---|---|
| Antibody | kₐ (1/Ms) | k_{d} (1/s) | K_{D} (nM) | kₐ (1/Ms) | k_{d} (1/s) | K_{D} (nM) |
| ADI-10159 | 4.26e5 | 2.43e−3 | 5.68 | 4.29e5 | 1.95e−3 | 4.53 |
| ADI-10177 | 2.3e5 | 2.01e−3 | 87.4 | 5.58e5 | 6.36e−3 | 11.4 |
| ADI-11776 | 1.98e6 | 4.26e−4 | 0.22 | 1.6e6 | 1.24e−3 | 0.78 |
| ADI-11807 | 5.97e5 | 2.59e−4 | 0.43 | 1.14e6 | 3.32e−4 | 0.29 |
| ADI-11809 | 8.15e5 | 1.79e−4 | 0.22 | 1.02e6 | 1.23e−6 | 0.0012 |
| ADI-11819 | 2.78e6 | 2.44e−4 | 0.09 | 2.84e6 | 5.65e−5 | 0.02 |
| ADI-11830 | 2.27e6 | 6.63e−3 | 2.94 | 1.99e6 | 5.47e−3 | 2.75 |
| ADI-11835 | 3.07e6 | 2.05e−3 | 0.67 | 2.6e6 | 1.43e−3 | 0.53 |
| ADI-11815 | 3.00e5 | 1.3e−3 | 4.34 | 3.39e5 | 7.91e−5 | 0.23 |
| ADI-11801 | No binding | | | 7.56e5 | 4.17e−4 | 0.55 |
| ADI-10152 | 2.88e5 | 2.18e−2 | 75.8 | 2.46e5 | 9.14e−3 | 37.2 |
| ADI-10154 | 1.53e5 | 2.83e−2 | 186 | 1.92e5 | 8.33e−3 | 43.4 |
| ADI-10155 | 2.33e5 | 1.05e−2 | 45.0 | 1.70e5 | 6.85e−3 | 40.2 |
| ADI-10157 | 1.56e6 | 2.87e−2 | 17.8 | 2.82e6 | 7.60e−3 | 2.69 |
| ADI-10158 | 5.30e5 | 3.31e−2 | 62.3 | 4.75e5 | 1.49e−2 | 3.14 |
| ADI-10160 | 8.36e5 | 5.33e−2 | 63.7 | 6.37e5 | 2.69e−2 | 42.2 |
| ADI-10161 | 6.29e5 | 3.07e−2 | 48.9 | 5.74e5 | 1.18e−2 | 20.6 |
| ADI-10163 | 5.59e5 | 5.9e−2 | 106 | 5.15e5 | 2.75e−2 | 53.5 |
| ADI-10164 | 3.08e5 | 3.5e−2 | 113 | 3.19e5 | 1.01e−2 | 31.5 |
| ADI-10165 | 6.9e5 | 5.02e−2 | 72.7 | 5.91e5 | 1.88e−2 | 31.8 |
| ADI-10167 | 1.22e6 | 8.74e−2 | 71.6 | 1.25e6 | 3.07e−2 | 24.4 |
| ADI-10168 | 4.85e5 | 3.07e−2 | 63.5 | 3.75e5 | 1.75e−2 | 46.6 |
| ADI-10173 | No binding | | | 2.12e5 | 1.25e−2 | 58.8 |
| ADI-11802 | No binding | | | 4.9e5 | 1.54e−3 | 3.14 |
| ADI-11812 | 3.38e5 | 2.28e−3 | 6.74 | 3.96e5 | 4.86e−4 | 1.23 |
| ADI-11825 | 1.86e6 | 6.95e−3 | 3.74 | 2.13e6 | 3.8e−3 | 1.78 |
| ADI-11826 | 1.89e6 | 7.23e−3 | 3.83 | 1.76e6 | 4.63e−3 | 2.63 |
| ADI-11828 | 1.16e6 | 7.00e−3 | 6.03 | 1.32e6 | 3.51e−3 | 2.67 |

| | | | | |
|---|---|---|---|---|
| ADI-11839 | No binding | 8.74e5 | 1.62e−2 | 18.6 |

### CD33 Antibodies Bind to the S128N SNP of CD33

The ability of anti-CD33 antibodies to recognize the S128N mutation in CD33 was assayed. Table 16 shows antibodies binding to human CD33 containing the S128N mutation. The binding affinity of ADI-10152, ADI-10154, ADI-10157, ADI-10158, ADI-10163, ADI-10164, ADI-10165, ADI-10167, ADI-10168, and ADI-10173 to human CD33 was impaired by the S128N SNP.

TABLE 16
Biacore analysis of FABs binding to CD33 S128N.

| | Human CD33 S128N | | | Human CD33 | | |
|---|---|---|---|---|---|---|
| | kₐ | k_{d} | K_{D} | kₐ | k_{d} | K_{D} |
| Antibody | (1/Ms) | (1/s) | (nM) | (1/Ms) | (1/s) | (nM) |
| ADI-10152 | 2.74e5 | 3.39e−2 | 124 | 2.46e5 | 9.14e−3 | 37.2 |
| ADI-10154 | 1.93e5 | 5.39e−2 | 279 | 1.92e5 | 8.33e−3 | 43.4 |
| ADI-10155 | 1.87e5 | 1.59e−2 | 85 | 1.70e5 | 6.85e−3 | 40.2 |
| ADI-10157 | 5.65e4 | 9.24e−3 | 163 | 2.82e6 | 7.60e−3 | 2.69 |
| ADI-10158 | 5.73e5 | 4.17e−2 | 72.8 | 4.75e5 | 1.49e−2 | 3.14 |
| ADI-10160 | 7.86e5 | 6.73e−2 | 85.7 | 6.37e5 | 2.69e−2 | 42.2 |
| ADI-10161 | 6.05e5 | 3.85e−2 | 63.6 | 5.74e5 | 1.18e−2 | 20.6 |
| ADI-10163 | 5.07e5 | 7.48e−2 | 147 | 5.15e5 | 2.75e−2 | 53.5 |
| ADI-10164 | 1.93e5 | 5.39e−2 | 279 | 3.19e5 | 1.01e−2 | 31.5 |
| ADI-10165 | 6.41e5 | 5.82e−2 | 90.7 | 5.91e5 | 1.88e−2 | 31.8 |
| ADI-10167 | 1.28e6 | 1.27e−1 | 98.6 | 1.25e6 | 3.07e−2 | 24.4 |
| ADI-10168 | 4.04e5 | 4.69e−2 | 116 | 3.75e5 | 1.75e−2 | 46.6 |
| ADI-10173 | 1.83e5 | 3.42e−2 | 187 | 2.12e5 | 1.25e−2 | 58.8 |
| ADI-11802 | 6.62e5 | 5.90e−4 | 0.809 | 4.9e5 | 1.54e−3 | 3.14 |
| ADI-11812 | 5.56e5 | 1.12e−3 | 2.01 | 3.96e5 | 4.86e−4 | 1.23 |
| ADI-11825 | 2.10e6 | 6.49e−3 | 3.09 | 2.13e6 | 3.8e−3 | 1.78 |
| ADI-11826 | 2.17e6 | 5.74e−3 | 2.65 | 1.76e6 | 4.63e−3 | 2.63 |
| ADI-11828 | 1.37e6 | 7.42e−3 | 5.41 | 1.32e6 | 3.51e−3 | 2.67 |
| ADI-11839 | 9.63e5 | 2.84e−2 | 29.5 | 8.74e5 | 1.62e−2 | 18.6 |

### ADI-11815 Recognizes a Unique Conformational Epitope.

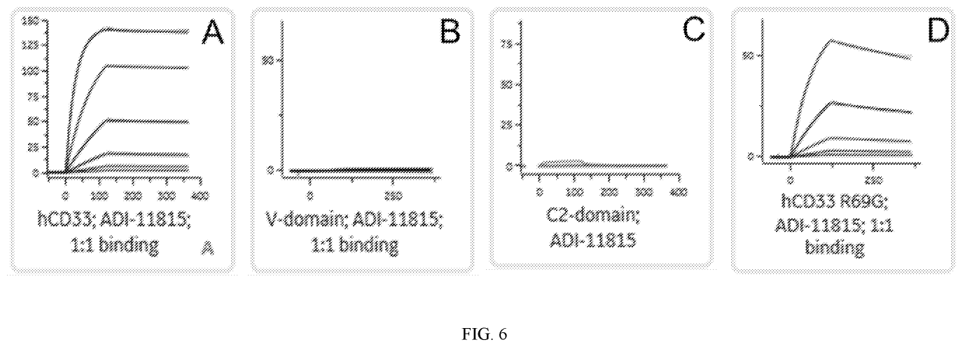

The binding epitope of the CD33-binding domain of ADI-11815 was assayed. FIG. 6 and Table 17 demonstrate that ADI-11815 has a unique conformational epitope. This antibody binds to the full-length ECD of human CD33, but not to individual domains and does not cross-block with lintuzumab.

TABLE 17
Kinetic parameters of ADI-11815 Fab binding to different domains and SNP R69G of human CD33.

| | | | | | | | | | | | | | Cross- |
|---|---|---|---|---|---|---|---|---|---|---|---|---|---|
| | hCD33 | | | V domain | | | C domain | | | hCD33 R69G SNP | | | blocking |
| | kₐ | k_{d} | K_{D} | kₐ | k_{d} | K_{D} | kₐ | k_{d} | K_{D} | kₐ | k_{d} | K_{D} | with |
| | (1/Ms) | (1/s) | (nM) | (1/Ms) | (1/s) | (nM) | (1/Ms) | (1/s) | (nM) | (1/Ms) | (1/s) | (nM) | lintuzumab |
| ADI- | 3.39e5 | 7.91e−5 | 0.23 | No binding | | | No binding | | | 3.00eS | 1.3e−3 | 4.34 | No |
| 11815 | | | | | | | | | | | | | |

ADI-11801 Binds to a Unique Epitope that Includes R69.

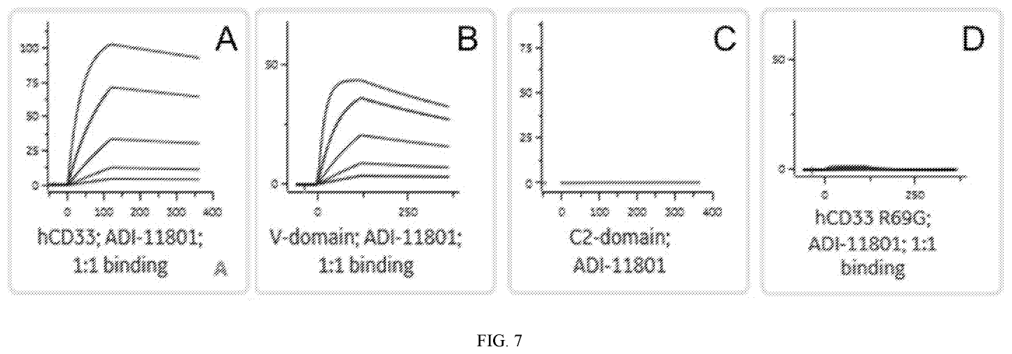

The binding epitope of CD33-binding domain including ADI-11815 was assayed. FIG. 7 and Table 18 demonstrate the epitope on CD33 that ADI-11801 recognize. Its binding to the human CD33 ECD is abrogated by R69G mutation.

TABLE 18
Kinetic parameters of ADI-11801 Fab binding to different domains of CD33 and SNP R69G.

| | | | | | | | | | | | | | Cross- |
|---|---|---|---|---|---|---|---|---|---|---|---|---|---|
| | hCD33 | | | V domain | | | C domain | | | hCD33 R69G SNP | | | blocking |
| | kₐ | k_{d} | K_{D} | kₐ | k_{d} | K_{D} | kₐ | k_{d} | K_{D} | kₐ | k_{d} | K_{D} | with |
| | (1/Ms) | (1/s) | (nM) | (1/Ms) | (1/s) | (nM) | (1/Ms) | (1/s) | (nM) | (1/Ms) | (1/s) | (nM) | lintuzumab |
| ADI- | 7.56e5 | 4.17e−4 | 0.55 | 1.80e6 | 1.25e−3 | 0.69 | No binding | | | No binding | | | No |
| 11801 | | | | | | | | | | | | | |

### Example 3: Assessment of TriNKET Binding to Cell Expressing Human NKG2D

The ability of TriNKETs that include an NKG2D-binding domain and a CD33-binding domain to bind to NKG2D was assayed. EL4 cells transduced with human NKG2D and human KHYG-1 cells were used to test binding to cell-expressed human NKG2D. TriNKETs were diluted to the top concentration, and then diluted serially. The mAb or TriNKET dilutions were used to stain cells, and binding of the TriNKET or mAb was detected using a fluorophore-conjugated anti-human IgG secondary antibody. Cells were analyzed by flow cytometry, binding MFI was normalized to secondary antibody controls to obtain fold over background values.

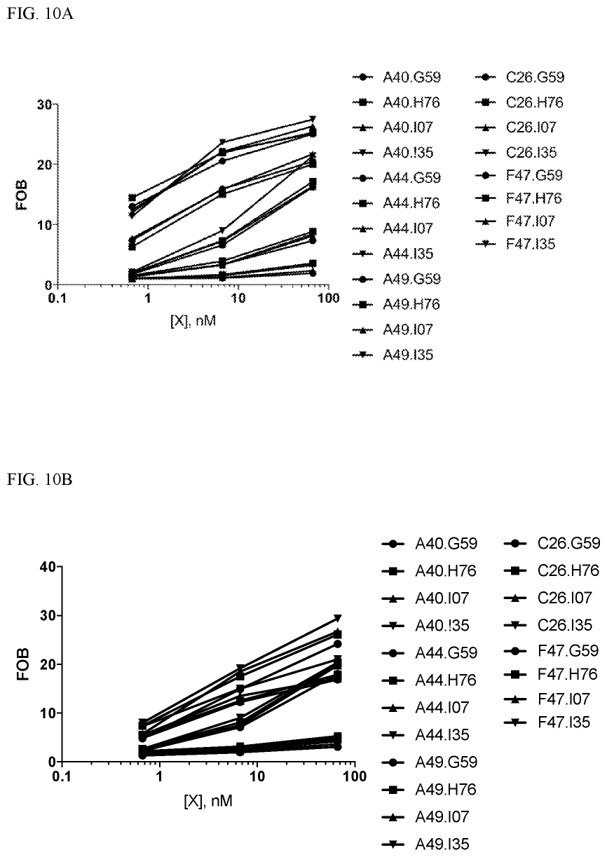

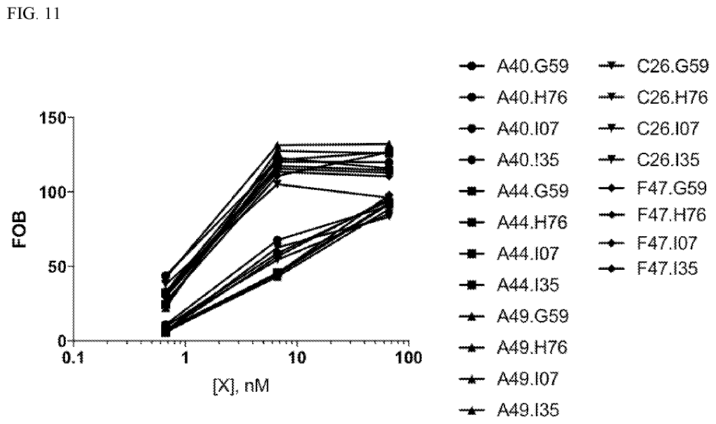

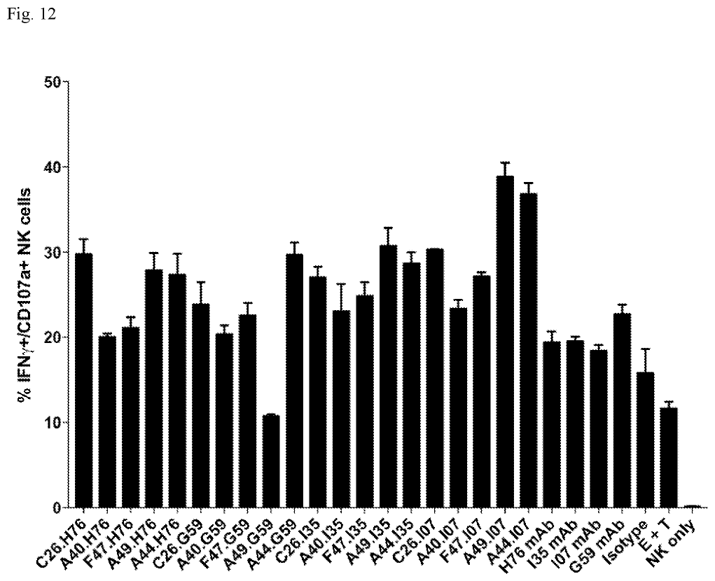

FIGS. 10A-10B show binding of CD33-targeting TriNKETs to human NKG2D expressed on EL4 (top) or KHYG-1 (bottom) cells. FOB binding signal was similar on both EL4-hNKG2D cells and KHYG-1 cells, the ranking of binding between clones was also maintain on the two cell lines. FIG. 10A shows binding of CD33-targeting TriNKETs to human NKG2D expressed on EL4 cells. FIG. 10B shows binding of CD33-targeting TriNKETs to human NKG2D expressed on KHYG-1 cells. FOB binding signal was similar on both EL4-hNKG2D cells and KHYG-1 cells, the ranking of binding between clones was also maintain on the two cell lines.

### Example 4: Assessment of TriNKET or mAb Binding to Cell Expressed Human Cancer Antigens

The ability of TriNKETs that include an NKG2D-binding domain and a CD33-binding domain to bind to CD33 was assayed. The Molm-13, human AML cell line, was used to assess binding of monoclonal antibodies to CD33 expressed on the cell surface. mAbs were diluted to 2 μg/mL, and mAb dilutions were used to stain cells. Bound antibody was detected using a fluorophore conjugated anti-human IgG secondary antibody. Cells were analyzed by flow cytometry, binding to cell-expressed CD33 was compared to isotype stained and unstained cell populations.

Human cancer cell lines expressing CD33 were used to assess tumor antigen binding of TriNKETs derived from different NKG2D-targeting clones. The human AML cell line Molm-13 was used to assess binding of TriNKETs to cell expressed CD33. TriNKETs were diluted, and were incubated with the respective cells. Binding of the TriNKET was detected using a fluorophore conjugated anti-human IgG secondary antibody. Cells were analyzed by flow cytometry, binding MFI to cell expressed CD33 was normalized to secondary antibody controls to obtain fold over background values.

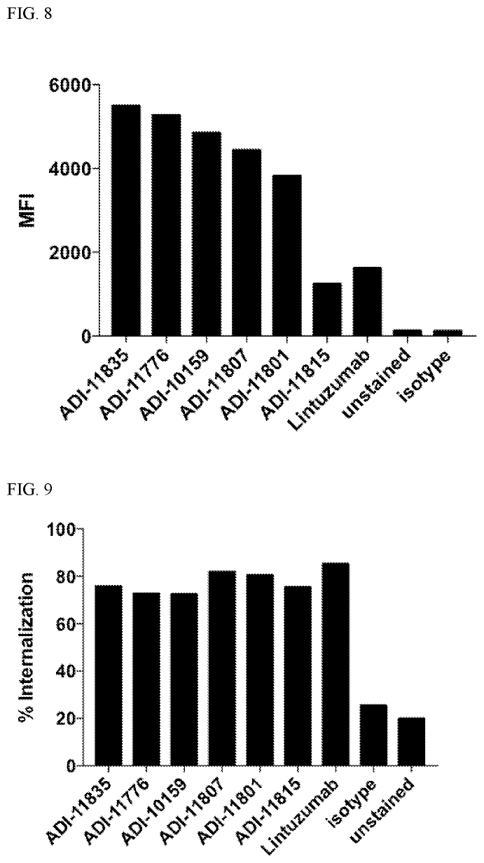

The ability of CD33 TriNKETs to bind CD33 expressed on Molm-13 cells was tested. FIG. 8 shows binding of TriNKETs targeting CD33 to Molm-shows binding MFI of six anti-CD33 antibodies to CD33 expressed on Molm-13 cells. All six antibodies bound to cell-expressed CD33. Five of the six antibodies show higher MFI binding signal compared to Lintuzumab.

The ability of CD33 TriNKETs to induce rested NK cell-mediated killing of Molm-13 AML cells was tested. Four different CD33-targeting domains were used with five NKG2D-targeting domains to make a total of 20 different TriNKETs. Regardless of the NKG2D-targeting domain used in the TriNKET, binding to CD33 was conserved for a single CD33-targeting domain.

### Example 5: Assessment of TriNKET or mAb Internalization

Internalization of TriNKETs after binding to CD33 on cell surface was assayed. The Molm-13, human AML cell line, was used to assess internalization of monoclonal antibodies bound to CD33 expressed on the cell surface. Monoclonal antibodies were diluted to 2 μg/mL, and mAb dilutions were used to stain cells. Following surface staining of CD33 samples were split, half the sample was placed at 37° C. overnight to facilitate internalization, with the other half of the sample bound antibody was detected using a fluorophore conjugated anti-human IgG secondary antibody. Cells were fixed after staining with the secondary antibody, and were stored at 4° C. overnight for analysis on the following day. After 24 hours at 37° C. samples were removed from the incubator, and bound antibody on the surface of the cells was detected using a fluorophore conjugated anti-human IgG secondary antibody. Samples were fixed and all samples were analyzed on the same day. Internalization of antibodies was calculated as follows: % internalization=(1−(sample MFI 24 hours/baseline MFI))*100%.

FIG. 9 shows internalization of anti-CD33 antibodies bound to the surface of Molm-13 cells after 24 hours. All the anti-CD33 antibodies showed similar internalization after 24 hours. Lintuzumab showed slightly higher internalization compare to other anti-CD33 antibodies.

### Example 6: Activation of Primary NK Cells by TriNKETs

The ability of TriNKETs that include an NKG2D-binding domain and a CD33-binding domain to activate primary NK cells was assayed. PBMCs were isolated from human peripheral blood buffy coats using density gradient centrifugation. Isolated PBMCs were washed and prepared for NK cell isolation. NK cells were isolated using a negative selection technique with magnetic beads, purity of isolated NK cells was typically >90% CD3-CD56+. Isolated NK cells were cultured in media containing 100 ng/mL IL-2 for activation or rested overnight without cytokine. IL-2-activated NK cells were used either 24-48 hours later; rested NK cells were always used the day after purification.

Human cancer cell lines expressing a cancer target of interest were harvested from culture, and cells were adjusted to 2×10⁶/mL. Monoclonal antibodies or TriNKETs targeting the cancer target of interest were diluted in culture media. Rested and/or activated NK cells were harvested from culture, cells were washed, and were resuspended at 2×10⁶/mL in culture media. IL-2, and fluorophore-conjugated anti-CD107a was added to the NK cells for the activation culture. Brefeldin-A and monensin were diluted into culture media to block protein transport out of the cell for intracellular cytokine staining. Into a 96-well plate 50 μl of tumor targets, mAbs/TriNKETs, BFA/monensin, and NK cells were added for a total culture volume of 200 μl. Plate was cultured for 4 hours before samples were prepared for FACS analysis.

Following the 4 hour activation culture, cells were prepared for analysis by flow cytometry using fluorophore-conjugated antibodies against CD3, CD56 and IFNγ. CD107a and IFNγ staining was analyzed in CD3-CD56+ populations to assess NK cell activation.

FIG. 12 shows TriNKET-mediated activation of rested human NK cells in co-culture with CD33-expressing THP-1 AML cells. Human NK cell activation was assessed using IFNγ production and CD107a degranulation as markers for activation. All TriNKETs and monoclonal antibodies activated human NK cells above the isotype control. Similar activity was observed for four different anti-CD33 antibodies. TriNKET activity was dependent upon the NKG2D-targeting clone, some clones provided better TriNKET mediated activation than others. NKG2D clones provided similar activity with each of the anti-CD33 targeting domains.

### Example 7: Primary Human NK Cell Cytotoxicity Assay

The ability of TriNKETs that include an NKG2D-binding domain and a CD33-binding domain to induce cytotoxicity of NK cells against CD33-expressing cells was assayed. PBMCs were isolated from human peripheral blood buffy coats using density gradient centrifugation. Isolated PBMCs were washed and prepared for NK cell isolation. NK cells were isolated using a negative selection technique with magnetic beads, purity of isolated NK cells was typically >90% CD3-CD56+. Isolated NK cells were cultured in media containing 100 ng/mL IL-2 or were rested overnight without cytokine. IL-2-activated or rested NK cells were used the following day in cytotoxicity assays.

KHYG-1 cells were maintained in 10% HI—FBS-RPMI-1640 with 10 ng/mL IL-2. The day before use as effector cells in killing assays KHYG-1 cells were harvest from culture, and cells were washed out of the IL-2-containing media. After washing KHYG-1 cells were resuspended in 10% HI—FBS-RPMI-1640, and were rested overnight without cytokine.

### DELFIA Cytotoxicity Assay:

Human cancer cell lines expressing a target of interest were harvested from culture, cells were washed with HBS, and were resuspended in growth media at 10⁶/mL for labeling with BATDA reagent (Perkin Elmer AD0116). Manufacturer instructions were followed for labeling of the target cells. After labeling cells were washed 3× with HBS, and were resuspended at 0.5-1.0×10⁵/mL in culture media. To prepare the background wells an aliquot of the labeled cells was put aside, and the cells were spun out of the media. 100 μl of the media was carefully added to wells in triplicate to avoid disturbing the pelleted cells. 100 μl of BATDA labeled cells were added to each well of the 96-well plate. Wells were saved for spontaneous release from target cells, and wells were prepared for max lysis of target cells by addition of 1% Triton-X. Monoclonal antibodies or TriNKETs against the tumor target of interest were diluted in culture media and 50 μl of diluted mAb or TriNKET was added to each well. Rested and/or activated NK cells were harvested from culture, cells were washed, and were resuspended at 10⁵-2.0×10⁶/mL in culture media depending on the desired E:T ratio. 50 μl of NK cells was added to each well of the plate to make a total of 200 μl culture volume. The plate was incubated at 37° C. with 5% CO2 for 2-3 hours before developing the assay.

After culturing for 2-3 hours, the plate was removed from the incubator and the cells were pelleted by centrifugation at 200 g for 5 minutes. 20 μl of culture supernatant was transferred to a clean microplate provided from the manufacturer and 200 μl of room temperature europium solution was added to each well. The plate was protected from the light and incubated on a plate shaker at 250 rpm for 15 minutes. Plate was read using either Victor 3 or SpectraMax i3X instruments. % Specific lysis was calculated as follows: % Specific lysis=((Experimental release−Spontaneous release)/(Maximum release−Spontaneous release))*100%.

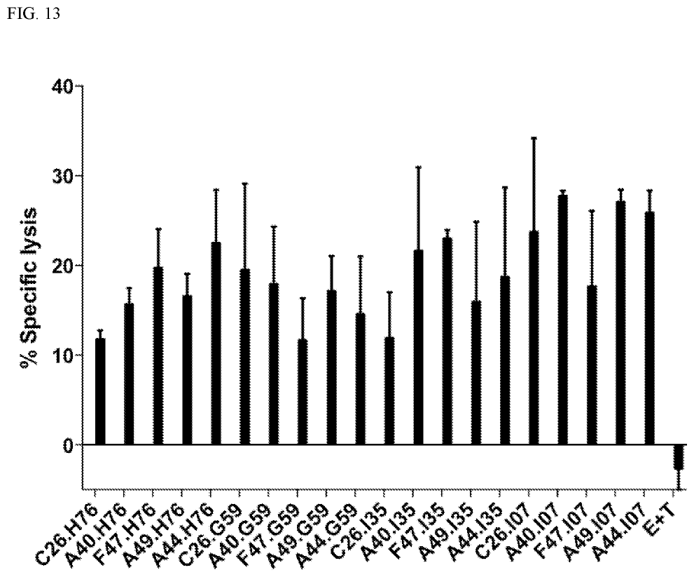

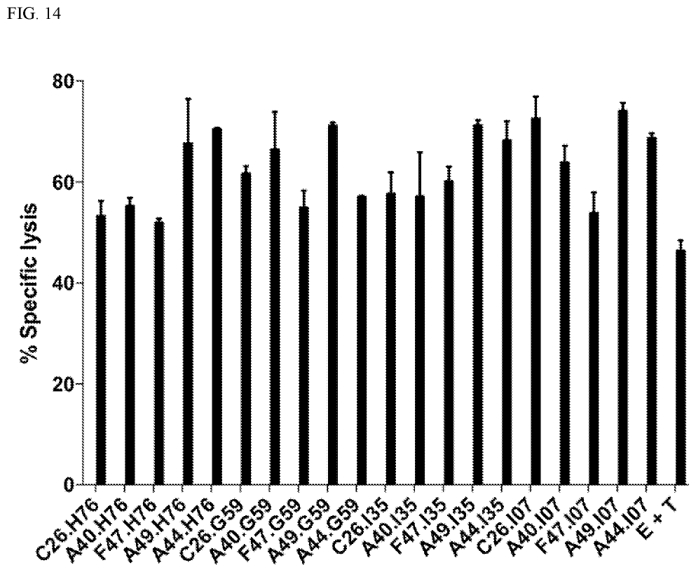

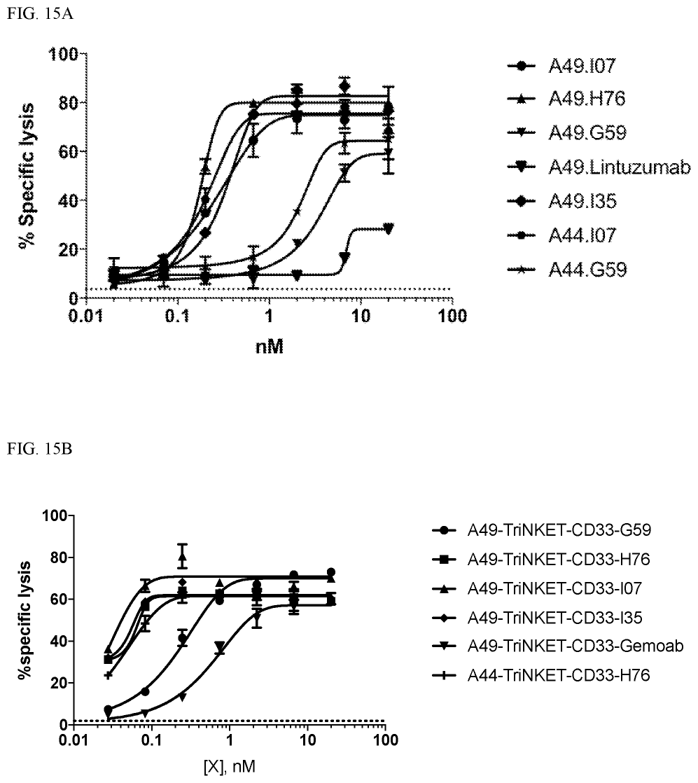

FIG. 13 and FIG. 14 show human NK cell killing of Molm-13 (FIG. 13) and THP-1 (FIG. 14) AML target cells mediated by CD33-targeting TriNKETs. Human NK cell killing of Molm-13 AML target cells mediated by CD33-targeting TriNKETs was assayed (FIG. 13). Rested NK effector cells were used with Molm-13 target cells. Activated human NK effector cells gave higher background killing, compared to rested human NK effector cells. With NK effector cells, TriNKETs were able to increase lysis against Molm-13 AML target cells. Similar activity of each TriNKET was observed with rested and activated human NK cells, as well as with Molm-13 target cells.

Human NK cell killing of THP-1 AML target cells, mediated by CD33-targeting TriNKETs was assayed. Activated human NK effector cells were used with THP-1 cells (FIG. 14), which resulted in higher background killing, compared to rested human NK effector cells. With NK effector cells, TriNKETs were able to increase lysis against THP-1 AML target cells. Similar activity of each TriNKET was observed with rested and activated human NK cells, as well as with THP-1 target cells.

Thus, with NK effector cells TriNKETs were able to increase lysis against Molm-13 (FIG. 13) and THP-1 (FIG. 14) AML target cells. Similar activity of each TriNKET was observed with rested and activated human NK cells, as well as with Molm-13 (FIG. 13) and THP-1 (FIG. 14) target cells.

FIG. 15A, FIG. 16, and FIG. 17A show KHYG-1 effector cell killing of Molm-13 (FIG. 15A), EOL-1 (FIG. 16), and THP-1 (FIG. 17A) human AML target cells respectively. KHYG-1 cells were demonstrated to express surface NKG2D, but do not express CD16. Therefore, TriNKET mediated killing here is dependent upon NKG2D mediated activation of the KHYG-1 effector cells. TriNKETs were able to mediate KHYG-1 effector cell killing of all three human AML target cell lines. Similar, TriNKET activity was demonstrated on all three target cell lines.

TriNKET mediated cytotoxicity of rested human NK cells was also tested. FIG. 15B and FIG. 17B show that TriNKETs also mediated cytotoxicity of rested human NK cells against Molm-13 (FIG. 15B) and THP-1 (FIG. 17B) human AML cells. FIG. 15B shows that TriNKETs mediated rested human NK cell killing of Molm-13 human AML cells. In FIG. 15B the rested human NK effector cell (E) to target cancer cell (T) ratio (E:T) was 10:1. The E:T ratio may reflect differences in the maximal % lysis.

TriNKETs also mediated rested human NK cell killing of THP-1 target cells, which express the high-affinity FcγRI. FIG. 17B shows that TriNKETs mediated rested human NK cell killing of THP-1 human AML cells, in which the E:T was 5:1.

### Example 8: Assessment of TriNKET Binding to Cells Expressing Human NKG2D

107 mAb was identified as a monoclonal antibody with high binding affinity to CD33. The heavy and light chain amino acid sequences of I07-F405L, an Fc variant of 107, are provided below. The I07-F405L mAb included a substitution of Leu for Phe at position 405 (under EU numbering) in the Fc CH3 domain. A Lys may be optionally included at the C-terminus of the heavy chain.

I07-F405L mAb heavy chain
[SEQ ID NO: 199]
EVQLVESGGGLVQPGGSLRLSCAASGFTFGSYWMSWVRQAPGKGLEWV
ATIKQDGSEKSYVDSVKGRFTISRDNAKNSLYLQMNSLRAEDTAVYYC
ARPLNAGELDVWGQGTMVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWES
NGQPENNYKTTPPVLDSDGSFLLYSKLTVDKSRWQQGNVFSCSVMHEA
LHNHYTQKSLSLSPG
I07-F405L mAb light chain
[SEQ ID NO: 200]
DIQMTQSPSTLSASVGDRVTITCRASQSISSWLAWYQQKPGKAPKLLI
YEASSLESGVPSRFSGSGSGTEFTLTISSLQPDDFATYYCQQSQSYPP
ITFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC

A49-F3′-TriNKET-I07, a TriNKET derivative of 107 mAb, is described in Section II—Multi-specific Binding Proteins. The amino acid sequences of this TriNKET are provided in SEQ ID NOs: 187, 189, and 190.

The ability of A49-F3′-TriNKET-I07 to bind to NKG2D was assessed using EL4 cells transduced with human NKG2D (EL4-hNKG2D). Briefly, A49-F3′-TriNKET-I07 and I07-F405L mAb were serially diluted. EL4-hNKG2D cells were incubated with the diluted TriNKET or mAb solutions. Binding of the TriNKET or mAb to the EL4 cells was detected using a fluorophore-conjugated anti-human IgG secondary antibody. The cells were analyzed by flow cytometry, and fold over background values were calculated by normalizing binding MFI to the MFI of a control group in which the cells were incubated with the secondary antibody only.

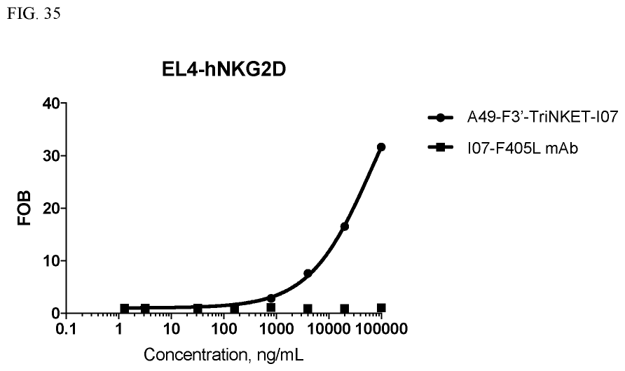

As shown in FIG. 35, I07-F405L mAb showed no binding to EL4-hNKG2D cells. A49-F3′-TriNKET-I07 demonstrated weak binding to EL4-hNKG2D cells, without reaching saturation even at the high concentrations of 100 μg/mL.

### Example 9: Assessment of TriNKET Binding to Cells Expressing Human CD33

The ability of A49-F3′-TriNKET-I07 to bind to CD33 was assessed using human cancer cell lines expressing CD33. Briefly, human AML cell lines Mv4-11 and Molm-13, which expressed CD33, were incubated with serially diluted A49-F3′-TriNKET-I07 and 107-F405L mAb solutions. Binding of A49-F3′-TriNKET-I07 or I07-F405L mAb to the AML cells was detected using a fluorophore-conjugated anti-human IgG secondary antibody. The cells were analyzed by flow cytometry, and fold over background values were calculated by normalizing binding MFI to the MFI of a control group in which the cells were incubated with the secondary antibody only.

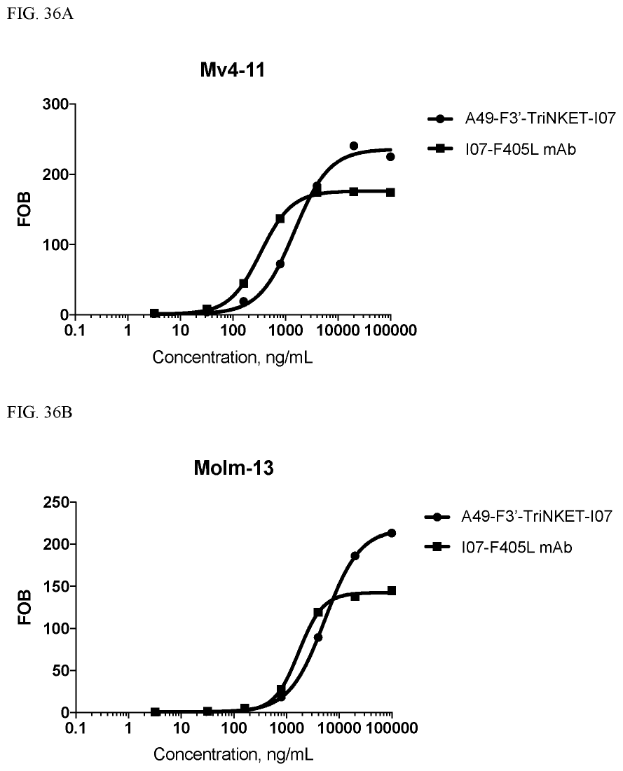

As shown in FIGS. 36A and 36B, A49-F3′-TriNKET-I07 exhibited a decrease in binding potency by three to four fold compared to I07-F405L mAb on both Molm-13 and Mv4-11 cells. A49-F3′-TriNKET-I07 was also found to bind to the cells with a higher maximum fold over background compared to I07-F405L mAb.

### Example 10: Assessment of TriNKET Internalization

Internalization of A49-F3′-TriNKET-I07 and I07-F405L mAb upon binding to CD33 was assessed using human AML cell lines EOL-1 and Molm-13, which expressed CD33 on the cell surface. Briefly, the cells were incubated with 2 μg/mL A49-F3′-TriNKET-I07 or I07-F405L mAb for 20 minutes at room temperature. The cell samples were then split into three portions. The first and second portions were placed at 37° C. for 2 hours and 24 hours, respectively, to allow antibody internalization. Then the cells were incubated with a fluorophore-conjugated anti-human IgG secondary antibody, and were fixed for flow cytometry analysis. The third portion of the cell samples, used to set the baseline level, was incubated with the fluorophore-conjugated anti-human IgG secondary antibody without incubation at 37° C. The cells were fixed after staining with the secondary antibody, and were stored at 4° C. for analysis on the following day (when the first half of the samples were ready). The amount of A49-F3′-TriNKET-I07 and I07-F405L mAb bound to the cell surface was analyzed by flow cytometry on the same day. Internalization of antibodies was calculated as: % internalization=(1−(MFI of 24-hour sample/MFI of baseline sample))×100%.

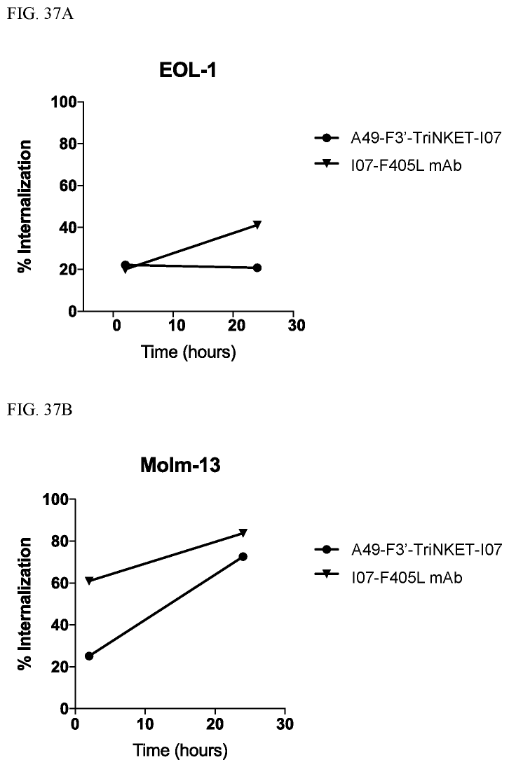

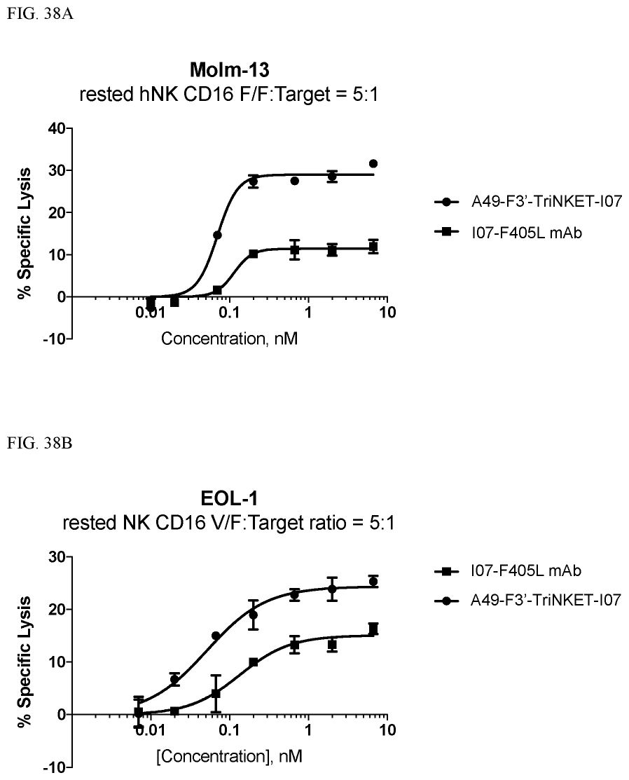

As shown in FIGS. 37A and 37B, internalization of A49-F3′-TriNKET-I07 and I07-F405L mAb after engagement of CD33 increased overtime on EOL-1 and Molm-13 cells. In both cells, I07-F405L mAb was internalized more rapidly and to a greater extent than A49-F3′-TriNKET-I07.

### Example 11: Activation of Primary NK Cells by TriNKET

The ability of A49-F3′-TriNKET-I07 to elicit cytotoxicity of primary NK cells against human AML cells was assessed using the DELFIA cytotoxicity assay. Briefly, PBMCs were isolated from human peripheral blood buffy coats using density gradient centrifugation. The isolated PBMCs were washed, and NK cells were isolated using a negative selection technique with magnetic beads. The purity of the isolated NK cells was typically >90% CD3⁻CD56⁺. The isolated NK cells were rested without cytokine overnight.

On the following day, human AML cell lines Molm-13, THP-1, and EOL-1 were harvested from culture. The AML cells were washed with HBS, and were resuspended in growth media at 10⁶ cells/mL for labeling with BATDA reagent (Perkin Elmer AD0116) following the manufacturer instructions. After labeling, the AML cells were washed three times with HBS, and were resuspended at 0.5-1.0×10⁵ cells/mL in culture media. 100 μl of BATDA labeled cells were added to each well of a 96-well plate.

The tested TriNKET or mAb was diluted in culture media, and 50 μl of diluted TriNKET or mAb was added to each well. Rested NK cells were harvested from culture, washed, and resuspended at 10⁵-2.0×10⁶ cells/mL in culture media to attain a desired E:T ratio of 5:1. 50 μl of NK cells were added to each well of the plate to make a total of 200 μl culture volume in each well. The plate was incubated at 37 C with 5% CO₂ for 2-3 hours.

After the culturing, the plate was removed from the incubator, and the cells were pelleted by centrifugation at 200×g for 5 minutes. 20 μl of culture supernatant was transferred to a clean microplate provided from the manufacturer. Supernatant from the labeled cells incubated alone was used to measure spontaneous release of TDA. Supernatant from labeled cells incubated with 1% Triton-X was used to measure maximum lysis of the target cells. Supernatant from the labeled cells prior to the 2-3 hours of incubation was used to measure the background and for quality control purposes.

200 μl of room temperature europium solution was added to each well containing culture supernatant. The plate was protected from light and incubated on a plate shaker at 250 rpm for 15 minutes. Fluorescence was measured using a Victor 3 or SpectraMax i3X instrument.

The fluorescent levels represented lysis of the target cells. The values of % specific lysis were calculated as: % specific lysis=((Experimental release −Spontaneous release)/(Maximum release −Spontaneous release))×100%.

A49-F3′-TriNKET-I07 and several monoclonal antibodies were tested in this assay. The monoclonal antibodies include I07-F405L mAb, I07-DE mAb, lintuzumab-GA, and 280-31-01(mut)-DE. I07-DE mAb is a variant of 107 mAb, with S239D and I332E substitutions in the Fc to enhance ADCC activity (bold-underlined in the sequence below). The amino acid sequence of I07-DE heavy chain is shown below, optionally with a Lys at the C-terminus.

I07-DE mAb heavy chain
[SEQ ID NO: 201]
EVQLVESGGGLVQPGGSLRLSCAASGFTFGSYWMSWVRQAPGKGLEW
VATIKQDGSEKSYVDSVKGRFTISRDNAKNSLYLQMNSLRAEDTAVY
YCARPLNAGELDVWGQGTMVTVSSASTKGPSVFPLAPSSKSTSGGTA
ALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEL
LGGPDVFLFPPKPKDTLMISRTPEVTCVVVDVSHEDPEVKFNWYVDG
VEVHNAKTKPREEQYNSTYRVVSVLTVLHQDWLNGKEYKCKVSNKAL
PAPEEKTISKAKGQPREPQVYTLPPSRDELTKNQVSLTCLVKGFYPS
DIAVEWESNGQPENNYKTTPPVLDSDGSFFLYSKLTVDKSRWQQGNV
FSCSVMHEALHNHYTQKSLSLSPG

The light chain of 107-DE mAb is identical to that of I07-F405L mAb [SEQ ID NO:200].

280-31-01(mut)-DE is a variant of antibody clone 280-31-01 (mut) disclosed in WO2012045752, with S239D and I332E substitutions in the Fc to enhance ADCC activity (bold-underlined in the sequence below). The amino acid sequences of 280-31-01(mut)-DE heavy chain and light chain are shown below, optionally with a Lys at the C-terminus of the heavy chain.

280-31-01(mut)-DE heavy chain
(SEQ ID NO: 202)
QVQLVQSGAEVKKPGSSVKVSCKASGGTFSDYAISWVRQAPGQGLEW
MGRIIPILGVADYAQKFQGRVTITADKSTRTAYMELSSLRSEDTAVY
YCARNWADAFDIWGQGTMVTVSSASTKGPSVFPLAPSSKSTSGGTAA
LGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELL
GGPDVFLFPPKPKDTLMISRTPEVTCVVVDVSHEDPEVKFNWYVDGV
EVHNAKTKPREEQYNSTYRVVSVLTVLHQDWLNGKEYKCKVSNKALP
APEEKTISKAKGQPREPQVYTLPPSREEMTKNQVSLTCLVKGFYPSD
IAVEWESNGQPENNYKTTPPVLDSDGSFFLYSKLTVDKSRWQQGNVF
SCSVMHEALHNHYTQKSLSLSPG
280-31-01(mut)-DE light chain
(SEQ ID NO: 203)
DIQLTQSPSSLSASVGDRVTITCRASQGISSVLAWYQQKPGKAPKLL
IYDASSLESGVPSRFSGSGSGTDFTLTISSLQPEDFATYYCQQFDSS
ITFGQGTKLEIKRTVAAPSVFIFPPSDEQLKSGTASVVCLLNNFYPR
EAKVQWKVDNALQSGNSQESVTEQDSKDSTYSLSSTLTLSKADYEKH
KVYACEVTHQGLSSPVTKSFNRGEC

An SNP in the CD16 gene can result in V158 or F158 variants of the human CD16 protein. CD16 with F158 is known to have reduced binding affinity of CD16 to Fc than CD16 with V158, thereby decreasing antibody-dependent cell-mediated cytotoxicity (ADCC). NK cells having CD16-F158 are therefore less responsive to CD16 stimulation than NK cells expressing CD16-V158. Indeed, as shown in FIG. 38A, I07-F405L mAb only led to a low level of killing of Molm-13 cells by NK cells that only expressed the low affinity CD16 variant (CD16^{F/F}). In comparison, A49-F3′-TriNKET-I07 mediated more potent NK cell killing with a higher specific lysis of Molm-13 cells, likely due to its additional ability to engage NK cells by NKG2D binding. Similarly, when incubated with NK cells that had this SNP in one allele (CD16^{V/F}), A49-F3′-TriNKET-I07 exhibited stronger activity in killing EOL-1 cells than I07-F405L mAb (FIG. 38B).

THP-1 cells express FcγRI, which can bind to IgG1 Fc at high affinity. The competition for Fc binding by target cells can further reduce NK cell killing. Therefore, as shown in FIGS. 38C and 38D, when THP-1 cells were incubated with CD16^{F/F} NK cells in the presence of I07-F405L or lintuzumab-GA mAb, no specific lysis of THP-1 cells was observed. Even 107-DE, an ADCC-enhancing variant of 107, failed to elicit NK cell cytotoxicity. Among the monoclonal antibodies tested, only 280-31-01(mut)-DE, an ADCC-enhancing variant of antibody clone 280-31-01 (mut) disclosed in WO2012045752, exhibited cell killing activity at high concentrations. Remarkably, A49-F3′-TriNKET-I07 mediated more potent NK cell killing with a higher specific lysis of THP-1 cells, likely due to its additional ability to engage NK cells by NKG2D binding.

### Example 12: Activation of Primary CD8⁺ T Cells by TriNKET

NKG2D is expressed on NK cells and many T cells, including CD8⁺ T cells. The ability of A49-F3′-TriNKET-I07 to elicit cytotoxicity of primary CD8⁺ T cells against human AML cells was assessed using the DELFIA cytotoxicity assay.

Briefly, human peripheral blood mononuclear cells (PBMCs) were isolated from human peripheral blood buffy coats using density gradient centrifugation. The isolated PBMCs were stimulated with 1 μg/mL Concanavalin A (ConA) at 37° C. for 18 hours. Then ConA was removed, and the PBMCs were cultured with 25 unit/mL IL-2 at 37° C. for 4 days. CD8⁺ T cells were purified using a negative selection technique with magnetic beads, then cultured in media containing 10 ng/mL IL-15 at 37° C. for 7-13 days.

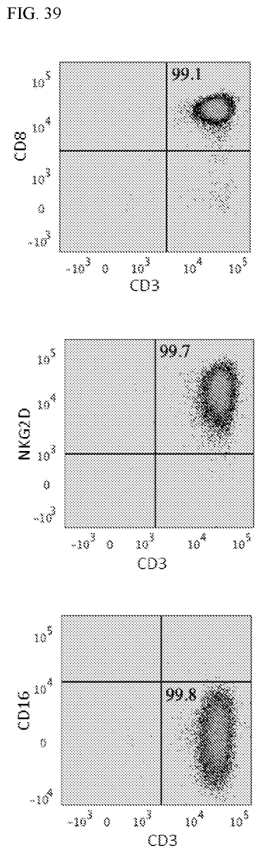

The primary human effector CD8⁺ T cells generated above were characterized for cell markers. The cells were stained with fluorophore-conjugated antibodies against CD3, CD8, NKG2D, and CD16, and analyzed by flow cytometry. As shown in FIG. 39, the isolated CD8⁺ T cells had high purity, as more than 99% of them were positive of CD3, CD8, and NKG2D expression, and were negative of CD16 expression.

To assess the ability of A49-F3′-TriNKET-I07 to elicit cytotoxicity of primary CD8⁺ T cells, Molm-13 cells were harvested from culture, washed, and resuspended in growth media at 10⁶ cells/mL. The cells were labeled with BATDA reagent (Perkin Elmer AD0116) following manufacturer instructions. After labeling, the cells were washed three times with HB S, and were resuspended at 0.5×10⁵ cells/mL in culture media. 100 μl of BATDA labeled cells were added to each well of a 96-well plate. 50 μl of serially diluted monoclonal antibody or TriNKET was added to each well.

CD8⁺ effector T cells were harvested from culture, washed, and resuspended at 5×10⁶ cells/mL in culture media. 50 μl of CD8⁺ T cells were added to each well of the plate to reach an E:T ratio of 50:1 and a total of culture volume of 200 μl. The plate was incubated at 37° C. with 5% CO₂ for 3.5 hours. After incubation, the cells were pelleted by centrifugation at 500×g for 5 minutes. 20 μl of culture supernatant was transferred to a clean microplate provided from the manufacturer. Supernatant from the labeled cells incubated alone was used to measure spontaneous release of TDA. Supernatant from labeled cells incubated with 1% Triton-X was used to measure maximum lysis of the target cells.

200 μl of room temperature europium solution was added to each well. The plate was protected from light and incubated on a plate shaker at 250 rpm for 15 minutes. Fluorescence was measured using a SpectraMax i3X instrument.

The fluorescent levels represented lysis of the target cells. The values of % specific lysis were calculated as: % specific lysis=((Experimental release −Spontaneous release)/(Maximum release −Spontaneous release))×100%.

As shown in FIGS. 40A and 40B, A49-F3′-TriNKET-I07 enhanced the cytotoxic activity of human primary CD8⁺ T cells in a dose-dependent manner. A49-F3′-TriNKET-H76, a protein described in Section II—Multi-specific Binding Proteins (having polypeptides comprising the sequences of SEQ ID NOs: 197, 189, and 190), was also active under the conditions but exhibited less potency than A49-F3′-TriNKET-I07. Monoclonal antibody I07-F405L and a non-target TriNKET did not show this activity.

### Example 13: Binding of TriNKET to Monocytes

The expression of CD33 on blood cells is assessed by flow cytometry using the method described in Example 9. Briefly, human whole blood was incubated with A49-F3′-TriNKET-I07 or human IgG1 isotype control antibody conjugated to a fluorophore. Binding of A49-F3′-TriNKET-I07 or the isotype control antibody was detected by flow cytometry. To assess the binding levels on specific types of cells, fluorophore-conjugated antibodies that bind to surface markers of NK cells, CD8⁺ T cells, CD4⁺ T cells, B cells, and monocytes were added to the incubation, and the presence or absence of binding of these antibodies were used for gating when analyzing the flow cytometry data.

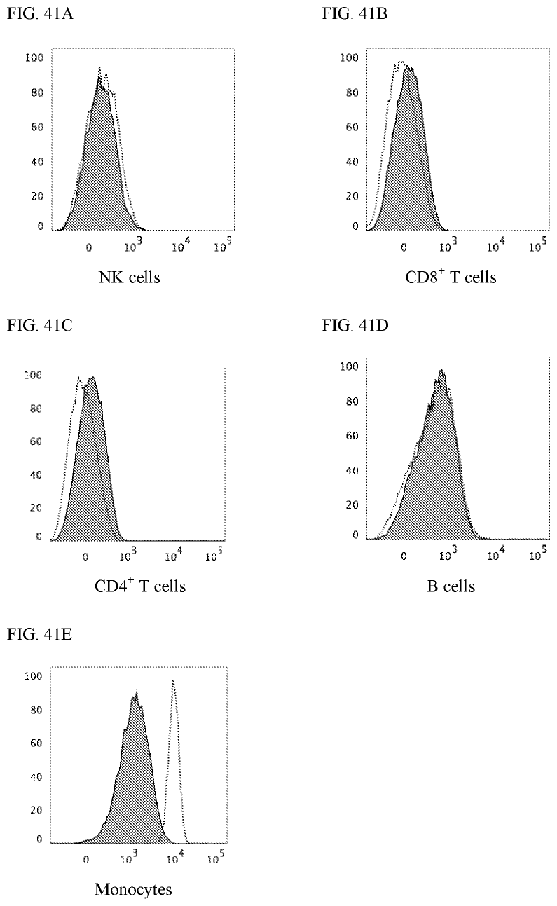

As shown in FIGS. 41A-41E, the binding of A49-F3′-TriNKET-I07 to NK cells was weak compared to a non-target human IgG1 isotype antibody control, whereas strong binding of the TriNKET to CD33⁺ monocytes was observed.

### Example 14: Long-Term NK Cell Cytotoxicity Mediated by TriNKET

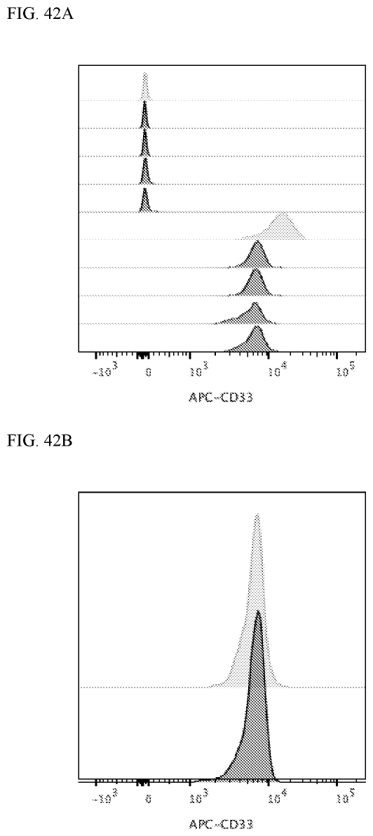

NK cells have a natural ability to sense transformed or stressed cells and kill them, but do not kill healthy cells. We tested the ability of A49-F3′-TriNKET-I07 to preserve the natural selective NK cell cytotoxicity using Molm-13 AML cells and human primary monocytes as target cells. The Molm-13 cells were obtained from DSMZ cell bank. The human primary monocytes were isolated from human peripheral blood. Briefly, PBMCs were isolated from human peripheral blood buffy coats using density gradient centrifugation. Monocytes were isolated by negative selection. CD33 expression on human primary monocytes and Molm-13 AML cells were confirmed by flow cytometry analysis (FIGS. 42A-42B).

Human primary NK cells were isolated from human peripheral blood. Briefly, PBMCs were isolated from human peripheral blood buffy coats using density gradient centrifugation. NK cells were isolated by negative selection. To distinguish target cells from NK cells in the co-culture, the target cells were fluorescently labeled. Specifically, the isolated monocytes were labeled with IncuCyte CytoLight Rapid live-cell labeling reagent according manufacturer's recommendations. The Molm-13 cells were infected with lentivirus encoding nuclear GFP, and cells with stable expression were selected with puromycin.

Isolated NK cells and target cells were mixed at an E:T ratio of 10:1 in the presence of 20 nM A49-F3′-TriNKET-I07. Non-specific activation of NK cells in the co-cultures was conducted in parallel as a positive control group for AML cell killing. The mixture was added to an Ibidi μ-slide. Time-lapse images for the phase and green channels were collected every hour, with 3 images per sample, using an IncuCyte S3 instrument. The images were analyzed using the IncuCyte S3 software. Live target cells were detected by green fluorescence, and the number of green cells at each time point was normalized to the number of green cells at time 0 from the same sample.

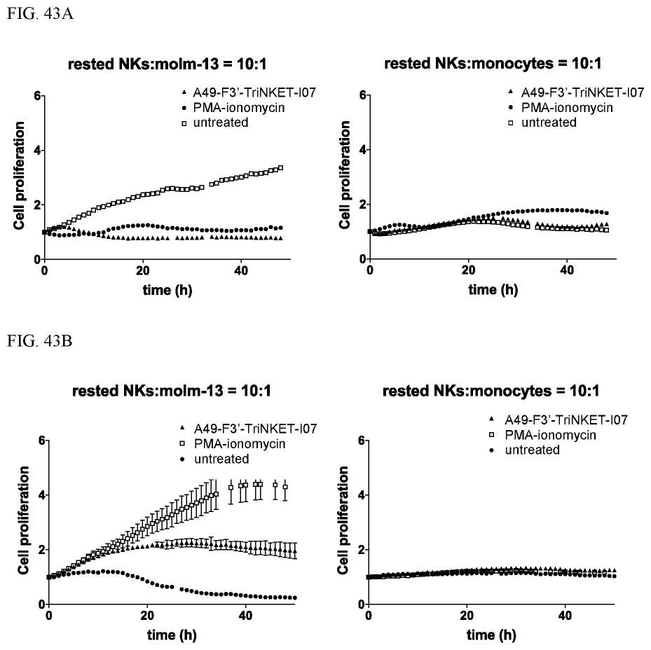

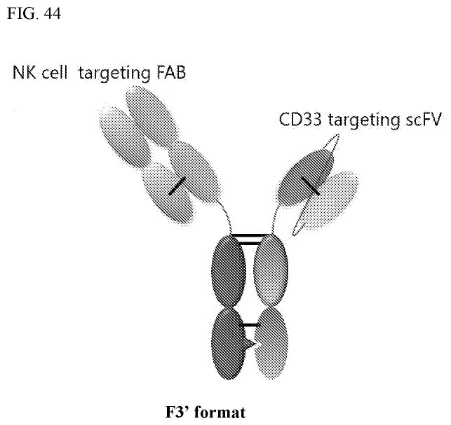

As shown in FIGS. 43A and 43B, Molm-13 AML cells were able to proliferation in the presence of the NK cells alone, but target cell outgrowth was substantially inhibited by A49-F3′-TriNKET-I07. By contrast, A49-F3′-TriNKET-I07 did not mediate human NK cell killing of normal monocytes in the long-term co-culture. The activity of A49-F3′-TriNKET-107 was similar to that of PMA+ionomycin, which also preserves the natural selectivity of NK cells. These results suggest that A49-F3′-TriNKET-I07 selectively compromised cancer cells, and potentially had a wide therapeutic window.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

What is claimed is:

1. An antibody heavy chain variable domain comprising an amino acid sequence at least 90% identical to the amino acid sequence of SEQ ID NO:9.

2. The antibody heavy chain variable domain according to claim 1, wherein the amino acid sequence comprises: a CDR1 sequence represented by the amino acid sequence of SEQ ID NO:45; a CDR2 sequence represented by the amino acid sequence of SEQ ID NO:46; and a CDR3 sequence represented by the amino acid sequence of SEQ ID NO:47.

3. An antibody heavy chain variable domain comprising an amino acid sequence at least 90% identical to the amino acid sequence of SEQ ID NO:1.

4. The antibody heavy chain variable domain according to claim 3, wherein the amino acid sequence comprises: a complementarity-determining region 1 (CDR1) sequence represented by the amino acid sequence of SEQ ID NO:21; a complementarity-determining region 2 (CDR2) sequence represented by the amino acid sequence of SEQ ID NO:22; and a complementarity-determining region 3 (CDR3) sequence represented by the amino acid sequence of SEQ ID NO:23.

5. An antibody heavy chain variable domain comprising an amino acid sequence at least 90% identical to the amino acid sequence of SEQ ID NO:3.

6. The antibody heavy chain variable domain according to claim 5, wherein the amino acid sequence comprises: a CDR1 sequence represented by the amino acid sequence of SEQ ID NO:27; a CDR2 sequence represented by the amino acid sequence of SEQ ID NO:28; and a CDR3 sequence represented by the amino acid sequence of SEQ ID NO:29.

7. An antibody heavy chain variable domain comprising an amino acid sequence at least 90% identical to the amino acid sequence of SEQ ID NO:5.

8. The antibody heavy chain variable domain according to claim 7, wherein the amino acid sequence comprises: a CDR1 sequence represented by the amino acid sequence of SEQ ID NO:33; a CDR2 sequence represented by the amino acid sequence of SEQ ID NO:34; and a CDR3 sequence represented by the amino acid sequence of SEQ ID NO:35.

9. An antibody heavy chain variable domain comprising an amino acid sequence at least 90% identical to the amino acid sequence of SEQ ID NO:7.

10. The antibody heavy chain variable domain according to claim 9, wherein the amino acid sequence comprises: a CDR1 sequence represented by the amino acid sequence of SEQ ID NO:39; a CDR2 sequence represented by the amino acid sequence of SEQ ID NO:40; and a CDR3 sequence represented by the amino acid sequence of SEQ ID NO:41.

11. An antibody heavy chain variable domain comprising an amino acid sequence at least 90% identical to the amino acid sequence of SEQ ID NO:11.

12. The antibody heavy chain variable domain according to claim 11, wherein the amino acid sequence comprises: a CDR1 sequence represented by the amino acid sequence of SEQ ID NO:51; a CDR2 sequence represented by the amino acid sequence of SEQ ID NO:52; and a CDR3 sequence represented by the amino acid sequence of SEQ ID NO:53.

13. An antibody heavy chain variable domain comprising an amino acid sequence at least 90% identical to the amino acid sequence of SEQ ID NO:13.

14. The antibody heavy chain variable domain according to claim 13, wherein the amino acid sequence comprises: a CDR1 sequence represented by the amino acid sequence of SEQ ID NO:57; a CDR2 sequence represented by the amino acid sequence of SEQ ID NO:58; and a CDR3 sequence represented by the amino acid sequence of SEQ ID NO:59.

15. An antibody heavy chain variable domain comprising an amino acid sequence at least 90% identical to the amino acid sequence of SEQ ID NO:15.

16. The antibody heavy chain variable domain according to claim 15, wherein the amino acid sequence comprises: a CDR1 sequence represented by the amino acid sequence of SEQ ID NO:63; a CDR2 sequence represented by the amino acid sequence of SEQ ID NO:64; and a CDR3 sequence represented by the amino acid sequence of SEQ ID NO:65.

17. An antibody heavy chain variable domain comprising an amino acid sequence at least 90% identical to the amino acid sequence of SEQ ID NO:17.

18. The antibody heavy chain variable domain according to claim 17, wherein the amino acid sequence comprises: a CDR1 sequence represented by the amino acid sequence of SEQ ID NO:69; a CDR2 sequence represented by the amino acid sequence of SEQ ID NO:70; and a CDR3 sequence represented by the amino acid sequence of SEQ ID NO:71.

19. An antibody heavy chain variable domain comprising an amino acid sequence at least 90% identical to the amino acid sequence of SEQ ID NO:19.

20. The antibody heavy chain variable domain according to claim 19, wherein the amino acid sequence comprises: a CDR1 sequence represented by the amino acid sequence of SEQ ID NO:75; a CDR2 sequence represented by the amino acid sequence of SEQ ID NO:76; and a CDR3 sequence represented by the amino acid sequence of SEQ ID NO:77.

21. An antibody heavy chain variable domain comprising an amino acid sequence at least 90% identical to the amino acid sequence of SEQ ID NO:266.

22. The antibody heavy chain variable domain according to claim 21, wherein the amino acid sequence comprises: a CDR1 sequence represented by the amino acid sequence of SEQ ID NO:528; a CDR2 sequence represented by the amino acid sequence of SEQ ID NO:305; and a CDR3 sequence represented by the amino acid sequence of SEQ ID NO:529.

23. An antibody heavy chain variable domain comprising an amino acid sequence at least 90% identical to the amino acid sequence of SEQ ID NO:268.

24. The antibody heavy chain variable domain according to claim 23, wherein the amino acid sequence comprises: a CDR1 sequence represented by the amino acid sequence of SEQ ID NO:530; a CDR2 sequence represented by the amino acid sequence of SEQ ID NO:311; and a CDR3 sequence represented by the amino acid sequence of SEQ ID NO:531.

25. An antibody heavy chain variable domain comprising an amino acid sequence at least 90% identical to the amino acid sequence of SEQ ID NO:270.

26. The antibody heavy chain variable domain according to claim 25, wherein the amino acid sequence comprises: a CDR1 sequence represented by the amino acid sequence of SEQ ID NO:532; a CDR2 sequence represented by the amino acid sequence of SEQ ID NO:317; and a CDR3 sequence represented by the amino acid sequence of SEQ ID NO:533.

27. An antibody heavy chain variable domain comprising an amino acid sequence at least 90% identical to the amino acid sequence of SEQ ID NO:272.

28. The antibody heavy chain variable domain according to claim 27, wherein the amino acid sequence comprises: a CDR1 sequence represented by the amino acid sequence of SEQ ID NO:534; a CDR2 sequence represented by the amino acid sequence of SEQ ID NO:323; and a CDR3 sequence represented by the amino acid sequence of SEQ ID NO:535.

29. An antibody heavy chain variable domain comprising an amino acid sequence at least 90% identical to the amino acid sequence of SEQ ID NO:274.

30. The antibody heavy chain variable domain according to claim 29, wherein the amino acid sequence comprises: a CDR1 sequence represented by the amino acid sequence of SEQ ID NO:536; a CDR2 sequence represented by the amino acid sequence of SEQ ID NO:329; and a CDR3 sequence represented by the amino acid sequence of SEQ ID NO:537.

31. An antibody heavy chain variable domain comprising an amino acid sequence at least 90% identical to the amino acid sequence of SEQ ID NO:276.

32. The antibody heavy chain variable domain according to claim 31, wherein the amino acid sequence comprises: a CDR1 sequence represented by the amino acid sequence of SEQ ID NO:538; a CDR2 sequence represented by the amino acid sequence of SEQ ID NO:335; and a CDR3 sequence represented by the amino acid sequence of SEQ ID NO:539.

33. An antibody heavy chain variable domain comprising an amino acid sequence at least 90% identical to the amino acid sequence of SEQ ID NO:278.

34. The antibody heavy chain variable domain according to claim 33, wherein the amino acid sequence comprises: a CDR1 sequence represented by the amino acid sequence of SEQ ID NO:540; a CDR2 sequence represented by the amino acid sequence of SEQ ID NO:341; and a CDR3 sequence represented by the amino acid sequence of SEQ ID NO:541.

35. An antibody heavy chain variable domain comprising an amino acid sequence at least 90% identical to the amino acid sequence of SEQ ID NO:280.

36. The antibody heavy chain variable domain according to claim 35, wherein the amino acid sequence comprises: a CDR1 sequence represented by the amino acid sequence of SEQ ID NO:542; a CDR2 sequence represented by the amino acid sequence of SEQ ID NO:347; and a CDR3 sequence represented by the amino acid sequence of SEQ ID NO:543.

37. An antibody heavy chain variable domain comprising an amino acid sequence at least 90% identical to the amino acid sequence of SEQ ID NO:282.

38. The antibody heavy chain variable domain according to claim 37, wherein the amino acid sequence comprises: a CDR1 sequence represented by the amino acid sequence of SEQ ID NO:544; a CDR2 sequence represented by the amino acid sequence of SEQ ID NO:353; and a CDR3 sequence represented by the amino acid sequence of SEQ ID NO:545.

39. An antibody heavy chain variable domain comprising an amino acid sequence at least 90% identical to the amino acid sequence of SEQ ID NO:284.

40. The antibody heavy chain variable domain according to claim 39, wherein the amino acid sequence comprises: a CDR1 sequence represented by the amino acid sequence of SEQ ID NO:546; a CDR2 sequence represented by the amino acid sequence of SEQ ID NO:359; and a CDR3 sequence represented by the amino acid sequence of SEQ ID NO:547.

41. An antibody heavy chain variable domain comprising an amino acid sequence at least 90% identical to the amino acid sequence of SEQ ID NO:286.

42. The antibody heavy chain variable domain according to claim 41, wherein the amino acid sequence comprises: a CDR1 sequence represented by the amino acid sequence of SEQ ID NO:548; a CDR2 sequence represented by the amino acid sequence of SEQ ID NO:365; and a CDR3 sequence represented by the amino acid sequence of SEQ ID NO:549.

43. An antibody heavy chain variable domain comprising an amino acid sequence at least 90% identical to the amino acid sequence of SEQ ID NO:288.

44. The antibody heavy chain variable domain according to claim 43, wherein the amino acid sequence comprises: a CDR1 sequence represented by the amino acid sequence of SEQ ID NO:550; a CDR2 sequence represented by the amino acid sequence of SEQ ID NO:371; and a CDR3 sequence represented by the amino acid sequence of SEQ ID NO:551.

45. An antibody heavy chain variable domain comprising an amino acid sequence at least 90% identical to the amino acid sequence of SEQ ID NO:290.

46. The antibody heavy chain variable domain according to claim 45, wherein the amino acid sequence comprises: a CDR1 sequence represented by the amino acid sequence of SEQ ID NO:552; a CDR2 sequence represented by the amino acid sequence of SEQ ID NO:377; and a CDR3 sequence represented by the amino acid sequence of SEQ ID NO:553.

47. An antibody heavy chain variable domain comprising an amino acid sequence at least 90% identical to the amino acid sequence of SEQ ID NO:292.

48. The antibody heavy chain variable domain according to claim 47, wherein the amino acid sequence comprises: a CDR1 sequence represented by the amino acid sequence of SEQ ID NO:554; a CDR2 sequence represented by the amino acid sequence of SEQ ID NO:383; and a CDR3 sequence represented by the amino acid sequence of SEQ ID NO:555.

49. An antibody heavy chain variable domain comprising an amino acid sequence at least 90% identical to the amino acid sequence of SEQ ID NO:294.

50. The antibody heavy chain variable domain according to claim 49, wherein the amino acid sequence comprises: a CDR1 sequence represented by the amino acid sequence of SEQ ID NO:556; a CDR2 sequence represented by the amino acid sequence of SEQ ID NO:389; and a CDR3 sequence represented by the amino acid sequence of SEQ ID NO:557.

51. An antibody heavy chain variable domain comprising an amino acid sequence at least 90% identical to the amino acid sequence of SEQ ID NO:296.

52. The antibody heavy chain variable domain according to claim 51, wherein the amino acid sequence comprises: a CDR1 sequence represented by the amino acid sequence of SEQ ID NO:558; a CDR2 sequence represented by the amino acid sequence of SEQ ID NO:395; and a CDR3 sequence represented by the amino acid sequence of SEQ ID NO:559.

53. An antibody heavy chain variable domain comprising an amino acid sequence at least 90% identical to the amino acid sequence of SEQ ID NO:298.

54. The antibody heavy chain variable domain according to claim 53, wherein the amino acid sequence comprises: a CDR1 sequence represented by the amino acid sequence of SEQ ID NO:560; a CDR2 sequence represented by the amino acid sequence of SEQ ID NO:401; and a CDR3 sequence represented by the amino acid sequence of SEQ ID NO:561.

55. An antibody heavy chain variable domain comprising an amino acid sequence at least 90% identical to the amino acid sequence of SEQ ID NO:300.

56. The antibody heavy chain variable domain according to claim 55, wherein the amino acid sequence comprises: a CDR1 sequence represented by the amino acid sequence of SEQ ID NO:562; a CDR2 sequence represented by the amino acid sequence of SEQ ID NO:407; and a CDR3 sequence represented by the amino acid sequence of SEQ ID NO:563.

57. An antibody heavy chain variable domain comprising an amino acid sequence at least 90% identical to the amino acid sequence of SEQ ID NO:302.

58. The antibody heavy chain variable domain according to claim 57, wherein the amino acid sequence comprises: a CDR1 sequence represented by the amino acid sequence of SEQ ID NO:564; a CDR2 sequence represented by the amino acid sequence of SEQ ID NO:413; and a CDR3 sequence represented by the amino acid sequence of SEQ ID NO:565.

59. An antibody heavy chain comprising an antibody heavy chain variable domain according to any one of claims 1-58 and an amino acid sequence at least 90% identical to an antibody constant region.

60. The antibody heavy chain of claim 59, wherein the antibody constant region is a human IgG constant region comprising hinge, CH2, and CH3 domains.

61. The antibody heavy chain of claim 60, wherein the antibody constant region is a human IgG constant region further comprising a CH1 domain.

62. The antibody heavy chain according to any one of claims 59-61, wherein the antibody constant region is an IgG1 constant region.

63. The antibody heavy chain according to claim 62, wherein the amino acid sequence at least 90% identical to an antibody constant region differs from the amino acid sequence of an IgG1 constant region at one or more positions selected from the group consisting of Q347, Y349, L351, S354, E356, E357, K360, Q362, S364, T366, L368, K370, N390, K392, T394, D399, S400, D401, F405, Y407, K409, T411, and K439.

64. The antibody heavy chain of claim 63, wherein the amino acid sequence at least 90% identical to an antibody constant region differs from the amino acid sequence of an IgG1 constant region by one or more substitutions selected from the group consisting of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E.

65. An antigen-binding site comprising the antibody heavy chain variable domain according to claim 1 or 2, and an antibody light chain variable domain comprising an amino acid sequence at least 90% identical to SEQ ID NO:10.

66. The antigen-binding site according to claim 65, wherein the light chain variable domain includes a CDR1 sequence identical to the amino acid sequence of SEQ ID NO:48, a CDR2 sequence identical to the amino acid sequence of SEQ ID NO:49, and a CDR3 sequence identical to the amino acid sequence of SEQ ID NO:50.

67. An antigen-binding site comprising the antibody heavy chain variable domain according to claim 3 or 4, and an antibody light chain variable domain comprising an amino acid sequence at least 90% identical to SEQ ID NO:2.

68. The antigen-binding site according to claim 67, wherein the light chain variable domain includes a CDR1 sequence identical to the amino acid sequence of SEQ ID NO:24, a CDR2 sequence identical to the amino acid sequence of SEQ ID NO:25, and a CDR3 sequence identical to the amino acid sequence of SEQ ID NO:26.

69. An antigen-binding site comprising the antibody heavy chain variable domain according to claim 5 or 6, and an antibody light chain variable domain comprising an amino acid sequence at least 90% identical to SEQ ID NO:4.

70. The antigen-binding site according to claim 69, wherein the light chain variable domain includes a CDR1 sequence identical to the amino acid sequence of SEQ ID NO:30, a CDR2 sequence identical to the amino acid sequence of SEQ ID NO:31, and a CDR3 sequence identical to the amino acid sequence of SEQ ID NO:32.

71. An antigen-binding site comprising the antibody heavy chain variable domain according to claim 7 or 8, and an antibody light chain variable domain comprising an amino acid sequence at least 90% identical to SEQ ID NO:6.

72. The antigen-binding site according to claim 71, wherein the light chain variable domain includes a CDR1 sequence identical to the amino acid sequence of SEQ ID NO:36, a CDR2 sequence identical to the amino acid sequence of SEQ ID NO:37, and a CDR3 sequence identical to the amino acid sequence of SEQ ID NO:38.

73. An antigen-binding site comprising the antibody heavy chain variable domain according to claim 9 or 10, and an antibody light chain variable domain comprising an amino acid sequence at least 90% identical to SEQ ID NO:8.

74. The antigen-binding site according to claim 73, wherein the light chain variable domain includes a CDR1 sequence identical to the amino acid sequence of SEQ ID NO:42, a CDR2 sequence identical to the amino acid sequence of SEQ ID NO:43, and a CDR3 sequence identical to the amino acid sequence of SEQ ID NO:44.

75. An antigen-binding site comprising the antibody heavy chain variable domain according to claim 11 or 12, and an antibody light chain variable domain comprising an amino acid sequence at least 90% identical to SEQ ID NO:12.

76. The antigen-binding site according to claim 75, wherein the light chain variable domain includes a CDR1 sequence identical to the amino acid sequence of SEQ ID NO:54, a CDR2 sequence identical to the amino acid sequence of SEQ ID NO:55, and a CDR3 sequence identical to the amino acid sequence of SEQ ID NO:56.

77. An antigen-binding site comprising the antibody heavy chain variable domain according to claim 13 or 14, and an antibody light chain variable domain comprising an amino acid sequence at least 90% identical to SEQ ID NO:14.

78. The antigen-binding site according to claim 77, wherein the light chain variable domain includes a CDR1 sequence identical to the amino acid sequence of SEQ ID NO:60, a CDR2 sequence identical to the amino acid sequence of SEQ ID NO:61, and a CDR3 sequence identical to the amino acid sequence of SEQ ID NO:62.

79. An antigen-binding site comprising the antibody heavy chain variable domain according to claim 15 or 16, and an antibody light chain variable domain comprising an amino acid sequence at least 90% identical to SEQ ID NO:16.

80. The antigen-binding site according to claim 79, wherein the light chain variable domain includes a CDR1 sequence identical to the amino acid sequence of SEQ ID NO:66, a CDR2 sequence identical to the amino acid sequence of SEQ ID NO:67, and a CDR3 sequence identical to the amino acid sequence of SEQ ID NO:68.

81. An antigen-binding site comprising the antibody heavy chain variable domain according to claim 17 or 18, and an antibody light chain variable domain comprising an amino acid sequence at least 90% identical to SEQ ID NO:18.

82. The antigen-binding site according to claim 81, wherein the light chain variable domain includes a CDR1 sequence identical to the amino acid sequence of SEQ ID NO:72, a CDR2 sequence identical to the amino acid sequence of SEQ ID NO:73, and a CDR3 sequence identical to the amino acid sequence of SEQ ID NO:74.

83. An antigen-binding site comprising the antibody heavy chain variable domain according to claim 19 or 20, and an antibody light chain variable domain comprising an amino acid sequence at least 90% identical to SEQ ID NO:20.

84. The antigen-binding site according to claim 83, wherein the light chain variable domain includes a CDR1 sequence identical to the amino acid sequence of SEQ ID NO:78, a CDR2 sequence identical to the amino acid sequence of SEQ ID NO:79, and a CDR3 sequence identical to the amino acid sequence of SEQ ID NO:80.

85. An antigen-binding site comprising the antibody heavy chain variable domain according to claim 21 or 22, and an antibody light chain variable domain comprising an amino acid sequence at least 90% identical to SEQ ID NO:267.

86. The antigen-binding site according to claim 85, wherein the light chain variable domain includes a CDR1 sequence identical to the amino acid sequence of SEQ ID NO:307, a CDR2 sequence identical to the amino acid sequence of SEQ ID NO:308, and a CDR3 sequence identical to the amino acid sequence of SEQ ID NO:309.

87. An antigen-binding site comprising the antibody heavy chain variable domain according to claim 23 or 24, and an antibody light chain variable domain comprising an amino acid sequence at least 90% identical to SEQ ID NO:269.

88. The antigen-binding site according to claim 87, wherein the light chain variable domain includes a CDR1 sequence identical to the amino acid sequence of SEQ ID NO:313, a CDR2 sequence identical to the amino acid sequence of SEQ ID NO:314, and a CDR3 sequence identical to the amino acid sequence of SEQ ID NO:315.

89. An antigen-binding site comprising the antibody heavy chain variable domain according to claim 25 or 26, and an antibody light chain variable domain comprising an amino acid sequence at least 90% identical to SEQ ID NO:271.

90. The antigen-binding site according to claim 89, wherein the light chain variable domain includes a CDR1 sequence identical to the amino acid sequence of SEQ ID NO:319, a CDR2 sequence identical to the amino acid sequence of SEQ ID NO:320, and a CDR3 sequence identical to the amino acid sequence of SEQ ID NO:321.

91. An antigen-binding site comprising the antibody heavy chain variable domain according to claim 27 or 28, and an antibody light chain variable domain comprising an amino acid sequence at least 90% identical to SEQ ID NO:273.

92. The antigen-binding site according to claim 91, wherein the light chain variable domain includes a CDR1 sequence identical to the amino acid sequence of SEQ ID NO:325, a CDR2 sequence identical to the amino acid sequence of SEQ ID NO:326, and a CDR3 sequence identical to the amino acid sequence of SEQ ID NO:327.

93. An antigen-binding site comprising the antibody heavy chain variable domain according to claim 29 or 30, and an antibody light chain variable domain comprising an amino acid sequence at least 90% identical to SEQ ID NO:275.

94. The antigen-binding site according to claim 93, wherein the light chain variable domain includes a CDR1 sequence identical to the amino acid sequence of SEQ ID NO:331, a CDR2 sequence identical to the amino acid sequence of SEQ ID NO:332, and a CDR3 sequence identical to the amino acid sequence of SEQ ID NO:333.

95. An antigen-binding site comprising the antibody heavy chain variable domain according to claim 31 or 32, and an antibody light chain variable domain comprising an amino acid sequence at least 90% identical to SEQ ID NO:277.

96. The antigen-binding site according to claim 95, wherein the light chain variable domain includes a CDR1 sequence identical to the amino acid sequence of SEQ ID NO:337, a CDR2 sequence identical to the amino acid sequence of SEQ ID NO:338, and a CDR3 sequence identical to the amino acid sequence of SEQ ID NO:339.

97. An antigen-binding site comprising the antibody heavy chain variable domain according to claim 33 or 34, and an antibody light chain variable domain comprising an amino acid sequence at least 90% identical to SEQ ID NO:279.

98. The antigen-binding site according to claim 97, wherein the light chain variable domain includes a CDR1 sequence identical to the amino acid sequence of SEQ ID NO:343, a CDR2 sequence identical to the amino acid sequence of SEQ ID NO:344, and a CDR3 sequence identical to the amino acid sequence of SEQ ID NO:345.

99. An antigen-binding site comprising the antibody heavy chain variable domain according to claim 35 or 36, and an antibody light chain variable domain comprising an amino acid sequence at least 90% identical to SEQ ID NO:281.

100. The antigen-binding site according to claim 99, wherein the light chain variable domain includes a CDR1 sequence identical to the amino acid sequence of SEQ ID NO:349, a CDR2 sequence identical to the amino acid sequence of SEQ ID NO:350, and a CDR3 sequence identical to the amino acid sequence of SEQ ID NO:351.

101. An antigen-binding site comprising the antibody heavy chain variable domain according to claim 37 or 38, and an antibody light chain variable domain comprising an amino acid sequence at least 90% identical to SEQ ID NO:283.

102. The antigen-binding site according to claim 101, wherein the light chain variable domain includes a CDR1 sequence identical to the amino acid sequence of SEQ ID NO:355, a CDR2 sequence identical to the amino acid sequence of SEQ ID NO:356, and a CDR3 sequence identical to the amino acid sequence of SEQ ID NO:357.

103. An antigen-binding site comprising the antibody heavy chain variable domain according to claim 39 or 40, and an antibody light chain variable domain comprising an amino acid sequence at least 90% identical to SEQ ID NO:285.

104. The antigen-binding site according to claim 103, wherein the light chain variable domain includes a CDR1 sequence identical to the amino acid sequence of SEQ ID NO:361, a CDR2 sequence identical to the amino acid sequence of SEQ ID NO:362, and a CDR3 sequence identical to the amino acid sequence of SEQ ID NO:363.

105. An antigen-binding site comprising the antibody heavy chain variable domain according to claim 41 or 42, and an antibody light chain variable domain comprising an amino acid sequence at least 90% identical to SEQ ID NO:287.

106. The antigen-binding site according to claim 105, wherein the light chain variable domain includes a CDR1 sequence identical to the amino acid sequence of SEQ ID NO:367, a CDR2 sequence identical to the amino acid sequence of SEQ ID NO:368, and a CDR3 sequence identical to the amino acid sequence of SEQ ID NO:369.

107. An antigen-binding site comprising the antibody heavy chain variable domain according to claim 43 or 44, and an antibody light chain variable domain comprising an amino acid sequence at least 90% identical to SEQ ID NO:289.

108. The antigen-binding site according to claim 107, wherein the light chain variable domain includes a CDR1 sequence identical to the amino acid sequence of SEQ ID NO:373, a CDR2 sequence identical to the amino acid sequence of SEQ ID NO:374, and a CDR3 sequence identical to the amino acid sequence of SEQ ID NO:375.

109. An antigen-binding site comprising the antibody heavy chain variable domain according to claim 45 or 46, and an antibody light chain variable domain comprising an amino acid sequence at least 90% identical to SEQ ID NO:291.

110. The antigen-binding site according to claim 109, wherein the light chain variable domain includes a CDR1 sequence identical to the amino acid sequence of SEQ ID NO:379, a CDR2 sequence identical to the amino acid sequence of SEQ ID NO:380, and a CDR3 sequence identical to the amino acid sequence of SEQ ID NO:381.

111. An antigen-binding site comprising the antibody heavy chain variable domain according to claim 47 or 48, and an antibody light chain variable domain comprising an amino acid sequence at least 90% identical to SEQ ID NO:293.

112. The antigen-binding site according to claim 111, wherein the light chain variable domain includes a CDR1 sequence identical to the amino acid sequence of SEQ ID NO:385, a CDR2 sequence identical to the amino acid sequence of SEQ ID NO:386, and a CDR3 sequence identical to the amino acid sequence of SEQ ID NO:387.

113. An antigen-binding site comprising the antibody heavy chain variable domain according to claim 49 or 50, and an antibody light chain variable domain comprising an amino acid sequence at least 90% identical to SEQ ID NO:295.

114. The antigen-binding site according to claim 113, wherein the light chain variable domain includes a CDR1 sequence identical to the amino acid sequence of SEQ ID NO:391, a CDR2 sequence identical to the amino acid sequence of SEQ ID NO:392, and a CDR3 sequence identical to the amino acid sequence of SEQ ID NO:393.

115. An antigen-binding site comprising the antibody heavy chain variable domain according to claim 51 or 52, and an antibody light chain variable domain comprising an amino acid sequence at least 90% identical to SEQ ID NO:297.

116. The antigen-binding site according to claim 115, wherein the light chain variable domain includes a CDR1 sequence identical to the amino acid sequence of SEQ ID NO:397, a CDR2 sequence identical to the amino acid sequence of SEQ ID NO:398, and a CDR3 sequence identical to the amino acid sequence of SEQ ID NO:399.

117. An antigen-binding site comprising the antibody heavy chain variable domain according to claim 53 or 54, and an antibody light chain variable domain comprising an amino acid sequence at least 90% identical to SEQ ID NO:299.

118. The antigen-binding site according to claim 117, wherein the light chain variable domain includes a CDR1 sequence identical to the amino acid sequence of SEQ ID NO:403, a CDR2 sequence identical to the amino acid sequence of SEQ ID NO:404, and a CDR3 sequence identical to the amino acid sequence of SEQ ID NO:405.

119. An antigen-binding site comprising the antibody heavy chain variable domain according to claim 55 or 56, and an antibody light chain variable domain comprising an amino acid sequence at least 90% identical to SEQ ID NO:301.

120. The antigen-binding site according to claim 119, wherein the light chain variable domain includes a CDR1 sequence identical to the amino acid sequence of SEQ ID NO:409, a CDR2 sequence identical to the amino acid sequence of SEQ ID NO:410, and a CDR3 sequence identical to the amino acid sequence of SEQ ID NO:411.

121. An antigen-binding site comprising the antibody heavy chain variable domain according to claim 57 or 58, and an antibody light chain variable domain comprising an amino acid sequence at least 90% identical to SEQ ID NO:303.

122. The antigen-binding site according to claim 121, wherein the light chain variable domain includes a CDR1 sequence identical to the amino acid sequence of SEQ ID NO:415, a CDR2 sequence identical to the amino acid sequence of SEQ ID NO:416, and a CDR3 sequence identical to the amino acid sequence of SEQ ID NO:417.

123. A protein comprising the antigen-binding site according to any one of claims 65-122, wherein the antigen-binding site binds to human CD33.

124. The protein of claim 123, wherein the protein further comprises a second antigen binding site same or different from the antigen-binding site that binds to human CD33.

125. The protein of claim 123 or 124, further comprising an antibody constant region.

126. The protein of claim 125, wherein the antibody constant region is capable of binding to CD16 and comprises two polypeptide chains, each of which comprises a hinge, CH2 and CH3 domain.

127. The protein of claim 125 or 126, wherein each of the two polypeptide chains of the antibody constant region comprises amino acid sequence at least 90% identical to human IgG1 constant region.

128. The protein of claim 127, the amino acid sequence of one polypeptide chain of the antibody constant region differs from the amino acid sequence of an IgG1 constant region at one or more positions selected from the group consisting of Q347, Y349, L351, S354, E356, E357, K360, Q362, S364, T366, L368, K370, K392, T394, D399, S400, D401, F405, Y407, K409, T411 and K439; and the amino acid sequence of the other polypeptide chain of the antibody constant region differs from the amino acid sequence of an IgG1 constant region at one or more positions selected from the group consisting of Q347, Y349, L351, S354, E356, E357, S364, T366, L368, K370, N390, K392, T394, D399, D401, F405, Y407, K409, T411 and K439.

129. The protein of claim 127, wherein the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

130. The protein of claim 127, wherein the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

131. The protein of claim 127, wherein the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

132. The protein of claim 127, wherein the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

133. The protein of claim 127, wherein the amino acid sequence of one polypeptide chain of the antibody constant region differs from the amino acid sequence of an IgG1 constant region at one or more positions selected from the group consisting of L351, D399, 5400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

134. The protein of claim 127, wherein the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, 5400 and Y407.

135. The protein of claim 127, wherein the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

136. The protein of claim 127, wherein the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

137. The protein of claim 127, wherein the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

138. The protein of claim 127, wherein the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

139. The protein of claim 127, wherein the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

140. The protein of claim 127, wherein the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

141. The protein of claim 127, wherein the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

142. The protein of claim 127, wherein the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

143. The protein of claim 127, wherein the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by 0347R, D399V and F405T substitutions.

144. The protein of claim 127, wherein the amino acid sequence of one polypeptide chain of the antibody constant region differs from the amino acid sequence of an IgG1 constant region by 0347R, D399V and F405T substitutions and wherein the amino acid sequence of the other polypeptide chain of the antibody constant region differs from the amino acid sequence of an IgG1 constant region by K360E and K409W substitutions.

145. The protein of claim 127, wherein the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

146. The protein of claim 127, wherein the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

147. The protein of claim 127, wherein the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

148. The protein of claim 127, wherein the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

149. A protein comprising an antigen-binding site that competes for binding to human and cynomolgus CD33 with an antibody comprising an antibody heavy chain variable domain and an antibody light chain variable domain having the amino acid sequences of SEQ ID NOs: 1 and 2; 3 and 4; 5 and 6; 7 and 8; 9 and 10; 11 and 12; 13 and 14; 15 and 16; 17 and 18; 19 and 20; 266 and 267; 268 and 269; 270 and 271; 272 and 273; 274 and 275; 276 and 277; 278 and 279; 280 and 281; 282 and 283; 284 and 285; 286 and 287; 288 and 289; 290 and 291; 292 and 293; 294 and 295; 296 and 297; 298 and 299; 300 and 301; or 302 and 303, respectively.

150. A formulation comprising a protein of any one of claims 123-149, and a pharmaceutically acceptable carrier.

151. An isolated nucleic acid encoding a chimeric antigen receptor (CAR), wherein the nucleic acid comprises a nucleic acid sequence that encodes a CD33-binding scFv comprising a sequence at least 90% identical to an amino acid sequence selected from SEQ ID NOs:188, 198, 206-223, and 447-484; a nucleic acid sequence encoding a transmembrane domain; and a nucleic acid sequence encoding an intracellular signaling domain.

152. An isolated nucleic acid encoding a chimeric antigen receptor (CAR), wherein the nucleic acid comprises a nucleic acid sequence that encodes a CD33-binding scFv comprising a sequence at least 95% identical to an amino acid sequence selected from SEQ ID NOs:188, 198, 206-223, and 447-484; a nucleic acid sequence encoding a transmembrane domain; and a nucleic acid sequence encoding an intracellular signaling domain.

153. An isolated nucleic acid encoding a chimeric antigen receptor (CAR), wherein the nucleic acid comprises a nucleic acid sequence that encodes a CD33-binding scFv comprising a sequence at least 99% identical to an amino acid sequence selected from SEQ ID NOs:188, 198, 206-223, and 447-484; a nucleic acid sequence encoding a transmembrane domain; and a nucleic acid sequence encoding an intracellular signaling domain.

154. The isolated nucleic acid sequence of any one of claims 151-153, wherein the transmembrane domain is selected from the transmembrane regions of the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, CD152, and CD154.

155. The isolated nucleic acid of any one of claims 151-154, wherein the CD33-binding scFv is connected to the transmembrane domain by a hinge region.

156. The isolated nucleic acid of any one of claims 151-155, wherein the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

157. The isolated nucleic acid of any one of claims 151-156, wherein the intracellular signaling domain further comprises a costimulatory signaling domain comprising a functional signaling domain of a costimulatory receptor.

158. The isolated nucleic acid of claim 157, wherein the costimulatory receptor is selected from the group consisting of OX40, CD27, CD28, CD30, CD40, PD-1, CD2, CD7, CD258, NKG2C, B7-H3, a ligand that binds to CD83, LFA-1 (CD11a/CD18), ICOS and 4-1BB (CD137), or any combination thereof.

159. An expression vector comprising the isolated nucleic acid of any one of claims 111-118.

160. A chimeric antigen receptor (CAR), wherein the CAR comprises a CD33-binding scFv comprising amino acid sequence at least 90% identical to an amino acid sequence selected from SEQ ID NOs:188, 198, 206-223, and 447-484; a transmembrane domain; and an intracellular signaling domain.

161. The CAR of claim 160, wherein the transmembrane domain is selected from the transmembrane regions of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD152, and CD154.

162. The CAR of claim 160 or 161, wherein the CD33-binding scFv is connected to the transmembrane domain by a hinge region.

163. The CAR of any one of claims 160-162, wherein the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

164. The CAR of any one of claims 160-163, wherein the intracellular signaling domain further comprises a costimulatory signaling domain comprising a functional signaling domain of a costimulatory receptor.

165. The CAR of claim 164, wherein the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

166. An immune effector cell comprising the nucleic acid of any one of claims 151-158.

167. An immune effector cell comprising the vector of claim 159.

168. An immune effector cell expressing the CAR of any one of claims 160-165.

169. The immune effector cell of any one of claims 166-168, wherein the immune effector cell is a T cell.

170. The immune effector cell of claim 169, wherein the T cell is a CD8⁺ T cell, a CD4⁺ T cell, or an NKT cell.

171. The immune effector cell of any one of claims 166-168, wherein the immune effector cell is an NK cell.

172. A CD33/CD3-directed bispecific T-cell engager comprising a protein comprising a sequence at least 90% identical to an amino acid sequence selected from SEQ ID NOs:188, 198, 206-223, and 447-484.

173. A CD33/CD3-directed bispecific T-cell engager comprising a protein comprising a sequence at least 95% identical to an amino acid sequence selected from SEQ ID NOs:188, 198, 206-223, and 447-484.

174. A CD33/CD3-directed bispecific T-cell engager comprising a protein comprising a sequence at least 99% identical to an amino acid sequence selected from SEQ ID NOs:188, 198, 206-223, and 447-484.

175. An isolated nucleic acid encoding the CD33/CD3-directed bispecific T-cell engager of any one of claims 172-174.

176. An antibody-drug conjugate comprising a protein comprising a sequence at least 90% identical to an amino acid sequence selected from SEQ ID NOs:188, 198, 206-223, and 447-484.

177. An antibody-drug conjugate comprising a protein comprising a sequence at least 95% identical to an amino acid sequence selected from SEQ ID NOs:188, 198, 206-223, and 447-484.

178. An antibody-drug conjugate comprising a protein comprising a sequence at least 99% identical to an amino acid sequence selected from SEQ ID NOs:188, 198, 206-223, and 447-484.

179. The antibody-drug conjugate of any one of claims 176-178, further comprising a drug moiety selected from auristatin, N-acetyl-γ calicheamicin, maytansinoid, pyrrolobenzodiazepine, and SN-38.

180. An immunocytokine comprising a sequence at least 90% identical to an amino acid sequence selected from SEQ ID NOs:188, 198, 206-223, and 447-484, connected to a cytokine.

181. An immunocytokine comprising a sequence at least 95% identical to an amino acid sequence selected from SEQ ID NOs:188, 198, 206-223, and 447-484, connected to a cytokine.

182. An immunocytokine comprising a sequence at least 99% identical to an amino acid sequence selected from SEQ ID NOs:188, 198, 206-223, and 447-484, connected to a cytokine.

183. The immunocytokine of any one of claims 170-172, wherein the cytokine is selected from IL-2, IL-4, IL-10, IL-12, IL-15, TNF, and IFNα.

184. An isolated nucleic acid encoding an immunocytokine of any one of claims 170-173.

185. A method of treating a CD33-expressing cancer, the method comprising administering an effective amount of the protein according to any one of claims 123-149, the formulation according to claim 150, the immune effector cell according to any one of claims 166-171, the CD33/CD3-directed bispecific T-cell engager according to any one of claims 172-174, the antibody-drug conjugate according to any one of claims 176-179, or the immunocytokine according to any one of claims 180-183 to a subject in need thereof.

186. The method of claim 185, wherein the cancer is selected from the group consisting of acute myeloid leukemia (AML), myelodysplastic syndrome (MDS), chronic lymphocytic leukemia (CIVIL), myeloid blast crisis of CIVIL, acute lymphoblastic leukemia (ALL), acute lymphoblastic lymphoma, myeloproliferative neoplasms (MPNs), lymphoma, non-Hodgkin lymphomas, and classical Hodgkin lymphoma.

187. The method of claim 186, wherein the AML is selected from undifferentiated acute myeloblastic leukemia, acute myeloblastic leukemia with minimal maturation, acute myeloblastic leukemia with maturation, acute promyelocytic leukemia (APL), acute myelomonocytic leukemia, acute myelomonocytic leukemia with eosinophilia, acute monocytic leukemia, acute erythroid leukemia, acute megakaryoblastic leukemia (AMKL), acute basophilic leukemia, acute panmyelosis with fibrosis, and blastic plasmacytoid dendritic cell neoplasm (BPDCN).

188. The method of claim 186 or 187, wherein the AML is characterized by expression of CLL-1 on the AML leukemia stem cells (LSCs).

189. The method of claim 188, wherein the LSCs further express a membrane marker selected from CD34, CD38, CD123, TIM3, CD25, CD32, and CD96.

190. The method of any one of claims 186-189, wherein the AML is a minimal residual disease (MRD).

191. The method of claim 190, wherein the MRD is characterized by the presence or absence of a mutation selected from FLT3-ITD ((Fms-like tyrosine kinase 3)-internal tandem duplications (ITD)), NPM1 (Nucleophosmin 1), DNMT3A (DNA methyltransferase gene DNMT3A), and IDH (Isocitrate dehydrogenase 1 and 2 (IDH1 and IDH2)).

192. The method of claim 186, wherein the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q), and MDS, unclassified (MDS-U).

193. The method of claim 186 or 192, wherein the MDS is a primary MDS or a secondary MDS.

194. The method of claim 186, wherein the ALL is selected from B-cell acute lymphoblastic leukemia (B-ALL) and T-cell acute lymphoblastic leukemia (T-ALL).

195. The method of claim 186, wherein the MPN is selected from polycythaemia vera, essential thrombocythemia (ET), and myelofibrosis.

196. The method of claim 186, wherein the non-Hodgkin lymphoma is selected from B-cell lymphoma and T-cell lymphoma.

197. The method of claim 186, wherein the lymphoma is selected from chronic lymphocytic leukemia (CLL), lymphoblastic lymphoma (LPL), diffuse large B-cell lymphoma (DLBCL), Burkitt lymphoma (BL), primary mediastinal large B-cell lymphoma (PMBL), follicular lymphoma, mantle cell lymphoma, hairy cell leukemia, plasma cell myeloma (PCM) or multiple myeloma (MM), mature T/NK neoplasms, and histiocytic neoplasms.


SEQUENCE LISTING (599 sequences)

SEQ ID NO: 1 - protein, 128 aa, Artificial Sequence
  Description of Artificial Sequence Synthetic polypeptide
EVQLVESGGGLVQPGGSLRLSCAASGFTFSSYGMSWVRQAPGKGLEWVANIKQDGSEKYYVDSVKGRFTISRDNAKNSLYLQMNSLRAEDTAVYYCAREGGPYYDSSGYFVYYGMDVWGQGTTVTVSS

SEQ ID NO: 2 - protein, 106 aa, Artificial Sequence
  Description of Artificial Sequence Synthetic polypeptide
DIQMTQSPSTLSASVGDRVTITCRASQSISSWLAWYQQKPGKAPKLLIYDASSLESGVPSRFSGSGSGTEFTLTISSLQPDDFATYYCQQYESFPTFGGGTKVEIK

SEQ ID NO: 3 - protein, 118 aa, Artificial Sequence
  Description of Artificial Sequence Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARPLNAGELDVWGQGTMVTVSS

SEQ ID NO: 4 - protein, 107 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQLESYPLTFGGGTKVEIK

SEQ ID NO: 5 - protein, 128 aa, Artificial Sequence
  Description of Artificial Sequence Synthetic polypeptide
EVQLLESGGGLVQPGGSLRLSCAASGFTFSKYTMSWVRQAPGKGLEWVSAIVGSGESTYFADSVKGRFTISRDNSKNTLYLQMNSLRAEDTAVYYCAREGGPYYDSSGYFVYYGMDVWGQGTTVTVSS

SEQ ID NO: 6 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDDLPTFGGGTKVEIK

SEQ ID NO: 7 - protein, 123 aa, Artificial Sequence
  Description of Artificial Sequence Synthetic polypeptide
QVQLVQSGAEVKKPGASVKVSCKASGYTFSDYYMHWVRQAPGQGLEWMGMINPSWGSTSYAQKFQGRVTMTRDTSTSTVYMELSSLRSEDTAVYYCAREAADGFVGERYFDLWGRGTLVTVSS

SEQ ID NO: 8 - protein, 112 aa, Artificial Sequence
  Description of Artificial Sequence Synthetic polypeptide
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGGGTKVEIK

SEQ ID NO: 9 - protein, 118 aa, Artificial Sequence
  Description of Artificial Sequence Synthetic polypeptide
EVQLVESGGGLVQPGGSLRLSCAASGFTFGSYWMSWVRQAPGKGLEWVATIKQDGSEKSYVDSVKGRFTISRDNAKNSLYLQMNSLRAEDTAVYYCARPLNAGELDVWGQGTMVTVSS

SEQ ID NO: 10 - protein, 108 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGGGTKVEIK

SEQ ID NO: 11 - protein, 118 aa, Artificial Sequence
  Description of Artificial Sequence Synthetic polypeptide
EVQLVESGGGLVQPGGSLRLSCAASGFTFPSYWMSWVRQAPGKGLEWVATIKRDGSEKGYVDSVKGRFTISRDNAKNSLYLQMNSLRAEDTAVYYCARPLNAGELDVWGQGTMVTVSS

SEQ ID NO: 12 - protein, 108 aa, Artificial Sequence; identical to SEQ ID NO: 10
  Description of Artificial Sequence Synthetic polypeptide

SEQ ID NO: 13 - protein, 120 aa, Artificial Sequence
  Description of Artificial Sequence Synthetic polypeptide
QVQLVQSGAEVKKPGASVKVSCKASGYTFGTYYMHWVRQAPGQGLEWMGIINPSRGSTVYAQKFQGRVTMTRDTSTSTVYMELSSLRSEDTAVYYCARGAGYDDEDMDVWGKGTTVTVSS

SEQ ID NO: 14 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGGGTKVEIK

SEQ ID NO: 15 - protein, 128 aa, Artificial Sequence
  Description of Artificial Sequence Synthetic polypeptide
EVQLVESGGGLVKPGGSLRLSCAASGFTFSSYAMSWVRQAPGKGLEWVSSISSSSEGIYYADSVKGRFTISRDNAKNSLYLQMNSLRAEDTAVYYCAREGGPYYDSSGYFVYYGMDVWGQGTTVTVSS

SEQ ID NO: 16 - protein, 106 aa, Artificial Sequence
  Description of Artificial Sequence Synthetic polypeptide
DIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGGGTKVEIK

SEQ ID NO: 17 - protein, 123 aa, Artificial Sequence
  Description of Artificial Sequence Synthetic polypeptide
EVQLVESGGGLVQPGGSLRLSCAASGFTFSSYWMSWVRQAPGKGLEWVANINTDGSEVYYVDSVKGRFTISRDNAKNSLYLQMNSLRAEDTAVYYCARDVGPGIAYQGHFDYWGQGTLVTVSS

SEQ ID NO: 18 - protein, 107 aa, Artificial Sequence
  Description of Artificial Sequence Synthetic polypeptide
DIQMTQSPSSLSASVGDRVTITCRASQVIYSYLNWYQQKPGKAPKLLIYAASSLKSGVPSRFSGSGSGTDFTLTISSLQPEDFATYYCQQVYDTPLTFGGGTKVEIK

SEQ ID NO: 19 - protein, 120 aa, Artificial Sequence
  Description of Artificial Sequence Synthetic polypeptide
QLQLQESGPGLVKPSETLSLTCTVSGGSISSTDYYWGWIRQPPGKGLEWIGSIGYSGTYYNPSLKSRVTISVDTSKNQFSLKLSSVTAADTAVYYCARETAHDVHGMDVWGQGTTVTVSS

SEQ ID NO: 20 - protein, 106 aa, Artificial Sequence
  Description of Artificial Sequence Synthetic polypeptide
EIVLTQSPATLSLSPGERATLSCRASHSVYSYLAWYQQKPGQAPRLLIYDASNRATGIPARFSGSGSGTDFTLTISSLEPEDFAVYYCQQYDNLPTFGGGTKVEIK

SEQ ID NO: 21 - protein, 9 aa, Artificial Sequence
  Description of Artificial Sequence Synthetic peptide
FTFSSYGMS

SEQ ID NO: 22 - protein, 17 aa, Artificial Sequence
  Description of Artificial Sequence Synthetic peptide
NIKQDGSEKYYVDSVKG

SEQ ID NO: 23 - protein, 21 aa, Artificial Sequence
  Description of Artificial Sequence Synthetic peptide
AREGGPYYDSSGYFVYYGMDV

SEQ ID NO: 24 - protein, 11 aa, Artificial Sequence
  Description of Artificial Sequence Synthetic peptide
RASQSISSWLA

SEQ ID NO: 25 - protein, 7 aa, Artificial Sequence
  Description of Artificial Sequence Synthetic peptide
DASSLES

SEQ ID NO: 26 - protein, 8 aa, Artificial Sequence
  Description of Artificial Sequence Synthetic peptide
QQYESFPT

SEQ ID NO: 27 - protein, 9 aa, Artificial Sequence
  Description of Artificial Sequence Synthetic peptide
FTFSSYWMS

SEQ ID NO: 28 - protein, 17 aa, Artificial Sequence
  Description of Artificial Sequence Synthetic peptide
NIKQDGSEKYYVDSVKG

SEQ ID NO: 29 - protein, 11 aa, Artificial Sequence
  Description of Artificial Sequence Synthetic peptide
ARPLNAGELDV

SEQ ID NO: 30 - protein, 11 aa, Artificial Sequence
  Description of Artificial Sequence Synthetic peptide
RASQSISSWLA

SEQ ID NO: 31 - protein, 7 aa, Artificial Sequence
  Description of Artificial Sequence Synthetic peptide
EASSLES

SEQ ID NO: 32 - protein, 9 aa, Artificial Sequence
  Description of Artificial Sequence Synthetic peptide
QQLESYPLT

SEQ ID NO: 33 - protein, 9 aa, Artificial Sequence
  Description of Artificial Sequence Synthetic peptide
FTFSKYTMS

SEQ ID NO: 34 - protein, 17 aa, Artificial Sequence
  Description of Artificial Sequence Synthetic peptide
AIVGSGESTYFADSVKG

SEQ ID NO: 35 - protein, 21 aa, Artificial Sequence
  Description of Artificial Sequence Synthetic peptide
AREGGPYYDSSGYFVYYGMDV

SEQ ID NO: 36 - protein, 11 aa, Artificial Sequence
  Description of Artificial Sequence Synthetic peptide
RASQSISSWLA

SEQ ID NO: 37 - protein, 7 aa, Artificial Sequence
  Description of Artificial Sequence Synthetic peptide
KASSLES

SEQ ID NO: 38 - protein, 8 aa, Artificial Sequence
  Description of Artificial Sequence Synthetic peptide
QQYDDLPT

SEQ ID NO: 39 - protein, 9 aa, Artificial Sequence
  Description of Artificial Sequence Synthetic peptide
YTFSDYYMH

SEQ ID NO: 40 - protein, 17 aa, Artificial Sequence
  Description of Artificial Sequence Synthetic peptide
MINPSWGSTSYAQKFQG

SEQ ID NO: 41 - protein, 16 aa, Artificial Sequence
  Description of Artificial Sequence Synthetic peptide
AREAADGFVGERYFDL

SEQ ID NO: 42 - protein, 16 aa, Artificial Sequence
  Description of Artificial Sequence Synthetic peptide
RSSQSLLYSNGYNYLD

SEQ ID NO: 43 - protein, 7 aa, Artificial Sequence
  Description of Artificial Sequence Synthetic peptide
LGSNRAS

SEQ ID NO: 44 - protein, 9 aa, Artificial Sequence
  Description of Artificial Sequence Synthetic peptide
MQDVALPIT

SEQ ID NO: 45 - protein, 9 aa, Artificial Sequence
  Description of Artificial Sequence Synthetic peptide
FTFGSYWMS

SEQ ID NO: 46 - protein, 17 aa, Artificial Sequence
  Description of Artificial Sequence Synthetic peptide
TIKQDGSEKSYVDSVKG

SEQ ID NO: 47 - protein, 11 aa, Artificial Sequence
  Description of Artificial Sequence Synthetic peptide
ARPLNAGELDV

SEQ ID NO: 48 - protein, 11 aa, Artificial Sequence
  Description of Artificial Sequence Synthetic peptide
RASQSISSWLA

SEQ ID NO: 49 - protein, 7 aa, Artificial Sequence
  Description of Artificial Sequence Synthetic peptide
EASSLES

SEQ ID NO: 50 - protein, 10 aa, Artificial Sequence
  Description of Artificial Sequence Synthetic peptide
QQSQSYPPIT

SEQ ID NO: 51 - protein, 9 aa, Artificial Sequence
  Description of Artificial Sequence Synthetic peptide
FTFPSYWMS

SEQ ID NO: 52 - protein, 17 aa, Artificial Sequence
  Description of Artificial Sequence Synthetic peptide
TIKRDGSEKGYVDSVKG

SEQ ID NO: 53 - protein, 11 aa, Artificial Sequence
  Description of Artificial Sequence Synthetic peptide
ARPLNAGELDV

SEQ ID NO: 54 - protein, 11 aa, Artificial Sequence
  Description of Artificial Sequence Synthetic peptide
RASQSISSWLA

SEQ ID NO: 55 - protein, 7 aa, Artificial Sequence
  Description of Artificial Sequence Synthetic peptide
EASSLES

SEQ ID NO: 56 - protein, 10 aa, Artificial Sequence
  Description of Artificial Sequence Synthetic peptide
QQSQSYPPIT

SEQ ID NO: 57 - protein, 9 aa, Artificial Sequence
  Description of Artificial Sequence Synthetic peptide
YTFGTYYMH

SEQ ID NO: 58 - protein, 17 aa, Artificial Sequence
  Description of Artificial Sequence Synthetic peptide
IINPSRGSTVYAQKFQG

SEQ ID NO: 59 - protein, 13 aa, Artificial Sequence
  Description of Artificial Sequence Synthetic peptide
ARGAGYDDEDMDV

SEQ ID NO: 60 - protein, 11 aa, Artificial Sequence
  Description of Artificial Sequence Synthetic peptide
RASQGIDSWLA

SEQ ID NO: 61 - protein, 7 aa, Artificial Sequence
  Description of Artificial Sequence Synthetic peptide
AASSLQS

SEQ ID NO: 62 - protein, 9 aa, Artificial Sequence
  Description of Artificial Sequence Synthetic peptide
QQAHSYPLT

SEQ ID NO: 63 - protein, 9 aa, Artificial Sequence
  Description of Artificial Sequence Synthetic peptide
FTFSSYAMS

SEQ ID NO: 64 - protein, 17 aa, Artificial Sequence
  Description of Artificial Sequence Synthetic peptide
SISSSSEGIYYADSVKG

SEQ ID NO: 65 - protein, 21 aa, Artificial Sequence
  Description of Artificial Sequence Synthetic peptide
AREGGPYYDSSGYFVYYGMDV

SEQ ID NO: 66 - protein, 11 aa, Artificial Sequence
  Description of Artificial Sequence Synthetic peptide
RASNSISSWLA

SEQ ID NO: 67 - protein, 7 aa, Artificial Sequence
  Description of Artificial Sequence Synthetic peptide
EASSTKS

SEQ ID NO: 68 - protein, 8 aa, Artificial Sequence
  Description of Artificial Sequence Synthetic peptide
QQYDDLPT

SEQ ID NO: 69 - protein, 9 aa, Artificial Sequence
  Description of Artificial Sequence Synthetic peptide
FTFSSYWMS

SEQ ID NO: 70 - protein, 17 aa, Artificial Sequence
  Description of Artificial Sequence Synthetic peptide
NINTDGSEVYYVDSVKG

SEQ ID NO: 71 - protein, 16 aa, Artificial Sequence
  Description of Artificial Sequence Synthetic peptide
ARDVGPGIAYQGHFDY

SEQ ID NO: 72 - protein, 11 aa, Artificial Sequence
  Description of Artificial Sequence Synthetic peptide
RASQVIYSYLN

SEQ ID NO: 73 - protein, 7 aa, Artificial Sequence
  Description of Artificial Sequence Synthetic peptide
AASSLKS

SEQ ID NO: 74 - protein, 9 aa, Artificial Sequence
  Description of Artificial Sequence Synthetic peptide
QQVYDTPLT

SEQ ID NO: 75 - protein, 11 aa, Artificial Sequence
  Description of Artificial Sequence Synthetic peptide
GSISSTDYYWG

SEQ ID NO: 76 - protein, 15 aa, Artificial Sequence
  Description of Artificial Sequence Synthetic peptide
SIGYSGTYYNPSLKS

SEQ ID NO: 77 - protein, 13 aa, Artificial Sequence
  Description of Artificial Sequence Synthetic peptide
ARETAHDVHGMDV

SEQ ID NO: 78 - protein, 11 aa, Artificial Sequence
  Description of Artificial Sequence Synthetic peptide
RASHSVYSYLA

SEQ ID NO: 79 - protein, 7 aa, Artificial Sequence
  Description of Artificial Sequence Synthetic peptide
DASNRAT

SEQ ID NO: 80 - protein, 8 aa, Artificial Sequence
  Description of Artificial Sequence Synthetic peptide
QQYDNLPT

SEQ ID NO: 81 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 82 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 83 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 84 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 85 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 86 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 87 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 88 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 89 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 90 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 91 - protein, 122 aa, Artificial Sequence
  Description of Artificial Sequence Synthetic polypeptide
  VARIANT (102)..(102): Met, Leu, Ile, Val, Gln, or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 92 - protein, 15 aa, Artificial Sequence
  Description of Artificial Sequence Synthetic p eptide
  VARIANT (6)..(6): Met, Leu, Ile, Val, Gln, or Phe
ARGAPXGAAAGWFDP

SEQ ID NO: 93 - protein, 9 aa, Artificial Sequence
  Description of Artificial Sequence Synthetic peptide
YTFTSYYMH

SEQ ID NO: 94 - protein, 17 aa, Artificial Sequence
  Description of Artificial Sequence Synthetic peptide
IINPSGGSTSYAQKFQG

SEQ ID NO: 95 - protein, 19 aa, Artificial Sequence
  Description of Artificial Sequence Synthetic peptide
ARGAPNYGDTTHDYYYMDV

SEQ ID NO: 96 - protein, 11 aa, Artificial Sequence
  Description of Artificial Sequence Synthetic peptide
RASQSVSSNLA

SEQ ID NO: 97 - protein, 7 aa, Artificial Sequence
  Description of Artificial Sequence Synthetic peptide
GASTRAT

SEQ ID NO: 98 - protein, 9 aa, Artificial Sequence
  Description of Artificial Sequence Synthetic peptide
QQYDDWPFT

SEQ ID NO: 99 - protein, 9 aa, Artificial Sequence
  Description of Artificial Sequence Synthetic peptide
YTFTGYYMH

SEQ ID NO: 100 - protein, 17 aa, Artificial Sequence
  Description of Artificial Sequence Synthetic peptide
WINPNSGGTNYAQKFQG

SEQ ID NO: 101 - protein, 17 aa, Artificial Sequence
  Description of Artificial Sequence Synthetic peptide
ARDTGEYYDTDDHGMDV

SEQ ID NO: 102 - protein, 11 aa, Artificial Sequence
  Description of Artificial Sequence Synthetic peptide
RASQSVSSNLA

SEQ ID NO: 103 - protein, 7 aa, Artificial Sequence
  Description of Artificial Sequence Synthetic peptide
GASTRAT

SEQ ID NO: 104 - protein, 9 aa, Artificial Sequence
  Description of Artificial Sequence Synthetic peptide
QQDDYWPPT

SEQ ID NO: 105 - protein, 9 aa, Artificial Sequence
  Description of Artificial Sequence Synthetic peptide
FTFSSYAMS

SEQ ID NO: 106 - protein, 17 aa, Artificial Sequence
  Description of Artificial Sequence Synthetic peptide
AISGSGGSTYYADSVKG

SEQ ID NO: 107 - protein, 14 aa, Artificial Sequence
  Description of Artificial Sequence Synthetic peptide
AKDGGYYDSGAGDY

SEQ ID NO: 108 - protein, 11 aa, Artificial Sequence
  Description of Artificial Sequence Synthetic peptide
RASQGIDSWLA

SEQ ID NO: 109 - protein, 7 aa, Artificial Sequence
  Description of Artificial Sequence Synthetic peptide
AASSLQS

SEQ ID NO: 110 - protein, 9 aa, Artificial Sequence
  Description of Artificial Sequence Synthetic peptide
QQGVSYPRT

SEQ ID NO: 111 - protein, 9 aa, Artificial Sequence
  Description of Artificial Sequence Synthetic peptide
FTFSSYSMN

SEQ ID NO: 112 - protein, 17 aa, Artificial Sequence
  Description of Artificial Sequence Synthetic peptide
SISSSSSYIYYADSVKG

SEQ ID NO: 113 - protein, 15 aa, Artificial Sequence
  Description of Artificial Sequence Synthetic peptide
ARGAPMGAAAGWFDP

SEQ ID NO: 114 - protein, 11 aa, Artificial Sequence
  Description of Artificial Sequence Synthetic peptide
RASQGISSWLA

SEQ ID NO: 115 - protein, 7 aa, Artificial Sequence
  Description of Artificial Sequence Synthetic peptide
AASSLQS

SEQ ID NO: 116 - protein, 9 aa, Artificial Sequence
  Description of Artificial Sequence Synthetic peptide
QQGVSFPRT

SEQ ID NO: 117 - protein, 9 aa, Artificial Sequence
  Description of Artificial Sequence Synthetic peptide
YTFTSYYMH

SEQ ID NO: 118 - protein, 17 aa, Artificial Sequence
  Description of Artificial Sequence Synthetic peptide
IINPSGGSTSYAQKFQG

SEQ ID NO: 119 - protein, 18 aa, Artificial Sequence
  Description of Artificial Sequence Synthetic peptide
AREGAGFAYGMDYYYMDV

SEQ ID NO: 120 - protein, 11 aa, Artificial Sequence
  Description of Artificial Sequence Synthetic peptide
RASQSVSSYLA

SEQ ID NO: 121 - protein, 7 aa, Artificial Sequence
  Description of Artificial Sequence Synthetic peptide
DASNRAT

SEQ ID NO: 122 - protein, 9 aa, Artificial Sequence
  Description of Artificial Sequence Synthetic peptide
QQSDNWPFT

SEQ ID NO: 123 - protein, 13 aa, Artificial Sequence
  Description of Artificial Sequence Synthetic peptide
  VARIANT (4)..(4): Met, Leu, Ile, Val, Gln, or Phe
GAPXGAAAGWFDP

SEQ ID NO: 124 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 125 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 126 - protein, 9 aa, Artificial Sequence
  Description of Artificial Sequence Synthetic peptide
GSFSGYYWS

SEQ ID NO: 127 - protein, 16 aa, Artificial Sequence
  Description of Artificial Sequence Synthetic peptide
EIDHSGSTNYNPSLKS

SEQ ID NO: 128 - protein, 11 aa, Artificial Sequence
  Description of Artificial Sequence Synthetic peptide
ARARGPWSFDP

SEQ ID NO: 129 - protein, 117 aa, Artificial Sequence; identical to SEQ ID NO: 124
  Description of Artificial Sequence Synthetic polypeptide

SEQ ID NO: 130 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 131 - protein, 117 aa, Artificial Sequence; identical to SEQ ID NO: 124
  Description of Artificial Sequence Synthetic polypeptide

SEQ ID NO: 132 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 133 - protein, 117 aa, Artificial Sequence; identical to SEQ ID NO: 124
  Description of Artificial Sequence Synthetic polypeptide

SEQ ID NO: 134 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 135 - protein, 117 aa, Artificial Sequence; identical to SEQ ID NO: 124
  Description of Artificial Sequence Synthetic polypeptide

SEQ ID NO: 136 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 137 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 138 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 139 - protein, 117 aa, Artificial Sequence; identical to SEQ ID NO: 124
  Description of Artificial Sequence Synthetic polypeptide

SEQ ID NO: 140 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 141 - protein, 117 aa, Artificial Sequence; identical to SEQ ID NO: 124
  Description of Artificial Sequence Synthetic polypeptide

SEQ ID NO: 142 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 143 - protein, 117 aa, Artificial Sequence; identical to SEQ ID NO: 124
  Description of Artificial Sequence Synthetic polypeptide

SEQ ID NO: 144 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 145 - protein, 117 aa, Artificial Sequence; identical to SEQ ID NO: 124
  Description of Artificial Sequence Synthetic polypeptide

SEQ ID NO: 146 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 147 - protein, 117 aa, Artificial Sequence; identical to SEQ ID NO: 124
  Description of Artificial Sequence Synthetic polypeptide

SEQ ID NO: 148 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 149 - protein, 117 aa, Artificial Sequence; identical to SEQ ID NO: 124
  Description of Artificial Sequence Synthetic polypeptide

SEQ ID NO: 150 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 151 - protein, 117 aa, Artificial Sequence; identical to SEQ ID NO: 124
  Description of Artificial Sequence Synthetic polypeptide

SEQ ID NO: 152 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 153 - protein, 117 aa, Artificial Sequence; identical to SEQ ID NO: 124
  Description of Artificial Sequence Synthetic polypeptide

SEQ ID NO: 154 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 155 - protein, 117 aa, Artificial Sequence; identical to SEQ ID NO: 124
  Description of Artificial Sequence Synthetic polypeptide

SEQ ID NO: 156 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 157 - protein, 117 aa, Artificial Sequence; identical to SEQ ID NO: 124
  Description of Artificial Sequence Synthetic polypeptide

SEQ ID NO: 158 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 159 - protein, 117 aa, Artificial Sequence; identical to SEQ ID NO: 124
  Description of Artificial Sequence Synthetic polypeptide

SEQ ID NO: 160 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 161 - protein, 117 aa, Artificial Sequence; identical to SEQ ID NO: 124
  Description of Artificial Sequence Synthetic polypeptide

SEQ ID NO: 162 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 163 - protein, 117 aa, Artificial Sequence; identical to SEQ ID NO: 124
  Description of Artificial Sequence Synthetic polypeptide

SEQ ID NO: 164 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 165 - protein, 117 aa, Artificial Sequence; identical to SEQ ID NO: 124
  Description of Artificial Sequence Synthetic polypeptide

SEQ ID NO: 166 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 167 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 168 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 169 - protein, 9 aa, Artificial Sequence
  Description of Artificial Sequence Synthetic peptide
GTFSSYAIS

SEQ ID NO: 170 - protein, 17 aa, Artificial Sequence
  Description of Artificial Sequence Synthetic peptide
GIIPIFGTANYAQKFQG

SEQ ID NO: 171 - protein, 18 aa, Artificial Sequence
  Description of Artificial Sequence Synthetic peptide
ARGDSSIRHAYYYYGMDV

SEQ ID NO: 172 - protein, 17 aa, Artificial Sequence
  Description of Artificial Sequence Synthetic peptide
KSSQSVLYSSNNKNYLA

SEQ ID NO: 173 - protein, 7 aa, Artificial Sequence
  Description of Artificial Sequence Synthetic peptide
WASTRES

SEQ ID NO: 174 - protein, 9 aa, Artificial Sequence
  Description of Artificial Sequence Synthetic peptide
QQYYSTPIT

SEQ ID NO: 175 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 176 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 177 - protein, 11 aa, Artificial Sequence
  Description of Artificial Sequence Synthetic peptide
GSISSSSYYWG

SEQ ID NO: 178 - protein, 16 aa, Artificial Sequence
  Description of Artificial Sequence Synthetic peptide
SIYYSGSTYYNPSLKS

SEQ ID NO: 179 - protein, 13 aa, Artificial Sequence
  Description of Artificial Sequence Synthetic peptide
ARGSDRFHPYFDY

SEQ ID NO: 180 - protein, 11 aa, Artificial Sequence
  Description of Artificial Sequence Synthetic peptide
RASQSVSRYLA

SEQ ID NO: 181 - protein, 5 aa, Artificial Sequence
  Description of Artificial Sequence Synthetic peptide
SYWMS

SEQ ID NO: 182 - protein, 10 aa, Artificial Sequence
  Description of Artificial Sequence Synthetic peptide
RPLNAGELDV

SEQ ID NO: 183 - protein, 5 aa, Artificial Sequence
  Description of Artificial Sequence Synthetic peptide
KYTMS

SEQ ID NO: 184 - protein, 19 aa, Artificial Sequence
  Description of Artificial Sequence Synthetic peptide
EGGPYYDSSGYFVYYGMDV

SEQ ID NO: 185 - protein, 6 aa, Artificial Sequence
  Description of Artificial Sequence Synthetic peptide
KASSLE

SEQ ID NO: 186 - protein, 20 aa, Artificial Sequence
  Description of Artificial Sequence Synthetic peptide
GGGGSGGGGSGGGGSGGGGS

SEQ ID NO: 187 - protein, 474 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 188 - protein, 246 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSS

SEQ ID NO: 189 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 190 - protein, 214 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 192 - protein, 5 aa, Artificial Sequence
  Description of Artificial Sequence Synthetic peptide
SYSMN

SEQ ID NO: 193 - protein, 13 aa, Artificial Sequence
  Description of Artificial Sequence Synthetic peptide
GAPMGAAAGWFDP

SEQ ID NO: 194 - protein, 15 aa, Artificial Sequence
  Description of Artificial Sequence Synthetic peptide
ARGAPIGAAAGWFDP

SEQ ID NO: 195 - protein, 13 aa, Artificial Sequence
  Description of Artificial Sequence Synthetic peptide
GAPIGAAAGWFDP

SEQ ID NO: 196 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 197 - protein, 482 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDDLPTFGCGTKVEIKGGGGSGGGGSGGGGSGGGGSEVQLLESGGGLVQPGGSLRLSCAASGFTFSKYTMSWVRQAPGKCLEWVSAIVGSGESTYFADSVKGRFTISRDNSKNTLYLQMNSLRAEDTAVYYCAREGGPYYDSSGYFVYYGMDVWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 198 - protein, 254 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDDLPTFGCGTKVEIKGGGGSGGGGSGGGGSGGGGSEVQLLESGGGLVQPGGSLRLSCAASGFTFSKYTMSWVRQAPGKCLEWVSAIVGSGESTYFADSVKGRFTISRDNSKNTLYLQMNSLRAEDTAVYYCAREGGPYYDSSGYFVYYGMDVWGQGTTVTVSS

SEQ ID NO: 199 - protein, 447 aa, Artificial Sequence
  Description of Artificial Sequence Synthetic polypeptide
EVQLVESGGGLVQPGGSLRLSCAASGFTFGSYWMSWVRQAPGKGLEWVATIKQDGSEKSYVDSVKGRFTISRDNAKNSLYLQMNSLRAEDTAVYYCARPLNAGELDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 200 - protein, 215 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 447 aa, Artificial Sequence
  Description of Artificial Sequence Synthetic polypeptide
EVQLVESGGGLVQPGGSLRLSCAASGFTFGSYWMSWVRQAPGKGLEWVATIKQDGSEKSYVDSVKGRFTISRDNAKNSLYLQMNSLRAEDTAVYYCARPLNAGELDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 446 aa, Artificial Sequence
  Description of Artificial Sequence Synthetic polypeptide
QVQLVQSGAEVKKPGSSVKVSCKASGGTFSDYAISWVRQAPGQGLEWMGRIIPILGVADYAQKFQGRVTITADKSTRTAYMELSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 213 aa, Artificial Sequence
  Description of Artificial Sequence Synthetic polypeptide
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 474 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSSASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 205 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 206 - protein, 254 aa, Artificial Sequence
  Description of Artificial Sequence Synthetic polypeptide
DIQMTQSPSTLSASVGDRVTITCRASQSISSWLAWYQQKPGKAPKLLIYDASSLESGVPSRFSGSGSGTEFTLTISSLQPDDFATYYCQQYESFPTFGCGTKVEIKGGGGSGGGGSGGGGSGGGGSEVQLVESGGGLVQPGGSLRLSCAASGFTFSSYGMSWVRQAPGKCLEWVANIKQDGSEKYYVDSVKGRFTISRDNAKNSLYLQMNSLRAEDTAVYYCAREGGPYYDSSGYFVYYGMDVWGQGTTVTVSS

SEQ ID NO: 207 - protein, 254 aa, Artificial Sequence
  Description of Artificial Sequence Synthetic polypeptide
EVQLVESGGGLVQPGGSLRLSCAASGFTFSSYGMSWVRQAPGKCLEWVANIKQDGSEKYYVDSVKGRFTISRDNAKNSLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYESFPTFGCGTKVEIK

SEQ ID NO: 208 - protein, 245 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQLESYPLTFGCGTKVEIKGGGGSGGGGSGGGGSGGGGSEVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARPLNAGELDVWGQGTMVTVSS

SEQ ID NO: 209 - protein, 245 aa, Artificial Sequence
  Description of Artificial Sequence Synthetic polypeptide
EVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQLESYPLTFGCGTKVEIK

SEQ ID NO: 210 - protein, 254 aa, Artificial Sequence
  Description of Artificial Sequence Synthetic polypeptide
EVQLLESGGGLVQPGGSLRLSCAASGFTFSKYTMSWVRQAPGKCLEWVSAIVGSGESTYFADSVKGRFTISRDNSKNTLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYDDLPTFGCGTKVEIK

SEQ ID NO: 211 - protein, 255 aa, Artificial Sequence
  Description of Artificial Sequence Synthetic polypeptide
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIKGGGGSGGGGSGGGGSGGGGSQVQLVQSGAEVKKPGASVKVSCKASGYTFSDYYMHWVRQAPGQCLEWMGMINPSWGSTSYAQKFQGRVTMTRDTSTSTVYMELSSLRSEDTAVYYCAREAADGFVGERYFDLWGRGTLVTVSS

SEQ ID NO: 212 - protein, 255 aa, Artificial Sequence
  Description of Artificial Sequence Synthetic polypeptide
QVQLVQSGAEVKKPGASVKVSCKASGYTFSDYYMHWVRQAPGQCLEWMGMINPSWGSTSYAQKFQGRVTMTRDTSTSTVYMELSSLRSEDTAVYYCAREAADGFVGERYFDLWGRGTLVTVSSGGGGSGGGGSGGGGSGGGGSDIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIK

SEQ ID NO: 213 - protein, 246 aa, Artificial Sequence
  Description of Artificial Sequence Synthetic polypeptide
EVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIK

SEQ ID NO: 214 - protein, 246 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFPSYWMSWVRQAPGKCLEWVATIKRDGSEKGYVDSVKGRFTISRDNAKNSLYLQMNSLRAEDTAVYYCARPLNAGELDVWGQGTMVTVSS

SEQ ID NO: 215 - protein, 246 aa, Artificial Sequence
  Description of Artificial Sequence Synthetic polypeptide
EVQLVESGGGLVQPGGSLRLSCAASGFTFPSYWMSWVRQAPGKCLEWVATIKRDGSEKGYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIK

SEQ ID NO: 216 - protein, 247 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIKGGGGSGGGGSGGGGSGGGGSQVQLVQSGAEVKKPGASVKVSCKASGYTFGTYYMHWVRQAPGQCLEWMGIINPSRGSTVYAQKFQGRVTMTRDTSTSTVYMELSSLRSEDTAVYYCARGAGYDDEDMDVWGKGTTVTVSS

SEQ ID NO: 217 - protein, 247 aa, Artificial Sequence
  Description of Artificial Sequence Synthetic polypeptide
QVQLVQSGAEVKKPGASVKVSCKASGYTFGTYYMHWVRQAPGQCLEWMGIINPSRGSTVYAQKFQGRVTMTRDTSTSTVYMELSSLRSEDTAVYYCARGAGYDDEDMDVWGKGTTVTVSSGGGGSGGGGSGGGGSGGGGSDIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIK

SEQ ID NO: 218 - protein, 254 aa, Artificial Sequence
  Description of Artificial Sequence Synthetic polypeptide
DIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGCGTKVEIKGGGGSGGGGSGGGGSGGGGSEVQLVESGGGLVKPGGSLRLSCAASGFTFSSYAMSWVRQAPGKCLEWVSSISSSSEGIYYADSVKGRFTISRDNAKNSLYLQMNSLRAEDTAVYYCAREGGPYYDSSGYFVYYGMDVWGQGTTVTVSS

SEQ ID NO: 219 - protein, 254 aa, Artificial Sequence
  Description of Artificial Sequence Synthetic polypeptide
EVQLVESGGGLVKPGGSLRLSCAASGFTFSSYAMSWVRQAPGKCLEWVSSISSSSEGIYYADSVKGRFTISRDNAKNSLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGCGTKVEIK

SEQ ID NO: 220 - protein, 250 aa, Artificial Sequence
  Description of Artificial Sequence Synthetic polypeptide
DIQMTQSPSSLSASVGDRVTITCRASQVIYSYLNWYQQKPGKAPKLLIYAASSLKSGVPSRFSGSGSGTDFTLTISSLQPEDFATYYCQQVYDTPLTFGCGTKVEIKGGGGSGGGGSGGGGSGGGGSEVQLVESGGGLVQPGGSLRLSCAASGFTFSSYWMSWVRQAPGKCLEWVANINTDGSEVYYVDSVKGRFTISRDNAKNSLYLQMNSLRAEDTAVYYCARDVGPGIAYQGHFDYWGQGTLVTVSS

SEQ ID NO: 221 - protein, 250 aa, Artificial Sequence
  Description of Artificial Sequence Synthetic polypeptide
EVQLVESGGGLVQPGGSLRLSCAASGFTFSSYWMSWVRQAPGKCLEWVANINTDGSEVYYVDSVKGRFTISRDNAKNSLYLQMNSLRAEDTAVYYCARDVGPGIAYQGHFDYWGQGTLVTVSSGGGGSGGGGSGGGGSGGGGSDIQMTQSPSSLSASVGDRVTITCRASQVIYSYLNWYQQKPGKAPKLLIYAASSLKSGVPSRFSGSGSGTDFTLTISSLQPEDFATYYCQQVYDTPLTFGCGTKVEIK

SEQ ID NO: 222 - protein, 246 aa, Artificial Sequence
  Description of Artificial Sequence Synthetic polypeptide
EIVLTQSPATLSLSPGERATLSCRASHSVYSYLAWYQQKPGQAPRLLIYDASNRATGIPARFSGSGSGTDFTLTISSLEPEDFAVYYCQQYDNLPTFGCGTKVEIKGGGGSGGGGSGGGGSGGGGSQLQLQESGPGLVKPSETLSLTCTVSGGSISSTDYYWGWIRQPPGKCLEWIGSIGYSGTYYNPSLKSRVTISVDTSKNQFSLKLSSVTAADTAVYYCARETAHDVHGMDVWGQGTTVTVSS

SEQ ID NO: 223 - protein, 246 aa, Artificial Sequence
  Description of Artificial Sequence Synthetic polypeptide
QLQLQESGPGLVKPSETLSLTCTVSGGSISSTDYYWGWIRQPPGKCLEWIGSIGYSGTYYNPSLKSRVTISVDTSKNQFSLKLSSVTAADTAVYYCARETAHDVHGMDVWGQGTTVTVSSGGGGSGGGGSGGGGSGGGGSEIVLTQSPATLSLSPGERATLSCRASHSVYSYLAWYQQKPGQAPRLLIYDASNRATGIPARFSGSGSGTDFTLTISSLEPEDFAVYYCQQYDNLPTFGCGTKVEIK

SEQ ID NO: 224 - protein, 482 aa, Artificial Sequence
  Description of Artificial Sequence Synthetic polypeptide
DIQMTQSPSTLSASVGDRVTITCRASQSISSWLAWYQQKPGKAPKLLIYDASSLESGVPSRFSGSGSGTEFTLTISSLQPDDFATYYCQQYESFPTFGCGTKVEIKGGGGSGGGGSGGGGSGGGGSEVQLVESGGGLVQPGGSLRLSCAASGFTFSSYGMSWVRQAPGKCLEWVANIKQDGSEKYYVDSVKGRFTISRDNAKNSLYLQMNSLRAEDTAVYYCAREGGPYYDSSGYFVYYGMDVWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 225 - protein, 482 aa, Artificial Sequence
  Description of Artificial Sequence Synthetic polypeptide
EVQLVESGGGLVQPGGSLRLSCAASGFTFSSYGMSWVRQAPGKCLEWVANIKQDGSEKYYVDSVKGRFTISRDNAKNSLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASQSISSWLAWYQQKPGKAPKLLIYDASSLESGVPSRFSGSGSGTEFTLTISSLQPDDFATYYCQQYESFPTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 226 - protein, 473 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQLESYPLTFGCGTKVEIKGGGGSGGGGSGGGGSGGGGSEVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARPLNAGELDVWGQGTMVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 227 - protein, 473 aa, Artificial Sequence
  Description of Artificial Sequence Synthetic polypeptide
EVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQLESYPLTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 228 - protein, 482 aa, Artificial Sequence
  Description of Artificial Sequence Synthetic polypeptide
EVQLLESGGGLVQPGGSLRLSCAASGFTFSKYTMSWVRQAPGKCLEWVSAIVGSGESTYFADSVKGRFTISRDNSKNTLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYDDLPTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 229 - protein, 483 aa, Artificial Sequence
  Description of Artificial Sequence Synthetic polypeptide
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIKGGGGSGGGGSGGGGSGGGGSQVQLVQSGAEVKKPGASVKVSCKASGYTFSDYYMHWVRQAPGQCLEWMGMINPSWGSTSYAQKFQGRVTMTRDTSTSTVYMELSSLRSEDTAVYYCAREAADGFVGERYFDLWGRGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 230 - protein, 483 aa, Artificial Sequence
  Description of Artificial Sequence Synthetic polypeptide
QVQLVQSGAEVKKPGASVKVSCKASGYTFSDYYMHWVRQAPGQCLEWMGMINPSWGSTSYAQKFQGRVTMTRDTSTSTVYMELSSLRSEDTAVYYCAREAADGFVGERYFDLWGRGTLVTVSSGGGGSGGGGSGGGGSGGGGSDIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 231 - protein, 474 aa, Artificial Sequence
  Description of Artificial Sequence Synthetic polypeptide
EVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 232 - protein, 474 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFPSYWMSWVRQAPGKCLEWVATIKRDGSEKGYVDSVKGRFTISRDNAKNSLYLQMNSLRAEDTAVYYCARPLNAGELDVWGQGTMVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 233 - protein, 474 aa, Artificial Sequence
  Description of Artificial Sequence Synthetic polypeptide
EVQLVESGGGLVQPGGSLRLSCAASGFTFPSYWMSWVRQAPGKCLEWVATIKRDGSEKGYVDSVKGRFTISRDNAKNSLYLQMNSLRAEDTAVYYCARPLNAGELDVWGQGTMVTVSSGGGGSGGGGSGGGGSGGGGSDIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 234 - protein, 475 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIKGGGGSGGGGSGGGGSGGGGSQVQLVQSGAEVKKPGASVKVSCKASGYTFGTYYMHWVRQAPGQCLEWMGIINPSRGSTVYAQKFQGRVTMTRDTSTSTVYMELSSLRSEDTAVYYCARGAGYDDEDMDVWGKGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 235 - protein, 475 aa, Artificial Sequence
  Description of Artificial Sequence Synthetic polypeptide
QVQLVQSGAEVKKPGASVKVSCKASGYTFGTYYMHWVRQAPGQCLEWMGIINPSRGSTVYAQKFQGRVTMTRDTSTSTVYMELSSLRSEDTAVYYCARGAGYDDEDMDVWGKGTTVTVSSGGGGSGGGGSGGGGSGGGGSDIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 236 - protein, 482 aa, Artificial Sequence
  Description of Artificial Sequence Synthetic polypeptide
DIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGCGTKVEIKGGGGSGGGGSGGGGSGGGGSEVQLVESGGGLVKPGGSLRLSCAASGFTFSSYAMSWVRQAPGKCLEWVSSISSSSEGIYYADSVKGRFTISRDNAKNSLYLQMNSLRAEDTAVYYCAREGGPYYDSSGYFVYYGMDVWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 237 - protein, 482 aa, Artificial Sequence
  Description of Artificial Sequence Synthetic polypeptide
EVQLVESGGGLVKPGGSLRLSCAASGFTFSSYAMSWVRQAPGKCLEWVSSISSSSEGIYYADSVKGRFTISRDNAKNSLYLQMNSLRAEDTAVYYCAREGGPYYDSSGYFVYYGMDVWGQGTTVTVSSGGGGSGGGGSGGGGSGGGGSDIQMTQSPSTLSASVGDRVTITCRASNSISSWLAWYQQKPGKAPKLLIYEASSTKSGVPSRFSGSGSGTEFTLTISSLQPDDFATYYCQQYDDLPTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 238 - protein, 478 aa, Artificial Sequence
  Description of Artificial Sequence Synthetic polypeptide
DIQMTQSPSSLSASVGDRVTITCRASQVIYSYLNWYQQKPGKAPKLLIYAASSLKSGVPSRFSGSGSGTDFTLTISSLQPEDFATYYCQQVYDTPLTFGCGTKVEIKGGGGSGGGGSGGGGSGGGGSEVQLVESGGGLVQPGGSLRLSCAASGFTFSSYWMSWVRQAPGKCLEWVANINTDGSEVYYVDSVKGRFTISRDNAKNSLYLQMNSLRAEDTAVYYCARDVGPGIAYQGHFDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 239 - protein, 478 aa, Artificial Sequence
  Description of Artificial Sequence Synthetic polypeptide
EVQLVESGGGLVQPGGSLRLSCAASGFTFSSYWMSWVRQAPGKCLEWVANINTDGSEVYYVDSVKGRFTISRDNAKNSLYLQMNSLRAEDTAVYYCARDVGPGIAYQGHFDYWGQGTLVTVSSGGGGSGGGGSGGGGSGGGGSDIQMTQSPSSLSASVGDRVTITCRASQVIYSYLNWYQQKPGKAPKLLIYAASSLKSGVPSRFSGSGSGTDFTLTISSLQPEDFATYYCQQVYDTPLTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 240 - protein, 474 aa, Artificial Sequence
  Description of Artificial Sequence Synthetic polypeptide
EIVLTQSPATLSLSPGERATLSCRASHSVYSYLAWYQQKPGQAPRLLIYDASNRATGIPARFSGSGSGTDFTLTISSLEPEDFAVYYCQQYDNLPTFGCGTKVEIKGGGGSGGGGSGGGGSGGGGSQLQLQESGPGLVKPSETLSLTCTVSGGSISSTDYYWGWIRQPPGKCLEWIGSIGYSGTYYNPSLKSRVTISVDTSKNQFSLKLSSVTAADTAVYYCARETAHDVHGMDVWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 241 - protein, 474 aa, Artificial Sequence
  Description of Artificial Sequence Synthetic polypeptide
QLQLQESGPGLVKPSETLSLTCTVSGGSISSTDYYWGWIRQPPGKCLEWIGSIGYSGTYYNPSLKSRVTISVDTSKNQFSLKLSSVTAADTAVYYCARETAHDVHGMDVWGQGTTVTVSSGGGGSGGGGSGGGGSGGGGSEIVLTQSPATLSLSPGERATLSCRASHSVYSYLAWYQQKPGQAPRLLIYDASNRATGIPARFSGSGSGTDFTLTISSLEPEDFAVYYCQQYDNLPTFGCGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 242 - protein, 575 aa, Artificial Sequence
  Description of Artificial Sequence Synthetic polypeptide
DIQMTQSPSTLSASVGDRVTITCRASQSISSWLAWYQQKPGKAPKLLIYEASSLESGVPSRFSGSGSGTEFTLTISSLQPDDFATYYCQQSQSYPPITFGCGTKVEIKGGGGSGGGGSGGGGSGGGGSEVQLVESGGGLVQPGGSLRLSCAASGFTFGSYWMSWVRQAPGKCLEWVATIKQDGSEKSYVDSVKGRFTISRDNAKNSLYLQMNSLRAEDTAVYYCARPLNAGELDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 243 - protein, 583 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDDLPTFGCGTKVEIKGGGGSGGGGSGGGGSGGGGSEVQLLESGGGLVQPGGSLRLSCAASGFTFSKYTMSWVRQAPGKCLEWVSAIVGSGESTYFADSVKGRFTISRDNSKNTLYLQMNSLRAEDTAVYYCAREGGPYYDSSGYFVYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 244 - protein, 347 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSL

SEQ ID NO: 245 - protein, 347 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSL

SEQ ID NO: 246 - DNA, 762 bp, Artificial Sequence
  Description of Artificial Sequence Synthetic polynucleotide
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgacgccagcagcctggagagcggcgtgcccagcaggttcagcggcagcggcagcggcaccgagttcaccctgaccatcagcagcctgcagcccgacgacttcgccacctactactgccagcagtacgagagcttccccaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctacggcatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagc

SEQ ID NO: 247 - DNA, 762 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctacggcatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgacgccagcagcctggagagcggcgtgcccagcaggttcagcggcagcggcagcggcaccgagttcaccctgaccatcagcagcctgcagcccgacgacttcgccacctactactgccagcagtacgagagcttccccaccttcggctgcggcaccaaggtggagatcaag

SEQ ID NO: 248 - DNA, 735 bp, Artificial Sequence
  Description of Artificial Sequence Synthetic polynucleotide
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagctggagagctaccccctgaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagc

SEQ ID NO: 249 - DNA, 735 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagctggagagctaccccctgaccttcggctgcggcaccaaggtggagatcaag

SEQ ID NO: 250 - DNA, 762 bp, Artificial Sequence
  Description of Artificial Sequence Synthetic polynucleotide
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacaaggccagcagcctggagagcggcgtgcccagcaggttcagcggcagcggcagcggcaccgagttcaccctgaccatcagcagcctgcagcccgacgacttcgccacctactactgccagcagtacgacgacctgcccaccttcggctgcggcaccaaggtggagatcaagggcggcggcggcagcggcggcggcggcagcggcggcggcggcagcggcggcggcggcagcgaggtgcagctgctggagagcggcggcggcctggtgcagcccggcggcagcctgaggctgagctgcgccgccagcggcttcaccttcagcaagtacaccatgagctgggtgaggcaggcccccggcaagtgcctggagtgggtgagcgccatcgtgggcagcggcgagagcacctacttcgccgacagcgtgaagggcaggttcaccatcagcagggacaacagcaagaacaccctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagc

SEQ ID NO: 251 - DNA, 762 bp, Artificial Sequence
  Description of Artificial Sequence Synthetic polynucleotide
gaggtgcagctgctggagagcggcggcggcctggtgcagcccggcggcagcctgaggctgagctgcgccgccagcggcttcaccttcagcaagtacaccatgagctgggtgaggcaggcccccggcaagtgcctggagtgggtgagcgccatcgtgggcagcggcgagagcacctacttcgccgacagcgtgaagggcaggttcaccatcagcagggacaacagcaagaacaccctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacaaggccagcagcctggagagcggcgtgcccagcaggttcagcggcagcggcagcggcaccgagttcaccctgaccatcagcagcctgcagcccgacgacttcgccacctactactgccagcagtacgacgacctgcccaccttcggctgcggcaccaaggtggagatcaag

SEQ ID NO: 252 - DNA, 765 bp, Artificial Sequence
  Description of Artificial Sequence Synthetic polynucleotide
gacatcgtgatgacccagagccccctgagcctgcccgtgacccccggcgagcccgccagcatcagctgcaggagcagccagagcctgctgtacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggacgtggccctgcccatcaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcagcgactactacatgcactgggtgaggcaggcccccggccagtgcctggagtggatgggcatgatcaaccccagctggggcagcaccagctacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagc

SEQ ID NO: 253 - DNA, 765 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcagcgactactacatgcactgggtgaggcaggcccccggccagtgcctggagtggatgggcatgatcaaccccagctggggcagcaccagctacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagcggcggcggcggcagcggcggcggcggcagcggcggcggcggcagcggcggcggcggcagcgacatcgtgatgacccagagccccctgagcctgcccgtgacccccggcgagcccgccagcatcagctgcaggagcagccagagcctgctgtacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggacgtggccctgcccatcaccttcggctgcggcaccaaggtggagatcaag

SEQ ID NO: 254 - DNA, 738 bp, Artificial Sequence
  Description of Artificial Sequence Synthetic polynucleotide
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagagccagagctacccccccatcaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcggcagctactggatgagctgggtgaggcaggcccccggcaagtgcctggagtgggtggccaccatcaagcaggacggcagcgagaagagctacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagc

SEQ ID NO: 255 - DNA, 738 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcggcagctactggatgagctgggtgaggcaggcccccggcaagtgcctggagtgggtggccaccatcaagcaggacggcagcgagaagagctacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagagccagagctacccccccatcaccttcggctgcggcaccaaggtggagatcaag

SEQ ID NO: 256 - DNA, 738 bp, Artificial Sequence
  Description of Artificial Sequence Synthetic polynucleotide
gacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagagccagagctacccccccatcaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttccccagctactggatgagctgggtgaggcaggcccccggcaagtgcctggagtgggtggccaccatcaagagggacggcagcgagaagggctacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagc

SEQ ID NO: 257 - DNA, 738 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttccccagctactggatgagctgggtgaggcaggcccccggcaagtgcctggagtgggtggccaccatcaagagggacggcagcgagaagggctacgtggacagcgtgaagggcaggttcaccatcagcagggacaacgccaagaacagcctgtacctgcagatgaacagcctgagggccgaggacaccgccgtgtactactgcgccaggcccctgaacgccggcgagctggacgtgtggggccagggcaccatggtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagccagagcatcagcagctggctggcctggtaccagcagaagcccggcaaggcccccaagctgctgatctacgaggccagcagcctggagagcggcgtgcccagcaggttcagcggcagcggcagcggcaccgagttcaccctgaccatcagcagcctgcagcccgacgacttcgccacctactactgccagcagagccagagctacccccccatcaccttcggctgcggcaccaaggtggagatcaag

SEQ ID NO: 258 - DNA, 741 bp, Artificial Sequence
  Description of Artificial Sequence Synthetic polynucleotide
gacatccagatgacccagagccccagcagcgtgagcgccagcgtgggcgacagggtgaccatcacctgcagggccagccagggcatcgacagctggctggcctggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggcccacagctaccccctgaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcggcacctactacatgcactgggtgaggcaggcccccggccagtgcctggagtggatgggcatcatcaaccccagcaggggcagcaccgtgtacgcccagaagttccagggcagggtgaccatgaccagggacaccagcaccagcaccgtgtacatggagctgagcagcctgaggagcgaggacaccgccgtgtactactgcgccaggggcgccggctacgacgacgaggacatggacgtgtggggcaagggcaccaccgtgaccgtgagcagc

SEQ ID NO: 259 - DNA, 741 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcggcacctactacatgcactgggtgaggcaggcccccggccagtgcctggagtggatgggcatcatcaaccccagcaggggcagcaccgtgtacgcccagaagttccagggcagggtgaccatgaccagggacaccagcaccagcaccgtgtacatggagctgagcagcctgaggagcgaggacaccgccgtgtactactgcgccaggggcgccggctacgacgacgaggacatggacgtgtggggcaagggcaccaccgtgaccgtgagcagcggcggcggcggcagcggcggcggcggcagcggcggcggcggcagcggcggcggcggcagcgacatccagatgacccagagccccagcagcgtgagcgccagcgtgggcgacagggtgaccatcacctgcagggccagccagggcatcgacagctggctggcctggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggcccacagctaccccctgaccttcggctgcggcaccaaggtggagatcaag

SEQ ID NO: 260 - DNA, 762 bp, Artificial Sequence
  Description of Artificial Sequence Synthetic polynucleotide
gacatccagatgacccagagccccagcaccctgagcgccagcgtgggcgacagggtgaccatcacctgcagggccagcaacagcatcagcagctggctggcctggtaccagcagaagcccggcaaggcccccaagctgctgatctacgaggccagcagcaccaagagcggcgtgcccagcaggttcagcggcagcggcagcggcaccgagttcaccctgaccatcagcagcctgcagcccgacgacttcgccacctactactgccagcagtacgacgacctgcccaccttcggctgcggcaccaaggtggagatcaagggcggcggcggcagcggcggcggcggcagcggcggcggcggcagcggcggcggcggcagcgaggtgcagctggtggagagcggcggcggcctggtgaagcccggcggcagcctgaggctgagctgcgccgccagcggcttcaccttcagcagctacgccatgagctgggtgaggcaggcccccggcaagtgcctggagtgggtgagcagcatcagcagcagcagcgagggcatctactacgccgacagcgtgaagggcaggttcaccatcagcagggacaacgccaagaacagcctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagc

SEQ ID NO: 261 - DNA, 762 bp, Artificial Sequence
  Description of Artificial Sequence Synthetic polynucleotide
gaggtgcagctggtggagagcggcggcggcctggtgaagcccggcggcagcctgaggctgagctgcgccgccagcggcttcaccttcagcagctacgccatgagctgggtgaggcaggcccccggcaagtgcctggagtgggtgagcagcatcagcagcagcagcgagggcatctactacgccgacagcgtgaagggcaggttcaccatcagcagggacaacgccaagaacagcctgtacctgcagatgaacagcctgagggccgaggacaccgccgtgtactactgcgccagggagggcggcccctactacgacagcagcggctacttcgtgtactacggcatggacgtgtggggccagggcaccaccgtgaccgtgagcagcggcggcggcggcagcggcggcggcggcagcggcggcggcggcagcggcggcggcggcagcgacatccagatgacccagagccccagcaccctgagcgccagcgtgggcgacagggtgaccatcacctgcagggccagcaacagcatcagcagctggctggcctggtaccagcagaagcccggcaaggcccccaagctgctgatctacgaggccagcagcaccaagagcggcgtgcccagcaggttcagcggcagcggcagcggcaccgagttcaccctgaccatcagcagcctgcagcccgacgacttcgccacctactactgccagcagtacgacgacctgcccaccttcggctgcggcaccaaggtggagatcaag

SEQ ID NO: 262 - DNA, 750 bp, Artificial Sequence
  Description of Artificial Sequence Synthetic polynucleotide
gacatccagatgacccagagccccagcagcctgagcgccagcgtgggcgacagggtgaccatcacctgcagggccagccaggtgatctacagctacctgaactggtaccagcagaagcccggcaaggcccccaagctgctgatctacgccgccagcagcctgaagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaacaccgacggcagcgaggtgtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagc

SEQ ID NO: 263 - DNA, 750 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaacaccgacggcagcgaggtgtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccaggtgatctacagctacctgaactggtaccagcagaagcccggcaaggcccccaagctgctgatctacgccgccagcagcctgaagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaag

SEQ ID NO: 264 - DNA, 738 bp, Artificial Sequence
  Description of Artificial Sequence Synthetic polynucleotide
gagatcgtgctgacccagagccccgccaccctgagcctgagccccggcgagagggccaccctgagctgcagggccagccacagcgtgtacagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagtacgacaacctgcccaccttcggctgcggcaccaaggtggagatcaagggcggcggcggcagcggcggcggcggcagcggcggcggcggcagcggcggcggcggcagccagctgcagctgcaggagagcggccccggcctggtgaagcccagcgagaccctgagcctgacctgcaccgtgagcggcggcagcatcagcagcaccgactactactggggctggatcaggcagccccccggcaagtgcctggagtggatcggcagcatcggctacagcggcacctactacaaccccagcctgaagagcagggtgaccatcagcgtggacaccagcaagaaccagttcagcctgaagctgagcagcgtgaccgccgccgacaccgccgtgtactactgcgccagggagaccgcccacgacgtgcacggcatggacgtgtggggccagggcaccaccgtgaccgtgagcagc

SEQ ID NO: 265 - DNA, 738 bp, Artificial Sequence
  Description of Artificial Sequence Synthetic polynucleotide
cagctgcagctgcaggagagcggccccggcctggtgaagcccagcgagaccctgagcctgacctgcaccgtgagcggcggcagcatcagcagcaccgactactactggggctggatcaggcagccccccggcaagtgcctggagtggatcggcagcatcggctacagcggcacctactacaaccccagcctgaagagcagggtgaccatcagcgtggacaccagcaagaaccagttcagcctgaagctgagcagcgtgaccgccgccgacaccgccgtgtactactgcgccagggagaccgcccacgacgtgcacggcatggacgtgtggggccagggcaccaccgtgaccgtgagcagcggcggcggcggcagcggcggcggcggcagcggcggcggcggcagcggcggcggcggcagcgagatcgtgctgacccagagccccgccaccctgagcctgagccccggcgagagggccaccctgagctgcagggccagccacagcgtgtacagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagtacgacaacctgcccaccttcggctgcggcaccaaggtggagatcaag

SEQ ID NO: 266 - protein, 127 aa, Artificial Sequence
  Description of Artificial Sequence Synthetic polypeptide
EVQLLESGGGLVQPGGSLRLSCAASGFTFSSYAMSWVRQAPGKGLEWVSAISASGGSTYYADSVKGRFTISRDNSKNTLYLQMNSLRAEDTAVYYCARPRAYYDSSGFKVNYGMDVWGQGTTVTVSS

SEQ ID NO: 267 - protein, 108 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQASSSPPTFGGGTKVEIK

SEQ ID NO: 268 - protein, 123 aa, Artificial Sequence
  Description of Artificial Sequence Synthetic polypeptide
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCAREGHSSSYYDHAFDIWGQGTMVTVSS

SEQ ID NO: 269 - protein, 108 aa, Artificial Sequence
  Description of Artificial Sequence Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQQHSSAPPTFGGGTKVEIK

SEQ ID NO: 270 - protein, 123 aa, Artificial Sequence
  Description of Artificial Sequence Synthetic polypeptide
QVQLQESGPGLVKPSETLSLTCTVSGGSISSYYWSWIRQPPGKGLEWIGSIYYSGSTNYNPSLKSRVTISVDTSKNQFSLKLSSVTAADTAVYYCARVGGVYSTIETYGMDVWGQGTTVTVSS

SEQ ID NO: 271 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYTVYPPTFGGGTKVEIK

SEQ ID NO: 272 - protein, 119 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARQGIHGLRYFDLWGRGTLVTVSS

SEQ ID NO: 273 - protein, 107 aa, Artificial Sequence
  Description of Artificial Sequence Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQDHNFPYTFGGGTKVEIK

SEQ ID NO: 274 - protein, 121 aa, Artificial Sequence
  Description of Artificial Sequence Synthetic polypeptide
EVQLVESGGGLVQPGGSLRLSCAASGFTFSSYWMSWVRQAPGKGLEWVANINQDGSEKYYVDSVKGRFTISRDNAKNSLYLQMNSLRAEDTAVYYCAREANYYGNVGDDYWGQGTLVTVSS

SEQ ID NO: 275 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQQYVTPITFGGGTKVEIK

SEQ ID NO: 276 - protein, 121 aa, Artificial Sequence
  Description of Artificial Sequence Synthetic polypeptide
EVQLVESGGGLVQPGGSLRLSCAASGFTFSSYWMSWVRQAPGKGLEWVANINQDGSEKYYVDSVKGRFTISRDNAKNSLYLQMNSLRAEDTAVYYCAREGGDSWYHAFDIWGQGTMVTVSS

SEQ ID NO: 277 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASNLQSGVPSRFSGSGSGTDFTLTISSLQPEDFATYYCQQKLSLPLTFGGGTKVEIK

SEQ ID NO: 278 - protein, 123 aa, Artificial Sequence
  Description of Artificial Sequence Synthetic polypeptide
QVQLQESGPGLVKPSQTLSLTCTVSGGSISSGGYYWSWIRQHPGKGLEWIGSIYYSGSTYYNPSLKSRVTISVDTSKNQFSLKLSSVTAADTAVYYCARDRLDYSYNYGMDVWGQGTTVTVSS

SEQ ID NO: 279 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVYSAPFTFGGGTKVEIK

SEQ ID NO: 280 - protein, 121 aa, Artificial Sequence
  Description of Artificial Sequence Synthetic polypeptide
QVQLQESGPGLVKPSETLSLTCAVSGYSISSGYYWGWIRQPPGKGLEWIGSIYHSGSTNYNPSLKSRVTISVDTSKNQFSLKLSSVTAADTAVYYCARLPPWFGFSYFDLWGRGTLVTVSS

SEQ ID NO: 281 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQVDNYPPTFGGGTKVEIK

SEQ ID NO: 282 - protein, 123 aa, Artificial Sequence
  Description of Artificial Sequence Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDVGPGIAYQGHFDYWGQGTLVTVSS

SEQ ID NO: 283 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQVYDTPLTFGGGTKVEIK

SEQ ID NO: 284 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETAHDVHGMDVWGQGTTVTVSS

SEQ ID NO: 285 - protein, 106 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQYDNLPTFGGGTKVEIK

SEQ ID NO: 286 - protein, 123 aa, Artificial Sequence
  Description of Artificial Sequence Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREVGYGWYTKIAFDIWGQGTMVTVSS

SEQ ID NO: 287 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQSSNHPSTFGGGTKVEIK

SEQ ID NO: 288 - protein, 123 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTTYAQKFQGRVTMTRDTSTSTVYMELSSLRSEDTAVYYCAREAADGFVGERYFDLWGRGTLVTVSS

SEQ ID NO: 289 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGVPLTFGGGTKVEIK

SEQ ID NO: 290 - protein, 123 aa, Artificial Sequence
  Description of Artificial Sequence Synthetic polypeptide
QVQLVQSGAEVKKPGASVKVSCKASGYTFSGYYMHWVRQAPGQGLEWMGMINPYGGSTRYAQKFQGRVTMTRDTSTSTVYMELSSLRSEDTAVYYCAREAADGFVGERYFDLWGRGTLVTVSS

SEQ ID NO: 291 - protein, 112 aa, Artificial Sequence; identical to SEQ ID NO: 8
  Description of Artificial Sequence Synthetic polypeptide

SEQ ID NO: 292 - protein, 120 aa, Artificial Sequence
  Description of Artificial Sequence Synthetic polypeptide
QVQLVQSGAEVKKPGASVKVSCKASGYTFEIYYMHWVRQAPGQGLEWMGIINPSSGSTVYAQKFQGRVTMTRDTSTSTVYMELSSLRSEDTAVYYCARGAGYDDEDMDVWGKGTTVTVSS

SEQ ID NO: 293 - protein, 107 aa, Artificial Sequence; identical to SEQ ID NO: 14
  Description of Artificial Sequence Synthetic polypeptide

SEQ ID NO: 294 - protein, 121 aa, Artificial Sequence
  Description of Artificial Sequence Synthetic polypeptide
EVQLVESGGGLVQPGGSLRLSCAASGFTFGGYWMSWVRQAPGKGLEWVANINQDGSEEYYVDSVKGRFTISRDNAKNSLYLQMNSLRAEDTAVYYCAREANYYGNVGDDYWGQGTLVTVSS

SEQ ID NO: 295 - protein, 107 aa, Artificial Sequence
  Description of Artificial Sequence Synthetic polypeptide
DIQMTQSPSSLSASVGDRVTITCRASQSIYNYLNWYQQKPGKAPKLLIYAASNLHSGVPSRFSGSGSGTDFTLTISSLQPEDFATYYCQQAFHVPITFGGGTKVEIK

SEQ ID NO: 296 - protein, 121 aa, Artificial Sequence
  Description of Artificial Sequence Synthetic polypeptide
EVQLVESGGGLVQPGGSLRLSCAASGFTFPGYWMSWVRQAPGKGLEWVANINQDGSEVYYVDSVKGRFTISRDNAKNSLYLQMNSLRAEDTAVYYCAREANYYGNVGDDYWGQGTLVTVSS

SEQ ID NO: 297 - protein, 107 aa, Artificial Sequence
  Description of Artificial Sequence Synthetic polypeptide
DIQMTQSPSSLSASVGDRVTITCRASQSIYNYLNWYQQKPGKAPKLLIYAASSTQSGVPSRFSGSGSGTDFTLTISSLQPEDFATYYCQQAFHVPITFGGGTKVEIK

SEQ ID NO: 298 - protein, 123 aa, Artificial Sequence
  Description of Artificial Sequence Synthetic polypeptide
EVQLVESGGGLVQPGGSLRLSCAASGFTFSSYWMSWVRQAPGKGLEWVANINQDGSEVYYVDSVKGRFTISRDNAKNSLYLQMNSLRAEDTAVYYCARDVGPGIAYQGHFDYWGQGTLVTVSS

SEQ ID NO: 299 - protein, 107 aa, Artificial Sequence
  Description of Artificial Sequence Synthetic polypeptide
DIQMTQSPSSLSASVGDRVTITCRASQSIYYYLNWYQQKPGKAPKLLIYAASSRQSGVPSRFSGSGSGTDFTLTISSLQPEDFATYYCQQVYDTPLTFGGGTKVEIK

SEQ ID NO: 300 - protein, 121 aa, Artificial Sequence
  Description of Artificial Sequence Synthetic polypeptide
QVQLVQSGAEVKKPGASVKVSCKASGYTFSNYYMHWVRQAPGQGLEWMGWINPFSGGTRYAQKFQGRVTMTRDTSTSTVYMELSSLRSEDTAVYYCARDVGSSAYYYMDVWGKGTTVTVSS

SEQ ID NO: 301 - protein, 107 aa, Artificial Sequence
  Description of Artificial Sequence Synthetic polypeptide
DIQMTQSPSSVSASVGDRVTITCEASKGISSWLAWYQQKPGKAPKLLIYAASDLQSGVPSRFSGSGSGTDFTLTISSLQPEDFATYYCQQAFLFPPTFGGGTKVEIK

SEQ ID NO: 302 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGSIYPGDSDTRYSPSFQGQVTISADKSISTAYLQWSSLKASDTAMYYCARELAYGDYKGGVDYWGQGTLVTVSS

SEQ ID NO: 303 - protein, 108 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQLDSPPPTFGGGTKVEIK

SEQ ID NO: 304 - protein, 5 aa, Artificial Sequence
  Description of Artificial Sequence Synthetic peptide
SYAMS

SEQ ID NO: 305 - protein, 17 aa, Artificial Sequence
  Description of Artificial Sequence Synthetic peptide
AISASGGSTYYADSVKG

SEQ ID NO: 306 - protein, 18 aa, Artificial Sequence
  Description of Artificial Sequence Synthetic peptide
PRAYYDSSGFKVNYGMDV

SEQ ID NO: 307 - protein, 12 aa, Artificial Sequence
  Description of Artificial Sequence Synthetic peptide
RASQSVSSSFLA

SEQ ID NO: 308 - protein, 7 aa, Artificial Sequence
  Description of Artificial Sequence Synthetic peptide
GASSRAT

SEQ ID NO: 309 - protein, 9 aa, Artificial Sequence
  Description of Artificial Sequence Synthetic peptide
QQASSSPPT

SEQ ID NO: 310 - protein, 5 aa, Artificial Sequence
  Description of Artificial Sequence Synthetic peptide
SYAMS

SEQ ID NO: 311 - protein, 17 aa, Artificial Sequence
  Description of Artificial Sequence Synthetic peptide
GISGSGGSTYYADSVKG

SEQ ID NO: 312 - protein, 14 aa, Artificial Sequence
  Description of Artificial Sequence Synthetic peptide
EGHSSSYYDHAFDI

SEQ ID NO: 313 - protein, 12 aa, Artificial Sequence
  Description of Artificial Sequence Synthetic peptide
RASQSVSSDYLA

SEQ ID NO: 314 - protein, 7 aa, Artificial Sequence
  Description of Artificial Sequence Synthetic peptide
GASSRAT

SEQ ID NO: 315 - protein, 9 aa, Artificial Sequence
  Description of Artificial Sequence Synthetic peptide
QQHSSAPPT

SEQ ID NO: 316 - protein, 5 aa, Artificial Sequence
  Description of Artificial Sequence Synthetic peptide
SYYWS

SEQ ID NO: 317 - protein, 16 aa, Artificial Sequence
  Description of Artificial Sequence Synthetic peptide
SIYYSGSTNYNPSLKS

SEQ ID NO: 318 - protein, 15 aa, Artificial Sequence
  Description of Artificial Sequence Synthetic peptide
VGGVYSTIETYGMDV

SEQ ID NO: 319 - protein, 11 aa, Artificial Sequence
  Description of Artificial Sequence Synthetic peptide
RASQSVSSNLA

SEQ ID NO: 320 - protein, 7 aa, Artificial Sequence
  Description of Artificial Sequence Synthetic peptide
GASTRAT

SEQ ID NO: 321 - protein, 9 aa, Artificial Sequence
  Description of Artificial Sequence Synthetic peptide
QQYTVYPPT

SEQ ID NO: 322 - protein, 5 aa, Artificial Sequence
  Description of Artificial Sequence Synthetic peptide
GYYWS

SEQ ID NO: 323 - protein, 16 aa, Artificial Sequence
  Description of Artificial Sequence Synthetic peptide
EIDHSGSTNYNPSLKS

SEQ ID NO: 324 - protein, 11 aa, Artificial Sequence
  Description of Artificial Sequence Synthetic peptide
QGIHGLRYFDL

SEQ ID NO: 325 - protein, 11 aa, Artificial Sequence
  Description of Artificial Sequence Synthetic peptide
RASQSVSSYLA

SEQ ID NO: 326 - protein, 7 aa, Artificial Sequence
  Description of Artificial Sequence Synthetic peptide
DASNRAT

SEQ ID NO: 327 - protein, 9 aa, Artificial Sequence
  Description of Artificial Sequence Synthetic peptide
QQDHNFPYT

SEQ ID NO: 328 - protein, 5 aa, Artificial Sequence
  Description of Artificial Sequence Synthetic peptide
SYWMS

SEQ ID NO: 329 - protein, 17 aa, Artificial Sequence
  Description of Artificial Sequence Synthetic peptide
NINQDGSEKYYVDSVKG

SEQ ID NO: 330 - protein, 12 aa, Artificial Sequence
  Description of Artificial Sequence Synthetic peptide
EANYYGNVGDDY

SEQ ID NO: 331 - protein, 11 aa, Artificial Sequence
  Description of Artificial Sequence Synthetic peptide
RASQSISSYLN

SEQ ID NO: 332 - protein, 7 aa, Artificial Sequence
  Description of Artificial Sequence Synthetic peptide
AASSLQS

SEQ ID NO: 333 - protein, 9 aa, Artificial Sequence
  Description of Artificial Sequence Synthetic peptide
QQQYVTPIT

SEQ ID NO: 334 - protein, 5 aa, Artificial Sequence
  Description of Artificial Sequence Synthetic peptide
SYWMS

SEQ ID NO: 335 - protein, 17 aa, Artificial Sequence
  Description of Artificial Sequence Synthetic peptide
NINQDGSEKYYVDSVKG

SEQ ID NO: 336 - protein, 12 aa, Artificial Sequence
  Description of Artificial Sequence Synthetic peptide
EGGDSWYHAFDI

SEQ ID NO: 337 - protein, 11 aa, Artificial Sequence
  Description of Artificial Sequence Synthetic peptide
RASQGISSWLA

SEQ ID NO: 338 - protein, 7 aa, Artificial Sequence
  Description of Artificial Sequence Synthetic peptide
AASNLQS

SEQ ID NO: 339 - protein, 9 aa, Artificial Sequence
  Description of Artificial Sequence Synthetic peptide
QQKLSLPLT

SEQ ID NO: 340 - protein, 7 aa, Artificial Sequence
  Description of Artificial Sequence Synthetic peptide
SGGYYWS

SEQ ID NO: 341 - protein, 16 aa, Artificial Sequence
  Description of Artificial Sequence Synthetic peptide
SIYYSGSTYYNPSLKS

SEQ ID NO: 342 - protein, 13 aa, Artificial Sequence
  Description of Artificial Sequence Synthetic peptide
DRLDYSYNYGMDV

SEQ ID NO: 343 - protein, 11 aa, Artificial Sequence
  Description of Artificial Sequence Synthetic peptide
RASQSISSYLN

SEQ ID NO: 344 - protein, 7 aa, Artificial Sequence
  Description of Artificial Sequence Synthetic peptide
GASSLQS

SEQ ID NO: 345 - protein, 9 aa, Artificial Sequence
  Description of Artificial Sequence Synthetic peptide
QQVYSAPFT

SEQ ID NO: 346 - protein, 6 aa, Artificial Sequence
  Description of Artificial Sequence Synthetic peptide
SGYYWG

SEQ ID NO: 347 - protein, 16 aa, Artificial Sequence
  Description of Artificial Sequence Synthetic peptide
SIYHSGSTNYNPSLKS

SEQ ID NO: 348 - protein, 12 aa, Artificial Sequence
  Description of Artificial Sequence Synthetic peptide
LPPWFGFSYFDL

SEQ ID NO: 349 - protein, 11 aa, Artificial Sequence
  Description of Artificial Sequence Synthetic peptide
RASQSVSSYLA

SEQ ID NO: 350 - protein, 7 aa, Artificial Sequence
  Description of Artificial Sequence Synthetic peptide
DASNRAT

SEQ ID NO: 351 - protein, 9 aa, Artificial Sequence
  Description of Artificial Sequence Synthetic peptide
QQVDNYPPT

SEQ ID NO: 352 - protein, 5 aa, Artificial Sequence
  Description of Artificial Sequence Synthetic peptide
SYWMS

SEQ ID NO: 353 - protein, 17 aa, Artificial Sequence
  Description of Artificial Sequence Synthetic peptide
NIKQDGSEKYYVDSVKG

SEQ ID NO: 354 - protein, 14 aa, Artificial Sequence
  Description of Artificial Sequence Synthetic peptide
DVGPGIAYQGHFDY

SEQ ID NO: 355 - protein, 11 aa, Artificial Sequence
  Description of Artificial Sequence Synthetic peptide
RASQSISSYLN

SEQ ID NO: 356 - protein, 7 aa, Artificial Sequence
  Description of Artificial Sequence Synthetic peptide
AASSLQS

SEQ ID NO: 357 - protein, 9 aa, Artificial Sequence
  Description of Artificial Sequence Synthetic peptide
QQVYDTPLT

SEQ ID NO: 358 - protein, 7 aa, Artificial Sequence
  Description of Artificial Sequence Synthetic peptide
SSSYYWG

SEQ ID NO: 359 - protein, 16 aa, Artificial Sequence
  Description of Artificial Sequence Synthetic peptide
SIYYSGSTYYNPSLKS

SEQ ID NO: 360 - protein, 11 aa, Artificial Sequence
  Description of Artificial Sequence Synthetic peptide
ETAHDVHGMDV

SEQ ID NO: 361 - protein, 11 aa, Artificial Sequence
  Description of Artificial Sequence Synthetic peptide
RASQSVSSYLA

SEQ ID NO: 362 - protein, 7 aa, Artificial Sequence
  Description of Artificial Sequence Synthetic peptide
DASNRAT

SEQ ID NO: 363 - protein, 8 aa, Artificial Sequence
  Description of Artificial Sequence Synthetic peptide
QQYDNLPT

SEQ ID NO: 364 - protein, 5 aa, Artificial Sequence
  Description of Artificial Sequence Synthetic peptide
SYAIS

SEQ ID NO: 365 - protein, 17 aa, Artificial Sequence
  Description of Artificial Sequence Synthetic peptide
SIIPIFGTANYAQKFQG

SEQ ID NO: 366 - protein, 14 aa, Artificial Sequence
  Description of Artificial Sequence Synthetic peptide
EVGYGWYTKIAFDI

SEQ ID NO: 367 - protein, 11 aa, Artificial Sequence
  Description of Artificial Sequence Synthetic peptide
RASQSVSSYLA

SEQ ID NO: 368 - protein, 7 aa, Artificial Sequence
  Description of Artificial Sequence Synthetic peptide
DASKRAT

SEQ ID NO: 369 - protein, 9 aa, Artificial Sequence
  Description of Artificial Sequence Synthetic peptide
QQSSNHPST

SEQ ID NO: 370 - protein, 5 aa, Artificial Sequence
  Description of Artificial Sequence Synthetic peptide
SYYMH

SEQ ID NO: 371 - protein, 17 aa, Artificial Sequence
  Description of Artificial Sequence Synthetic peptide
IINPSGGSTTYAQKFQG

SEQ ID NO: 372 - protein, 14 aa, Artificial Sequence
  Description of Artificial Sequence Synthetic peptide
EAADGFVGERYFDL

SEQ ID NO: 373 - protein, 16 aa, Artificial Sequence
  Description of Artificial Sequence Synthetic peptide
RSSQSLLHSNGYNYLD

SEQ ID NO: 374 - protein, 7 aa, Artificial Sequence
  Description of Artificial Sequence Synthetic peptide
LGSNRAS

SEQ ID NO: 375 - protein, 9 aa, Artificial Sequence
  Description of Artificial Sequence Synthetic peptide
MQALGVPLT

SEQ ID NO: 376 - protein, 5 aa, Artificial Sequence
  Description of Artificial Sequence Synthetic peptide
GYYMH

SEQ ID NO: 377 - protein, 17 aa, Artificial Sequence
  Description of Artificial Sequence Synthetic peptide
MINPYGGSTRYAQKFQG

SEQ ID NO: 378 - protein, 14 aa, Artificial Sequence
  Description of Artificial Sequence Synthetic peptide
EAADGFVGERYFDL

SEQ ID NO: 379 - protein, 16 aa, Artificial Sequence
  Description of Artificial Sequence Synthetic peptide
RSSQSLLYSNGYNYLD

SEQ ID NO: 380 - protein, 7 aa, Artificial Sequence
  Description of Artificial Sequence Synthetic peptide
LGSNRAS

SEQ ID NO: 381 - protein, 9 aa, Artificial Sequence
  Description of Artificial Sequence Synthetic peptide
MQDVALPIT

SEQ ID NO: 382 - protein, 5 aa, Artificial Sequence
  Description of Artificial Sequence Synthetic peptide
IYYMH

SEQ ID NO: 383 - protein, 17 aa, Artificial Sequence
  Description of Artificial Sequence Synthetic peptide
IINPSSGSTVYAQKFQG

SEQ ID NO: 384 - protein, 11 aa, Artificial Sequence
  Description of Artificial Sequence Synthetic peptide
GAGYDDEDMDV

SEQ ID NO: 385 - protein, 11 aa, Artificial Sequence
  Description of Artificial Sequence Synthetic peptide
RASQGIDSWLA

SEQ ID NO: 386 - protein, 7 aa, Artificial Sequence
  Description of Artificial Sequence Synthetic peptide
AASSLQS

SEQ ID NO: 387 - protein, 9 aa, Artificial Sequence
  Description of Artificial Sequence Synthetic peptide
QQAHSYPLT

SEQ ID NO: 388 - protein, 5 aa, Artificial Sequence
  Description of Artificial Sequence Synthetic peptide
GYWMS

SEQ ID NO: 389 - protein, 17 aa, Artificial Sequence
  Description of Artificial Sequence Synthetic peptide
NINQDGSEEYYVDSVKG

SEQ ID NO: 390 - protein, 12 aa, Artificial Sequence
  Description of Artificial Sequence Synthetic peptide
EANYYGNVGDDY

SEQ ID NO: 391 - protein, 11 aa, Artificial Sequence
  Description of Artificial Sequence Synthetic peptide
RASQSIYNYLN

SEQ ID NO: 392 - protein, 7 aa, Artificial Sequence
  Description of Artificial Sequence Synthetic peptide
AASNLHS

SEQ ID NO: 393 - protein, 9 aa, Artificial Sequence
  Description of Artificial Sequence Synthetic peptide
QQAFHVPIT

SEQ ID NO: 394 - protein, 5 aa, Artificial Sequence
  Description of Artificial Sequence Synthetic peptide
GYWMS

SEQ ID NO: 395 - protein, 17 aa, Artificial Sequence
  Description of Artificial Sequence Synthetic peptide
NINQDGSEVYYVDSVKG

SEQ ID NO: 396 - protein, 12 aa, Artificial Sequence
  Description of Artificial Sequence Synthetic peptide
EANYYGNVGDDY

SEQ ID NO: 397 - protein, 11 aa, Artificial Sequence
  Description of Artificial Sequence Synthetic peptide
RASQSIYNYLN

SEQ ID NO: 398 - protein, 7 aa, Artificial Sequence
  Description of Artificial Sequence Synthetic peptide
AASSTQS

SEQ ID NO: 399 - protein, 9 aa, Artificial Sequence
  Description of Artificial Sequence Synthetic peptide
QQAFHVPIT

SEQ ID NO: 400 - protein, 5 aa, Artificial Sequence
  Description of Artificial Sequence Synthetic peptide
SYWMS

SEQ ID NO: 401 - protein, 17 aa, Artificial Sequence
  Description of Artificial Sequence Synthetic peptide
NINQDGSEVYYVDSVKG

SEQ ID NO: 402 - protein, 14 aa, Artificial Sequence
  Description of Artificial Sequence Synthetic peptide
DVGPGIAYQGHFDY

SEQ ID NO: 403 - protein, 11 aa, Artificial Sequence
  Description of Artificial Sequence Synthetic peptide
RASQSIYYYLN

SEQ ID NO: 404 - protein, 7 aa, Artificial Sequence
  Description of Artificial Sequence Synthetic peptide
AASSRQS

SEQ ID NO: 405 - protein, 9 aa, Artificial Sequence
  Description of Artificial Sequence Synthetic peptide
QQVYDTPLT

SEQ ID NO: 406 - protein, 5 aa, Artificial Sequence
  Description of Artificial Sequence Synthetic peptide
NYYMH

SEQ ID NO: 407 - protein, 17 aa, Artificial Sequence
  Description of Artificial Sequence Synthetic peptide
WINPFSGGTRYAQKFQG

SEQ ID NO: 408 - protein, 12 aa, Artificial Sequence
  Description of Artificial Sequence Synthetic peptide
DVGSSAYYYMDV

SEQ ID NO: 409 - protein, 11 aa, Artificial Sequence
  Description of Artificial Sequence Synthetic peptide
EASKGISSWLA

SEQ ID NO: 410 - protein, 7 aa, Artificial Sequence
  Description of Artificial Sequence Synthetic peptide
AASDLQS

SEQ ID NO: 411 - protein, 9 aa, Artificial Sequence
  Description of Artificial Sequence Synthetic peptide
QQAFLFPPT

SEQ ID NO: 412 - protein, 5 aa, Artificial Sequence
  Description of Artificial Sequence Synthetic peptide
SYWIG

SEQ ID NO: 413 - protein, 17 aa, Artificial Sequence
  Description of Artificial Sequence Synthetic peptide
SIYPGDSDTRYSPSFQG

SEQ ID NO: 414 - protein, 13 aa, Artificial Sequence
  Description of Artificial Sequence Synthetic peptide
ELAYGDYKGGVDY

SEQ ID NO: 415 - protein, 12 aa, Artificial Sequence
  Description of Artificial Sequence Synthetic peptide
RASQSVSSSFLA

SEQ ID NO: 416 - protein, 7 aa, Artificial Sequence
  Description of Artificial Sequence Synthetic peptide
GASSRAT

SEQ ID NO: 417 - protein, 9 aa, Artificial Sequence
  Description of Artificial Sequence Synthetic peptide
QQLDSPPPT

SEQ ID NO: 418 - protein, 117 aa, Artificial Sequence
  Description of Artificial Sequence Synthetic polypeptide
QVQLVQSGAEVKKPGSSVKVSCKASGGTFSDYAISWVRQAPGQGLEWMGRIIPILGVADYAQKFQGRVTITADKSTRTAYMELSSLRSEDTAVYYCARNWADAFDIWGQGTMVTVSS

SEQ ID NO: 419 - protein, 106 aa, Artificial Sequence
  Description of Artificial Sequence Synthetic polypeptide
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGQGTKLEIK

SEQ ID NO: 420 - protein, 116 aa, Artificial Sequence
  Description of Artificial Sequence Synthetic polypeptide
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 421 - protein, 111 aa, Artificial Sequence
  Description of Artificial Sequence Synthetic polypeptide
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 422 - protein, 5 aa, Artificial Sequence
  Description of Artificial Sequence Synthetic peptide
DYAIS

SEQ ID NO: 423 - protein, 17 aa, Artificial Sequence
  Description of Artificial Sequence Synthetic peptide
RIIPILGVADYAQKFQG

SEQ ID NO: 424 - protein, 8 aa, Artificial Sequence
  Description of Artificial Sequence Synthetic peptide
NWADAFDI

SEQ ID NO: 425 - protein, 11 aa, Artificial Sequence
  Description of Artificial Sequence Synthetic peptide
RASQGISSVLA

SEQ ID NO: 426 - protein, 7 aa, Artificial Sequence
  Description of Artificial Sequence Synthetic peptide
DASSLES

SEQ ID NO: 427 - protein, 8 aa, Artificial Sequence
  Description of Artificial Sequence Synthetic peptide
QQFDSSIT

SEQ ID NO: 428 - protein, 5 aa, Artificial Sequence
  Description of Artificial Sequence Synthetic peptide
DYNMH

SEQ ID NO: 429 - protein, 17 aa, Artificial Sequence
  Description of Artificial Sequence Synthetic peptide
YIYPYNGGTGYNQKFKS

SEQ ID NO: 430 - protein, 7 aa, Artificial Sequence
  Description of Artificial Sequence Synthetic peptide
GRPAMDY

SEQ ID NO: 431 - protein, 15 aa, Artificial Sequence
  Description of Artificial Sequence Synthetic peptide
RASESVDNYGISFMN

SEQ ID NO: 432 - protein, 7 aa, Artificial Sequence
  Description of Artificial Sequence Synthetic peptide
AASNQGS

SEQ ID NO: 433 - protein, 9 aa, Artificial Sequence
  Description of Artificial Sequence Synthetic peptide
QQSKEVPWT

SEQ ID NO: 434 - protein, 5 aa, Artificial Sequence
  Description of Artificial Sequence Synthetic peptide
SYGMS

SEQ ID NO: 435 - protein, 19 aa, Artificial Sequence
  Description of Artificial Sequence Synthetic peptide
EGGPYYDSSGYFVYYGMDV

SEQ ID NO: 436 - protein, 9 aa, Artificial Sequence
  Description of Artificial Sequence Synthetic peptide
PLNAGELDV

SEQ ID NO: 437 - protein, 5 aa, Artificial Sequence
  Description of Artificial Sequence Synthetic peptide
DYYMH

SEQ ID NO: 438 - protein, 14 aa, Artificial Sequence
  Description of Artificial Sequence Synthetic peptide
EAADGFVGERYFDL

SEQ ID NO: 439 - protein, 9 aa, Artificial Sequence
  Description of Artificial Sequence Synthetic peptide
PLNAGELDV

SEQ ID NO: 440 - protein, 5 aa, Artificial Sequence
  Description of Artificial Sequence Synthetic peptide
TYYMH

SEQ ID NO: 441 - protein, 11 aa, Artificial Sequence
  Description of Artificial Sequence Synthetic peptide
GAGYDDEDMDV

SEQ ID NO: 442 - protein, 5 aa, Artificial Sequence
  Description of Artificial Sequence Synthetic peptide
SYAMS

SEQ ID NO: 443 - protein, 19 aa, Artificial Sequence
  Description of Artificial Sequence Synthetic peptide
EGGPYYDSSGYFVYYGMDV

SEQ ID NO: 444 - protein, 14 aa, Artificial Sequence
  Description of Artificial Sequence Synthetic peptide
DVGPGIAYQGHFDY

SEQ ID NO: 445 - protein, 7 aa, Artificial Sequence
  Description of Artificial Sequence Synthetic peptide
STDYYWG

SEQ ID NO: 446 - protein, 11 aa, Artificial Sequence
  Description of Artificial Sequence Synthetic peptide
ETAHDVHGMDV

SEQ ID NO: 447 - protein, 255 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQASSSPPTFGCGTKVEIKGGGGSGGGGSGGGGSGGGGSEVQLLESGGGLVQPGGSLRLSCAASGFTFSSYAMSWVRQAPGKCLEWVSAISASGGSTYYADSVKGRFTISRDNSKNTLYLQMNSLRAEDTAVYYCARPRAYYDSSGFKVNYGMDVWGQGTTVTVSS

SEQ ID NO: 448 - protein, 255 aa, Artificial Sequence
  Description of Artificial Sequence Synthetic polypeptide
EVQLLESGGGLVQPGGSLRLSCAASGFTFSSYAMSWVRQAPGKCLEWVSAISASGGSTYYADSVKGRFTISRDNSKNTLYLQMNSLRAEDTAVYYCARPRAYYDSSGFKVNYGMDVWGQGTTVTVSSGGGGSGGGGSGGGGSGGGGSEIVLTQSPGTLSLSPGERATLSCRASQSVSSSFLAWYQQKPGQAPRLLIYGASSRATGIPDRFSGSGSGTDFTLTISRLEPEDFAVYYCQQASSSPPTFGCGTKVEIK

SEQ ID NO: 449 - protein, 251 aa, Artificial Sequence
  Description of Artificial Sequence Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQQHSSAPPTFGCGTKVEIKGGGGSGGGGSGGGGSGGGGSEVQLLESGGGLVQPGGSLRLSCAASGFTFSSYAMSWVRQAPGKCLEWVSGISGSGGSTYYADSVKGRFTISRDNSKNTLYLQMNSLRAEDTAVYYCAREGHSSSYYDHAFDIWGQGTMVTVSS

SEQ ID NO: 450 - protein, 251 aa, Artificial Sequence
  Description of Artificial Sequence Synthetic polypeptide
EVQLLESGGGLVQPGGSLRLSCAASGFTFSSYAMSWVRQAPGKCLEWVSGISGSGGSTYYADSVKGRFTISRDNSKNTLYLQMNSLRAEDTAVYYCAREGHSSSYYDHAFDIWGQGTMVTVSSGGGGSGGGGSGGGGSGGGGSEIVLTQSPGTLSLSPGERATLSCRASQSVSSDYLAWYQQKPGQAPRLLIYGASSRATGIPDRFSGSGSGTDFTLTISRLEPEDFAVYYCQQHSSAPPTFGCGTKVEIK

SEQ ID NO: 451 - protein, 250 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYTVYPPTFGCGTKVEIKGGGGSGGGGSGGGGSGGGGSQVQLQESGPGLVKPSETLSLTCTVSGGSISSYYWSWIRQPPGKCLEWIGSIYYSGSTNYNPSLKSRVTISVDTSKNQFSLKLSSVTAADTAVYYCARVGGVYSTIETYGMDVWGQGTTVTVSS

SEQ ID NO: 452 - protein, 250 aa, Artificial Sequence
  Description of Artificial Sequence Synthetic polypeptide
QVQLQESGPGLVKPSETLSLTCTVSGGSISSYYWSWIRQPPGKCLEWIGSIYYSGSTNYNPSLKSRVTISVDTSKNQFSLKLSSVTAADTAVYYCARVGGVYSTIETYGMDVWGQGTTVTVSSGGGGSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYTVYPPTFGCGTKVEIK

SEQ ID NO: 453 - protein, 246 aa, Artificial Sequence
  Description of Artificial Sequence Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQDHNFPYTFGCGTKVEIKGGGGSGGGGSGGGGSGGGGSQVQLQQWGAGLLKPSETLSLTCAVYGGSFSGYYWSWIRQPPGKCLEWIGEIDHSGSTNYNPSLKSRVTISVDTSKNQFSLKLSSVTAADTAVYYCARQGIHGLRYFDLWGRGTLVTVSS

SEQ ID NO: 454 - protein, 246 aa, Artificial Sequence
  Description of Artificial Sequence Synthetic polypeptide
QVQLQQWGAGLLKPSETLSLTCAVYGGSFSGYYWSWIRQPPGKCLEWIGEIDHSGSTNYNPSLKSRVTISVDTSKNQFSLKLSSVTAADTAVYYCARQGIHGLRYFDLWGRGTLVTVSSGGGGSGGGGSGGGGSGGGGSEIVMTQSPATLSLSPGERATLSCRASQSVSSYLAWYQQKPGQAPRLLIYDASNRATGIPARFSGSGSGTDFTLTISSLEPEDFAVYYCQQDHNFPYTFGCGTKVEIK

SEQ ID NO: 455 - protein, 248 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQQYVTPITFGCGTKVEIKGGGGSGGGGSGGGGSGGGGSEVQLVESGGGLVQPGGSLRLSCAASGFTFSSYWMSWVRQAPGKCLEWVANINQDGSEKYYVDSVKGRFTISRDNAKNSLYLQMNSLRAEDTAVYYCAREANYYGNVGDDYWGQGTLVTVSS

SEQ ID NO: 456 - protein, 248 aa, Artificial Sequence
  Description of Artificial Sequence Synthetic polypeptide
EVQLVESGGGLVQPGGSLRLSCAASGFTFSSYWMSWVRQAPGKCLEWVANINQDGSEKYYVDSVKGRFTISRDNAKNSLYLQMNSLRAEDTAVYYCAREANYYGNVGDDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQQYVTPITFGCGTKVEIK

SEQ ID NO: 457 - protein, 248 aa, Artificial Sequence
  Description of Artificial Sequence Synthetic polypeptide
DIQMTQSPSSVSASVGDRVTITCRASQGISSWLAWYQQKPGKAPKLLIYAASNLQSGVPSRFSGSGSGTDFTLTISSLQPEDFATYYCQQKLSLPLTFGCGTKVEIKGGGGSGGGGSGGGGSGGGGSEVQLVESGGGLVQPGGSLRLSCAASGFTFSSYWMSWVRQAPGKCLEWVANINQDGSEKYYVDSVKGRFTISRDNAKNSLYLQMNSLRAEDTAVYYCAREGGDSWYHAFDIWGQGTMVTVSS

SEQ ID NO: 458 - protein, 248 aa, Artificial Sequence
  Description of Artificial Sequence Synthetic polypeptide
EVQLVESGGGLVQPGGSLRLSCAASGFTFSSYWMSWVRQAPGKCLEWVANINQDGSEKYYVDSVKGRFTISRDNAKNSLYLQMNSLRAEDTAVYYCAREGGDSWYHAFDIWGQGTMVTVSSGGGGSGGGGSGGGGSGGGGSDIQMTQSPSSVSASVGDRVTITCRASQGISSWLAWYQQKPGKAPKLLIYAASNLQSGVPSRFSGSGSGTDFTLTISSLQPEDFATYYCQQKLSLPLTFGCGTKVEIK

SEQ ID NO: 459 - protein, 250 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVYSAPFTFGCGTKVEIKGGGGSGGGGSGGGGSGGGGSQVQLQESGPGLVKPSQTLSLTCTVSGGSISSGGYYWSWIRQHPGKCLEWIGSIYYSGSTYYNPSLKSRVTISVDTSKNQFSLKLSSVTAADTAVYYCARDRLDYSYNYGMDVWGQGTTVTVSS

SEQ ID NO: 460 - protein, 250 aa, Artificial Sequence
  Description of Artificial Sequence Synthetic polypeptide
QVQLQESGPGLVKPSQTLSLTCTVSGGSISSGGYYWSWIRQHPGKCLEWIGSIYYSGSTYYNPSLKSRVTISVDTSKNQFSLKLSSVTAADTAVYYCARDRLDYSYNYGMDVWGQGTTVTVSSGGGGSGGGGSGGGGSGGGGSDIQMTQSPSSLSASVGDRVTITCRASQSISSYLNWYQQKPGKAPKLLIYGASSLQSGVPSRFSGSGSGTDFTLTISSLQPEDFATYYCQQVYSAPFTFGCGTKVEIK

SEQ ID NO: 461 - protein, 248 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQVDNYPPTFGCGTKVEIKGGGGSGGGGSGGGGSGGGGSQVQLQESGPGLVKPSETLSLTCAVSGYSISSGYYWGWIRQPPGKCLEWIGSIYHSGSTNYNPSLKSRVTISVDTSKNQFSLKLSSVTAADTAVYYCARLPPWFGFSYFDLWGRGTLVTVSS

SEQ ID NO: 462 - protein, 248 aa, Artificial Sequence
  Description of Artificial Sequence Synthetic polypeptide
QVQLQESGPGLVKPSETLSLTCAVSGYSISSGYYWGWIRQPPGKCLEWIGSIYHSGSTNYNPSLKSRVTISVDTSKNQFSLKLSSVTAADTAVYYCARLPPWFGFSYFDLWGRGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQVDNYPPTFGCGTKVEIK

SEQ ID NO: 463 - protein, 250 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQVYDTPLTFGCGTKVEIKGGGGSGGGGSGGGGSGGGGSEVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARDVGPGIAYQGHFDYWGQGTLVTVSS

SEQ ID NO: 464 - protein, 250 aa, Artificial Sequence
  Description of Artificial Sequence Synthetic polypeptide
EVQLVESGGGLVQPGGSLRLSCAASGFTFSSYWMSWVRQAPGKCLEWVANIKQDGSEKYYVDSVKGRFTISRDNAKNSLYLQMNSLRAEDTAVYYCARDVGPGIAYQGHFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQVYDTPLTFGCGTKVEIK

SEQ ID NO: 465 - protein, 247 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQYDNLPTFGCGTKVEIKGGGGSGGGGSGGGGSGGGGSQLQLQESGPGLVKPSETLSLTCTVSGGSISSSSYYWGWIRQPPGKCLEWIGSIYYSGSTYYNPSLKSRVTISVDTSKNQFSLKLSSVTAADTAVYYCARETAHDVHGMDVWGQGTTVTVSS

SEQ ID NO: 466 - protein, 247 aa, Artificial Sequence
  Description of Artificial Sequence Synthetic polypeptide
QLQLQESGPGLVKPSETLSLTCTVSGGSISSSSYYWGWIRQPPGKCLEWIGSIYYSGSTYYNPSLKSRVTISVDTSKNQFSLKLSSVTAADTAVYYCARETAHDVHGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQYDNLPTFGCGTKVEIK

SEQ ID NO: 467 - protein, 250 aa, Artificial Sequence
  Description of Artificial Sequence Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQSSNHPSTFGCGTKVEIKGGGGSGGGGSGGGGSGGGGSQVQLVQSGAEVKKPGSSVKVSCKASGGTFSSYAISWVRQAPGQCLEWMGSIIPIFGTANYAQKFQGRVTITADESTSTAYMELSSLRSEDTAVYYCAREVGYGWYTKIAFDIWGQGTMVTVSS

SEQ ID NO: 468 - protein, 250 aa, Artificial Sequence
  Description of Artificial Sequence Synthetic polypeptide
QVQLVQSGAEVKKPGSSVKVSCKASGGTFSSYAISWVRQAPGQCLEWMGSIIPIFGTANYAQKFQGRVTITADESTSTAYMELSSLRSEDTAVYYCAREVGYGWYTKIAFDIWGQGTMVTVSSGGGGSGGGGSGGGGSGGGGSEIVLTQSPATLSLSPGERATLSCRASQSVSSYLAWYQQKPGQAPRLLIYDASKRATGIPARFSGSGSGTDFTLTISSLEPEDFAVYYCQQSSNHPSTFGCGTKVEIK

SEQ ID NO: 469 - protein, 255 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGVPLTFGCGTKVEIKGGGGSGGGGSGGGGSGGGGSQVQLVQSGAEVKKPGASVKVSCKASGYTFTSYYMHWVRQAPGQCLEWMGIINPSGGSTTYAQKFQGRVTMTRDTSTSTVYMELSSLRSEDTAVYYCAREAADGFVGERYFDLWGRGTLVTVSS

SEQ ID NO: 470 - protein, 255 aa, Artificial Sequence
  Description of Artificial Sequence Synthetic polypeptide
QVQLVQSGAEVKKPGASVKVSCKASGYTFTSYYMHWVRQAPGQCLEWMGIINPSGGSTTYAQKFQGRVTMTRDTSTSTVYMELSSLRSEDTAVYYCAREAADGFVGERYFDLWGRGTLVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGVPLTFGCGTKVEIK

SEQ ID NO: 471 - protein, 255 aa, Artificial Sequence
  Description of Artificial Sequence Synthetic polypeptide
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIKGGGGSGGGGSGGGGSGGGGSQVQLVQSGAEVKKPGASVKVSCKASGYTFSGYYMHWVRQAPGQCLEWMGMINPYGGSTRYAQKFQGRVTMTRDTSTSTVYMELSSLRSEDTAVYYCAREAADGFVGERYFDLWGRGTLVTVSS

SEQ ID NO: 472 - protein, 255 aa, Artificial Sequence
  Description of Artificial Sequence Synthetic polypeptide
QVQLVQSGAEVKKPGASVKVSCKASGYTFSGYYMHWVRQAPGQCLEWMGMINPYGGSTRYAQKFQGRVTMTRDTSTSTVYMELSSLRSEDTAVYYCAREAADGFVGERYFDLWGRGTLVTVSSGGGGSGGGGSGGGGSGGGGSDIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQDVALPITFGCGTKVEIK

SEQ ID NO: 473 - protein, 247 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIKGGGGSGGGGSGGGGSGGGGSQVQLVQSGAEVKKPGASVKVSCKASGYTFEIYYMHWVRQAPGQCLEWMGIINPSSGSTVYAQKFQGRVTMTRDTSTSTVYMELSSLRSEDTAVYYCARGAGYDDEDMDVWGKGTTVTVSS

SEQ ID NO: 474 - protein, 247 aa, Artificial Sequence
  Description of Artificial Sequence Synthetic polypeptide
QVQLVQSGAEVKKPGASVKVSCKASGYTFEIYYMHWVRQAPGQCLEWMGIINPSSGSTVYAQKFQGRVTMTRDTSTSTVYMELSSLRSEDTAVYYCARGAGYDDEDMDVWGKGTTVTVSSGGGGSGGGGSGGGGSGGGGSDIQMTQSPSSVSASVGDRVTITCRASQGIDSWLAWYQQKPGKAPKLLIYAASSLQSGVPSRFSGSGSGTDFTLTISSLQPEDFATYYCQQAHSYPLTFGCGTKVEIK

SEQ ID NO: 475 - protein, 248 aa, Artificial Sequence
  Description of Artificial Sequence Synthetic polypeptide
DIQMTQSPSSLSASVGDRVTITCRASQSIYNYLNWYQQKPGKAPKLLIYAASNLHSGVPSRFSGSGSGTDFTLTISSLQPEDFATYYCQQAFHVPITFGCGTKVEIKGGGGSGGGGSGGGGSGGGGSEVQLVESGGGLVQPGGSLRLSCAASGFTFGGYWMSWVRQAPGKCLEWVANINQDGSEEYYVDSVKGRFTISRDNAKNSLYLQMNSLRAEDTAVYYCAREANYYGNVGDDYWGQGTLVTVSS

SEQ ID NO: 476 - protein, 248 aa, Artificial Sequence
  Description of Artificial Sequence Synthetic polypeptide
EVQLVESGGGLVQPGGSLRLSCAASGFTFGGYWMSWVRQAPGKCLEWVANINQDGSEEYYVDSVKGRFTISRDNAKNSLYLQMNSLRAEDTAVYYCAREANYYGNVGDDYWGQGTLVTVSSGGGGSGGGGSGGGGSGGGGSDIQMTQSPSSLSASVGDRVTITCRASQSIYNYLNWYQQKPGKAPKLLIYAASNLHSGVPSRFSGSGSGTDFTLTISSLQPEDFATYYCQQAFHVPITFGCGTKVEIK

SEQ ID NO: 477 - protein, 248 aa, Artificial Sequence
  Description of Artificial Sequence Synthetic polypeptide
DIQMTQSPSSLSASVGDRVTITCRASQSIYNYLNWYQQKPGKAPKLLIYAASSTQSGVPSRFSGSGSGTDFTLTISSLQPEDFATYYCQQAFHVPITFGCGTKVEIKGGGGSGGGGSGGGGSGGGGSEVQLVESGGGLVQPGGSLRLSCAASGFTFPGYWMSWVRQAPGKCLEWVANINQDGSEVYYVDSVKGRFTISRDNAKNSLYLQMNSLRAEDTAVYYCAREANYYGNVGDDYWGQGTLVTVSS

SEQ ID NO: 478 - protein, 248 aa, Artificial Sequence
  Description of Artificial Sequence Synthetic polypeptide
EVQLVESGGGLVQPGGSLRLSCAASGFTFPGYWMSWVRQAPGKCLEWVANINQDGSEVYYVDSVKGRFTISRDNAKNSLYLQMNSLRAEDTAVYYCAREANYYGNVGDDYWGQGTLVTVSSGGGGSGGGGSGGGGSGGGGSDIQMTQSPSSLSASVGDRVTITCRASQSIYNYLNWYQQKPGKAPKLLIYAASSTQSGVPSRFSGSGSGTDFTLTISSLQPEDFATYYCQQAFHVPITFGCGTKVEIK

SEQ ID NO: 479 - protein, 250 aa, Artificial Sequence
  Description of Artificial Sequence Synthetic polypeptide
DIQMTQSPSSLSASVGDRVTITCRASQSIYYYLNWYQQKPGKAPKLLIYAASSRQSGVPSRFSGSGSGTDFTLTISSLQPEDFATYYCQQVYDTPLTFGCGTKVEIKGGGGSGGGGSGGGGSGGGGSEVQLVESGGGLVQPGGSLRLSCAASGFTFSSYWMSWVRQAPGKCLEWVANINQDGSEVYYVDSVKGRFTISRDNAKNSLYLQMNSLRAEDTAVYYCARDVGPGIAYQGHFDYWGQGTLVTVSS

SEQ ID NO: 480 - protein, 250 aa, Artificial Sequence
  Description of Artificial Sequence Synthetic polypeptide
EVQLVESGGGLVQPGGSLRLSCAASGFTFSSYWMSWVRQAPGKCLEWVANINQDGSEVYYVDSVKGRFTISRDNAKNSLYLQMNSLRAEDTAVYYCARDVGPGIAYQGHFDYWGQGTLVTVSSGGGGSGGGGSGGGGSGGGGSDIQMTQSPSSLSASVGDRVTITCRASQSIYYYLNWYQQKPGKAPKLLIYAASSRQSGVPSRFSGSGSGTDFTLTISSLQPEDFATYYCQQVYDTPLTFGCGTKVEIK

SEQ ID NO: 481 - protein, 248 aa, Artificial Sequence
  Description of Artificial Sequence Synthetic polypeptide
DIQMTQSPSSVSASVGDRVTITCEASKGISSWLAWYQQKPGKAPKLLIYAASDLQSGVPSRFSGSGSGTDFTLTISSLQPEDFATYYCQQAFLFPPTFGCGTKVEIKGGGGSGGGGSGGGGSGGGGSQVQLVQSGAEVKKPGASVKVSCKASGYTFSNYYMHWVRQAPGQCLEWMGWINPFSGGTRYAQKFQGRVTMTRDTSTSTVYMELSSLRSEDTAVYYCARDVGSSAYYYMDVWGKGTTVTVSS

SEQ ID NO: 482 - protein, 248 aa, Artificial Sequence
  Description of Artificial Sequence Synthetic polypeptide
QVQLVQSGAEVKKPGASVKVSCKASGYTFSNYYMHWVRQAPGQCLEWMGWINPFSGGTRYAQKFQGRVTMTRDTSTSTVYMELSSLRSEDTAVYYCARDVGSSAYYYMDVWGKGTTVTVSSGGGGSGGGGSGGGGSGGGGSDIQMTQSPSSVSASVGDRVTITCEASKGISSWLAWYQQKPGKAPKLLIYAASDLQSGVPSRFSGSGSGTDFTLTISSLQPEDFATYYCQQAFLFPPTFGCGTKVEIK

SEQ ID NO: 483 - protein, 250 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQLDSPPPTFGCGTKVEIKGGGGSGGGGSGGGGSGGGGSEVQLVQSGAEVKKPGESLKISCKGSGYSFTSYWIGWVRQMPGKCLEWMGSIYPGDSDTRYSPSFQGQVTISADKSISTAYLQWSSLKASDTAMYYCARELAYGDYKGGVDYWGQGTLVTVSS

SEQ ID NO: 484 - protein, 250 aa, Artificial Sequence
  Description of Artificial Sequence Synthetic polypeptide
EVQLVQSGAEVKKPGESLKISCKGSGYSFTSYWIGWVRQMPGKCLEWMGSIYPGDSDTRYSPSFQGQVTISADKSISTAYLQWSSLKASDTAMYYCARELAYGDYKGGVDYWGQGTLVTVSSGGGGSGGGGSGGGGSGGGGSEIVLTQSPGTLSLSPGERATLSCRASQSVSSSFLAWYQQKPGQAPRLLIYGASSRATGIPDRFSGSGSGTDFTLTISRLEPEDFAVYYCQQLDSPPPTFGCGTKVEIK

SEQ ID NO: 485 - protein, 243 aa, Artificial Sequence
  Description of Artificial Sequence Synthetic polypeptide
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGCGTKLEIKGGGGSGGGGSGGGGSGGGGSQVQLVQSGAEVKKPGSSVKVSCKASGGTFSDYAISWVRQAPGQCLEWMGRIIPILGVADYAQKFQGRVTITADKSTRTAYMELSSLRSEDTAVYYCARNWADAFDIWGQGTMVTVSS

SEQ ID NO: 486 - protein, 243 aa, Artificial Sequence
  Description of Artificial Sequence Synthetic polypeptide
QVQLVQSGAEVKKPGSSVKVSCKASGGTFSDYAISWVRQAPGQCLEWMGRIIPILGVADYAQKFQGRVTITADKSTRTAYMELSSLRSEDTAVYYCARNWADAFDIWGQGTMVTVSSGGGGSGGGGSGGGGSGGGGSDIQLTQSPSSLSASVGDRVTITCRASQGISSVLAWYQQKPGKAPKLLIYDASSLESGVPSRFSGSGSGTDFTLTISSLQPEDFATYYCQQFDSSITFGCGTKLEIK

SEQ ID NO: 487 - protein, 247 aa, Artificial Sequence
  Description of Artificial Sequence Synthetic polypeptide
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGCGTKVEIKGGGGSGGGGSGGGGSGGGGSQVQLVQSGAEVKKPGSSVKVSCKASGYTFTDYNMHWVRQAPGQCLEWIGYIYPYNGGTGYNQKFKSKATITADESTNTAYMELSSLRSEDTAVYYCARGRPAMDYWGQGTLVTVSS

SEQ ID NO: 488 - protein, 247 aa, Artificial Sequence
  Description of Artificial Sequence Synthetic polypeptide
QVQLVQSGAEVKKPGSSVKVSCKASGYTFTDYNMHWVRQAPGQCLEWIGYIYPYNGGTGYNQKFKSKATITADESTNTAYMELSSLRSEDTAVYYCARGRPAMDYWGQGTLVTVSSGGGGSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGCGTKVEIK

SEQ ID NO: 489 - DNA, 765 bp, Artificial Sequence
  Description of Artificial Sequence Synthetic polynucleotide
gagatcgtgctgacccagagccccggcaccctgagcctgagccccggcgagagggccaccctgagctgcagggccagccagagcgtgagcagcagcttcctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcaggccagcagcagcccccccaccttcggctgcggcaccaaggtggagatcaagggcggcggcggcagcggcggcggcggcagcggcggcggcggcagcggcggcggcggcagcgaggtgcagctgctggagagcggcggcggcctggtgcagcccggcggcagcctgaggctgagctgcgccgccagcggcttcaccttcagcagctacgccatgagctgggtgaggcaggcccccggcaagtgcctggagtgggtgagcgccatcagcgccagcggcggcagcacctactacgccgacagcgtgaagggcaggttcaccatcagcagggacaacagcaagaacaccctgtacctgcagatgaacagcctgagggccgaggacaccgccgtgtactactgcgccaggcccagggcctactacgacagcagcggcttcaaggtgaactacggcatggacgtgtggggccagggcaccaccgtgaccgtgagcagc

SEQ ID NO: 490 - DNA, 765 bp, Artificial Sequence
  Description of Artificial Sequence Synthetic polynucleotide
gaggtgcagctgctggagagcggcggcggcctggtgcagcccggcggcagcctgaggctgagctgcgccgccagcggcttcaccttcagcagctacgccatgagctgggtgaggcaggcccccggcaagtgcctggagtgggtgagcgccatcagcgccagcggcggcagcacctactacgccgacagcgtgaagggcaggttcaccatcagcagggacaacagcaagaacaccctgtacctgcagatgaacagcctgagggccgaggacaccgccgtgtactactgcgccaggcccagggcctactacgacagcagcggcttcaaggtgaactacggcatggacgtgtggggccagggcaccaccgtgaccgtgagcagcggcggcggcggcagcggcggcggcggcagcggcggcggcggcagcggcggcggcggcagcgagatcgtgctgacccagagccccggcaccctgagcctgagccccggcgagagggccaccctgagctgcagggccagccagagcgtgagcagcagcttcctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcaggccagcagcagcccccccaccttcggctgcggcaccaaggtggagatcaag

SEQ ID NO: 491 - DNA, 753 bp, Artificial Sequence
  Description of Artificial Sequence Synthetic polynucleotide
gagatcgtgctgacccagagccccggcaccctgagcctgagccccggcgagagggccaccctgagctgcagggccagccagagcgtgagcagcgactacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagcacagcagcgccccccccaccttcggctgcggcaccaaggtggagatcaagggcggcggcggcagcggcggcggcggcagcggcggcggcggcagcggcggcggcggcagcgaggtgcagctgctggagagcggcggcggcctggtgcagcccggcggcagcctgaggctgagctgcgccgccagcggcttcaccttcagcagctacgccatgagctgggtgaggcaggcccccggcaagtgcctggagtgggtgagcggcatcagcggcagcggcggcagcacctactacgccgacagcgtgaagggcaggttcaccatcagcagggacaacagcaagaacaccctgtacctgcagatgaacagcctgagggccgaggacaccgccgtgtactactgcgccagggagggccacagcagcagctactacgaccacgccttcgacatctggggccagggcaccatggtgaccgtgagcagc

SEQ ID NO: 492 - DNA, 753 bp, Artificial Sequence
  Description of Artificial Sequence Synthetic polynucleotide
gaggtgcagctgctggagagcggcggcggcctggtgcagcccggcggcagcctgaggctgagctgcgccgccagcggcttcaccttcagcagctacgccatgagctgggtgaggcaggcccccggcaagtgcctggagtgggtgagcggcatcagcggcagcggcggcagcacctactacgccgacagcgtgaagggcaggttcaccatcagcagggacaacagcaagaacaccctgtacctgcagatgaacagcctgagggccgaggacaccgccgtgtactactgcgccagggagggccacagcagcagctactacgaccacgccttcgacatctggggccagggcaccatggtgaccgtgagcagcggcggcggcggcagcggcggcggcggcagcggcggcggcggcagcggcggcggcggcagcgagatcgtgctgacccagagccccggcaccctgagcctgagccccggcgagagggccaccctgagctgcagggccagccagagcgtgagcagcgactacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagcacagcagcgccccccccaccttcggctgcggcaccaaggtggagatcaag

SEQ ID NO: 493 - DNA, 750 bp, Artificial Sequence
  Description of Artificial Sequence Synthetic polynucleotide
gagatcgtgatgacccagagccccgccaccctgagcgtgagccccggcgagagggccaccctgagctgcagggccagccagagcgtgagcagcaacctggcctggtaccagcagaagcccggccaggcccccaggctgctgatctacggcgccagcaccagggccaccggcatccccgccaggttcagcggcagcggcagcggcaccgagttcaccctgaccatcagcagcctgcagagcgaggacttcgccgtgtactactgccagcagtacaccgtgtacccccccaccttcggctgcggcaccaaggtggagatcaagggcggcggcggcagcggcggcggcggcagcggcggcggcggcagcggcggcggcggcagccaggtgcagctgcaggagagcggccccggcctggtgaagcccagcgagaccctgagcctgacctgcaccgtgagcggcggcagcatcagcagctactactggagctggatcaggcagccccccggcaagtgcctggagtggatcggcagcatctactacagcggcagcaccaactacaaccccagcctgaagagcagggtgaccatcagcgtggacaccagcaagaaccagttcagcctgaagctgagcagcgtgaccgccgccgacaccgccgtgtactactgcgccagggtgggcggcgtgtacagcaccatcgagacctacggcatggacgtgtggggccagggcaccaccgtgaccgtgagcagc

SEQ ID NO: 494 - DNA, 750 bp, Artificial Sequence
  Description of Artificial Sequence Synthetic polynucleotide
caggtgcagctgcaggagagcggccccggcctggtgaagcccagcgagaccctgagcctgacctgcaccgtgagcggcggcagcatcagcagctactactggagctggatcaggcagccccccggcaagtgcctggagtggatcggcagcatctactacagcggcagcaccaactacaaccccagcctgaagagcagggtgaccatcagcgtggacaccagcaagaaccagttcagcctgaagctgagcagcgtgaccgccgccgacaccgccgtgtactactgcgccagggtgggcggcgtgtacagcaccatcgagacctacggcatggacgtgtggggccagggcaccaccgtgaccgtgagcagcggcggcggcggcagcggcggcggcggcagcggcggcggcggcagcggcggcggcggcagcgagatcgtgatgacccagagccccgccaccctgagcgtgagccccggcgagagggccaccctgagctgcagggccagccagagcgtgagcagcaacctggcctggtaccagcagaagcccggccaggcccccaggctgctgatctacggcgccagcaccagggccaccggcatccccgccaggttcagcggcagcggcagcggcaccgagttcaccctgaccatcagcagcctgcagagcgaggacttcgccgtgtactactgccagcagtacaccgtgtacccccccaccttcggctgcggcaccaaggtggagatcaag

SEQ ID NO: 495 - DNA, 738 bp, Artificial Sequence
  Description of Artificial Sequence Synthetic polynucleotide
gagatcgtgatgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcaggaccacaacttcccctacaccttcggctgcggcaccaaggtggagatcaagggcggcggcggcagcggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcggctactactggagctggatcaggcagccccccggcaagtgcctggagtggatcggcgagatcgaccacagcggcagcaccaactacaaccccagcctgaagagcagggtgaccatcagcgtggacaccagcaagaaccagttcagcctgaagctgagcagcgtgaccgccgccgacaccgccgtgtactactgcgccaggcagggcatccacggcctgaggtacttcgacctgtggggcaggggcaccctggtgaccgtgagcagc

SEQ ID NO: 496 - DNA, 738 bp, Artificial Sequence
  Description of Artificial Sequence Synthetic polynucleotide
caggtgcagctgcagcagtggggcgccggcctgctgaagcccagcgagaccctgagcctgacctgcgccgtgtacggcggcagcttcagcggctactactggagctggatcaggcagccccccggcaagtgcctggagtggatcggcgagatcgaccacagcggcagcaccaactacaaccccagcctgaagagcagggtgaccatcagcgtggacaccagcaagaaccagttcagcctgaagctgagcagcgtgaccgccgccgacaccgccgtgtactactgcgccaggcagggcatccacggcctgaggtacttcgacctgtggggcaggggcaccctggtgaccgtgagcagcggcggcggcggcagcggcggcggcggcagcggcggcggcggcagcggcggcggcggcagcgagatcgtgatgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcaggaccacaacttcccctacaccttcggctgcggcaccaaggtggagatcaag

SEQ ID NO: 497 - DNA, 744 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagcagtacgtgacccccatcaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgagaagtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagc

SEQ ID NO: 498 - DNA, 744 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgagaagtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagcagtacgtgacccccatcaccttcggctgcggcaccaaggtggagatcaag

SEQ ID NO: 499 - DNA, 744 bp, Artificial Sequence
  Description of Artificial Sequence Synthetic polynucleotide
gacatccagatgacccagagccccagcagcgtgagcgccagcgtgggcgacagggtgaccatcacctgcagggccagccagggcatcagcagctggctggcctggtaccagcagaagcccggcaaggcccccaagctgctgatctacgccgccagcaacctgcagagcggcgtgcccagcaggttcagcggcagcggcagcggcaccgacttcaccctgaccatcagcagcctgcagcccgaggacttcgccacctactactgccagcagaagctgagcctgcccctgaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgagaagtactacgtggacagcgtgaagggcaggttcaccatcagcagggacaacgccaagaacagcctgtacctgcagatgaacagcctgagggccgaggacaccgccgtgtactactgcgccagggagggcggcgacagctggtaccacgccttcgacatctggggccagggcaccatggtgaccgtgagcagc

SEQ ID NO: 500 - DNA, 744 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgagaagtactacgtggacagcgtgaagggcaggttcaccatcagcagggacaacgccaagaacagcctgtacctgcagatgaacagcctgagggccgaggacaccgccgtgtactactgcgccagggagggcggcgacagctggtaccacgccttcgacatctggggccagggcaccatggtgaccgtgagcagcggcggcggcggcagcggcggcggcggcagcggcggcggcggcagcggcggcggcggcagcgacatccagatgacccagagccccagcagcgtgagcgccagcgtgggcgacagggtgaccatcacctgcagggccagccagggcatcagcagctggctggcctggtaccagcagaagcccggcaaggcccccaagctgctgatctacgccgccagcaacctgcagagcggcgtgcccagcaggttcagcggcagcggcagcggcaccgacttcaccctgaccatcagcagcctgcagcccgaggacttcgccacctactactgccagcagaagctgagcctgcccctgaccttcggctgcggcaccaaggtggagatcaag

SEQ ID NO: 501 - DNA, 750 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacggcgccagcagcctgcagagcggcgtgcccagcaggttcagcggcagcggcagcggcaccgacttcaccctgaccatcagcagcctgcagcccgaggacttcgccacctactactgccagcaggtgtacagcgcccccttcaccttcggctgcggcaccaaggtggagatcaagggcggcggcggcagcggcggcggcggcagcggcggcggcggcagcggcggcggcggcagccaggtgcagctgcaggagagcggccccggcctggtgaagcccagccagaccctgagcctgacctgcaccgtgagcggcggcagcatcagcagcggcggctactactggagctggatcaggcagcaccccggcaagtgcctggagtggatcggcagcatctactacagcggcagcacctactacaaccccagcctgaagagcagggtgaccatcagcgtggacaccagcaagaaccagttcagcctgaagctgagcagcgtgaccgccgccgacaccgccgtgtactactgcgccagggacaggctggactacagctacaactacggcatggacgtgtggggccagggcaccaccgtgaccgtgagcagc

SEQ ID NO: 502 - DNA, 750 bp, Artificial Sequence
  Description of Artificial Sequence Synthetic polynucleotide
caggtgcagctgcaggagagcggccccggcctggtgaagcccagccagaccctgagcctgacctgcaccgtgagcggcggcagcatcagcagcggcggctactactggagctggatcaggcagcaccccggcaagtgcctggagtggatcggcagcatctactacagcggcagcacctactacaaccccagcctgaagagcagggtgaccatcagcgtggacaccagcaagaaccagttcagcctgaagctgagcagcgtgaccgccgccgacaccgccgtgtactactgcgccagggacaggctggactacagctacaactacggcatggacgtgtggggccagggcaccaccgtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacggcgccagcagcctgcagagcggcgtgcccagcaggttcagcggcagcggcagcggcaccgacttcaccctgaccatcagcagcctgcagcccgaggacttcgccacctactactgccagcaggtgtacagcgcccccttcaccttcggctgcggcaccaaggtggagatcaag

SEQ ID NO: 503 - DNA, 744 bp, Artificial Sequence
  Description of Artificial Sequence Synthetic polynucleotide
gagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcaggtggacaactacccccccaccttcggctgcggcaccaaggtggagatcaagggcggcggcggcagcggcggcggcggcagcggcggcggcggcagcggcggcggcggcagccaggtgcagctgcaggagagcggccccggcctggtgaagcccagcgagaccctgagcctgacctgcgccgtgagcggctacagcatcagcagcggctactactggggctggatcaggcagccccccggcaagtgcctggagtggatcggcagcatctaccacagcggcagcaccaactacaaccccagcctgaagagcagggtgaccatcagcgtggacaccagcaagaaccagttcagcctgaagctgagcagcgtgaccgccgccgacaccgccgtgtactactgcgccaggctgcccccctggttcggcttcagctacttcgacctgtggggcaggggcaccctggtgaccgtgagcagc

SEQ ID NO: 504 - DNA, 744 bp, Artificial Sequence
  Description of Artificial Sequence Synthetic polynucleotide
caggtgcagctgcaggagagcggccccggcctggtgaagcccagcgagaccctgagcctgacctgcgccgtgagcggctacagcatcagcagcggctactactggggctggatcaggcagccccccggcaagtgcctggagtggatcggcagcatctaccacagcggcagcaccaactacaaccccagcctgaagagcagggtgaccatcagcgtggacaccagcaagaaccagttcagcctgaagctgagcagcgtgaccgccgccgacaccgccgtgtactactgcgccaggctgcccccctggttcggcttcagctacttcgacctgtggggcaggggcaccctggtgaccgtgagcagcggcggcggcggcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcaggtggacaactacccccccaccttcggctgcggcaccaaggtggagatcaag

SEQ ID NO: 505 - DNA, 750 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagc

SEQ ID NO: 506 - DNA, 750 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaagcaggacggcagcgagaagtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaag

SEQ ID NO: 507 - DNA, 741 bp, Artificial Sequence
  Description of Artificial Sequence Synthetic polynucleotide
gagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagtacgacaacctgcccaccttcggctgcggcaccaaggtggagatcaagggcggcggcggcagcggcggcggcggcagcggcggcggcggcagcggcggcggcggcagccagctgcagctgcaggagagcggccccggcctggtgaagcccagcgagaccctgagcctgacctgcaccgtgagcggcggcagcatcagcagcagcagctactactggggctggatcaggcagccccccggcaagtgcctggagtggatcggcagcatctactacagcggcagcacctactacaaccccagcctgaagagcagggtgaccatcagcgtggacaccagcaagaaccagttcagcctgaagctgagcagcgtgaccgccgccgacaccgccgtgtactactgcgccagggagaccgcccacgacgtgcacggcatggacgtgtggggccagggcaccaccgtgaccgtgagcagc

SEQ ID NO: 508 - DNA, 741 bp, Artificial Sequence
  Description of Artificial Sequence Synthetic polynucleotide
cagctgcagctgcaggagagcggccccggcctggtgaagcccagcgagaccctgagcctgacctgcaccgtgagcggcggcagcatcagcagcagcagctactactggggctggatcaggcagccccccggcaagtgcctggagtggatcggcagcatctactacagcggcagcacctactacaaccccagcctgaagagcagggtgaccatcagcgtggacaccagcaagaaccagttcagcctgaagctgagcagcgtgaccgccgccgacaccgccgtgtactactgcgccagggagaccgcccacgacgtgcacggcatggacgtgtggggccagggcaccaccgtgaccgtgagcagcggcggcggcggcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagtacgacaacctgcccaccttcggctgcggcaccaaggtggagatcaag

SEQ ID NO: 509 - DNA, 750 bp, Artificial Sequence
  Description of Artificial Sequence Synthetic polynucleotide
gagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaagagggccaccggcatccccgccaggttcagcggcagcggcagcggcaccgacttcaccctgaccatcagcagcctggagcccgaggacttcgccgtgtactactgccagcagagcagcaaccaccccagcaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcagcagcgtgaaggtgagctgcaaggccagcggcggcaccttcagcagctacgccatcagctgggtgaggcaggcccccggccagtgcctggagtggatgggcagcatcatccccatcttcggcaccgccaactacgcccagaagttccagggcagggtgaccatcaccgccgacgagagcaccagcaccgcctacatggagctgagcagcctgaggagcgaggacaccgccgtgtactactgcgccagggaggtgggctacggctggtacaccaagatcgccttcgacatctggggccagggcaccatggtgaccgtgagcagc

SEQ ID NO: 510 - DNA, 750 bp, Artificial Sequence
  Description of Artificial Sequence Synthetic polynucleotide
caggtgcagctggtgcagagcggcgccgaggtgaagaagcccggcagcagcgtgaaggtgagctgcaaggccagcggcggcaccttcagcagctacgccatcagctgggtgaggcaggcccccggccagtgcctggagtggatgggcagcatcatccccatcttcggcaccgccaactacgcccagaagttccagggcagggtgaccatcaccgccgacgagagcaccagcaccgcctacatggagctgagcagcctgaggagcgaggacaccgccgtgtactactgcgccagggaggtgggctacggctggtacaccaagatcgccttcgacatctggggccagggcaccatggtgaccgtgagcagcggcggcggcggcagcggcggcggcggcagcggcggcggcggcagcggcggcggcggcagcgagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaagagggccaccggcatccccgccaggttcagcggcagcggcagcggcaccgacttcaccctgaccatcagcagcctggagcccgaggacttcgccgtgtactactgccagcagagcagcaaccaccccagcaccttcggctgcggcaccaaggtggagatcaag

SEQ ID NO: 511 - DNA, 765 bp, Artificial Sequence
  Description of Artificial Sequence Synthetic polynucleotide
gacatcgtgatgacccagagccccctgagcctgcccgtgacccccggcgagcccgccagcatcagctgcaggagcagccagagcctgctgcacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgggcgtgcccctgaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccagctactacatgcactgggtgaggcaggcccccggccagtgcctggagtggatgggcatcatcaaccccagcggcggcagcaccacctacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagc

SEQ ID NO: 512 - DNA, 765 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcaccagctactacatgcactgggtgaggcaggcccccggccagtgcctggagtggatgggcatcatcaaccccagcggcggcagcaccacctacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagcggcggcggcggcagcggcggcggcggcagcggcggcggcggcagcggcggcggcggcagcgacatcgtgatgacccagagccccctgagcctgcccgtgacccccggcgagcccgccagcatcagctgcaggagcagccagagcctgctgcacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggccctgggcgtgcccctgaccttcggctgcggcaccaaggtggagatcaag

SEQ ID NO: 513 - DNA, 765 bp, Artificial Sequence
  Description of Artificial Sequence Synthetic polynucleotide
gacatcgtgatgacccagagccccctgagcctgcccgtgacccccggcgagcccgccagcatcagctgcaggagcagccagagcctgctgtacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggacgtggccctgcccatcaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcagcggctactacatgcactgggtgaggcaggcccccggccagtgcctggagtggatgggcatgatcaacccctacggcggcagcaccaggtacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagc

SEQ ID NO: 514 - DNA, 765 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcagcggctactacatgcactgggtgaggcaggcccccggccagtgcctggagtggatgggcatgatcaacccctacggcggcagcaccaggtacgcccagaagttccagggcagggtgaccatgaccagggacaccagcaccagcaccgtgtacatggagctgagcagcctgaggagcgaggacaccgccgtgtactactgcgccagggaggccgccgacggcttcgtgggcgagaggtacttcgacctgtggggcaggggcaccctggtgaccgtgagcagcggcggcggcggcagcggcggcggcggcagcggcggcggcggcagcggcggcggcggcagcgacatcgtgatgacccagagccccctgagcctgcccgtgacccccggcgagcccgccagcatcagctgcaggagcagccagagcctgctgtacagcaacggctacaactacctggactggtacctgcagaagcccggccagagcccccagctgctgatctacctgggcagcaacagggccagcggcgtgcccgacaggttcagcggcagcggcagcggcaccgacttcaccctgaagatcagcagggtggaggccgaggacgtgggcgtgtactactgcatgcaggacgtggccctgcccatcaccttcggctgcggcaccaaggtggagatcaag

SEQ ID NO: 515 - DNA, 741 bp, Artificial Sequence
  Description of Artificial Sequence Synthetic polynucleotide
gacatccagatgacccagagccccagcagcgtgagcgccagcgtgggcgacagggtgaccatcacctgcagggccagccagggcatcgacagctggctggcctggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggcccacagctaccccctgaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcgagatctactacatgcactgggtgaggcaggcccccggccagtgcctggagtggatgggcatcatcaaccccagcagcggcagcaccgtgtacgcccagaagttccagggcagggtgaccatgaccagggacaccagcaccagcaccgtgtacatggagctgagcagcctgaggagcgaggacaccgccgtgtactactgcgccaggggcgccggctacgacgacgaggacatggacgtgtggggcaagggcaccaccgtgaccgtgagcagc

SEQ ID NO: 516 - DNA, 741 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcgagatctactacatgcactgggtgaggcaggcccccggccagtgcctggagtggatgggcatcatcaaccccagcagcggcagcaccgtgtacgcccagaagttccagggcagggtgaccatgaccagggacaccagcaccagcaccgtgtacatggagctgagcagcctgaggagcgaggacaccgccgtgtactactgcgccaggggcgccggctacgacgacgaggacatggacgtgtggggcaagggcaccaccgtgaccgtgagcagcggcggcggcggcagcggcggcggcggcagcggcggcggcggcagcggcggcggcggcagcgacatccagatgacccagagccccagcagcgtgagcgccagcgtgggcgacagggtgaccatcacctgcagggccagccagggcatcgacagctggctggcctggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcaggcccacagctaccccctgaccttcggctgcggcaccaaggtggagatcaag

SEQ ID NO: 517 - DNA, 744 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatctacaactacctgaactggtaccagcagaagcccggcaaggcccccaagctgctgatctacgccgccagcaacctgcacagcggcgtgcccagcaggttcagcggcagcggcagcggcaccgacttcaccctgaccatcagcagcctgcagcccgaggacttcgccacctactactgccagcaggccttccacgtgcccatcaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcggcggctactggatgagctgggtgaggcaggcccccggcaagtgcctggagtgggtggccaacatcaaccaggacggcagcgaggagtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagc

SEQ ID NO: 518 - DNA, 744 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcggcggctactggatgagctgggtgaggcaggcccccggcaagtgcctggagtgggtggccaacatcaaccaggacggcagcgaggagtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatctacaactacctgaactggtaccagcagaagcccggcaaggcccccaagctgctgatctacgccgccagcaacctgcacagcggcgtgcccagcaggttcagcggcagcggcagcggcaccgacttcaccctgaccatcagcagcctgcagcccgaggacttcgccacctactactgccagcaggccttccacgtgcccatcaccttcggctgcggcaccaaggtggagatcaag

SEQ ID NO: 519 - DNA, 744 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatctacaactacctgaactggtaccagcagaagcccggcaaggcccccaagctgctgatctacgccgccagcagcacccagagcggcgtgcccagcaggttcagcggcagcggcagcggcaccgacttcaccctgaccatcagcagcctgcagcccgaggacttcgccacctactactgccagcaggccttccacgtgcccatcaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttccccggctactggatgagctgggtgaggcaggcccccggcaagtgcctggagtgggtggccaacatcaaccaggacggcagcgaggtgtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagc

SEQ ID NO: 520 - DNA, 744 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttccccggctactggatgagctgggtgaggcaggcccccggcaagtgcctggagtgggtggccaacatcaaccaggacggcagcgaggtgtactacgtggacagcgtgaagggcaggttcaccatcagcagggacaacgccaagaacagcctgtacctgcagatgaacagcctgagggccgaggacaccgccgtgtactactgcgccagggaggccaactactacggcaacgtgggcgacgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatctacaactacctgaactggtaccagcagaagcccggcaaggcccccaagctgctgatctacgccgccagcagcacccagagcggcgtgcccagcaggttcagcggcagcggcagcggcaccgacttcaccctgaccatcagcagcctgcagcccgaggacttcgccacctactactgccagcaggccttccacgtgcccatcaccttcggctgcggcaccaaggtggagatcaag

SEQ ID NO: 521 - DNA, 750 bp, Artificial Sequence
  Description of Artificial Sequence Synthetic polynucleotide
gacatccagatgacccagagccccagcagcctgagcgccagcgtgggcgacagggtgaccatcacctgcagggccagccagagcatctactactacctgaactggtaccagcagaagcccggcaaggcccccaagctgctgatctacgccgccagcagcaggcagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaagggcggcggcggcagcggcggcggcggcagcggcggcggcggcagcggcggcggcggcagcgaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgaggtgtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagc

SEQ ID NO: 522 - DNA, 750 bp, Artificial Sequence
  Description of Artificial Sequence Synthetic polynucleotide
gaggtgcagctggtggagagcggcggcggcctggtgcagcccggcggcagcctgaggctgagctgcgccgccagcggcttcaccttcagcagctactggatgagctgggtgaggcaggcccccggcaagtgcctggagtgggtggccaacatcaaccaggacggcagcgaggtgtactacgtggacagcgtgaagggcaggttcaccatcagcagggacaacgccaagaacagcctgtacctgcagatgaacagcctgagggccgaggacaccgccgtgtactactgcgccagggacgtgggccccggcatcgcctaccagggccacttcgactactggggccagggcaccctggtgaccgtgagcagcggcggcggcggcagcggcggcggcggcagcggcggcggcggcagcggcggcggcggcagcgacatccagatgacccagagccccagcagcctgagcgccagcgtgggcgacagggtgaccatcacctgcagggccagccagagcatctactactacctgaactggtaccagcagaagcccggcaaggcccccaagctgctgatctacgccgccagcagcaggcagagcggcgtgcccagcaggttcagcggcagcggcagcggcaccgacttcaccctgaccatcagcagcctgcagcccgaggacttcgccacctactactgccagcaggtgtacgacacccccctgaccttcggctgcggcaccaaggtggagatcaag

SEQ ID NO: 523 - DNA, 744 bp, Artificial Sequence
  Description of Artificial Sequence Synthetic polynucleotide
gacatccagatgacccagagccccagcagcgtgagcgccagcgtgggcgacagggtgaccatcacctgcgaggccagcaagggcatcagcagctggctggcctggtaccagcagaagcccggcaaggcccccaagctgctgatctacgccgccagcgacctgcagagcggcgtgcccagcaggttcagcggcagcggcagcggcaccgacttcaccctgaccatcagcagcctgcagcccgaggacttcgccacctactactgccagcaggccttcctgttcccccccaccttcggctgcggcaccaaggtggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcagcaactactacatgcactgggtgaggcaggcccccggccagtgcctggagtggatgggctggatcaaccccttcagcggcggcaccaggtacgcccagaagttccagggcagggtgaccatgaccagggacaccagcaccagcaccgtgtacatggagctgagcagcctgaggagcgaggacaccgccgtgtactactgcgccagggacgtgggcagcagcgcctactactacatggacgtgtggggcaagggcaccaccgtgaccgtgagcagc

SEQ ID NO: 524 - DNA, 744 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacaccttcagcaactactacatgcactgggtgaggcaggcccccggccagtgcctggagtggatgggctggatcaaccccttcagcggcggcaccaggtacgcccagaagttccagggcagggtgaccatgaccagggacaccagcaccagcaccgtgtacatggagctgagcagcctgaggagcgaggacaccgccgtgtactactgcgccagggacgtgggcagcagcgcctactactacatggacgtgtggggcaagggcaccaccgtgaccgtgagcagcggcggcggcggcagcggcggcggcggcagcggcggcggcggcagcggcggcggcggcagcgacatccagatgacccagagccccagcagcgtgagcgccagcgtgggcgacagggtgaccatcacctgcgaggccagcaagggcatcagcagctggctggcctggtaccagcagaagcccggcaaggcccccaagctgctgatctacgccgccagcgacctgcagagcggcgtgcccagcaggttcagcggcagcggcagcggcaccgacttcaccctgaccatcagcagcctgcagcccgaggacttcgccacctactactgccagcaggccttcctgttcccccccaccttcggctgcggcaccaaggtggagatcaag

SEQ ID NO: 525 - DNA, 750 bp, Artificial Sequence
  Description of Artificial Sequence Synthetic polynucleotide
gagatcgtgctgacccagagccccggcaccctgagcctgagccccggcgagagggccaccctgagctgcagggccagccagagcgtgagcagcagcttcctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagctggacagccccccccccaccttcggctgcggcaccaaggtggagatcaagggcggcggcggcagcggcggcggcggcagcggcggcggcggcagcggcggcggcggcagcgaggtgcagctggtgcagagcggcgccgaggtgaagaagcccggcgagagcctgaagatcagctgcaagggcagcggctacagcttcaccagctactggatcggctgggtgaggcagatgcccggcaagtgcctggagtggatgggcagcatctaccccggcgacagcgacaccaggtacagccccagcttccagggccaggtgaccatcagcgccgacaagagcatcagcaccgcctacctgcagtggagcagcctgaaggccagcgacaccgccatgtactactgcgccagggagctggcctacggcgactacaagggcggcgtggactactggggccagggcaccctggtgaccgtgagcagc

SEQ ID NO: 526 - DNA, 750 bp, Artificial Sequence
  Description of Artificial Sequence Synthetic polynucleotide
gaggtgcagctggtgcagagcggcgccgaggtgaagaagcccggcgagagcctgaagatcagctgcaagggcagcggctacagcttcaccagctactggatcggctgggtgaggcagatgcccggcaagtgcctggagtggatgggcagcatctaccccggcgacagcgacaccaggtacagccccagcttccagggccaggtgaccatcagcgccgacaagagcatcagcaccgcctacctgcagtggagcagcctgaaggccagcgacaccgccatgtactactgcgccagggagctggcctacggcgactacaagggcggcgtggactactggggccagggcaccctggtgaccgtgagcagcggcggcggcggcagcggcggcggcggcagcggcggcggcggcagcggcggcggcggcagcgagatcgtgctgacccagagccccggcaccctgagcctgagccccggcgagagggccaccctgagctgcagggccagccagagcgtgagcagcagcttcctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagctggacagccccccccccaccttcggctgcggcaccaaggtggagatcaag

SEQ ID NO: 527 - protein, 13 aa, Artificial Sequence
  Description of Artificial Sequence Synthetic peptide
  VARIANT (4)..(4): Ala, Val, Leu, Ile, Pro, Phe, Trp, Gly, Ser, Thr, Cys, Asn, Gln, or Tyr
GAPXGAAAGWFDP

SEQ ID NO: 528 - protein, 9 aa, Artificial Sequence
  Description of Artificial Sequence Synthetic peptide
FTFSSYAMS

SEQ ID NO: 529 - protein, 20 aa, Artificial Sequence
  Description of Artificial Sequence Synthetic peptide
ARPRAYYDSSGFKVNYGMDV

SEQ ID NO: 530 - protein, 9 aa, Artificial Sequence
  Description of Artificial Sequence Synthetic peptide
FTFSSYAMS

SEQ ID NO: 531 - protein, 16 aa, Artificial Sequence
  Description of Artificial Sequence Synthetic peptide
AREGHSSSYYDHAFDI

SEQ ID NO: 532 - protein, 9 aa, Artificial Sequence
  Description of Artificial Sequence Synthetic peptide
GSISSYYWS

SEQ ID NO: 533 - protein, 17 aa, Artificial Sequence
  Description of Artificial Sequence Synthetic peptide
ARVGGVYSTIETYGMDV

SEQ ID NO: 534 - protein, 9 aa, Artificial Sequence
  Description of Artificial Sequence Synthetic peptide
GSFSGYYWS

SEQ ID NO: 535 - protein, 13 aa, Artificial Sequence
  Description of Artificial Sequence Synthetic peptide
ARQGIHGLRYFDL

SEQ ID NO: 536 - protein, 9 aa, Artificial Sequence
  Description of Artificial Sequence Synthetic peptide
FTFSSYWMS

SEQ ID NO: 537 - protein, 14 aa, Artificial Sequence
  Description of Artificial Sequence Synthetic peptide
AREANYYGNVGDDY

SEQ ID NO: 538 - protein, 9 aa, Artificial Sequence
  Description of Artificial Sequence Synthetic peptide
FTFSSYWMS

SEQ ID NO: 539 - protein, 14 aa, Artificial Sequence
  Description of Artificial Sequence Synthetic peptide
AREGGDSWYHAFDI

SEQ ID NO: 540 - protein, 11 aa, Artificial Sequence
  Description of Artificial Sequence Synthetic peptide
GSISSGGYYWS

SEQ ID NO: 541 - protein, 15 aa, Artificial Sequence
  Description of Artificial Sequence Synthetic peptide
ARDRLDYSYNYGMDV

SEQ ID NO: 542 - protein, 10 aa, Artificial Sequence
  Description of Artificial Sequence Synthetic peptide
YSISSGYYWG

SEQ ID NO: 543 - protein, 14 aa, Artificial Sequence
  Description of Artificial Sequence Synthetic peptide
ARLPPWFGFSYFDL

SEQ ID NO: 544 - protein, 9 aa, Artificial Sequence
  Description of Artificial Sequence Synthetic peptide
FTFSSYWMS

SEQ ID NO: 545 - protein, 16 aa, Artificial Sequence
  Description of Artificial Sequence Synthetic peptide
ARDVGPGIAYQGHFDY

SEQ ID NO: 546 - protein, 11 aa, Artificial Sequence
  Description of Artificial Sequence Synthetic peptide
GSISSSSYYWG

SEQ ID NO: 547 - protein, 13 aa, Artificial Sequence
  Description of Artificial Sequence Synthetic peptide
ARETAHDVHGMDV

SEQ ID NO: 548 - protein, 9 aa, Artificial Sequence
  Description of Artificial Sequence Synthetic peptide
GTFSSYAIS

SEQ ID NO: 549 - protein, 16 aa, Artificial Sequence
  Description of Artificial Sequence Synthetic peptide
AREVGYGWYTKIAFDI

SEQ ID NO: 550 - protein, 9 aa, Artificial Sequence
  Description of Artificial Sequence Synthetic peptide
YTFTSYYMH

SEQ ID NO: 551 - protein, 16 aa, Artificial Sequence
  Description of Artificial Sequence Synthetic peptide
AREAADGFVGERYFDL

SEQ ID NO: 552 - protein, 9 aa, Artificial Sequence
  Description of Artificial Sequence Synthetic peptide
YTFSGYYMH

SEQ ID NO: 553 - protein, 16 aa, Artificial Sequence
  Description of Artificial Sequence Synthetic peptide
AREAADGFVGERYFDL

SEQ ID NO: 554 - protein, 9 aa, Artificial Sequence
  Description of Artificial Sequence Synthetic peptide
YTFEIYYMH

SEQ ID NO: 555 - protein, 13 aa, Artificial Sequence
  Description of Artificial Sequence Synthetic peptide
ARGAGYDDEDMDV

SEQ ID NO: 556 - protein, 9 aa, Artificial Sequence
  Description of Artificial Sequence Synthetic peptide
FTFGGYWMS

SEQ ID NO: 557 - protein, 14 aa, Artificial Sequence
  Description of Artificial Sequence Synthetic peptide
AREANYYGNVGDDY

SEQ ID NO: 558 - protein, 9 aa, Artificial Sequence
  Description of Artificial Sequence Synthetic peptide
FTFPGYWMS

SEQ ID NO: 559 - protein, 14 aa, Artificial Sequence
  Description of Artificial Sequence Synthetic peptide
AREANYYGNVGDDY

SEQ ID NO: 560 - protein, 9 aa, Artificial Sequence
  Description of Artificial Sequence Synthetic peptide
FTFSSYWMS

SEQ ID NO: 561 - protein, 16 aa, Artificial Sequence
  Description of Artificial Sequence Synthetic peptide
ARDVGPGIAYQGHFDY

SEQ ID NO: 562 - protein, 9 aa, Artificial Sequence
  Description of Artificial Sequence Synthetic peptide
YTFSNYYMH

SEQ ID NO: 563 - protein, 14 aa, Artificial Sequence
  Description of Artificial Sequence Synthetic peptide
ARDVGSSAYYYMDV

SEQ ID NO: 564 - protein, 9 aa, Artificial Sequence
  Description of Artificial Sequence Synthetic peptide
YSFTSYWIG

SEQ ID NO: 565 - protein, 15 aa, Artificial Sequence
  Description of Artificial Sequence Synthetic peptide
ARELAYGDYKGGVDY

SEQ ID NO: 566 - protein, 5 aa, Artificial Sequence
  Description of Artificial Sequence Synthetic peptide
SYYMH

SEQ ID NO: 567 - protein, 17 aa, Artificial Sequence
  Description of Artificial Sequence Synthetic peptide
GAPNYGDTTHDYYYMDV

SEQ ID NO: 568 - protein, 5 aa, Artificial Sequence
  Description of Artificial Sequence Synthetic peptide
GYYMH

SEQ ID NO: 569 - protein, 15 aa, Artificial Sequence
  Description of Artificial Sequence Synthetic peptide
DTGEYYDTDDHGMDV

SEQ ID NO: 570 - protein, 5 aa, Artificial Sequence
  Description of Artificial Sequence Synthetic peptide
SYAMS

SEQ ID NO: 571 - protein, 12 aa, Artificial Sequence
  Description of Artificial Sequence Synthetic peptide
DGGYYDSGAGDY

SEQ ID NO: 572 - protein, 5 aa, Artificial Sequence
  Description of Artificial Sequence Synthetic peptide
SYYMH

SEQ ID NO: 573 - protein, 16 aa, Artificial Sequence
  Description of Artificial Sequence Synthetic peptide
EGAGFAYGMDYYYMDV

SEQ ID NO: 574 - protein, 5 aa, Artificial Sequence
  Description of Artificial Sequence Synthetic peptide
GYYWS

SEQ ID NO: 575 - protein, 9 aa, Artificial Sequence
  Description of Artificial Sequence Synthetic peptide
ARGPWSFDP

SEQ ID NO: 576 - protein, 5 aa, Artificial Sequence
  Description of Artificial Sequence Synthetic peptide
SYAIS

SEQ ID NO: 577 - protein, 16 aa, Artificial Sequence
  Description of Artificial Sequence Synthetic peptide
GDSSIRHAYYYYGMDV

SEQ ID NO: 578 - protein, 7 aa, Artificial Sequence
  Description of Artificial Sequence Synthetic peptide
SSSYYWG

SEQ ID NO: 579 - protein, 11 aa, Artificial Sequence
  Description of Artificial Sequence Synthetic peptide
GSDRFHPYFDY

SEQ ID NO: 580 - protein, 113 aa, Artificial Sequence [sequence omitted]

SEQ ID NO: 581 - protein, 7 aa, Artificial Sequence
  Description of Artificial Sequence Synthetic peptide
DASNRAT

SEQ ID NO: 582 - protein, 9 aa, Artificial Sequence
  Description of Artificial Sequence Synthetic peptide
QQFDTWPPT

SEQ ID NO: 583 - protein, 117 aa, Artificial Sequence; identical to SEQ ID NO: 124
  Description of Artificial Sequence Synthetic polypeptide

SEQ ID NO: 584 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 585 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 586 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 587 - protein, 15 aa, Artificial Sequence
  Description of Artificial Sequence Synthetic peptide
ARGAPQGAAAGWFDP

SEQ ID NO: 588 - protein, 13 aa, Artificial Sequence
  Description of Artificial Sequence Synthetic peptide
GAPQGAAAGWFDP

SEQ ID NO: 589 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 590 - protein, 15 aa, Artificial Sequence
  Description of Artificial Sequence Synthetic peptide
ARGAPLGAAAGWFDP

SEQ ID NO: 591 - protein, 13 aa, Artificial Sequence
  Description of Artificial Sequence Synthetic peptide
GAPLGAAAGWFDP

SEQ ID NO: 592 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 593 - protein, 15 aa, Artificial Sequence
  Description of Artificial Sequence Synthetic peptide
ARGAPFGAAAGWFDP

SEQ ID NO: 594 - protein, 13 aa, Artificial Sequence
  Description of Artificial Sequence Synthetic peptide
GAPFGAAAGWFDP

SEQ ID NO: 595 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 596 - protein, 15 aa, Artificial Sequence
  Description of Artificial Sequence Synthetic peptide
ARGAPVGAAAGWFDP

SEQ ID NO: 597 - protein, 13 aa, Artificial Sequence
  Description of Artificial Sequence Synthetic peptide
GAPVGAAAGWFDP

SEQ ID NO: 598 - protein, 347 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 599 - protein, 343 aa, Macaca fascicularis
DPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGKQGVVQGAIGGAGVTVLLALCLCLIFFTVKTHRRKAARTAVGRIDTHPATGPTSSKHQKKSKLHGATETSGCSGTTLTVEMDEELHYASLNFHGMNPSEDTSTEYSEVRTQ